(19) United States
(12) Patent Application Publication
(10) Pub. No.: US 2026/0176377 A1
(43) Pub. Date: Jun. 25, 2026

(54) AGONISTIC SPLIT ANTIBODIES

(71) Applicant: Hoffmann-La Roche Inc., Little Falls, NJ (US)

(72) Inventors: Petra Elisabeth Marilena BALBI, Schlieren (CH); Jack Anthony BATES, Oxford (GB); Samuele CALABRO, Schlieren (CH); Laura CODARRI DEAK, Au (CH); Stephan GASSER, Fislisbach (CH); Guy GEORGES, Habach (DE); Xavier GUERIPEL, Zurich (CH); Ralf Joerg HOSSE, Mettmenstetten (CH); Christian KLEIN, Weilheim (DE); Stéphane Gérard Alain LECLAIR, Gauting (DE); Ekkehard MOESSNER, Kreuzlingen (CH); Christian MUELLER, Rheinfelden (DE); Diana Angela PIPPIG, Muenchen (DE); Laurene POUSSE, Schlieren (CH); Camilla Elizabeth TREVOR, Bergdietikon (CH); Pablo UMAÑA FERNÁNDEZ, Lachen (CH)

(73) Assignee: Hoffmann-La Roche Inc., Little Falls, NJ (US)

(21) Appl. No.: 19/348,510

(22) Filed: Oct. 2, 2025

Related U.S. Application Data
(63) Continuation of application No. PCT/EP2024/058832, filed on Apr. 2, 2024.

(30) Foreign Application Priority Data
Apr. 3, 2023 (EP) 23166352.7

(51) Int. Cl. C07K 16/40; C07K 16/28; C07K 16/32

(52) U.S. Cl. CPC C07K 16/40; C07K 16/2803; C07K 16/2818; C07K 16/2863; C07K 16/2866; C07K 16/32; C07K 2317/31; C07K 2317/522; C07K 2317/569

(57) ABSTRACT

The application relates to pairs of antigen binding molecules, each molecule comprising a target-binding domain, a cytokine receptor-binding domain and an Fc domain, wherein the target-binding domains simultaneously bind the target antigen and the cytokine receptor-binding domains bind subunits of a cytokine receptor complex. Biparatopic assembly of the cytokine receptor-binding domains in presence of the target antigen allows to selectively activate a cytokine receptor and effectively mimic cytokine activity in a targeted manner.

### CROSS-REFERENCE TO RELATED APPLICATIONS

This application is a continuation of International Application No. PCT/EP2024/058832 filed Apr. 2, 2024, which claims the benefit of and priority to European Patent Application No. 23166352.7, filed Apr. 3, 2023, each of which is incorporated herein by reference in its entirety.

### REFERENCE TO AN ELECTRONIC SEQUENCE LISTING

The instant application contains a Sequence Listing submitted electronically in XML format and is hereby incorporated herein by reference in its entirety. Said XML copy, created on Sep. 15, 2025, is named P38387-US-1_SEQ_LISTING.xml, and is 349,009 bytes in size.

### FIELD OF THE INVENTION

The application relates to pairs of antigen binding molecules, each molecule comprising a target-binding domain, a cytokine receptor-binding domain and an Fc domain, wherein the target-binding domains simultaneously bind the target antigen and the cytokine receptor-binding domains bind subunits of a cytokine receptor complex. Biparatopic assembly of the cytokine receptor-binding domains in presence of the target antigen allows to selectively activate a cytokine receptor and effectively mimic cytokine activity in a targeted manner.

### BACKGROUND OF THE INVENTION

In recent years, immunotherapy treatments for cancer have grown dramatically, and cancer immunotherapy is becoming a major strategy for combating disease. For many cancer types, immune checkpoint modulators, including anti-PD-1, have become the standard of care. However, despite all the advances made in the field of cancer immunotherapy treatment in recent years, a significant proportion of patients still fail to respond to available immunotherapies because of intrinsic or adaptive mechanisms of resistance. Cancer immunotherapy patients with non-inflamed immune conditions are more likely to not respond to immunotherapies. Immune cell infiltration in tumors has been shown to correlate with the ability of patients to respond to immunotherapy treatments. Developing new therapies aimed at increasing immune cell infiltration and enhancing immunogenicity is essential for patients.

In parallel with the above developments, cytokines have gained great interest as potential cancer treatments. IFNγ (interferon gamma or IFN-γ) is a cytokine that is mainly produced by activated lymphocytes, such as CD4⁺ and CD8⁺ T cells, and natural killer cells (NK cells) in response to immune or inflammatory stimuli. IFNγ is a homodimer and its receptors (IFNγR1 and IFNγR2) are expressed across hematopoietic and non-hematopoietic cells. On the cell surface, IFNγR1 is stably expressed whereas IFNγR2 is differentially expressed and regulates IFNγ. When IFNγ binds to its receptors, Janus kinases JAK1 and JAK2 are recruited and activated, which phosphorylate and activate STAT1. A phosphorylated STAT1 translocates to the nucleus, binds promoters, and modulates gene transcription under the control of IFNγ.

In contrast to other cancer therapies currently on the market or in development, IFNγ can act on both tumor cells and immune cells including T cells and dendritic cells. The effects of IFNγ on different cell types have several benefits, which include 1) enhanced expression of MHC-I molecules on the surface of both tumor cells and antigen-presenting cells, 2) the recruitment of immune cells to the tumor site through the induction of CXCL9, CXCL10 and CXCL11 production and 3) the ability to increase tumor antigen cross-presentation with a subsequent enhancement of an anti-tumor immune response. In addition to these effects, IFNγ promotes the formation of a Th1 environment, monocyte differentiation, macrophage polarization, and angiogenesis. As IFNγ receptors are expressed on a wide range of cell types, sink effects may diminish its activity.

IFNγ can also show undesirable side effects. In addition, problems with administration, bioavailabilty and short half-life may arise. Thus, there is a need for new molecules able to selectively activate the IFNγ pathway at the tumor site.

Targeting the Interleukin 2 (IL-2) pathway as a cancer immunotherapy strategy has a long and ramified history, serving as testament to both the triumphs and the complexities of this clinical approach. IL-2 is a cytokine that activates lymphocytes and natural killer (NK) cells. Efficacy in the clinic is often overshadowed by reports of IL-2-associated toxicity linked to peripheral T cell activity and CD25-mediated complications.

Cytokines are powerful immune modulators that initiate signaling through receptor dimerization, but natural cytokines have some limitations as therapeutics. Low stability and difficulties in the production process are just some of them. It is known for some time that antibodies can induce signaling on cells, and therefore substitute a natural ligand.

In the recent literature there is growing evidence that bringing together heterodimeric cytokine receptors using both antibody and non-antibody based protein scaffolds is a viable strategy to mimic the activity of native cytokines. For instance, Moraga and colleagues provide an early example of using diabodies as surrogate ligands for Erythropoietin receptor (EpoR) (Moraga et al. Cell 160, 1196-1208 (2015)). Researchers at Teneobio combined heavy chain-only antibodies (VHHs fused to an Fc domain) against different epitopes on the Interleukin-2 receptors (IL-2Rβ and IL-2Rγ) into a bispecific effectorless IgG₄ Fc (CH1 deleted) using knob-in-hole technology. While monospecific anti-IL-2Rβ or anti-IL-2Rγ alone or in mixture did not induce STAT5 phosphorylation on human CD8⁺ T cells, bispecific anti-IL-2Rβγ antibodies showed varying levels of agonist activity (Harris, K. E., et al. Sci Rep 11(1): 10592 (2021)). A similar approach as described by Teneobio, was undertaken by scientists at Synthekine, which described the functional induction of signaling of the two interleukin receptors (IL-2Rβ and IL-2Rγ) via single domain antibodies (sdAbs; WO 2022/032040 A1), and reviewed by Saxton and colleagues (Saxton, R. A., et al. Nat Rev Drug Discov. 22, 21-37 (2022)). This approach was further expanded by researchers at Stanford University where the authors presented a strategy to discovery cytokine surrogate agonists by using modular ligands like VHH or scFv human for interleukin-2/15, type-I interferon, and interleukin-10 receptors. Interestingly, they also identified functional, non-natural assemblies like the IL-2Rβ/IL-10Rβ heterodimer (Yen, M., et al. Cell 185(8): 1414-1430 e1419 (2022)). The same authors also discuss a structure-based approach for the decoupling of the pro- and anti-inflammatory functions of interleukin-10 (Saxton, R. A., et al. Science 371(6535) (2021)). Two academic research groups from Czech Republic and Israel reported together the discovery of non-antibody based scaffolds that mimic the cytokine IFNλ. Combinatorial libraries derived from several established small protein scaffolds were used to identify variants capable of binding to the IFNλR1 or IL-10Rβ and induce functional signaling (Kolarova, L., et al. FEBS J 289(9): 2672-2684 (2022)). Reducing the size of the agonistic modules was addressed by researchers at Medikine. They obtained molecules selected from peptide libraries by screens designed to identify molecules binding simultaneously to the Rα and γc subunits of the human IL-7 receptor. Those peptides, with an molecular weight of less than 5 kDa fused to an IgGn-Fc domain exhibits biological properties similar to those of IL-7 in vitro, and when administered to non-human primates (Dower, W., et al. Journal for Immuno Therapy of Cancer 8 (Suppl 3): A341-A342 (2020)).

Due to the pleiotropic effects of cytokines, there is a need for novel approaches to selectively activate cytokine receptors and effectively mimic cytokine activity.

### SUMMARY OF THE INVENTION

The present invention relates to novel antigen binding molecules comprising cytokine receptor-binding domains, which, under the desired conditions of biparatopic assembly on a target antigen, serve as cytokine mimetics selectively activating the receptor pathway.

The present invention relates to a pair of antigen binding molecules that specifically bind to a target antigen, comprising a) a first antigen binding molecule comprising i) a first target-binding domain, ii) a first cytokine receptor-binding domain, and iii) a Fc domain; and b) a second antigen binding molecule comprising i) a second target-binding domain, ii) a second cytokine receptor-binding domain, and iii) a Fc domain; wherein the first target-binding domain is capable of binding a first epitope on the target antigen and the second target-binding domain is capable of binding a second epitope on the target antigen, wherein the first and second target-binding domains do not compete for binding on the target antigen; and wherein the first cytokine receptor-binding domain is capable of binding a first cytokine receptor subunit and the second cytokine receptor-binding domain is capable of binding a second cytokine receptor subunit.

In one aspect, the first and second target-binding domains are antibody fragments, such as Fv, Fab, scFv, scFab molecules or single domain antibodies. In one aspect, the first and second target-binding domains are Fab molecules. In one aspect, the first target-binding domain comprises a heavy chain variable domain (VH₁), a light chain variable domain (VL₁), a heavy chain constant domain (CH1₁) and a light chain constant domain (CL₁), and wherein the second target-binding domain comprises a heavy chain variable domain (VH₂), a light chain variable domain (VL₂), a heavy chain constant domain (CH1₂) and a light chain constant domain (CL₂). In one aspect, the first and/or second target-binding domain is a cross-Fab molecule.

In one aspect, the first and second target-binding domains specifically bind to a tumor-associated antigen or a T-cell antigen. In one aspect, the first and second target-binding domains specifically bind to FAP, PD-1, Her2, Her3, LAG-3 or EGFR. In one aspect, a) the first target-binding domain comprises a VH₁ of SEQ ID NO: 20 and a VL₁ of SEQ ID NO: 21 and the second target-binding domain comprises a VH₂ of SEQ ID NO: 22 and a VL₂ of SEQ ID NO: 23, or b) the first target-binding domain comprises a VH₁ of SEQ ID NO: 22 and a VL₁ of SEQ ID NO: 23 and the second target-binding domain comprises a VH₂ of SEQ ID NO: 20 and a VL₂ of SEQ ID NO: 21, or c) the first target-binding domain comprises a VH₁ of SEQ ID NO: 76 and a VL₁ of SEQ ID NO: 77 and the second target-binding domain comprises a VH₂ of SEQ ID NO: 78 and a VL₂ of SEQ ID NO: 79, or d) the first target-binding domain comprises a VH₁ of SEQ ID NO: 78 and a VL₁ of SEQ ID NO: 79 and the second target-binding domain comprises a VH₂ of SEQ ID NO: 76 and a VL₂ of SEQ ID NO: 77; or e) the first target-binding domain comprises a VH₁ of SEQ ID NO: 93 and a VL₁ of SEQ ID NO: 94 and the second target-binding domain comprises a VH₂ of SEQ ID NO: 78 and a VL₂ of SEQ ID NO: 79; or f) the first target-binding domain comprises a VH₁ of SEQ ID NO: 78 and a VL₁ of SEQ ID NO: 79 and the second target-binding domains comprises a VH₂ of SEQ ID NO: 93 and a VL₂ of SEQ ID NO: 94, or g) the first target-binding domain comprises a VH₁ of SEQ ID NO: 130 and a VL₁ of SEQ ID NO: 131 and the second target-binding domain comprises a VH₂ of SEQ ID NO: 132 and a VL₂ of SEQ ID NO: 133; or h) the first target-binding domain comprises a VH₁ of SEQ ID NO: 132 and a VL₁ of SEQ ID NO: 133 and the second target-binding domains comprises a VH₂ of SEQ ID NO: 130 and a VL₂ of SEQ ID NO: 131; or i) the first target-binding domain comprises a VH₁ of SEQ ID NO: 140 and a VL₁ of SEQ ID NO: 141 and the second target-binding domain comprises a VH₂ of SEQ ID NO: 142 and a VL₂ of SEQ ID NO: 143; or j) the first target-binding domain comprises a VH₁ of SEQ ID NO: 142 and a VL₁ of SEQ ID NO: 143 and the second target-binding domains comprises a VH₂ of SEQ ID NO: 140 and a VL₂ of SEQ ID NO: 141; or k) the first target-binding domain comprises a VH₁ of SEQ ID NO: 114 and a VL₁ of SEQ ID NO: 115 and the second target-binding domain comprises a VH₂ of SEQ ID NO: 116 and a VL₂ of SEQ ID NO: 117; or l) the first target-binding domain comprises a VH₁ of SEQ ID NO: 116 and a VL₁ of SEQ ID NO: 117 and the second target-binding domains comprises a VH₂ of SEQ ID NO: 114 and a VL₂ of SEQ ID NO: 115; or m) the first target-binding domain comprises a VH₁ of SEQ ID NO: 150 and a VL₁ of SEQ ID NO: 151 and the second target-binding domain comprises a VH₂ of SEQ ID NO: 152 and a VL₂ of SEQ ID NO: 153; or n) the first target-binding domain comprises a VH₁ of SEQ ID NO: 152 and a VL₁ of SEQ ID NO: 153 and the second target-binding domains comprises a VH₂ of SEQ ID NO: 150 and a VL₂ of SEQ ID NO: 151.

In one aspect, both the first and second cytokine receptor subunits are subunits of the IFNγ receptor complex or, IL-2 receptor complex or IL-7 receptor complex. In one aspect, a) the first cytokine receptor-binding domain is capable of binding IFNγR1 and the second cytokine receptor-binding domain is capable of binding IFNγR2, or b) the first cytokine receptor-binding domain is capable of binding IFNγR2 and the second cytokine receptor-binding domain is capable of binding IFNγR1; or c) the first cytokine receptor-binding domain is capable of binding IL-2Rβ and the second cytokine receptor-binding domain is capable of binding IL-2Rγ, or d) the first cytokine receptor-binding domain is capable of binding IL-2Rγ and the second cytokine receptor-binding domain is capable of binding IL-2Rβ; or e) the first cytokine receptor-binding domain is capable of binding IL-2Rγ and the second cytokine receptor-binding domain is capable of binding IL-7Rα; or f) the first cytokine receptor-binding domain is capable of binding IL-7Rα and the second cytokine receptor-binding domain is capable of binding IL-2Rγ.

In one aspect, the first and second cytokine receptor-binding domains are antibody fragments, such as Fv, Fab, scFv, scFab or single domain antibodies. In one aspect, the first and second cytokine receptor-binding domains are VHH domains. In one aspect, a) the first cytokine receptor-binding domain comprises an amino acid sequence selected from SEQ ID NO: 1, SEQ ID NO: 2, SEQ ID NO: 3 and SEQ ID NO: 5 and the second cytokine receptor-binding domain comprises an amino acid sequence selected from SEQ ID NO: 6, SEQ ID NO: 7, SEQ ID NO: 8, SEQ ID NO: 9 and SEQ ID NO: 10, or b) the first cytokine receptor-binding domain comprises an amino acid sequence selected from SEQ ID NO: 6, SEQ ID NO: 7, SEQ ID NO: 8, SEQ ID NO: 9 and SEQ ID NO: 10 and the second cytokine receptor-binding domain comprises an amino acid sequence selected from SEQ ID NO: 1, SEQ ID NO: 2, SEQ ID NO: 3 and SEQ ID NO: 5; c) the first cytokine receptor-binding domain comprises an amino acid sequence selected from SEQ ID NO: 56, SEQ ID NO: 57, SEQ ID NO: 58 SEQ ID NO: 60, and the second cytokine receptor-binding domain comprises an amino acid sequence selected from SEQ ID NO: 62, SEQ ID NO: 63, SEQ ID NO: 64 and SEQ ID NO: 65; or d) the first cytokine receptor-binding domain comprises an amino acid sequence selected from SEQ ID NO: 62, SEQ ID NO: 63, SEQ ID NO: 64 and SEQ ID NO: 65 and the second cytokine receptor-binding domain comprises an amino acid sequence selected from SEQ ID NO: 56, SEQ ID NO: 57, SEQ ID NO: 58 SEQ ID NO: 60; or e) the first cytokine receptor-binding domain comprises an amino acid sequence selected from SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, and the second cytokine receptor-binding domain comprises an amino acid sequence selected from SEQ ID NO: 61, SEQ ID NO: 62, SEQ ID NO: 63, SEQ ID NO: 64, SEQ ID NO: 65; or f) the first cytokine receptor-binding domain comprises an amino acid sequence selected from SEQ ID NO: 61, SEQ ID NO: 62, SEQ ID NO: 63, SEQ ID NO: 64, SEQ ID NO: 65, and the second cytokine receptor-binding domain comprises an amino acid sequence selected from SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192.

In one aspect, a) the first cytokine receptor-binding domain comprises the sequence of SEQ ID NO: 3 and the second cytokine receptor-binding domain comprises the sequence of SEQ ID NO: 7, or b) the first cytokine receptor-binding domain comprises the sequence of SEQ ID NO: 7 and the second cytokine receptor-binding domain comprises the sequence of SEQ ID NO: 3; or c) the first cytokine receptor-binding domain comprises the sequence of SEQ ID NO: 60 and the second cytokine receptor-binding domain comprises the sequence of SEQ ID NO: 62, or d) the first cytokine receptor-binding domain comprises the sequence of SEQ ID NO: 62 and the second cytokine receptor-binding domain comprises the sequence of SEQ ID NO: 60; or e) the first cytokine receptor-binding domain comprises the sequence of SEQ ID NO: 186, and the second cytokine receptor-binding domain comprises the sequence of SEQ ID NO: 62, or f) the first cytokine receptor-binding domain comprises the sequence of SEQ ID NO: 62, and the second cytokine receptor-binding domain comprises the sequence of SEQ ID NO: 186.

In one aspect, the Fc domains of the first and second antigen binding molecules comprise a first Fc domain subunit and a second Fc domain subunit. In one aspect, the Fc domains of the first and second antigen binding molecules are IgG, particularly an IgG₁, Fc domains. In one aspect, the Fc domains of the first and second antigen binding molecules are human Fc domains. In one aspect, the Fc domains of the first and second antigen binding molecules comprise a modification promoting the association of the first and the second subunit of the Fc domains. In one aspect, the Fc domains of the first and second antigen binding molecules comprise one or more amino acid substitution that reduces binding to an Fc receptor and/or effector function.

In one aspect, the cytokine receptor-binding domains are fused via peptide linkers to their respective fusion points. In one aspect, the peptide linkers comprise an amino acid sequence selected from SEQ ID NO: 262, SEQ ID NO: 263, SEQ ID NO: 264, SEQ ID NO: 279, SEQ ID NO: 280 or SEQ ID NO: 281.

In one aspect, the first cytokine receptor-binding domain is fused at its C-terminus to the N-terminus of VH₁ or VL₁ of the first target-binding domain and the second cytokine receptor-binding domain is fused at its C-terminus to the N-terminus of VH₂ or VL₂ of the second target-binding domain. In one aspect, a) the first antigen binding molecule comprises a first polypeptide comprising a first Fc domain subunit, a second polypeptide comprising in order from the N-terminus to C-terminus the first cytokine receptor-binding domain, VH₁, CH1₁ and a second Fc domain subunit, and a third polypeptide comprising in order from the N-terminus to C-terminus VL₁ and CL₁; and the second antigen binding molecule comprises a first polypeptide comprising a first Fc domain subunit, a second polypeptide comprising in order from the N-terminus to C-terminus the second cytokine receptor-binding domain, VH₂, CH1₂ and a second Fc domain subunit, and a third polypeptide comprising in order from the N-terminus to C-terminus VL₂ and CL₂; or b) the first antigen binding molecule comprises a first polypeptide comprising a first Fc domain subunit, a second polypeptide comprising in order from the N-terminus to C-terminus VH₁, CH1₁ and a second Fc domain subunit, and a third polypeptide comprising in order from the N-terminus to C-terminus the first cytokine receptor-binding domain, VL₁ and CL₁; and the second antigen binding molecule comprises a first polypeptide comprising a first Fc domain subunit, a second polypeptide comprising in order from the N-terminus to C-terminus VH₂, CH1₂ and a second Fc domain subunit, a third polypeptide comprising in order from the N-terminus to C-terminus the second cytokine receptor-binding domain, VL₂ and CL₂; or c) the first antigen binding molecule comprises a first polypeptide comprising a first Fc domain subunit, a second polypeptide comprising in order from the N-terminus to C-terminus the first cytokine receptor-binding domain, VH₁, CH1₁ and a second Fc domain subunit, and a third polypeptide comprising in order from the N-terminus to C-terminus VL₁ and CL₁; and the second antigen binding molecule comprises a first polypeptide comprising a first Fc domain subunit, a second polypeptide comprising in order from the N-terminus to C-terminus VH₂, CH1₂ and a second Fc domain subunit, a third polypeptide comprising in order from the N-terminus to C-terminus the second cytokine receptor-binding domain, VL₂ and CL₂; wherein VH₁, VL₁, CH1₁ and CL₁ form the first target-binding domain and VH₂, VL₂, CH1₂, and CL₂, form the second target-binding domain.

In one aspect, a) the first antigen binding molecule comprises a first polypeptide comprising a first Fc domain subunit, a second polypeptide comprising in order from the N-terminus to C-terminus the first cytokine receptor-binding domain, VH₁, CH1₁ and a second Fc domain subunit, and a third polypeptide comprising in order from the N-terminus to C-terminus VL₁ and CL₁, and the second antigen binding molecule comprises a first polypeptide comprising a first Fc domain subunit, a second polypeptide comprising in order from the N-terminus to C-terminus the second cytokine receptor-binding domain, VH₂, CH1₂ and a second Fc domain subunit, and a third polypeptide comprising in order from the N-terminus to C-terminus VL₂ and CL₂; or b) the first antigen binding molecule comprises a first polypeptide comprising a first Fc domain subunit, a second polypeptide comprising in order from the N-terminus to C-terminus VH₁, CH1₁ and a second Fc domain subunit, and a third polypeptide comprising in order from the N-terminus to C-terminus the first cytokine receptor-binding domain, VL₁ and CL₁, and the second antigen binding molecule comprises a first polypeptide comprising a first Fc domain subunit, a second polypeptide comprising in order from the N-terminus to C-terminus VH₂, CH1₂ and a second Fc domain subunit, a third polypeptide comprising in order from the N-terminus to C-terminus the second cytokine receptor-binding domain, VL₂ and CL₂; or c) the first antigen binding molecule comprises a first polypeptide comprising a first Fc domain subunit, a second polypeptide comprising in order from the N-terminus to C-terminus the first cytokine receptor-binding domain, VH₁, CH1₁ and a second Fc domain subunit, and a third polypeptide comprising in order from the N-terminus to C-terminus VL₁ and CL₁, and the second antigen binding molecule comprises a first polypeptide comprising a first Fc domain subunit, a second polypeptide comprising in order from the N-terminus to C-terminus VH₂, CH1₂ and a second Fc domain subunit, a third polypeptide comprising in order from the N-terminus to C-terminus the second cytokine receptor-binding domain, VL₂ and CL₂; or d) the first antigen binding molecule comprises a first polypeptide comprising a first Fc domain subunit, a second polypeptide comprising in order from the N-terminus to C-terminus the first cytokine receptor-binding domain, VL₁, CH1₁ and a second Fc domain subunit, and a third polypeptide comprising in order from the N-terminus to C-terminus VH₁ and CL₁, and the second antigen binding molecule comprises a first polypeptide comprising a first Fc domain subunit, a second polypeptide comprising in order from the N-terminus to C-terminus the second cytokine receptor-binding domain, VH₂, CH1₂ and a second Fc domain subunit, and a third polypeptide comprising in order from the N-terminus to C-terminus VL₂ and CL₂; or e) the first antigen binding molecule comprises a first polypeptide comprising a first Fc domain subunit, a second polypeptide comprising in order from the N-terminus to C-terminus VL₁, CH1₁ and a second Fc domain subunit, and a third polypeptide comprising in order from the N-terminus to C-terminus the first cytokine receptor-binding domain, VH₁ and CL₁, and the second antigen binding molecule comprises a first polypeptide comprising a first Fc domain subunit, a second polypeptide comprising in order from the N-terminus to C-terminus VH₂, CH1₂ and a second Fc domain subunit, a third polypeptide comprising in order from the N-terminus to C-terminus the second cytokine receptor-binding domain, VL₂ and CL₂; or f) the first antigen binding molecule comprises a first polypeptide comprising a first Fc domain subunit, a second polypeptide comprising in order from the N-terminus to C-terminus the first cytokine receptor-binding domain, VL₁, CH1₁ and a second Fc domain subunit, and a third polypeptide comprising in order from the N-terminus to C-terminus VH₁ and CL₁, and the second antigen binding molecule comprises a first polypeptide comprising a first Fc domain subunit, a second polypeptide comprising in order from the N-terminus to C-terminus VH₂, CH1₂ and a second Fc domain subunit, and a third polypeptide comprising in order from the N-terminus to C-terminus the second cytokine receptor-binding domain, VL₂ and CL₂; or g) the first antigen binding molecule comprising a first polypeptide comprising a first Fc domain subunit, a second polypeptide comprising in order from the N-terminus to C-terminus a VL₁, CH1₁ and a second Fc domain subunit, and a third polypeptide comprising in order from the N-terminus to C-terminus a first cytokine receptor-binding domain, VH₁ and CL₁, and the second antigen binding molecule comprises a first polypeptide comprising a first Fc domain subunit, a second polypeptide comprising in order from the N-terminus to C-terminus a second cytokine receptor-binding domain, VH₂, CH1₂ and a second Fc domain subunit, and a third polypeptide comprising in order from the N-terminus to C-terminus VL₂ and CL₂; wherein VH₁, VL₁, CH1₁ and CL₁ form the first target-binding domain and VH₂, VL₂, CH1₂, and CL₂, form the second target-binding domain.

In one aspect, the first and the second target-binding domains specifically bind to FAP, and the first and second cytokine receptor subunits are subunits of the IFNγ receptor complex. Thus, In one aspect, a) the first antigen binding molecule comprises a first polypeptide comprising an amino acid sequence of SEQ ID NO: 28, a second polypeptide comprising an amino acid sequence of SEQ ID NO: 27 and a third polypeptide comprising an amino acid sequence of SEQ ID NO: 25, and the second antigen binding molecule comprises a first polypeptide comprising an amino acid sequence of SEQ ID NO: 28, a second polypeptide comprising an amino acid sequence of SEQ ID NO: 30 and a third polypeptide comprising an amino acid sequence of SEQ ID NO: 29; or b) the first antigen binding molecule comprises a first polypeptide comprising an amino acid sequence of SEQ ID NO: 28, a second polypeptide comprising an amino acid sequence of SEQ ID NO: 38 and a third polypeptide comprising an amino acid sequence of SEQ ID NO: 25, and the second antigen binding molecule comprises a first polypeptide comprising an amino acid sequence of SEQ ID NO: 28, a second polypeptide comprising an amino acid sequence of SEQ ID NO: 37 and a third polypeptide comprising an amino acid sequence of SEQ ID NO: 29; or c) the first antigen binding molecule comprises a first polypeptide comprising an amino acid sequence of SEQ ID NO: 28, a second polypeptide comprising an amino acid sequence of SEQ ID NO: 32 and a third polypeptide comprising an amino acid sequence of SEQ ID NO: 31, and the second antigen binding molecule comprises a first polypeptide comprising an amino acid sequence of SEQ ID NO: 28, a second polypeptide comprising an amino acid sequence of SEQ ID NO: 34 and a third polypeptide comprising an amino acid sequence of SEQ ID NO: 33; or d) the first antigen binding molecule comprises a first polypeptide comprising an amino acid sequence of SEQ ID NO: 28, a second polypeptide comprising an amino acid sequence of SEQ ID NO: 32 and a third polypeptide comprising an amino acid sequence of SEQ ID NO: 35, and the second antigen binding molecule comprises a first polypeptide comprising an amino acid sequence of SEQ ID NO: 28, a second polypeptide comprising an amino acid sequence of SEQ ID NO: 34 and a third polypeptide comprising an amino acid sequence of SEQ ID NO: 36; or e) the first antigen binding molecule comprises a first polypeptide comprising an amino acid sequence of SEQ ID NO: 28, a second polypeptide comprising an amino acid sequence of SEQ ID NO: 27 and a third polypeptide comprising an amino acid sequence of SEQ ID NO: 25, and the second antigen binding molecule comprises a first polypeptide comprising an amino acid sequence of SEQ ID NO: 28, a second polypeptide comprising an amino acid sequence of SEQ ID NO: 34 and a third polypeptide comprising an amino acid sequence of SEQ ID NO: 33; or f) the first antigen binding molecule comprises a first polypeptide comprising an amino acid sequence of SEQ ID NO: 28, a second polypeptide comprising an amino acid sequence of SEQ ID NO: 30 and a third polypeptide comprising an amino acid sequence of SEQ ID NO: 29, and the second antigen binding molecule comprises a first polypeptide comprising an amino acid sequence of SEQ ID NO: 28, a second polypeptide comprising an amino acid sequence of SEQ ID NO: 32 and a third polypeptide comprising an amino acid sequence of SEQ ID NO: 31; or g) the first antigen binding molecule comprising a first polypeptide comprises an amino acid sequence of SEQ ID NO: 28, a second polypeptide comprising an amino acid sequence of SEQ ID NO: 38 and a third polypeptide comprising an amino acid sequence of SEQ ID NO: 25, and the second antigen binding molecule comprises a first polypeptide comprising an amino acid sequence of SEQ ID NO: 28, a second polypeptide comprising an amino acid sequence of SEQ ID NO: 34 and a third polypeptide comprising an amino acid sequence of SEQ ID NO: 36; or h) the first antigen binding molecule comprises a first polypeptide comprising an amino acid sequence of SEQ ID NO: 28, a second polypeptide comprising an amino acid sequence of SEQ ID NO: 37 and a third polypeptide comprising an amino acid sequence of SEQ ID NO: 29, and the second antigen binding molecule comprises a first polypeptide comprising an amino acid sequence of SEQ ID NO: 28, a second polypeptide comprising an amino acid sequence of SEQ ID NO: 32 and a third polypeptide comprising an amino acid sequence of SEQ ID NO: 35.

In one aspect, first and the second target-binding domains specifically bind to EGFR, and the first and second cytokine receptor subunits are subunits of the IFNγ receptor complex. In one aspect, a) the first antigen binding molecule comprises a first polypeptide comprising an amino acid sequence of SEQ ID NO: 106, a second polypeptide comprising an amino acid sequence of SEQ ID NO: 260 and a third polypeptide comprising an amino acid sequence of SEQ ID NO: 124, and the second antigen binding molecule comprises a first polypeptide comprising an amino acid sequence of SEQ ID NO: 106, a second polypeptide comprising an amino acid sequence of SEQ ID NO: 259 and a third polypeptide comprising an amino acid sequence of SEQ ID NO: 127; or b) the first antigen binding molecule comprises a first polypeptide comprising an amino acid sequence of SEQ ID NO: 106, a second polypeptide comprising an amino acid sequence of SEQ ID NO: 119 and a third polypeptide comprising an amino acid sequence of SEQ ID NO: 256, and the second antigen binding molecule comprises a first polypeptide comprising an amino acid sequence of SEQ ID NO: 106, a second polypeptide comprising an amino acid sequence of SEQ ID NO: 122 and a third polypeptide comprising an amino acid sequence of SEQ ID NO: 255; or c) the first antigen binding molecule comprises a first polypeptide comprising an amino acid sequence of SEQ ID NO: 106, a second polypeptide comprising an amino acid sequence of SEQ ID NO: 261 and a third polypeptide comprising an amino acid sequence of SEQ ID NO: 124, and the second antigen binding molecule comprises a first polypeptide comprising an amino acid sequence of SEQ ID NO: 106, a second polypeptide comprising an amino acid sequence of SEQ ID NO: 258 and a third polypeptide comprising an amino acid sequence of SEQ ID NO: 127; or d) the first antigen binding molecule comprises a first polypeptide comprising an amino acid sequence of SEQ ID NO: 106, a second polypeptide comprising an amino acid sequence of SEQ ID NO: 119 and a third polypeptide comprising an amino acid sequence of SEQ ID NO: 257, and the second antigen binding molecule comprises a first polypeptide comprising an amino acid sequence of SEQ ID NO: 106, a second polypeptide comprising an amino acid sequence of SEQ ID NO: 112 and a third polypeptide comprising an amino acid sequence of SEQ ID NO: 254; or e) the first antigen binding molecule comprises a first polypeptide comprising an amino acid sequence of SEQ ID NO: 106, a second polypeptide comprising an amino acid sequence of SEQ ID NO: 260 and a third polypeptide comprising an amino acid sequence of SEQ ID NO: 124, and the second antigen binding molecule comprises a first polypeptide comprising an amino acid sequence of SEQ ID NO: 106, a second polypeptide comprising an amino acid sequence of SEQ ID NO: 112 and a third polypeptide comprising an amino acid sequence of SEQ ID NO: 255; or f) the first antigen binding molecule comprises a first polypeptide comprising an amino acid sequence of SEQ ID NO: 106, a second polypeptide comprising an amino acid sequence of SEQ ID NO: 119 and a third polypeptide comprising an amino acid sequence of SEQ ID NO: 256, and the second antigen binding molecule comprises a first polypeptide comprising an amino acid sequence of SEQ ID NO: 106, a second polypeptide comprising an amino acid sequence of SEQ ID NO: 259 and a third polypeptide comprising an amino acid sequence of SEQ ID NO: 127; or g) the first antigen binding molecule comprises a first polypeptide comprising an amino acid sequence of SEQ ID NO: 106, a second polypeptide comprising an amino acid sequence of SEQ ID NO: 261 and a third polypeptide comprising an amino acid sequence of SEQ ID NO: 124, and the second antigen binding molecule comprises a first polypeptide comprising an amino acid sequence of SEQ ID NO: 106, a second polypeptide comprising an amino acid sequence of SEQ ID NO: 112 and a third polypeptide comprising an amino acid sequence of SEQ ID NO: 254; or h) the first antigen binding molecule comprises a first polypeptide comprising an amino acid sequence of SEQ ID NO: 106, a second polypeptide comprising an amino acid sequence of SEQ ID NO: 119 and a third polypeptide comprising an amino acid sequence of SEQ ID NO: 257, and the second antigen binding molecule comprises a first polypeptide comprising an amino acid sequence of SEQ ID NO: 106, a second polypeptide comprising an amino acid sequence of SEQ ID NO: 258 and a third polypeptide comprising an amino acid sequence of SEQ ID NO: 127.

In one aspect, the first and the second target-binding domains specifically bind to PD-1, and the first and second cytokine receptor subunits are subunits of the IL-2 receptor complex. In one aspect, a) the first antigen binding molecule comprises a first polypeptide comprising an amino acid sequence of SEQ ID NO: 86, a second polypeptide comprising an amino acid sequence of SEQ ID NO: 44 and a third polypeptide comprising an amino acid sequence of SEQ ID NO: 83, and the second antigen binding molecule comprises a first polypeptide comprising an amino acid sequence of SEQ ID NO: 28, a second polypeptide comprising an amino acid sequence of SEQ ID NO: 88 and a third polypeptide comprising an amino acid sequence of SEQ ID NO: 87; or b) the first antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89, and the second antigen binding molecule comprises a first polypeptide comprising an amino acid sequence of SEQ ID NO: 92, a second polypeptide comprising an amino acid sequence of SEQ ID NO: 44 and a third polypeptide comprising an amino acid sequence of SEQ ID NO: 91; or c) the first antigen binding molecule comprises a first polypeptide comprising an amino acid sequence of SEQ ID NO: 28, a second polypeptide comprising an amino acid sequence of SEQ ID NO: 96 and a third polypeptide comprising an amino acid sequence of SEQ ID NO: 95, and the second antigen binding molecule comprises a first polypeptide comprising an amino acid sequence of SEQ ID NO: 28, a second polypeptide comprising an amino acid sequence of SEQ ID NO: 101 and a third polypeptide comprising an amino acid sequence of SEQ ID NO: 89; or d) the first antigen binding molecule comprises a first polypeptide comprising an amino acid sequence of SEQ ID NO: 28, a second polypeptide comprising an amino acid sequence of SEQ ID NO: 99 and a third polypeptide comprising an amino acid sequence of SEQ ID NO: 98, and the second antigen binding molecule comprises a first polypeptide comprising an amino acid sequence of SEQ ID NO: 28, a second polypeptide comprising an amino acid sequence of SEQ ID NO: 88 and a third polypeptide comprising an amino acid sequence of SEQ ID NO: 102; or e) the first antigen binding molecule comprises a first polypeptide comprising an amino acid sequence of SEQ ID NO: 28, a second polypeptide comprising an amino acid sequence of SEQ ID NO: 96 and a third polypeptide comprising an amino acid sequence of SEQ ID NO: 97, and the second antigen binding molecule comprises a first polypeptide comprising an amino acid sequence of SEQ ID NO: 28, a second polypeptide comprising an amino acid sequence of SEQ ID NO: 103 and a third polypeptide comprising an amino acid sequence of SEQ ID NO: 89; or f) the first antigen binding molecule comprises a first polypeptide comprising an amino acid sequence of SEQ ID NO: 28, a second polypeptide comprising an amino acid sequence of SEQ ID NO: 100 and a third polypeptide comprising an amino acid sequence of SEQ ID NO: 98, and the second antigen binding molecule comprises a first polypeptide comprising an amino acid sequence of SEQ ID NO: 28, a second polypeptide comprising an amino acid sequence of SEQ ID NO: 88 and a third polypeptide comprising an amino acid sequence of SEQ ID NO: 104; or g) the first antigen binding molecule comprises a first polypeptide comprising an amino acid sequence of SEQ ID NO: 28, a second polypeptide comprising an amino acid sequence of SEQ ID NO: 98 and a third polypeptide comprising an amino acid sequence of SEQ ID NO: 99, and the second antigen binding molecule comprises a first polypeptide comprising an amino acid sequence of SEQ ID NO: 28, a second polypeptide comprising an amino acid sequence of SEQ ID NO: 101 and a third polypeptide comprising an amino acid sequence of SEQ ID NO: 89; or h) the first antigen binding molecule comprises a first polypeptide comprising an amino acid sequence of SEQ ID NO: 28, a second polypeptide comprising an amino acid sequence of SEQ ID NO: 96 and a third polypeptide comprising an amino acid sequence of SEQ ID NO: 95, and the second antigen binding molecule comprises a first polypeptide comprising an amino acid sequence of SEQ ID NO: 28, a second polypeptide comprising an amino acid sequence of SEQ ID NO: 88 and a third polypeptide comprising an amino acid sequence of SEQ ID NO: 102; or i) the first antigen binding molecule comprises a first polypeptide comprising an amino acid sequence of SEQ ID NO: 28, a second polypeptide comprising an amino acid sequence of SEQ ID NO: 100 and a third polypeptide comprising an amino acid sequence of SEQ ID NO: 98, and the second antigen binding molecule comprises a first polypeptide comprising an amino acid sequence of SEQ ID NO: 28, a second polypeptide comprising an amino acid sequence of SEQ ID NO: 103 and a third polypeptide comprising an amino acid sequence of SEQ ID NO: 89; or j) the first antigen binding molecule comprises a first polypeptide comprising an amino acid sequence of SEQ ID NO: 28, a second polypeptide comprising an amino acid sequence of SEQ ID NO: 96 and a third polypeptide comprising an amino acid sequence of SEQ ID NO: 97, and the second antigen binding molecule comprises a first polypeptide comprising an amino acid sequence of SEQ ID NO: 28, a second polypeptide comprising an amino acid sequence of SEQ ID NO: 88 and a third polypeptide comprising an amino acid sequence of SEQ ID NO: 104.

In one aspect, the first and the second target-binding domains specifically bind to LAG-3, and the first and second cytokine receptor subunits are subunits of the IL-2 receptor complex. In one aspect, a) first antigen binding molecule comprises a first polypeptide comprising an amino acid sequence of SEQ ID NO: 106, a second polypeptide comprising an amino acid sequence of SEQ ID NO: 155 and a third polypeptide comprising an amino acid sequence of SEQ ID NO: 154, and the second antigen binding molecule comprises a first polypeptide comprising an amino acid sequence of SEQ ID NO: 106, a second polypeptide comprising an amino acid sequence of SEQ ID NO: 165 and a third polypeptide comprising an amino acid sequence of SEQ ID NO: 163.; or b) the first antigen binding molecule comprises a first polypeptide comprising an amino acid sequence of SEQ ID NO: 106, a second polypeptide comprising an amino acid sequence of SEQ ID NO: 161 and a third polypeptide comprising an amino acid sequence of SEQ ID NO: 160, and the second antigen binding molecule comprises a first polypeptide comprising an amino acid sequence of SEQ ID NO: 106, a second polypeptide comprising an amino acid sequence of SEQ ID NO: 158 and a third polypeptide comprising an amino acid sequence of SEQ ID NO: 159; or c) the first antigen binding molecule comprises a first polypeptide comprising an amino acid sequence of SEQ ID NO: 106, a second polypeptide comprising an amino acid sequence of SEQ ID NO: 155 and a third polypeptide comprising an amino acid sequence of SEQ ID NO: 156, and the second antigen binding molecule comprises a first polypeptide comprising an amino acid sequence of SEQ ID NO: 106, a second polypeptide comprising an amino acid sequence of SEQ ID NO: 164 and a third polypeptide comprising an amino acid sequence of SEQ ID NO: 163; or d) the first antigen binding molecule comprises a first polypeptide comprising an amino acid sequence of SEQ ID NO: 106, a second polypeptide comprising an amino acid sequence of SEQ ID NO: 162 and a third polypeptide comprising an amino acid sequence of SEQ ID NO: 160, and the second antigen binding molecule comprises a first polypeptide comprising an amino acid sequence of SEQ ID NO: 106, a second polypeptide comprising an amino acid sequence of SEQ ID NO: 158 and a third polypeptide comprising an amino acid sequence of SEQ ID NO: 157; or e) the first antigen binding molecule comprises a first polypeptide comprising an amino acid sequence of SEQ ID NO: 106, a second polypeptide comprising an amino acid sequence of SEQ ID NO: 161 and a third polypeptide comprising an amino acid sequence of SEQ ID NO: 160, and the second antigen binding molecule comprises a first polypeptide comprising an amino acid sequence of SEQ ID NO: 106, a second polypeptide comprising an amino acid sequence of SEQ ID NO: 165 and a third polypeptide comprising an amino acid sequence of SEQ ID NO: 163; or f) the first antigen binding molecule comprises a first polypeptide comprising an amino acid sequence of SEQ ID NO: 106, a second polypeptide comprising an amino acid sequence of SEQ ID NO: 155 and a third polypeptide comprising an amino acid sequence of SEQ ID NO: 154, and the second antigen binding molecule comprises a first polypeptide comprising an amino acid sequence of SEQ ID NO: 106, a second polypeptide comprising an amino acid sequence of SEQ ID NO: 158 and a third polypeptide comprising an amino acid sequence of SEQ ID NO: 159; or g) the first antigen binding molecule comprises a first polypeptide comprising an amino acid sequence of SEQ ID NO: 106, a second polypeptide comprising an amino acid sequence of SEQ ID NO: 162 and a third polypeptide comprising an amino acid sequence of SEQ ID NO: 160, and the second antigen binding molecule comprises a first polypeptide comprising an amino acid sequence of SEQ ID NO: 106, a second polypeptide comprising an amino acid sequence of SEQ ID NO: 164 and a third polypeptide comprising an amino acid sequence of SEQ ID NO: 163; or h) the first antigen binding molecule comprises a first polypeptide comprising an amino acid sequence of SEQ ID NO: 106, a second polypeptide comprising an amino acid sequence of SEQ ID NO: 155 and a third polypeptide comprising an amino acid sequence of SEQ ID NO: 156, and the second antigen binding molecule comprises a first polypeptide comprising an amino acid sequence of SEQ ID NO: 106, a second polypeptide comprising an amino acid sequence of SEQ ID NO: 158 and a third polypeptide comprising an amino acid sequence of SEQ ID NO: 157.

In one aspect, the first and the second target-binding domains specifically bind to EGFR, and the first and second cytokine receptor subunits are subunits of the IL-2 receptor complex. In one aspect, a) first antigen binding molecule comprises a first polypeptide comprising an amino acid sequence of SEQ ID NO: 106, a second polypeptide comprising an amino acid sequence of SEQ ID NO: 155 and a third polypeptide comprising an amino acid sequence of SEQ ID NO: 154, and the second antigen binding molecule comprises a first polypeptide comprising an amino acid sequence of SEQ ID NO: 106, a second polypeptide comprising an amino acid sequence of SEQ ID NO: 165 and a third polypeptide comprising an amino acid sequence of SEQ ID NO: 163; or b) the first antigen binding molecule comprises a first polypeptide comprising an amino acid sequence of SEQ ID NO: 106, a second polypeptide comprising an amino acid sequence of SEQ ID NO: 161 and a third polypeptide comprising an amino acid sequence of SEQ ID NO: 160, and the second antigen binding molecule comprises a first polypeptide comprising an amino acid sequence of SEQ ID NO: 106, a second polypeptide comprising an amino acid sequence of SEQ ID NO: 158 and a third polypeptide comprising an amino acid sequence of SEQ ID NO: 159.; or c) the first antigen binding molecule comprises a first polypeptide comprising an amino acid sequence of SEQ ID NO: 106, a second polypeptide comprising an amino acid sequence of SEQ ID NO: 155 and a third polypeptide comprising an amino acid sequence of SEQ ID NO: 156, and the second antigen binding molecule comprises a first polypeptide comprising an amino acid sequence of SEQ ID NO: 106, a second polypeptide comprising an amino acid sequence of SEQ ID NO: 164 and a third polypeptide comprising an amino acid sequence of SEQ ID NO: 163; or d) the first antigen binding molecule comprises a first polypeptide comprising an amino acid sequence of SEQ ID NO: 106, a second polypeptide comprising an amino acid sequence of SEQ ID NO: 162 and a third polypeptide comprising an amino acid sequence of SEQ ID NO: 160, and the second antigen binding molecule comprises a first polypeptide comprising an amino acid sequence of SEQ ID NO: 106, a second polypeptide comprising an amino acid sequence of SEQ ID NO: 158 and a third polypeptide comprising an amino acid sequence of SEQ ID NO: 157; or e) the first antigen binding molecule comprises a first polypeptide comprising an amino acid sequence of SEQ ID NO: 106, a second polypeptide comprising an amino acid sequence of SEQ ID NO: 161 and a third polypeptide comprising an amino acid sequence of SEQ ID NO: 160, and the second antigen binding molecule comprises a first polypeptide comprising an amino acid sequence of SEQ ID NO: 106, a second polypeptide comprising an amino acid sequence of SEQ ID NO: 165 and a third polypeptide comprising an amino acid sequence of SEQ ID NO: 163; or f) the first antigen binding molecule comprises a first polypeptide comprising an amino acid sequence of SEQ ID NO: 106, a second polypeptide comprising an amino acid sequence of SEQ ID NO: 155 and a third polypeptide comprising an amino acid sequence of SEQ ID NO: 154, and the second antigen binding molecule comprises a first polypeptide comprising an amino acid sequence of SEQ ID NO: 106, a second polypeptide comprising an amino acid sequence of SEQ ID NO: 158 and a third polypeptide comprising an amino acid sequence of SEQ ID NO: 159; or g) the first antigen binding molecule comprises a first polypeptide comprising an amino acid sequence of SEQ ID NO: 106, a second polypeptide comprising an amino acid sequence of SEQ ID NO: 162 and a third polypeptide comprising an amino acid sequence of SEQ ID NO: 160, and the second antigen binding molecule comprises a first polypeptide comprising an amino acid sequence of SEQ ID NO: 106, a second polypeptide comprising an amino acid sequence of SEQ ID NO: 164 and a third polypeptide comprising an amino acid sequence of SEQ ID NO: 163; or h) the first antigen binding molecule comprises a first polypeptide comprising an amino acid sequence of SEQ ID NO: 106, a second polypeptide comprising an amino acid sequence of SEQ ID NO: 155 and a third polypeptide comprising an amino acid sequence of SEQ ID NO: 156, and the second antigen binding molecule comprises a first polypeptide comprising an amino acid sequence of SEQ ID NO: 106, a second polypeptide comprising an amino acid sequence of SEQ ID NO: 158 and a third polypeptide comprising an amino acid sequence of SEQ ID NO: 157.

In one aspect, the first and the second target-binding domains specifically bind to FAP, and the first and second cytokine receptor subunits are subunits of the IL-2 receptor complex. In one aspect, a) the first antigen binding molecule comprises a first polypeptide comprising an amino acid sequence of SEQ ID NO: 106, a second polypeptide comprising an amino acid sequence of SEQ ID NO: 110 and a third polypeptide comprising an amino acid sequence of SEQ ID NO: 25, and the second antigen binding molecule comprises a first polypeptide comprising an amino acid sequence of SEQ ID NO: 106, a second polypeptide comprising an amino acid sequence of SEQ ID NO: 113 and a third polypeptide comprising an amino acid sequence of SEQ ID NO: 29; or b) the first antigen binding molecule comprises a first polypeptide comprising an amino acid sequence of SEQ ID NO: 106, a second polypeptide comprising an amino acid sequence of SEQ ID NO: 32 and a third polypeptide comprising an amino acid sequence of SEQ ID NO: 105, and the second antigen binding molecule comprises a first polypeptide comprising an amino acid sequence of SEQ ID NO: 106, a second polypeptide comprising an amino acid sequence of SEQ ID NO: 34 and a third polypeptide comprising an amino acid sequence of SEQ ID NO: 109; or c) the first antigen binding molecule comprises a first polypeptide comprising an amino acid sequence of SEQ ID NO: 106, a second polypeptide comprising an amino acid sequence of SEQ ID NO: 111 and a third polypeptide comprising an amino acid sequence of SEQ ID NO: 25, and the second antigen binding molecule comprises a first polypeptide comprising an amino acid sequence of SEQ ID NO: 106, a second polypeptide comprising an amino acid sequence of SEQ ID NO: 112 and a third polypeptide comprising an amino acid sequence of SEQ ID NO: 29; or d) the first antigen binding molecule comprises a first polypeptide comprising an amino acid sequence of SEQ ID NO: 106, a second polypeptide comprising an amino acid sequence of SEQ ID NO: 32 and a third polypeptide comprising an amino acid sequence of SEQ ID NO: 107, and the second antigen binding molecule comprises a first polypeptide comprising an amino acid sequence of SEQ ID NO: 106, a second polypeptide comprising an amino acid sequence of SEQ ID NO: 34 and a third polypeptide comprising an amino acid sequence of SEQ ID NO: 108; or e) the first antigen binding molecule comprises a first polypeptide comprising an amino acid sequence of SEQ ID NO: 106, a second polypeptide comprising an amino acid sequence of SEQ ID NO: 110 and a third polypeptide comprising an amino acid sequence of SEQ ID NO: 25, and the second antigen binding molecule comprises a first polypeptide comprising an amino acid sequence of SEQ ID NO: 106, a second polypeptide comprising an amino acid sequence of SEQ ID NO: 34 and a third polypeptide comprising an amino acid sequence of SEQ ID NO: 109; or f) the first antigen binding molecule comprises a first polypeptide comprising an amino acid sequence of SEQ ID NO: 106, a second polypeptide comprising an amino acid sequence of SEQ ID NO: 32 and a third polypeptide comprising an amino acid sequence of SEQ ID NO: 105, and the second antigen binding molecule comprises a first polypeptide comprising an amino acid sequence of SEQ ID NO: 106, a second polypeptide comprising an amino acid sequence of SEQ ID NO: 113 and a third polypeptide comprising an amino acid sequence of SEQ ID NO: 29; or g) the first antigen binding molecule comprises a first polypeptide comprising an amino acid sequence of SEQ ID NO: 106, a second polypeptide comprising an amino acid sequence of SEQ ID NO: 111 and a third polypeptide comprising an amino acid sequence of SEQ ID NO: 25, and the second antigen binding molecule comprises a first polypeptide comprising an amino acid sequence of SEQ ID NO: 106, a second polypeptide comprising an amino acid sequence of SEQ ID NO: 34 and a third polypeptide comprising an amino acid sequence of SEQ ID NO: 108; or h) the first antigen binding molecule comprises a first polypeptide comprising an amino acid sequence of SEQ ID NO: 106, a second polypeptide comprising an amino acid sequence of SEQ ID NO: 32 and a third polypeptide comprising an amino acid sequence of SEQ ID NO: 107, and the second antigen binding molecule comprises a first polypeptide comprising an amino acid sequence of SEQ ID NO: 106, a second polypeptide comprising an amino acid sequence of SEQ ID NO: 112 and a third polypeptide comprising an amino acid sequence of SEQ ID NO: 29.

In one aspect, the first and the second target-binding domains specifically bind to Her2, and the first and second cytokine receptor subunits are subunits of the IL-2 receptor complex. In one aspect, a) first antigen binding molecule comprises a first polypeptide comprising an amino acid sequence of SEQ ID NO: 106, a second polypeptide comprising an amino acid sequence of SEQ ID NO: 136 and a third polypeptide comprising an amino acid sequence of SEQ ID NO: 134, and the second antigen binding molecule comprises a first polypeptide comprising an amino acid sequence of SEQ ID NO: 106, a second polypeptide comprising an amino acid sequence of SEQ ID NO: 138 and a third polypeptide comprising an amino acid sequence of SEQ ID NO: 137; or b) the first antigen binding molecule comprises a first polypeptide comprising an amino acid sequence of SEQ ID NO: 106, a second polypeptide comprising an amino acid sequence of SEQ ID NO: 135 and a third polypeptide comprising an amino acid sequence of SEQ ID NO: 134, and the second antigen binding molecule comprises a first polypeptide comprising an amino acid sequence of SEQ ID NO: 106, a second polypeptide comprising an amino acid sequence of SEQ ID NO: 138 and a third polypeptide comprising an amino acid sequence of SEQ ID NO: 139.

In one aspect, the first and the second target-binding domains specifically bind to Her3, and the first and second cytokine receptor subunits are subunits of the IL-2 receptor complex. In one aspect, a) the first antigen binding molecule comprises a first polypeptide comprising an amino acid sequence of SEQ ID NO: 106, a second polypeptide comprising an amino acid sequence of SEQ ID NO: 146 and a third polypeptide comprising an amino acid sequence of SEQ ID NO: 144, and the second antigen binding molecule comprises a first polypeptide comprising an amino acid sequence of SEQ ID NO: 106, a second polypeptide comprising an amino acid sequence of SEQ ID NO: 148 and a third polypeptide comprising an amino acid sequence of SEQ ID NO: 147; or b) the first antigen binding molecule comprises a first polypeptide comprising an amino acid sequence of SEQ ID NO: 106, a second polypeptide comprising an amino acid sequence of SEQ ID NO: 145 and a third polypeptide comprising an amino acid sequence of SEQ ID NO: 144, and the second antigen binding molecule comprises a first polypeptide comprising an amino acid sequence of SEQ ID NO: 106, a second polypeptide comprising an amino acid sequence of SEQ ID NO: 148 and a third polypeptide comprising an amino acid sequence of SEQ ID NO: 149.

In one aspect, the first and the second target-binding domains specifically bind to PD-1, and the first and second cytokine receptor subunits are subunits of the IL-7 receptor complex. In one aspect, a) the first antigen binding molecule comprises a first polypeptide comprising an amino acid sequence of SEQ ID NO: 106, a second polypeptide comprising an amino acid sequence of SEQ ID NO: 194 and a third polypeptide comprising an amino acid sequence of SEQ ID NO: 193, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89; or b) the first antigen binding molecule comprises a first polypeptide comprising an amino acid sequence of SEQ ID NO: 106, a second polypeptide comprising an amino acid sequence of SEQ ID NO: 195 and a third polypeptide comprising an amino acid sequence of SEQ ID NO: 124, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89; or c) the first antigen binding molecule comprises a first polypeptide comprising an amino acid sequence of SEQ ID NO: 106, a second polypeptide comprising an amino acid sequence of SEQ ID NO: 196 and a third polypeptide comprising an amino acid sequence of SEQ ID NO: 124, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89; or d) the first antigen binding molecule comprises a first polypeptide comprising an amino acid sequence of SEQ ID NO: 106, a second polypeptide comprising an amino acid sequence of SEQ ID NO: 197 and a third polypeptide comprising an amino acid sequence of SEQ ID NO: 124, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89; or d) the first antigen binding molecule comprises a first polypeptide comprising an amino acid sequence of SEQ ID NO: 106, a second polypeptide comprising an amino acid sequence of SEQ ID NO: 198 and a third polypeptide comprising an amino acid sequence of SEQ ID NO: 124, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89; or e) the first antigen binding molecule comprises a first polypeptide comprising an amino acid sequence of SEQ ID NO: 106, a second polypeptide comprising an amino acid sequence of SEQ ID NO: 199 and a third polypeptide comprising an amino acid sequence of SEQ ID NO: 124, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89; or f) the first antigen binding molecule comprises a first polypeptide comprising an amino acid sequence of SEQ ID NO: 106, a second polypeptide comprising an amino acid sequence of SEQ ID NO: 200 and a third polypeptide comprising an amino acid sequence of SEQ ID NO: 124, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89; or g) the first antigen binding molecule comprises a first polypeptide comprising an amino acid sequence of SEQ ID NO: 106, a second polypeptide comprising an amino acid sequence of SEQ ID NO: 201 and a third polypeptide comprising an amino acid sequence of SEQ ID NO: 124, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89; or h) the first antigen binding molecule comprises a first polypeptide comprising an amino acid sequence of SEQ ID NO: 106, a second polypeptide comprising an amino acid sequence of SEQ ID NO: 202 and a third polypeptide comprising an amino acid sequence of SEQ ID NO: 124, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89; or i) the first antigen binding molecule comprises a first polypeptide comprising an amino acid sequence of SEQ ID NO: 106, a second polypeptide comprising an amino acid sequence of SEQ ID NO: 203 and a third polypeptide comprising an amino acid sequence of SEQ ID NO: 124, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89; or j) the first antigen binding molecule comprises a first polypeptide comprising an amino acid sequence of SEQ ID NO: 106, a second polypeptide comprising an amino acid sequence of SEQ ID NO: 204 and a third polypeptide comprising an amino acid sequence of SEQ ID NO: 124, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89; or k) the first antigen binding molecule comprises a first polypeptide comprising an amino acid sequence of SEQ ID NO: 106, a second polypeptide comprising an amino acid sequence of SEQ ID NO: 205 and a third polypeptide comprising an amino acid sequence of SEQ ID NO: 124, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89.

In one aspect, the first target-binding domain is fused at its C-terminus of CH1₁ to the N-terminus of the first Fc domain subunit and the first cytokine receptor-binding domain is fused at its C-terminus to the N-terminus of the second Fc domain subunit and the second target-binding domain is fused at its C-terminus of CH1₂ to the N-terminus of the first Fc domain subunit and the second cytokine receptor-binding domain is fused at its C-terminus to the N-terminus of the second Fc domain subunit. In one aspect, a) the first antigen binding molecule comprises a first polypeptide comprising in order from the N-terminus to C-terminus VH₁, CH1₁ and a first Fc domain subunit, a second polypeptide comprising in order from the N-terminus to C-terminus VL₁ and CL₁, and a third polypeptide comprising in order from the N-terminus to C-terminus the first cytokine receptor-binding domain and a second Fc domain subunit, and the second antigen binding molecule comprises a first polypeptide comprising in order from the N-terminus to C-terminus VH₂, CH1₂ and a first Fc domain subunit, a second polypeptide comprising in order from the N-terminus to C-terminus VL₂ and CL₂, and a third polypeptide comprising in order from the N-terminus to C-terminus the second cytokine receptor-binding domain and a second Fc domain subunit; or b) the first antigen binding molecule comprises a first polypeptide comprising in order from the N-terminus to C-terminus VL₁, CH1₁ and a first Fc domain subunit, a second polypeptide comprising in order from the N-terminus to C-terminus VH₁ and CL₁, and a third polypeptide comprising in order from the N-terminus to C-terminus the first cytokine receptor-binding domain and a second Fc domain subunit, and the second antigen binding molecule comprises a first polypeptide comprising in order from the N-terminus to C-terminus VH₂, CH1₂ and a first Fc domain subunit, a second polypeptide comprising in order from the N-terminus to C-terminus VL₂ and CL₂, and a third polypeptide comprising in order from the N-terminus to C-terminus the second cytokine receptor-binding domain and a second Fc domain subunit; wherein VH₁, VL₁, CH1₁ and CL₁ form the first target-binding domain and VH₂, VL₂, CH1₂, and CL₂, form the second target-binding domain. In one aspect, a) the first antigen binding molecule comprises a first polypeptide comprising an amino acid sequence of SEQ ID NO: 32, a second polypeptide comprising an amino acid sequence of SEQ ID NO: 25 and a third polypeptide comprising an amino acid sequence of SEQ ID NO: 39, and the second antigen binding molecule comprises a first polypeptide comprising an amino acid sequence of SEQ ID NO: 34, a second polypeptide comprising an amino acid sequence of SEQ ID NO: 29 and a third polypeptide comprising an amino acid sequence of SEQ ID NO: 40; or b) the first antigen binding molecule comprises a first polypeptide comprising an amino acid sequence of SEQ ID NO: 34, a second polypeptide comprising an amino acid sequence of SEQ ID NO: 29 and a third polypeptide comprising an amino acid sequence of SEQ ID NO: 39, and the second antigen binding molecule comprises a first polypeptide comprising an amino acid sequence of SEQ ID NO: 32, a second polypeptide comprising an amino acid sequence of SEQ ID NO: 25 and a third polypeptide comprising an amino acid sequence of SEQ ID NO: 40; or c) the first antigen binding molecule comprises a first polypeptide comprising an amino acid sequence of SEQ ID NO: 237, a second polypeptide comprising an amino acid sequence of SEQ ID NO: 32 and a third polypeptide comprising an amino acid sequence of SEQ ID NO: 25, and the second antigen binding molecule comprises a first polypeptide comprising an amino acid sequence of SEQ ID NO: 238, a second polypeptide comprising an amino acid sequence of SEQ ID NO: 34 and a third polypeptide comprising an amino acid sequence of SEQ ID NO: 29; or d) the first antigen binding molecule comprises a first polypeptide comprising an amino acid sequence of SEQ ID NO: 235, a second polypeptide comprising an amino acid sequence of SEQ ID NO: 32 and a third polypeptide comprising an amino acid sequence of SEQ ID NO: 25, and the second antigen binding molecule comprises a first polypeptide comprising an amino acid sequence of SEQ ID NO: 236, a second polypeptide comprising an amino acid sequence of SEQ ID NO: 34 and a third polypeptide comprising an amino acid sequence of SEQ ID NO: 29; or e) the first antigen binding molecule comprises a first polypeptide comprising an amino acid sequence of SEQ ID NO: 239, a second polypeptide comprising an amino acid sequence of SEQ ID NO: 32 and a third polypeptide comprising an amino acid sequence of SEQ ID NO: 25, and the second antigen binding molecule comprises a first polypeptide comprising an amino acid sequence of SEQ ID NO: 240, a second polypeptide comprising an amino acid sequence of SEQ ID NO: 34 and a third polypeptide comprising an amino acid sequence of SEQ ID NO: 29; or f) the first antigen binding molecule comprises a first polypeptide comprising an amino acid sequence of SEQ ID NO: 235, a second polypeptide comprising an amino acid sequence of SEQ ID NO: 32 and a third polypeptide comprising an amino acid sequence of SEQ ID NO: 25, and the second antigen binding molecule comprises a first polypeptide comprising an amino acid sequence of SEQ ID NO: 237, a second polypeptide comprising an amino acid sequence of SEQ ID NO: 34 and a third polypeptide comprising an amino acid sequence of SEQ ID NO: 29; or g) the first antigen binding molecule comprises a first polypeptide comprising an amino acid sequence of SEQ ID NO: 236, a second polypeptide comprising an amino acid sequence of SEQ ID NO: 32 and a third polypeptide comprising an amino acid sequence of SEQ ID NO: 25, and the second antigen binding molecule comprises a first polypeptide comprising an amino acid sequence of SEQ ID NO: 235, a second polypeptide comprising an amino acid sequence of SEQ ID NO: 34 and a third polypeptide comprising an amino acid sequence of SEQ ID NO: 29; or h) the first antigen binding molecule comprises a first polypeptide comprising an amino acid sequence of SEQ ID NO: 240, a second polypeptide comprising an amino acid sequence of SEQ ID NO: 32 and a third polypeptide comprising an amino acid sequence of SEQ ID NO: 25, and the second antigen binding molecule comprises a first polypeptide comprising an amino acid sequence of SEQ ID NO: 239, a second polypeptide comprising an amino acid sequence of SEQ ID NO: 34 and a third polypeptide comprising an amino acid sequence of SEQ ID NO: 29.

In one aspect, the first target-binding domain is fused at its C-terminus of CH1₁ to the N-terminus of one of the Fc domain subunits and the first cytokine receptor-binding domain is fused at its N-terminus to the C-terminus of the same Fc domain subunit and the second target-binding domain is fused at its C-terminus of CH1₂ to the N-terminus of one of the Fc domain subunits and the second cytokine receptor-binding domain is fused at its N-terminus to the C-terminus of the same Fc domain subunit. In one aspect, a) the first antigen binding molecule comprises a first polypeptide comprising a first Fc domain subunit, a second polypeptide comprising in order from the N-terminus to C-terminus a VH₁, a CH1₁, a second Fc domain subunit and a the first cytokine receptor-binding domain, and a third polypeptide comprising in order from the N-terminus to C-terminus a VL₁ and a CL₁; and the second antigen binding molecule comprises a first polypeptide comprising a first Fc domain subunit, a second polypeptide comprising in order from the N-terminus to C-terminus a VH₂, a CH1₂, a second Fc domain subunit and a the second cytokine receptor-binding domain, and a third polypeptide comprising in order from the N-terminus to C-terminus VL₂ and CL₂; or b) the first antigen binding molecule comprises a first polypeptide comprising a first Fc domain subunit, a second polypeptide comprising in order from the N-terminus to C-terminus a VL₁, a CH1₁, a second Fc domain subunit and a the first cytokine receptor-binding domain, and a third polypeptide comprising in order from the N-terminus to C-terminus a VH₁ and a CL₁; and the second antigen binding molecule comprises a first polypeptide comprising a first Fc domain subunit, a second polypeptide comprising in order from the N-terminus to C-terminus a VH₂, a CH1₂, a second Fc domain subunit and the second cytokine receptor-binding domain, and a third polypeptide comprising in order from the N-terminus to C-terminus a VL₂ and a CL₂; or c) the first antigen binding molecule comprises a first polypeptide comprising a first Fc domain subunit, a second polypeptide comprising in order from the N-terminus to C-terminus a VL₁, a CH1₁, a second Fc domain subunit and a the first cytokine receptor-binding domain, and a third polypeptide comprising in order from the N-terminus to C-terminus a VH₁ and a CL₁; and the second antigen binding molecule comprises a first polypeptide comprising a first Fc domain subunit, a second polypeptide comprising in order from the N-terminus to C-terminus a VH₂, a CH1₂, a second Fc domain subunit and a the second cytokine receptor-binding domain, and a third polypeptide comprising in order from the N-terminus to C-terminus VL₂ and CL₂; wherein VH₁, VL₁, CH1₁ and CL₁ form the first target-binding domain and VH₂, VL₂, CH1₂, and CL₂, form the second target-binding domain. In one aspect, a) the first antigen binding molecule comprises a first polypeptide comprising an amino acid sequence of SEQ ID NO: 28, a second polypeptide comprising an amino acid sequence of SEQ ID NO: 42 and a third polypeptide comprising an amino acid sequence of SEQ ID NO: 41, and the second antigen binding molecule comprises a first polypeptide comprising an amino acid sequence of SEQ ID NO: 45, a second polypeptide comprising an amino acid sequence of SEQ ID NO: 44 and a third polypeptide comprising an amino acid sequence of SEQ ID NO: 43; or b) the first antigen binding molecule comprises a first polypeptide comprising an amino acid sequence of SEQ ID NO: 28, a second polypeptide comprising an amino acid sequence of SEQ ID NO: 46 and a third polypeptide comprising an amino acid sequence of SEQ ID NO: 41, and the second antigen binding molecule comprises a first polypeptide comprising an amino acid sequence of SEQ ID NO: 47, a second polypeptide comprising an amino acid sequence of SEQ ID NO: 44 and a third polypeptide comprising an amino acid sequence of SEQ ID NO: 43; or c) the first antigen binding molecule comprises a first polypeptide comprising an amino acid sequence of SEQ ID NO: 241, a second polypeptide comprising an amino acid sequence of SEQ ID NO: 246 and a third polypeptide comprising an amino acid sequence of SEQ ID NO: 25, and the second antigen binding molecule comprises a first polypeptide comprising an amino acid sequence of SEQ ID NO: 241, a second polypeptide comprising an amino acid sequence of SEQ ID NO: 249 and a third polypeptide comprising an amino acid sequence of SEQ ID NO: 29; or d) the first antigen binding molecule comprises a first polypeptide comprising an amino acid sequence of SEQ ID NO: 241, a second polypeptide comprising an amino acid sequence of SEQ ID NO: 242 and a third polypeptide comprising an amino acid sequence of SEQ ID NO: 25, and the second antigen binding molecule comprises a first polypeptide comprising an amino acid sequence of SEQ ID NO: 241, a second polypeptide comprising an amino acid sequence of SEQ ID NO: 245 and a third polypeptide comprising an amino acid sequence of SEQ ID NO: 29; or e) the first antigen binding molecule comprising a first polypeptide comprises an amino acid sequence of SEQ ID NO: 241, a second polypeptide comprising an amino acid sequence of SEQ ID NO: 250 and a third polypeptide comprising an amino acid sequence of SEQ ID NO: 25, and the second antigen binding molecule comprises a first polypeptide comprising an amino acid sequence of SEQ ID NO: 241, a second polypeptide comprising an amino acid sequence of SEQ ID NO: 253 and a third polypeptide comprising an amino acid sequence of SEQ ID NO: 29; or f) the first antigen binding molecule comprises a first polypeptide comprising an amino acid sequence of SEQ ID NO: 241, a second polypeptide comprising an amino acid sequence of SEQ ID NO: 248 and a third polypeptide comprising an amino acid sequence of SEQ ID NO: 25, and the second antigen binding molecule comprises a first polypeptide comprising an amino acid sequence of SEQ ID NO: 241, a second polypeptide comprising an amino acid sequence of SEQ ID NO: 247 and a third polypeptide comprising an amino acid sequence of SEQ ID NO: 29; or g) the first antigen binding molecule comprises a first polypeptide comprising an amino acid sequence of SEQ ID NO: 241, a second polypeptide comprising an amino acid sequence of SEQ ID NO: 244 and a third polypeptide comprising an amino acid sequence of SEQ ID NO: 25, and the second antigen binding molecule comprises a first polypeptide comprising an amino acid sequence of SEQ ID NO: 241, a second polypeptide comprising an amino acid sequence of SEQ ID NO: 243 and a third polypeptide comprising an amino acid sequence of SEQ ID NO: 29; or h) the first antigen binding molecule comprises a first polypeptide comprising an amino acid sequence of SEQ ID NO: 241, a second polypeptide comprising an amino acid sequence of SEQ ID NO: 252 and a third polypeptide comprising an amino acid sequence of SEQ ID NO: 25, and the second antigen binding molecule comprises a first polypeptide comprising an amino acid sequence of SEQ ID NO: 241, a second polypeptide comprising an amino acid sequence of SEQ ID NO: 251 and a third polypeptide comprising an amino acid sequence of SEQ ID NO: 29; or i) the first antigen binding molecule comprises a first polypeptide comprising an amino acid sequence of SEQ ID NO: 28, a second polypeptide comprising an amino acid sequence of SEQ ID NO: 42 and a third polypeptide comprising an amino acid sequence of SEQ ID NO: 42, and the second antigen binding molecule comprises a first polypeptide comprising an amino acid sequence of SEQ ID NO: 241, a second polypeptide comprising an amino acid sequence of SEQ ID NO: 245 and a third polypeptide comprising an amino acid sequence of SEQ ID NO: 29.

In one aspect, the first and second cytokine receptor-binding domains of the pair of antigen binding molecules are Fab molecules. In one aspect, a) the first cytokine receptor-binding domain comprises a VH domain comprising the amino acid sequence of SEQ ID NO: 282 and a VL domain comprising the amino acid sequence of SEQ ID NO: 283 or VH domain comprising the amino acid sequence of SEQ ID NO: 284 and a VL domain comprising the amino acid sequence of SEQ ID NO: 285, and the second cytokine receptor domain comprises a VH domain comprising the amino acid sequence of SEQ ID NO: 286 and a VL domain comprising the amino acid sequence of SEQ ID NO: 287 or VH domain comprising the amino acid sequence of SEQ ID NO: 288 and a VL domain comprising the amino acid sequence of SEQ ID NO: 289; or b) the first cytokine receptor-binding domain comprises a VH domain comprising the amino acid sequence of SEQ ID NO: 286 and a VL domain comprising the amino acid sequence of SEQ ID NO: 287 or VH domain comprising the amino acid sequence of SEQ ID NO: 288 and a VL domain comprising the amino acid sequence of SEQ ID NO: 289, and the second cytokine receptor domain comprises a VH domain comprising the amino acid sequence of SEQ ID NO: 282 and a VL domain comprising the amino acid sequence of SEQ ID NO: 283 or VH domain comprising the amino acid sequence of SEQ ID NO: 284 and a VL domain comprising the amino acid sequence of SEQ ID NO: 285.

In one aspect, the first and second cytokine receptor-binding domains of the pair of antigen binding molecules are scFv molecules. In one aspect, a) the first cytokine receptor-binding domain comprises a VH domain comprising the amino acid sequence of SEQ ID NO: 290 and a VL domain comprising the amino acid sequence of SEQ ID NO: 291, and the second cytokine receptor domain comprises a VH domain comprising the amino acid sequence of SEQ ID NO: 292 and a VL domain comprising the amino acid sequence of SEQ ID NO: 293; or b) the first cytokine receptor-binding domain comprises a VH domain comprising the amino acid sequence of SEQ ID NO: 292 and a VL domain comprising the amino acid sequence of SEQ ID NO: 293, and the second cytokine receptor domain comprises a VH domain comprising the amino acid sequence of SEQ ID NO: 290 and a VL domain comprising the amino acid sequence of SEQ ID NO: 291.

In one aspect, the Fc domains of the first and second antigen binding molecules comprising Fabs or scFv molecules as cytokine receptor-binding domains comprise a first Fc domain subunit and a second Fc domain subunit. In one aspect, the Fc domains of the first and second antigen binding molecules are IgG, particularly an IgG₁, Fc domains. In one aspect, the Fc domains of the first and second antigen binding molecules are human Fc domains. In one aspect, the Fc domains of the first and second antigen binding molecules comprise a modification promoting the association of the first and the second subunit of the Fc domains. In one aspect, the Fc domains of the first and second antigen binding molecules comprise one or more amino acid substitution that reduces binding to an Fc receptor and/or effector function. In one aspect, the cytokine receptor-binding domains are fused via peptide linkers to their respective fusion points. In one aspect, the peptide linkers comprise an amino acid sequence of SEQ ID NO: 280 or SEQ ID NO: 281. In one aspect, a) the first antigen binding molecule comprises a first polypeptide comprising an amino acid sequence of SEQ ID NO: 267, a second polypeptide comprising an amino acid sequence of SEQ ID NO: 28, a third polypeptide comprising an amino acid sequence of SEQ ID NO: 265 and a fourth polypeptide comprising an amino acid sequence of SEQ ID NO: 266, and the second antigen binding molecule comprises a first polypeptide comprising an amino acid sequence of SEQ ID NO: 272, a second polypeptide comprising an amino acid sequence of SEQ ID NO: 28, a third polypeptide comprising an amino acid sequence of SEQ ID NO: 270 and a fourth polypeptide comprising an amino acid sequence of SEQ ID NO: 271; or b) the first antigen binding molecule comprises a first polypeptide comprising an amino acid sequence of SEQ ID NO: 267, a second polypeptide comprising an amino acid sequence of SEQ ID NO: 28, a third polypeptide comprising an amino acid sequence of SEQ ID NO: 265 and a fourth polypeptide comprising an amino acid sequence of SEQ ID NO: 266, and the second antigen binding molecule comprises a first polypeptide comprising an amino acid sequence of SEQ ID NO: 274, a second polypeptide comprising an amino acid sequence of SEQ ID NO: 28, a third polypeptide comprising an amino acid sequence of SEQ ID NO: 270 and a fourth polypeptide comprising an amino acid sequence of SEQ ID NO: 273; or c) the first antigen binding molecule comprises a first polypeptide comprising an amino acid sequence of SEQ ID NO: 269, a second polypeptide comprising an amino acid sequence of SEQ ID NO: 28, a third polypeptide comprising an amino acid sequence of SEQ ID NO: 265 and a fourth polypeptide comprising an amino acid sequence of SEQ ID NO: 268, and the second antigen binding molecule comprises a first polypeptide comprising an amino acid sequence of SEQ ID NO: 268, a second polypeptide comprising an amino acid sequence of SEQ ID NO: 28, a third polypeptide comprising an amino acid sequence of SEQ ID NO: 270 and a fourth polypeptide comprising an amino acid sequence of SEQ ID NO: 271; or d) the first antigen binding molecule comprises a first polypeptide comprising an amino acid sequence of SEQ ID NO: 269, a second polypeptide comprising an amino acid sequence of SEQ ID NO: 28, a third polypeptide comprising an amino acid sequence of SEQ ID NO: 265 and a fourth polypeptide comprising an amino acid sequence of SEQ ID NO: 268, and the second antigen binding molecule comprises a first polypeptide comprising an amino acid sequence of SEQ ID NO: 274, a second polypeptide comprising an amino acid sequence of SEQ ID NO: 28, a third polypeptide comprising an amino acid sequence of SEQ ID NO: 270 and a fourth polypeptide comprising an amino acid sequence of SEQ ID NO: 273. In one aspect, the first antigen binding molecule comprises a first polypeptide comprising an amino acid sequence of SEQ ID NO: 276, a second polypeptide comprising an amino acid sequence of SEQ ID NO: 28 and a third polypeptide comprising an amino acid sequence of SEQ ID NO: 275, and the second antigen binding molecule comprises a first polypeptide comprising an amino acid sequence of SEQ ID NO: 277, a second polypeptide comprising an amino acid sequence of SEQ ID NO: 28 and a third polypeptide comprising an amino acid sequence of SEQ ID NO: 278.

### BRIEF DESCRIPTION OF THE FIGS.

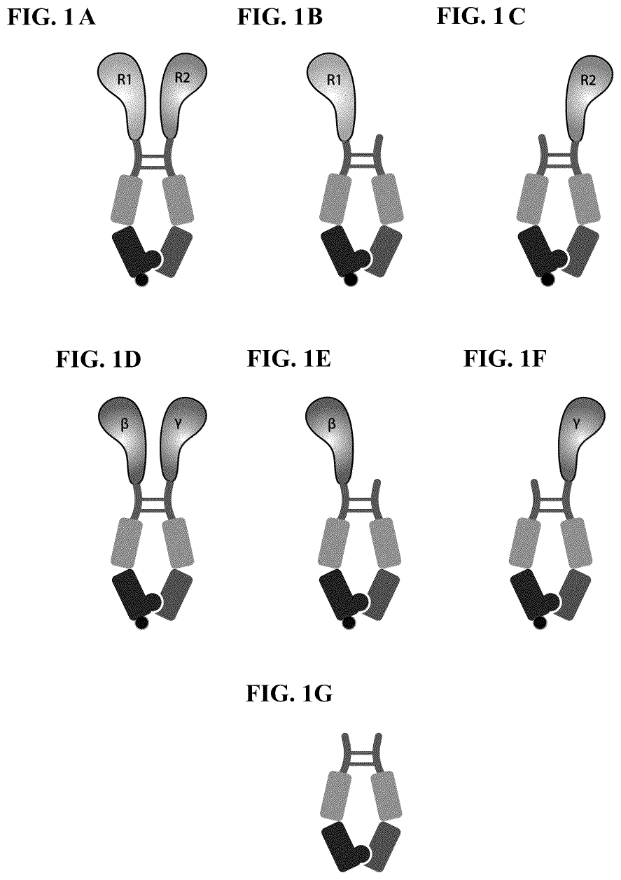

FIG. 1A-FIG. 1G. Schematic representations of antigens used in llama immunization, phage display and screening for the isolation of human IFNγR1, IFNγR2, IL-2Rβ and IL-2Rγ-specific single domain antibodies. For llama immunization a heterodimer formed of the extracellular domain (ECD) of human IFNγR1 fused to biotinylated Fc knob and human IFNγR2 ECD fused to Fc hole (FIG. 1A) and a heterodimer formed of the ECD of human IL-2Rβ fused to biotinylated Fc knob and human IL-2Rγ ECD fused to Fc hole (FIG. 1D) were generated. For phage display monovalent human IFNγR1 fused to biotinylated Fc (FIG. 1B), monovalent human IFNγR2 fused to biotinylated Fc (FIG. 1C), monovalent human IL-2Rβ fused to biotinylated Fc (FIG. 1E), monovalent human IL-2Rγ fused to biotinylated Fc (FIG. 1F) and soluble Fc (FIG. 1G) were generated.

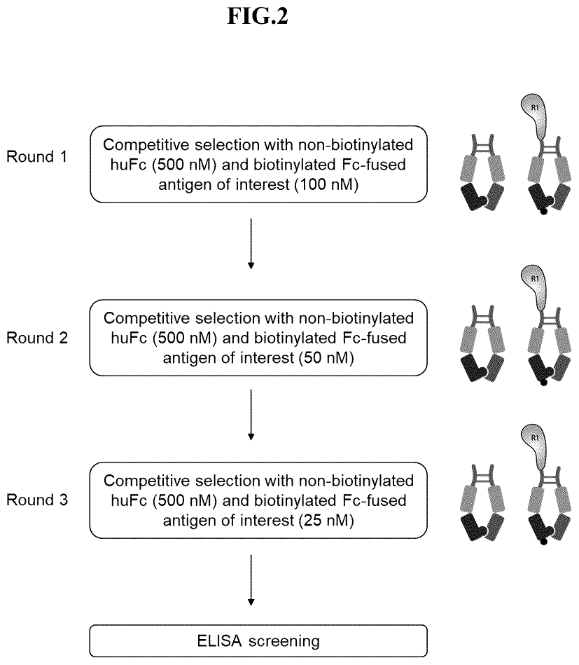

FIG. 2. Workflow for the enrichment of single domain antibodies with binding specificity for human cytokine receptor subunits by phage display.

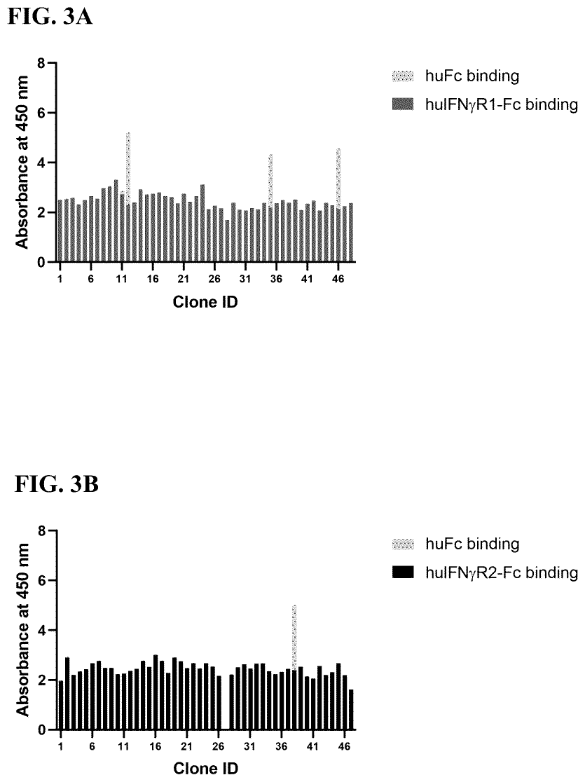

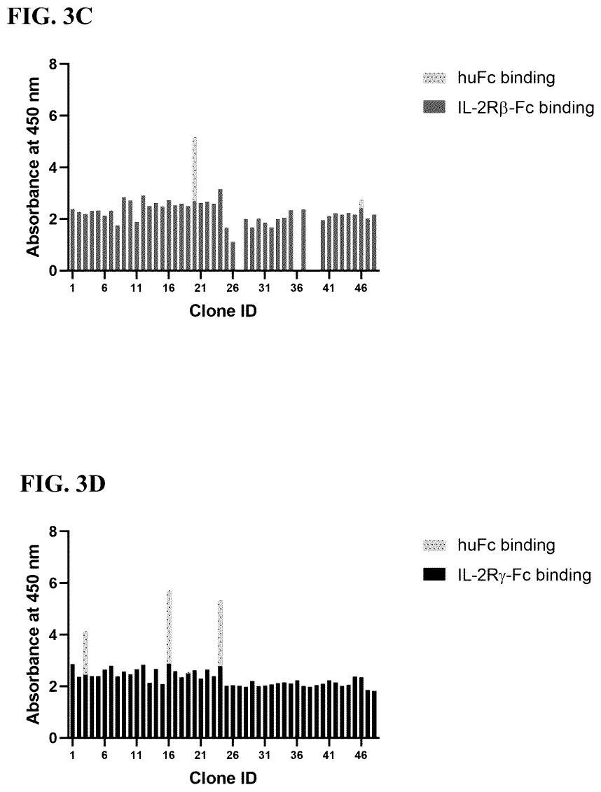

FIG. 3A-FIG. 3.D. Specificity screening of single domain antibodies by ELISA. A random set of soluble VHH domains selected for human IFNγR1 specificity following three rounds of phage display were tested for binding to immobilized human IFNγR1-Fc and immobilized human Fc (FIG. 3A). VHH domains with human IFNγR2 specificity following three rounds of phage display were also tested for antigen specificity by ELISA (FIG. 3B). VHH domains selected for human IL-2Rβ specificity following three rounds of phage display were tested for binding to immobilized human IL-2Rβ-Fc and immobilized human Fc (FIG. 3C). VHH domains with human IL-2Rγ specificity following three rounds of phage display were also tested for antigen specificity by ELISA (FIG. 3D). Absorbance at 450 nm indicating the binding response to each target is represented as a stacked bar chart.

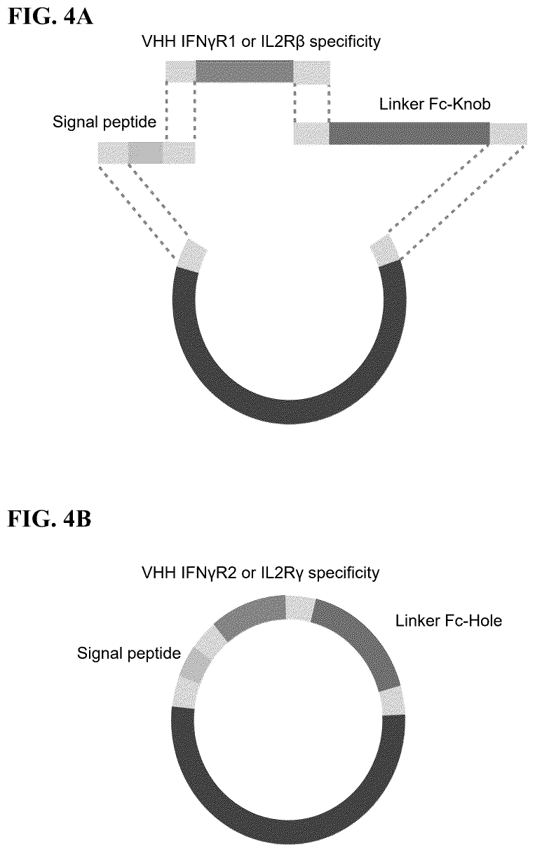

FIG. 4A-FIG. 4B. Format conversion of single domain antibody fragments. Round 3 VHH library variants with IFNγR1 or IL-2Rβ specificity were fused to Fc-Knob and cloned into a mammalian cell expression vector using Gibson cloning method (FIG. 4A). Round 3 VHH library variants with IFNγR2 or IL-2Rγ specificity were fused to Fc-hole using the same method (FIG. 4B).

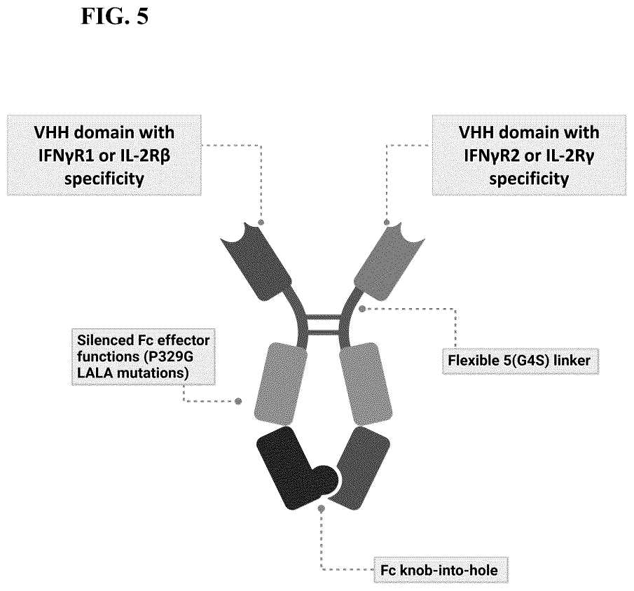

FIG. 5. Bispecific heavy chain antibody comprising IFNγR1-IFNγR2 or IL-2Rβ-IL-2Rγ VHH domain pairs. VHH domains with IFNγR1 or IL-2Rβ specificity were fused to Fc-knob and VHH domains targeting IFNγR2 or IL-2Rγ were fused to Fc-hole. Flexible 5(G4S) linkers were used to fuse VHH moieties to Fc chains. Bispecific heavy chain antibodies were generated via knob-into-hole assembly of Fc chains. Effector functions of the Fc were silenced via incorporation of P329G LALA mutations in the CH2 domain.

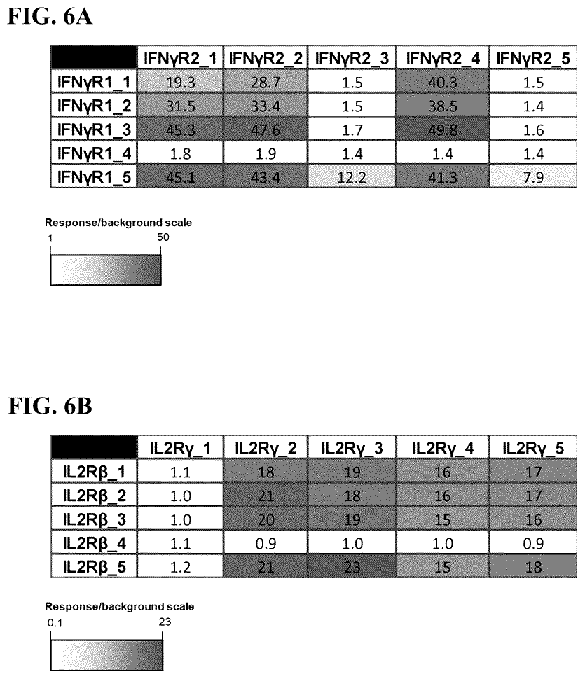

FIG. 6A-FIG. 6B. Functional screening of bispecific heavy chain antibodies comprising IFNγR1-IFNγR2 or IL-2Rβ-IL-2Rγ VHH domain pairs. HEK-Blue IFNγ cells were incubated with IFNγ agonistic bispecific heavy chain antibodies comprising IFNγR1 and IFNγR2 VHH domain pairs generated using a 5×5 bispecificity matrix (FIG. 6A). HEK-Blue IL-2 reporter cells were incubated with IL-2 agonistic bispecific heavy chain antibodies comprising IL-2Rβ and IL-2Rγ VHH pairs generated using a 5×5 bispecificity matrix (FIG. 6B). IFNγR activity and IL-2R activity was quantified by absorbance at 650 nm and fold response/background is shown for each treatment.

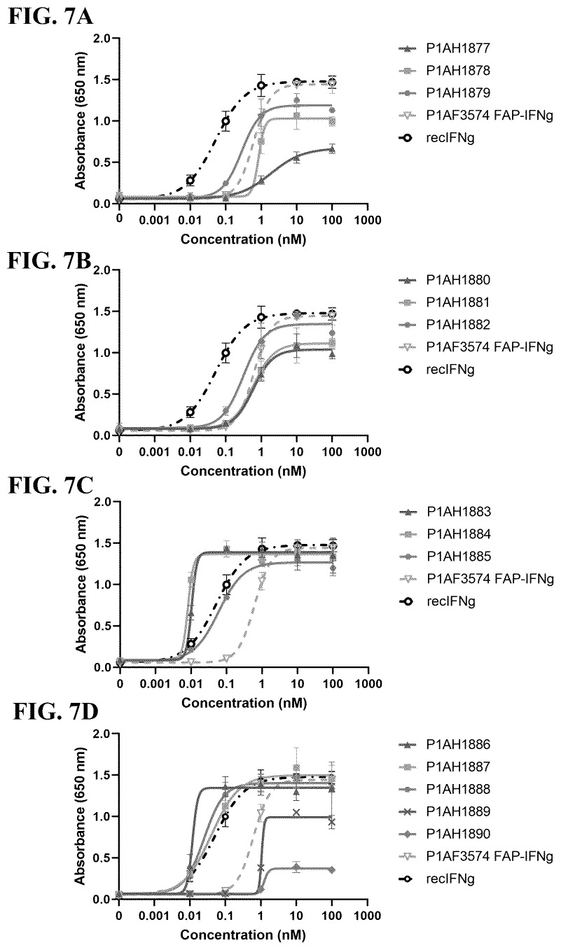

FIG. 7A-FIG. 7D. Characterisation of dose-dependent response for IFNγ agonistic heavy chain antibodies. Previously identified IFNγR agonists (FIG. 6A, Table 1) were screened for dose-dependent IFNγR activity in HEK-Blue IFNγ cells, characterized by absorbance at 650 nm. The responses of different IFNγ agonistic heavy chain antibodies, are grouped according to the anti-IFNγR1 VHH clone, i.e. within each graph the anti-IFNγR2 VHH clone is variable while the anti-IFNγR1 VHH clone remains constant: IFNγR1_1 clone (FIG. 7A), IFNγR1_2 clone (FIG. 7B), IFNγR1_3 clone (FIG. 7C) and IFNγR1_5 clone (FIG. 7D). Responses were compared to recombinant human IFNγ (black dashed line) and FAP-IFNγ (P1AF3574; light grey dashed line).

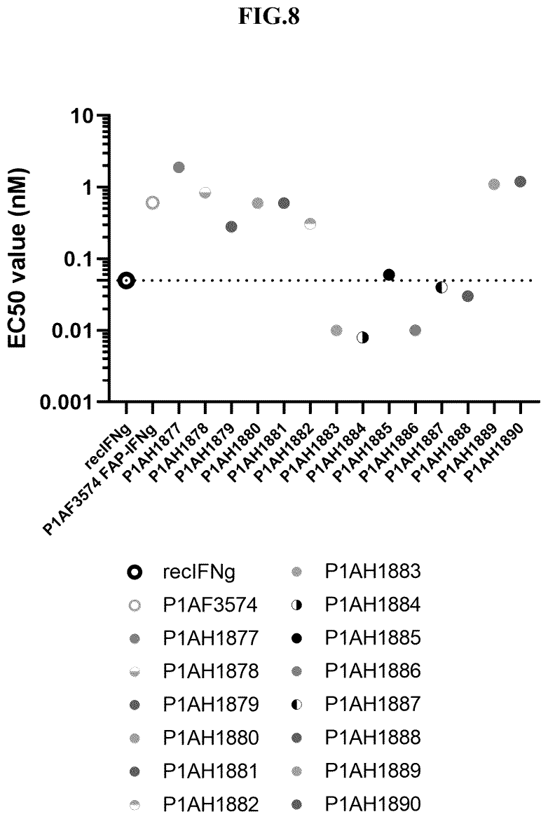

FIG. 8. Comparison of EC50 values for IFNγ agonistic heavy chain antibodies. EC50 values were derived from HEK-Blue assays (FIG. 7) using GraphPad Prism software. Values are depicted in nanomolar concentration with recombinant human IFNγ highlighted as a reference (dotted line).

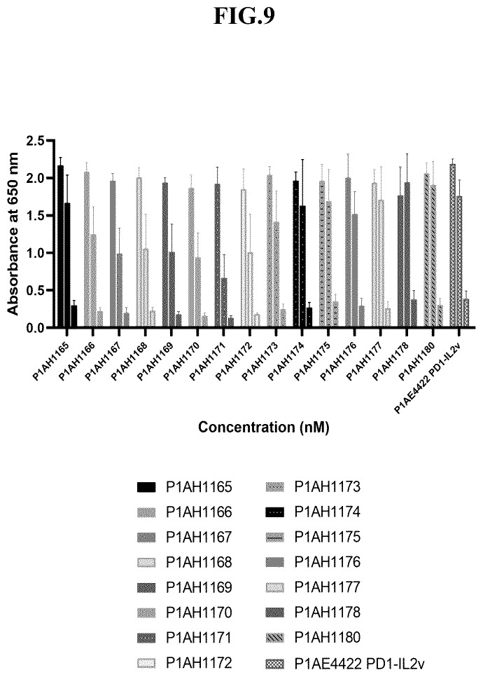

FIG. 9. Characterization of dose-dependent response for IL-2R agonistic heavy chain antibodies. Previously identified IL-2R agonists (FIG. 6B, Table 2) were screened for dose-dependent IL-2R agonism in HEK-Blue IL-2 cells. Anti-PD1-IL2v (P1AE4422) was used as a reference molecule and each molecule was incubated with reporter cells at the following concentrations: 20 nM, 0.8 nM, 0.032 nM shown from left to right. IL-2R activity was quantified by absorbance at 650 nm; mean values from technical triplicates are shown with error bars representing standard deviation.

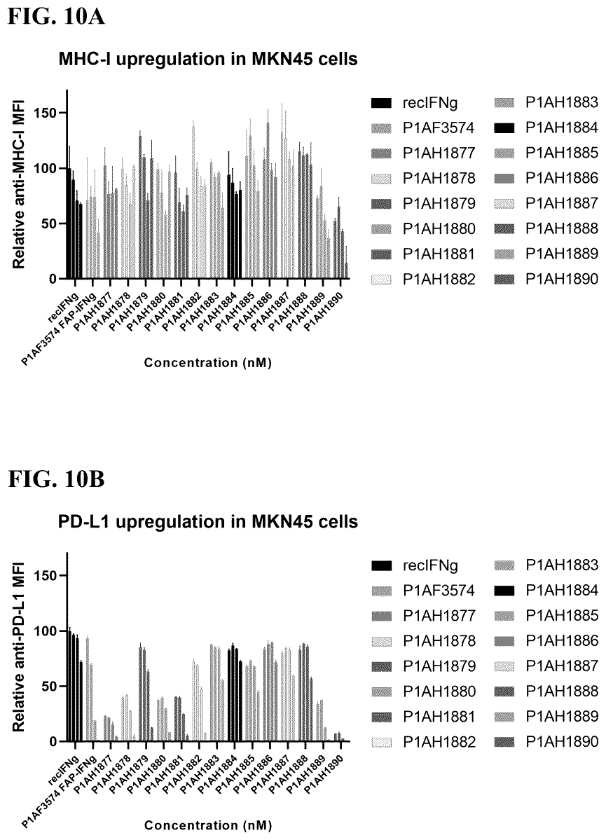

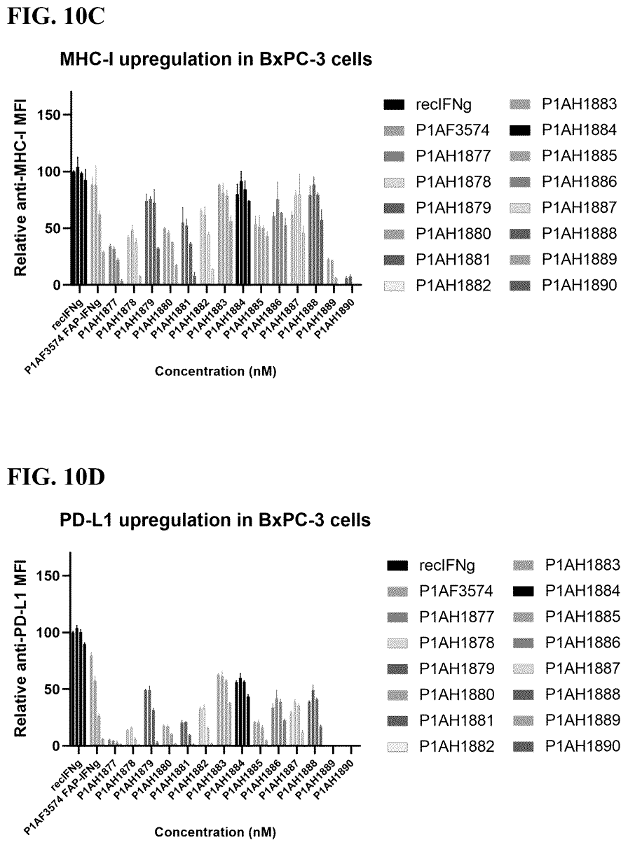

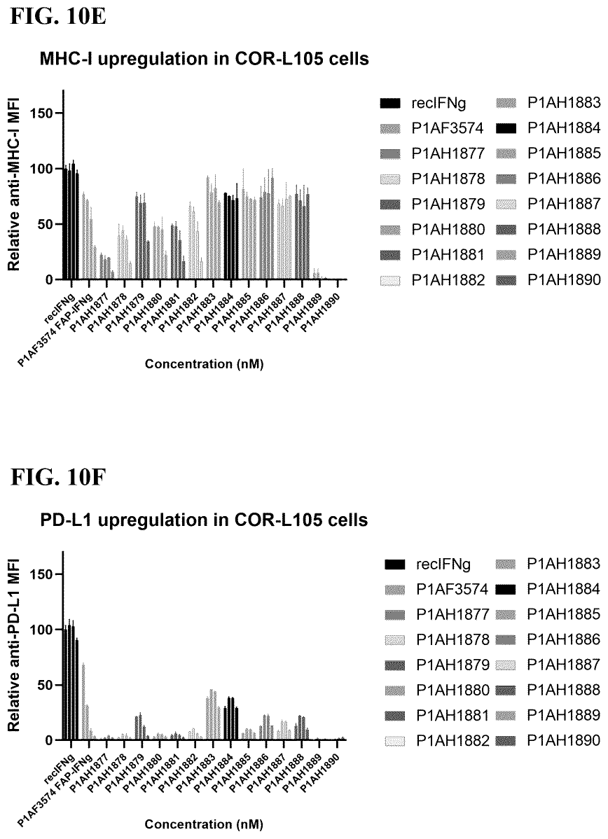

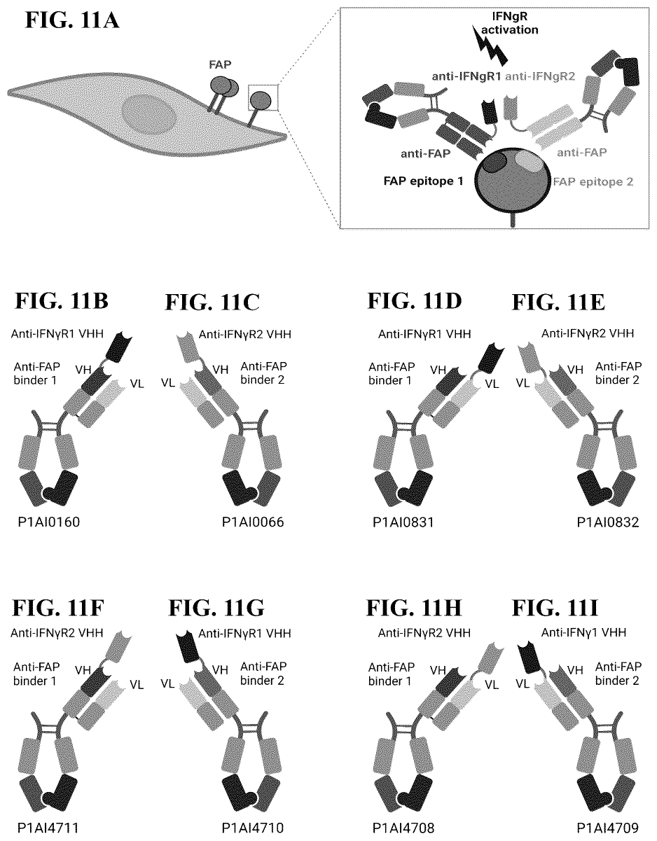

FIG. 10A-FIG. 10F. MHC-I and PD-L1 expression on tumor cells following 72 h treatment of IFNγ agonists. Previously identified IFNγR agonists (P1AH1877-P1AH1890) and reference molecules (recombinant human IFNγ and FAP-IFNγ) were incubated with tumor cells at the following concentration of treatment: 100, 10, 1 and 0.1 nM shown from left to right for each treatment. Measurements were blank-subtracted and normalized to recombinant human IFNγ response at the highest concentration (100 nM). Responses are depicted for MHC-I expression on MKN45 cells (FIG. 10A), PD-L1 expression on MKN45 cells (FIG. 10B), for MHC-I expression on Bxpc3 cells (FIG. 10C), PD-L1 expression on Bxpc3 cells (FIG. 10D), MHC-I expression on CorL105 cells (FIG. 10E) and PD-L1 expression on CorL105 cells (FIG. 10F).

FIG. 11A-FIG. 11I. Concept of split dual-targeted IFNγR agonists. FAP-dependent biparatopic assembly of split IFNγ mimetics (FIG. 11A). Note that for illustrative simplification, only one monomer of the FAP dimer is represented in the inset. VHH domains with IFNγR1 and IFNγR2 specificity were fused to the N-terminus of two different anti-FAP binding domains, via VH or VL fusion points, resulting in eight configurations within the permutation space: IFNγR1-VHH fused to the VH of FAP binder 1 (FIG. 11B), IFNγR2-VHH fused to the VH of FAP binder 2 (FIG. 11C), IFNγR1-VHH fused to the VL of FAP binder 1 (FIG. 11D), IFNγR2-VHH fused to the VL of FAP binder 2 (FIG. 11E), IFNγR2-VHH fused to the VH of FAP binder 1 (FIG. 11F), IFNγR1-VHH fused to the VH of FAP binder 2 (FIG. 11G), IFNγR2-VHH fused to the VL of FAP binder 1 (FIG. 11H) and IFNγR1-VHH fused to the VL of FAP binder 2 (FIG. 11I). All molecules have the same Fc characteristics as described in FIG. 5.

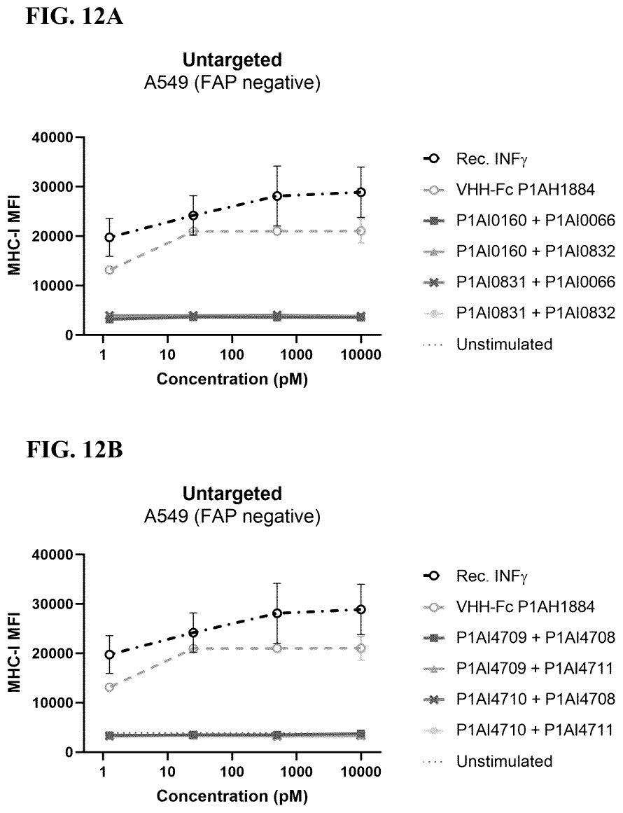

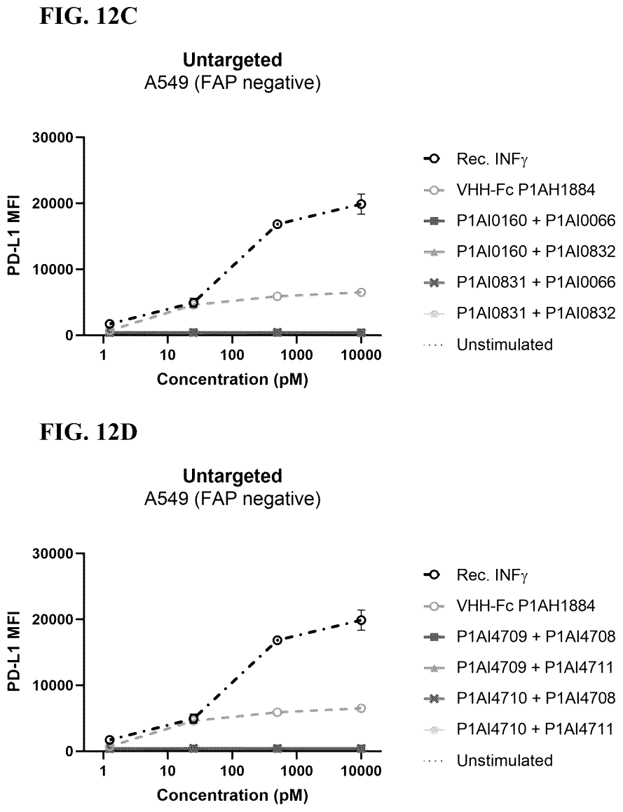

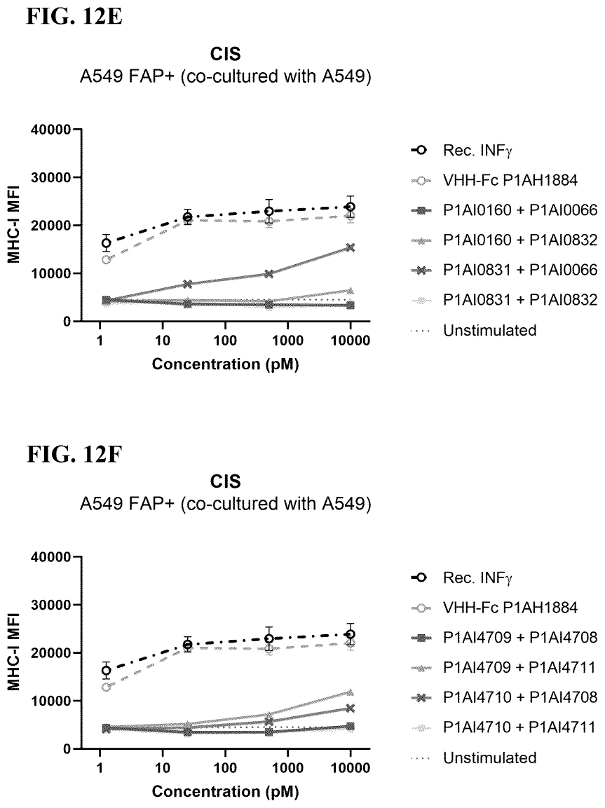

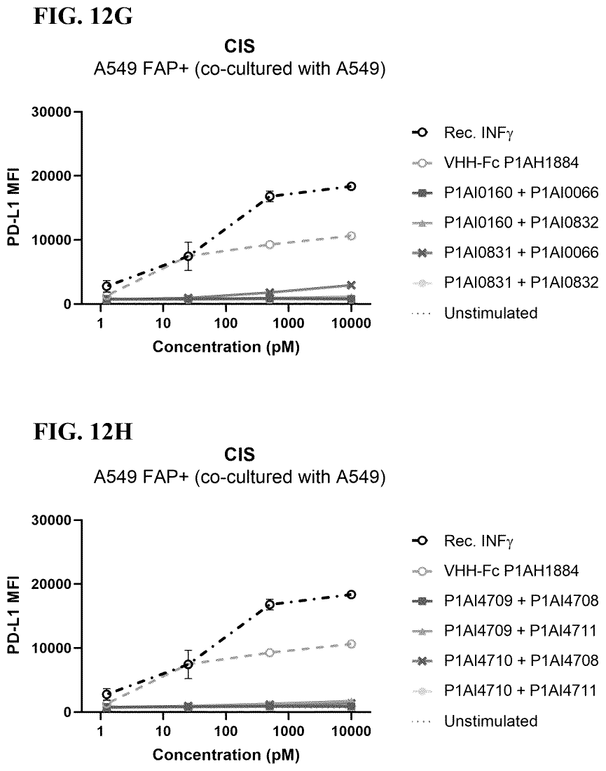

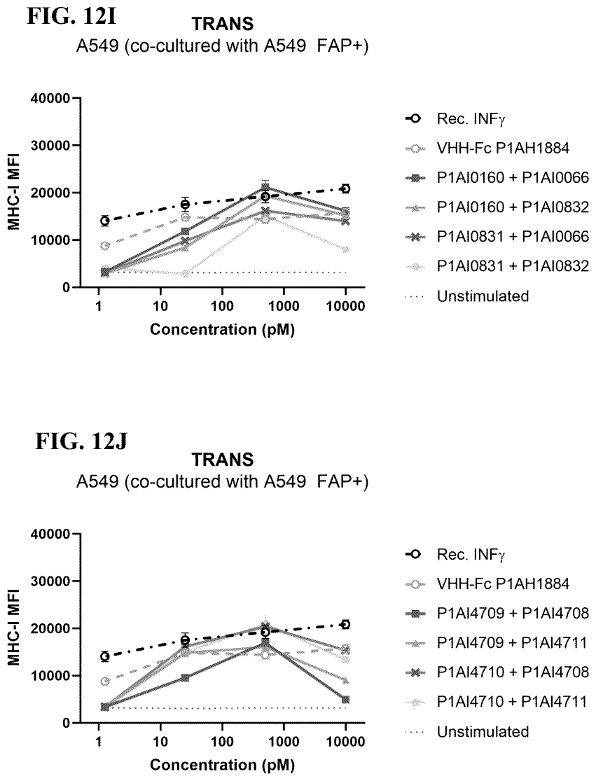

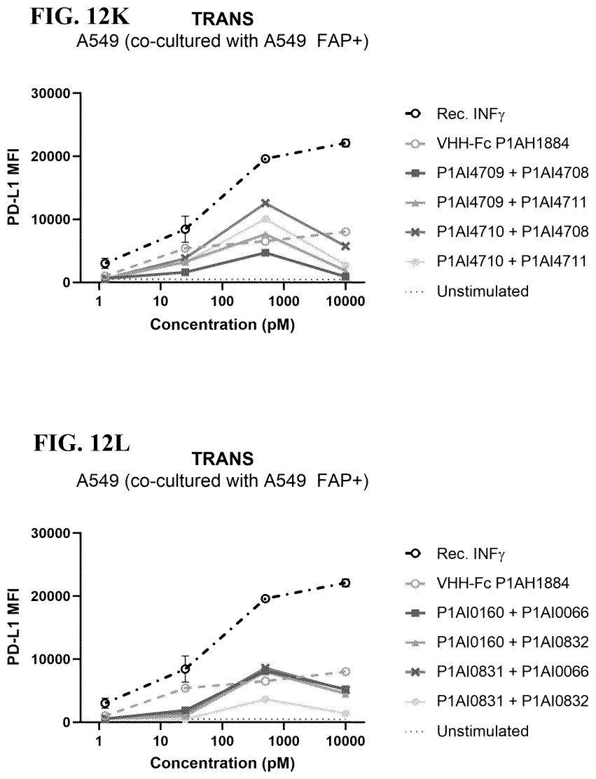

FIG. 12A-FIG. 12L. Assessment of IFNγR activity mediated by FAP-dependent split IFNγR agonists. The eight split IFNγ mimetics (FIG. 11B-I) were paired in biparatopic assembly combinations and tested for IFNγR agonism characterized by MHC-I and PD-L1 upregulation. FAP-negative A549 cells were tested for untargeted, non-specific MHC-I (FIG. 12A, FIG. 12B) and PD-L1 upregulation (FIG. 12C, FIG. 12D) by split IFNγ mimetics. A549 FAP-positive cells were co-cultured with differentially labeled A549 FAP-negative cells to assess FAP-dependent activity of IFNγ mimetics in cis and trans: MHC-I upregulation in cis (FIG. 12E and FIG. 12F), PD-L1 upregulation in cis (FIG. 12G and FIG. 12H), MHC-I upregulation in trans (FIG. 12I and FIG. 12J) and PD-L1 upregulation in trans (FIG. 12K and FIG. 12L). Recombinant IFNγ and the IFNγ agonistic heavy chain antibody P1AH1884 (as depicted in FIG. 5) were used as a reference. Median fluorescent intensity (MFI) of MHC-I and PD-L1 expression levels were analyzed in FlowJo; mean values from technical duplicates are shown with error bars representing standard deviation.

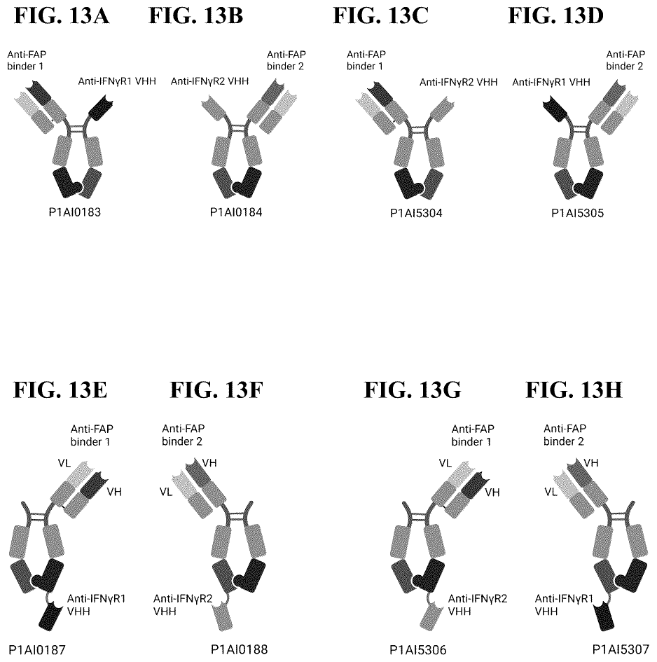

FIG. 13A-FIG. 13H. Additional formats of split dual-targeted IFNγR agonists. VHH domains with IFNγR1 and IFNγR2 specificity were distanced from the anti-FAP binders via fusion to the N-terminus of the opposite Fc chain, resulting in four configurations: IFNγR1 and FAP binder 1 (FIG. 13A), IFNγR2 and FAP binder 2 (FIG. 13B), IFNγR2 and FAP binder 1 (FIG. 13C) and IFNγR1 and FAP binder 2 (FIG. 13D). Alternatively, the VHH domains with IFNγR1 and IFNγR2 specificity were distanced from the anti-FAP binders via fusion to the C-terminus of the same Fc chain, resulting in four configurations: IFNγR1 and FAP binder 1 (FIG. 13E), IFNγR2 and FAP binder 2 (FIG. 13F), IFNγR2 and FAP binder 1 (FIG. 13G) and IFNγR1 and FAP binder 2 (FIG. 13H). The anti-FAP binders in FIG. 13E-H comprise CrossFab VH/VL engineering (binder 1) and CH1-CL charges (binder 2) to subsequently allow for additional pairings. All molecules have the same Fc characteristics as described in FIG. 5.

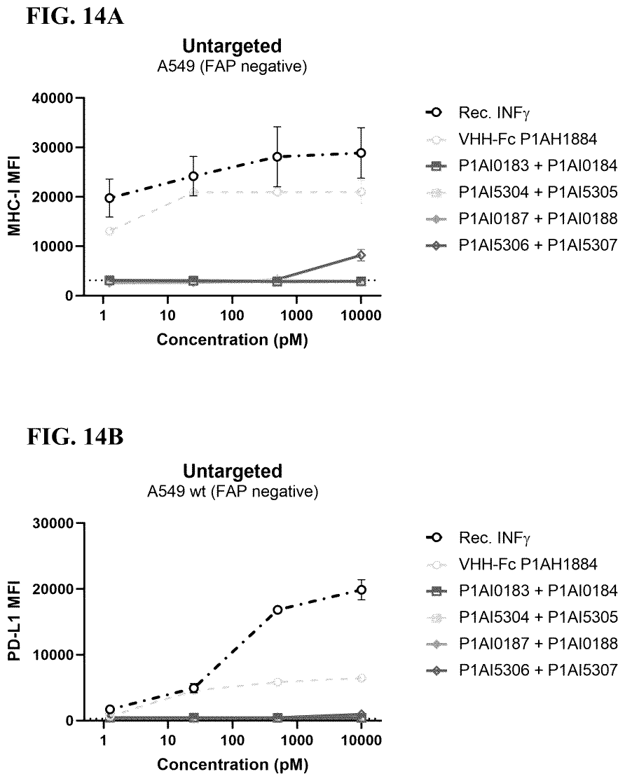

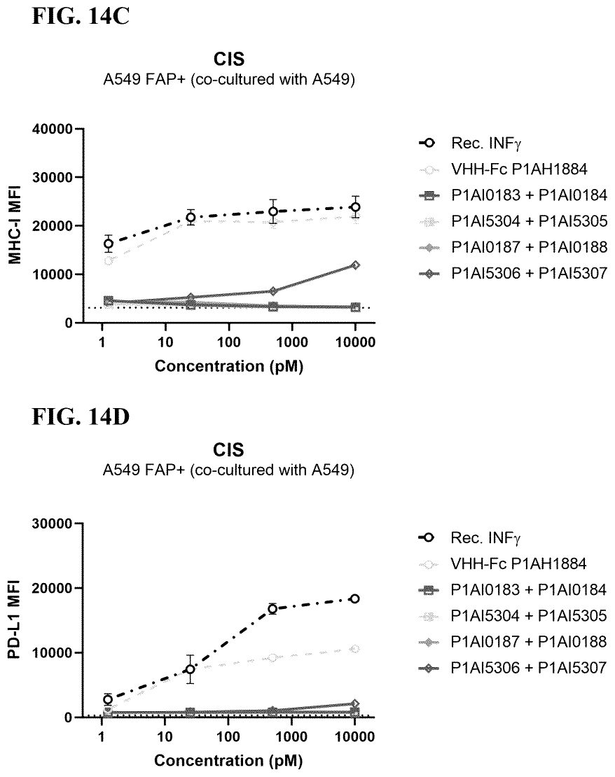

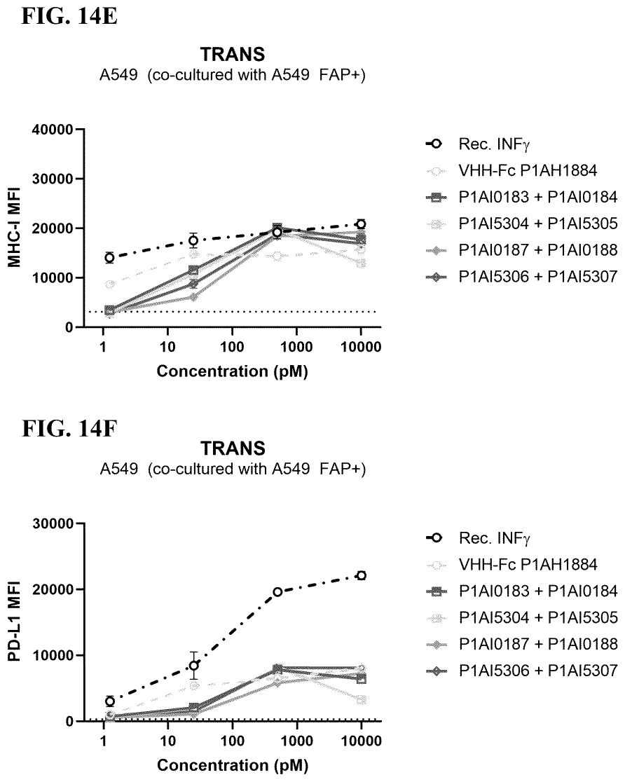

FIG. 14A-FIG. 14F. Assessment of IFNγR activity mediated by additional FAP-dependent split IFNγR agonists. Four combinations of molecules depicted in FIG. 13 and reference molecules were tested for IFNγR agonism characterized by MHC-I and PD-L1 upregulation. A549 cells lacking FAP expression were tested for FAP-independent MHC-I upregulation (FIG. 14A) and PD-L1 upregulation (FIG. 14B) of split IFNγ mimetics. A549 FAP-positive cells were co-cultured with differentially labeled A549 FAP-negative cells to assess FAP-dependent activity of IFNγ mimetics by MHC-I upregulation in cis (FIG. 14C), PD-L1 upregulation in cis (FIG. 14D), MHC-I upregulation in trans (FIG. 14E) and PD-L1 upregulation in trans (FIG. 14F). Recombinant IFNγ and an IFNγ agonistic heavy chain antibody (P1AH1884) were used as reference molecules. Median fluorescent intensity (MFI) of MHC-I and PD-L1 expression levels were analyzed in FlowJo; mean values from technical duplicates are shown with error bars representing standard deviation.

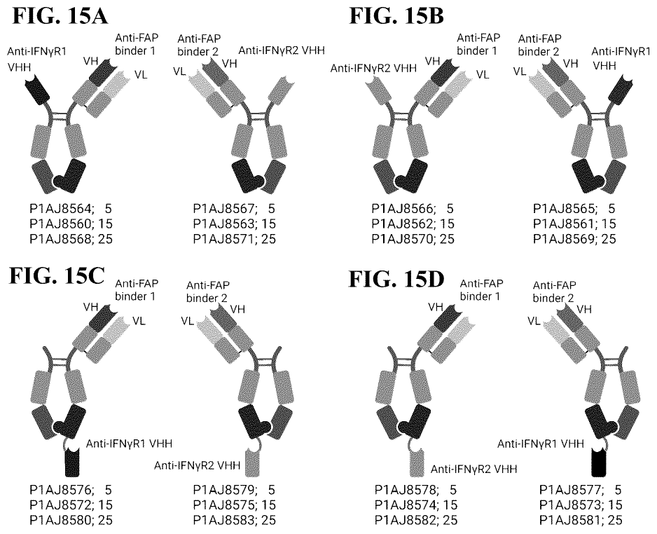

FIG. 15A-FIG. 15D. Concept of split FAP-targeted IFNγ agonists with different linker lengths. A single VHH domain with specificity for either IFNγR1 or IFNγR2 was engineered to be linked to the hinge region at the N-terminus (top row) or to the C-terminus (bottom row) of the Fc region within an antibody construct containing a FAP-binding Fab domain. The constructs utilized one of two different FAP-binding Fab domains that do not compete for binding. Flexible linkers, consisting of 5, 15, or 25 amino acids, denoted by the number following a semicolon after the molecule ID, were used to connect the Fc region to the VHH domain. This resulted in a series of 24 unique configurations which can be paired to produce 12 functional FAP-targeted IFNγ agonists: P1AJ8564+P1AJ8567, P1AJ8560+P1AJ8563 and P1AJ8568+P1AJ8571 (FIG. 15A); P1AJ8566+P1AJ8565, P1AJ8562+P1AJ8561 and P1AJ8570+P1AJ8569 (FIG. 15B); P1AJ8576+P1AJ8579, P1AI0187+P1AJ8575 and P1AJ8580+P1AJ8583 (FIG. 15C); P1AJ8578+P1AJ8577, P1AJ8574+P1AJ8573 and P1AJ8582+P1AJ8581 (FIG. 15D).

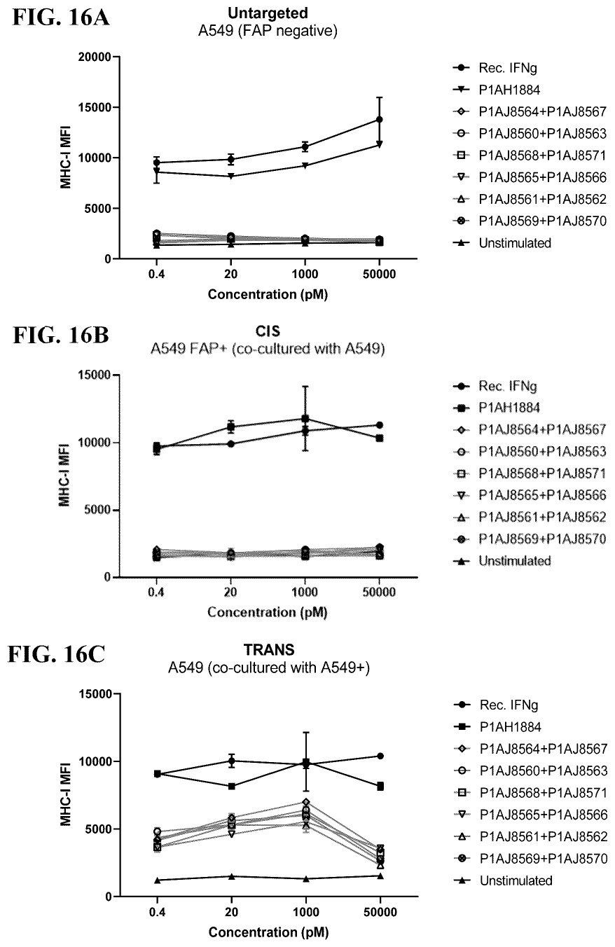

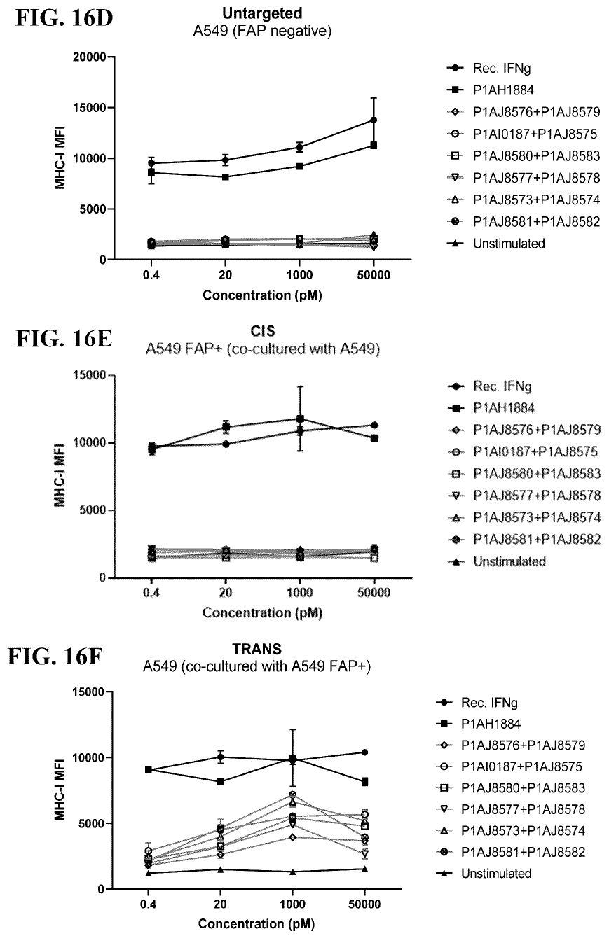

FIG. 16A-FIG. 16F. Assessment of IFNγR activity mediated by additional FAP-dependent split IFNγR agonists. Twelve combinations of molecules depicted in FIG. 15 and reference molecules were tested for IFNγR agonism characterized by MHC-I upregulation. Test compounds were incubated with A549 FAP-negative cells alone (untargeted condition; FIG. 16A and FIG. 16D) or in combination with A549 FAP-positive cells, wherein cis signaling (FAP binding on A549 FAP-positive cells and activity on A549 FAP-positive cells; FIG. 16B and FIG. 16E) and trans signaling (FAP binding on A549 FAP-positive cells and activity on A549 FAP-negative cells, FIG. 16C and FIG. 16F) was assessed. Recombinant IFNγ and an IFNγ agonistic heavy chain antibody (P1AH1884) were used as reference molecules. Median fluorescent intensity (MFI) of MHC-I expression levels were analyzed in FlowJo; mean values from technical duplicates are shown with error bars representing standard deviation.

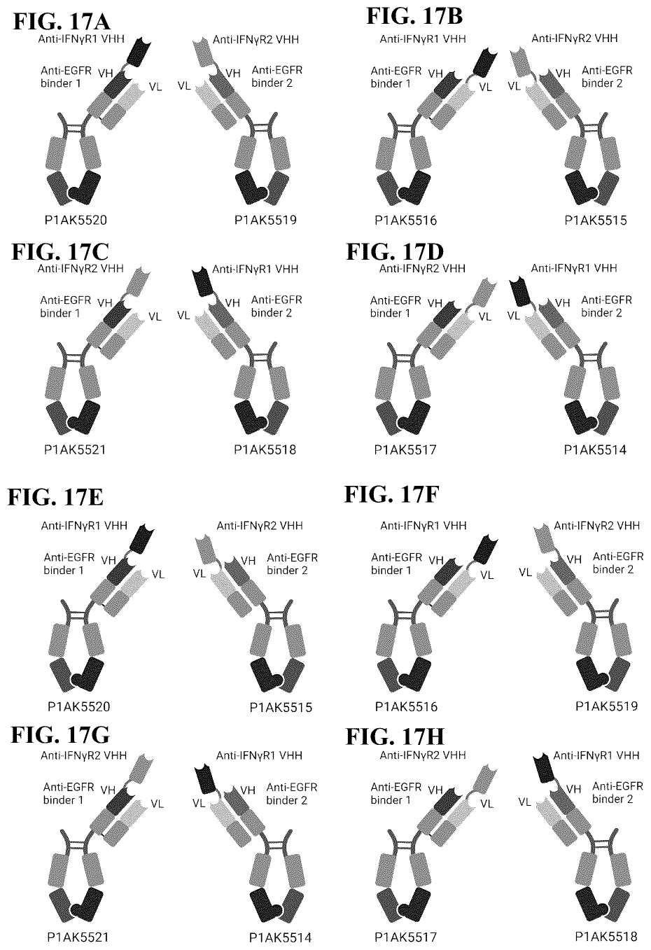

FIG. 17A-FIG. 17H. Concept of split EGFR-targeted IFNγ agonists. VHH domains with IFNγR1 and IFNγR2 specificity were fused to the N-terminus of two different anti-EGFR binding domains, via VH or VL fusion points, resulting in eight individual format configurations (P1AK5514−P1AK5521) that can be paired to produce the eight functional assemblies depicted: P1AK5520+P1AK5519 (FIG. 17A), P1AK5516+P1AK5515 (FIG. 17B), P1AK5521+P1AK5518 (FIG. 17C), P1AK5517+P1AK5514 (FIG. 17D), P1AK5520+P1AK5515 (FIG. 17E), P1AK5516+P1AK5519 (FIG. 17F), P1AK5521+P1AK5514 (FIG. 17G) and P1AK5517+P1AK5518 (FIG. 17H).

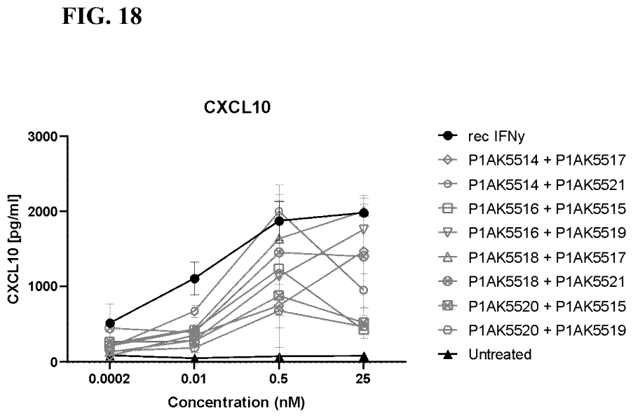

FIG. 18. Functional activity of EGFR-targeted IFNγ agonists. Test compounds were incubated with target cells together with PBMCs and CXCL10 production was assessed after 24 h stimulation. Recombinant IFNγ was used as reference. Shown are mean+/−SD of technical duplicates for each tested molecule concentration.

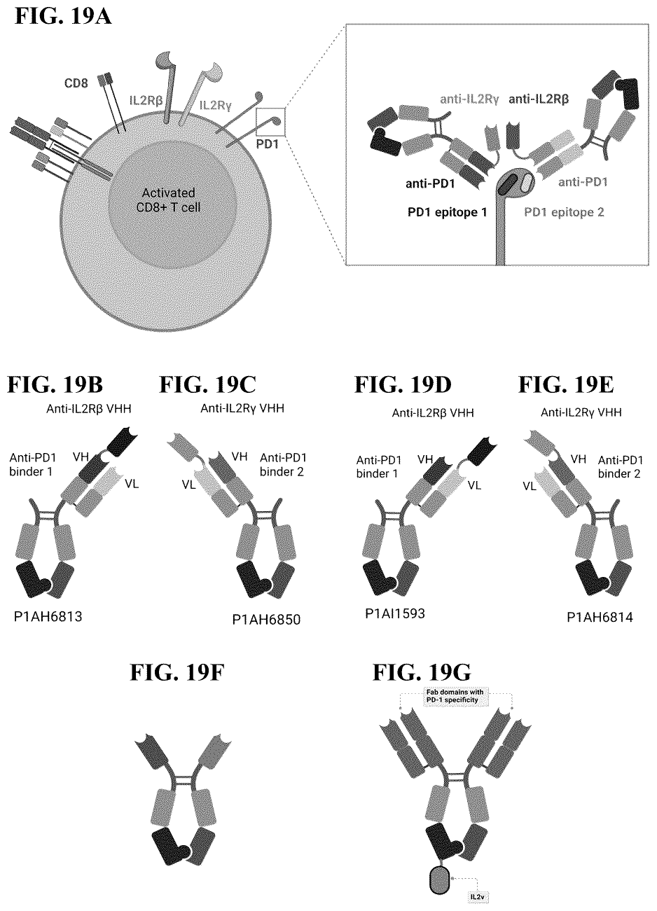

FIG. 19A-FIG. 19G. Concept of split dual-targeted IL-2 agonists. PD-1-dependent biparatopic assembly of split IL-2 mimetics (FIG. 19A). VHH domains with IL-2Rβ and IL-2Rγ specificity were fused to the N-terminus of two different anti-PD-1 binding domains, via VH or VL fusion points: IL-2Rβ-VHH fused to the VH of PD-1 binder 1 (FIG. 19B), IL-2Rγ-VHH fused to the VL of PD-1 binder 2 (FIG. 19C), IL-2Rβ-VHH fused to the VL of PD-1 binder 1 (FIG. 19D) and IL-2Rγ-VHH fused to the VH of PD-1 binder 2 (FIG. 19E). An IL-2 agonistic heavy chain antibody (FIG. 19F) and PD-1-IL2v (FIG. 19G) served as reference molecules. All molecules have the same Fc characteristics as described in FIG. 5.

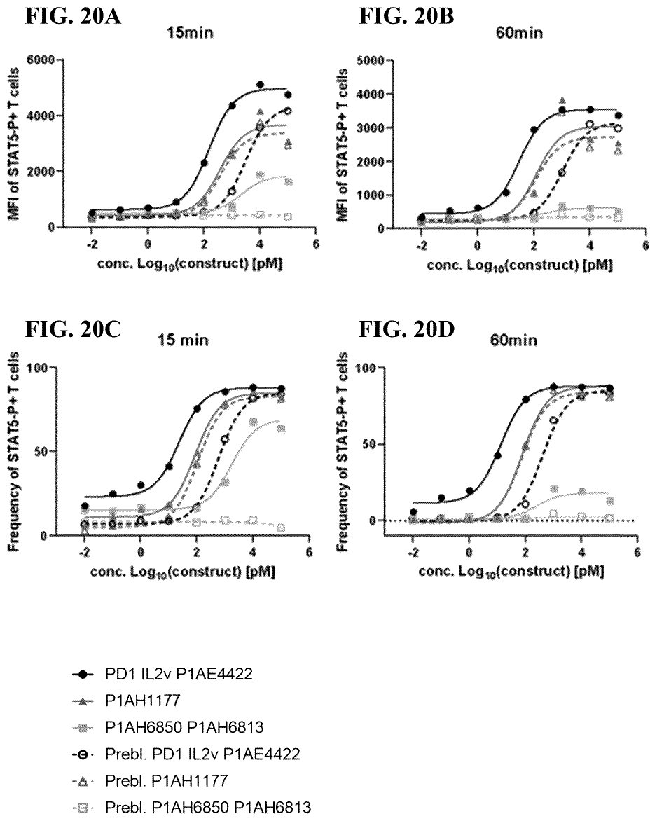

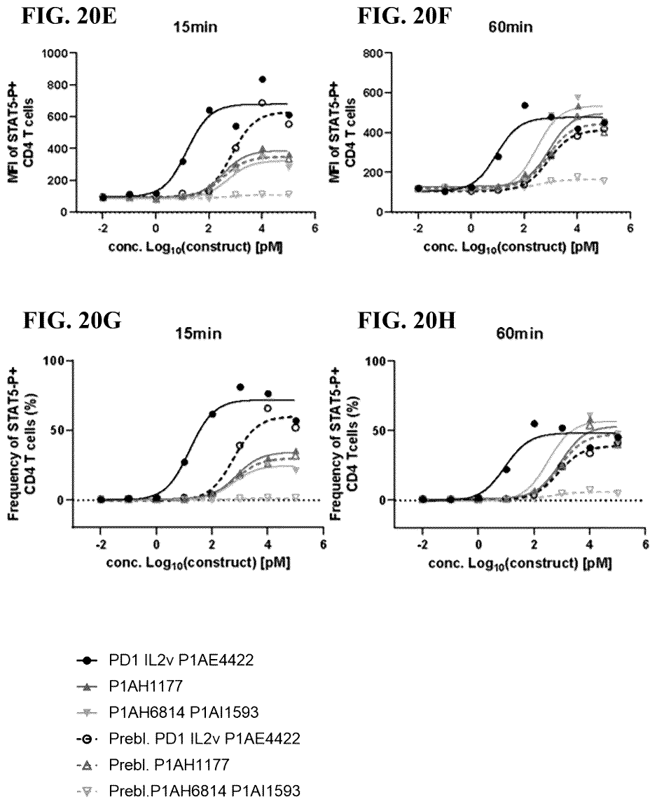

FIG. 20A-FIG. 20H. Phosphorylation of STAT5 on CD4 T cells after 15 min and 60 min incubation with IL-2R agonists. Activated T cells expressing PD-1 (PD-1⁺ subset) and activated T cells preblocked with anti-PD-1 antibodies (PD-1⁻ subset) were differentially labeled and treated with two combinations of split dual-targeted IL-2 mimetics: P1AH6850+P1AH6813 (FIG. 20A-FIG. 20D), P1AH6814+P1AI1593 (FIG. 20E-FIG. 20H). The IL-2 mimetic heavy chain antibody (P1AH1177) and PD1-IL2v (P1AE4422) were used as reference molecules. MFI and frequency of STAT5-P⁺ cells were measured by FACS and responses were shown for PD-1⁺ subset (solid line) and PD-1⁻ subset (dashed line).

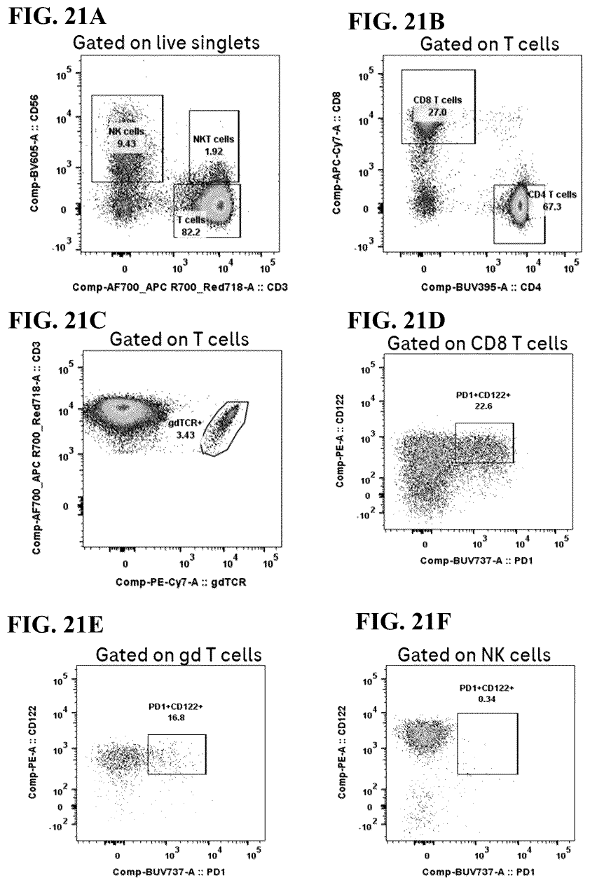

FIG. 21A-FIG. 21F. Flow cytometry gating strategy and baseline receptor expression. Isolated PBMCs from two healthy donors were stained with viability dye and fluorescent dye-conjugated antibodies against cell surface antigens. Gating strategy to identify five distinct cell types: NK cells and NKT cells (FIG. 21A); CD4 T cells and CD8 T cells (FIG. 21B); and γδ T cells (FIG. 21C). Representative plots showing PD-1 and IL-2Rβ co-expression on CD8 T cells (FIG. 21D), γδ T cells (FIG. 21E) and NK cells (FIG. 21F).

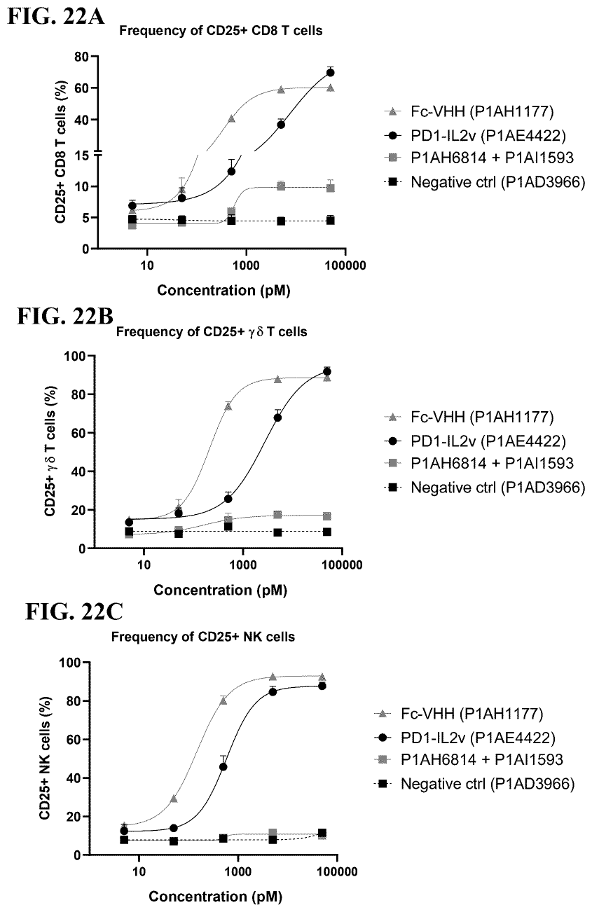

FIG. 22A-FIG. 22C. CD25 expression on PBMC subsets induced by PD-1 targeted IL-2R agonist. Frequency of CD25 expressing CD8 T cells (FIG. 22A), γδ T cells (FIG. 22B) and NK cells (FIG. 22C) were determined by flow cytometry after five days of test compounds co-incubation with freshly isolated CTV-labelled PBMCs. PD1-IL2v (P1AE4422) and Fc-VHH (P1AH1177) were used as reference. A non-binding DP47 antibody (P1AD3966) was used as negative control. Shown are mean values+/−SEM of two PBMC donors combined and technical duplicates for each tested molecule concentration.

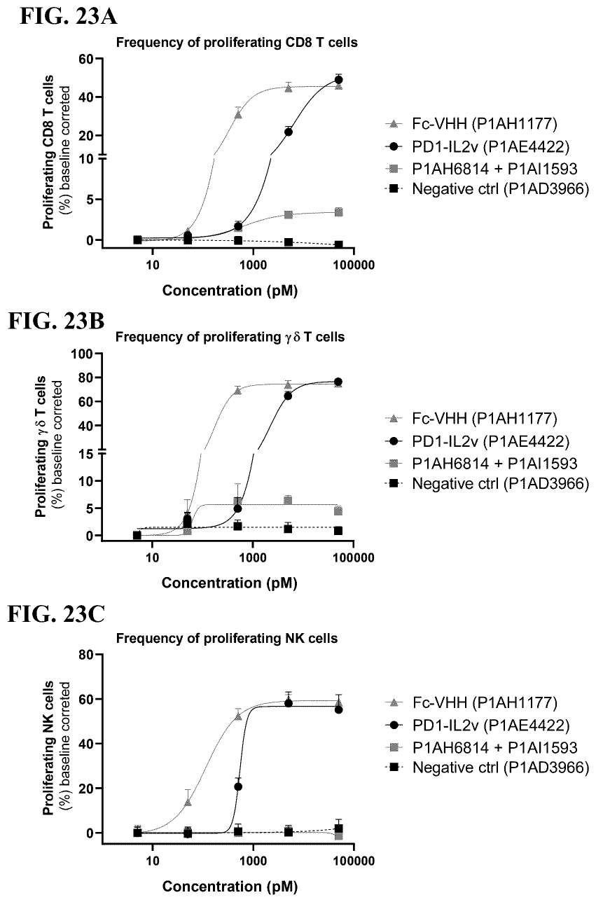

FIG. 23A-FIG. 23C. Proliferation of PBMC subsets induced by PD-1 targeted IL-2R agonist. Frequency of proliferating CD8 T cells (FIG. 23A), γδ T cells (FIG. 23B) and NK cells (FIG. 23C) were determined by flow cytometry after five days of test compounds co-incubation with freshly isolated CTV-labelled PBMCs. Proliferating cells were identified based on cell division visualized with CTV staining. PD1-IL2v (P1AE4422) and Fc-VHH (P1AH1177) were used as reference. A non-binding DP47 antibody (P1AD3966) was used as negative control. Shown are mean values+/−SEM of two PBMC donors combined and technical duplicates for each tested molecule concentration.

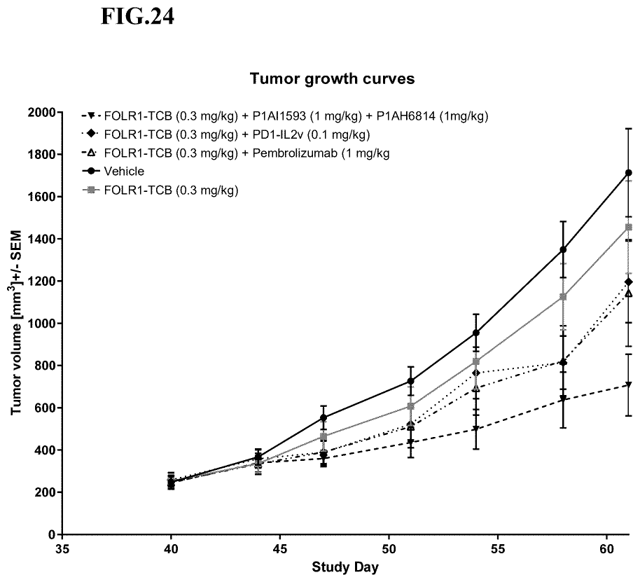

FIG. 24. Presents the results of an efficacy experiment evaluating PD-1 targeted IL-2R agonist (P1AI1593 and P1AH6814), PD1-IL2v immunoconjugate (P1AE4422) and Pembrolizumab in combination with FOLR1-TCB Mab. The BC004 human breast carcinoma PDX cells were injected intra mammary fat pad in humanized BRGS47 mice to study tumor growth inhibition in a breast orthotopic xenograft model. The amount of antibodies injected per mouse in mg/kg is the following: P1AI1593, P1AH6814 and Pembrolizumab 1 mg/kg, PD1-IL2v 0.1 mg/kg and FOLR1-TCB 0.3 mg/kg. The antibodies were injected i.v. once weekly for 4 weeks. The combination FOLR1-TCB 0.3 mg/kg+PD1-IL2 targeted IL-2R agonists (P1AI1593 day 1+P1AH6814 day 2) mediated superior efficacy in terms of tumor growth inhibition compared to FOLR1-TCB single agent treatment and the other combination groups: FOLR1-TCB 0.3 mg/kg+PD1-IL2v and FOLR1-TCB 0.3 mg/kg+Pembrolizumab.

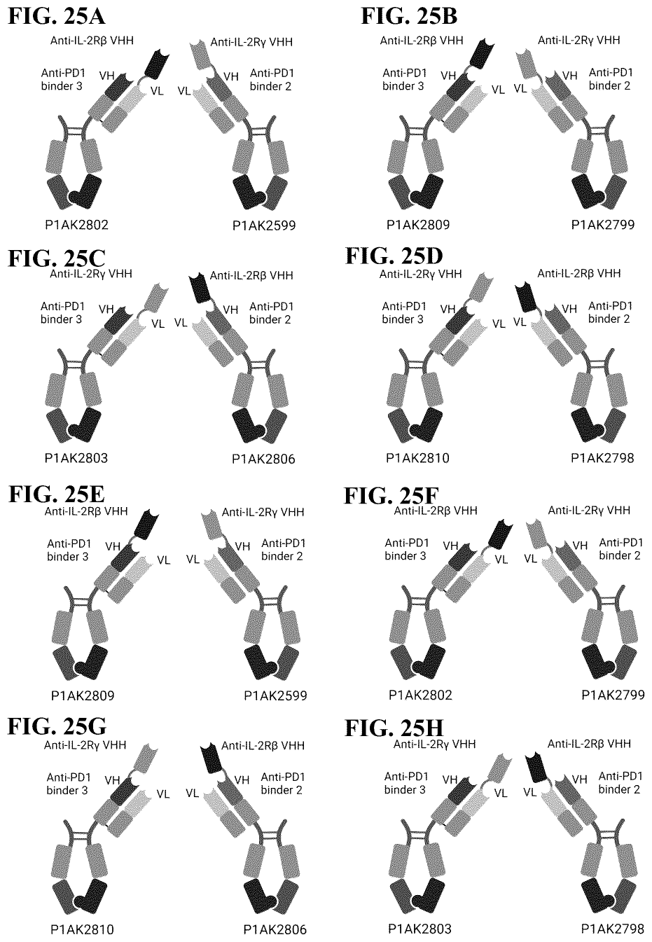

FIG. 25A-FIG. 25H. Concept of split PD-1-targeted IL-2R agonists with alternative anti-PD-1 Fab binders. VHH domains with IL-2Rβ and IL-2Rγ specificity were fused to the N-terminus of two different anti-PD-1 binding domains, via VH or VL fusion points, resulting in eight individual format configurations (P1AK2599; P1AK2798; P1AK2799; P1AK2802; P1AK2803; P1AK2806; P1AK2809; P1AK2810) that can be paired to produce the eight functional assemblies depicted: P1AK2802+P1AK2599 (FIG. 25A), P1AK2809+P1AK2799 (FIG. 25B), P1AK2803+P1AK2806 (FIG. 25C), P1AK2810+P1AK2798 (FIG. 25D), P1AK2809+P1AK2599 (FIG. 25E), P1AK2802 P1AK2 799 (FIG. 25F), P1AK2810+P1AK2806 (FIG. 25G) and P1AK2803+P1AK2798 (FIG. 25H).

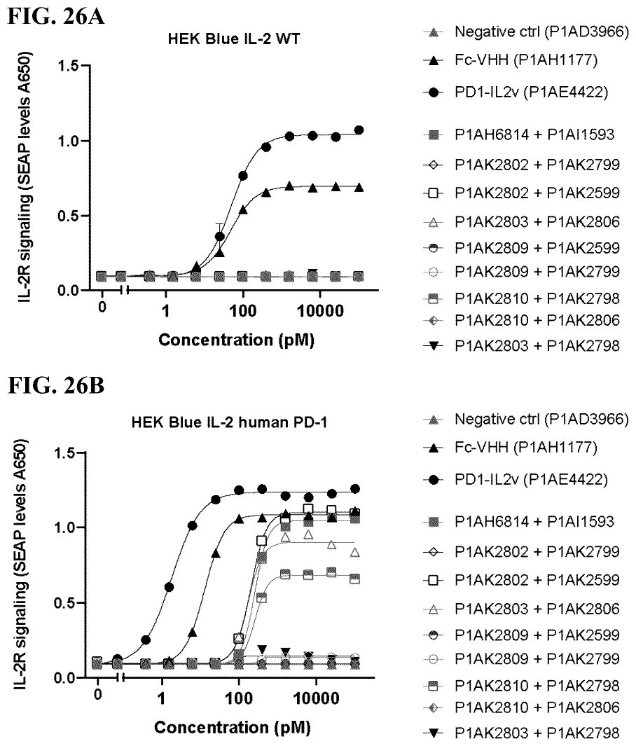

FIG. 26A-FIG. 26B. Functional activity of PD-1-targeted IL-2R agonists with alternative anti-PD-1 Fab binders. Test compounds were incubated with HEK Blue IL-2 wt cells (FIG. 26A) or HEK Blue IL-2 human PD-1 cells (FIG. 26B) for 21 h at 37° C. and 5% CO₂. PD1-IL2v (P1AE4422) and Fc-VHH (P1AH1177) were used as reference. A non-binding DP47 antibody (P1AD3966) was used as negative control. IL-2R signaling was measured via absorbance at 650 nm using QUANTI-Blue reagent. Shown are mean absorbance values+/−SEM of technical duplicates for each tested molecule concentration.

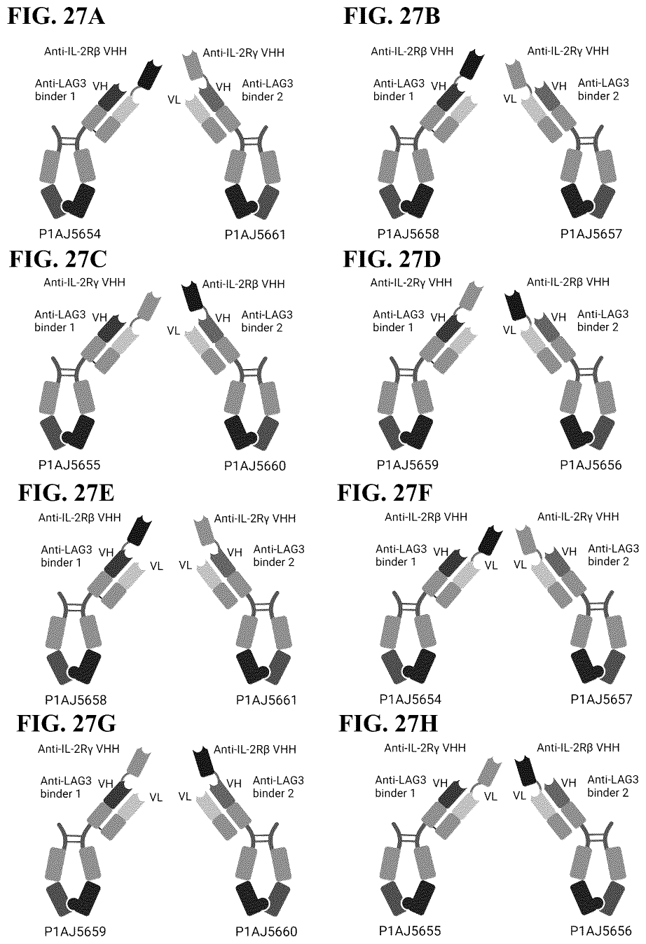

FIG. 27A-FIG. 27H. Concept of split LAG-3-targeted IL-2R agonists. VHH domains with IL-2Rβ and IL-2Rγ specificity were fused to the N-terminus of two different anti-LAG-3 binding domains, via VH or VL fusion points, resulting in eight individual format configurations (P1AJ5654−P1AJ5661) that can be paired to produce the eight functional assemblies depicted: P1AJ5654+P1AJ5661 (FIG. 27A), P1AJ5658+P1AJ5657 (FIG. 27B), P1AJ5655+P1AJ5660 (FIG. 27C), P1AJ5659+P1AJ5656 (FIG. 27D), P1AJ5658+P1AJ5661 (FIG. 27E), P1AJ5654+P1AJ5657 (FIG. 27F), P1AJ5659 (FIG. 27G) and P1AJ5655+P1AJ5656 (FIG. 27H).

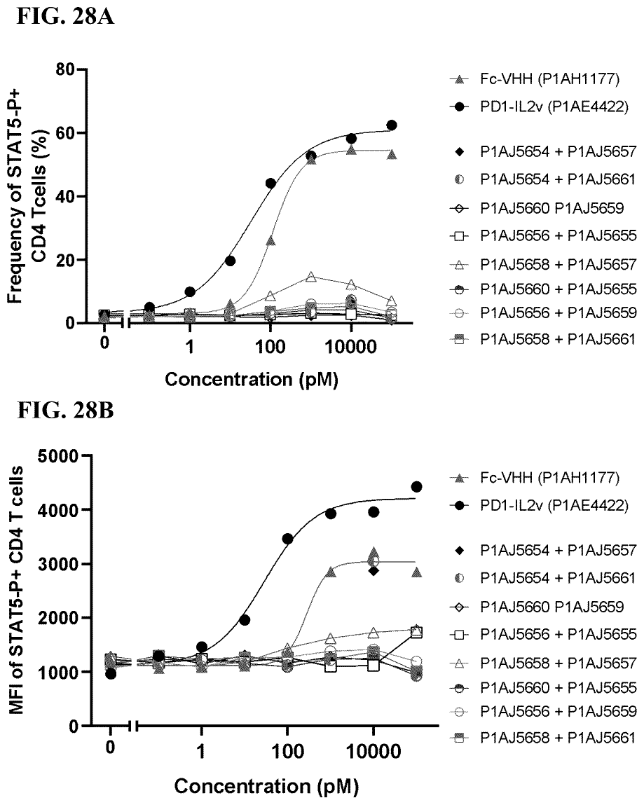

FIG. 28A-FIG. 28B. Functional activity of LAG-3-targeted IL-2R agonists. PD1-IL2v (P1AE4422) and Fc-VHH (P1AH1177) were used as reference. Frequency (FIG. 28A) and MFI (FIG. 28B) of STAT-5 phosphorylation were determined by flow cytometry after 60 min of test compounds co-incubation with CD4 T cells pre-activated for three days with plate-bound anti-CD3 and soluble anti-CD28 antibodies.

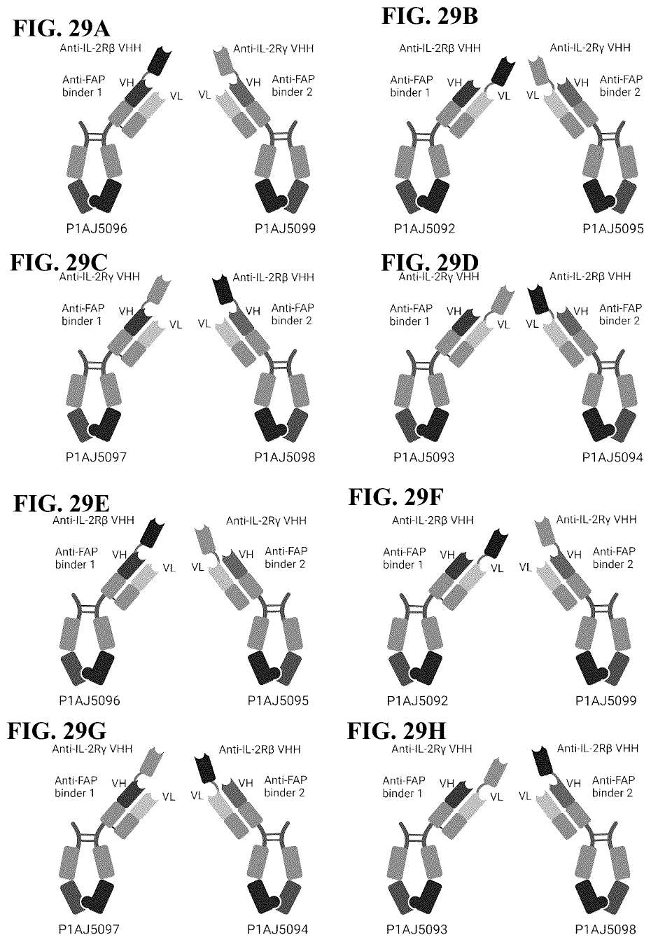

FIG. 29A-FIG. 29H. Concept of split FAP-targeted IL-2R agonists. VHH domains with IL-2Rβ and IL-2Rγ specificity were fused to the N-terminus of two different anti-FAP binding domains, via VH or VL fusion points, resulting in eight individual format configurations (P1AJ5092−P1AJ5099) that can be paired to produce the eight functional assemblies: P1AJ5096+P1AJ5099 (FIG. 29A), P1AJ5092+P1AJ5095 (FIG. 29B), P1AJ5097+P1AJ5098 (FIG. 29C), P1AJ5093+P1AJ5094 (FIG. 29D), P1AJ5096+P1AJ5095 (FIG. 29E), P1AJ5092+P1AJ5099 (FIG. 29F), P1AJ5097+P1AJ5094 (FIG. 29G) and P1AJ5093+P1AJ5098 (FIG. 29H).

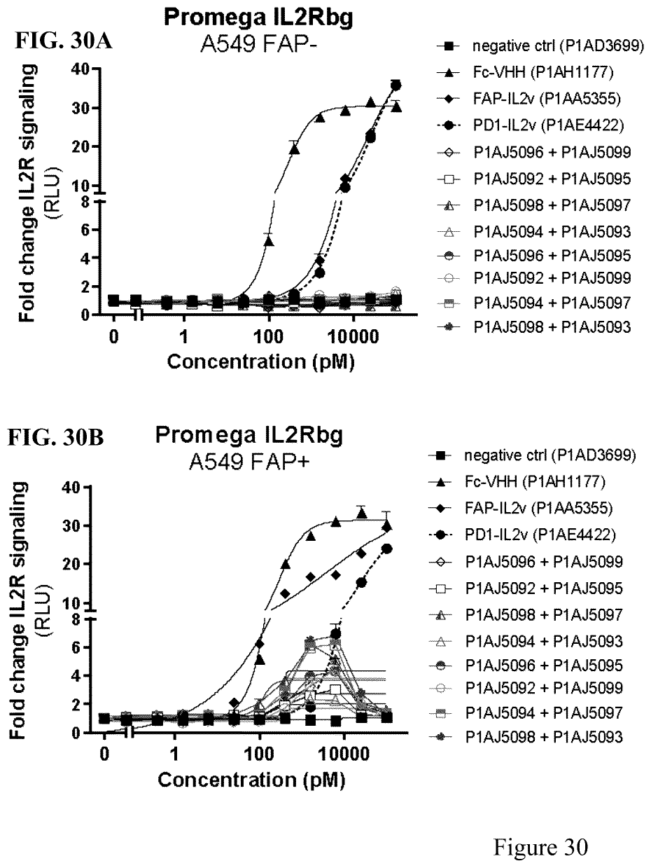

FIG. 30A-FIG. 30B. Functional activity of FAP-targeted IL-2R agonists. Test compounds were incubated with A549 FAP-negative target cells (FIG. 30A) or A549 FAP-positive target cells (FIG. 30B) and IL-2Rβγ Bioassay cells for 20 h at 37° C. and 5% CO₂. PD1-IL2v (P1AE4422), FAP-IL2v (P1AA5355) and Fc-VHH (P1AH1177) were used as reference. A non-binding DP47 antibody (P1AD3966) was used as negative control. IL-2R signaling was measured via luminescence using Bio-Glo NL reagent and normalized to background signal. Shown are mean fold change RLU values+/−SEM of technical duplicates for each tested molecule concentration.

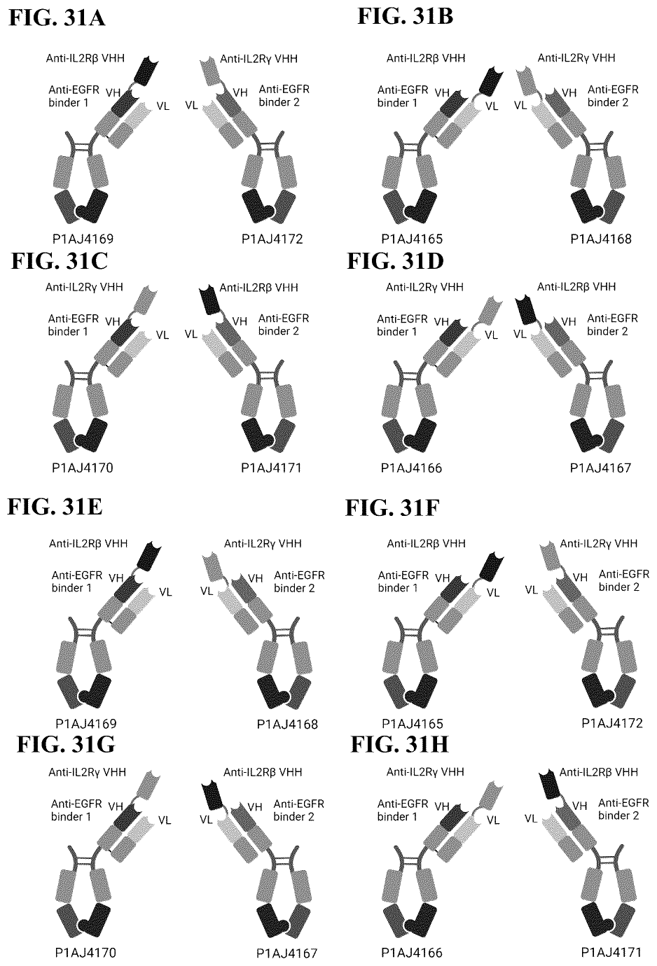

FIG. 31A-FIG. 31H. Concept of split EGFR-targeted IL-2R agonists. VHH domains with IL-2Rβ and IL-2Rγ specificity were fused to the N-terminus of two different anti-EGFR binding domains, via VH or VL fusion points, resulting in eight individual format configurations (P1AJ4165−P1AJ4172) that can be paired to produce the eight functional assemblies: P1AJ4169+P1AJ4172 (FIG. 31A), P1AJ4165+P1AJ4168 (FIG. 31B), P1AJ4170+P1AJ4171 (FIG. 31C), P1AJ4166+P1AJ4167 (FIG. 31D), P1AJ4169+P1AJ4168 (FIG. 31E), P1AJ4165+P1AJ4172 (FIG. 31F), P1AJ4170+P1AJ4167 (FIG. 31G) and P1AJ4166+P1AJ4171 (FIG. 31H).

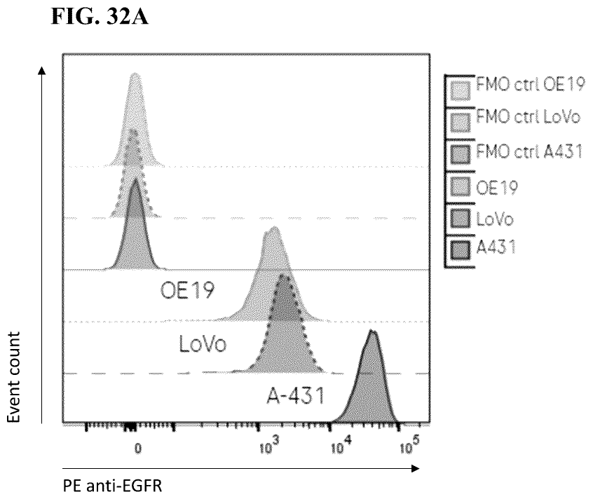

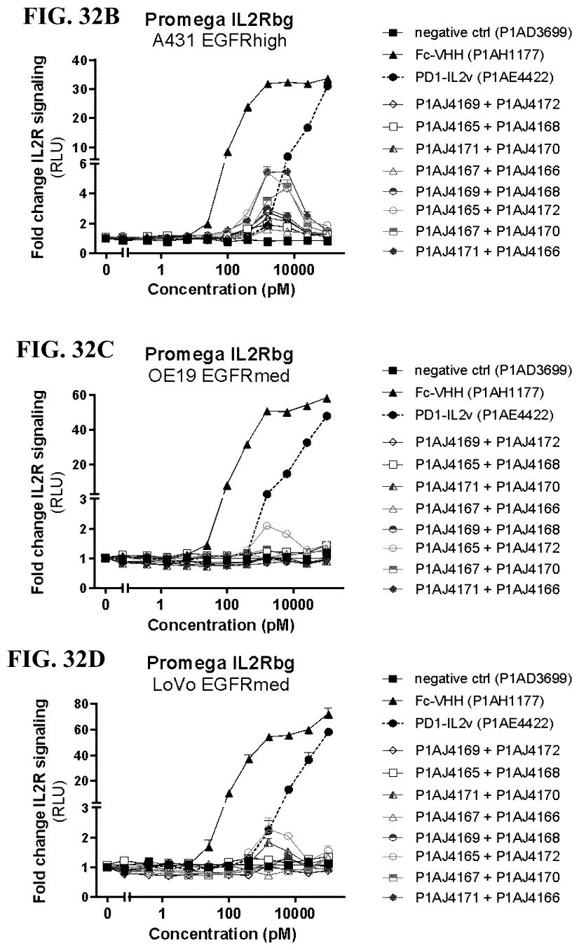

FIG. 32A-FIG. 32D. Functional activity of EGFR-targeted IL-2R agonists. EGFR expression on target cells by flow cytometry (FIG. 32A). Test compounds were incubated with A431 target cells (FIG. 32B), OE19 target cells (FIG. 32C) or LoVo target cells (FIG. 32D)) and IL-2Rβγ Bioassay cells for 20 h at 37° C. and 5% CO₂. PD1-IL2v (P1AE4422) and Fc-VHH (P1AH1177) were used as reference. A non-binding DP47 antibody (P1AD3966) was used as negative control. IL-2R signaling was measured via luminescence using Bio-Glo NL reagent and normalized to background signal. Shown are mean fold change RLU values+/−SEM of technical duplicates for each tested molecule concentration.

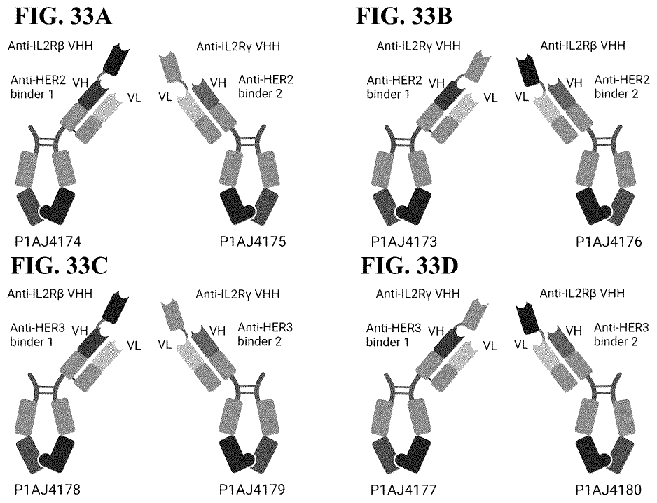

FIG. 33A-FIG. 33D. Concept of split HER2-targeted and HER3-targeted IL-2R agonists. VHH domains with IL-2Rβ and IL-2Rγ specificity were fused to the N-terminus of two different anti-HER2 binding domains and two different anti-HER3 binding domains. VH or VL fusion points were selected for each Fab based on structural data. Four format configurations (P1AJ4173−P1AJ4176) and two paired assemblies were designed for HER2: P1AJ4174+P1AJ4175 (FIG. 33A) and P1AJ4173+P1AJ4176 (FIG. 33B). Four format configurations (P1AJ4177−P1AJ4180) and two paired assemblies were selected for HER3: P1AJ4178+P1AJ4179 (FIG. 33C) and P1AJ4177+P1AJ4180 (FIG. 33D).

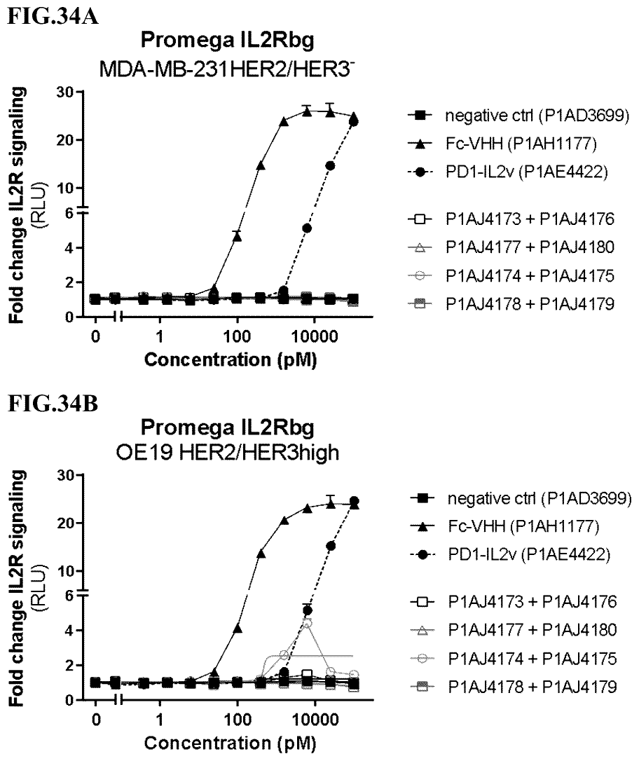

FIG. 34A-FIG. 34B. Functional activity of HER2-targeted and HER3-targeted IL-2R agonists. Test compounds were incubated with MDA-MB-231 target cells (FIG. 34A) or OE19 target cells (FIG. 34B) and IL-2Rβγ Bioassay cells for 20 h at 37° C. and 5% CO₂. PD1-IL2v (P1AE4422) and Fc-VHH (P1AH1177) were used as reference. A non-binding DP47 antibody (P1AD3966) was used as negative control. IL-2R signaling was measured via luminescence using Bio-Glo NL reagent and normalized to background signal. Shown are mean fold change RLU values+/−SEM of technical duplicates for each tested molecule concentration.

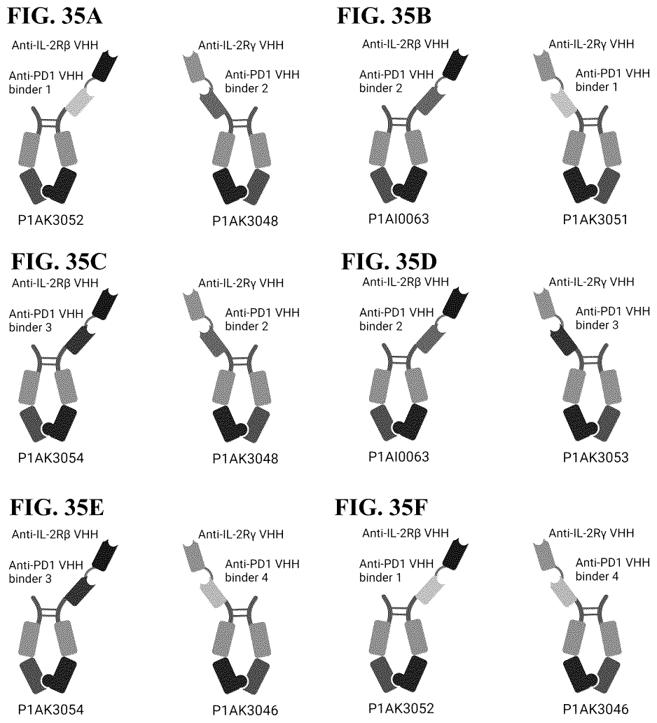

FIG. 35A-FIG. 35F. Concept of split PD-1-targeted IL-2R agonists with alternative anti-PD-1 single domain binders. VHH domains with IL-2Rβ and IL-2Rγ specificity were fused to the N-terminus of four different anti-PD-1 single domain binders (P1AI0063; P1AK3046; P1AK3048; P1AK3051−P1AK3054) that can be paired to produce the six functional assemblies depicted: P1AK3052+P1AK3048 (FIG. 35A), P1AI0063+P1AK3051 (FIG. 35B), P1AK6054+P1AK3048 (FIG. 35C), P1AI0063+P1AK3053 (FIG. 35D), P1AK3054+P1AK3046 (FIG. 35E) and P1AK3052+P1AK3046 (FIG. 35F).

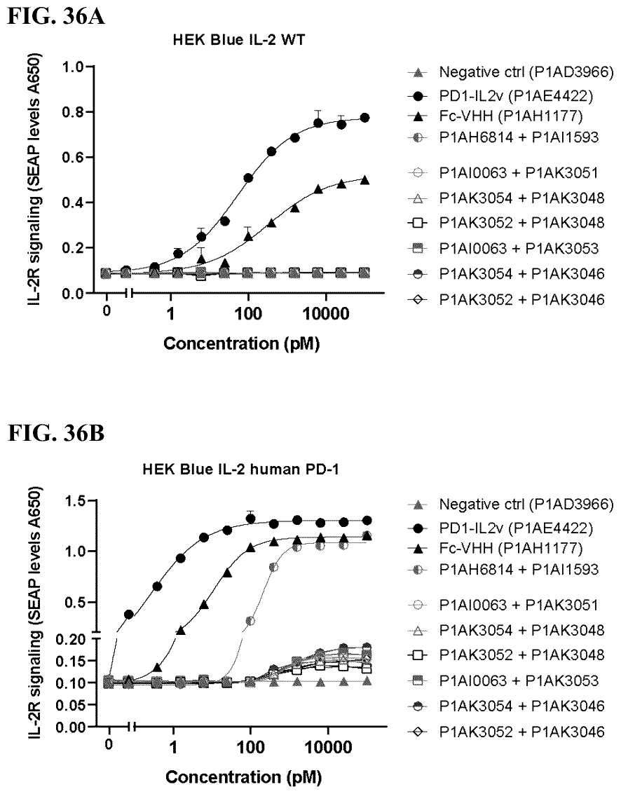

FIG. 36A-FIG. 36B. Functional activity of PD-1-targeted IL-2R agonists with alternative anti-PD-1 single domain binders. Test compounds were incubated with HEK Blue IL-2 wt cells (FIG. 36A) or HEK Blue IL-2 human PD-1 cells (FIG. 36B) for 20 h at 37° C. and 5% CO₂. PD1-IL2v (P1AE4422) and Fc-VHH (P1AH1177) were used as reference. A non-binding DP47 antibody (P1AD3966) was used as negative control. IL-2R signaling was measured via absorbance at 650 nm using QUANTI-Blue reagent. Shown are mean absorbance values+/−SEM of technical duplicates for each tested molecule concentration.

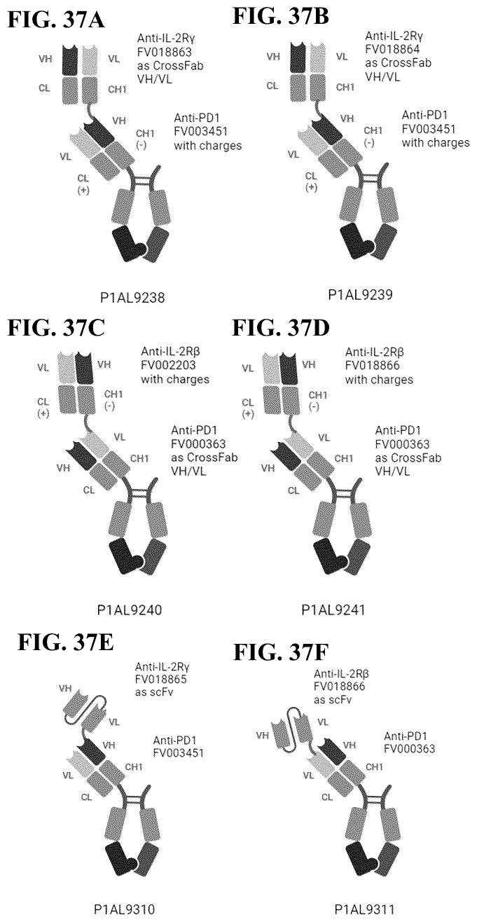

FIG. 37A-FIG. 37F. Concept of split PD-1-targeted IL-2R agonists with anti-IL2R Fab binders or anti-IL2R scFv binders. Fab fragments with IL-2Rβ and IL-2Rγ specificity were fused to the N-termini of two anti-PD1 binding domains with different paratopes, via VH or VL fusion points. P1AL9238 (FIG. 37A) and P1AL9239 (FIG. 37B) comprise the PD1 binder 2 (FV003451 paratope; 1040 binder) and two different IL2Rγ binders, FV018863 and FV018864, respectively, whereas P1AL9240 (FIG. 37C) and P1AL9241 (FIG. 37D) comprise the PD1 binder 1 (FV000363 paratope; 0376 binder) and two different IL2Rβ binders, FV002203 and FV018866, respectively. These can be paired to produce the four functional assemblies: P1AL9238+P1AL9240, P1AL9238+P1AL9241, P1AL9239+P1AL9240, P1AL9239+P1AL9241. Alternatively, scFv fragments with IL-2Rβ and IL-2Rγ specificity were fused to the N-termini of two anti-PD1 binding domains with different paratopes, via VH or VL fusion points. P1AL9310 (FIG. 37E) comprises the PD1 binder 2 (FV003451 paratope) and the IL2Rγ binder FV018865, whereas in P1AL9311 (FIG. 37F) comprises the PD1 binder 1 (FV000363 paratope) and the IL2Rβ binder FV018866. These can be paired to produce a functional assembly: P1AL9310+P1AL9311.

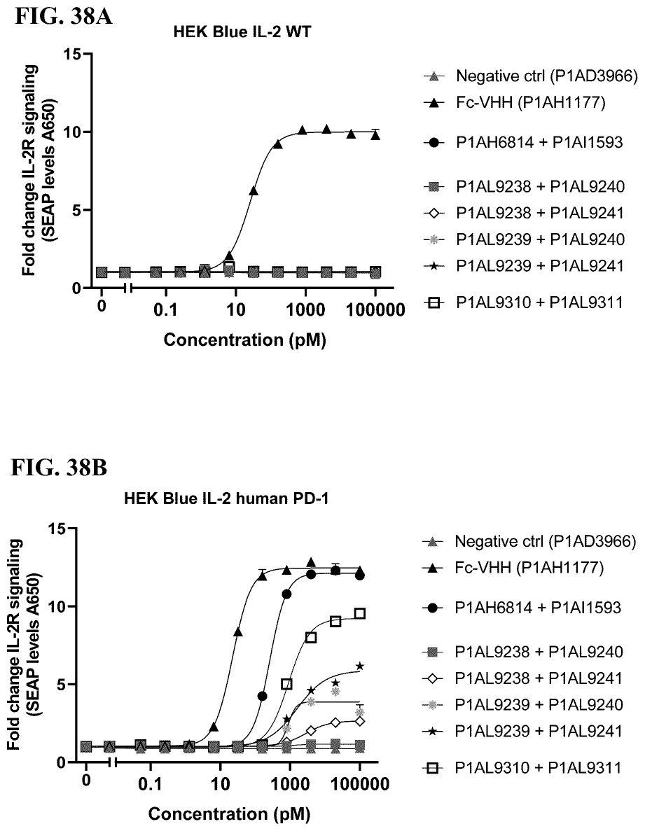

FIG. 38A-FIG. 38B. Functional activity of PD-1-targeted IL-2R agonists with anti-IL2R Fab and scFv binders. Test compounds were incubated with HEK Blue IL-2 wt cells (FIG. 38A) or HEK Blue IL-2 human PD-1 cells (FIG. 38B) for 18 h at 37° C. and 5% CO₂. An IL-2 agonistic heavy chain antibody (Fc-VHH; P1AH1177) was used as reference. A non-binding DP47 antibody (P1AD3966) was used as negative control. IL-2R signaling was measured via absorbance at 650 nm using QUANTI-Blue reagent. Shown are fold change in IL-2R signaling over background+/−SEM of technical duplicates for each tested molecule concentration.

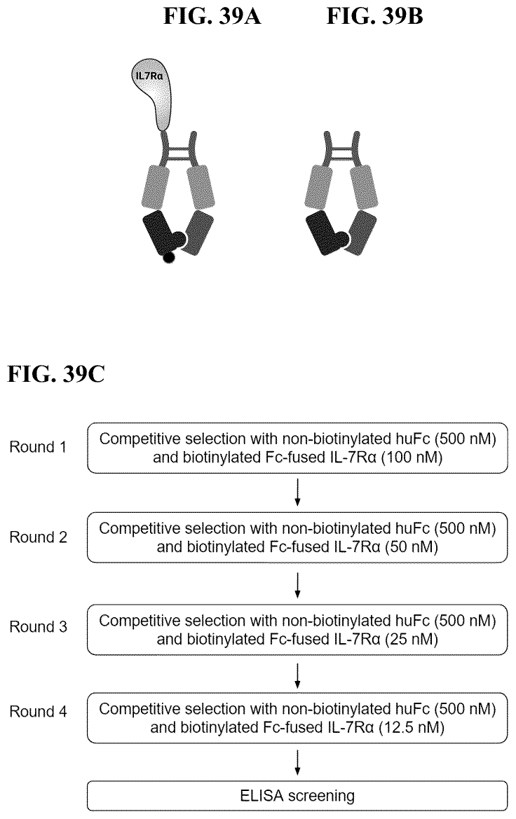

FIG. 39A-FIG. 39C. Schematic representations of antigens and workflow for the isolation of IL-7Rα-specific single domain antibodies. The extracellular domain (ECD) of human IL-7Rα fused to biotinylated Fc knob-into-hole (P1AI1009 represented in FIG. 39A) was generated for alpaca immunization, phage display and screening. Soluble human Fc knob-into-hole (P1AD4290 represented in FIG. 39B) was generated for phage display. Workflow for the enrichment of single domain antibodies with binding specificity for IL-7Rα by phage display (FIG. 39C).

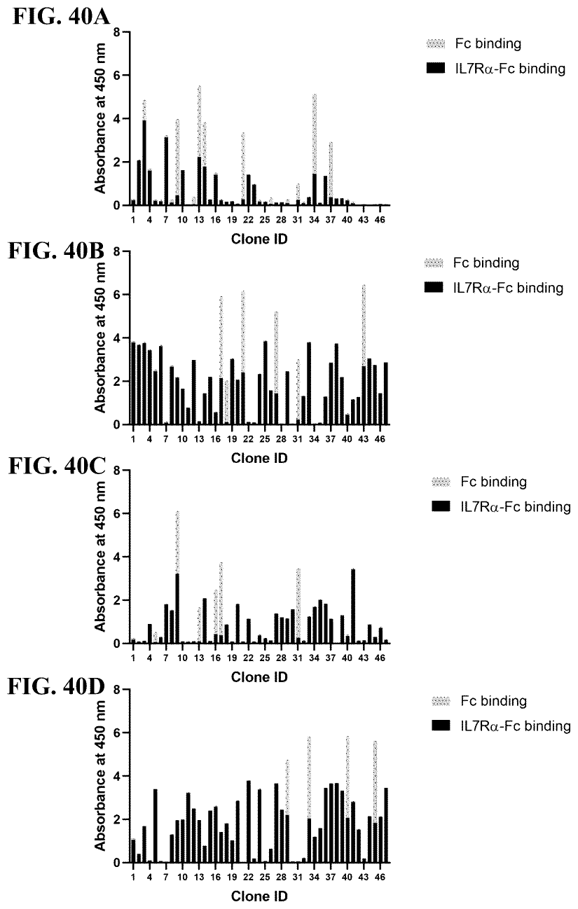

FIG. 40A-FIG. 40D. Specificity screening of single domain antibodies by ELISA. Soluble VHH domains enriched for IL-7Rα specificity were randomly selected and screened for binding to immobilized human IL-7Rα-Fc and immobilized human Fc. Following three rounds of phage display, VHH from library 1 (FIG. 40A) and library 2 (FIG. 40B) were tested for antigen specificity. Following four rounds, enriched VHH from library 1 (FIG. 40C) and library 2 (FIG. 40D) were also tested for specificity by ELISA. Absorbance at 450 nm indicating the binding response to each target is represented as a stacked bar chart.

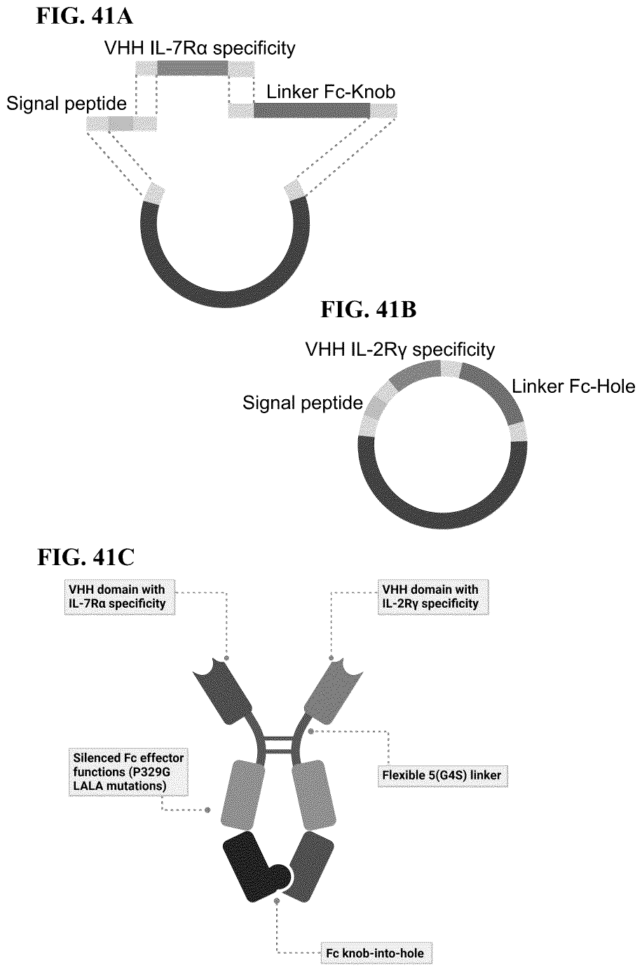

FIG. 41A-FIG. 41C. Format conversion of single domain antibody fragments into bispecific heavy chain antibodies. Round 3 and round 4 IL-7Rα-specific VHH from libraries 1 and 2 were fused to Fc-Knob and cloned into a mammalian cell expression vector using Gibson cloning method (FIG. 41A). Previously identified VHH with IL-2Rγ specificity were fused to Fc-hole using the same method (FIG. 41B). Bispecific heavy chain antibodies comprising IL-7Rα-IL-2Rγ VHH domain pairs were generated via knob-into-hole assembly of Fc chains and flexible 5(G4S) linkers to fuse VHH moieties to Fc chains. Effector functions of the Fc were silenced via incorporation of P329G LALA mutations in the CH2 domain (FIG. 41C).

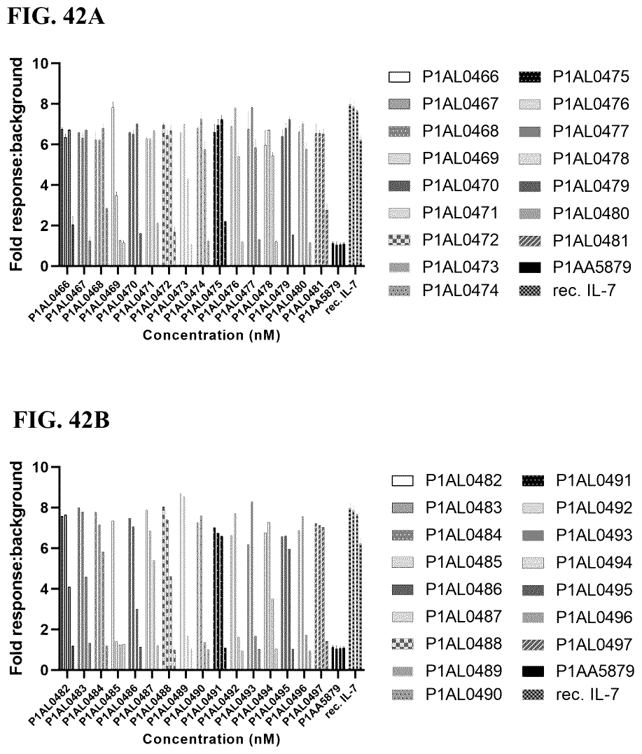

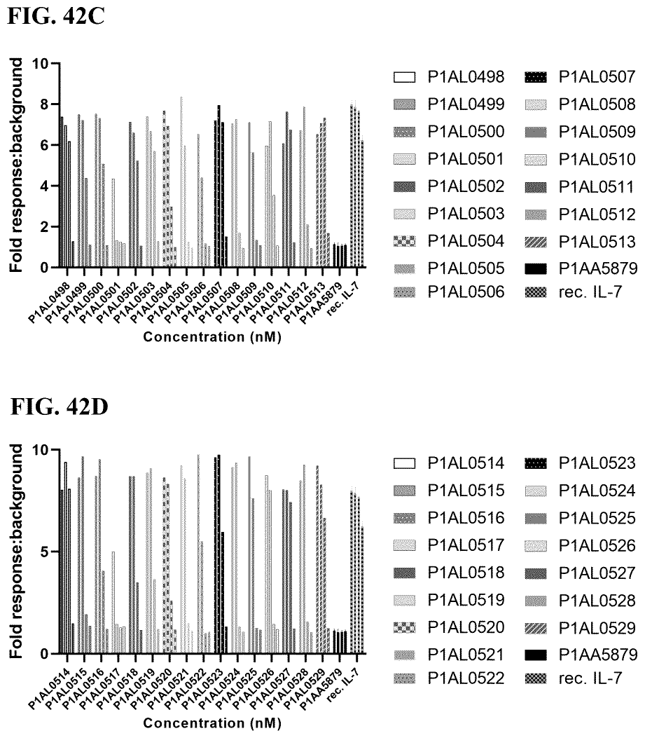

FIG. 42A-FIG. 42D. Functional characterization of dose-dependent response for IL-7R agonists. Bispecific heavy chain antibodies comprising IL-7Rα specificity and IL-2Rγ were screened for dose-dependent IL-7R agonism in HEK Blue IL-7 cells. Recombinant IL-7 was used as a reference and an IgG with irrelevant specificity was used as a negative control (P1AA5879). Each molecule was incubated with reporter cells at the following concentrations: 20 nM, 2 nM, 0.2 nM, 0.02 nM shown from left to right. IL-7R activity was quantified by absorbance at 650 nm and fold response/background is shown for each treatment. Graphs were separated according to the four different IL-2Rγ-specific VHH clones: IL2Rγ_2 (A), IL2Rγ_3 (B), IL2Rγ_4 (FIG. 42C) and IL2Rγ_5 (FIG. 42D).

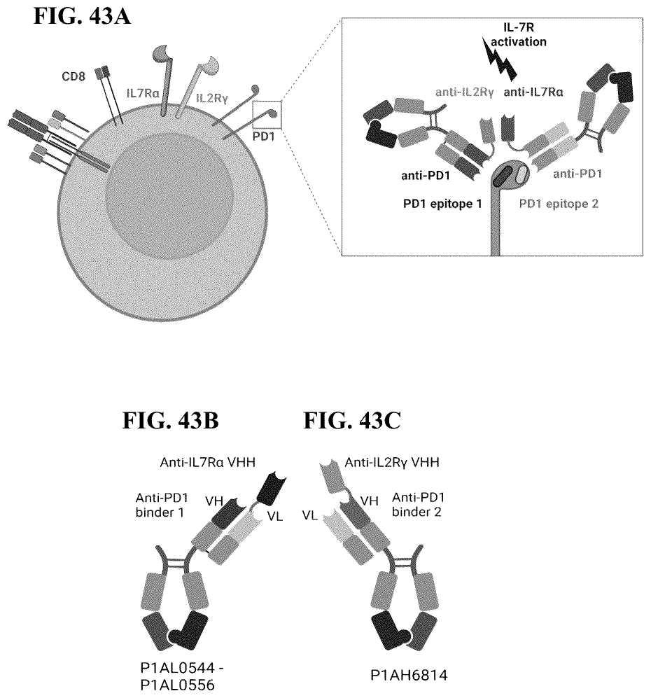

FIG. 43A-FIG. 43C. Concept of split PD-1-targeted IL-7R agonists. PD-1-dependent biparatopic assembly of split IL-7 mimetics (FIG. 43A). VHH domains with IL-7Rα and IL-2Rγ specificity were fused to the N-terminus of two different anti-PD-1 binding domains, via VH or VL fusion points: IL-7Rα-VHH were fused to the VL of PD-1 binder 1 (FIG. 43B), and IL-2Rγ-VHH were fused to the VH of PD-1 binder 2 (FIG. 43C). IL-7Rα molecules contained AAA mutations (I253A; H310A; H435A, numbering according to Kabat EU index) in the CH2 and CH3 domains of the Fc hole to facilitate protein A purification. Effector functions of the Fc were silenced in all molecules via incorporation of P329G LALA mutations in the CH2 domain.

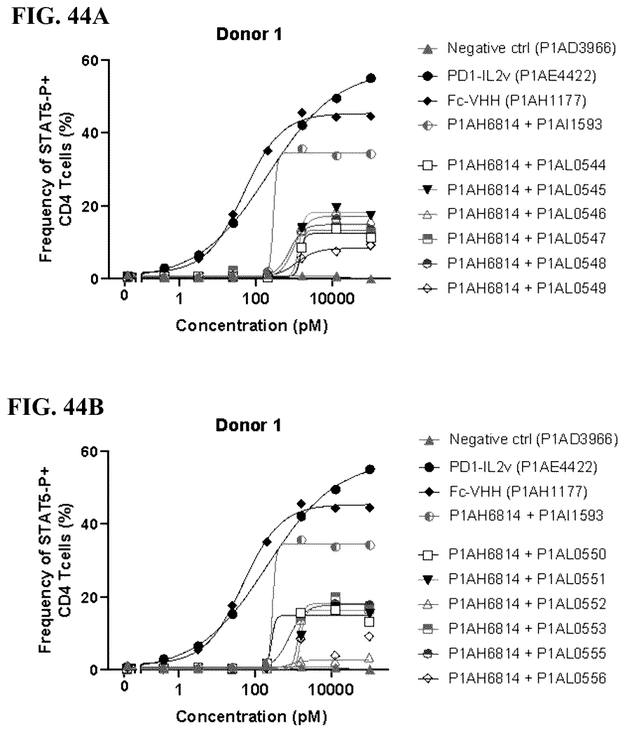

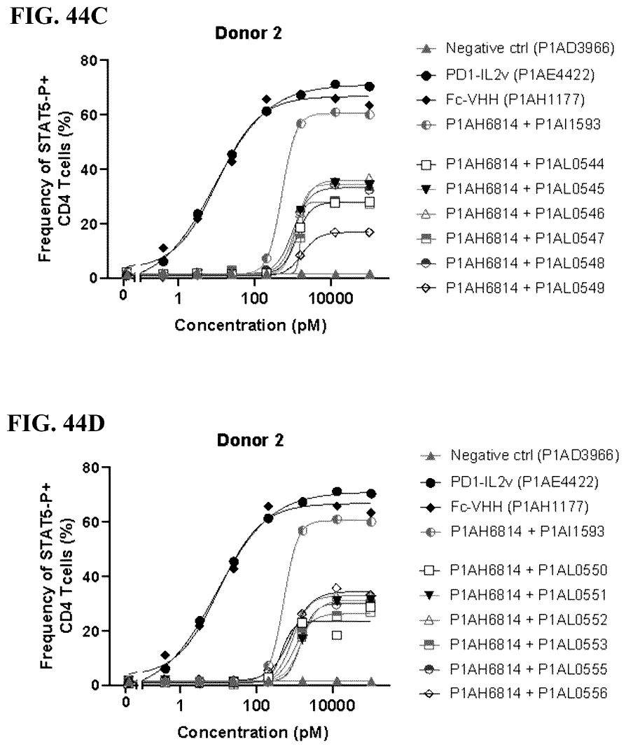

FIG. 44A-FIG. 44D. Functional activity of PD-1-targeted IL-7R agonists. PD1-IL2v (P1AE4422) and Fc-VHH (P1AH1177) were used as reference. A non-binding DP47 antibody (P1AD3966) was used as negative control. Frequency of STAT-5 phosphorylation was determined by flow cytometry after 60 min of test compounds co-incubation with CD4 T cells pre-activated for three days with plate-bound anti-CD3 and soluble anti-CD28 antibodies for blood donor 1 (FIG. 44A and FIG. 44B) and 2 (FIG. 44C and FIG. 44D).

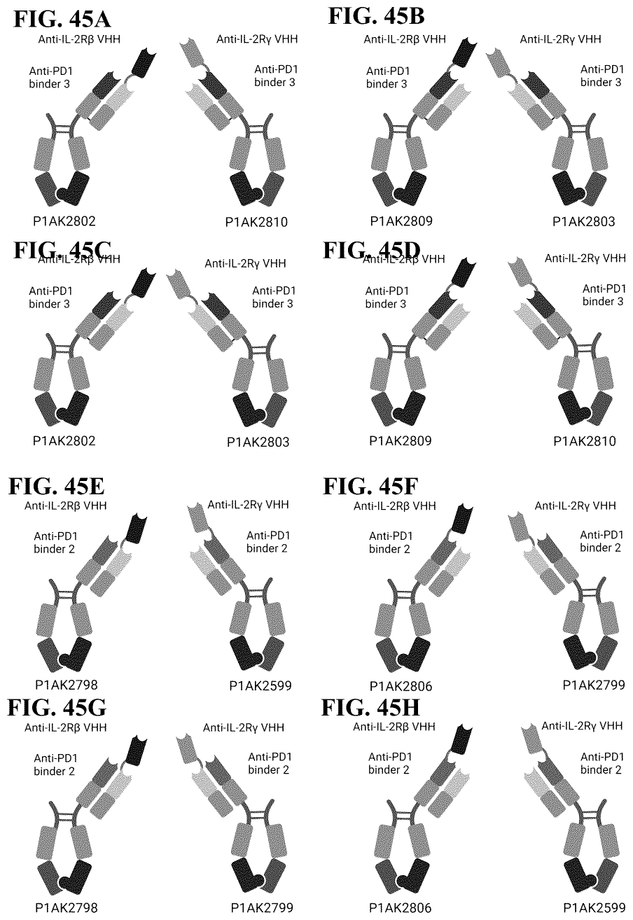

FIG. 45A-FIG. 45H. Concept of split PD-1-targeted IL-2R agonists with monoparatopic assembly onto PD-1. VHH domains with IL-2Rβ and IL-2Rγ specificity were fused to the N-terminus of two different anti-PD-1 binding domains, via VH or VL fusion points, resulting in eight individual format configurations of IL-2R agonist pairs containing the same anti-PD-1 Fab binders (monoparatopic assembly) that can be paired to produce the eight monoparatopic assemblies: P1AK2802+P1AK2810 (FIG. 45A), P1AK2809+P1AK2803 (FIG. 45B), P1AK2802+P1AK2803 (FIG. 45C), P1AK2809+P1AK2810 (FIG. 45D), P1AK2798+P1AK2599 (FIG. 45E), P1AK2806+P1AK2799 (FIG. 45F), P1AK2798+P1AK2799 (FIG. 45G) and P1AK2806+P1AK2599 (FIG. 45H).

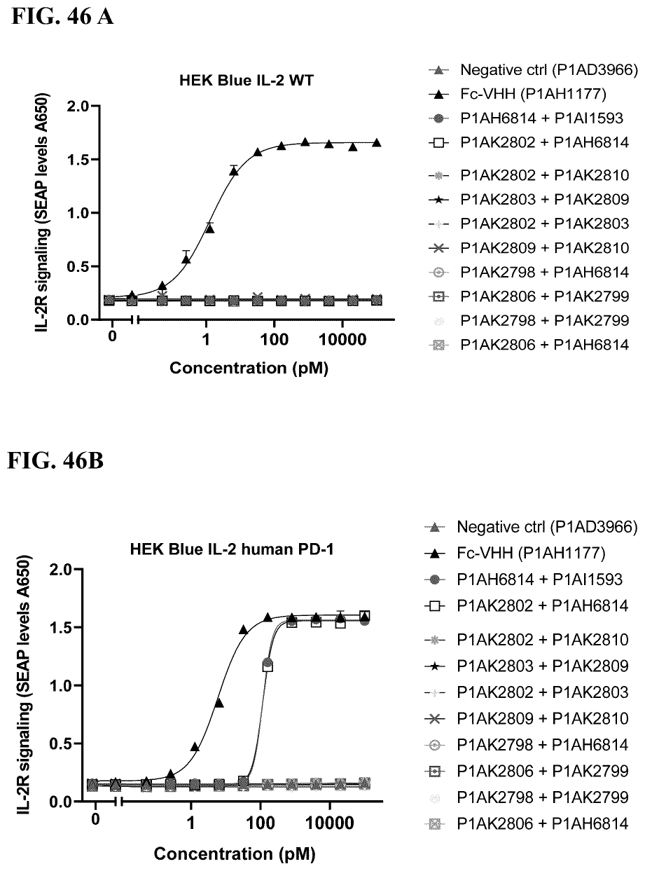

FIG. 46A-FIG. 46B. Functional activity of monoparatopic vs biparatopic PD-1-targeted IL-2R agonists. Test compounds were incubated with HEK Blue IL-2 wt (FIG. 46A) or HEK Blue IL-2 human PD-1 cells (FIG. 46B) for 21 h at 37° C. and 5% CO₂. Two biparatopic PD-1-targeted IL-2R agonists (P1AH6814+P1AI1593 and P1AH6814+P1AK2802) and the IL-2 mimetic heavy chain antibody (Fc-VHH; P1AH1177) were used as reference. A non-binding DP47 antibody (P1AD3966) was used as negative control. IL-2R signaling was measured via absorbance at 650 nm using QUANTI-Blue reagent. Shown are mean absorbance values+/−SEM of technical duplicates for each tested molecule concentration.

### DETAILED DESCRIPTION OF THE INVENTION

### Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The methods and techniques of the present disclosure are generally performed according to conventional methods well known in the art. Generally, nomenclatures used in connection with, and techniques of biochemistry, enzymology, molecular, and cellular biology, microbiology, genetics and protein and nucleic acid chemistry and hybridization described herein are those well-known and commonly used in the art.

The terms “a”, “an” and “the” generally include plural referents, unless the context clearly indicates otherwise.

As used herein, the terms “first”, “second”, “third” or “fourth” with respect to binding molecules, epitopes, polypeptides etc., are used for convenience of distinguishing when there is more than one of each type of moiety. Use of these terms is not intended to confer a specific order or orientation of the moiety unless explicitly so stated.

The term “antigen binding molecule” as used herein refers to a polypeptide molecule (composed of one or more polypeptide chains) that is capable of binding to an antigen. An antigen binding molecule may be derived from an antibody, and typically comprises an antigen binding domain.

The term “antibody” herein is used in the broadest sense and encompasses various antibody structures, including but not limited to monoclonal antibodies, polyclonal antibodies, multispecific antibodies (e.g. bispecific antibodies), heavy-chain antibodies, antibody fragments and antigen binding molecules so long as they exhibit the desired antigen-binding activity.

The term “heavy chain antibody” and “heavy chain-only antibody” and “HCAb” as used herein refer to antibodies devoid of light chains.

The terms “full-length antibody,” “intact antibody,” and “whole antibody” are used herein interchangeably to refer to an antibody having a structure substantially similar to a native antibody structure.

An “antibody fragment” refers to a molecule other than an intact antibody that comprises a portion of an intact antibody that binds the antigen to which the intact antibody binds. Examples of antibody fragments include but are not limited to Fv, Fab, cross-Fab, Fab′, Fab′-SH, F(ab′)2, diabodies, linear antibodies, single-chain antibody molecules (e.g. scFv and scFab), single-domain antibodies, and multispecific antibodies formed from antibody fragments. For a review of certain antibody fragments, see Hollinger and Hudson, Nature Biotechnology 23:1126-1136 (2005).

A “single-domain antibody” refers to an antibody fragment consisting of a single monomeric antibody variable domain such as VHHs, nanobodies, VNARs derived from sharks, autonomous VH domains or autonomous VL domains. Single-domain antibodies provide an antigen-binding site which specifically binds to an epitope, i.e. the antigen binding-site is formed solely by the single-domain antibody.

A “VHH” or “VHH domain” or “nanobody” refers to a single-domain antibody derived from the variable domains of heavy chain antibodies from camelids, e.g. camel, dromedary, llama, alpaca, etc. (See Nguyen V. K. et al., 2000, The EMBO Journal, 19, 921-930; Muyldermans S., 2001, J Biotechnol., 74, 277-302 and for review Vanlandschoot P. et al., 2011, Antiviral Research 92, 389-407). The antigen-binding site of VHHs is devoid of light chain variable domain. A VHH domain may be humanized.

An “antigen binding domain” as used herein refers to a domain that specifically binds to a target antigen. The term in particular refers to an antigen binding domain of an antibody, i.e. the part that comprises the area which binds to and is complementary to part or all of an antigen. Accordingly, in particular aspects, an antigen binding domain herein is an antigen binding domain of an antibody. Such an antigen binding domain may be provided by an antibody fragment, for example by a Fab molecule, a single-chain antibody molecule or single-domain antibodies, such as a VHH domain.

The term “variable region” or “variable domain” refers to the domain of an antibody heavy or light chain that is involved in binding the antibody to the antigen. The term includes VHH domains. The variable domains of the heavy chain and light chain (VH and VL, respectively) of a native antibody generally have similar structures, with each domain comprising four conserved framework regions (FRs) and complementarity determining regions (CDRs). See, e.g., Kindt et al., Kuby Immunology, 6th ed., W.H. Freeman & Co., page 91 (2007). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991). As used herein in connection with variable region sequences, “Kabat numbering” refers to the numbering system set forth by Kabat et al., Sequences of Proteins of Immunological Interest, 5th Ed. Public Health Service, National Institutes of Health, Bethesda, MD (1991).

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, MD (1991), referred to as “numbering according to Kabat” or “Kabat numbering” herein.

Specifically the Kabat numbering system (see pages 647-660 of Kabat, et al., Sequences of Proteins of Immunological Interest, 5th ed., Public Health Service, National Institutes of Health, Bethesda, MD (1991)) is used for the light chain constant domain CL of kappa and lambda isotype and the Kabat EU index numbering system (see pages 661-723) is used for the heavy chain constant domains (CH1, hinge, CH2 and CH3), which is herein further clarified by referring to “numbering according to Kabat EU index” or “Kabat EU index numbering” in this case.

The terms “binding site” or “antigen-binding site” as used herein refers to the site, i.e. one or more amino acid residues, of a binding molecule which provides interaction with the antigen. For example, the antigen binding site of an antigen binding domain comprises amino acid residues from the complementarity determining regions (CDRs). An antigen-binding site may be provided by, for example, one or more variable domains (also called variable regions). In single domain antibodies the antigen-binding site is provided by a single variable domain. Whereas, in a Fab fragment the antigen-binding site is provided by the VH and VL domains.

The term “hypervariable region” or “HVR”, as used herein, refers to each of the regions of an antigen binding domain which are hypervariable in sequence and which determine antigen binding specificity, for example “complementarity determining regions” (“CDRs”). Generally, variable domains comprise three CDRs. Thus antibodies comprising a VH and a VL comprise six CDRs; three in the VH (HCDR1, HCDR2, HCDR3), and three in the VL (LCDR1, LCDR2, LCDR3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than complementarity determining regions (CDRs). The FR of a variable domain generally consists of four FR domains: FR1, FR2, FR3, and FR4. Accordingly, the HVR and FR sequences generally appear in the following order in VH and VHH domains (or VL): FR1-HCDR1 (LCDR1)-FR2-HCDR2 (LCDR2)-FR3-HCDR3 (LCDR3)-FR4. Unless otherwise indicated, CDR residues and other residues in the variable domain (e.g., FR residues) are numbered herein according to Kabat et al., supra.

The term “immunoglobulin molecule” herein refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable domain (VH), also called a variable heavy domain or a heavy chain variable region, followed by three constant domains (CH1, CH2, and CH3), also called a heavy chain constant region. Similarly, from N- to C-terminus, each light chain has a variable domain (VL), also called a variable light domain or a light chain variable region, followed by a constant light (CL) domain, also called a light chain constant region. The heavy chain of an immunoglobulin may be assigned to one of five types, called α (IgA), δ (IgD), ε (IgE), γ (IgG), or μ (IgM), some of which may be further divided into subtypes, e.g. γ1 (IgG₁), γ2 (IgG₂), γ3 (IgG₃), γ4 (IgG₄), α1 (IgA1) and α2 (IgA2). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA1, and IgA2. The heavy chain constant domains that correspond to the different classes of immunoglobulins are called α, δ, ε, γ, and μ, respectively.

A “Fab molecule” or “Fab” or “Fab fragment” or “Fab domain” refers to a protein consisting of the VH and CH1 domain of the heavy chain (the “Fab heavy chain”) and the VL and CL domain of the light chain (the “Fab light chain”) of an immunoglobulin.

A “cross-Fab molecule” or “cross-Fab” or “crossover Fab molecule” refers to a Fab molecule, wherein either the variable regions or the constant regions of the heavy and light chain are exchanged. Cross-Fab engineering enables two different chain compositions of a cross-Fab molecules. On the one hand, the variable regions of the Fab heavy and light chain are exchanged, i.e. the cross-Fab molecule comprises a peptide chain composed of the light chain variable region (VL) and the heavy chain constant region (CH1), wherein the CH1 may be fused to an Fc domain, and a peptide chain composed of the heavy chain variable region (VH) and the light chain constant region (CL). On the other hand, when the constant regions of the Fab heavy and light chain are exchanged, the cross-Fab molecule comprises a peptide chain composed of the heavy chain variable region (VH) and the light chain constant region (CL), wherein the CL may be fused to an Fc domain, and a peptide chain composed of the light chain variable region (VL) and the heavy chain constant region (CH1).

The term “conventional Fab molecule” refers to a Fab molecule composed of a Fab heavy chain comprising a VH and CH1 domain and a Fab light chain comprising a VL and CL domain.

A “single-chain variable fragment” or “scFv” molecule is a fusion protein of the variable domains of the heavy (VH) and light chain (VL) of an antibody, connected by a linker. In particular, the linker is a short polypeptide of 10 to 25 amino acids and is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker.

The term “multispecific” means that a binding molecule (e.g. an antibody) is able to specifically bind to at least two distinct antigens. A multispecific binding molecule (e.g. antibody) can be, for example, a bispecific binding molecule. Typically, a bispecific binding molecule comprises two antigen binding sites, each of which is specific for a different antigens. In certain aspects, the multispecific (e.g. bispecific) binding molecule is capable of simultaneously binding two antigens, particularly two antigens expressed on the same cell, on neighbouring cells, or cells in the same tissue.

The term “valent” as used herein denotes the presence of a specified number of antigen binding sites in a binding molecule. As such, the term “monovalent binding to an antigen” denotes the presence of one (and not more than one) antigen binding site specific for the antigen in the binding molecule.

As used herein, the term “antigen” refers to a molecule, such as a protein, to which an antigen binding molecule binds. Useful antigens can be found, for example, on the surfaces of tumor cells, on the surfaces of tumor stroma cells, on the surfaces of virus-infected cells, on the surfaces of other diseased cells, on the surface of immune cells, free in blood serum, and/or in the extracellular matrix (ECM). In particular aspects, the antigen is a human protein.

As used herein, the term “epitope” refers to a site on an antigen to which an antigen-binding site binds. Epitopes can be formed from a contiguous stretch of amino acids or a conformational configuration made up of different regions of noncontiguous amino acids. Epitopes often include chemically active surface groupings of antigens such as amino acids, glycan side chains, phosphoryl, or sulfonyl, and may have specific three dimensional structural characteristics, and/or specific charge characteristics. Two distinct antigen-binding domains capable of binding the same antigen may bind different epitopes of said antigen. Such distinct antigen-binding domains are said to not compete for binding if both antigen-binding domains can simultaneously bind to the antigen. In this case two distinct antigen binding domains are non-competing. Alternatively, antigen-binding domains may compete for binding, i.e. show competitive binding. This may be due to partially overlapping epitopes or simultaneous binding of the two antigen binding domains is hampered due to steric hindrance. Assays to determine whether antigen-binding domains show non-competitive or competitive binding are well known in the art, for example competitive binding analysis using ELISA, RIA, surface plasmon resonance, flow cytometry or any other quantitative or qualitative antibody-binding assay available in the art.

A “target antigen” as used herein refers to an antigen presented on the surface of a target cell, for example an antigen on a cell in a tumor, such as a cancer cell or a cell of the tumor stroma, or an antigen on a T cell.

A “tumor-associated antigen” as used herein refers to any antigen presented on the surface of a cell in a tumor such as a cancer cell or a cell of the tumor stroma. Particular tumor-associated antigens are CEA, FAP, Her2, Her3 or EGFR.

The term “Fibroblast activation protein (FAP)”, also known as Prolyl endopeptidase FAP or Seprase (EC 3.4.21), refers to any native FAP from any vertebrate source, including mammals such as primates (e.g. humans) non-human primates (e.g. cynomolgus monkeys) and rodents (e.g. mice and rats), unless otherwise indicated. FAP is expressed on Cancer-associated fibroblasts (CAFs) in the tumor stroma. The term encompasses “full-length,” unprocessed FAP as well as any form of FAP which results from processing in the cell. The term also encompasses naturally occurring variants of FAP, e.g., splice variants or allelic variants. In one embodiment, the antigen binding molecule of the invention is capable of specific binding to human, mouse and/or cynomolgus FAP. The amino acid sequence of human FAP is shown in UniProt (www.uniprot.org) accession no. Q12884 (version 149), or NCBI (www.ncbi.nlm.nih.gov/) RefSeq NP 004451.2. The extracellular domain (ECD) of human FAP extends from amino acid position 26 to 760. The amino acid sequence of mouse FAP is shown in UniProt accession no. P97321 (version 126), or NCBI RefSeq NP_032012.1. The extracellular domain (ECD) of mouse FAP extends from amino acid position 26 to 761. Preferably, an anti-FAP binding molecule binds to the extracellular domain of FAP. Exemplary anti-FAP binding molecules are described in International Patent Application No. WO 2012/020006 A2 and WO 2020/070041 A1.

The term “Carcinoembroynic antigen (CEA)”, also known as Carcinoembryonic antigen-related cell adhesion molecule 5 (CEACAM5), refers to any native CEA from any vertebrate source, including mammals such as primates (e.g. humans) non-human primates (e.g. cynomolgus monkeys) and rodents (e.g. mice and rats), unless otherwise indicated. The amino acid sequence of human CEA is shown in UniProt accession no. P06731 (version 151). CEA has long been identified as a tumor-associated antigen (Gold and Freedman, J Exp Med., 121:439-462, 1965; Berinstein N. L., J Clin Oncol., 20:2197-2207, 2002). Originally classified as a protein expressed only in fetal tissue, CEA has now been identified in several normal adult tissues. These tissues are primarily epithelial in origin, including cells of the gastrointestinal, respiratory, and urogential tracts, and cells of colon, cervix, sweat glands, and prostate (Nap et al., Tumour Biol., 9(2-3):145-53, 1988; Nap et al., Cancer Res., 52(8):2329-23339, 1992). Tumors of epithelial origin, as well as their metastases, contain CEA as a tumor associated antigen. While the presence of CEA itself does not indicate transformation to a cancerous cell, the distribution of CEA is indicative. In normal tissue, CEA is generally expressed on the apical surface of the cell (Hammarstrom S., Semin Cancer Biol. 9(2):67-81 (1999)), making it inaccessible to antibody in the blood stream. In contrast to normal tissue, CEA tends to be expressed over the entire surface of cancerous cells (Hammarstrom S., Semin Cancer Biol. 9(2):67-81 (1999)). This change of expression pattern makes CEA accessible to antibody binding in cancerous cells. In addition, CEA expression increases in cancerous cells. Furthermore, increased CEA expression promotes increased intercellular adhesions, which may lead to metastasis (Marshall J., Semin Oncol., 30 (a Suppl. 8):30-6, 2003). The prevalence of CEA expression in various tumor entities is generally very high. In concordance with published data, own analyses performed in tissue samples confirmed its high prevalence, with approximately 95% in colorectal carcinoma (CRC), 90% in pancreatic cancer, 80% in gastric cancer, 60% in non-small cell lung cancer (NSCLC, where it is co-expressed with HER3), and 40% in breast cancer; low expression was found in small cell lung cancer and glioblastoma.

“HER2” (also known as erbB-2 or CD340) refers to any native HER2 from any vertebrate source, including mammals such as primates (e.g. humans), non-human primates (e.g. cynomolgus monkeys) and rodents (e.g. mice and rats), unless otherwise indicated. The term encompasses “full-length,” unprocessed HER2 as well as any form of HER2 that results from processing in the cell. The term also encompasses naturally occurring variants of HER2, e.g., splice variants or allelic variants. In one aspect, HER2 is human HER2. The amino acid sequence of human HER2 is shown in UniProt (www.uniprot.org) entry no. Q9UK79 (version 95).

“Epidermal Growth Factor Receptor (EGFR)”, also named Proto-oncogene c-ErbB-1 or Receptor tyrosine-protein kinase erbB-1, refers to any native EGFR from any vertebrate source, including mammals such as primates (e.g. humans) non-human primates (e.g. cynomolgus monkeys) and rodents (e.g. mice and rats), unless otherwise indicated. The amino acid sequence of human EGFR is shown in UniProt accession no. P00533.

A “T cell antigen” as used herein refers to any antigen present on the surface of a T lymphocyte.

The term “PD-1”, also known as CD279, PD1 or programmed cell death protein 1, refers to any native PD-1 from any vertebrate source, including mammals such as primates (e.g. humans) non-human primates (e.g. cynomolgus monkeys) and rodents (e.g. mice and rats), in particular to the human protein PD-1 with the amino acid sequence as shown in UniProt (www.uniprot.org) accession no. Q15116.

The term “interferon gamma” or “IFNγ” or “IFN-γ” as used herein, refers to any native IFNγ from any vertebrate source, including mammals such as primates (e.g. humans) and rodents (e.g., mice and rats), unless otherwise indicated. The term encompasses unprocessed IFNγ as well as any form of IFNγ that results from processing in the cell. The term also encompasses naturally occurring variants of IFNγ, e.g. splice variants or allelic variants.

The term “interleukin-2” or “IL-2” as used herein, refers to any native IL-2 from any vertebrate source, including mammals such as primates (e.g. humans) and rodents (e.g., mice and rats), unless otherwise indicated. The term encompasses unprocessed IL-2 as well as any form of IL-2 that results from processing in the cell. The term also encompasses naturally occurring variants of IL-2, e.g. splice variants or allelic variants.

Cytokine receptors are cell-surface glycoproteins that specifically bind cytokines and transduce their signals. Generally, cytokine receptors function as oligomeric complexes consisting of typically two to four receptor chains, also termed subunits, that may be the same or different. Thus, the term “cytokine receptor complex” refers to cytokine receptors composed of at least two subunits.

The IFNγ receptor complex comprises the IFNγR1 subunit and the IFNγR2 subunit. The term “Interferon gamma receptor 1” or “IFNγR1”, also referred to as CD119 (Cluster of differentiation 119) or Interferon gamma receptor α-chain (IFNγRα), refers to any native IFNγR1 from any vertebrate source, including mammals such as primates (e.g. humans) and rodents (e.g., mice and rats), unless otherwise indicated. The term encompasses “full-length”, unprocessed IFNγR1 as well as any form of IFNγR1 that results from processing in the cell. The term also encompasses naturally occurring variants of IFNγR1, e.g. splice variants or allelic variants. In certain embodiments IFNγR1 is human IFNγR1.

The term “Interferon gamma receptor 2” or “IFNγR2”, also referred to as Interferon gamma receptor β-chain (IFNγRβ), refers to any native IFNγR2 from any vertebrate source, including mammals such as primates (e.g. humans) and rodents (e.g., mice and rats), unless otherwise indicated. The term encompasses “full-length”, unprocessed IFNγR2 as well as any form of IFNγR2 that results from processing in the cell. The term also encompasses naturally occurring variants of IFNγR2, e.g. splice variants or allelic variants. In certain embodiments IFNγR2 is human IFNγR2.

The term “IL-2Rα” or “α-subunit of the IL-2 receptor” also known as CD25, as used herein, refers to any native IL-2Rα from any vertebrate source, including mammals such as primates (e.g. humans) and rodents (e.g., mice and rats), unless otherwise indicated. The term encompasses “full-length”, unprocessed IL-2Rα as well as any form of IL-2Rα that results from processing in the cell. The term also encompasses naturally occurring variants of IL-2Rα, e.g. splice variants or allelic variants. In certain embodiments IL-2Rα is human IL-2Rα.

The term “IL-2Rβ” or “β-subunit of the IL-2 receptor”, also known as CD122 or p70, refers to any native IL-2Rβ from any vertebrate source, including mammals such as primates (e.g. humans) and rodents (e.g., mice and rats), unless otherwise indicated. The term encompasses “full-length”, unprocessed IL-2Rβ as well as any form of IL-2Rβ that results from processing in the cell. The term also encompasses naturally occurring variants of IL-2Rβ, e.g. splice variants or allelic variants. In certain embodiments IL-2Rβ is human IL-2Rβ.

The term “IL-2Rγ” or “γ-subunit of the IL-2 receptor”, also known as common cytokine receptor γ-subunit, common γ-chain, γc, or CD132, refers to any native IL-2Rγ from any vertebrate source, including mammals such as primates (e.g. humans) and rodents (e.g., mice and rats), unless otherwise indicated. The term encompasses “full-length”, unprocessed IL-2Rγ as well as any form of IL-2Rγ that results from processing in the cell. The term also encompasses naturally occurring variants of IL-2Rγ, e.g. splice variants or allelic variants. In certain embodiments IL-2Rγ is human IL-2Rγ.

Different associations of the individual IL-2R subunits IL-2Rα, IL-2Rβ and IL-2Rγ, can produce three IL-2R forms that differ in their affinity to IL-2. The high-affinity IL-2R refers to the heterotrimeric form of the IL-2R, consisting of IL-2Rα, IL-2Rβ and IL-2Rγ. The intermediate-affinity IL-2R refers to the heterodimeric form of the IL-2R, consisting of IL-2Rβ and IL-2Rγ. Whereas, the low affinity IL-2R refers to the monomeric form of the IL-2R, consisting solely of IL-2Rα (for a review see e.g. Olejniczak and Kasprzak, Med Sci Monit 14, RA179-189 (2008)).

The term “IL-2 receptor complex” as used herein refers to the high-affinity IL-2 receptor or the intermediate-affinity IL-2 receptor.

The term “Fc domain” or “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain.

Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, numbering according to Kabat EU index). Therefore, the C-terminal lysine (Lys447), or the C-terminal glycine (Gly446) and lysine (Lys447), of the Fc region may or may not be present. In one aspect, a heavy chain including an Fc region (subunit) as specified herein, comprised in a binding molecule according to the invention, comprises an additional C-terminal lysine (K447, numbering according to Kabat EU index). In one aspect, a heavy chain including an Fc region (subunit) as specified herein, comprised in a binding molecule according to the invention, comprises a C-terminal glycine residue (G446, numbering according to Kabat EU index) and does not comprise a C-terminal lysine (Lys477). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991 (see also above). A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein preferably includes separate modifications made to each of the two Fc domain subunits desired to associate (i.e. the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which may be non-identical in the sense that further components fused to each of the subunits (e.g. antigen binding domains) are not the same. In one aspect, the modification promoting the association of the first and the second subunit of the Fc domain comprises an amino acid mutation in the Fc domain, specifically an amino acid substitution. In particular aspects, the modification promoting the association of the first and the second subunit of the Fc domain comprises a separate amino acid mutation, specifically an amino acid substitution, in each of the two subunits of the Fc domain.

One heterodimerization approach known in the art is the so-called “knobs-into-holes” technology, which is described in detail providing several examples in e.g. WO 96/027011, Ridgway, J. B., et al., Protein Eng. 9 (1996) 617-621; Merchant, A. M., et al., Nat. Biotechnol. 16 (1998) 677-681; and WO98/050431. In the “knobs-into-holes” technology, within the interface formed between two CH3 domains in the tertiary structure of the antibody, particular amino acids on each CH3 domain are engineered to produce a protuberance (“knob”) in one of the CH3 domains and a cavity (“hole”) in the other one of the CH3 domains, respectively. In the tertiary structure of the multispecific antibody the introduced protuberance in the one CH3 domain is positionable in the introduced cavity in the other CH3 domain.

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B-cell receptor), and B-cell activation.

An “activating Fc receptor” is an Fc receptor that following engagement by an Fc domain of an antibody elicits signaling events that stimulate the receptor-bearing cell to perform effector functions. Human activating Fc receptors include FcγRIIIa (CD16a), FcγRI (CD64), FcγRIIa (CD32), and FcαRI (CD89).

Antibody-dependent cell-mediated cytotoxicity (ADCC) is an immune mechanism leading to the lysis of antibody-coated target cells by immune effector cells. The target cells are cells to which antibodies or derivatives thereof comprising an Fc region specifically bind, generally via the protein part that is N-terminal to the Fc region. As used herein, the term “reduced ADCC” is defined as either a reduction in the number of target cells that are lysed in a given time, at a given concentration of antibody in the medium surrounding the target cells, by the mechanism of ADCC defined above, and/or an increase in the concentration of antibody in the medium surrounding the target cells, required to achieve the lysis of a given number of target cells in a given time, by the mechanism of ADCC. The reduction in ADCC is relative to the ADCC mediated by the same antibody produced by the same type of host cells, using the same standard production, purification, formulation and storage methods (which are known to those skilled in the art), but that has not been engineered. For example, the reduction in ADCC mediated by an antibody comprising in its Fc domain an amino acid substitution that reduces ADCC, is relative to the ADCC mediated by the same antibody without this amino acid substitution in the Fc domain. Suitable assays to measure ADCC are well known in the art (see e.g. PCT publication no. WO 2006/082515 or PCT publication no. WO 2012/130831).

“Reduced binding”, for example reduced binding to an Fc receptor, refers to a decrease in affinity for the respective interaction, as measured for example by SPR. For clarity, the term includes also reduction of the affinity to zero (or below the detection limit of the analytic method), i.e. complete abolishment of the interaction. Conversely, “increased binding” refers to an increase in binding affinity for the respective interaction.

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., an antibody and an antigen). The affinity of a molecule X for its partner Y can generally be represented by the dissociation constant (KD). Affinity can be measured by well-established methods known in the art, including those described herein. A preferred method for measuring affinity is Surface Plasmon Resonance (SPR).

As used herein, the terms “engineer, engineered, engineering”, are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches.

The term “amino acid mutation” as used herein is meant to encompass amino acid substitutions, deletions, insertions, and modifications. Any combination of substitution, deletion, insertion, and modification can be made to arrive at the final construct, provided that the final construct possesses the desired characteristics, e.g., reduced binding to an Fc receptor, or increased association with another peptide. Amino acid sequence deletions and insertions include amino- and/or carboxy-terminal deletions and insertions of amino acids. Preferred amino acid mutations are amino acid substitutions. For the purpose of altering e.g. the binding characteristics of an Fc region, nonconservative amino acid substitutions, i.e. replacing one amino acid with another amino acid having different structural and/or chemical properties, are particularly preferred. Amino acid substitutions include replacement by non-naturally occurring amino acids or by naturally occurring amino acid derivatives of the twenty standard amino acids (e.g. 4-hydroxyproline, 3-methylhistidine, ornithine, homoserine, 5-hydroxylysine). Amino acid mutations can be generated using genetic or chemical methods well known in the art. Genetic methods may include site directed mutagenesis, PCR, gene synthesis and the like. It is contemplated that methods of altering the side chain group of an amino acid by methods other than genetic engineering, such as chemical modification, may also be useful. Various designations may be used herein to indicate the same amino acid mutation. For example, a substitution from proline at position 329 of the Fc domain to glycine can be indicated as 329G, G329, G329, P329G, or Pro329Gly.

By “fused” is meant that the components (e.g. a Fab molecule and an Fc domain subunit) are linked by peptide bonds, either directly or via one or more peptide linkers. The term “fusion point” refers to the site on one component at which said component is fused to another component. For example a cytokine receptor-binding domain may be fused at its C-terminus to the N-terminus of the VH domain of a target-binding domain. Thus, the fusion point on the cytokine receptor-binding domain is the C-terminus and the fusion point on the target-binding domain is the N-terminus.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

Unless otherwise indicated, for purposes herein, % amino acid sequence identity values are generated using the ggsearch program of the FASTA package version 36.3.8c or later with a BLOSUM50 comparison matrix. The FASTA program package was authored by W. R. Pearson and D. J. Lipman (“Improved Tools for Biological Sequence Analysis”, PNAS 85 (1988) 2444-2448), W. R. Pearson (“Effective protein sequence comparison” Meth. Enzymol. 266 (1996) 227-25 258), and Pearson et. al. (Genomics 46 (1997) 24-36) and is publicly available from www.fasta.bioch.virginia.edu/fasta_www2/fasta_down.shtml or www.ebi.ac.uk/Tools/sss/fasta.

Alternatively, a public server accessible at fasta.bioch.virginia.edu/fasta_www2/index.cgi can be used to compare the sequences, using the ggsearch (global protein:protein) program and default options (BLOSUM50; open: −10; ext: −2; Ktup=2) to ensure a global, rather than local, alignment is performed. Percent amino acid identity is given in the output alignment header.

### Cytokine Mimetics

The present inventors have found that a pair of antigen binding domains capable of binding different cytokine receptor subunits of a cytokine receptor complex, hereinafter referred to as cytokine receptor-binding domains, can act as cytokine agonists, i.e. mimic a naturally occurring cytokine. Cytokine agonists are also termed cytokine mimetics. This has been shown by combining a pair of cytokine receptor-binding domains capable of binding different cytokine receptor subunits in a single antigen binding molecule. The antibody format of these molecules may provide advantageous properties compared to natural or recombinant cytokines. To achieve conditional activation of a cytokine receptor, the pair of cytokine receptor-binding domains were split in two distinct antigen binding molecules, i.e. split molecules or split antibodies. These antigen binding molecules further comprise an antigen binding domain capable of binding a target antigen, hereinafter referred to as target-binding domain. By target-dependent assembly of the pair of cytokine receptor-binding domains, the molecules can act as cytokine mimetics. Using a target-dependent approach further allows to direct the agonistic activity to a site of interest, such as cells or tissues expressing the target. The target-binding domains of the molecules are capable of binding the same target antigen simultaneously, i.e. they bind different epitopes of said antigen and are non-competing. Such a pair of target-binding domains are also referred to as biparatopic target-binding domains. The assembly of the cytokine receptor-binding domains by the biparatopic target-binding domains allows to target the cytokine receptor-binding domains specifically to a site of interest, i.e. a cell or environment where the target antigen is expresses, to act as cytokine mimetics. The pair of split antigen binding molecules are thus a biparatopic pair of antigen binding molecules.

### Split Biparatopic Antigen Binding Molecule Pairs

In one aspect, the present invention provides a pair of antigen binding molecules that specifically bind to a target antigen, comprising a) first antigen binding molecule comprising i) a first target-binding domain, ii) a first cytokine receptor-binding domain and iii) a Fc domain; and a second antigen binding molecule comprising i) a second target-binding domain, ii) a second cytokine receptor-binding domain and iii) a Fc domain; wherein the first target-binding domain is capable of binding a first epitope of the target antigen and the second target-binding domain is capable of binding a second epitope of the target antigen, wherein the first and the second target-binding domains do not compete for binding on the target antigen; and wherein the first cytokine receptor-binding domain is capable of binding a first cytokine receptor subunit and the second cytokine receptor-binding domain is capable of binding a second cytokine receptor subunit.

The pair of antigen binding molecules described above and herein, may incorporate, singly or in combination, any of the features described in the following (unless the context dictates otherwise).

### Cytokine Receptor-Binding Domain

According to the invention, each antigen binding molecule of the pair of antigen binding molecules comprises a cytokine receptor-binding domain. The first antigen binding molecule comprises a first cytokine receptor-binding domain and the second antigen binding molecule comprises a second cytokine receptor-binding domain. The cytokine receptor-binding domains comprised in the pair of antigen binding molecules bind different cytokine receptor subunits. Each cytokine receptor-binding domain of the pair of antigen binding molecules binds a distinct cytokine receptor subunit of a cytokine receptor complex. Thus, the first antigen binding molecule comprises a first cytokine receptor-binding domain capable of binding a first cytokine receptor subunit and the second antigen binding molecule comprises a second cytokine receptor-binding domain capable of binding a second cytokine receptor subunit. The first and the second cytokine receptor subunit are subunits of a cytokine receptor complex.

The cytokine receptor complex may be IFNγ receptor complex, IL-2 receptor complex, IL-7 receptor complex, IL-12 receptor complex or IL-18 receptor complex. Thus, the first and the second cytokine receptor-binding domain may bind different subunits of the IFNγ receptor complex, IL-2 receptor complex, IL-7 receptor complex, IL-12 receptor complex or IL-18 receptor complex. In one embodiment, the first and second cytokine receptor-binding domains bind subunits of the IFNγ receptor complex. Thus, in one embodiment the first cytokine receptor-binding domain is capable of binding IFNγR1 and the second cytokine receptor-binding domain is capable of binding IFNγR2. Alternatively, the first cytokine receptor-binding domain is capable of binding IFNγR2 and the second cytokine receptor-binding domain is capable of binding IFNγR1. In a further embodiment, the first and second cytokine receptor-binding domains bind subunits of the IL-2 receptor complex. Thus, in one embodiment the first cytokine receptor-binding domain is capable of binding IL-2Rβ and the second cytokine receptor-binding domain is capable of binding IL-2Rγ. In one embodiment, the first cytokine receptor-binding domain is capable of binding IL-2Rγ and the second cytokine receptor-binding domain is capable of binding IL-2Rβ. In another embodiment, the first and second cytokine receptor-binding domains bind subunits of the IL-7 receptor complex. Thus, in one embodiment the first cytokine receptor-binding domain is capable of binding IL-7Rα and the second cytokine receptor-binding domain is capable of binding IL-2Rγ. In one embodiment, the first cytokine receptor-binding domain is capable of binding IL-2Rγ and the second cytokine receptor-binding domain is capable of binding IL-7Rα. In a further embodiment, the first and second cytokine receptor-binding domains bind subunits of the IL-12 receptor complex. Thus, in one embodiment the first cytokine receptor-binding domain is capable of binding IL-12Rβ1 and the second cytokine receptor-binding domain is capable of binding IL-12Rβ2. In one embodiment, the first cytokine receptor-binding domain is capable of binding IL-12Rβ2 and the second cytokine receptor-binding domain is capable of binding IL-12Rβ1.

The first and/or second cytokine receptor-binding domain may be an antibody fragment, such as Fv, Fab, scFv, scFab or single-domain antibody. In one embodiment, the first and/or second cytokine receptor-binding domains are single-domain antibodies. In a particular embodiment, the first and second cytokine receptor-binding domains are VHH domains.

In one aspect, the first cytokine receptor-binding domain comprises an amino acid sequence selected from SEQ ID NO: 1, SEQ ID NO: 2, SEQ ID NO: 3, SEQ ID NO: 4 and SEQ ID NO: 5 and the second cytokine receptor-binding domain comprises an amino acid sequence selected from SEQ ID NO: 6, SEQ ID NO: 7, SEQ ID NO: 8, SEQ ID NO: 9 and SEQ ID NO: 10. In one aspect, the first cytokine receptor-binding domain comprises an amino acid sequence selected from SEQ ID NO: 6, SEQ ID NO: 7, SEQ ID NO: 8, SEQ ID NO: 9 and SEQ ID NO: 10, and the second cytokine receptor-binding domain comprises an amino acid sequence selected from SEQ ID NO: 1, SEQ ID NO: 2, SEQ ID NO: 3, SEQ ID NO: 4 and SEQ ID NO: 5. In one aspect, the first cytokine receptor-binding domain comprises the sequence of SEQ ID NO: 3 and the second cytokine receptor-binding domain comprises the sequence of SEQ ID NO: 7. In one aspect, the first cytokine receptor-binding domain comprises the sequence of SEQ ID NO: 7 and the second cytokine receptor-binding domain comprises the sequence of SEQ ID NO: 3.

In one aspect, the first cytokine receptor-binding domain comprises an amino acid sequence selected from SEQ ID NO: 56, SEQ ID NO: 57, SEQ ID NO: 58, SEQ ID NO: 59, SEQ ID NO: 60, and the second cytokine receptor-binding domain comprises an amino acid sequence selected from SEQ ID NO: 61, SEQ ID NO: 62, SEQ ID NO: 63, SEQ ID NO: 64, SEQ ID NO: 65. In one aspect, the first cytokine receptor-binding domain comprises an amino acid sequence selected from SEQ ID NO: 61, SEQ ID NO: 62, SEQ ID NO: 63, SEQ ID NO: 64, SEQ ID NO: 65, and the second cytokine receptor-binding domain comprises an amino acid sequence selected from SEQ ID NO: 56, SEQ ID NO: 57, SEQ ID NO: 58, SEQ ID NO: 59, SEQ ID NO: 60. In one aspect, the first cytokine receptor-binding domain comprises the sequence of SEQ ID NO: 60 and the second cytokine receptor-binding domain comprises the sequence of SEQ ID NO: 62. In one aspect, the first cytokine receptor-binding domain comprises the sequence of SEQ ID NO: 62 and the second cytokine receptor-binding domain comprises the sequence of SEQ ID NO: 60.

In one aspect, the first cytokine receptor-binding domain comprises an amino acid sequence selected from SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, and the second cytokine receptor-binding domain comprises an amino acid sequence selected from SEQ ID NO: 61, SEQ ID NO: 62, SEQ ID NO: 63, SEQ ID NO: 64, SEQ ID NO: 65. In one aspect, the first cytokine receptor-binding domain comprises an amino acid sequence selected from SEQ ID NO: 61, SEQ ID NO: 62, SEQ ID NO: 63, SEQ ID NO: 64, SEQ ID NO: 65, and the second cytokine receptor-binding domain comprises an amino acid sequence selected from SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192. In one aspect, the first cytokine receptor-binding domain comprises an amino acid sequence selected from SEQ ID NO: 177, SEQ ID NO: 178, SEQ ID NO: 179, SEQ ID NO: 181, SEQ ID NO: 182, SEQ ID NO: 183, SEQ ID NO: 186, SEQ ID NO: 187, SEQ ID NO: 189, SEQ ID NO: 190, SEQ ID NO: 191, SEQ ID NO: 192, and the second cytokine receptor-binding domain comprises the sequence of SEQ ID NO: 62. In one aspect, the first cytokine receptor-binding domain comprises the sequence of SEQ ID NO: 62 and the second cytokine receptor-binding domain comprises an amino acid sequence selected from SEQ ID NO: 177, SEQ ID NO: 178, SEQ ID NO: 179, SEQ ID NO: 181, SEQ ID NO: 182, SEQ ID NO: 183, SEQ ID NO: 186, SEQ ID NO: 187, SEQ ID NO: 189, SEQ ID NO: 190, SEQ ID NO: 191, SEQ ID NO: 192. In one aspect, the first cytokine receptor-binding domain comprises the sequence of SEQ ID NO: 182 and the second cytokine receptor-binding domain comprises the sequence of SEQ ID NO: 62. In one aspect, the first cytokine receptor-binding domain comprises the sequence of SEQ ID NO: 62 and the second cytokine receptor-binding domain comprises the sequence of SEQ ID NO: 182. In one aspect, the first cytokine receptor-binding domain comprises the sequence of SEQ ID NO: 186 and the second cytokine receptor-binding domain comprises the sequence of SEQ ID NO: 62. In one aspect, the first cytokine receptor-binding domain comprises the sequence of SEQ ID NO: 62 and the second cytokine receptor-binding domain comprises the sequence of SEQ ID NO: 186. In one aspect, the first cytokine receptor-binding domain comprises the sequence of SEQ ID NO: 190 and the second cytokine receptor-binding domain comprises the sequence of SEQ ID NO: 62. In one aspect, the first cytokine receptor-binding domain comprises the sequence of SEQ ID NO: 62 and the second cytokine receptor-binding domain comprises the sequence of SEQ ID NO: 190.

### Target Antigen-Binding Domain

According to the invention, each antigen binding molecule of the pair of antigen binding molecules comprises a target-binding domain. The first antigen binding molecule comprises a first target-binding domain and the second antigen binding molecule comprises a second target-binding domain. Both the first and the second target-binding domain bind the same target, i.e. the same antigen. Yet, the first and the second target-binding domains bind distinct epitopes of said target. The first target-binding domain is capable of binding a first epitope on the target antigen and the second target-binding domain is capable of binding a second epitope on the target antigen. The first and the second target-binding domain are capable of binding the target simultaneously, i.e. the target-binding domains are non-competing domains. The first target-binding domain and the second target-binding domain do not compete for binding on the target antigen.

The target-binding domain of the first and/or second antigen binding molecule may be an antibody fragment. In one embodiment, the first target-binding domain is an antibody fragment and the second target-binding domain is an antibody fragment. In one embodiment, the first antigen binding molecule comprises a first target-binding domain, wherein the target binding domain is an antibody fragment, and the second antigen binding molecule comprises a second target-binding domain, wherein the target-binding domain is an antibody fragment. The target-binding domain of the first and/or second antigen binding molecule may be a Fv, Fab, scFv, scFab molecule or a single domain antibody. In one embodiment, the first antigen binding molecule comprises a first target-binding domain, wherein the target binding domain is a Fv, Fab, scFv, scFab molecule or a single domain antibody, and the second antigen binding molecule comprises a second target-binding domain, wherein the target-binding domain is a Fv, Fab, scFv, scFab molecule or a single domain antibody.

In one embodiment, the target-binding domain of the first and/or second antigen binding molecule is a Fab molecule. In one embodiment, the first target-binding domain is a Fab molecule and the second target-binding domain is a Fab molecule. In one embodiment, the first antigen binding molecule comprises a first target-binding domain, wherein the target binding domain is a Fab molecule, and the second antigen binding molecule comprises a second target-binding domain, wherein the target-binding domain is a Fab molecule. In one embodiment, the first target-binding domain comprises a heavy chain variable domain (VH₁), a light chain variable domain (VL₁), a heavy chain constant domain (CH1₁) and a light chain constant domain (CL₁). In one embodiment, the second target-binding domain comprises a heavy chain variable domain (VH₂), a light chain variable domain (VL₂), a heavy chain constant domain (CH1₂) and a light chain constant domain (CL₂).

The target-binding domain may be a cross-Fab. In one embodiment, the first target-binding domain is a cross-Fab. In another embodiment, the second target-binding domain is a cross-Fab. In one embodiment, the first antigen binding molecule comprises a first target-binding domain, wherein the target-binding domain is a Fab molecule, wherein the Fab molecule is a cross-Fab, and the second antigen binding molecule comprises a second target-binding domain, wherein the target-binding domain is a Fab molecule, wherein the Fab molecule is in not a cross-Fab, i.e. a conventional Fab molecule. In one embodiment, the first antigen binding molecule comprises a first target-binding domain, wherein the target-binding domain is Fab molecule, wherein the Fab molecule is a conventional Fab molecule, and the second antigen binding molecule comprises a second target-binding domain, wherein the target-binding domain is a Fab molecule, wherein the Fab molecule is a cross-Fab.

Both the first and the second target-binding domains specifically bind the same target antigen. The target antigen may be a tumor-associated antigen or a T cell antigen.

The target antigen may be a tumor-associated antigen such as CEA, FAP, Her2, Her3 or EGFR. The target antigen may be a human tumor-associated antigen. Thus, the target antigen may be human CEA, human FAP, human Her2, human Her3 or human EGFR. Thus, in one aspect the first target-binding domain is capable of binding a first epitope of CEA and the second target-binding domain is capable of binding a second epitope of CEA. In one aspect the first target-binding domain is capable of binding a first epitope of FAP and the second target-binding domain is capable of binding a second epitope of FAP. In one aspect the first target-binding domain is capable of binding a first epitope of Her2 and the second target-binding domain is capable of binding a second epitope of Her2. In one aspect the first target-binding domain is capable of binding a first epitope of Her3 and the second target-binding domain is capable of binding a second epitope of Her3. In one aspect the first target-binding domain is capable of binding a first epitope of EGFR and the second target-binding domain is capable of binding a second epitope of EGFR.

In one aspect, the first target-binding domain comprises a VH₁ of SEQ ID NO: 20 and a VL₁ of SEQ ID NO: 21 and the second target-binding domain comprises a VH₂ of SEQ ID NO: 22 and a VL₂ of SEQ ID NO: 23. In one aspect, the first target-binding domain comprises a VH₁ of SEQ ID NO: 22 and a VL₁ of SEQ ID NO: 23 and the second target-binding domains comprises a VH₂ of SEQ ID NO: 20 and a VL₂ of SEQ ID NO: 21. In one aspect, the first target-binding domain comprises a VH₁ of SEQ ID NO: 114 and a VL₁ of SEQ ID NO: 115 and the second target-binding domain comprises a VH₂ of SEQ ID NO: 116 and a VL₂ of SEQ ID NO: 117. In one aspect, the first target-binding domain comprises a VH₁ of SEQ ID NO: 116 and a VL₁ of SEQ ID NO: 117 and the second target-binding domains comprises a VH₂ of SEQ ID NO: 114 and a VL₂ of SEQ ID NO: 115. In one aspect, the first target-binding domain comprises a VH₁ of SEQ ID NO: 130 and a VL₁ of SEQ ID NO: 131 and the second target-binding domain comprises a VH₂ of SEQ ID NO: 132 and a VL₂ of SEQ ID NO: 133. In one aspect, the first target-binding domain comprises a VH₁ of SEQ ID NO: 132 and a VL₁ of SEQ ID NO: 133 and the second target-binding domains comprises a VH₂ of SEQ ID NO: 130 and a VL₂ of SEQ ID NO: 131. In one aspect, the first target-binding domain comprises a VH₁ of SEQ ID NO: 140 and a VL₁ of SEQ ID NO: 141 and the second target-binding domain comprises a VH₂ of SEQ ID NO: 142 and a VL₂ of SEQ ID NO: 143. In one aspect, the first target-binding domain comprises a VH₁ of SEQ ID NO: 142 and a VL₁ of SEQ ID NO: 143 and the second target-binding domains comprises a VH₂ of SEQ ID NO: 140 and a VL₂ of SEQ ID NO: 141.

The target antigen may be a T cell antigen such as PD-1 or LAG-3. The target antigen may be human PD-1 or human LAG-3. Thus, in one aspect the first target-binding domain is capable of binding a first epitope of PD-1 and the second target-binding domain is capable of binding a second epitope of PD-1. In one aspect the first target-binding domain is capable of binding a first epitope of LAG-3 and the second target-binding domain is capable of binding a second epitope of LAG-3. In one aspect, the first target-binding domain comprises a VH₁ of SEQ ID NO: 76 and a VL₁ of SEQ ID NO: 77 and the second target-binding domain comprises a VH₂ of SEQ ID NO: 78 and a VL₂ of SEQ ID NO: 79. In one aspect, the first target-binding domain comprises a VH₁ of SEQ ID NO: 78 and a VL₁ of SEQ ID NO: 79 and the second target-binding domains comprises a VH₂ of SEQ ID NO: 76 and a VL₂ of SEQ ID NO: 77. In one aspect, the first target-binding domain comprises a VH₁ of SEQ ID NO: 93 and a VL₁ of SEQ ID NO: 94 and the second target-binding domain comprises a VH₂ of SEQ ID NO: 78 and a VL₂ of SEQ ID NO: 79. In one aspect, the first target-binding domain comprises a VH₁ of SEQ ID NO: 78 and a VL₁ of SEQ ID NO: 79 and the second target-binding domains comprises a VH₂ of SEQ ID NO: 93 and a VL₂ of SEQ ID NO: 94. In one aspect, the first target-binding domain comprises a VH₁ of SEQ ID NO: 150 and a VL₁ of SEQ ID NO: 151 and the second target-binding domain comprises a VH₂ of SEQ ID NO: 152 and a VL₂ of SEQ ID NO: 153. In one aspect, the first target-binding domain comprises a VH₁ of SEQ ID NO: 152 and a VL₁ of SEQ ID NO: 153 and the second target-binding domains comprises a VH₂ of SEQ ID NO: 150 and a VL₂ of SEQ ID NO: 151.

### Fc Domain

According to the invention, the first and the second antigen binding molecules comprise an Fc domain.

The Fc domain of the binding molecule(s) consists of a pair of polypeptide chains, a first Fc domain subunit and a second Fc domain subunit. The first and second Fc domain subunits may comprise heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other.

In one aspect, the first antigen binding molecule comprises a Fc domain composed of a first and a second Fc domain subunit, and/or the second antigen binding molecule comprises a Fc domain composed of a first and a second Fc domain subunit. In one aspect, the first antigen binding molecule comprises a Fc domain composed of a first and a second Fc domain subunit, and the second antigen binding molecule comprises a Fc domain composed of a first and a second Fc domain subunit. In one aspect, each of the antigen binding molecules comprises not more than one Fc domain. one aspect The Fc domain of the first and/or the second antigen binding molecule may be an IgG Fc domain. In a particular embodiment, the Fc domain is an IgG₁ Fc domain. In another particular embodiment, the Fc domain is an IgG₄ Fc domain.

In a more specific aspect, the Fc domain is an IgG₄ Fc domain comprising an amino acid substitution at position S228 (Kabat EU index numbering), particularly the amino acid substitution S228P. This amino acid substitution reduces in vivo Fab arm exchange of IgG₄ antibodies (see Stubenrauch et al., Drug Metabolism and Disposition 38, 84-91 (2010)). In further aspects, the Fc domain is a human Fc domain. In particular aspects, the Fc domain is a human IgG₁ Fc domain.

In one aspect, the Fc domain comprises a modification promoting the association of the first and the second subunit of the Fc domain. Fc domain modifications promoting heterodimerization are further described hereinbelow.

In one aspect, the Fc domain comprises one or more amino acid substitution that reduces binding to an Fc receptor and/or effector function. Fc domain modifications reducing Fc receptor binding and/or effector function are further described hereinbelow.

### Fc Domain Modifications Promoting Heterodimerization

The antigen binding molecule(s) according to the invention comprise a target-binding domain and a cytokine receptor-binding domain, which may be fused to the first and/or the second Fc domain subunit, thus the two Fc domain subunits are typically comprised in two non-identical polypeptide chains. Recombinant co-expression of these polypeptides and subsequent dimerization leads to several possible combinations of the two polypeptides. To improve the yield and purity of the antigen binding molecule(s) in recombinant production, it will thus be advantageous to introduce in the Fc domain of the antigen binding molecule(s) a modification promoting the association of the desired polypeptides.

Accordingly, in particular aspects, the Fc domain of the antigen binding molecule(s) according to the invention comprises a modification promoting the association of the first and the second subunit of the Fc domain. The site of most extensive protein-protein interaction between the two subunits of a human IgG Fc domain is in the CH3 domain of the Fc domain. Thus, in one aspect, said modification is in the CH3 domain of the Fc domain.

There exist several approaches for modifications in the CH3 domain of the Fc domain in order to enforce heterodimerization, which are well described e.g. in WO 96/27011, WO 98/050431, EP 1870459, WO 2007/110205, WO 2007/147901, WO 2009/089004, WO 2010/129304, WO 2011/90754, WO 2011/143545, WO 2012058768, WO 2013157954, WO 2013096291. Typically, in all such approaches the CH3 domain of the first subunit of the Fc domain and the CH3 domain of the second subunit of the Fc domain are both engineered in a complementary manner so that each CH3 domain (or the heavy chain comprising it) can no longer homodimerize with itself but is forced to heterodimerize with the complementarily engineered other CH3 domain (so that the first and second CH3 domain heterodimerize and no homdimers between the two first or the two second CH3 domains are formed).

In specific aspects, said modification promoting the association of the first and the second subunit of the Fc domain is a so-called “knob-into-hole” modification, comprising a “knob” modification in one of the two subunits of the Fc domain and a “hole” modification in the other one of the two subunits of the Fc domain.

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan).

Compensatory cavities of identical or similar size to the protuberances are created in the interface of the second polypeptide by replacing large amino acid side chains with smaller ones (e.g. alanine or threonine).

Accordingly, in preferred aspects, in the CH3 domain of the first subunit of the Fc domain of the antigen binding molecule(s) an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

Preferably said amino acid residue having a larger side chain volume is selected from the group consisting of arginine (R), phenylalanine (F), tyrosine (Y), and tryptophan (W). Preferably said amino acid residue having a smaller side chain volume is selected from the group consisting of alanine (A), serine (S), threonine (T), and valine (V). The protuberance and cavity can be made by altering the nucleic acid encoding the polypeptides, e.g. by site-specific mutagenesis, or by peptide synthesis.

In specific aspects, in (the CH3 domain of) the first subunit of the Fc domain (the “knobs” subunit) the threonine residue at position 366 is replaced with a tryptophan residue (T366W), and in (the CH3 domain of) the second subunit of the Fc domain (the “hole” subunit) the tyrosine residue at position 407 is replaced with a valine residue (Y407V). In one aspect, in the second subunit of the Fc domain additionally the threonine residue at position 366 is replaced with a serine residue (T366S) and the leucine residue at position 368 is replaced with an alanine residue (L368A) (numberings according to Kabat EU index).

In yet further aspects, in the first subunit of the Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second subunit of the Fc domain additionally the tyrosine residue at position 349 is replaced by a cysteine residue (Y349C) (numberings according to Kabat EU index). Introduction of these two cysteine residues results in formation of a disulfide bridge between the two subunits of the Fc domain, further stabilizing the dimer (Carter, J Immunol Methods 248, 7-15 (2001)).

In particular aspects, the first subunit of the Fc domain comprises the amino acid substitutions S354C and T366W, and the second subunit of the Fc domain comprises the amino acid substitutions Y349C, T366S, L368A and Y407V (numbering according to Kabat EU index).

Other techniques of CH3-modification for enforcing the heterodimerization are contemplated as alternatives according to the invention and are described e.g. in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954, WO 2013/096291.

In one aspect, the heterodimerization approach described in EP 1870459, is used alternatively. This approach is based on the introduction of charged amino acids with opposite charges at specific amino acid positions in the CH3/CH3 domain interface between the two subunits of the Fc domain.

A particular aspect for the antigen binding molecule(s) of the invention are amino acid mutations R409D; K370E in one of the two CH3 domains (of the Fc domain) and amino acid mutations D399K; E357K in the other one of the CH3 domains of the Fc domain (numbering according to Kabat EU index).

In one aspect, the antigen binding molecule(s) of the invention comprises amino acid mutation T366W in the CH3 domain of the first subunit of the Fc domain and amino acid mutations T366S, L368A, Y407V in the CH3 domain of the second subunit of the Fc domain, and additionally amino acid mutations R409D; K370E in the CH3 domain of the first subunit of the Fc domain and amino acid mutations D399K; E357K in the CH3 domain of the second subunit of the Fc domain (numberings according to Kabat EU index).

In one aspect, the antigen binding molecule(s) of the invention comprises amino acid mutations S354C, T366W in the CH3 domain of the first subunit of the Fc domain and amino acid mutations Y349C, T366S, L368A, Y407V in the CH3 domain of the second subunit of the Fc domain, or said antigen binding molecule(s) comprises amino acid mutations Y349C, T366W in the CH3 domain of the first subunit of the Fc domain and amino acid mutations S354C, T366S, L368A, Y407V in the CH3 domains of the second subunit of the Fc domain and additionally amino acid mutations R409D; K370E in the CH3 domain of the first subunit of the Fc domain and amino acid mutations D399K; E357K in the CH3 domain of the second subunit of the Fc domain (all numberings according to Kabat EU index).

In one aspect, the heterodimerization approach described in WO 2013/157953 is used alternatively. In one aspect, a first CH3 domain comprises amino acid mutation T366K and a second CH3 domain comprises amino acid mutation L351D (numberings according to Kabat EU index). In further aspects, the first CH3 domain comprises further amino acid mutation L351K. In further aspects, the second CH3 domain comprises further an amino acid mutation selected from Y349E, Y349D and L368E (particularly L368E) (numberings according to Kabat EU index).

In one aspect, the heterodimerization approach described in WO 2012/058768 is used alternatively. In one aspect, a first CH3 domain comprises amino acid mutations L351Y, Y407A and a second CH3 domain comprises amino acid mutations T366A, K409F. In further aspects, the second CH3 domain comprises a further amino acid mutation at position T411, D399, S400, F405, N390, or K392, e.g. selected from a) T411N, T411R, T411Q, T411K, T411D, T411E or T411W, b) D399R, D399W, D399Y or D399K, c) S400E, S400D, S400R, or S400K, d) F405I, F405M, F405T, F405S, F405V or F405W, e) N390R, N390K or N390D, f) K392V, K392M, K392R, K392L, K392F or K392E (numberings according to Kabat EU index). In further aspects, a first CH3 domain comprises amino acid mutations L351Y, Y407A and a second CH3 domain comprises amino acid mutations T366V, K409F. In further aspects, a first CH3 domain comprises amino acid mutation Y407A and a second CH3 domain comprises amino acid mutations T366A, K409F. In further aspects, the second CH3 domain further comprises amino acid mutations K392E, T411E, D399R and S400R (numberings according to Kabat EU index).

In one aspect, the heterodimerization approach described in WO 2011/143545 is used alternatively, e.g. with the amino acid modification at a position selected from the group consisting of 368 and 409 (numbering according to Kabat EU index).

In one aspect, the heterodimerization approach described in WO 2011/090762, which also uses the knobs-into-holes technology described above, is used alternatively. In one aspect, a first CH3 domain comprises amino acid mutation T366W and a second CH3 domain comprises amino acid mutation Y407A. In one aspect, a first CH3 domain comprises amino acid mutation T366Y and a second CH3 domain comprises amino acid mutation Y407T (numberings according to Kabat EU index).

In one aspect, the antigen binding molecule(s) or its Fc domain is of IgG2 subclass and the heterodimerization approach described in WO 2010/129304 is used alternatively.

In an alternative aspect, a modification promoting association of the first and the second subunit of the Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable. In some such aspects, a first CH3 domain comprises amino acid substitution of K392 or N392 with a negatively charged amino acid (e.g. glutamic acid (E), or aspartic acid (D), particularly K392D or N392D) and a second CH3 domain comprises amino acid substitution of D399, E356, D356, or E357 with a positively charged amino acid (e.g. lysine (K) or arginine (R), particularly D399K, E356K, D356K, or E357K, and more particularly D399K and E356K). In further aspects, the first CH3 domain further comprises amino acid substitution of K409 or R409 with a negatively charged amino acid (e.g. glutamic acid (E), or aspartic acid (D), particularly K409D or R409D). In further aspects, the first CH3 domain further or alternatively comprises amino acid substitution of K439 and/or K370 with a negatively charged amino acid (e.g. glutamic acid (E), or aspartic acid (D)) (all numberings according to Kabat EU index).

In one aspect, the heterodimerization approach described in WO 2007/147901 is used alternatively. In one aspect, a first CH3 domain comprises amino acid mutations K253E, D282K, and K322D and a second CH3 domain comprises amino acid mutations D239K, E240K, and K292D (numberings according to Kabat EU index). In one aspect, the heterodimerization approach described in WO 2007/110205 can be used alternatively.

In one aspect, the first subunit of the Fc domain comprises amino acid substitutions K392D and K409D, and the second subunit of the Fc domain comprises amino acid substitutions D356K and D399K (numbering according to Kabat EU index).

Fc Domain Modifications Reducing Fc Receptor Binding and/or Effector Function

The Fc domain confers to the antigen binding molecule(s) favorable pharmacokinetic properties, including a long serum half-life, which contributes to good accumulation in the target tissue and a favorable tissue-blood distribution ratio. At the same time it may, however, lead to undesirable targeting of the binding molecule(s) to cells expressing Fc receptors rather than to the preferred antigen-bearing cells. Moreover, the co-activation of Fc receptor signaling pathways may lead to cytokine release which may result in excessive activation of cytokine receptors and severe side effects upon systemic administration. Activation of (Fc receptor-bearing) immune cells other than T cells may even reduce efficacy of the pair of antigen binding molecules due to the potential destruction of T cells e.g. by NK cells.

Accordingly, in particular aspects, the Fc domain of the antigen binding molecule(s) according to the invention exhibits reduced binding affinity to an Fc receptor and/or reduced effector function, as compared to a native IgG₁ Fc domain. In some such aspects, the Fc domain (or the antigen binding molecule comprising said Fc domain) exhibits less than 50%, particularly less than 20%, more particularly less than 10% and most particularly less than 5% of the binding affinity to an Fc receptor, as compared to a native IgG₁ Fc domain (or a antigen binding molecule comprising a native IgG₁ Fc domain), and/or less than 50%, particularly less than 20%, more particularly less than 10% and most particularly less than 5% of the effector function, as compared to a native IgG₁ Fc domain domain (or a antigen binding molecule comprising a native IgG₁ Fc domain). In one aspect, the Fc domain domain (or the antigen binding molecule comprising said Fc domain) does not substantially bind to an Fc receptor and/or induce effector function. In particular aspects, the Fc receptor is an Fcγ receptor. In one aspect, the Fc receptor is a human Fc receptor. In one aspect, the Fc receptor is an activating Fc receptor. In specific aspects, the Fc receptor is an activating human Fcγ receptor, more specifically human FcγRIIIa, FcγRI or FcγRIIa, most specifically human FcγRIIIa. In one aspect, the effector function is one or more selected from the group of CDC, ADCC, ADCP, and cytokine secretion. In particular aspects, the effector function is ADCC. In one aspect, the Fc domain exhibits substantially similar binding affinity to neonatal Fc receptor (FcRn), as compared to a native IgG₁ Fc domain domain. Substantially similar binding to FcRn is achieved when the Fc domain (or the antigen binding molecule comprising said Fc domain) exhibits greater than about 70%, particularly greater than about 80%, more particularly greater than about 90% of the binding affinity of a native IgG₁ Fc domain (or the antigen binding molecule comprising a native IgG₁ Fc domain) to FcRn.

In certain aspects, the Fc domain is engineered to have reduced binding affinity to an Fc receptor and/or reduced effector function, as compared to a non-engineered Fc domain. In particular aspects, the Fc domain of the antigen binding molecule(s) comprises one or more amino acid mutation that reduces the binding affinity of the Fc domain to an Fc receptor and/or effector function. Typically, the same one or more amino acid mutation is present in each of the two subunits of the Fc domain. In one aspect, the amino acid mutation reduces the binding affinity of the Fc domain to an Fc receptor. In one aspect, the amino acid mutation reduces the binding affinity of the Fc domain to an Fc receptor by at least 2-fold, at least 5-fold, or at least 10-fold. In aspects where there is more than one amino acid mutation that reduces the binding affinity of the Fc domain to the Fc receptor, the combination of these amino acid mutations may reduce the binding affinity of the Fc domain to an Fc receptor by at least 10-fold, at least 20-fold, or even at least 50-fold. In one aspect, the antigen binding molecule(s) comprising an engineered Fc domain exhibits less than 20%, particularly less than 10%, more particularly less than 5% of the binding affinity to an Fc receptor as compared to a antigen binding molecule comprising a non-engineered Fc domain. In particular aspects, the Fc receptor is an Fcγ receptor. In one aspect, the Fc receptor is a human Fc receptor. In one aspect, the Fc receptor is an activating Fc receptor. In specific aspects, the Fc receptor is an activating human Fcγ receptor, more specifically human FcγRIIIa, FcγRI or FcγRIIa, most specifically human FcγRIIIa. Preferably, binding to each of these receptors is reduced. In one aspect, binding affinity to a complement component, specifically binding affinity to C1q, is also reduced. In one aspect, binding affinity to neonatal Fc receptor (FcRn) is not reduced.

Substantially similar binding to FcRn, i.e. preservation of the binding affinity of the Fc domain to said receptor, is achieved when the Fc domain (or the antigen binding molecule comprising said Fc domain) exhibits greater than about 70% of the binding affinity of a non-engineered form of the Fc domain (or the antigen binding molecule comprising said non-engineered form of the Fc domain) to FcRn. The Fc domain, or antigen binding molecule(s) of the invention comprising said Fc domain, may exhibit greater than about 80% and even greater than about 90% of such affinity. In certain aspects, the Fc domain of the antigen binding molecule(s) is engineered to have reduced effector function, as compared to a non-engineered Fc domain. The reduced effector function can include, but is not limited to, one or more of the following: reduced complement dependent cytotoxicity (CDC), reduced antibody dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced crosslinking of target-bound antibodies, reduced dendritic cell maturation, or reduced T cell priming. In one aspect, the reduced effector function is one or more selected from the group of reduced CDC, reduced ADCC, reduced ADCP, and reduced cytokine secretion. In particular aspects, the reduced effector function is reduced ADCC. In one aspect, the reduced ADCC is less than 20% of the ADCC induced by a non-engineered Fc domain (or a binding molecule comprising a non-engineered Fc domain).

In one aspect, the amino acid mutation that reduces the binding affinity of the Fc domain to an Fc receptor and/or effector function is an amino acid substitution. In one aspect, the Fc domain comprises an amino acid substitution at a position selected from the group of E233, L234, L235, N297, P331 and P329 (numberings according to Kabat EU index). In a more specific aspect, the Fc domain comprises an amino acid substitution at a position selected from the group of L234, L235 and P329 (numberings according to Kabat EU index). In one aspect, the Fc domain comprises the amino acid substitutions L234A and L235A (numberings according to Kabat EU index). In some such aspects, the Fc domain is an IgG₁ Fc domain, particularly a human IgG₁ Fc domain. In one aspect, the Fc domain comprises an amino acid substitution at position P329. In a more specific aspect, the amino acid substitution is P329A or P329G, particularly P329G (numberings according to Kabat EU index). In one aspect, the Fc domain comprises an amino acid substitution at position P329 and a further amino acid substitution at a position selected from E233, L234, L235, N297 and P331 (numberings according to Kabat EU index). In a more specific aspect, the further amino acid substitution is E233P, L234A, L235A, L235E, N297A, N297D or P331S. In particular aspects, the Fc domain comprises amino acid substitutions at positions P329, L234 and L235 (numberings according to Kabat EU index). In more particular aspects, the Fc domain comprises the amino acid mutations L234A, L235A and P329G (“P329G LALA”, “PGLALA” or “LALAPG”). Specifically, in particular aspects, each subunit of the Fc domain comprises the amino acid substitutions L234A, L235A and P329G (Kabat EU index numbering), i.e. in each of the first and the second subunit of the Fc domain the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index).

In some such aspects, the Fc domain is an IgG₁ Fc domain, particularly a human IgG₁ Fc domain. The “P329G LALA” combination of amino acid substitutions almost completely abolishes Fcγ receptor (as well as complement) binding of a human IgG₁ Fc domain, as described in PCT publication no. WO 2012/130831, which is incorporated herein by reference in its entirety. WO 2012/130831 also describes methods of preparing such mutant Fc domains and methods for determining its properties such as Fc receptor binding or effector functions.

IgG₄ antibodies exhibit reduced binding affinity to Fc receptors and reduced effector functions as compared to IgG₁ antibodies. Hence, in one aspect, the Fc domain of the binding molecule(s) of the invention is an IgG₄ Fc domain, particularly a human IgG₄ Fc domain. In one aspect, the IgG₄ Fc domain comprises an amino acid substitution at position S228, specifically the amino acid substitution S228P (numberings according to Kabat EU index). To further reduce its binding affinity to an Fc receptor and/or its effector function, in one aspect, the IgG₄ Fc domain comprises an amino acid substitution at position L235, specifically the amino acid substitution L235E (numberings according to Kabat EU index). In one aspect, the IgG₄ Fc domain comprises an amino acid substitution at position P329, specifically the amino acid substitution P329G (numberings according to Kabat EU index). In a preferred aspect, the IgG₄ Fc domain comprises amino acid substitutions at positions S228, L235 and P329, specifically amino acid substitutions S228P, L235E and P329G (numberings according to Kabat EU index). Such IgG₄ Fc domain mutants and their Fcγ receptor binding properties are described in PCT publication no. WO 2012/130831, incorporated herein by reference in its entirety.

In particular aspects, the Fc domain exhibiting reduced binding affinity to an Fc receptor and/or reduced effector function, as compared to a native IgG₁ Fc domain, is a human IgG₁ Fc domain comprising the amino acid substitutions L234A, L235A and optionally P329G, or a human IgG₄ Fc domain comprising the amino acid substitutions S228P, L235E and optionally P329G (numberings according to Kabat EU index).

In certain aspects, N-glycosylation of the Fc domain has been eliminated. In some such aspects, the Fc domain comprises an amino acid mutation at position N297, particularly an amino acid substitution replacing asparagine by alanine (N297A) or aspartic acid (N297D) (numberings according to Kabat EU index).

In addition to the Fc domains described hereinabove and in PCT publication no. WO 2012/130831, Fc domains with reduced Fc receptor binding and/or effector function also include those with substitution of one or more of Fc domain residues 238, 265, 269, 270, 297, 327 and 329 (U.S. Pat. No. 6,737,056) (numberings according to Kabat EU index). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Mutant Fc domains can be prepared by amino acid deletion, substitution, insertion or modification using genetic or chemical methods well known in the art. Genetic methods may include site specific mutagenesis of the encoding DNA sequence, PCR, gene synthesis, and the like. The correct nucleotide changes can be verified for example by sequencing.

Binding to Fc receptors can be easily determined e.g. by ELISA, or by Surface Plasmon Resonance (SPR) using standard instrumentation such as a BIAcore instrument (GE Healthcare), and Fc receptors such as may be obtained by recombinant expression. Alternatively, binding affinity of Fc domains or binding molecule(s) comprising an Fc domain for Fc receptors may be evaluated using cell lines known to express particular Fc receptors, such as human NK cells expressing FcγIIIa receptor.

Effector function of an Fc domain, or a binding molecule comprising an Fc domain, can be measured by methods known in the art. Examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987).

Alternatively, non-radioactive assays may be employed (see, for example, ACTI™ nonradioactive cytotoxicity assay for flow cytometry (CellTechnology, Inc. Mountain View, CA); and CytoTox 96® non-radioactive cytotoxicity assay (Promega, Madison, WI)). Useful effector cells for such assays include peripheral blood mononuclear cells (PBMC) and Natural Killer (NK) cells.

Alternatively, or additionally, ADCC activity of the molecule of interest may be assessed in vivo, e.g. in an animal model such as that disclosed in Clynes et al., Proc Natl Acad Sci USA 95, 652-656 (1998).

In one aspect, binding of the Fc domain to a complement component, specifically to C1q, is reduced. Accordingly, in one aspect wherein the Fc domain is engineered to have reduced effector function, said reduced effector function includes reduced CDC. C1q binding assays may be carried out to determine whether the Fc domain, or the binding molecule comprising the Fc domain, is able to bind C1q and hence has CDC activity. See e.g., C1q and C3c binding ELISA in WO 2006/029879 and WO 2005/100402. To assess complement activation, a CDC assay may be performed (see, for example, Gazzano-Santoro et al., J Immunol Methods 202, 163 (1996); Cragg et al., Blood 101, 1045-1052 (2003); and Cragg and Glennie, Blood 103, 2738-2743 (2004)).

FcRn binding and in vivo clearance/half life determinations can also be performed using methods known in the art (see, e.g., Petkova, S. B. et al., Int'l. Immunol. 18(12):1759-1769 (2006); WO 2013/120929).

### Configuration of the Antigen Binding Molecules

The antigen binding molecule(s) according to the present invention may have various molecular configurations, i.e. the domains of the binding molecule(s) may be linked to each other in different ways.

The antigen binding molecule(s) may comprise a target-binding domain, a cytokine receptor-binding domain and a Fc domain, wherein the cytokine receptor-binding domain is fused at its C-terminus to the N-terminus of the target-binding domain, and the target-binding domain is fused at its C-terminus to the N-terminus of one of the Fc domain subunits. The target-binding domain may be a Fab molecule. Thus, the cytokine receptor-binding domain may be fused to the VH or VL domain of the target binding-domain. In one embodiment, the antigen binding molecule(s) comprise a target-binding domain, a cytokine receptor-binding domain and a Fc domain, wherein the target-binding domain is a Fab molecule, and wherein the cytokine receptor-binding domain is fused at its C-terminus to the N-terminus of the VH domain of the target-binding domain, and the target-binding domain is fused at its C-terminus to the N-terminus of one of the Fc domain subunits (see e.g. FIGS. 11B, 11C, 11F and 11G for schematic illustration). In one embodiment, the antigen binding molecule(s) comprise a target-binding domain, a cytokine receptor-binding domain and a Fc domain, wherein the target-binding domain is a Fab molecule, and wherein the cytokine receptor-binding domain is fused at its C-terminus to the N-terminus of the VL domain of the target-binding domain, and the target-binding domain is fused at its C-terminus to the N-terminus of one of the Fc domain subunits (see e.g. FIG. 11D, 11E, 11H and 11I for schematic illustration).

The antigen binding molecule(s) may comprise a target-binding domain, a cytokine receptor-binding domain and a Fc domain, wherein the target-binding domain is fused at its C-terminus to the N-terminus of the first Fc domain subunit and the cytokine receptor-binding domain is fused at its C-terminus to the N-terminus of the second Fc domain subunit (see e.g. FIG. 13A, 13B, 13C and 13D for schematic illustration).

The antigen binding molecule(s) may comprise a target-binding domain, a cytokine receptor-binding domain and a Fc domain, wherein the target-binding domain is fused at its C-terminus to the N-terminus of one of the Fc domain subunits and the cytokine receptor-binding domain is fused at its N-terminus to the C-terminus of the same Fc domain subunit (see e.g. FIG. 13E, 13F, 13G and 13H for schematic illustration).

The cytokine-receptor binding domain may be fused via a peptide linker. The peptide linker may comprise an amino acid sequence according to SEQ ID NO: 262, SEQ ID NO: 263, SEQ ID NO: 264, SEQ ID NO: 279, SEQ ID NO: 280 or SEQ ID NO: 281.

### Pairs of Antigen Binding Molecules

In one aspect, both antigen binding molecules of the pair of antigen binding molecules comprise a target-binding domain, a cytokine receptor-binding domain and a Fc domain, wherein the cytokine receptor-binding domain is fused at its C-terminus to the N-terminus of the target-binding domain, and the target-binding domain is fused at its C-terminus to the N-terminus of one of the Fc domain subunits.

In one aspect, the target-binding domains of the antigen binding molecules are Fab molecules. Thus, in one aspect the pair of antigen binding molecules comprises a first antigen binding molecule comprising a first polypeptide comprising a first Fc domain subunit, a second polypeptide comprising in order from the N-terminus to C-terminus a first cytokine receptor-binding domain, VH₁, CH1₁ and a second Fc domain subunit, and a third polypeptide comprising in order from the N-terminus to C-terminus VL₁ and CL₁; and a second antigen binding molecule comprising a first polypeptide comprising a first Fc domain subunit, a second polypeptide comprising in order from the N-terminus to C-terminus a second cytokine receptor-binding domain, VH₂, CH1₂ and a second Fc domain subunit, and a third polypeptide comprising in order from the N-terminus to C-terminus VL₂ and CL₂.

In one aspect, the pair of antigen binding molecules comprises first antigen binding molecule comprising a first polypeptide comprising a first Fc domain subunit, a second polypeptide comprising in order from the N-terminus to C-terminus VH₁, CH1₁ and a second Fc domain subunit, and a third polypeptide comprising in order from the N-terminus to C-terminus a first cytokine receptor-binding domain, VL₁ and CL₁; and a second antigen binding molecule comprising a first polypeptide comprising a first Fc domain subunit, a second polypeptide comprising in order from the N-terminus to C-terminus VH₂, CH1₂ and a second Fc domain subunit; a third polypeptide comprising in order from the N-terminus to C-terminus a second cytokine receptor-binding domain, VL₂ and CL₂.

In one aspect, the pair of antigen binding molecules comprises first antigen binding molecule comprising a first polypeptide comprising a first Fc domain subunit, a second polypeptide comprising in order from the N-terminus to C-terminus a first cytokine receptor-binding domain, VH₁, CH1₁ and a second Fc domain subunit, and a third polypeptide comprising in order from the N-terminus to C-terminus VL₁ and CL₁; and a second antigen binding molecule comprising a first polypeptide comprising a first Fc domain subunit, a second polypeptide comprising in order from the N-terminus to C-terminus VH₂, CH1₂ and a second Fc domain subunit; a third polypeptide comprising in order from the N-terminus to C-terminus a second cytokine receptor-binding domain, VL₂ and CL₂.

In one aspect, the pair of antigen binding molecules comprises first antigen binding molecule comprising a first polypeptide comprising a first Fc domain subunit, a second polypeptide comprising in order from the N-terminus to C-terminus a first cytokine receptor-binding domain, VL₁, CH1₁ and a second Fc domain subunit, and a third polypeptide comprising in order from the N-terminus to C-terminus VH₁ and CL₁; and a second antigen binding molecule comprises a first polypeptide comprising a first Fc domain subunit, a second polypeptide comprising in order from the N-terminus to C-terminus a second cytokine receptor-binding domain, VH₂, CH1₂ and a second Fc domain subunit, and a third polypeptide comprising in order from the N-terminus to C-terminus VL₂ and CL₂.

In one aspect, the pair of antigen binding molecules comprises first antigen binding molecule comprising a first polypeptide comprising a first Fc domain subunit, a second polypeptide comprising in order from the N-terminus to C-terminus VL₁, CH1₁ and a second Fc domain subunit, and a third polypeptide comprising in order from the N-terminus to C-terminus a first cytokine receptor-binding domain, VH₁ and CL₁; and a second antigen binding molecule comprises a first polypeptide comprising a first Fc domain subunit, a second polypeptide comprising in order from the N-terminus to C-terminus VH₂, CH1₂ and a second Fc domain subunit; a third polypeptide comprising in order from the N-terminus to C-terminus a second cytokine receptor-binding domain, VL₂ and CL₂.

In one aspect, the pair of antigen binding molecules comprises first antigen binding molecule comprising a first polypeptide comprising a first Fc domain subunit, a second polypeptide comprising in order from the N-terminus to C-terminus a first cytokine receptor-binding domain, VL₁, CH1₁ and a second Fc domain subunit, and a third polypeptide comprising in order from the N-terminus to C-terminus VH₁ and CL₁; and a second antigen binding molecule comprises a first polypeptide comprising a first Fc domain subunit, a second polypeptide comprising in order from the N-terminus to C-terminus VH₂, CH1₂ and a second Fc domain subunit, and a third polypeptide comprising in order from the N-terminus to C-terminus a second cytokine receptor-binding domain, VL₂ and CL₂.

In one aspect, the pair of antigen binding molecules comprises first antigen binding molecule comprising a first polypeptide comprising a first Fc domain subunit, a second polypeptide comprising in order from the N-terminus to C-terminus a VL₁, CH1₁ and a second Fc domain subunit, and a third polypeptide comprising in order from the N-terminus to C-terminus a first cytokine receptor-binding domain, VH₁ and CL₁; and a second antigen binding molecule comprises a first polypeptide comprising a first Fc domain subunit, a second polypeptide comprising in order from the N-terminus to C-terminus a second cytokine receptor-binding domain, VH₂, CH1₂ and a second Fc domain subunit, and a third polypeptide comprising in order from the N-terminus to C-terminus VL₂ and CL₂.

In one aspect, the pair of antigen binding molecules comprises first antigen binding molecule comprising a first polypeptide comprising an amino acid sequence of SEQ ID NO: 28, a second polypeptide comprising an amino acid sequence of SEQ ID NO: 27 and a third polypeptide comprising an amino acid sequence of SEQ ID NO: 25, and a second antigen binding molecule comprising a first polypeptide comprising an amino acid sequence of SEQ ID NO: 28, a second polypeptide comprising an amino acid sequence of SEQ ID NO: 30 and a third polypeptide comprising an amino acid sequence of SEQ ID NO: 29.

In one aspect, the pair of antigen binding molecules comprises first antigen binding molecule comprising a first polypeptide comprising an amino acid sequence of SEQ ID NO: 28, a second polypeptide comprising an amino acid sequence of SEQ ID NO: 38 and a third polypeptide comprising an amino acid sequence of SEQ ID NO: 25, and a second antigen binding molecule comprising a first polypeptide comprising an amino acid sequence of SEQ ID NO: 28, a second polypeptide comprising an amino acid sequence of SEQ ID NO: 37 and a third polypeptide comprising an amino acid sequence of SEQ ID NO: 29.

In one aspect, the pair of antigen binding molecules comprises first antigen binding molecule comprising a first polypeptide comprising an amino acid sequence of SEQ ID NO: 28, a second polypeptide comprising an amino acid sequence of SEQ ID NO: 32 and a third polypeptide comprising an amino acid sequence of SEQ ID NO: 31, and a second antigen binding molecule comprising a first polypeptide comprising an amino acid sequence of SEQ ID NO: 28, a second polypeptide comprising an amino acid sequence of SEQ ID NO: 34 and a third polypeptide comprising an amino acid sequence of SEQ ID NO: 33.

In one aspect, the pair of antigen binding molecules comprises first antigen binding molecule comprising a first polypeptide comprising an amino acid sequence of SEQ ID NO: 28, a second polypeptide comprising an amino acid sequence of SEQ ID NO: 32 and a third polypeptide comprising an amino acid sequence of SEQ ID NO: 35, and a second antigen binding molecule comprising a first polypeptide comprising an amino acid sequence of SEQ ID NO: 28, a second polypeptide comprising an amino acid sequence of SEQ ID NO: 34 and a third polypeptide comprising an amino acid sequence of SEQ ID NO: 36.

In one aspect, the pair of antigen binding molecules comprises first antigen binding molecule comprising a first polypeptide comprising an amino acid sequence of SEQ ID NO: 28, a second polypeptide comprising an amino acid sequence of SEQ ID NO: 27 and a third polypeptide comprising an amino acid sequence of SEQ ID NO: 25, and a second antigen binding molecule comprising a first polypeptide comprising an amino acid sequence of SEQ ID NO: 28, a second polypeptide comprising an amino acid sequence of SEQ ID NO: 34 and a third polypeptide comprising an amino acid sequence of SEQ ID NO: 33.

In one aspect, the pair of antigen binding molecules comprises first antigen binding molecule comprising a first polypeptide comprising an amino acid sequence of SEQ ID NO: 28, a second polypeptide comprising an amino acid sequence of SEQ ID NO: 30 and a third polypeptide comprising an amino acid sequence of SEQ ID NO: 29, and a second antigen binding molecule comprising a first polypeptide comprising an amino acid sequence of SEQ ID NO: 28, a second polypeptide comprising an amino acid sequence of SEQ ID NO: 32 and a third polypeptide comprising an amino acid sequence of SEQ ID NO: 31.

In one aspect, the pair of antigen binding molecules comprises first antigen binding molecule comprising a first polypeptide comprising an amino acid sequence of SEQ ID NO: 28, a second polypeptide comprising an amino acid sequence of SEQ ID NO: 38 and a third polypeptide comprising an amino acid sequence of SEQ ID NO: 25, and a second antigen binding molecule comprising a first polypeptide comprising an amino acid sequence of SEQ ID NO: 28, a second polypeptide comprising an amino acid sequence of SEQ ID NO: 34 and a third polypeptide comprising an amino acid sequence of SEQ ID NO: 36.

In one aspect, the pair of antigen binding molecules comprises first antigen binding molecule comprising a first polypeptide comprising an amino acid sequence of SEQ ID NO: 28, a second polypeptide comprising an amino acid sequence of SEQ ID NO: 37 and a third polypeptide comprising an amino acid sequence of SEQ ID NO: 29, and a second antigen binding molecule comprising a first polypeptide comprising an amino acid sequence of SEQ ID NO: 28, a second polypeptide comprising an amino acid sequence of SEQ ID NO: 32 and a third polypeptide comprising an amino acid sequence of SEQ ID NO: 35.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 260 and a third polypeptide comprising an amino acid sequence of SEQ ID NO: 124, and a second antigen binding molecule comprising a first polypeptide comprising an amino acid sequence of SEQ ID NO: 106, a second polypeptide comprising an amino acid sequence of SEQ ID NO: 259 and a third polypeptide comprising an amino acid sequence of SEQ ID NO: 127.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 119 and a third polypeptide comprising an amino acid sequence of SEQ ID NO: 256, and a second antigen binding molecule comprising a first polypeptide comprising an amino acid sequence of SEQ ID NO: 106, a second polypeptide comprising an amino acid sequence of SEQ ID NO: 122 and a third polypeptide comprising an amino acid sequence of SEQ ID NO: 255.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 261 and a third polypeptide comprising an amino acid sequence of SEQ ID NO: 124, and a second antigen binding molecule comprising a first polypeptide comprising an amino acid sequence of SEQ ID NO: 106, a second polypeptide comprising an amino acid sequence of SEQ ID NO: 258 and a third polypeptide comprising an amino acid sequence of SEQ ID NO: 127.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 119 and a third polypeptide comprising an amino acid sequence of SEQ ID NO: 257, and a second antigen binding molecule comprising a first polypeptide comprising an amino acid sequence of SEQ ID NO: 106, a second polypeptide comprising an amino acid sequence of SEQ ID NO: 112 and a third polypeptide comprising an amino acid sequence of SEQ ID NO: 254.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 260 and a third polypeptide comprising an amino acid sequence of SEQ ID NO: 124, and a second antigen binding molecule comprising a first polypeptide comprising an amino acid sequence of SEQ ID NO: 106, a second polypeptide comprising an amino acid sequence of SEQ ID NO: 112 and a third polypeptide comprising an amino acid sequence of SEQ ID NO: 255.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 119 and a third polypeptide comprising an amino acid sequence of SEQ ID NO: 256, and a second antigen binding molecule comprising a first polypeptide comprising an amino acid sequence of SEQ ID NO: 106, a second polypeptide comprising an amino acid sequence of SEQ ID NO: 259 and a third polypeptide comprising an amino acid sequence of SEQ ID NO: 127.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 261 and a third polypeptide comprising an amino acid sequence of SEQ ID NO: 124, and a second antigen binding molecule comprising a first polypeptide comprising an amino acid sequence of SEQ ID NO: 106, a second polypeptide comprising an amino acid sequence of SEQ ID NO: 112 and a third polypeptide comprising an amino acid sequence of SEQ ID NO: 254.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 119 and a third polypeptide comprising an amino acid sequence of SEQ ID NO: 257, and a second antigen binding molecule comprising a first polypeptide comprising an amino acid sequence of SEQ ID NO: 106, a second polypeptide comprising an amino acid sequence of SEQ ID NO: 258 and a third polypeptide comprising an amino acid sequence of SEQ ID NO: 127.

In one aspect, the pair of antigen binding molecules comprises first antigen binding molecule comprising a first polypeptide comprising an amino acid sequence of SEQ ID NO: 86, a second polypeptide comprising an amino acid sequence of SEQ ID NO: 44 and a third polypeptide comprising an amino acid sequence of SEQ ID NO: 83, and a second antigen binding molecule comprising a first polypeptide comprising an amino acid sequence of SEQ ID NO: 28, a second polypeptide comprising an amino acid sequence of SEQ ID NO: 88 and a third polypeptide comprising an amino acid sequence of SEQ ID NO: 87.

In one aspect, the pair of antigen binding molecules comprises first antigen binding molecule comprising a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89, and a second antigen binding molecule comprising a first polypeptide comprising an amino acid sequence of SEQ ID NO: 92, a second polypeptide comprising an amino acid sequence of SEQ ID NO: 44 and a third polypeptide comprising an amino acid sequence of SEQ ID NO: 91.

In one aspect, the pair of antigen binding molecules comprises first antigen binding molecule comprising a first polypeptide comprising an amino acid sequence of SEQ ID NO: 28, a second polypeptide comprising an amino acid sequence of SEQ ID NO: 96 and a third polypeptide comprising an amino acid sequence of SEQ ID NO: 95, and a second antigen binding molecule comprising a first polypeptide comprising an amino acid sequence of SEQ ID NO: 28, a second polypeptide comprising an amino acid sequence of SEQ ID NO: 101 and a third polypeptide comprising an amino acid sequence of SEQ ID NO: 89.

In one aspect, the pair of antigen binding molecules comprises first antigen binding molecule comprising a first polypeptide comprising an amino acid sequence of SEQ ID NO: 28, a second polypeptide comprising an amino acid sequence of SEQ ID NO: 99 and a third polypeptide comprising an amino acid sequence of SEQ ID NO: 98, and a second antigen binding molecule comprising a first polypeptide comprising an amino acid sequence of SEQ ID NO: 28, a second polypeptide comprising an amino acid sequence of SEQ ID NO: 88 and a third polypeptide comprising an amino acid sequence of SEQ ID NO: 102.

In one aspect, the pair of antigen binding molecules comprises first antigen binding molecule comprising a first polypeptide comprising an amino acid sequence of SEQ ID NO: 28, a second polypeptide comprising an amino acid sequence of SEQ ID NO: 96 and a third polypeptide comprising an amino acid sequence of SEQ ID NO: 97, and a second antigen binding molecule comprising a first polypeptide comprising an amino acid sequence of SEQ ID NO: 28, a second polypeptide comprising an amino acid sequence of SEQ ID NO: 103 and a third polypeptide comprising an amino acid sequence of SEQ ID NO: 89.

In one aspect, the pair of antigen binding molecules comprises first antigen binding molecule comprising a first polypeptide comprising an amino acid sequence of SEQ ID NO: 28, a second polypeptide comprising an amino acid sequence of SEQ ID NO: 100 and a third polypeptide comprising an amino acid sequence of SEQ ID NO: 98, and a second antigen binding molecule comprising a first polypeptide comprising an amino acid sequence of SEQ ID NO: 28, a second polypeptide comprising an amino acid sequence of SEQ ID NO: 88 and a third polypeptide comprising an amino acid sequence of SEQ ID NO: 104.

In one aspect, the pair of antigen binding molecules comprises first antigen binding molecule comprising a first polypeptide comprising an amino acid sequence of SEQ ID NO: 28, a second polypeptide comprising an amino acid sequence of SEQ ID NO: 98 and a third polypeptide comprising an amino acid sequence of SEQ ID NO: 99, and a second antigen binding molecule comprising a first polypeptide comprising an amino acid sequence of SEQ ID NO: 28, a second polypeptide comprising an amino acid sequence of SEQ ID NO: 101 and a third polypeptide comprising an amino acid sequence of SEQ ID NO: 89.

In one aspect, the pair of antigen binding molecules comprises first antigen binding molecule comprising a first polypeptide comprising an amino acid sequence of SEQ ID NO: 28, a second polypeptide comprising an amino acid sequence of SEQ ID NO: 96 and a third polypeptide comprising an amino acid sequence of SEQ ID NO: 95, and a second antigen binding molecule comprising a first polypeptide comprising an amino acid sequence of SEQ ID NO: 28, a second polypeptide comprising an amino acid sequence of SEQ ID NO: 88 and a third polypeptide comprising an amino acid sequence of SEQ ID NO: 102.

In one aspect, the pair of antigen binding molecules comprises first antigen binding molecule comprising a first polypeptide comprising an amino acid sequence of SEQ ID NO: 28, a second polypeptide comprising an amino acid sequence of SEQ ID NO: 100 and a third polypeptide comprising an amino acid sequence of SEQ ID NO: 98, and a second antigen binding molecule comprising a first polypeptide comprising an amino acid sequence of SEQ ID NO: 28, a second polypeptide comprising an amino acid sequence of SEQ ID NO: 103 and a third polypeptide comprising an amino acid sequence of SEQ ID NO: 89.

In one aspect, the pair of antigen binding molecules comprises first antigen binding molecule comprising a first polypeptide comprising an amino acid sequence of SEQ ID NO: 28, a second polypeptide comprising an amino acid sequence of SEQ ID NO: 96 and a third polypeptide comprising an amino acid sequence of SEQ ID NO: 97, and a second antigen binding molecule comprising a first polypeptide comprising an amino acid sequence of SEQ ID NO: 28, a second polypeptide comprising an amino acid sequence of SEQ ID NO: 88 and a third polypeptide comprising an amino acid sequence of SEQ ID NO: 104.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 110 and a third polypeptide comprising an amino acid sequence of SEQ ID NO: 25, and a second antigen binding molecule comprising a first polypeptide comprising an amino acid sequence of SEQ ID NO: 106, a second polypeptide comprising an amino acid sequence of SEQ ID NO: 113 and a third polypeptide comprising an amino acid sequence of SEQ ID NO: 29.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 32 and a third polypeptide comprising an amino acid sequence of SEQ ID NO: 105, and a second antigen binding molecule comprising a first polypeptide comprising an amino acid sequence of SEQ ID NO: 106, a second polypeptide comprising an amino acid sequence of SEQ ID NO: 34 and a third polypeptide comprising an amino acid sequence of SEQ ID NO: 109.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 111 and a third polypeptide comprising an amino acid sequence of SEQ ID NO: 25, and a second antigen binding molecule comprising a first polypeptide comprising an amino acid sequence of SEQ ID NO: 106, a second polypeptide comprising an amino acid sequence of SEQ ID NO: 112 and a third polypeptide comprising an amino acid sequence of SEQ ID NO: 29.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 32 and a third polypeptide comprising an amino acid sequence of SEQ ID NO: 107, and a second antigen binding molecule comprising a first polypeptide comprising an amino acid sequence of SEQ ID NO: 106, a second polypeptide comprising an amino acid sequence of SEQ ID NO: 34 and a third polypeptide comprising an amino acid sequence of SEQ ID NO: 108.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 110 and a third polypeptide comprising an amino acid sequence of SEQ ID NO: 25, and a second antigen binding molecule comprising a first polypeptide comprising an amino acid sequence of SEQ ID NO: 106, a second polypeptide comprising an amino acid sequence of SEQ ID NO: 34 and a third polypeptide comprising an amino acid sequence of SEQ ID NO: 109.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 32 and a third polypeptide comprising an amino acid sequence of SEQ ID NO: 105, and a second antigen binding molecule comprising a first polypeptide comprising an amino acid sequence of SEQ ID NO: 106, a second polypeptide comprising an amino acid sequence of SEQ ID NO: 113 and a third polypeptide comprising an amino acid sequence of SEQ ID NO: 29.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 111 and a third polypeptide comprising an amino acid sequence of SEQ ID NO: 25, and a second antigen binding molecule comprising a first polypeptide comprising an amino acid sequence of SEQ ID NO: 106, a second polypeptide comprising an amino acid sequence of SEQ ID NO: 34 and a third polypeptide comprising an amino acid sequence of SEQ ID NO: 108.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 32 and a third polypeptide comprising an amino acid sequence of SEQ ID NO: 107, and a second antigen binding molecule comprising a first polypeptide comprising an amino acid sequence of SEQ ID NO: 106, a second polypeptide comprising an amino acid sequence of SEQ ID NO: 112 and a third polypeptide comprising an amino acid sequence of SEQ ID NO: 29.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 125 and a third polypeptide comprising an amino acid sequence of SEQ ID NO: 124, and a second antigen binding molecule comprising a first polypeptide comprising an amino acid sequence of SEQ ID NO: 106, a second polypeptide comprising an amino acid sequence of SEQ ID NO: 129 and a third polypeptide comprising an amino acid sequence of SEQ ID NO: 127.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 119 and a third polypeptide comprising an amino acid sequence of SEQ ID NO: 118, and a second antigen binding molecule comprising a first polypeptide comprising an amino acid sequence of SEQ ID NO: 106, a second polypeptide comprising an amino acid sequence of SEQ ID NO: 122 and a third polypeptide comprising an amino acid sequence of SEQ ID NO: 123.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 126 and a third polypeptide comprising an amino acid sequence of SEQ ID NO: 124, and a second antigen binding molecule comprising a first polypeptide comprising an amino acid sequence of SEQ ID NO: 106, a second polypeptide comprising an amino acid sequence of SEQ ID NO: 128 and a third polypeptide comprising an amino acid sequence of SEQ ID NO: 127.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 119 and a third polypeptide comprising an amino acid sequence of SEQ ID NO: 120, and a second antigen binding molecule comprising a first polypeptide comprising an amino acid sequence of SEQ ID NO: 106, a second polypeptide comprising an amino acid sequence of SEQ ID NO: 122 and a third polypeptide comprising an amino acid sequence of SEQ ID NO: 121.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 125 and a third polypeptide comprising an amino acid sequence of SEQ ID NO: 124, and a second antigen binding molecule comprising a first polypeptide comprising an amino acid sequence of SEQ ID NO: 106, a second polypeptide comprising an amino acid sequence of SEQ ID NO: 122 and a third polypeptide comprising an amino acid sequence of SEQ ID NO: 123.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 119 and a third polypeptide comprising an amino acid sequence of SEQ ID NO: 118, and a second antigen binding molecule comprising a first polypeptide comprising an amino acid sequence of SEQ ID NO: 106, a second polypeptide comprising an amino acid sequence of SEQ ID NO: 129 and a third polypeptide comprising an amino acid sequence of SEQ ID NO: 127.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 126 and a third polypeptide comprising an amino acid sequence of SEQ ID NO: 124, and a second antigen binding molecule comprising a first polypeptide comprising an amino acid sequence of SEQ ID NO: 106, a second polypeptide comprising an amino acid sequence of SEQ ID NO: 122 and a third polypeptide comprising an amino acid sequence of SEQ ID NO: 121.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 119 and a third polypeptide comprising an amino acid sequence of SEQ ID NO: 120, and a second antigen binding molecule comprising a first polypeptide comprising an amino acid sequence of SEQ ID NO: 106, a second polypeptide comprising an amino acid sequence of SEQ ID NO: 128 and a third polypeptide comprising an amino acid sequence of SEQ ID NO: 127.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 136 and a third polypeptide comprising an amino acid sequence of SEQ ID NO: 134, and a second antigen binding molecule comprising a first polypeptide comprising an amino acid sequence of SEQ ID NO: 106, a second polypeptide comprising an amino acid sequence of SEQ ID NO: 138 and a third polypeptide comprising an amino acid sequence of SEQ ID NO: 137.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 135 and a third polypeptide comprising an amino acid sequence of SEQ ID NO: 134, and a second antigen binding molecule comprising a first polypeptide comprising an amino acid sequence of SEQ ID NO: 106, a second polypeptide comprising an amino acid sequence of SEQ ID NO: 138 and a third polypeptide comprising an amino acid sequence of SEQ ID NO: 139.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 146 and a third polypeptide comprising an amino acid sequence of SEQ ID NO: 144, and a second antigen binding molecule comprising a first polypeptide comprising an amino acid sequence of SEQ ID NO: 106, a second polypeptide comprising an amino acid sequence of SEQ ID NO: 148 and a third polypeptide comprising an amino acid sequence of SEQ ID NO: 147.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 145 and a third polypeptide comprising an amino acid sequence of SEQ ID NO: 144, and a second antigen binding molecule comprising a first polypeptide comprising an amino acid sequence of SEQ ID NO: 106, a second polypeptide comprising an amino acid sequence of SEQ ID NO: 148 and a third polypeptide comprising an amino acid sequence of SEQ ID NO: 149.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 155 and a third polypeptide comprising an amino acid sequence of SEQ ID NO: 154, and a second antigen binding molecule comprising a first polypeptide comprising an amino acid sequence of SEQ ID NO: 106, a second polypeptide comprising an amino acid sequence of SEQ ID NO: 165 and a third polypeptide comprising an amino acid sequence of SEQ ID NO: 163.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 161 and a third polypeptide comprising an amino acid sequence of SEQ ID NO: 160, and a second antigen binding molecule comprising a first polypeptide comprising an amino acid sequence of SEQ ID NO: 106, a second polypeptide comprising an amino acid sequence of SEQ ID NO: 158 and a third polypeptide comprising an amino acid sequence of SEQ ID NO: 159.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 155 and a third polypeptide comprising an amino acid sequence of SEQ ID NO: 156, and a second antigen binding molecule comprising a first polypeptide comprising an amino acid sequence of SEQ ID NO: 106, a second polypeptide comprising an amino acid sequence of SEQ ID NO: 164 and a third polypeptide comprising an amino acid sequence of SEQ ID NO: 163.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 162 and a third polypeptide comprising an amino acid sequence of SEQ ID NO: 160, and a second antigen binding molecule comprising a first polypeptide comprising an amino acid sequence of SEQ ID NO: 106, a second polypeptide comprising an amino acid sequence of SEQ ID NO: 158 and a third polypeptide comprising an amino acid sequence of SEQ ID NO: 157.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 161 and a third polypeptide comprising an amino acid sequence of SEQ ID NO: 160, and a second antigen binding molecule comprising a first polypeptide comprising an amino acid sequence of SEQ ID NO: 106, a second polypeptide comprising an amino acid sequence of SEQ ID NO: 165 and a third polypeptide comprising an amino acid sequence of SEQ ID NO: 163.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 155 and a third polypeptide comprising an amino acid sequence of SEQ ID NO: 154, and a second antigen binding molecule comprising a first polypeptide comprising an amino acid sequence of SEQ ID NO: 106, a second polypeptide comprising an amino acid sequence of SEQ ID NO: 158 and a third polypeptide comprising an amino acid sequence of SEQ ID NO: 159.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 162 and a third polypeptide comprising an amino acid sequence of SEQ ID NO: 160, and a second antigen binding molecule comprising a first polypeptide comprising an amino acid sequence of SEQ ID NO: 106, a second polypeptide comprising an amino acid sequence of SEQ ID NO: 164 and a third polypeptide comprising an amino acid sequence of SEQ ID NO: 163.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 155 and a third polypeptide comprising an amino acid sequence of SEQ ID NO: 156, and a second antigen binding molecule comprising a first polypeptide comprising an amino acid sequence of SEQ ID NO: 106, a second polypeptide comprising an amino acid sequence of SEQ ID NO: 158 and a third polypeptide comprising an amino acid sequence of SEQ ID NO: 157.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 194 and a third polypeptide comprising an amino acid sequence of SEQ ID NO: 193, and a second antigen binding molecule comprising a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 195 and a third polypeptide comprising an amino acid sequence of SEQ ID NO: 124, and a second antigen binding molecule comprising a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 196 and a third polypeptide comprising an amino acid sequence of SEQ ID NO: 124, and a second antigen binding molecule comprising a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 197 and a third polypeptide comprising an amino acid sequence of SEQ ID NO: 124, and a second antigen binding molecule comprising a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 198 and a third polypeptide comprising an amino acid sequence of SEQ ID NO: 124, and a second antigen binding molecule comprising a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 199 and a third polypeptide comprising an amino acid sequence of SEQ ID NO: 124, and a second antigen binding molecule comprising a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 200 and a third polypeptide comprising an amino acid sequence of SEQ ID NO: 124, and a second antigen binding molecule comprising a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 201 and a third polypeptide comprising an amino acid sequence of SEQ ID NO: 124, and a second antigen binding molecule comprising a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 202 and a third polypeptide comprising an amino acid sequence of SEQ ID NO: 124, and a second antigen binding molecule comprising a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 203 and a third polypeptide comprising an amino acid sequence of SEQ ID NO: 124, and a second antigen binding molecule comprising a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 204 and a third polypeptide comprising an amino acid sequence of SEQ ID NO: 124, and a second antigen binding molecule comprising a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89.

In one aspect, the pair of antigen binding molecules comprises first antigen binding molecule comprising a first polypeptide comprising an amino acid sequence of SEQ ID NO: 106, a second polypeptide comprising an amino acid sequence of SEQ ID NO: 205 and a third polypeptide comprising an amino acid sequence of SEQ ID NO: 124, and a second antigen binding molecule comprising a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89.

In one aspect, both antigen binding molecules of the pair of antigen binding molecules comprise a target-binding domain, a cytokine receptor-binding domain and a Fc domain, wherein the target-binding domain is fused at its C-terminus to the N-terminus of the first Fc domain subunit and the cytokine receptor-binding domain is fused at its C-terminus to the N-terminus of the second Fc domain subunit. In one aspect, both antigen binding molecules of the pair of antigen binding molecules comprise a target-binding domain, a cytokine receptor-binding domain and a Fc domain, wherein the target-binding domain is fused at its C-terminus to the N-terminus of the first Fc domain subunit and the cytokine receptor-binding domain is fused at its C-terminus to the N-terminus of the second Fc domain subunit, wherein the target-binding domain is a Fab molecule. In one aspect, the pair of antigen binding molecules comprises first antigen binding molecule comprising a first polypeptide comprising in order from the N-terminus to C-terminus VH₁, CH1₁ and a first Fc domain subunit, a second polypeptide comprising in order from the N-terminus to C-terminus VL₁ and CL₁, and a third polypeptide comprising in order from the N-terminus to C-terminus a first cytokine receptor-binding domain and a second Fc domain subunit, and a second antigen binding molecule comprises a first polypeptide comprising in order from the N-terminus to C-terminus VH₂, CH1₂ and a first Fc domain subunit, a second polypeptide comprising in order from the N-terminus to C-terminus VL₂ and CL₂, and a third polypeptide comprising in order from the N-terminus to C-terminus a second cytokine receptor-binding domain and a second Fc domain subunit.

In one aspect, the pair of antigen binding molecules comprises first antigen binding molecule comprising a first polypeptide comprising in order from the N-terminus to C-terminus VL₁, CH1₁ and a first Fc domain subunit, a second polypeptide comprising in order from the N-terminus to C-terminus VH₁ and CL₁, and a third polypeptide comprising in order from the N-terminus to C-terminus a first cytokine receptor-binding domain and a second Fc domain subunit, and a second antigen binding molecule comprising a first polypeptide comprising in order from the N-terminus to C-terminus VH₂, CH1₂ and a first Fc domain subunit, a second polypeptide comprising in order from the N-terminus to C-terminus VL₂ and CL₂, and a third polypeptide comprising in order from the N-terminus to C-terminus a second cytokine receptor-binding domain and a second Fc domain subunit.

In one aspect, the pair of antigen binding molecules comprises first antigen binding molecule comprising a first polypeptide comprising an amino acid sequence of SEQ ID NO: 32, a second polypeptide comprising an amino acid sequence of SEQ ID NO: 25 and a third polypeptide comprising an amino acid sequence of SEQ ID NO: 39, and a second antigen binding molecule comprising a first polypeptide comprising an amino acid sequence of SEQ ID NO: 34, a second polypeptide comprising an amino acid sequence of SEQ ID NO: 29 and a third polypeptide comprising an amino acid sequence of SEQ ID NO: 40.

In one aspect, the pair of antigen binding molecules comprises first antigen binding molecule comprising a first polypeptide comprising an amino acid sequence of SEQ ID NO: 34, a second polypeptide comprising an amino acid sequence of SEQ ID NO: 29 and a third polypeptide comprising an amino acid sequence of SEQ ID NO: 39, and a second antigen binding molecule comprising a first polypeptide comprising an amino acid sequence of SEQ ID NO: 32, a second polypeptide comprising an amino acid sequence of SEQ ID NO: 25 and a third polypeptide comprising an amino acid sequence of SEQ ID NO: 40.

In one aspect, the pair of antigen binding molecules comprises first antigen binding molecule comprising a first polypeptide comprising an amino acid sequence of SEQ ID NO: 237, a second polypeptide comprising an amino acid sequence of SEQ ID NO: 32 and a third polypeptide comprising an amino acid sequence of SEQ ID NO: 25, and a second antigen binding molecule comprising a first polypeptide comprising an amino acid sequence of SEQ ID NO: 238, a second polypeptide comprising an amino acid sequence of SEQ ID NO: 34 and a third polypeptide comprising an amino acid sequence of SEQ ID NO: 29.

In one aspect, the pair of antigen binding molecules comprises first antigen binding molecule comprising a first polypeptide comprising an amino acid sequence of SEQ ID NO: 235, a second polypeptide comprising an amino acid sequence of SEQ ID NO: 32 and a third polypeptide comprising an amino acid sequence of SEQ ID NO: 25, and a second antigen binding molecule comprising a first polypeptide comprising an amino acid sequence of SEQ ID NO: 236, a second polypeptide comprising an amino acid sequence of SEQ ID NO: 34 and a third polypeptide comprising an amino acid sequence of SEQ ID NO: 29.

In one aspect, the pair of antigen binding molecules comprises first antigen binding molecule comprising a first polypeptide comprising an amino acid sequence of SEQ ID NO: 239, a second polypeptide comprising an amino acid sequence of SEQ ID NO: 32 and a third polypeptide comprising an amino acid sequence of SEQ ID NO: 25, and a second antigen binding molecule comprising a first polypeptide comprising an amino acid sequence of SEQ ID NO: 240, a second polypeptide comprising an amino acid sequence of SEQ ID NO: 34 and a third polypeptide comprising an amino acid sequence of SEQ ID NO: 29.

In one aspect, the pair of antigen binding molecules comprises first antigen binding molecule comprising a first polypeptide comprising an amino acid sequence of SEQ ID NO: 235, a second polypeptide comprising an amino acid sequence of SEQ ID NO: 32 and a third polypeptide comprising an amino acid sequence of SEQ ID NO: 25, and a second antigen binding molecule comprising a first polypeptide comprising an amino acid sequence of SEQ ID NO: 237, a second polypeptide comprising an amino acid sequence of SEQ ID NO: 34 and a third polypeptide comprising an amino acid sequence of SEQ ID NO: 29.

In one aspect, the pair of antigen binding molecules comprises first antigen binding molecule comprising a first polypeptide comprising an amino acid sequence of SEQ ID NO: 236, a second polypeptide comprising an amino acid sequence of SEQ ID NO: 32 and a third polypeptide comprising an amino acid sequence of SEQ ID NO: 25, and a second antigen binding molecule comprising a first polypeptide comprising an amino acid sequence of SEQ ID NO: 235, a second polypeptide comprising an amino acid sequence of SEQ ID NO: 34 and a third polypeptide comprising an amino acid sequence of SEQ ID NO: 29.

In one aspect, the pair of antigen binding molecules comprises first antigen binding molecule comprising a first polypeptide comprising an amino acid sequence of SEQ ID NO: 240, a second polypeptide comprising an amino acid sequence of SEQ ID NO: 32 and a third polypeptide comprising an amino acid sequence of SEQ ID NO: 25, and a second antigen binding molecule comprising a first polypeptide comprising an amino acid sequence of SEQ ID NO: 239, a second polypeptide comprising an amino acid sequence of SEQ ID NO: 34 and a third polypeptide comprising an amino acid sequence of SEQ ID NO: 29.

In one aspect, both antigen binding molecules of the pair of antigen binding molecules comprise a target-binding domain, a cytokine receptor-binding domain and a Fc domain, wherein the target-binding domain is fused at its C-terminus to the N-terminus of one of the Fc domain subunits and the cytokine receptor-binding domain is fused at its N-terminus to the C-terminus of the same Fc domain subunit.

Thus in one aspect, the pair of antigen binding molecules comprises a first antigen binding molecule comprising a first target-binding domain fused at its C-terminus to the N-terminus of one of the Fc domain subunits and a first cytokine receptor-binding domain fused at its N-terminus to the C-terminus of the same Fc domain subunit, and second antigen binding molecule comprising a second target-binding domain fused at its C-terminus to the N-terminus of one of the Fc domain subunits and a second cytokine receptor-binding domain fused at its N-terminus to the C-terminus of the same Fc domain subunit.

In one aspect, the target-binding domain is a Fab molecule. Thus in one embodiment, the pair of antigen binding molecules comprises a first antigen binding molecule comprising a target-binding domain fused at its C-terminus of CH1₁ to the N-terminus of one of the Fc domain subunits and a first cytokine receptor-binding domain is fused at its N-terminus to the C-terminus of the same Fc domain subunit, and a second antigen binding molecule comprising a target-binding domain fused at its C-terminus of CH1₂ to the N-terminus of one of the Fc domain subunits and a second cytokine receptor-binding domain fused at its N-terminus to the C-terminus of the same Fc domain subunit.

In one aspect, the target-binding domain of one of the antigen-binding molecules is a cross-Fab molecule. Thus, in one embodiment, the pair of antigen binding molecules comprises a first antigen binding molecule comprising a target-binding domain fused at its C-terminus of CH1₁ to the N-terminus of one of the Fc domain subunits and a first cytokine receptor-binding domain is fused at its N-terminus to the C-terminus of the same Fc domain subunit, and a second antigen binding molecule comprising a target-binding domain fused at its C-terminus of CL₂ to the N-terminus of one of the Fc domain subunits and a second cytokine receptor-binding domain fused at its N-terminus to the C-terminus of the same Fc domain subunit. In one embodiment, the pair of antigen binding molecules comprises a first antigen binding molecule comprising a target-binding domain fused at its C-terminus of CL₁ to the N-terminus of one of the Fc domain subunits and a first cytokine receptor-binding domain is fused at its N-terminus to the C-terminus of the same Fc domain subunit, and a second antigen binding molecule comprising a target-binding domain fused at its C-terminus of CH1₂ to the N-terminus of one of the Fc domain subunits and a second cytokine receptor-binding domain fused at its N-terminus to the C-terminus of the same Fc domain subunit.

In one aspect, the pair of antigen binding molecules comprises a first antigen binding molecule comprising a first polypeptide comprising a first Fc domain subunit, a second polypeptide comprising in order from the N-terminus to C-terminus a VH₁, a CH1₁, a second Fc domain subunit and a first cytokine receptor-binding domain, and a third polypeptide comprising in order from the N-terminus to C-terminus a VL₁ and a CL₁; and a second antigen binding molecule comprising a first polypeptide comprising a first Fc domain subunit, a second polypeptide comprising in order from the N-terminus to C-terminus a VH₂, a CH1₂, a second Fc domain subunit and a second cytokine receptor-binding domain, and a third polypeptide comprising in order from the N-terminus to C-terminus VL₂ and CL₂.

In one aspect, the pair of antigen binding molecules comprises a first antigen binding molecule comprising a first polypeptide comprising a first polypeptide comprising a first Fc domain subunit, a second polypeptide comprising in order from the N-terminus to C-terminus a VL₁, a CH1₁, a second Fc domain subunit and a first cytokine receptor-binding domain, and a third polypeptide comprising in order from the N-terminus to C-terminus a VH₁ and a CL₁; and a second antigen binding molecule comprising a first polypeptide comprising a first Fc domain subunit, a second polypeptide comprising in order from the N-terminus to C-terminus a VH₂, a CH1₂, a second Fc domain subunit and a second cytokine receptor-binding domain, and a third polypeptide comprising in order from the N-terminus to C-terminus a VL₂ and a CL₂.

In one aspect, the pair of antigen binding molecules comprises first antigen binding molecule comprising a first polypeptide comprising an amino acid sequence of SEQ ID NO: 28, a second polypeptide comprising an amino acid sequence of SEQ ID NO: 42 and a third polypeptide comprising an amino acid sequence of SEQ ID NO: 41, and a second antigen binding molecule comprising a first polypeptide comprising an amino acid sequence of SEQ ID NO: 45, a second polypeptide comprising an amino acid sequence of SEQ ID NO: 44 and a third polypeptide comprising an amino acid sequence of SEQ ID NO: 43.

In one aspect, the pair of antigen binding molecules comprises first antigen binding molecule comprising a first polypeptide comprising an amino acid sequence of SEQ ID NO: 28, a second polypeptide comprising an amino acid sequence of SEQ ID NO: 46 and a third polypeptide comprising an amino acid sequence of SEQ ID NO: 41, and a second antigen binding molecule comprising a first polypeptide comprising an amino acid sequence of SEQ ID NO: 47, a second polypeptide comprising an amino acid sequence of SEQ ID NO: 44 and a third polypeptide comprising an amino acid sequence of SEQ ID NO: 43.

In one aspect, the pair of antigen binding molecules comprises first antigen binding molecule comprising a first polypeptide comprising an amino acid sequence of SEQ ID NO: 241, a second polypeptide comprising an amino acid sequence of SEQ ID NO: 246 and a third polypeptide comprising an amino acid sequence of SEQ ID NO: 25, and a second antigen binding molecule comprising a first polypeptide comprising an amino acid sequence of SEQ ID NO: 241, a second polypeptide comprising an amino acid sequence of SEQ ID NO: 249 and a third polypeptide comprising an amino acid sequence of SEQ ID NO: 29.

In one aspect, the pair of antigen binding molecules comprises first antigen binding molecule comprising a first polypeptide comprising an amino acid sequence of SEQ ID NO: 241, a second polypeptide comprising an amino acid sequence of SEQ ID NO: 242 and a third polypeptide comprising an amino acid sequence of SEQ ID NO: 25, and a second antigen binding molecule comprising a first polypeptide comprising an amino acid sequence of SEQ ID NO: 241, a second polypeptide comprising an amino acid sequence of SEQ ID NO: 245 and a third polypeptide comprising an amino acid sequence of SEQ ID NO: 29.

In one aspect, the pair of antigen binding molecules comprises first antigen binding molecule comprising a first polypeptide comprising an amino acid sequence of SEQ ID NO: 241, a second polypeptide comprising an amino acid sequence of SEQ ID NO: 250 and a third polypeptide comprising an amino acid sequence of SEQ ID NO: 25, and a second antigen binding molecule comprising a first polypeptide comprising an amino acid sequence of SEQ ID NO: 241, a second polypeptide comprising an amino acid sequence of SEQ ID NO: 253 and a third polypeptide comprising an amino acid sequence of SEQ ID NO: 29.

In one aspect, the pair of antigen binding molecules comprises first antigen binding molecule comprising a first polypeptide comprising an amino acid sequence of SEQ ID NO: 241, a second polypeptide comprising an amino acid sequence of SEQ ID NO: 248 and a third polypeptide comprising an amino acid sequence of SEQ ID NO: 25, and a second antigen binding molecule comprising a first polypeptide comprising an amino acid sequence of SEQ ID NO: 241, a second polypeptide comprising an amino acid sequence of SEQ ID NO: 247 and a third polypeptide comprising an amino acid sequence of SEQ ID NO: 29.

In one aspect, the pair of antigen binding molecules comprises first antigen binding molecule comprising a first polypeptide comprising an amino acid sequence of SEQ ID NO: 241, a second polypeptide comprising an amino acid sequence of SEQ ID NO: 244 and a third polypeptide comprising an amino acid sequence of SEQ ID NO: 25, and a second antigen binding molecule comprising a first polypeptide comprising an amino acid sequence of SEQ ID NO: 241, a second polypeptide comprising an amino acid sequence of SEQ ID NO: 243 and a third polypeptide comprising an amino acid sequence of SEQ ID NO: 29.

In one aspect, the pair of antigen binding molecules comprises first antigen binding molecule comprising a first polypeptide comprising an amino acid sequence of SEQ ID NO: 241, a second polypeptide comprising an amino acid sequence of SEQ ID NO: 252 and a third polypeptide comprising an amino acid sequence of SEQ ID NO: 25, and a second antigen binding molecule comprising a first polypeptide comprising an amino acid sequence of SEQ ID NO: 241, a second polypeptide comprising an amino acid sequence of SEQ ID NO: 251 and a third polypeptide comprising an amino acid sequence of SEQ ID NO: 29.

In one aspect, the pair of antigen binding molecules comprises first antigen binding molecule comprising a first polypeptide comprising an amino acid sequence of SEQ ID NO: 28, a second polypeptide comprising an amino acid sequence of SEQ ID NO: 42 and a third polypeptide comprising an amino acid sequence of SEQ ID NO: 42, and a second antigen binding molecule comprising a first polypeptide comprising an amino acid sequence of SEQ ID NO: 241, a second polypeptide comprising an amino acid sequence of SEQ ID NO: 245 and a third polypeptide comprising an amino acid sequence of SEQ ID NO: 29.

### Alternative Configurations of the Antigen Binding Molecules

The antigen binding molecule(s) may comprise a target-binding domain, a cytokine receptor-binding domain and a Fc domain, wherein the cytokine receptor-binding domain is fused at its C-terminus to the N-terminus of the target-binding domain, and the target-binding domain is fused at its C-terminus to the N-terminus of one of the Fc domain subunits, wherein the target-binding domain is a VHH domain. Thus, the antigen binding molecule may comprise a first polypeptide comprising a first Fc domain subunit and a second polypeptide comprising in order from the N-terminus to C-terminus a first cytokine receptor-binding domain, a first target-binding domain and a second Fc domain subunit, and a second antigen binding molecule comprising a first polypeptide comprising a first Fc domain subunit and a second polypeptide comprising in order from the N-terminus to C-terminus a second cytokine receptor-binding domain, a second target-binding domain and a second Fc domain subunit.

In one aspect, the pair of antigen binding molecules comprises first antigen binding molecule comprising a first polypeptide comprising an amino acid sequence of SEQ ID NO: 28 and a second polypeptide comprising an amino acid sequence of SEQ ID NO: 173, and a second antigen binding molecule comprising a first polypeptide comprising an amino acid sequence of SEQ ID NO: 28 and a second polypeptide comprising an amino acid sequence of SEQ ID NO: 171.

In one aspect, the pair of antigen binding molecules comprises first antigen binding molecule comprising a first polypeptide comprising an amino acid sequence of SEQ ID NO: 28 and a second polypeptide comprising an amino acid sequence of SEQ ID NO: 176, and a second antigen binding molecule comprising a first polypeptide comprising an amino acid sequence of SEQ ID NO: 28 and a second polypeptide comprising an amino acid sequence of SEQ ID NO: 172.

In one aspect, the pair of antigen binding molecules comprises first antigen binding molecule comprising a first polypeptide comprising an amino acid sequence of SEQ ID NO: 28 and a second polypeptide comprising an amino acid sequence of SEQ ID NO: 175, and a second antigen binding molecule comprising a first polypeptide comprising an amino acid sequence of SEQ ID NO: 28 and a second polypeptide comprising an amino acid sequence of SEQ ID NO: 171.

In one aspect, the pair of antigen binding molecules comprises first antigen binding molecule comprising a first polypeptide comprising an amino acid sequence of SEQ ID NO: 28 and a second polypeptide comprising an amino acid sequence of SEQ ID NO: 176, and a second antigen binding molecule comprising a first polypeptide comprising an amino acid sequence of SEQ ID NO: 28 and a second polypeptide comprising an amino acid sequence of SEQ ID NO: 174.

In one aspect, the pair of antigen binding molecules comprises first antigen binding molecule comprising a first polypeptide comprising an amino acid sequence of SEQ ID NO: 28 and a second polypeptide comprising an amino acid sequence of SEQ ID NO: 175, and a second antigen binding molecule comprising a first polypeptide comprising an amino acid sequence of SEQ ID NO: 28 and a second polypeptide comprising an amino acid sequence of SEQ ID NO: 170.

In one aspect, the pair of antigen binding molecules comprises first antigen binding molecule comprising a first polypeptide comprising an amino acid sequence of SEQ ID NO: 28 and a second polypeptide comprising an amino acid sequence of SEQ ID NO: 173, and a second antigen binding molecule comprising a first polypeptide comprising an amino acid sequence of SEQ ID NO: 28 and a second polypeptide comprising an amino acid sequence of SEQ ID NO: 170.

In one aspect, the pair of antigen binding molecules comprises first antigen binding molecule comprising a first polypeptide comprising an amino acid sequence of SEQ ID NO: 28 and a second polypeptide comprising an amino acid sequence of SEQ ID NO: 176, and a second antigen binding molecule comprising a first polypeptide comprising an amino acid sequence of SEQ ID NO: 28 and a second polypeptide comprising an amino acid sequence of SEQ ID NO: 172.

The antigen binding molecule(s) may comprise a target-binding domain, a cytokine receptor-binding domain and a Fc domain, wherein the cytokine receptor-binding domain is fused at its C-terminus to the N-terminus of the target-binding domain, and the target-binding domain is fused at its C-terminus to the N-terminus of one of the Fc domain subunits, wherein the cytokine receptor-binding domain is a Fab molecule.

In one aspect, the pair of antigen binding molecules comprises first antigen binding molecule comprising a first polypeptide comprising an amino acid sequence of SEQ ID NO: 28, a second polypeptide comprising an amino acid sequence of SEQ ID NO: 267, a third polypeptide comprising an amino acid sequence of SEQ ID NO: 268 and a fourth polypeptide comprising an amino acid sequence of SEQ ID NO: 265, and a second antigen binding molecule comprising a first polypeptide comprising an amino acid sequence of SEQ ID NO: 28, a second polypeptide comprising an amino acid sequence of SEQ ID NO: 272, a third polypeptide comprising an amino acid sequence of SEQ ID NO: 270 and a fourth polypeptide comprising an amino acid sequence of SEQ ID NO: 271.

In one aspect, the pair of antigen binding molecules comprises first antigen binding molecule comprising a first polypeptide comprising an amino acid sequence of SEQ ID NO: 28, a second polypeptide comprising an amino acid sequence of SEQ ID NO: 267, a third polypeptide comprising an amino acid sequence of SEQ ID NO: 268 and a fourth polypeptide comprising an amino acid sequence of SEQ ID NO: 265, and a second antigen binding molecule comprising a first polypeptide comprising an amino acid sequence of SEQ ID NO: 28, a second polypeptide comprising an amino acid sequence of SEQ ID NO: 274, a third polypeptide comprising an amino acid sequence of SEQ ID NO: 270 and a fourth polypeptide comprising an amino acid sequence of SEQ ID NO: 273.

In one aspect, the pair of antigen binding molecules comprises first antigen binding molecule comprising a first polypeptide comprising an amino acid sequence of SEQ ID NO: 28, a second polypeptide comprising an amino acid sequence of SEQ ID NO: 269, a third polypeptide comprising an amino acid sequence of SEQ ID NO: 265 and a fourth polypeptide comprising an amino acid sequence of SEQ ID NO: 268, and a second antigen binding molecule comprising a first polypeptide comprising an amino acid sequence of SEQ ID NO: 28, a second polypeptide comprising an amino acid sequence of SEQ ID NO: 272, a third polypeptide comprising an amino acid sequence of SEQ ID NO: 270 and a fourth polypeptide comprising an amino acid sequence of SEQ ID NO: 271.

In one aspect, the pair of antigen binding molecules comprises first antigen binding molecule comprising a first polypeptide comprising an amino acid sequence of SEQ ID NO: 28, a second polypeptide comprising an amino acid sequence of SEQ ID NO: 269, a third polypeptide comprising an amino acid sequence of SEQ ID NO: 265 and a fourth polypeptide comprising an amino acid sequence of SEQ ID NO: 268, and a second antigen binding molecule comprising a first polypeptide comprising an amino acid sequence of SEQ ID NO: 28, a second polypeptide comprising an amino acid sequence of SEQ ID NO: 274, a third polypeptide comprising an amino acid sequence of SEQ ID NO: 270 and a fourth polypeptide comprising an amino acid sequence of SEQ ID NO: 273.

The antigen binding molecule(s) may comprise a target-binding domain, a cytokine receptor-binding domain and a Fc domain, wherein the cytokine receptor-binding domain is fused at its C-terminus to the N-terminus of the target-binding domain, and the target-binding domain is fused at its C-terminus to the N-terminus of one of the Fc domain subunits, wherein the cytokine receptor-binding domain is a scFv domain.

In one aspect, the pair of antigen binding molecules comprises first antigen binding molecule comprising a first polypeptide comprising an amino acid sequence of SEQ ID NO: 28, a second polypeptide comprising an amino acid sequence of SEQ ID NO: 276 and a third polypeptide comprising an amino acid sequence of SEQ ID NO: 275, and a second antigen binding molecule comprising a first polypeptide comprising an amino acid sequence of SEQ ID NO: 28, a second polypeptide comprising an amino acid sequence of SEQ ID NO: 277 and a third polypeptide comprising an amino acid sequence of SEQ ID NO: 278.

### SPECIFIC EMBODIMENTS OF THE INVENTION

In the following specific embodiments of the invention are listed.

1. A pair of antigen binding molecules that specifically bind to a target antigen, comprising a) a first antigen binding molecule comprising i) a first target-binding domain, ii) a first cytokine receptor-binding domain, and iii) a Fc domain; and b) a second antigen binding molecule comprising i) a second target-binding domain, ii) a second cytokine receptor-binding domain, and iii) a Fc domain; wherein the first target-binding domain is capable of binding a first epitope on the target antigen and the second target-binding domain is capable of binding a second epitope on the target antigen, wherein the first and second target-binding domains do not compete for binding on the target antigen; and wherein the first cytokine receptor-binding domain is capable of binding a first cytokine receptor subunit and the second cytokine receptor-binding domain is capable of binding a second cytokine receptor subunit.

2. The pair of antigen binding molecules according to embodiment 1, wherein the first and second target-binding domains are antibody fragments, such as Fv, Fab, scFv, scFab molecules or single domain antibodies.

3. The pair of antigen binding molecules according to embodiment 1 or 2, wherein the first and second target-binding domains are Fab molecules.

4. The pair of antigen binding molecules according to embodiment 3, wherein the first target-binding domain comprises a heavy chain variable domain (VH₁), a light chain variable domain (VL₁), a heavy chain constant domain (CH1₁) and a light chain constant domain (CL₁), and wherein the second target-binding domain comprises a heavy chain variable domain (VH₂), a light chain variable domain (VL₂), a heavy chain constant domain (CH1₂) and a light chain constant domain (CL₂).

5. The pair of antigen binding molecules according to embodiment 3 or 4, wherein the first and/or second target-binding domain is a cross-Fab molecule.

6. The pair of antigen binding molecules according to any one of the preceding embodiments, wherein the first and second target-binding domains specifically bind to a tumor-associated antigen or a T-cell antigen.

7. The pair of antigen binding molecules according to any one of the preceding embodiments, wherein the first and second target-binding domains specifically bind to FAP, PD-1, Her2, Her3, LAG-3 or EGFR.

8. The pair of antigen binding molecules according to any one of the preceding embodiments, wherein a) the first target-binding domain comprises a VH₁ of SEQ ID NO: 20 and a VL₁ of SEQ ID NO: 21 and the second target-binding domain comprises a VH₂ of SEQ ID NO: 22 and a VL₂ of SEQ ID NO: 23, or b) the first target-binding domain comprises a VH₁ of SEQ ID NO: 22 and a VL₁ of SEQ ID NO: 23 and the second target-binding domain comprises a VH₂ of SEQ ID NO: 20 and a VL₂ of SEQ ID NO: 21, or c) the first target-binding domain comprises a VH₁ of SEQ ID NO: 76 and a VL₁ of SEQ ID NO: 77 and the second target-binding domain comprises a VH₂ of SEQ ID NO: 78 and a VL₂ of SEQ ID NO: 79, or d) the first target-binding domain comprises a VH₁ of SEQ ID NO: 78 and a VL₁ of SEQ ID NO: 79 and the second target-binding domain comprises a VH₂ of SEQ ID NO: 76 and a VL₂ of SEQ ID NO: 77; or e) the first target-binding domain comprises a VH₁ of SEQ ID NO: 93 and a VL₁ of SEQ ID NO: 94 and the second target-binding domain comprises a VH₂ of SEQ ID NO: 78 and a VL₂ of SEQ ID NO: 79; or f) the first target-binding domain comprises a VH₁ of SEQ ID NO: 78 and a VL₁ of SEQ ID NO: 79 and the second target-binding domains comprises a VH₂ of SEQ ID NO: 93 and a VL₂ of SEQ ID NO: 94, or g) the first target-binding domain comprises a VH₁ of SEQ ID NO: 130 and a VL₁ of SEQ ID NO: 131 and the second target-binding domain comprises a VH₂ of SEQ ID NO: 132 and a VL₂ of SEQ ID NO: 133; or h) the first target-binding domain comprises a VH₁ of SEQ ID NO: 132 and a VL₁ of SEQ ID NO: 133 and the second target-binding domains comprises a VH₂ of SEQ ID NO: 130 and a VL₂ of SEQ ID NO: 131; or i) the first target-binding domain comprises a VH₁ of SEQ ID NO: 140 and a VL₁ of SEQ ID NO: 141 and the second target-binding domain comprises a VH₂ of SEQ ID NO: 142 and a VL₂ of SEQ ID NO: 143; or j) the first target-binding domain comprises a VH₁ of SEQ ID NO: 142 and a VL₁ of SEQ ID NO: 143 and the second target-binding domains comprises a VH₂ of SEQ ID NO: 140 and a VL₂ of SEQ ID NO: 141; or k) the first target-binding domain comprises a VH₁ of SEQ ID NO: 114 and a VL₁ of SEQ ID NO: 115 and the second target-binding domain comprises a VH₂ of SEQ ID NO: 116 and a VL₂ of SEQ ID NO: 117; or l) the first target-binding domain comprises a VH₁ of SEQ ID NO: 116 and a VL₁ of SEQ ID NO: 117 and the second target-binding domains comprises a VH₂ of SEQ ID NO: 114 and a VL₂ of SEQ ID NO: 115; or m) the first target-binding domain comprises a VH₁ of SEQ ID NO: 150 and a VL₁ of SEQ ID NO: 151 and the second target-binding domain comprises a VH₂ of SEQ ID NO: 152 and a VL₂ of SEQ ID NO: 153; or n) the first target-binding domain comprises a VH₁ of SEQ ID NO: 152 and a VL₁ of SEQ ID NO: 153 and the second target-binding domains comprises a VH₂ of SEQ ID NO: 150 and a VL₂ of SEQ ID NO: 151.

9. The pair of antigen binding molecules according to any one of the preceding embodiments, wherein both the first and second cytokine receptor subunits are subunits of the IFNγ receptor complex or, IL-2 receptor complex or IL-7 receptor complex.

10. The pair of antigen binding molecules according to any one of the preceding embodiments, wherein a) the first cytokine receptor-binding domain is capable of binding IFNγR1 and the second cytokine receptor-binding domain is capable of binding IFNγR2, or b) the first cytokine receptor-binding domain is capable of binding IFNγR2 and the second cytokine receptor-binding domain is capable of binding IFNγR1; or c) the first cytokine receptor-binding domain is capable of binding IL-2Rβ and the second cytokine receptor-binding domain is capable of binding IL-2Rγ, or d) the first cytokine receptor-binding domain is capable of binding IL-2Rγ and the second cytokine receptor-binding domain is capable of binding IL-2Rβ; or e) the first cytokine receptor-binding domain is capable of binding IL-2Rγ and the second cytokine receptor-binding domain is capable of binding IL-7Rα; or f) the first cytokine receptor-binding domain is capable of binding IL-7Rα and the second cytokine receptor-binding domain is capable of binding IL-2Rγ.

11. The pair of antigen binding molecules according to any one of the preceding embodiments, wherein the first and second cytokine receptor-binding domains are antibody fragments, such as Fv, Fab, scFv, scFab or single domain antibodies.

12. The pair of antigen binding molecules according to any one of the preceding embodiments, wherein the first and second cytokine receptor-binding domains are VHH domains.

13. The pair of antigen binding molecules according to any one of the preceding embodiments, wherein a) the first cytokine receptor-binding domain comprises an amino acid sequence selected from SEQ ID NO: 1, SEQ ID NO: 2, SEQ ID NO: 3 and SEQ ID NO: 5 and the second cytokine receptor-binding domain comprises an amino acid sequence selected from SEQ ID NO: 6, SEQ ID NO: 7, SEQ ID NO: 8, SEQ ID NO: 9 and SEQ ID NO: 10, or b) the first cytokine receptor-binding domain comprises an amino acid sequence selected from SEQ ID NO: 6, SEQ ID NO: 7, SEQ ID NO: 8, SEQ ID NO: 9 and SEQ ID NO: 10 and the second cytokine receptor-binding domain comprises an amino acid sequence selected from SEQ ID NO: 1, SEQ ID NO: 2, SEQ ID NO: 3 and SEQ ID NO: 5; or c) the first cytokine receptor-binding domain comprises an amino acid sequence selected from SEQ ID NO: 56, SEQ ID NO: 57, SEQ ID NO: 58 SEQ ID NO: 60, and the second cytokine receptor-binding domain comprises an amino acid sequence selected from SEQ ID NO: 62, SEQ ID NO: 63, SEQ ID NO: 64 and SEQ ID NO: 65; or d) the first cytokine receptor-binding domain comprises an amino acid sequence selected from SEQ ID NO: 62, SEQ ID NO: 63, SEQ ID NO: 64 and SEQ ID NO: 65 and the second cytokine receptor-binding domain comprises an amino acid sequence selected from SEQ ID NO: 56, SEQ ID NO: 57, SEQ ID NO: 58 SEQ ID NO: 60; or e) the first cytokine receptor-binding domain comprises an amino acid sequence selected from SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, and the second cytokine receptor-binding domain comprises an amino acid sequence selected from SEQ ID NO: 61, SEQ ID NO: 62, SEQ ID NO: 63, SEQ ID NO: 64, SEQ ID NO: 65; or f) the first cytokine receptor-binding domain comprises an amino acid sequence selected from SEQ ID NO: 61, SEQ ID NO: 62, SEQ ID NO: 63, SEQ ID NO: 64, SEQ ID NO: 65, and the second cytokine receptor-binding domain comprises an amino acid sequence selected from SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192.

14. The pair of antigen binding molecules according to any one of the preceding embodiments, wherein a) the first cytokine receptor-binding domain comprises the sequence of SEQ ID NO: 3 and the second cytokine receptor-binding domain comprises the sequence of SEQ ID NO: 7, or b) the first cytokine receptor-binding domain comprises the sequence of SEQ ID NO: 7 and the second cytokine receptor-binding domain comprises the sequence of SEQ ID NO: 3; or c) the first cytokine receptor-binding domain comprises the sequence of SEQ ID NO: 60 and the second cytokine receptor-binding domain comprises the sequence of SEQ ID NO: 62, or d) the first cytokine receptor-binding domain comprises the sequence of SEQ ID NO: 62 and the second cytokine receptor-binding domain comprises the sequence of SEQ ID NO: 60; or e) the first cytokine receptor-binding domain comprises the sequence of SEQ ID NO: 186, and the second cytokine receptor-binding domain comprises the sequence of SEQ ID NO: 62, or f) the first cytokine receptor-binding domain comprises the sequence of SEQ ID NO: 62, and the second cytokine receptor-binding domain comprises the sequence of SEQ ID NO: 186.

15. The pair of antigen binding molecules according to any one of the preceding embodiments, wherein the Fc domains of the first and second antigen binding molecules comprise a first Fc domain subunit and a second Fc domain subunit.

16. The pair of antigen binding molecules according to any one of the preceding embodiments, wherein the Fc domains of the first and second antigen binding molecules are IgG, particularly an IgG₁, Fc domains.

17. The pair of antigen binding molecules according to any one of the preceding embodiments, wherein the Fc domains of the first and second antigen binding molecules are human Fc domains.

18. The pair of antigen binding molecules according to any one of the preceding embodiments, wherein the Fc domains of the first and second antigen binding molecules comprise a modification promoting the association of the first and the second subunit of the Fc domains.

19. The pair of antigen binding molecules according to any one of the preceding embodiments, wherein the Fc domains of the first and second antigen binding molecules comprise one or more amino acid substitution that reduces binding to an Fc receptor and/or effector function.

20. The pair of antigen binding molecules according to any one of the preceding embodiments, wherein the cytokine receptor-binding domains are fused via peptide linkers to their respective fusion points.

21. The pair of antigen binding molecules according to embodiments 20, wherein the peptide linkers comprise an amino acid sequence selected from SEQ ID NO: 262, SEQ ID NO: 263, SEQ ID NO: 264, SEQ ID NO: 279, SEQ ID NO: 280 or SEQ ID NO: 281.

22. The pair of antigen binding molecules according to any one of the preceding embodiments, wherein the first cytokine receptor-binding domain is fused at its C-terminus to the N-terminus of VH₁ or VL₁ of the first target-binding domain and the second cytokine receptor-binding domain is fused at its C-terminus to the N-terminus of VH₂ or VL₂ of the second target-binding domain.

23. The pair of antigen binding molecules according to any one of the preceding embodiments, wherein a) the first antigen binding molecule comprises a first polypeptide comprising a first Fc domain subunit, a second polypeptide comprising in order from the N-terminus to C-terminus the first cytokine receptor-binding domain, VH₁, CH1₁ and a second Fc domain subunit, and a third polypeptide comprising in order from the N-terminus to C-terminus VL₁ and CL₁; and the second antigen binding molecule comprises a first polypeptide comprising a first Fc domain subunit, a second polypeptide comprising in order from the N-terminus to C-terminus the second cytokine receptor-binding domain, VH₂, CH1₂ and a second Fc domain subunit, and a third polypeptide comprising in order from the N-terminus to C-terminus VL₂ and CL₂; or b) the first antigen binding molecule comprises a first polypeptide comprising a first Fc domain subunit, a second polypeptide comprising in order from the N-terminus to C-terminus VH₁, CH1₁ and a second Fc domain subunit, and a third polypeptide comprising in order from the N-terminus to C-terminus the first cytokine receptor-binding domain, VL₁ and CL₁; and the second antigen binding molecule comprises a first polypeptide comprising a first Fc domain subunit, a second polypeptide comprising in order from the N-terminus to C-terminus VH₂, CH1₂ and a second Fc domain subunit, a third polypeptide comprising in order from the N-terminus to C-terminus the second cytokine receptor-binding domain, VL₂ and CL₂; or c) the first antigen binding molecule comprises a first polypeptide comprising a first Fc domain subunit, a second polypeptide comprising in order from the N-terminus to C-terminus the first cytokine receptor-binding domain, VH₁, CH1₁ and a second Fc domain subunit, and a third polypeptide comprising in order from the N-terminus to C-terminus VL₁ and CL₁; and the second antigen binding molecule comprises a first polypeptide comprising a first Fc domain subunit, a second polypeptide comprising in order from the N-terminus to C-terminus VH₂, CH1₂ and a second Fc domain subunit, a third polypeptide comprising in order from the N-terminus to C-terminus the second cytokine receptor-binding domain, VL₂ and CL₂; wherein VH₁, VL₁, CH1₁ and CL₁ form the first target-binding domain and VH₂, VL₂, CH1₂, and CL₂, form the second target-binding domain.

24. The pair of antigen binding molecules according to any one of the preceding embodiments, wherein a) the first antigen binding molecule comprises a first polypeptide comprising a first Fc domain subunit, a second polypeptide comprising in order from the N-terminus to C-terminus the first cytokine receptor-binding domain, VH₁, CH1₁ and a second Fc domain subunit, and a third polypeptide comprising in order from the N-terminus to C-terminus VL₁ and CL₁, and the second antigen binding molecule comprises a first polypeptide comprising a first Fc domain subunit, a second polypeptide comprising in order from the N-terminus to C-terminus the second cytokine receptor-binding domain, VH₂, CH1₂ and a second Fc domain subunit, and a third polypeptide comprising in order from the N-terminus to C-terminus VL₂ and CL₂; or b) the first antigen binding molecule comprises a first polypeptide comprising a first Fc domain subunit, a second polypeptide comprising in order from the N-terminus to C-terminus VH₁, CH1₁ and a second Fc domain subunit, and a third polypeptide comprising in order from the N-terminus to C-terminus the first cytokine receptor-binding domain, VL₁ and CL₁, and the second antigen binding molecule comprises a first polypeptide comprising a first Fc domain subunit, a second polypeptide comprising in order from the N-terminus to C-terminus VH₂, CH1₂ and a second Fc domain subunit, a third polypeptide comprising in order from the N-terminus to C-terminus the second cytokine receptor-binding domain, VL₂ and CL₂; or c) the first antigen binding molecule comprises a first polypeptide comprising a first Fc domain subunit, a second polypeptide comprising in order from the N-terminus to C-terminus the first cytokine receptor-binding domain, VH₁, CH1₁ and a second Fc domain subunit, and a third polypeptide comprising in order from the N-terminus to C-terminus VL₁ and CL₁, and the second antigen binding molecule comprises a first polypeptide comprising a first Fc domain subunit, a second polypeptide comprising in order from the N-terminus to C-terminus VH₂, CH1₂ and a second Fc domain subunit, a third polypeptide comprising in order from the N-terminus to C-terminus the second cytokine receptor-binding domain, VL₂ and CL₂; or d) the first antigen binding molecule comprises a first polypeptide comprising a first Fc domain subunit, a second polypeptide comprising in order from the N-terminus to C-terminus the first cytokine receptor-binding domain, VL₁, CH1₁ and a second Fc domain subunit, and a third polypeptide comprising in order from the N-terminus to C-terminus VH₁ and CL₁, and the second antigen binding molecule comprises a first polypeptide comprising a first Fc domain subunit, a second polypeptide comprising in order from the N-terminus to C-terminus the second cytokine receptor-binding domain, VH₂, CH1₂ and a second Fc domain subunit, and a third polypeptide comprising in order from the N-terminus to C-terminus VL₂ and CL₂; or e) the first antigen binding molecule comprises a first polypeptide comprising a first Fc domain subunit, a second polypeptide comprising in order from the N-terminus to C-terminus VL₁, CH1₁ and a second Fc domain subunit, and a third polypeptide comprising in order from the N-terminus to C-terminus the first cytokine receptor-binding domain, VH₁ and CL₁, and the second antigen binding molecule comprises a first polypeptide comprising a first Fc domain subunit, a second polypeptide comprising in order from the N-terminus to C-terminus VH₂, CH1₂ and a second Fc domain subunit, a third polypeptide comprising in order from the N-terminus to C-terminus the second cytokine receptor-binding domain, VL₂ and CL₂; or f) the first antigen binding molecule comprises a first polypeptide comprising a first Fc domain subunit, a second polypeptide comprising in order from the N-terminus to C-terminus the first cytokine receptor-binding domain, VL₁, CH1₁ and a second Fc domain subunit, and a third polypeptide comprising in order from the N-terminus to C-terminus VH₁ and CL₁, and the second antigen binding molecule comprises a first polypeptide comprising a first Fc domain subunit, a second polypeptide comprising in order from the N-terminus to C-terminus VH₂, CH1₂ and a second Fc domain subunit, and a third polypeptide comprising in order from the N-terminus to C-terminus the second cytokine receptor-binding domain, VL₂ and CL₂; or g) the first antigen binding molecule comprising a first polypeptide comprising a first Fc domain subunit, a second polypeptide comprising in order from the N-terminus to C-terminus a VL₁, CH1₁ and a second Fc domain subunit, and a third polypeptide comprising in order from the N-terminus to C-terminus a first cytokine receptor-binding domain, VH₁ and CL₁, and the second antigen binding molecule comprises a first polypeptide comprising a first Fc domain subunit, a second polypeptide comprising in order from the N-terminus to C-terminus a second cytokine receptor-binding domain, VH₂, CH1₂ and a second Fc domain subunit, and a third polypeptide comprising in order from the N-terminus to C-terminus VL₂ and CL₂; wherein VH₁, VL₁, CH1₁ and CL₁ form the first target-binding domain and VH₂, VL₂, CH1₂, and CL₂, form the second target-binding domain.

25. The pair of antigen binding molecules according to any one of the preceding embodiments, wherein the first and the second target-binding domains specifically bind to FAP, and the first and second cytokine receptor subunits are subunits of the IFNγ receptor complex.

26. The pair of antigen binding molecules according to any one of the preceding embodiments, wherein a) the first antigen binding molecule comprises a first polypeptide comprising an amino acid sequence of SEQ ID NO: 28, a second polypeptide comprising an amino acid sequence of SEQ ID NO: 27 and a third polypeptide comprising an amino acid sequence of SEQ ID NO: 25, and the second antigen binding molecule comprises a first polypeptide comprising an amino acid sequence of SEQ ID NO: 28, a second polypeptide comprising an amino acid sequence of SEQ ID NO: 30 and a third polypeptide comprising an amino acid sequence of SEQ ID NO: 29; or b) the first antigen binding molecule comprises a first polypeptide comprising an amino acid sequence of SEQ ID NO: 28, a second polypeptide comprising an amino acid sequence of SEQ ID NO: 38 and a third polypeptide comprising an amino acid sequence of SEQ ID NO: 25, and the second antigen binding molecule comprises a first polypeptide comprising an amino acid sequence of SEQ ID NO: 28, a second polypeptide comprising an amino acid sequence of SEQ ID NO: 37 and a third polypeptide comprising an amino acid sequence of SEQ ID NO: 29; or c) the first antigen binding molecule comprises a first polypeptide comprising an amino acid sequence of SEQ ID NO: 28, a second polypeptide comprising an amino acid sequence of SEQ ID NO: 32 and a third polypeptide comprising an amino acid sequence of SEQ ID NO: 31, and the second antigen binding molecule comprises a first polypeptide comprising an amino acid sequence of SEQ ID NO: 28, a second polypeptide comprising an amino acid sequence of SEQ ID NO: 34 and a third polypeptide comprising an amino acid sequence of SEQ ID NO: 33; or d) the first antigen binding molecule comprises a first polypeptide comprising an amino acid sequence of SEQ ID NO: 28, a second polypeptide comprising an amino acid sequence of SEQ ID NO: 32 and a third polypeptide comprising an amino acid sequence of SEQ ID NO: 35, and the second antigen binding molecule comprises a first polypeptide comprising an amino acid sequence of SEQ ID NO: 28, a second polypeptide comprising an amino acid sequence of SEQ ID NO: 34 and a third polypeptide comprising an amino acid sequence of SEQ ID NO: 36; or e) the first antigen binding molecule comprises a first polypeptide comprising an amino acid sequence of SEQ ID NO: 28, a second polypeptide comprising an amino acid sequence of SEQ ID NO: 27 and a third polypeptide comprising an amino acid sequence of SEQ ID NO: 25, and the second antigen binding molecule comprises a first polypeptide comprising an amino acid sequence of SEQ ID NO: 28, a second polypeptide comprising an amino acid sequence of SEQ ID NO: 34 and a third polypeptide comprising an amino acid sequence of SEQ ID NO: 33; or f) the first antigen binding molecule comprises a first polypeptide comprising an amino acid sequence of SEQ ID NO: 28, a second polypeptide comprising an amino acid sequence of SEQ ID NO: 30 and a third polypeptide comprising an amino acid sequence of SEQ ID NO: 29, and the second antigen binding molecule comprises a first polypeptide comprising an amino acid sequence of SEQ ID NO: 28, a second polypeptide comprising an amino acid sequence of SEQ ID NO: 32 and a third polypeptide comprising an amino acid sequence of SEQ ID NO: 31; or g) the first antigen binding molecule comprising a first polypeptide comprises an amino acid sequence of SEQ ID NO: 28, a second polypeptide comprising an amino acid sequence of SEQ ID NO: 38 and a third polypeptide comprising an amino acid sequence of SEQ ID NO: 25, and the second antigen binding molecule comprises a first polypeptide comprising an amino acid sequence of SEQ ID NO: 28, a second polypeptide comprising an amino acid sequence of SEQ ID NO: 34 and a third polypeptide comprising an amino acid sequence of SEQ ID NO: 36; or h) the first antigen binding molecule comprises a first polypeptide comprising an amino acid sequence of SEQ ID NO: 28, a second polypeptide comprising an amino acid sequence of SEQ ID NO: 37 and a third polypeptide comprising an amino acid sequence of SEQ ID NO: 29, and the second antigen binding molecule comprises a first polypeptide comprising an amino acid sequence of SEQ ID NO: 28, a second polypeptide comprising an amino acid sequence of SEQ ID NO: 32 and a third polypeptide comprising an amino acid sequence of SEQ ID NO: 35.

27. The pair of antigen binding molecules according to any one of embodiments 1 to 24, wherein the first and the second target-binding domains specifically bind to EGFR, and the first and second cytokine receptor subunits are subunits of the IFNγ receptor complex.

28. The pair of antigen binding molecules according to any one of embodiments 1 to 24 or 27, wherein a) the first antigen binding molecule comprises a first polypeptide comprising an amino acid sequence of SEQ ID NO: 106, a second polypeptide comprising an amino acid sequence of SEQ ID NO: 260 and a third polypeptide comprising an amino acid sequence of SEQ ID NO: 124, and the second antigen binding molecule comprises a first polypeptide comprising an amino acid sequence of SEQ ID NO: 106, a second polypeptide comprising an amino acid sequence of SEQ ID NO: 259 and a third polypeptide comprising an amino acid sequence of SEQ ID NO: 127; or b) the first antigen binding molecule comprises a first polypeptide comprising an amino acid sequence of SEQ ID NO: 106, a second polypeptide comprising an amino acid sequence of SEQ ID NO: 119 and a third polypeptide comprising an amino acid sequence of SEQ ID NO: 256, and the second antigen binding molecule comprises a first polypeptide comprising an amino acid sequence of SEQ ID NO: 106, a second polypeptide comprising an amino acid sequence of SEQ ID NO: 122 and a third polypeptide comprising an amino acid sequence of SEQ ID NO: 255; or c) the first antigen binding molecule comprises a first polypeptide comprising an amino acid sequence of SEQ ID NO: 106, a second polypeptide comprising an amino acid sequence of SEQ ID NO: 261 and a third polypeptide comprising an amino acid sequence of SEQ ID NO: 124, and the second antigen binding molecule comprises a first polypeptide comprising an amino acid sequence of SEQ ID NO: 106, a second polypeptide comprising an amino acid sequence of SEQ ID NO: 258 and a third polypeptide comprising an amino acid sequence of SEQ ID NO: 127; or d) the first antigen binding molecule comprises a first polypeptide comprising an amino acid sequence of SEQ ID NO: 106, a second polypeptide comprising an amino acid sequence of SEQ ID NO: 119 and a third polypeptide comprising an amino acid sequence of SEQ ID NO: 257, and the second antigen binding molecule comprises a first polypeptide comprising an amino acid sequence of SEQ ID NO: 106, a second polypeptide comprising an amino acid sequence of SEQ ID NO: 112 and a third polypeptide comprising an amino acid sequence of SEQ ID NO: 254; or e) the first antigen binding molecule comprises a first polypeptide comprising an amino acid sequence of SEQ ID NO: 106, a second polypeptide comprising an amino acid sequence of SEQ ID NO: 260 and a third polypeptide comprising an amino acid sequence of SEQ ID NO: 124, and the second antigen binding molecule comprises a first polypeptide comprising an amino acid sequence of SEQ ID NO: 106, a second polypeptide comprising an amino acid sequence of SEQ ID NO: 112 and a third polypeptide comprising an amino acid sequence of SEQ ID NO: 255; or f) the first antigen binding molecule comprises a first polypeptide comprising an amino acid sequence of SEQ ID NO: 106, a second polypeptide comprising an amino acid sequence of SEQ ID NO: 119 and a third polypeptide comprising an amino acid sequence of SEQ ID NO: 256, and the second antigen binding molecule comprises a first polypeptide comprising an amino acid sequence of SEQ ID NO: 106, a second polypeptide comprising an amino acid sequence of SEQ ID NO: 259 and a third polypeptide comprising an amino acid sequence of SEQ ID NO: 127; or g) the first antigen binding molecule comprises a first polypeptide comprising an amino acid sequence of SEQ ID NO: 106, a second polypeptide comprising an amino acid sequence of SEQ ID NO: 261 and a third polypeptide comprising an amino acid sequence of SEQ ID NO: 124, and the second antigen binding molecule comprises a first polypeptide comprising an amino acid sequence of SEQ ID NO: 106, a second polypeptide comprising an amino acid sequence of SEQ ID NO: 112 and a third polypeptide comprising an amino acid sequence of SEQ ID NO: 254; or h) the first antigen binding molecule comprises a first polypeptide comprising an amino acid sequence of SEQ ID NO: 106, a second polypeptide comprising an amino acid sequence of SEQ ID NO: 119 and a third polypeptide comprising an amino acid sequence of SEQ ID NO: 257, and the second antigen binding molecule comprises a first polypeptide comprising an amino acid sequence of SEQ ID NO: 106, a second polypeptide comprising an amino acid sequence of SEQ ID NO: 258 and a third polypeptide comprising an amino acid sequence of SEQ ID NO: 127.

29. The pair of antigen binding molecules according to any one of embodiments 1 to 24, wherein the first and the second target-binding domains specifically bind to PD-1, and the first and second cytokine receptor subunits are subunits of the IL-2 receptor complex.

30. The pair of antigen binding molecules according to any one of embodiments 1 to 24 or 29, wherein a) the first antigen binding molecule comprises a first polypeptide comprising an amino acid sequence of SEQ ID NO: 86, a second polypeptide comprising an amino acid sequence of SEQ ID NO: 44 and a third polypeptide comprising an amino acid sequence of SEQ ID NO: 83, and the second antigen binding molecule comprises a first polypeptide comprising an amino acid sequence of SEQ ID NO: 28, a second polypeptide comprising an amino acid sequence of SEQ ID NO: 88 and a third polypeptide comprising an amino acid sequence of SEQ ID NO: 87; or b) the first antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89, and the second antigen binding molecule comprises a first polypeptide comprising an amino acid sequence of SEQ ID NO: 92, a second polypeptide comprising an amino acid sequence of SEQ ID NO: 44 and a third polypeptide comprising an amino acid sequence of SEQ ID NO: 91; or c) the first antigen binding molecule comprises a first polypeptide comprising an amino acid sequence of SEQ ID NO: 28, a second polypeptide comprising an amino acid sequence of SEQ ID NO: 96 and a third polypeptide comprising an amino acid sequence of SEQ ID NO: 95, and the second antigen binding molecule comprises a first polypeptide comprising an amino acid sequence of SEQ ID NO: 28, a second polypeptide comprising an amino acid sequence of SEQ ID NO: 101 and a third polypeptide comprising an amino acid sequence of SEQ ID NO: 89; or d) the first antigen binding molecule comprises a first polypeptide comprising an amino acid sequence of SEQ ID NO: 28, a second polypeptide comprising an amino acid sequence of SEQ ID NO: 99 and a third polypeptide comprising an amino acid sequence of SEQ ID NO: 98, and the second antigen binding molecule comprises a first polypeptide comprising an amino acid sequence of SEQ ID NO: 28, a second polypeptide comprising an amino acid sequence of SEQ ID NO: 88 and a third polypeptide comprising an amino acid sequence of SEQ ID NO: 102; or e) the first antigen binding molecule comprises a first polypeptide comprising an amino acid sequence of SEQ ID NO: 28, a second polypeptide comprising an amino acid sequence of SEQ ID NO: 96 and a third polypeptide comprising an amino acid sequence of SEQ ID NO: 97, and the second antigen binding molecule comprises a first polypeptide comprising an amino acid sequence of SEQ ID NO: 28, a second polypeptide comprising an amino acid sequence of SEQ ID NO: 103 and a third polypeptide comprising an amino acid sequence of SEQ ID NO: 89; or f) the first antigen binding molecule comprises a first polypeptide comprising an amino acid sequence of SEQ ID NO: 28, a second polypeptide comprising an amino acid sequence of SEQ ID NO: 100 and a third polypeptide comprising an amino acid sequence of SEQ ID NO: 98, and the second antigen binding molecule comprises a first polypeptide comprising an amino acid sequence of SEQ ID NO: 28, a second polypeptide comprising an amino acid sequence of SEQ ID NO: 88 and a third polypeptide comprising an amino acid sequence of SEQ ID NO: 104; or g) the first antigen binding molecule comprises a first polypeptide comprising an amino acid sequence of SEQ ID NO: 28, a second polypeptide comprising an amino acid sequence of SEQ ID NO: 98 and a third polypeptide comprising an amino acid sequence of SEQ ID NO: 99, and the second antigen binding molecule comprises a first polypeptide comprising an amino acid sequence of SEQ ID NO: 28, a second polypeptide comprising an amino acid sequence of SEQ ID NO: 101 and a third polypeptide comprising an amino acid sequence of SEQ ID NO: 89; or h) the first antigen binding molecule comprises a first polypeptide comprising an amino acid sequence of SEQ ID NO: 28, a second polypeptide comprising an amino acid sequence of SEQ ID NO: 96 and a third polypeptide comprising an amino acid sequence of SEQ ID NO: 95, and the second antigen binding molecule comprises a first polypeptide comprising an amino acid sequence of SEQ ID NO: 28, a second polypeptide comprising an amino acid sequence of SEQ ID NO: 88 and a third polypeptide comprising an amino acid sequence of SEQ ID NO: 102; or i) the first antigen binding molecule comprises a first polypeptide comprising an amino acid sequence of SEQ ID NO: 28, a second polypeptide comprising an amino acid sequence of SEQ ID NO: 100 and a third polypeptide comprising an amino acid sequence of SEQ ID NO: 98, and the second antigen binding molecule comprises a first polypeptide comprising an amino acid sequence of SEQ ID NO: 28, a second polypeptide comprising an amino acid sequence of SEQ ID NO: 103 and a third polypeptide comprising an amino acid sequence of SEQ ID NO: 89; or j) the first antigen binding molecule comprises a first polypeptide comprising an amino acid sequence of SEQ ID NO: 28, a second polypeptide comprising an amino acid sequence of SEQ ID NO: 96 and a third polypeptide comprising an amino acid sequence of SEQ ID NO: 97, and the second antigen binding molecule comprises a first polypeptide comprising an amino acid sequence of SEQ ID NO: 28, a second polypeptide comprising an amino acid sequence of SEQ ID NO: 88 and a third polypeptide comprising an amino acid sequence of SEQ ID NO: 104.

31. The pair of antigen binding molecules according to any one of embodiments 1 to 24, wherein the first and the second target-binding domains specifically bind to LAG-3, and the first and second cytokine receptor subunits are subunits of the IL-2 receptor complex.

32. The pair of antigen binding molecules according to any one of embodiments 1 to 24 or 31, wherein a) first antigen binding molecule comprises a first polypeptide comprising an amino acid sequence of SEQ ID NO: 106, a second polypeptide comprising an amino acid sequence of SEQ ID NO: 155 and a third polypeptide comprising an amino acid sequence of SEQ ID NO: 154, and the second antigen binding molecule comprises a first polypeptide comprising an amino acid sequence of SEQ ID NO: 106, a second polypeptide comprising an amino acid sequence of SEQ ID NO: 165 and a third polypeptide comprising an amino acid sequence of SEQ ID NO: 163.; or b) the first antigen binding molecule comprises a first polypeptide comprising an amino acid sequence of SEQ ID NO: 106, a second polypeptide comprising an amino acid sequence of SEQ ID NO: 161 and a third polypeptide comprising an amino acid sequence of SEQ ID NO: 160, and the second antigen binding molecule comprises a first polypeptide comprising an amino acid sequence of SEQ ID NO: 106, a second polypeptide comprising an amino acid sequence of SEQ ID NO: 158 and a third polypeptide comprising an amino acid sequence of SEQ ID NO: 159; or c) the first antigen binding molecule comprises a first polypeptide comprising an amino acid sequence of SEQ ID NO: 106, a second polypeptide comprising an amino acid sequence of SEQ ID NO: 155 and a third polypeptide comprising an amino acid sequence of SEQ ID NO: 156, and the second antigen binding molecule comprises a first polypeptide comprising an amino acid sequence of SEQ ID NO: 106, a second polypeptide comprising an amino acid sequence of SEQ ID NO: 164 and a third polypeptide comprising an amino acid sequence of SEQ ID NO: 163; or d) the first antigen binding molecule comprises a first polypeptide comprising an amino acid sequence of SEQ ID NO: 106, a second polypeptide comprising an amino acid sequence of SEQ ID NO: 162 and a third polypeptide comprising an amino acid sequence of SEQ ID NO: 160, and the second antigen binding molecule comprises a first polypeptide comprising an amino acid sequence of SEQ ID NO: 106, a second polypeptide comprising an amino acid sequence of SEQ ID NO: 158 and a third polypeptide comprising an amino acid sequence of SEQ ID NO: 157; or e) the first antigen binding molecule comprises a first polypeptide comprising an amino acid sequence of SEQ ID NO: 106, a second polypeptide comprising an amino acid sequence of SEQ ID NO: 161 and a third polypeptide comprising an amino acid sequence of SEQ ID NO: 160, and the second antigen binding molecule comprises a first polypeptide comprising an amino acid sequence of SEQ ID NO: 106, a second polypeptide comprising an amino acid sequence of SEQ ID NO: 165 and a third polypeptide comprising an amino acid sequence of SEQ ID NO: 163; or f) the first antigen binding molecule comprises a first polypeptide comprising an amino acid sequence of SEQ ID NO: 106, a second polypeptide comprising an amino acid sequence of SEQ ID NO: 155 and a third polypeptide comprising an amino acid sequence of SEQ ID NO: 154, and the second antigen binding molecule comprises a first polypeptide comprising an amino acid sequence of SEQ ID NO: 106, a second polypeptide comprising an amino acid sequence of SEQ ID NO: 158 and a third polypeptide comprising an amino acid sequence of SEQ ID NO: 159; or g) the first antigen binding molecule comprises a first polypeptide comprising an amino acid sequence of SEQ ID NO: 106, a second polypeptide comprising an amino acid sequence of SEQ ID NO: 162 and a third polypeptide comprising an amino acid sequence of SEQ ID NO: 160, and the second antigen binding molecule comprises a first polypeptide comprising an amino acid sequence of SEQ ID NO: 106, a second polypeptide comprising an amino acid sequence of SEQ ID NO: 164 and a third polypeptide comprising an amino acid sequence of SEQ ID NO: 163; or h) the first antigen binding molecule comprises a first polypeptide comprising an amino acid sequence of SEQ ID NO: 106, a second polypeptide comprising an amino acid sequence of SEQ ID NO: 155 and a third polypeptide comprising an amino acid sequence of SEQ ID NO: 156, and the second antigen binding molecule comprises a first polypeptide comprising an amino acid sequence of SEQ ID NO: 106, a second polypeptide comprising an amino acid sequence of SEQ ID NO: 158 and a third polypeptide comprising an amino acid sequence of SEQ ID NO: 157.

33. The pair of antigen binding molecules according to any one of embodiments 1 to 24, wherein the first and the second target-binding domains specifically bind to EGFR, and the first and second cytokine receptor subunits are subunits of the IL-2 receptor complex.

34. The pair of antigen binding molecules according to any one of embodiments 1 to 24 or 33, wherein a) first antigen binding molecule comprises a first polypeptide comprising an amino acid sequence of SEQ ID NO: 106, a second polypeptide comprising an amino acid sequence of SEQ ID NO: 155 and a third polypeptide comprising an amino acid sequence of SEQ ID NO: 154, and the second antigen binding molecule comprises a first polypeptide comprising an amino acid sequence of SEQ ID NO: 106, a second polypeptide comprising an amino acid sequence of SEQ ID NO: 165 and a third polypeptide comprising an amino acid sequence of SEQ ID NO: 163; or b) the first antigen binding molecule comprises a first polypeptide comprising an amino acid sequence of SEQ ID NO: 106, a second polypeptide comprising an amino acid sequence of SEQ ID NO: 161 and a third polypeptide comprising an amino acid sequence of SEQ ID NO: 160, and the second antigen binding molecule comprises a first polypeptide comprising an amino acid sequence of SEQ ID NO: 106, a second polypeptide comprising an amino acid sequence of SEQ ID NO: 158 and a third polypeptide comprising an amino acid sequence of SEQ ID NO: 159.; or c) the first antigen binding molecule comprises a first polypeptide comprising an amino acid sequence of SEQ ID NO: 106, a second polypeptide comprising an amino acid sequence of SEQ ID NO: 155 and a third polypeptide comprising an amino acid sequence of SEQ ID NO: 156, and the second antigen binding molecule comprises a first polypeptide comprising an amino acid sequence of SEQ ID NO: 106, a second polypeptide comprising an amino acid sequence of SEQ ID NO: 164 and a third polypeptide comprising an amino acid sequence of SEQ ID NO: 163; or d) the first antigen binding molecule comprises a first polypeptide comprising an amino acid sequence of SEQ ID NO: 106, a second polypeptide comprising an amino acid sequence of SEQ ID NO: 162 and a third polypeptide comprising an amino acid sequence of SEQ ID NO: 160, and the second antigen binding molecule comprises a first polypeptide comprising an amino acid sequence of SEQ ID NO: 106, a second polypeptide comprising an amino acid sequence of SEQ ID NO: 158 and a third polypeptide comprising an amino acid sequence of SEQ ID NO: 157; or e) the first antigen binding molecule comprises a first polypeptide comprising an amino acid sequence of SEQ ID NO: 106, a second polypeptide comprising an amino acid sequence of SEQ ID NO: 161 and a third polypeptide comprising an amino acid sequence of SEQ ID NO: 160, and the second antigen binding molecule comprises a first polypeptide comprising an amino acid sequence of SEQ ID NO: 106, a second polypeptide comprising an amino acid sequence of SEQ ID NO: 165 and a third polypeptide comprising an amino acid sequence of SEQ ID NO: 163; or f) the first antigen binding molecule comprises a first polypeptide comprising an amino acid sequence of SEQ ID NO: 106, a second polypeptide comprising an amino acid sequence of SEQ ID NO: 155 and a third polypeptide comprising an amino acid sequence of SEQ ID NO: 154, and the second antigen binding molecule comprises a first polypeptide comprising an amino acid sequence of SEQ ID NO: 106, a second polypeptide comprising an amino acid sequence of SEQ ID NO: 158 and a third polypeptide comprising an amino acid sequence of SEQ ID NO: 159; or g) the first antigen binding molecule comprises a first polypeptide comprising an amino acid sequence of SEQ ID NO: 106, a second polypeptide comprising an amino acid sequence of SEQ ID NO: 162 and a third polypeptide comprising an amino acid sequence of SEQ ID NO: 160, and the second antigen binding molecule comprises a first polypeptide comprising an amino acid sequence of SEQ ID NO: 106, a second polypeptide comprising an amino acid sequence of SEQ ID NO: 164 and a third polypeptide comprising an amino acid sequence of SEQ ID NO: 163; or h) the first antigen binding molecule comprises a first polypeptide comprising an amino acid sequence of SEQ ID NO: 106, a second polypeptide comprising an amino acid sequence of SEQ ID NO: 155 and a third polypeptide comprising an amino acid sequence of SEQ ID NO: 156, and the second antigen binding molecule comprises a first polypeptide comprising an amino acid sequence of SEQ ID NO: 106, a second polypeptide comprising an amino acid sequence of SEQ ID NO: 158 and a third polypeptide comprising an amino acid sequence of SEQ ID NO: 157.

35. The pair of antigen binding molecules according to any one of embodiments 1 to 24, wherein the first and the second target-binding domains specifically bind to FAP, and the first and second cytokine receptor subunits are subunits of the IL-2 receptor complex.

36. The pair of antigen binding molecules according to any one of embodiments 1 to 24 or 35, wherein a) the first antigen binding molecule comprises a first polypeptide comprising an amino acid sequence of SEQ ID NO: 106, a second polypeptide comprising an amino acid sequence of SEQ ID NO: 110 and a third polypeptide comprising an amino acid sequence of SEQ ID NO: 25, and the second antigen binding molecule comprises a first polypeptide comprising an amino acid sequence of SEQ ID NO: 106, a second polypeptide comprising an amino acid sequence of SEQ ID NO: 113 and a third polypeptide comprising an amino acid sequence of SEQ ID NO: 29; or b) the first antigen binding molecule comprises a first polypeptide comprising an amino acid sequence of SEQ ID NO: 106, a second polypeptide comprising an amino acid sequence of SEQ ID NO: 32 and a third polypeptide comprising an amino acid sequence of SEQ ID NO: 105, and the second antigen binding molecule comprises a first polypeptide comprising an amino acid sequence of SEQ ID NO: 106, a second polypeptide comprising an amino acid sequence of SEQ ID NO: 34 and a third polypeptide comprising an amino acid sequence of SEQ ID NO: 109; or c) the first antigen binding molecule comprises a first polypeptide comprising an amino acid sequence of SEQ ID NO: 106, a second polypeptide comprising an amino acid sequence of SEQ ID NO: 111 and a third polypeptide comprising an amino acid sequence of SEQ ID NO: 25, and the second antigen binding molecule comprises a first polypeptide comprising an amino acid sequence of SEQ ID NO: 106, a second polypeptide comprising an amino acid sequence of SEQ ID NO: 112 and a third polypeptide comprising an amino acid sequence of SEQ ID NO: 29; or d) the first antigen binding molecule comprises a first polypeptide comprising an amino acid sequence of SEQ ID NO: 106, a second polypeptide comprising an amino acid sequence of SEQ ID NO: 32 and a third polypeptide comprising an amino acid sequence of SEQ ID NO: 107, and the second antigen binding molecule comprises a first polypeptide comprising an amino acid sequence of SEQ ID NO: 106, a second polypeptide comprising an amino acid sequence of SEQ ID NO: 34 and a third polypeptide comprising an amino acid sequence of SEQ ID NO: 108; or e) the first antigen binding molecule comprises a first polypeptide comprising an amino acid sequence of SEQ ID NO: 106, a second polypeptide comprising an amino acid sequence of SEQ ID NO: 110 and a third polypeptide comprising an amino acid sequence of SEQ ID NO: 25, and the second antigen binding molecule comprises a first polypeptide comprising an amino acid sequence of SEQ ID NO: 106, a second polypeptide comprising an amino acid sequence of SEQ ID NO: 34 and a third polypeptide comprising an amino acid sequence of SEQ ID NO: 109; or f) the first antigen binding molecule comprises a first polypeptide comprising an amino acid sequence of SEQ ID NO: 106, a second polypeptide comprising an amino acid sequence of SEQ ID NO: 32 and a third polypeptide comprising an amino acid sequence of SEQ ID NO: 105, and the second antigen binding molecule comprises a first polypeptide comprising an amino acid sequence of SEQ ID NO: 106, a second polypeptide comprising an amino acid sequence of SEQ ID NO: 113 and a third polypeptide comprising an amino acid sequence of SEQ ID NO: 29; or g) the first antigen binding molecule comprises a first polypeptide comprising an amino acid sequence of SEQ ID NO: 106, a second polypeptide comprising an amino acid sequence of SEQ ID NO: 111 and a third polypeptide comprising an amino acid sequence of SEQ ID NO: 25, and the second antigen binding molecule comprises a first polypeptide comprising an amino acid sequence of SEQ ID NO: 106, a second polypeptide comprising an amino acid sequence of SEQ ID NO: 34 and a third polypeptide comprising an amino acid sequence of SEQ ID NO: 108; or h) the first antigen binding molecule comprises a first polypeptide comprising an amino acid sequence of SEQ ID NO: 106, a second polypeptide comprising an amino acid sequence of SEQ ID NO: 32 and a third polypeptide comprising an amino acid sequence of SEQ ID NO: 107, and the second antigen binding molecule comprises a first polypeptide comprising an amino acid sequence of SEQ ID NO: 106, a second polypeptide comprising an amino acid sequence of SEQ ID NO: 112 and a third polypeptide comprising an amino acid sequence of SEQ ID NO: 29.

37. The pair of antigen binding molecules according to any one of embodiments 1 to 24, wherein the first and the second target-binding domains specifically bind to Her2, and the first and second cytokine receptor subunits are subunits of the IL-2 receptor complex.

38. The pair of antigen binding molecules according to any one of embodiments 1 to 24 or 37, wherein a) first antigen binding molecule comprises a first polypeptide comprising an amino acid sequence of SEQ ID NO: 106, a second polypeptide comprising an amino acid sequence of SEQ ID NO: 136 and a third polypeptide comprising an amino acid sequence of SEQ ID NO: 134, and the second antigen binding molecule comprises a first polypeptide comprising an amino acid sequence of SEQ ID NO: 106, a second polypeptide comprising an amino acid sequence of SEQ ID NO: 138 and a third polypeptide comprising an amino acid sequence of SEQ ID NO: 137; or b) the first antigen binding molecule comprises a first polypeptide comprising an amino acid sequence of SEQ ID NO: 106, a second polypeptide comprising an amino acid sequence of SEQ ID NO: 135 and a third polypeptide comprising an amino acid sequence of SEQ ID NO: 134, and the second antigen binding molecule comprises a first polypeptide comprising an amino acid sequence of SEQ ID NO: 106, a second polypeptide comprising an amino acid sequence of SEQ ID NO: 138 and a third polypeptide comprising an amino acid sequence of SEQ ID NO: 139.

39. The pair of antigen binding molecules according to any one of embodiments 1 to 24, wherein the first and the second target-binding domains specifically bind to Her3, and the first and second cytokine receptor subunits are subunits of the IL-2 receptor complex.

40. The pair of antigen binding molecules according to any one of embodiments 1 to 24 or 39, wherein a) the first antigen binding molecule comprises a first polypeptide comprising an amino acid sequence of SEQ ID NO: 106, a second polypeptide comprising an amino acid sequence of SEQ ID NO: 146 and a third polypeptide comprising an amino acid sequence of SEQ ID NO: 144, and the second antigen binding molecule comprises a first polypeptide comprising an amino acid sequence of SEQ ID NO: 106, a second polypeptide comprising an amino acid sequence of SEQ ID NO: 148 and a third polypeptide comprising an amino acid sequence of SEQ ID NO: 147; or b) the first antigen binding molecule comprises a first polypeptide comprising an amino acid sequence of SEQ ID NO: 106, a second polypeptide comprising an amino acid sequence of SEQ ID NO: 145 and a third polypeptide comprising an amino acid sequence of SEQ ID NO: 144, and the second antigen binding molecule comprises a first polypeptide comprising an amino acid sequence of SEQ ID NO: 106, a second polypeptide comprising an amino acid sequence of SEQ ID NO: 148 and a third polypeptide comprising an amino acid sequence of SEQ ID NO: 149.

41. The pair of antigen binding molecules according to any one of embodiments 1 to 24, wherein the first and the second target-binding domains specifically bind to PD-1, and the first and second cytokine receptor subunits are subunits of the IL-7 receptor complex.

42. The pair of antigen binding molecules according to any one of embodiments 1 to 24 and 41, wherein a) the first antigen binding molecule comprises a first polypeptide comprising an amino acid sequence of SEQ ID NO: 106, a second polypeptide comprising an amino acid sequence of SEQ ID NO: 194 and a third polypeptide comprising an amino acid sequence of SEQ ID NO: 193, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89; or b) the first antigen binding molecule comprises a first polypeptide comprising an amino acid sequence of SEQ ID NO: 106, a second polypeptide comprising an amino acid sequence of SEQ ID NO: 195 and a third polypeptide comprising an amino acid sequence of SEQ ID NO: 124, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89; or c) the first antigen binding molecule comprises a first polypeptide comprising an amino acid sequence of SEQ ID NO: 106, a second polypeptide comprising an amino acid sequence of SEQ ID NO: 196 and a third polypeptide comprising an amino acid sequence of SEQ ID NO: 124, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89; or d) the first antigen binding molecule comprises a first polypeptide comprising an amino acid sequence of SEQ ID NO: 106, a second polypeptide comprising an amino acid sequence of SEQ ID NO: 197 and a third polypeptide comprising an amino acid sequence of SEQ ID NO: 124, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89; or d) the first antigen binding molecule comprises a first polypeptide comprising an amino acid sequence of SEQ ID NO: 106, a second polypeptide comprising an amino acid sequence of SEQ ID NO: 198 and a third polypeptide comprising an amino acid sequence of SEQ ID NO: 124, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89; or e) the first antigen binding molecule comprises a first polypeptide comprising an amino acid sequence of SEQ ID NO: 106, a second polypeptide comprising an amino acid sequence of SEQ ID NO: 199 and a third polypeptide comprising an amino acid sequence of SEQ ID NO: 124, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89; or f) the first antigen binding molecule comprises a first polypeptide comprising an amino acid sequence of SEQ ID NO: 106, a second polypeptide comprising an amino acid sequence of SEQ ID NO: 200 and a third polypeptide comprising an amino acid sequence of SEQ ID NO: 124, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89; or g) the first antigen binding molecule comprises a first polypeptide comprising an amino acid sequence of SEQ ID NO: 106, a second polypeptide comprising an amino acid sequence of SEQ ID NO: 201 and a third polypeptide comprising an amino acid sequence of SEQ ID NO: 124, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89; or h) the first antigen binding molecule comprises a first polypeptide comprising an amino acid sequence of SEQ ID NO: 106, a second polypeptide comprising an amino acid sequence of SEQ ID NO: 202 and a third polypeptide comprising an amino acid sequence of SEQ ID NO: 124, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89; or i) the first antigen binding molecule comprises a first polypeptide comprising an amino acid sequence of SEQ ID NO: 106, a second polypeptide comprising an amino acid sequence of SEQ ID NO: 203 and a third polypeptide comprising an amino acid sequence of SEQ ID NO: 124, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89; or j) the first antigen binding molecule comprises a first polypeptide comprising an amino acid sequence of SEQ ID NO: 106, a second polypeptide comprising an amino acid sequence of SEQ ID NO: 204 and a third polypeptide comprising an amino acid sequence of SEQ ID NO: 124, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89; or k) the first antigen binding molecule comprises a first polypeptide comprising an amino acid sequence of SEQ ID NO: 106, a second polypeptide comprising an amino acid sequence of SEQ ID NO: 205 and a third polypeptide comprising an amino acid sequence of SEQ ID NO: 124, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89.

43. The pair of antigen binding molecules according to any one of embodiments 1 to 21, wherein the first target-binding domain is fused at its C-terminus of CH1₁ to the N-terminus of the first Fc domain subunit and the first cytokine receptor-binding domain is fused at its C-terminus to the N-terminus of the second Fc domain subunit and the second target-binding domain is fused at its C-terminus of CH1₂ to the N-terminus of the first Fc domain subunit and the second cytokine receptor-binding domain is fused at its C-terminus to the N-terminus of the second Fc domain subunit.

44. The pair of antigen binding molecules according to any one of embodiments 1 to 21 or 43, wherein a) the first antigen binding molecule comprises a first polypeptide comprising in order from the N-terminus to C-terminus VH₁, CH1₁ and a first Fc domain subunit, a second polypeptide comprising in order from the N-terminus to C-terminus VL₁ and CL₁, and a third polypeptide comprising in order from the N-terminus to C-terminus the first cytokine receptor-binding domain and a second Fc domain subunit, and the second antigen binding molecule comprises a first polypeptide comprising in order from the N-terminus to C-terminus VH₂, CH1₂ and a first Fc domain subunit, a second polypeptide comprising in order from the N-terminus to C-terminus VL₂ and CL₂, and a third polypeptide comprising in order from the N-terminus to C-terminus the second cytokine receptor-binding domain and a second Fc domain subunit; or b) the first antigen binding molecule comprises a first polypeptide comprising in order from the N-terminus to C-terminus VL₁, CH1₁ and a first Fc domain subunit, a second polypeptide comprising in order from the N-terminus to C-terminus VH₁ and CL₁, and a third polypeptide comprising in order from the N-terminus to C-terminus the first cytokine receptor-binding domain and a second Fc domain subunit, and the second antigen binding molecule comprises a first polypeptide comprising in order from the N-terminus to C-terminus VH₂, CH1₂ and a first Fc domain subunit, a second polypeptide comprising in order from the N-terminus to C-terminus VL₂ and CL₂, and a third polypeptide comprising in order from the N-terminus to C-terminus the second cytokine receptor-binding domain and a second Fc domain subunit; wherein VH₁, VL₁, CH1₁ and CL₁ form the first target-binding domain and VH₂, VL₂, CH1₂, and CL₂, form the second target-binding domain.

45. The pair of antigen binding molecules according to any one of embodiments 1 to 21 or 43 to 44, wherein a) the first antigen binding molecule comprises a first polypeptide comprising an amino acid sequence of SEQ ID NO: 32, a second polypeptide comprising an amino acid sequence of SEQ ID NO: 25 and a third polypeptide comprising an amino acid sequence of SEQ ID NO: 39, and the second antigen binding molecule comprises a first polypeptide comprising an amino acid sequence of SEQ ID NO: 34, a second polypeptide comprising an amino acid sequence of SEQ ID NO: 29 and a third polypeptide comprising an amino acid sequence of SEQ ID NO: 40; or b) the first antigen binding molecule comprises a first polypeptide comprising an amino acid sequence of SEQ ID NO: 34, a second polypeptide comprising an amino acid sequence of SEQ ID NO: 29 and a third polypeptide comprising an amino acid sequence of SEQ ID NO: 39, and the second antigen binding molecule comprises a first polypeptide comprising an amino acid sequence of SEQ ID NO: 32, a second polypeptide comprising an amino acid sequence of SEQ ID NO: 25 and a third polypeptide comprising an amino acid sequence of SEQ ID NO: 40; or c) the first antigen binding molecule comprises a first polypeptide comprising an amino acid sequence of SEQ ID NO: 237, a second polypeptide comprising an amino acid sequence of SEQ ID NO: 32 and a third polypeptide comprising an amino acid sequence of SEQ ID NO: 25, and the second antigen binding molecule comprises a first polypeptide comprising an amino acid sequence of SEQ ID NO: 238, a second polypeptide comprising an amino acid sequence of SEQ ID NO: 34 and a third polypeptide comprising an amino acid sequence of SEQ ID NO: 29; or d) the first antigen binding molecule comprises a first polypeptide comprising an amino acid sequence of SEQ ID NO: 235, a second polypeptide comprising an amino acid sequence of SEQ ID NO: 32 and a third polypeptide comprising an amino acid sequence of SEQ ID NO: 25, and the second antigen binding molecule comprises a first polypeptide comprising an amino acid sequence of SEQ ID NO: 236, a second polypeptide comprising an amino acid sequence of SEQ ID NO: 34 and a third polypeptide comprising an amino acid sequence of SEQ ID NO: 29; or e) the first antigen binding molecule comprises a first polypeptide comprising an amino acid sequence of SEQ ID NO: 239, a second polypeptide comprising an amino acid sequence of SEQ ID NO: 32 and a third polypeptide comprising an amino acid sequence of SEQ ID NO: 25, and the second antigen binding molecule comprises a first polypeptide comprising an amino acid sequence of SEQ ID NO: 240, a second polypeptide comprising an amino acid sequence of SEQ ID NO: 34 and a third polypeptide comprising an amino acid sequence of SEQ ID NO: 29; or f) the first antigen binding molecule comprises a first polypeptide comprising an amino acid sequence of SEQ ID NO: 235, a second polypeptide comprising an amino acid sequence of SEQ ID NO: 32 and a third polypeptide comprising an amino acid sequence of SEQ ID NO: 25, and the second antigen binding molecule comprises a first polypeptide comprising an amino acid sequence of SEQ ID NO: 237, a second polypeptide comprising an amino acid sequence of SEQ ID NO: 34 and a third polypeptide comprising an amino acid sequence of SEQ ID NO: 29; or g) the first antigen binding molecule comprises a first polypeptide comprising an amino acid sequence of SEQ ID NO: 236, a second polypeptide comprising an amino acid sequence of SEQ ID NO: 32 and a third polypeptide comprising an amino acid sequence of SEQ ID NO: 25, and the second antigen binding molecule comprises a first polypeptide comprising an amino acid sequence of SEQ ID NO: 235, a second polypeptide comprising an amino acid sequence of SEQ ID NO: 34 and a third polypeptide comprising an amino acid sequence of SEQ ID NO: 29; or h) the first antigen binding molecule comprises a first polypeptide comprising an amino acid sequence of SEQ ID NO: 240, a second polypeptide comprising an amino acid sequence of SEQ ID NO: 32 and a third polypeptide comprising an amino acid sequence of SEQ ID NO: 25, and the second antigen binding molecule comprises a first polypeptide comprising an amino acid sequence of SEQ ID NO: 239, a second polypeptide comprising an amino acid sequence of SEQ ID NO: 34 and a third polypeptide comprising an amino acid sequence of SEQ ID NO: 29.

46. The pair of antigen binding molecules according to any one of embodiments 1 to 21, wherein the first target-binding domain is fused at its C-terminus of CH1₁ to the N-terminus of one of the Fc domain subunits and the first cytokine receptor-binding domain is fused at its N-terminus to the C-terminus of the same Fc domain subunit and the second target-binding domain is fused at its C-terminus of CH1₂ to the N-terminus of one of the Fc domain subunits and the second cytokine receptor-binding domain is fused at its N-terminus to the C-terminus of the same Fc domain subunit.

47. The pair of antigen binding molecules according to any one of embodiments 1 to 21 or 46, wherein a) the first antigen binding molecule comprises a first polypeptide comprising a first Fc domain subunit, a second polypeptide comprising in order from the N-terminus to C-terminus a VH₁, a CH1₁, a second Fc domain subunit and a the first cytokine receptor-binding domain, and a third polypeptide comprising in order from the N-terminus to C-terminus a VL₁ and a CL₁; and the second antigen binding molecule comprises a first polypeptide comprising a first Fc domain subunit, a second polypeptide comprising in order from the N-terminus to C-terminus a VH₂, a CH1₂, a second Fc domain subunit and a the second cytokine receptor-binding domain, and a third polypeptide comprising in order from the N-terminus to C-terminus VL₂ and CL₂; or b) the first antigen binding molecule comprises a first polypeptide comprising a first Fc domain subunit, a second polypeptide comprising in order from the N-terminus to C-terminus a VL₁, a CH1₁, a second Fc domain subunit and a the first cytokine receptor-binding domain, and a third polypeptide comprising in order from the N-terminus to C-terminus a VH₁ and a CL₁; and the second antigen binding molecule comprises a first polypeptide comprising a first Fc domain subunit, a second polypeptide comprising in order from the N-terminus to C-terminus a VH₂, a CH1₂, a second Fc domain subunit and the second cytokine receptor-binding domain, and a third polypeptide comprising in order from the N-terminus to C-terminus a VL₂ and a CL₂; or c) the first antigen binding molecule comprises a first polypeptide comprising a first Fc domain subunit, a second polypeptide comprising in order from the N-terminus to C-terminus a VL₁, a CH1₁, a second Fc domain subunit and a the first cytokine receptor-binding domain, and a third polypeptide comprising in order from the N-terminus to C-terminus a VH₁ and a CL₁; and the second antigen binding molecule comprises a first polypeptide comprising a first Fc domain subunit, a second polypeptide comprising in order from the N-terminus to C-terminus a VH₂, a CH1₂, a second Fc domain subunit and a the second cytokine receptor-binding domain, and a third polypeptide comprising in order from the N-terminus to C-terminus VL₂ and CL₂; wherein VH₁, VL₁, CH1₁ and CL₁ form the first target-binding domain and VH₂, VL₂, CH1₂, and CL₂, form the second target-binding domain.

48. The pair of antigen binding molecules according to any one of embodiments 1 to 21 or 46 to 47, wherein a) the first antigen binding molecule comprises a first polypeptide comprising an amino acid sequence of SEQ ID NO: 28, a second polypeptide comprising an amino acid sequence of SEQ ID NO: 42 and a third polypeptide comprising an amino acid sequence of SEQ ID NO: 41, and the second antigen binding molecule comprises a first polypeptide comprising an amino acid sequence of SEQ ID NO: 45, a second polypeptide comprising an amino acid sequence of SEQ ID NO: 44 and a third polypeptide comprising an amino acid sequence of SEQ ID NO: 43; or b) the first antigen binding molecule comprises a first polypeptide comprising an amino acid sequence of SEQ ID NO: 28, a second polypeptide comprising an amino acid sequence of SEQ ID NO: 46 and a third polypeptide comprising an amino acid sequence of SEQ ID NO: 41, and the second antigen binding molecule comprises a first polypeptide comprising an amino acid sequence of SEQ ID NO: 47, a second polypeptide comprising an amino acid sequence of SEQ ID NO: 44 and a third polypeptide comprising an amino acid sequence of SEQ ID NO: 43; or c) the first antigen binding molecule comprises a first polypeptide comprising an amino acid sequence of SEQ ID NO: 241, a second polypeptide comprising an amino acid sequence of SEQ ID NO: 246 and a third polypeptide comprising an amino acid sequence of SEQ ID NO: 25, and the second antigen binding molecule comprises a first polypeptide comprising an amino acid sequence of SEQ ID NO: 241, a second polypeptide comprising an amino acid sequence of SEQ ID NO: 249 and a third polypeptide comprising an amino acid sequence of SEQ ID NO: 29; or d) the first antigen binding molecule comprises a first polypeptide comprising an amino acid sequence of SEQ ID NO: 241, a second polypeptide comprising an amino acid sequence of SEQ ID NO: 242 and a third polypeptide comprising an amino acid sequence of SEQ ID NO: 25, and the second antigen binding molecule comprises a first polypeptide comprising an amino acid sequence of SEQ ID NO: 241, a second polypeptide comprising an amino acid sequence of SEQ ID NO: 245 and a third polypeptide comprising an amino acid sequence of SEQ ID NO: 29; or e) the first antigen binding molecule comprising a first polypeptide comprises an amino acid sequence of SEQ ID NO: 241, a second polypeptide comprising an amino acid sequence of SEQ ID NO: 250 and a third polypeptide comprising an amino acid sequence of SEQ ID NO: 25, and the second antigen binding molecule comprises a first polypeptide comprising an amino acid sequence of SEQ ID NO: 241, a second polypeptide comprising an amino acid sequence of SEQ ID NO: 253 and a third polypeptide comprising an amino acid sequence of SEQ ID NO: 29; or f) the first antigen binding molecule comprises a first polypeptide comprising an amino acid sequence of SEQ ID NO: 241, a second polypeptide comprising an amino acid sequence of SEQ ID NO: 248 and a third polypeptide comprising an amino acid sequence of SEQ ID NO: 25, and the second antigen binding molecule comprises a first polypeptide comprising an amino acid sequence of SEQ ID NO: 241, a second polypeptide comprising an amino acid sequence of SEQ ID NO: 247 and a third polypeptide comprising an amino acid sequence of SEQ ID NO: 29; or g) the first antigen binding molecule comprises a first polypeptide comprising an amino acid sequence of SEQ ID NO: 241, a second polypeptide comprising an amino acid sequence of SEQ ID NO: 244 and a third polypeptide comprising an amino acid sequence of SEQ ID NO: 25, and the second antigen binding molecule comprises a first polypeptide comprising an amino acid sequence of SEQ ID NO: 241, a second polypeptide comprising an amino acid sequence of SEQ ID NO: 243 and a third polypeptide comprising an amino acid sequence of SEQ ID NO: 29; or h) the first antigen binding molecule comprises a first polypeptide comprising an amino acid sequence of SEQ ID NO: 241, a second polypeptide comprising an amino acid sequence of SEQ ID NO: 252 and a third polypeptide comprising an amino acid sequence of SEQ ID NO: 25, and the second antigen binding molecule comprises a first polypeptide comprising an amino acid sequence of SEQ ID NO: 241, a second polypeptide comprising an amino acid sequence of SEQ ID NO: 251 and a third polypeptide comprising an amino acid sequence of SEQ ID NO: 29; or i) the first antigen binding molecule comprises a first polypeptide comprising an amino acid sequence of SEQ ID NO: 28, a second polypeptide comprising an amino acid sequence of SEQ ID NO: 42 and a third polypeptide comprising an amino acid sequence of SEQ ID NO: 42, and the second antigen binding molecule comprises a first polypeptide comprising an amino acid sequence of SEQ ID NO: 241, a second polypeptide comprising an amino acid sequence of SEQ ID NO: 245 and a third polypeptide comprising an amino acid sequence of SEQ ID NO: 29.

49. The pair of antigen binding molecules according to any one of embodiments 1 to 11, wherein the first and second cytokine receptor-binding domains are Fab molecules.

50. The pair of antigen binding molecules according to embodiments 49, wherein a) the first cytokine receptor-binding domain comprises a VH domain comprising the amino acid sequence of SEQ ID NO: 282 and a VL domain comprising the amino acid sequence of SEQ ID NO: 283 or VH domain comprising the amino acid sequence of SEQ ID NO: 284 and a VL domain comprising the amino acid sequence of SEQ ID NO: 285, and the second cytokine receptor domain comprises a VH domain comprising the amino acid sequence of SEQ ID NO: 286 and a VL domain comprising the amino acid sequence of SEQ ID NO: 287 or VH domain comprising the amino acid sequence of SEQ ID NO: 288 and a VL domain comprising the amino acid sequence of SEQ ID NO: 289; or b) the first cytokine receptor-binding domain comprises a VH domain comprising the amino acid sequence of SEQ ID NO: 286 and a VL domain comprising the amino acid sequence of SEQ ID NO: 287 or VH domain comprising the amino acid sequence of SEQ ID NO: 288 and a VL domain comprising the amino acid sequence of SEQ ID NO: 289, and the second cytokine receptor domain comprises a VH domain comprising the amino acid sequence of SEQ ID NO: 282 and a VL domain comprising the amino acid sequence of SEQ ID NO: 283 or VH domain comprising the amino acid sequence of SEQ ID NO: 284 and a VL domain comprising the amino acid sequence of SEQ ID NO: 285.

51. The pair of antigen binding molecules according to any one of embodiments 1 to 11, wherein the first and second cytokine receptor-binding domains are scFv molecules.

52. The pair of antigen binding molecules according to embodiments 51, wherein a) the first cytokine receptor-binding domain comprises a VH domain comprising the amino acid sequence of SEQ ID NO: 290 and a VL domain comprising the amino acid sequence of SEQ ID NO: 291, and the second cytokine receptor domain comprises a VH domain comprising the amino acid sequence of SEQ ID NO: 292 and a VL domain comprising the amino acid sequence of SEQ ID NO: 293; or b) the first cytokine receptor-binding domain comprises a VH domain comprising the amino acid sequence of SEQ ID NO: 292 and a VL domain comprising the amino acid sequence of SEQ ID NO: 293, and the second cytokine receptor domain comprises a VH domain comprising the amino acid sequence of SEQ ID NO: 290 and a VL domain comprising the amino acid sequence of SEQ ID NO: 291.

53. The pair of antigen binding molecules according to any one of embodiments 49 to 52, wherein the Fc domains of the first and second antigen binding molecules comprise a first Fc domain subunit and a second Fc domain subunit.

54. The pair of antigen binding molecules according to any one of embodiments 49 to 53, wherein the Fc domains of the first and second antigen binding molecules are IgG, particularly an IgG₁, Fc domains.

55. The pair of antigen binding molecules according to any one of embodiments 49 to 54, wherein the Fc domains of the first and second antigen binding molecules are human Fc domains.

56. The pair of antigen binding molecules according to any one of embodiments 49 to 55, wherein the Fc domains of the first and second antigen binding molecules comprise a modification promoting the association of the first and the second subunit of the Fc domains.

57. The pair of antigen binding molecules according to any one of embodiments 49 to 56, wherein the Fc domains of the first and second antigen binding molecules comprise one or more amino acid substitution that reduces binding to an Fc receptor and/or effector function.

58. The pair of antigen binding molecules according to any one of embodiments 49 to 57, wherein the cytokine receptor-binding domains are fused via peptide linkers to their respective fusion points.

59. The pair of antigen binding molecules according to embodiments 58, wherein the peptide linkers comprise an amino acid sequence of SEQ ID NO: 280 or SEQ ID NO: 281.

60. The pair of antigen binding molecules according to embodiments 49, wherein, a) the first antigen binding molecule comprises a first polypeptide comprising an amino acid sequence of SEQ ID NO: 267, a second polypeptide comprising an amino acid sequence of SEQ ID NO: 28, a third polypeptide comprising an amino acid sequence of SEQ ID NO: 265 and a fourth polypeptide comprising an amino acid sequence of SEQ ID NO: 266, and the second antigen binding molecule comprises a first polypeptide comprising an amino acid sequence of SEQ ID NO: 272, a second polypeptide comprising an amino acid sequence of SEQ ID NO: 28, a third polypeptide comprising an amino acid sequence of SEQ ID NO: 270 and a fourth polypeptide comprising an amino acid sequence of SEQ ID NO: 271; or b) the first antigen binding molecule comprises a first polypeptide comprising an amino acid sequence of SEQ ID NO: 267, a second polypeptide comprising an amino acid sequence of SEQ ID NO: 28, a third polypeptide comprising an amino acid sequence of SEQ ID NO: 265 and a fourth polypeptide comprising an amino acid sequence of SEQ ID NO: 266, and the second antigen binding molecule comprises a first polypeptide comprising an amino acid sequence of SEQ ID NO: 274, a second polypeptide comprising an amino acid sequence of SEQ ID NO: 28, a third polypeptide comprising an amino acid sequence of SEQ ID NO: 270 and a fourth polypeptide comprising an amino acid sequence of SEQ ID NO: 273; or c) the first antigen binding molecule comprises a first polypeptide comprising an amino acid sequence of SEQ ID NO: 269, a second polypeptide comprising an amino acid sequence of SEQ ID NO: 28, a third polypeptide comprising an amino acid sequence of SEQ ID NO: 265 and a fourth polypeptide comprising an amino acid sequence of SEQ ID NO: 268, and the second antigen binding molecule comprises a first polypeptide comprising an amino acid sequence of SEQ ID NO: 268, a second polypeptide comprising an amino acid sequence of SEQ ID NO: 28, a third polypeptide comprising an amino acid sequence of SEQ ID NO: 270 and a fourth polypeptide comprising an amino acid sequence of SEQ ID NO: 271; or d) the first antigen binding molecule comprises a first polypeptide comprising an amino acid sequence of SEQ ID NO: 269, a second polypeptide comprising an amino acid sequence of SEQ ID NO: 28, a third polypeptide comprising an amino acid sequence of SEQ ID NO: 265 and a fourth polypeptide comprising an amino acid sequence of SEQ ID NO: 268, and the second antigen binding molecule comprises a first polypeptide comprising an amino acid sequence of SEQ ID NO: 274, a second polypeptide comprising an amino acid sequence of SEQ ID NO: 28, a third polypeptide comprising an amino acid sequence of SEQ ID NO: 270 and a fourth polypeptide comprising an amino acid sequence of SEQ ID NO: 273.

61. The pair of antigen binding molecules according to embodiments 51, wherein the first antigen binding molecule comprises a first polypeptide comprising an amino acid sequence of SEQ ID NO: 276, a second polypeptide comprising an amino acid sequence of SEQ ID NO: 28 and a third polypeptide comprising an amino acid sequence of SEQ ID NO: 275, and the second antigen binding molecule comprises a first polypeptide comprising an amino acid sequence of SEQ ID NO: 277, a second polypeptide comprising an amino acid sequence of SEQ ID NO: 28 and a third polypeptide comprising an amino acid sequence of SEQ ID NO: 278.

### EXAMPLES

### Example 1: Generation of Binders for IFNγ and IL-2 Receptor Subunits

### 1.1 Generation of Antigens for Immunization and Phage Display

For llama immunization, the extracellular domain (ECD) of human IFNγR1 subunit and the ECD of human IFNγR2 subunit were co-expressed as an Fc-tagged heterodimer based on the knobs-into-holes technology (FIG. 1A, P1AG0843, SEQ ID NO: 48 and SEQ ID NO: 49). Likewise, the ECD of human IL-2Rβ (CD122) subunit and human IL-2Rγ (common gamma chain; CD132) subunit were co-expressed as an Fc-tagged heterodimer (FIG. 1D, P1AE2657, SEQ ID NO: 80 and SEQ ID NO: 81). The C-terminus of IFNγR1 ECD was fused to Fc knob via a G₄SG₄ linker. An AviTag was included at the C-terminus of the Fc for site-specific biotinylation during co-expression with BirA biotin ligase. The IFNγR2 ECD containing C174S substitution was fused to Fc hole with a G₄SG₄ linker and the construct contained a C-terminal Twin-Strep-tag. The C-terminus of IL-2Rβ ECD was fused to Fc knob via a GAQ linker. An AviTag was included at the C-terminus of the Fc for site-specific biotinylation during co-expression with BirA biotin ligase. The IL-2Rγ ECD was C-terminally fused to Fc hole with a GAQ linker.

For phage display, monovalent IFNγR1 ECD fused to biotinylated Fc knob paired with empty Fc hole (FIG. 1B, P1AG1987, SEQ ID NO: 48 and SEQ ID NO: 50) and monovalent IFNγR2 ECD C174S fused to Fc hole paired with empty biotinylated Fc knob (FIG. 1C, P1AG6735, SEQ ID NO: 49 and SEQ ID NO: 51), as well as monovalent IL-2Rβ ECD fused to biotinylated Fc knob paired with empty Fc hole (FIG. 1E, P1AF1104, SEQ ID NO: 80 and SEQ ID NO: 82) and monovalent IL-2Rγ ECD fused to Fc hole paired with empty biotinylated Fc knob (FIG. 1F, P1AF1106, SEQ ID NO: 81 and SEQ ID NO: 51), were generated. All antigens were produced in the Expi293 mammalian expression system (Expi293™ Expression System Kit, Thermo Fisher Scientific, Cat. No: A14635) following manufacturer's recommendation. Supernatants were harvested by centrifugation, filtered and purified by Protein A affinity chromatography (Protein A MabSelect™ SuRe™, Cytiva, Cat. No: 17543803) followed by size exclusion chromatography (SEC) (HiLoad S200 16/600, Cytiva, Cat. No: 28989335) according to manufacturer's recommendation.

### 1.2 VHH Library Construction

The VHH library was constructed using ˜3×10⁸ peripheral blood mononuclear cells (PBMCs) from bleed 2 and ˜6.6×10⁸ PBMCs bleed 3 of a single llama. The library was provided in the TG1 E. coli strain encoded in a derivative of the pADL-23c phagemid vector (Antibody Design Labs, San Diego, CA), incorporated by approximately ˜1×10⁹ independent electroporation events with >95% insert efficiency. Libraries were provided as ˜3.2×10¹⁰ cfu/mL E. coli in 2×YT medium containing 20% glycerol.

### 1.3 Generation of VHH Phage Library

E. coli containing the VHH-encoded phagemid library were inoculated in 2×YT medium containing 100 μg/ml ampicillin and 1% glucose. Following infection with VCSM13 helper phage, E. coli were grown in 2×YT medium containing 100 μg/ml ampicillin and 50 μg/ml kanamycin. Following overnight incubation on a shaker at 30° C., cultures were harvested by centrifugation at 8000 rpm for 20 minutes and supernatants were collected. Phage were precipitated in 20% PEG/2.5 M NaCl solution and incubated on ice for 1 hour. Precipitated phage were centrifuged at 8000 rpm for 20 minutes and pellets were resuspended in ddH₂O followed by precipitation in PEG/NaCl solution for 1 hour on ice. Phage were centrifuged at 8000 rpm for 20 minutes and pellets were resuspended in PBS containing 20% glycerol.

### 1.4 Isolation of IFNγR1-Specific, IFNγR2-Specific, IL-2Rβ-Specific and IL-2Rγ-Specific VHHs by Phase Display

To selectively enrich for antigen-specific binders and prevent the enrichment of Fc-specific binders, Fc-competitive soluble selections were performed against human IFNγR1 antigen (P1AG1987), human IFNγR2 antigen (P1AG6735), human IL-2Rβ antigen (P1AF1104) and human IL-2Rγ antigen (P1AF1106), in the presence of human Fc (FIG. 1G, P1AD4290, SEQ ID NO: 52 and SEQ ID NO: 53). An aliquot of 1×10¹¹ phage from the llama-derived VHH library was used per antigen. Phage and magnetic streptavidin beads (Dynabeads; M-280 Streptavidin, Invitrogen, Cat. No: 11205D) were independently blocked with 1% BSA, 0.1% Tween-20 in phosphate-buffered saline (PBS) to reduce non-specific interactions. All incubations were performed for one hour at 20° C., unless otherwise stated. The biotinylated, Fc-tagged antigens were added at 100 nM concentration to blocked phage in the presence of excess amounts (500 nM) of non-biotinylated Fc (FIG. 2). Following incubation, pre-blocked Dynabeads were added to capture biotinylated antigens of interest and bound VHH-expressing phage. Fc-specific and non-specific phage were removed by 5-10 wash steps in PBS containing 0.1% Tween-20 followed by PBS using a KingFisher magnetic particle processor (Thermo Fisher Scientific). Antigen-specific phage were eluted in 800 μl of 100 mM trimethylamine (TEA) and neutralized by addition of 400 μl of 1 M Tris pH 7.4. Mid-logarithmic phase TGT E. coli in 2×YT medium were infected with eluted phage, followed by infection with VCSM13 helper phage. Following addition of 100 μg/ml ampicillin and 50 μg/ml kanamycin, infected E. coli were incubated overnight on a shaker at 30° C. Phage were precipitated with PEG/NaCl and used for the next round. Selections were performed for a total of 3 rounds using decreasing target antigen concentrations (100 nM, 50 nM, 25 nM) while soluble Fc concentrations remained constant (500 nM) (FIG. 2).

### 1.5 Verification of Target Specificity by ELISA

Round 3 enrichment libraries were screened for target specificity vs Fc specificity by ELISA. Individual VHH clones were expressed in 1 ml E. coli cultures in a 96-well plate and supernatants containing soluble VHH were screened for specificity by ELISA. Supernatants were transferred to neutravidin 96-well plates coated with biotinylated Fc (P1AE6073, SEQ ID NO: 51 and SEQ ID NO: 54) or target Fc-tagged biotinylated antigen (P1AG1987, P1AG6735, P1AF 1104 or P1AF 1106). Following incubation for 1 hour at 20° C., plates were washed 3 times with PBST and 3 times with PBS. HRP-conjugated anti-His antibody (Sigma, Cat. No: A7058) was added to wells at 1:2000 dilution and plates were incubated for 1 hour at room temperature. Plates were washed 3 times with PBST followed by 3 washes with PBS. Plates were developed by adding 1-Step Ultra TMB-ELISA substrate (Thermo Fisher Scientific, Cat. No: 34028) and reactions were quenched with sulfuric acid. Absorbance at 450 nm was measured using a Tecan infinite M1000 Pro and reference absorbance values were measured at 650 nm. Reference-subtracted absorbance values at 450 nm were represented as stacked bar charts for IFNγR1-specific VHHs (FIG. 3A), IFNγR2-specific VHHs (FIG. 3B), IL-2Rβ-specific VHHs (FIG. 3C) and IL-2Rγ-specific VHHs (FIG. 3D). ELISA data suggested >90% of clones were target-specific and VHH from round 3 enrichment libraries were subsequently cloned into bispecific heavy chain antibody formats.

### Example 2: Evaluation of Agonist Activity Towards IFNγ and IL-2 Receptors

2.1 Assembly of Enriched VHH Libraries into Heavy Chain Antibody Formats

IFNγR activation is achieved by IFNγ-mediated cross-linking of the IFNγR1 and IFNγR2 receptor chains. To mimic the assembly in an IFNγ-independent manner, a VHH domain with IFNγR1 specificity was paired with an IFNγR2-specific VHH by fusing to knob-into-hole Fc chains via a flexible linker generating IFNγR1-IFNγR2 bispecific heavy chain antibodies. IL-2R activation is achieved by IL2-mediated cross-linking of the IL-2Rβ and IL-2Rγ receptor chains. Thus, to mimic the assembly in an IL2-independent manner, a VHH domain with IL-2Rβ specificity was paired with an IL-2Rγ-specific VHH by fusing to knob-into-hole Fc chains via a flexible linker generating IL-2Rβ-IL-2Rγ bispecific heavy chain antibodies. To this end, the round 3 VHH library enriched for IFNγR1- or IL-2Rβ-specificity were cloned in bulk using Gibson assembly to achieve format conversion of VHH by fusing to Fc knob via a 5(G₄S) linker (FIG. 4A). In the same way, the round 3 IFNγR2- or IL-2Rγ-specific VHH library were fused to Fc hole via a 5(G₄S) linker using Gibson assembly (FIG. 4B). During the assembly, a suitable signal sequence and vector compatible with mammalian expression were included. Primers, PCR conditions and Gibson assembly reactions were defined as per New England Biolabs' NEBuilder HiFi DNA Assembly protocol (Cat. No: E5510). Following bacterial transformation, colonies were selected at random and five unique sequences from each specificity pool were selected. Using the Expi293 system, a 5×5 matrix was generated to express 25 unique knob-into-holes bispecific heavy chain antibodies comprising IFNγR1 and IFNγR2 VHH pairs and 25 unique knob-into-holes bispecific heavy chain antibodies comprising IL-2Rβ VHH and IL-2Rγ VHH pairs (FIG. 5). Expi293 cells were transiently transfected following manufacturer's recommendation (Expi293™ Expression System Kit, Thermo Fisher Scientific, Cat. No: A14635) and supernatants containing secreted soluble bispecific heavy chain antibodies were collected. The bispecific heavy chain antibodies were purified by Protein A resin and further characterized for functional activity using reporter cell assays.

### 2.2 Functional Characterization of Bispecific Heavy Chain Antibodies

The HEK Blue IFNγ reporter cell assay (Invivogen) was used to screen bispecific heavy chain antibodies comprising different IFNγR1 and IFNγR2 VHH pairs and identify IFNγR agonists. Whereas, the HEK Blue IL-2 reporter cell assay (Invivogen) was used to screen bispecific heavy chain antibodies comprising different IL-2Rβ and IL-2Rγ VHH pairs and identify IL-2R agonists. The in vitro HEK Blue IFNγ reporter cell system (Invivogen, Cat. No: hkb-ifng) reports IFNγR signaling via a STAT1-inducible secreted alkaline phosphatase (SEAP) system. While the in vitro HEK Blue IL-2 reporter cell (Invivogen, Cat. No: hkb-i12) recapitulates the human IL-2R signaling pathway via expression of the IL-2R chains (IL-2Rα, β and γ subunits) and effectors of the downstream signaling cascade (JAK3 and STAT5) and a STAT5-inducible secreted alkaline phosphatase (SEAP) reporter system. Upon addition of QuantiBlue substrate (Invivogen, Cat. No: rep-qbs), absorbance at 650 nm is measured, correlating with SEAP levels and IFNγR and IL-2R activity, respectively. In the initial screening, supernatants from the Expi293 5×5 expression matrix containing soluble IFNγR1-IFNγR2 or IL-2Rβ-IL-2Rγ bispecific heavy chain antibodies were incubated with HEK Blue IFNγ or HEK Blue IL-2 reporter cells, respectively. Following a 20 hour incubation, IFNγR and IL-2 activity was quantified by addition of QuantiBlue reagent (Invivogen, Cat. No: rep-qbs), according to the manufacturer's recommendation. The absorbance at 650 nm was measured, with higher absorbance levels indicating increased IFNγR or IL-2 signaling.

Of the 25 tested IFNγR1-IFNγR2 bispecific heavy chain antibodies (format depicted in FIG. 5), 14 showed IFNγR activity (FIG. 6A). Thus, 14 pairs of IFNγR1- and IFNγR2-VHH domains demonstrated IFNγ agonistic activity. Of the 25 tested IL-2Rβ-IL-2Rγ bispecific heavy chain antibodies, 9 pairs containing non-specific VHH domains (IL2R1_4 and IL2Rγ_1) did not induce IL-2R activity. The other 16 pairs, which were formed of functional IL-2Rβ and IL-2Rγ binders, all showed IL-2R agonism (FIG. 6B). Thus, 16 pairs of IL-2Rβ- and IL-2Rγ-VHH domains demonstrated IL-2 agonistic activity.

Next, purified agonistic IFNγR1-IFNγR2 bispecific heavy chain antibodies (P1AH1877-P1AH1890, see Table 1) were tested for dose-dependent IFNγR signaling and agonistic IL-2Rβ-IL-2Rγ bispecific heavy chain antibodies were tested for dose-dependent IL-2R signaling (see Table 2).

TABLE 1
IFNγR1-IFNγR2 bispecific heavy chain antibodies generated by the 5 ×
5 expression matrix showing IFNγ agonistic activity in FIG. 6A.

| | IFNγR2_1 | IFNγR2_2 | IFNγR2_3 | IFNγR2_4 | IFNγR2_5 |
|---|---|---|---|---|---|
| | SEQ ID NO: 15 | SEQ ID NO: 16 | SEQ ID NO: 17 | SEQ ID NO: 18 | SEQ ID NO: 19 |
| IFNγR1_1 | P1AH1877 | P1AH1878 | | P1AH1879 | |
| SEQ ID NO: 11 | | | | | |
| IFNγR1_2 | P1AH1880 | P1AH1881 | | P1AH1882 | |
| SEQ ID NO: 12 | | | | | |
| IFNγR1_3 | P1AH1883 | P1AH1884 | | P1AH1885 | |
| SEQ ID NO: 13 | | | | | |
| IFNγR1_4 | | | | | |
| SEQ ID NO: 55 | | | | | |
| IFNγR1_5 | P1AH1886 | P1AH1887 | P1AH1889 | P1AH1888 | P1AH1890 |
| SEQ ID NO: 14 | | | | | |

A concentration range spanning from 100 nM to 0.01 nM with 10-fold serial dilution steps was used with HEK Blue IFNγ cells according to the Invivogen HEK Blue protocol. Absorbance at 650 nm was measured and responses were plotted using GraphPad software (FIG. 7). Graphs were separated according to VHH clone with IFNγR1 specificity, with recombinant human IFNγ (recIFNγ; SEQ ID NO: 26) and anti-FAP IgG fused to IFNγ homodimer (P1AF3574; SEQ ID NO: 24 and SEQ ID NO: 25) serving as reference molecules. The variety of responses observed in all graphs, ranging from potent IFNγ mimetics to weak agonists, demonstrated an interplay between both the IFNγR1 and IFNγR2 binders. EC50 values were derived using GraphPad Prism software (FIG. 8), illustrating the breadth of agonistic activities and underlining the potential for VHH pairs to achieve potent yet tunable cytokine mimetics.

TABLE 2
IL-2Rβ-IL-2Rγ bispecific heavy chain antibodies generated by the
5 × 5 expression matrix showing IL-2R activity in FIG. 6B.

| | | | | |
|---|---|---|---|---|
| IL2Rγ_1 | IL2Rγ_2 | IL2Rγ_3 | IL2Rγ_4 | IL2Rγ_5 |
| SEQ ID NO: 75 | SEQ ID NO: 70 | SEQ ID NO: 71 | SEQ ID NO: 72 | SEQ ID NO: 73 |

| | | | | |
|---|---|---|---|---|
| IL2Rβ_1 | P1AH1165 | P1AH1166 | P1AH1167 | P1AH1168 |
| SEQ ID NO: 66 | | | | |
| IL2Rβ_2 | P1AH1169 | P1AH1170 | P1AH1171 | P1AH1172 |
| SEQ ID NO: 67 | | | | |
| IL2Rβ_3 | P1AH1173 | P1AH1174 | P1AH1175 | P1AH1176 |
| SEQ ID NO: 68 | | | | |
| IL2Rβ_4 | | | | |
| SEQ ID NO: 74 | | | | |
| IL2Rβ_5 | P1AH1177 | P1AH1179 | P1AH1179 | P1AH1180 |
| SEQ ID NO: 69 | | | | |

A concentration range of 20 nM, 0.8 nM and 0.032 nM was used with HER Blue IL-2 reporter cells according to the Invivogen HER Blue protocol. Agonistic activity of the IL-2 mimetics was compared with IL-2v fused to anti-PDT IgG (PDT-IL2v; P1AE4422, SEQ ID NO: 83, SEQ ID NO: 84 and SEQ ID NO: 85). The VHH pairs displayed potent IL-2R agonism in a dose-dependent manner (FIG. 9). Moreover, in contrast to the natural cytokine in which agonism is mediated by a single molecule, the use of a VHH pair to activate the receptor provided opportunities for conditionally active IL-2 mimetics via molecular split approaches.

To further characterize the agonistic activity of the 14 functional IFNγR1- and IFNγR2-VHH pairs, various tumor cell lines with previously identified responsiveness to IFNγ (MKN45, BxPC-3, COR-L105; data not shown) were selected to explore therapeutically relevant downstream effects of IFNγR signaling which may have the capacity to modulate the tumor microenvironment (TME). In the first instance, MHC-I and PD-L1 were selected as tumor cell surface biomarkers to characterize the TME-modulating potential of functional bispecific VHH pairs. MHC-I upregulation is a downstream response following IFNγR stimulation and may provide an increase in antigenicity on tumor cells, while PD-L1 upregulation is another downstream indicator of functional IFNγ signaling. To assess the capacity of VHH pairs to induce IFNγ signaling in vitro, tumor cells were incubated with the 14 previously tested heavy chain antibodies (P1AH1877-P1AH1890) in concentration series ranging from 100 nM to 0.1 nM. After 72 hours, MHC-I and PD-L1 expression levels were quantified by flow cytometry and data were analyzed using FlowJo software. Mean fluorescent intensity (MFI) values were blank-subtracted and normalized to recombinant IFNγ responses. Upregulation of MHC-I and PD-L1 was measured in three tumor cell types: MKN45 (FIG. 10A, 10B), BxPC-3 (FIG. 10C, 10D) and COR-L105 (FIG. 10E, 10F). A variety of responses was observed, corroborating the broad range of agonistic activities observed previously in HEK Blue IFNγ reporter cells. One notable observation was an often decreased PD-L1 expression compared to MHC-I expression induced by certain VHH pairs, suggesting a complexity in IFNγ signaling that may engage different pathways and highlighting the potential for agonistic VHH pairs to elicit a bias towards desired phenotypic effects.

The induction of IFNγR signaling via an agonistic IFNγR VHH pair and IL-2R signaling via an agonistic IL-2R VHH pair provided opportunities to engineer conditionally active IFNγ and IL-2 mimetics. As a starting point for a conditional IFNγR agonist, P1AH1884 was selected as a potent IFNγ mimetic (FIG. 8) with activity closely correlating with recombinant IFNγ responses across all tumor lines tested (FIG. 9). For a conditional IL-2R agonist P1AH1177 was selected.

The corresponding VHH pairs were further engineered to generate conditionally active agonists.

### Example 3: Engineering of Conditionally Active IFNγ and IL-2 Mimetics

### 3.1 FAP-Dependent Biparatopic Assembly of Split Dual-Targeted IFNγ Mimetics

To achieve conditional immunomodulation within an immune-deserted tumor microenvironment, cell surface markers reportedly expressed in non-inflamed tumor phenotypes were further explored. Fibroblast activation protein (FAP), a serine protease described with high expression in cancer-associated stromal tissue of immune-deserted tumors, was selected as a target to achieve cold tumor penetration. As a next step, a pair of agonistic VHH domains mimicking IFNγ were distributed onto two distinct molecules to inactivate the VHH pair, whilst incorporating a FAP-dependent assembly strategy to restore agonistic VHH activity in the presence of FAP (FIG. 11A). To this end, two proximal epitopes on the FAP antigen were selected and the corresponding non-competing anti-FAP binders were engineered to mediate assembly of an agonistic VHH pair. In the first instance, VHH domains of the P1AH1884 heavy chain antibody were fused to the N-terminus of the VH or VL domains of the anti-FAP binders via a flexible 5(G₄S) linker, resulting in 8 possible format configurations (FIG. 11B-I). To test for FAP-dependent IFNγR signaling, upregulation of MHC-I and PD-L1 was quantified in three FAP expression scenarios. First, the A549 lung adenocarcinoma cell line was used to test for untargeted, FAP-independent activity in a FAP-negative setting. Next, A549 cells engineered for ectopic FAP expression were used to characterize FAP-dependent activity in cis, while both FAP-positive and FAP-negative cell lines were differentially labelled and co-cultured to compare cis and trans activity of FAP-dependent IFNγ mimetics. Split dual-targeted IFNγ mimetics were serially diluted to span a concentration range from 10 nM to 1 pM. Recombinant IFNγ and the intact, parental VHH pair deficient in FAP targeting (P1AH1884, format depicted in FIG. 5) were used as reference molecules. Following a 72 h incubation in the three FAP expression scenarios, MHC-I and PD-L1 expression levels were quantified by flow cytometry. In the absence of FAP expression, MHC-I and PD-L1 upregulation was induced by reference molecules while none of the combinations of split dual-targeted molecules exhibited FAP-independent activity (FIGS. 12A and 12B). In the presence of FAP expression, both the recapitulation of a cis setting (FIGS. 12C and 12D) and the recapitulation of a trans setting (FIGS. 12E and 12F) showed FAP-specific activity. In particular, the cis setting underscored the impact of geometry to achieve potent activity, with minor format adjustments (i.e. swapping of fusion points from VH to VL on anti-FAP binders) translating into differences in IFNγR signaling. The P1AI0831+P1AI0066 pair provided the most potent FAP-dependent IFNγR activity. Interestingly, the geometrical constraints did not apply to the same degree in a trans scenario, with all format combinations displaying FAP-dependent activity.

To further explore format options and extend our understanding of the constraints imposed by molecular geometry, we designed additional sets of molecules in which the VHHs were further distanced from the FAP binding domains. The VHHs were fused to the N-terminus of the opposite Fc chain via a 3(G₄S) linker (FIG. 13A-D) or to the C-terminus of the same Fc chain via a 3(G₄S) linker (FIG. 13E-H). The sets were tested in vitro in the three FAP expression scenarios, as described previously. FAP-independent activity was observed with the reference molecules (recombinant IFNγ and P1AH1884, FIGS. 14A and 14B) while split dual-targeted combinations did not show IFNγR agonism except at high concentration for one combination (P1AI5306+P1AI5307; FIG. 14A) in the FAP-negative scenario. Next, FAP-dependent activity was tested in cells expressing FAP (cis targeting). MHC-I upregulation (FIG. 14C) and PD-L1 upregulation (FIG. 14D) was observed with the reference molecules and the P1AI5306+P1A15307 combination, but all other combinations remained inactive. Finally, the combinations were tested in a trans scenario, to assess activity on FAP-negative cells in the presence of FAP-expressing cells. All combinations showed FAP-dependent trans activity, inducing both MHC-1 (FIG. 14E) and PD-L1 upregulation (FIG. 14F). By disconnecting the VHHs from the FAP binding moiety, FAP-dependent cis activity was rarely observed due to the previously identified geometrical constraints imposed in a cis scenario. However, in a trans setting, all combinations displayed IFNγR agonism regardless of the format.

Moreover, FAP-dependent IFNγ agonists comprising different lengths of linker between the VHH and the fusion point were explored (concepts described in FIG. 15). The molecule P1AJ8572 was replaced with P1AI0187 due to a batch failure in production. the activity of IFNγ agonists was measured by MHC-I upregulation. Specifically, activity in an untargeted scenario (A549 FAP-negative cells) as well as in cis-targeted (IFNγ activity in A549 FAP-positive cells co-cultured with A549 FAP-negative cells) and trans-targeted (IFNγ activity in A549 FAP-negative cells co-cultured with A549 FAP-positive cells) scenarios were assessed. Briefly, for the untargeted condition 7′000 A549 FAP-negative per well were seeded, while for assessing cis and trans activity a combination of 3′500 A549 FAP-negative and 3′500 A549 FAP-positive cells per well were respectively seeded in a 96 well flat-bottom plate. After overnight incubation at 37° C. and 5% CO₂ to allow the cells to adhere, test compounds (four dilution series, starting concentration of 50′000 pM) were added to the assay plate. Samples were incubated at 37° C. and 5% CO₂ for 72 h before the assay readout using a MHC-I antibody from BioLegend clone W6/32 (Cat No: 311430) measured on a BD FACS Fortessa.

In the absence of FAP expression, MHC-I upregulation was induced by recombinant IFNγ and by the IFNγ agonistic heavy chain antibody (P1AH1884), while none of the combinations of split dual-targeted molecules exhibited FAP-independent activity (FIGS. 16A and 16D). In the cis targeting scenario, MHC-I upregulation was observed with recombinant IFNγ and the IFNγ agonistic heavy chain antibody, yet all combinations of split dual-targeted molecules remained inactive (FIG. 16B and E). Finally, in the trans scenario all combinations of FAP-dependent split IFNRγ agonists showed FAP-dependent trans activity upregulation of MHC-I (FIGS. 16C and 16F).

### 3.2 Functional Characterization of EGFR-Dependent Trans-Targeted IFNγR Agonists

To further extend the application for biparatopic assembly of cytokine receptor agonists, the concept was tested with IFNγR agonists and EGFR-targeted assembly. A pair of agonistic VHH domains mimicking IFNγ were distributed onto two separate molecules to inactivate the VHH pair, whilst incorporating an EGFR-dependent assembly via two non-competing anti-EGFR Fabs. The VHH domains of the P1AH1884 heavy chain antibody were fused to the N-terminus of the VH or VL domains of the anti-EGFR binders via a flexible 5(G₄S) linker, resulting in eight possible format configurations (P1AK5514−P1AK5521) which can be paired to produce eight assembly combinations (FIG. 17). The activity of the eight assembly combination was measured assessing the upregulation of the chemoattractant CXCL10 following 24 h stimulation (FIG. 18).

In order to assess the activity of the eight assembly combinations, the upregulation of chemoattractant CXCL10 was measured following 24 h incubation with U87 and PBMCs. Briefly, 20′000 U87 cells per well were seeded in a 96 well plate flat bottom. After overnight incubation at 37° C. and 5% CO₂ to allow the cells to adhere, 50 μL/well of PBMCs at 2×10⁶ PBMCs/well was added to the wells. Lastly, 50 μL/well of control medium or compound dilution was added to the wells. Samples were incubated at 37° C. and 5% CO₂ for 24 h before assay readout with the human IP10 R&D ELISA KIT (cat #DY266) accordingly to the manufacturer's instructions.

While the degree of upregulation varied between the EGFR-targeted IFNγ agonists, all pairs demonstrated upregulation of the chemoattractant CXCL10 (FIG. 18).

### 3.3 PD1-Dependent Biparatopic Assembly of Split Dual-Targeted IL-2 Mimetics

In a bid to maintain conditional agonistic activity whilst incorporating tumor selectivity, a strategy was devised to harness cell surface markers, reportedly enriched within the tumor environment, to scaffold the assembly of the IL-2 agonistic VHH domain pair. The aim was to bind two proximal epitopes on a single target using two non-competing anti-target binders (FIG. 19A). In the first instance, PD-1 was selected as the target for several reasons. First, upregulation of PD-1 is observed upon T cell activation, providing an opportunity to enhance selectivity towards the tumor-specific effector T cell population. Second, PD-L1-competitive anti-PD-1 binders would provide immune checkpoint inhibition mediated by blockade of the PD1/PD-L1 signaling cascade. Third, recent evidence highlighted the synergistic effects of IL-2R agonism combined with cis-targeted PD-1 blockade, resulting in the differentiation of ‘better effector’ T cells (Codarri Deak et al. Nature 610; 161-172 (2022)).

In this experiment, the potency and the cis-delivery of two pairs of IL-2 mimetics (FIGS. 19B and 19C; FIGS. 19D and 19E), as well as PD1-IL2v (FIG. 19G, P1AE4422) and a potent IL-2 agonistic heavy chain antibody (FIG. 19F, P1AH117) as reference molecules, were measured based on their IL-2R signaling by treating activated PD-1⁺ and PD-1⁻ (anti-PD1 pre-treated) CD4 T cells with increasing concentrations of molecules. The purpose was to determine the dependency of the PD-1-based IL-2R agonists on the PD1 expression of the T cells in order to deliver an IL-2R signaling.

For this purpose CD4 T cells were sorted from healthy donor PBMCs with CD4 beads (Miltenyi, Cat. No: 130-045-101) and activated for 3 days in presence of 1 μg/ml plate bound anti-CD3 (overnight pre-coated, clone OKT3, Cat. No: 317315, BioLegend) and 1 μg/ml of soluble anti-CD28 (clone CD28.2, Cat. No: 302923, BioLegend) antibodies to induce PD1 expression. Three days later, the cells were harvested and washed several times to remove endogenous cytokines and half of the cells were labelled with CTV (5 μM, 5 min at 20° C.; Cat. No: C34557, Thermo Scientific) and the other half left unlabelled.

Then, the unlabelled cells were incubated with a saturating concentration of a competing anti-PD-1 antibody (in-house molecule, 10 μg/ml) for 30 min at 20° C. followed by several washing steps to remove the excess unbound anti-PD1 antibody. Thereafter the PD1 pre-blocked unlabelled cells (25 μl, 6×10⁶ cells/ml) were co-cultured 1:1 with the PD1⁺ CTV labelled cells (25 μl, 6×10⁶ cells/ml) in a V-bottom plate before being treated for 15 or 60 min at 37° C. with increasing concentrations of treatment molecules (50 μl, 1:10 dilution steps). To preserve the phosphorylation state, an equal amount of Phosphoflow Fix Buffer I (100 μl, Cat. No: 557870, BD) was added after incubation with the various treatment molecules. The cells were then incubated for an additional 30 min at 37° C. before being permeabilized overnight at −80° C. with Phosphoflow PermBuffer III (Cat. No: 558050, BD). On the next day STAT-5 in its phosphorylated form was stained for 30 min at 4° C. by using an anti-STAT-5P antibody (47/Stat5(pY694) clone, Cat. No: 562076, BD).

The cells were acquired at the FACS BD-LSR Fortessa (BD Bioscience). The frequency of STAT-5P was determined with FlowJo (V10) and plotted with GraphPad Prism.

The dose-response curves on PD-1⁺ T cells provide information on the potency of the PD1-based IL-2R agonists in signaling through the IL-2R. In addition, the dose-response curves on T cells pre-treated with a competing anti-PD1 antibody, to prevent the PD1 mediated delivery, show the potency of the PD1-based IL-2R agonist molecules in providing IL-2R signaling independently from PD-1 expression. PD1-IL2v showed reduced activity on T cells in absence of PD1 binding (pre-blocked) (FIG. 20, black solid and black dashed lines). While no activity was seen for the two combinations of split IL-2 mimetics (P1AH6850+P1AH6813, P1AH6814+P1AI1593) in absence of PD1 binding (FIG. 20, light grey dashed lines). No reduction in IL-2 signaling was seen for the non-targeted IL-2 agonistic bispecific heavy chain antibody (P1AH1177, FIG. 20, dark gray solid and dark grey dashed lines).

In order the study the biological activity of PD-1 targeted IL-2R agonists downstream of STAT-5 phosphorylation, the molecules of interest were tested in a PBMC proliferation and activation assay. Briefly, human PBMCs from healthy donors were isolated from buffy coats (Zurich Blood Donation Center in accordance with the Declaration of Helsinki) using standard density-gradient isolation over Lymphoprep™ (STEMCELL Technologies). Freshly isolated PBMCs were stained with CellTrace™ Violet Cell proliferation Kit (Thermo Fischer, Cat. No. C34557). 200,000 CTV labelled PBMCs per well were added to U-bottom 96-well plate (TPP) and co-incubated with test compounds at 5 concentrations ranging from 50,000 to 5 pM in assay medium (RPMI 1640 (Gibco) containing 10% FBS (Gibco) and 1×Glutamax (Gibco)). Assay plates were incubated at 37° C. and 5% CO₂ for five days before flow cytometry readout. Cells were stained with Zombie Aqua™ Fixable Viability Kit (Biolegend, Cat. No. 423102) in PBS containing Human TruStain FcX™ (Biolegend, Cat. No. 422302) for 10 minutes at room temperature. Cells were washed with FACS buffer (PBS containing 0.1% BSA) and stained with fluorescent dye-conjugated antibodies against human CD4 (clone SK3, BD, Cat. No 563550), PD-1 (clone EH12.1, BD, Cat. No 612791), CD56 (clone HCD56, Biolegend, Cat. No 318334), CD122 (clone 5H4, Biolegend, Cat. No 339006), γδ TCR (clone 11F2, BD, Cat. No 655410), CD25 (clone BC96, Biolegend, Cat. No 302610), CD3 (clone OKT3, Biolegend, Cat. No 317340) and CD8 (clone SKI, BD, Cat. No 560179) in FACS buffer for 30 minutes at 4° C. After two washing steps, cells were resuspended in FACS buffer and acquired with a 5-laser A3 Symphony instrument (BD). Data analysis was performed with FlowJo v10.8.1 and Prism 8 (GraphPad software).

At baseline, different cell types were identified according to lineage marker expression on the cell surface by flow cytometry (FIG. 21). Since the goal is to generate PD-1 targeted IL-2R agonists that specifically deliver IL-2 signals to PD-1 expressing cells while sparring PD-1 negative cells, the prevalence of PD-1⁺ IL-2R⁺ target cells was evaluated within the PBMC fraction. Across the two tested donors, a subset of CD8 T cells and γδ T cells (10-25%) co-expressed PD-1 and IL-2Rβ (FIG. 21D and E) whereas NK cells expressed high levels of IL-2Rβ and almost no PD-1 (FIG. 21F). Upregulation of the activation marker CD25 and the proliferative capacity of several cell types were used as functionality readouts at assay endpoint. An untargeted IL-2 agonistic heavy chain antibody (Fc-VHH; P1AH1177) showed a strong CD25 expression on a large fraction of CD8 T cells (FIG. 22A), γδ T cells (FIG. 22B) and NK cells (FIG. 22C), confirming the strong potency of IL-2R agonists. PD-1 targeted IL-2R agonist (P1AH6814+P1AI1593) demonstrated a dose-dependent induction of CD25 expression on a subset of CD8 T cells (FIG. 22A) and γδ T cells (FIG. 22B) but not on NK cells (FIG. 22C). A similar pattern was observed with the frequency of proliferating cells (FIG. 23) highlighting that the biparatopic assembly of IL-2R agonist onto PD-1 ensures the specific targeting of PD-1 expressing cells. PD-1 targeted IL-2R agonist (P1AH6814+P1AI1593) showed no activity on the phenotype and proliferation of NK cells with high IL-2R expression and negligible PD-1. The PD-1 targeted IL-2R agonist (P1AH6814+P1AI1593) was further tested in the human breast BC004 patient derived cells orthotopically injected into humanized BRGS47 mice. BC004 PDX material (human breast carcinoma) were originally obtained from OncoTest (Freiburg, Germany) and after in vivo expansion deposited in the Roche-Glycart internal cell bank. Tumor fragments were digested with Collagenase D and DNase I (Roche, Switzerland), and BC004 cells were used for transplantation. 1×10⁶ cells per animal were injected intra mammary fat pad in 100 μl of RPMI cell culture medium (Gibco, Germany) into the flank of mice using a 1 ml tuberculin syringe (BD Biosciences, Germany).

Fully humanized BRGS47 female mice (Jackson Labs, USA) were maintained under specific-pathogen-free condition with daily cycles of 12 h light/12 h darkness according to committed guidelines (GV-Solas; Felasa; TierschG). Continuous health monitoring was carried out on a regular basis.

Mice were injected intra mammary fat pad on study day 0 with 1×10⁶ of BC004 cells, randomized and weighed. Forty days after the tumor cell injection (tumor volume >200 mm³), mice were injected i.v. with the following molecules: P1AI1593, P1AH6814, Pembrolizumab 1 mg/kg, PD1-IL2v 0.1 mg/kg and FOLR1-TCB 0.3 mg/kg or Vehicle once a week for four weeks. The PD-1 targeted IL-2R agonist split constructs P1AH6814 and P1AI1593 were given once a week but on consecutive days. Groups of mice were also tested with Vehicle or FolR1-TCB as single agent or the combination of the PD-1 targeted IL-2R agonist, PD1-IL2v or Pembrolizumab with FOLR1-TCB. Due to the chimeric nature of the BRGS47 humanized mice, no HLA-restricted T cell responses occur in the BC004 breast orthotopic model. In order to see anti-tumor response in this mouse study the test compounds needed to be combined with the bispecific FOLR1-TCB molecule that triggers signal one first and as such allows the evaluation of the additive effect of immunomodulator or checkpoint inhibitor compounds. All mice were injected i.v. with 200 μl of the appropriate solution. The mice in the Vehicle group were injected with Histidine Buffer and the treatment groups with the different constructs. To obtain the proper amount of immuno-conjugate per 200 μl, the stock solutions were diluted with Histidine Buffer when necessary (see Table 3). Tumor growth measurements were evaluated with a caliper three times a week and plotted with GrahPad Prism software as volume in mm³+/−SEM.

FIG. 24 shows that the combination FOLR1-TCB 0.3 mg/kg+PD1-IL2v mimetic (P1AI1593 day 1+P1AH6814 day 2) mediated superior efficacy in terms of tumor growth inhibition compared to FOLR1-TCB single agent treatment and the other combination groups: FOLR1-TCB 0.3 mg/kg+PD1-IL2v and FOLR1-TCB 0.3 mg/kg+Pembrolizumab.

TABLE 3
Overview of administered compounds.

| | | | | Concentration |
|---|---|---|---|---|
| Compound | Dose | | ID | (mg/mL) |
| FOLR1-TCB | 0.3 | mg/kg | P1AK1120 | 3.75 (=stock solution) |
| IL-2 receptor | 1 | mg/kg | P1AI1593 | 5.89 (=stock solution) |
| agonist | | | | |
| (IL-2Rβ VHH) | | | | |
| IL-2 receptor | 1 | mg/kg | P1AH6814 | 2.14 (=stock solution) |
| agonist | | | | |
| (IL-2Rγ VHH) | | | | |
| PD1-IL2v | 0.1 | mg/kg | P1AD5438 | 3.16 (=stock solution) |
| Pembrolizumab | 1 | mg/kg | Keytruda | 25 (=stock solution) |

### 3.4 Functional Characterization of Further Cis-Targeted IL-2R Agonists

Further investigations of the biparatopic assembly concept were undertaken with alternative anti-PD-1 Fab binders (FIG. 25). HEK Blue IL-2 wt cells (PD-1-negative) and HEK Blue IL-2 cells expressing human PD-1 were used to assess IL-2 signaling. As observed with the previously described PD-1-targeted IL-2R agonists (P1AH6814+P1AI1593 and P1AH6850+P1AH6813), none of the tested combinations showed IL-2R signaling in the absence of PD-1 expression (FIG. 26A) whereas replacing PD-1 binder 0376 with 7G12 (P1AK2802+P1AK2599) also demonstrated strong potency on PD-1 expressing cells (FIG. 26B). Of the eight tested PD-1-targeted IL-2R agonists containing the same set of PD-1 binders (7G12 and 1040), three demonstrated strong dose-response IL-2R activation, highlighting the impact of the geometry on biparatopic assembly and delivery of IL-2 signals.

To further explore the biparatopic assembly of cis-targeted IL-2R agonists, the concept was tested with LAG3-targeting constructs, as another example besides PD-1 targeting of T cell in cis. Two non-competing LAG3-specific Fabs were selected and engineered to mediate assembly of the agonistic VHH pair. VHH domains of the P1AH1177 heavy chain antibody were fused to the N-terminus of the VH or VL domains of the anti-LAG3 binders via a flexible 5(G4S) linker, resulting in eight possible format configurations (P1AJ5654−P1AJ5661) which can be paired to produce eight assembly combinations (FIG. 27). In order to assess the cis activity of IL-2R agonists targeted to LAG3, the phosphorylation of STAT5 on pre-activated CD4 T cells after 60 min incubation with test compounds was determined as previously described. Of note, the activation protocol yields comparable frequencies of PD-1 and LAG3 expressing T cells (data not shown). One pair of LAG3-targeted IL-2R agonists (P1AJ5658+P1AJ5657) led to IL-2R signaling (FIGS. 28A and 28B).

### 3.5 Functional Characterization of Trans-Targeted IL-2R Agonists

To further explore the biparatopic assembly of IL-2R agonists, the concept was tested with tumor (EGFR, HER2, HER3) or stroma (FAP) targets to measure delivery of IL-2R signaling in a trans setting. The pair of agonistic VHH domains of heavy chain antibody P1AH1177 mimicking IL-2 were distributed onto two distinct molecules to inactivate the VHH pair, whilst incorporating a target-dependent assembly strategy to restore agonistic VHH activity in the presence of each target (FAP, EGFR, HER2, HER3). Two non-competing target-specific Fabs were selected for each target and engineered to mediate assembly of the agonistic VHH pair. In the first instance, VHH domains of the P1AH1177 heavy chain antibody were fused to the N-terminus of the VH or VL domains of the anti-FAP binders via a flexible 5(G4S) linker, resulting in eight possible format configurations (P1AJ5092−P1AJ5099) which can be paired to produce eight assembly combinations (FIG. 29). In addition, molecular concepts targeting EGFR were designed with two non-competing anti-EGFR Fabs. The eight possible format configurations (P1AJ4165−P1AJ4172) can be paired to produce eight assembly combinations (FIG. 31). Moreover, two pairs of Her2-targeted IL-2R agonists (P1AJ4174+P1AJ4175 and P1AJ4173+P1AJ4176) as well as two pairs of Her3-targeted IL-2R agonists (P1AJ4178+P1AJ4179 and P1AJ4177+P1AJ4180) were analyzed (FIG. 33).

To measure the capacity of FAP, EGFR, HER2 and HER3 targeted IL-2R agonists to induce IL-2R signaling, a co-culture system of target positive or negative cell lines and IL-2Rβγ Bioassay cells (Promega, CS2018K02) was used. IL-2Rβγ Bioassay cells express physiological levels of IL-2Rβ and IL-2Rγ similar to human T cells, with a STAT5 phosphorylation luminescent reporter system. Briefly, 10,000 target cells (A549 FAP negative, A549 FAP positive, A-431 EGFR high, OE19 EGFR medium, LoVo EGFR medium, MDA-MB-231 HER2/HER3 negative or OE19 HER2/HER3 high) per well were seeded in a white 384-well flat bottom tissue culture treated plate (Falcon, Cat. No. 353988). After 5-6 h of incubation at 37° C. and 5% CO₂ to allow the cells to adhere, test compounds (4-fold dilution series, starting concentration of 100 nM) and 10,000 IL-2Rβγ Bioassay reporter cells per well were added to the assay plate. Samples were incubated at 37° C. and 5% CO₂ for 20 h before assay readout with Bio-Glo-NL Luciferase Assay System (Promega, Cat. No. J3081) according to the manufacturer's instructions. Luminescence readout of luciferase activity was performed using Tecan Spark TOM plate reader (500 ms acquisition time). Dose-response of test compounds vs fold change IL-2R signaling (RLU value/background) were plotted in GraphPad Prism software.

While the activity of PD1-IL2v was comparable in the presence and absence of FAP on A549 target cells, there was a clear activity gain of FAP-IL2v in A549 FAP+ cells compared to FAP-cells, demonstrating the validity of the experimental conditions to study trans IL-2R signaling (FIG. 30). A bell-shaped trans activity of FAP-targeted IL-2R agonists was observed, with the strongest IL-2R signaling for the pairs that contain one VHH fused to the heavy chain of a FAP binder and the other VHH fused to the light chain of the second FAP binder (e.g P1AJ5098+P1AJ5093, FIG. 30B). Importantly, there was no activity of the FAP-targeted IL-2R agonists in the absence of FAP (FIG. 30A), highlighting the biparatopic assembly requirement for IL-2R signaling.

A similar experimental setup was applied to characterize the activity of EGFR-targeted IL-2R agonists. EGFR expression was assessed by flow cytometry using a PE-conjugated anti-human EGFR antibody (Biolegend, Cat. No. 352904) on A-431, LoVo and OE19 target cell lines (FIG. 32A). Of the eight tested EGFR-targeted IL-2R agonists, one pair (P1AJ4165+P1AJ4172) showed consistent activity in the presence of medium or high EGFR expressing target cells (FIG. 32B, 32C and 32D). High target expression seems to be required for IL-2R signaling in a trans setting. Moreover, only one HER2-targeted IL-2R agonist (P1AJ4174+P1AJ4175) displayed a weak ability to trigger IL-2R signaling in the presence of OE19 target cells expressing high levels of HER2 (FIG. 34B).

### 3.6 Evaluation of Different Antibody Fragments as Target Binding Domains or Cytokine Receptor Binding Domains

In order to understand if the small size of single domain antibodies would be favorable for optimal assembly of IL-2R agonists, we tested four anti-PD-1 VHH binders as target-binding domains as shown in FIG. 35. None of the tested combinations showed IL-2R signaling in the absence of PD-1 expression (FIG. 36A). While the six tested combinations demonstrated IL-2R signaling in reporter assays (FIG. 36B), the magnitude of the response was weaker than with anti-PD-1 Fabs (P1AH6814+P1AI1593).

In order to understand if the small size of single domain antibodies (VHH) as cytokine receptor-binding domains is beneficial for optimal IL-2R signaling, four pairs of anti-IL2R Fabs (FIG. 37A-D) and one pair of anti-IL2R scFv (FIG. 37E and F) using the biparatopic assembly concept were tested in a HEK Blue IL-2 reporter assay.

While none of the tested combinations showed activity in the absence of PD-1 expression (FIG. 38A), they demonstrated varying levels of activity in PD-1 expressing reporter cells (FIG. 38B). The strongest IL-2R signaling was observed with anti-IL2R agonists comprising VHH domains (P1AH6814+P1AI1593) followed by the anti-IL2R agonists comprising scFv domains (P1AL9310+1AL9311). Anti-IL2R Fab agonists displayed the weakest potency, indicating that VHH-based agonists may be ideal candidates to bring IL2Rβ and IL2Rγ receptor chains in close proximity and trigger IL-2R signaling.

### 3.7 Role of Distance and Geometry in Biparatopic Assembly

To further investigate the role of geometry in biparatopic assembly of cytokine mimetics, available x-ray crystal structures comprising Fab:target complexes were used to measure the distance between N-terminal residues of two non-competing anti-target Fabs. Distances were measured for PD-1 (Table 4), EGFR (Table 5), HER2 (Table 6) and HER3 (Table 7) targets.

TABLE 4
Distances (Å) measured between the N-termini of the
heavy chain (HC) and light chain (LC) of two anti-PD-1
Fabs. The distance between the most proximal combination
of N-terminal fusion points is highlighted in grey.

| Distance (Å) | PD-1 binder 2 HC | PD-1 binder 2 LC |
|---|---|---|
| PD-1 binder 1 HC | 63 | 42 |
| PD-1 binder 1 LC | 35 | 36 |

TABLE 5
Distances (Å) measured between the N-termini of the
heavy chain (HC) and light chain (LC) of two anti-EGFR
Fabs. The distance between the most proximal combination
of N-terminal fusion points is highlighted in grey.

| Distance (Å) | EGFR binder 2 HC | EGFR binder 2 LC |
|---|---|---|
| EGFR binder 1 HC | 59 | 48 |
| EGFR binder 1 LC | 33 | 45 |

TABLE 6
Distances (Å) measured between the N-termini of the
heavy chain (HC) and light chain (LC) of two anti-HER2
Fabs. The distance between the most proximal combination
of N-terminal fusion points is highlighted in grey.

| Distance (Å) | Her2 binder 2 HC | Her2 binder 2 LC |
|---|---|---|
| Her2 binder 1 HC | 58 | 50 |
| Her2 binder 1 LC | 67 | 64 |

TABLE 7
Distances (Å) measured between the N-termini of the
heavy chain (HC) and light chain (LC) of two anti-HER3
Fabs. The distance between the most proximal combination
of N-terminal fusion points is highlighted in grey.

| Distance (Å) | Her3 binder 2 HC | Her3 binder 2 LC |
|---|---|---|
| Her3 binder 1 HC | 92 | 71 |
| Her3 binder 1 LC | 94 | 83 |

The measurements confirmed the impact of geometry in targeted biparatopic assembly of cytokine mimetics. In conclusion, the shorter the distance between two non-competing anti-target binders, the greater the potency of the resulting mimetic assembly.

### Example 4. Identification of IL-7R Agonists

### 4.1 Generation of Antigen for Immunization and Phage Display

For alpaca immunization and phage display, the extracellular domain (ECD) of human IL-7Rα (CD127) subunit was expressed as an Fc-tagged monomer based on the knobs-into-holes technology (FIG. 39A, P1AI1009, SEQ ID NO: 208 and SEQ ID NO: 209). The C-terminus of IL-7Rα ECD was fused to Fc knob via an ASGS linker. An AviTag was included at the C-terminus of the Fc for site-specific biotinylation during co-expression with BirA biotin ligase. All antigens were produced in the Expi293 mammalian expression system (Expi293™ Expression System Kit, Thermo Fisher Scientific, Cat. No: A14635) following manufacturer's recommendation. Supernatants were harvested by centrifugation, filtered and purified by Protein A affinity chromatography (Protein A MabSelect™ SuRe™ 15, Cytiva, Cat. No: 17543803) followed by size exclusion chromatography (SEC) (HiLoad S200 16/600, Cytiva, Cat. No: 28989335) according to the manufacturer's recommendation.

### 4.2. VHH Library Construction

The VHH libraries were constructed using lymphocytes isolated from two alpacas. The libraries were provided in the TG1 E. coli strain encoded in a derivative of the pHEN4 phagemid vector (Hassanzadeh-Ghassabeh et al., 2010), yielding 5-7×10⁸ clones per library. Libraries were provided in 2×YT medium containing 20% glycerol.

### 4.3 Generation of VHH Phage Library

E. coli containing the VHH-encoded phagemid library were inoculated in 2×YT medium containing 100 μg/ml ampicillin and 1% glucose. Following infection with VCSM13 helper phage, E. coli were grown in 2×YT medium containing 100 μg/ml ampicillin and 50 μg/ml kanamycin. Following overnight incubation on a shaker at 30° C., cultures were harvested by centrifugation at 8000 rpm for 20 minutes and supernatants were collected. Phage were precipitated in 20% PEG/2.5 M NaCl solution and incubated on ice for 1 hour. Precipitated phage were centrifuged at 8000 rpm for 20 minutes and pellets were resuspended in ddH2O followed by precipitation in PEG/NaCl solution for 1 hour on ice. Phage were centrifuged at 8000 rpm for 20 minutes and pellets were resuspended in PBS containing 20% glycerol.

### 4.4 Isolation of IL-7Rα-Specific VHHs by Phage Display

To selectively enrich antigen-specific binders and prevent the enrichment of Fc-specific binders, Fc competitive soluble selections were performed against human IL-7Rα antigen (P1A11009) in the presence of human Fc (FIG. 39B, P1AD4290). An aliquot of 1×10¹² phage from each of the two alpaca-derived VHH libraries was used per target antigen. Phage and magnetic streptavidin beads (Dynabeads; M-280 Streptavidin, Invitrogen, Cat. No: 11205D) were independently blocked with 1% BSA, 0.1% Tween-20 in phosphate-buffered saline (PBS) to reduce non-specific interactions. All incubations were performed for one hour at 20° C., unless otherwise stated. The biotinylated, Fc-tagged IL-7Rα antigen was added at 100 nM concentration to blocked phage in the presence of excess amounts (500 nM) of non-biotinylated Fc (FIG. 39C). Following incubation, pre-blocked Dynabeads were added to capture biotinylated antigen of interest and bound VHH-expressing phage. Fc-specific and non-specific phage were removed by 5-10 wash steps in PBS containing 0.1% Tween-20 followed by PBS using a KingFisher magnetic particle processor (Thermo Fisher Scientific). Antigen-specific phage were eluted in 800 μl of 100 mM trimethylamine (TEA) and neutralized by addition of 400 μl of 1 M Tris pH 7.4. Mid-logarithmic phase TG1 E. coli in 2×YT medium were infected with eluted phage, followed by infection with VCSM13 helper phage. Following addition of 100 μg/ml ampicillin and 50 μg/ml kanamycin, infected E. coli were incubated overnight on a shaker at 30° C. Phage were precipitated with PEG/NaCl and used for the next round. Selections with alpaca library 1 and alpaca library 2 were performed for a total of four rounds using decreasing target antigen concentrations (100 nM, 50 nM, 25 nM, 12.5 nM) while soluble Fc concentrations remained constant (500 nM) (FIG. 39C).

### 4.5 Verification of Target Specificity by ELISA

Round 3 and round 4 enrichment libraries were screened for target specificity vs Fc specificity by ELISA. Individual VHH clones were expressed in 1 ml E. coli cultures in a 96-well plate and supernatants containing soluble VHH were screened for specificity by ELISA. Supernatants were transferred to neutravidin 96-well plates coated with biotinylated Fc (P1AE6073) or target Fc-tagged biotinylated antigen (P1AI1009). Following incubation for 1 hour at 20° C., plates were washed 3 times with PBST and 3 times with PBS. HRP-conjugated anti-His antibody (Sigma, Cat. No: A7058) was added to wells at 1:2000 dilution and plates were incubated for 1 hour at 20° C. Plates were washed 3 times with PBST followed by 3 washes with PBS. Plates were developed by adding 1-Step Ultra TMB-ELISA substrate (Thermo Fisher Scientific, Cat. No: 34028) and reactions were quenched with sulfuric acid. Absorbance at 450 nm was measured using a Tecan infinite M1000 Pro and reference absorbance values were measured at 650 nm. Reference-subtracted absorbance values at 450 nm were represented as stacked bar charts for round 3 library 1 and library 2 (FIG. 40A and B, respectively) and round 4 library 1 and library 2 (FIG. 40C and D, respectively). ELISA data suggested that library 2 showed a greater enrichment of target-specific clones. 16 target-specific VHH were selected and subsequently cloned into bispecific heavy chain antibody formats.

4.6 Format Conversion of VHH into Bispecific Heavy Chain Antibodies

IL-7R activation is achieved by IL7-mediated cross-linking of the IL-7Rα (CD127) and IL-2Rγ (CD132; common gamma chain) receptor chains. To mimic receptor assembly in the absence of IL-7, an IL-7Rα-specific VHH domain was paired with previously identified VHH domains with IL-2Rγ specificity (IL-2Rγ 2, IL-2Rγ_3, IL-2Rγ_4 and IL-2Rγ_5). VHH were fused to knob-into-hole Fc chains via a flexible linker generating IL-7Rα-IL-2Rγ bispecific heavy chain antibodies. Sixteen IL-7Rα-specific VHH were selected from rounds 3 and 4 of phage display enrichment and four previously identified IL-2Rγ-specific VHH were selected. Format conversion was performed by Gibson assembly cloning by fusing IL-7Rα-specific VHH to Fc knob via a 5(G4S) linker (FIG. 41A) and by fusing IL-2Rγ-specific VHH to Fc hole via a 5(G4S) linker (FIG. 41B). During the assembly, a suitable signal sequence and vector compatible with mammalian expression were included. Primers, PCR conditions and Gibson assembly reactions were defined as per New England Biolabs' NEBuilder HiFi DNA Assembly protocol (Cat. No: E5510). Next, the Expi293 system was used to express bispecific heavy chain antibodies in a 16×4 matrix to generate 64 unique antibodies comprising IL-7Rα VHH and IL-2Rγ VHH pairs (FIG. 41C). Expi293 cells were transiently transfected following manufacturer's recommendation (Expi293™ Expression System Kit, Thermo Fisher Scientific, Cat. No: A14635) and supernatants containing secreted soluble bispecific heavy chain antibodies were collected. The bispecific heavy chain antibodies were purified by Protein A resin and further characterized for functional activity using reporter cell assays.

TABLE 8
IL-7Rα-IL-2Rγ bispecific heavy chain antibodies
generated by the 16 × 4 expression matrix.

| | IL2Rγ_2 | IL2Rγ_3 | IL2Rγ_4 | IL2Rγ_5 |
|---|---|---|---|---|
| | SEQ ID NO: 226 | SEQ ID NO: 227 | SEQ ID NO: 228 | SEQ ID NO: 229 |
| IL-7Rα_1 | P1AL0466 | P1AL0482 | P1AL0498 | P1AL0514 |
| SEQ ID NO: 210 | | | | |
| IL-7Rα_2 | P1AL0467 | P1AL0483 | P1AL0499 | P1AL0515 |
| SEQ ID NO: 211 | | | | |
| IL-7Rα_3 | P1AL0468 | P1AL0484 | P1AL0500 | P1AL0516 |
| SEQ ID NO: 212 | | | | |
| IL-7Rα_4 | P1AL0469 | P1AL0485 | P1AL0501 | P1AL0517 |
| SEQ ID NO: 213 | | | | |
| IL-7Rα_5 | P1AL0470 | P1AL0486 | P1AL0502 | P1AL0518 |
| SEQ ID NO: 214 | | | | |
| IL-7Rα_6 | P1AL0471 | P1AL0487 | P1AL0503 | P1AL0519 |
| SEQ ID NO: 215 | | | | |
| IL-7Rα_7 | P1AL0472 | P1AL0488 | P1AL0504 | P1AL0520 |
| SEQ ID NO: 216 | | | | |
| IL-7Rα_8 | P1AL0473 | P1AL0489 | P1AL0505 | P1AL0521 |
| SEQ ID NO: 217 | | | | |
| IL-7Rα_9 | P1AL0474 | P1AL0490 | P1AL0506 | P1AL0522 |
| SEQ ID NO: 218 | | | | |
| IL-7Rα_10 | P1AL0475 | P1AL0491 | P1AL0507 | P1AL0523 |
| SEQ ID NO: 219 | | | | |
| IL-7Rα_11 | P1AL0476 | P1AL0492 | P1AL0508 | P1AL0524 |
| SEQ ID NO: 220 | | | | |
| IL-7Rα_12 | P1AL0477 | P1AL0493 | P1AL0509 | P1AL0525 |
| SEQ ID NO: 221 | | | | |
| IL-7Rα_13 | P1AL0478 | P1AL0494 | P1AL0510 | P1AL0526 |
| SEQ ID NO: 222 | | | | |
| IL-7Rα_14 | P1AL0479 | P1AL0495 | P1AL0511 | P1AL0527 |
| SEQ ID NO: 223 | | | | |
| IL-7Rα_15 | P1AL0480 | P1AL0496 | P1AL0512 | P1AL0528 |
| SEQ ID NO: 224 | | | | |
| IL-7Rα_16 | P1AL0481 | P1AL0497 | P1AL0513 | P1AL0529 |
| SEQ ID NO: 225 | | | | |

### 4.7 Functional Characterization of Bispecific Heavy Chain Antibodies

The HEK Blue IL-7 reporter cell assay (Invivogen) was used to screen bispecific heavy chain antibodies comprising different IL-7Rα and IL-2Rγ VHH pairs and identify IL-7R agonists. The in vitro HEK Blue IL-7 reporter cell (Invivogen, Cat. No: hkb-il7) recapitulates the human IL-7R signaling pathway via expression of the IL-7Rα and IL-2Rγ chains, effectors of the downstream signaling cascade (JAK3 and STAT5) and a STAT5-inducible secreted alkaline phosphatase (SEAP) reporter system. Following addition of QuantiBlue substrate (Invivogen, Cat. No: rep-qbs), absorbance at 650 nm is measured, correlating with SEAP levels and IL-7R activity. The 64 heavy chain antibodies and reference molecules were incubated with HEK Blue IL-7 cells for 20 hours. IL-7R activity was quantified by addition of QuantiBlue reagent, according to the manufacturer's recommendation. The absorbance at 650 nm was measured and fold response/background was plotted as bar charts (FIG. 42A-D). All 64 molecules showed IL-7R agonism with a broad range of activity. 12 agonistic VHH pairs (IL-7Rα_1, IL-7Rα_2, IL-7Rα_3, IL-7Rα_5, IL-7Rα_6, IL-7Rα_7, IL-7Rα_10, IL-7Rα_11, IL-7Rα_13, IL-7Rα_14, IL-7Rα_15, IL-7Rα_16 in combination with IL2Rγ_2) were selected to further engineer for PD-1-targeted biparatopic assembly.

### 4.8 Design of PD-1-Targeted Biparatopic Assembly of IL-7 Mimetics

In a bid to bias agonistic activity towards tumor-reactive T cells, the PD-1 cell surface marker was selected to scaffold the assembly of the IL-7 mimetic VHH pair. The aim was to bind two proximal epitopes on PD-1 using two non-competing anti-PD-1 binders to bridge the assembly of a functional IL-7 mimetic (FIG. 43A). VHH fusion points on the two PD-1 binders were selected according to N-terminal residue proximity of the two Fabs determined by x-ray crystal structures (Table 4). The 12 selected IL-7Rα-specific VHH were fused to the VL of anti-PD-1 binder 1 via a 5(G4S) linker (P1AL0544−P1AL0556, FIG. 43B) and the selected VHH domain with IL-2Rγ specificity was fused to the VH of anti-PD-1 binder 2 (P1AH6814; FIG. 43C).

### 4.9 Functional Characterization of PD-1-Targeted IL-7 Mimetics

To assess the cis activity of twelve IL-7R agonists targeted to PD-1, the phosphorylation of STAT5 on pre-activated CD4 T cells after 60 min incubation with test compounds was determined as previously described. The IL-2 mimetic heavy chain antibody (P1AH1177), a PD-1 dependent biparatopic IL-2 mimetic (P1AH6814+P1AI1593) and PD1-IL2v (P1AE4422) were used as reference molecules. A non-binding DP47 antibody (P1AD3966, SEQ ID NO: 230 and SEQ ID NO: 231) was used as negative control. All PD-1-targeted IL-7R agonists showed dose-dependent induction of STAT-5 phosphorylation with single-digit nM or sub-nM EC50 activity (FIG. 44).

### Example 5. Functional Characterization of Monoparatopic and Biparatopic Cis-Targeted Cytokine Receptor Agonists

To understand the requirement for biparatopic assembly of cytokine receptor agonists, we tested the monoparatopic concept of IL-2R signaling via binding to the same PD-1 epitope (FIG. 45). The previously described HEK Blue IL-2 reporter cell assay was used to assess IL-2 signaling. As previously demonstrated, biparatopic assembly of PD-1-targeted IL-2R agonists (P1AH6814+P1AI1593 and P1AH6814+P1AK2802) showed no signaling in PD-1-negative cells (FIG. 46A) and strong dose-response IL-2R signaling in PD-1-positive cells (FIG. 46B). Whereas no activity was observed with any of the eight monoparatopic pairs tested, neither in PD-1-negative nor in PD-1-positive cells (FIGS. 46A and B), highlighting the biparatopic assembly requirement for functional cytokine receptor signalling.

Sequences

| | SEQ ID | |
|---|---|---|
| Description | NO | Sequence |
| IFNγR1_1 VHH | 1 | QVQLQESGGGLVQAGGSLRLSCVASGSLFRWTA |
| | | MAWYRQAPGKQRELVAVISPAGSIDYADSVKGR |
| | | FTISRDNGKNVVYLQMNSLKPEDTAVYYCSIHHS |
| | | ARAYWGQGTQVTVSS |
| IFNγR1_2 VHH | 2 | QVQLQESGGGLVQPGGSLTLSCAASIHLFSRTAM |
| | | DWYRQAPGRQRELVATISPVGSTYYADSAKDRFT |
| | | ISRDNANNMVYLQMNSLGPEDTAVYYCISGQLQ |
| | | GQGTQVTVSS |
| IFNγR1_3 VHH | 3 | QVQLQQSGGGLVQAGGSLRLSCTASGSTFSFSNY |
| | | HMGWYRQAPGKQRERVASISSRDSTYYSDSVKG |
| | | RFTISRDTARNTVYLQMNSLEPEETAVYYCNARG |
| | | RATGRDYWGQGTQVTVSS |
| IFNγR1_4 VHH | 4 | QVQLQQFGGGLVQAGGSLRLSCTASGSTFSFSNY |
| | | HMGWYRQAPGKQRERVASISSRGSTYYSDSVKG |
| | | RFAISRDTARNTVYLQMNSLEPEETAVYYCNARG |
| | | RATGRDYWGQGTLVTVSS |
| IFNγR1_5 VHH | 5 | QVQLQQFGGGLVQPGGSLRLSCAVSRSIFSIDAM |
| | | AWYRQAPGKQRNLVGTITSDGTTNYVDSAKGRF |
| | | TISRDNAKNTVYLQMNSLKPEDTAVYFCNAGAV |
| | | SRTGGYRPSGYWGQGTLVTVSS |
| IFNγR2_1 VHH | 6 | QVQLQESGGGLVQAGGSLRLSCTASGAYFIMGW |
| | | FRQAPGKQRELVARTIRDGTTDYADSVKGRFTIS |
| | | RNTAENTAYLQMNSLKPEDTGVYYCAAGPLSKS |
| | | FAPWDPAYWGQGTLVTVSS |
| IFNγR2_2 VHH | 7 | QVQLQEFGGGLVQAGEALRLSCVASKTIFNTMP |
| | | MGWYRQAPGKERELVATITSSGVVNSADSVKGR |
| | | FTISRDSAKRTAYLQMNNLKPEDTAVYYCAAMF |
| | | KPGIPEYWGRGTQVTVSS |
| IFNγR2_3 VHH | 8 | QVQLQQFGGGLVQPGGSLTLSCAASGSIFSINPMG |
| | | WYRQAPGKRRELVASLTNRGITTYADSVKGRFTI |
| | | SRDNAKNTVYLQMNALKPEDTAVYYCHAYVDT |
| | | GSHEYPQYENYWGQGTLVTVSS |
| IFNγR2 4 VHH | 9 | QVQLQESGGGLVQAGGSLRLSCAASGSTFTFSTM |
| | | AWYRQAPGKQRELVATFFSPSTWYADSVEGRFT |
| | | VSRNNAKNTMYLHMNSLKPEDTAVYYCTSPSAS |
| | | NGQGTQVTVSS |
| IFNγR2_5 VHH | 10 | QVQLQESGGGLVQPGGSLTLSCAASGSIVSINPMG |
| | | WYRQAPGKRRELVASLTNRGITTYADSVKGRFTI |
| | | SRDNAKNTVYLQMNTLKPEDTAVYYCHAYVDT |
| | | GNYEYPQYENYWGQGTLVTVSS |
| IFNγR1_1 heavy chain | 11 | QVQLQESGGGLVQAGGSLRLSCVASGSLFRWTA |
| antibody (knob chain) | | MAWYRQAPGKQRELVAVISPAGSIDYADSVKGR |
| | | FTISRDNGKNVVYLQMNSLKPEDTAVYYCSIHHS |
| | | ARAYWGQGTQVTVSSTSGSGGGGSGGGGSGGG |
| | | GSGGGGSGGGGDKTHTCPPCPAPEAAGGPSVFLF |
| | | PPKPKDTLMISRTPEVTCVVVDVSHEDPEVKFNW |
| | | YVDGVEVHNAKTKPREEQYNSTYRVVSVLTVLH |
| | | QDWLNGKEYKCKVSNKALGAPIEKTISKAKGQP |
| | | REPQVYTLPPCRDELTKNQVSLWCLVKGFYPSDI |
| | | AVEWESNGQPENNYKTTPPVLDSDGSFFLYSKLT |
| | | VDKSRWQQGNVFSCSVMHEALHNHYTQKSLSLS |
| | | PG |
| IFNγR1_2 heavy chain | 12 | QVQLQESGGGLVQPGGSLTLSCAASIHLFSRTAM |
| antibody (knob chain) | | DWYRQAPGRQRELVATISPVGSTYYADSAKDRFT |
| | | ISRDNANNMVYLQMNSLGPEDTAVYYCISGQLQ |
| | | GQGTQVTVSSTSGSGGGGSGGGGSGGGGSGGGG |
| | | SGGGGDKTHTCPPCPAPEAAGGPSVFLFPPKPKDT |
| | | LMISRTPEVTCVVVDVSHEDPEVKFNWYVDGVE |
| | | VHNAKTKPREEQYNSTYRVVSVLTVLHQDWLNG |
| | | KEYKCKVSNKALGAPIEKTISKAKGQPREPQVYT |
| | | LPPCRDELTKNQVSLWCLVKGFYPSDIAVEWESN |
| | | GQPENNYKTTPPVLDSDGSFFLYSKLTVDKSRWQ |
| | | QGNVFSCSVMHEALHNHYTQKSLSLSPG |
| IFNγR1_3 heavy chain | 13 | QVQLQQSGGGLVQAGGSLRLSCTASGSTFSFSNY |
| antibody (knob chain) | | HMGWYRQAPGKQRERVASISSRDSTYYSDSVKG |
| | | RFTISRDTARNTVYLQMNSLEPEETAVYYCNARG |
| | | RATGRDYWGQGTQVTVSSTSGSGGGGSGGGGSG |
| | | GGGSGGGGSGGGGDKTHTCPPCPAPEAAGGPSVF |
| | | LFPPKPKDTLMISRTPEVTCVVVDVSHEDPEVKFN |
| | | WYVDGVEVHNAKTKPREEQYNSTYRVVSVLTVL |
| | | HQDWLNGKEYKCKVSNKALGAPIEKTISKAKGQ |
| | | PREPQVYTLPPCRDELTKNQVSLWCLVKGFYPSD |
| | | IAVEWESNGQPENNYKTTPPVLDSDGSFFLYSKLT |
| | | VDKSRWQQGNVFSCSVMHEALHNHYTQKSLSLS |
| | | PG |
| IFNγR1_5 heavy chain | 14 | QVQLQQFGGGLVQPGGSLRLSCAVSRSIFSIDAM |
| antibody (knob chain) | | AWYRQAPGKQRNLVGTITSDGTTNYVDSAKGRF |
| | | TISRDNAKNTVYLQMNSLKPEDTAVYFCNAGAV |
| | | SRTGGYRPSGYWGQGTLVTVSSTSGSGGGGSGG |
| | | GGSGGGGGDKTHTCPPCPAPEAAGGPSVFLFPPK |
| | | PKDTLMISRTPEVTCVVVDVSHEDPEVKFNWYV |
| | | DGVEVHNAKTKPREEQYNSTYRVVSVLTVLHQD |
| | | WLNGKEYKCKVSNKALGAPIEKTISKAKGQPREP |
| | | QVYTLPPCRDELTKNQVSLWCLVKGFYPSDIAVE |
| | | WESNGQPENNYKTTPPVLDSDGSFFLYSKLTVDK |
| | | SRWQQGNVFSCSVMHEALHNHYTQKSLSLSPG |
| IFNγR2_1 heavy chain | 15 | QVQLQESGGGLVQAGGSLRLSCTASGAYFIMGW |
| antibody (hole chain) | | FRQAPGKQRELVARTIRDGTTDYADSVKGRFTIS |
| | | RNTAENTAYLQMNSLKPEDTGVYYCAAGPLSKS |
| | | FAPWDPAYWGQGTLVTVSSTSGSGGGGSGGGGS |
| | | GGGGSGGGGSGGGGDKTHTCPPCPAPEAAGGPS |
| | | VFLFPPKPKDTLMISRTPEVTCVVVDVSHEDPEVK |
| | | FNWYVDGVEVHNAKTKPREEQYNSTYRVVSVLT |
| | | VLHQDWLNGKEYKCKVSNKALGAPIEKTISKAK |
| | | GQPREPQVCTLPPSRDELTKNQVSLSCAVKGFYPS |
| | | DIAVEWESNGQPENNYKTTPPVLDSDGSFFLVSK |
| | | LTVDKSRWQQGNVFSCSVMHEALHNHYTQKSLS |
| | | LSPG |
| IFNγR2_2 heavy chain | 16 | QVQLQEFGGGLVQAGEALRLSCVASKTIFNTMP |
| antibody (hole chain) | | MGWYRQAPGKERELVATITSSGVVNSADSVKGR |
| | | FTISRDSAKRTAYLQMNNLKPEDTAVYYCAAMF |
| | | KPGIPEYWGRGTQVTVSSTSGSGGGGGGGGSGG |
| | | GGSGGGGSGGGGDKTHTCPPCPAPEAAGGPSVFL |
| | | FPPKPKDTLMISRTPEVTCVVVDVSHEDPEVKFN |
| | | WYVDGVEVHNAKTKPREEQYNSTYRVVSVLTVL |
| | | HQDWLNGKEYKCKVSNKALGAPIEKTISKAKGQ |
| | | PREPQVCTLPPSRDELTKNQVSLSCAVKGFYPSDI |
| | | AVEWESNGQPENNYKTTPPVLDSDGSFFLVSKLT |
| | | VDKSRWQQGNVFSCSVMHEALHNHYTQKSLSLS |
| | | PG |
| IFNγR2_3 heavy chain | 17 | QVQLQQFGGGLVQPGGSLTLSCAASGSIFSINPMG |
| antibody (hole chain) | | WYRQAPGKRRELVASLTNRGITTYADSVKGRFTI |
| | | SRDNAKNTVYLQMNALKPEDTAVYYCHAYVDT |
| | | GSHEYPQYENYWGQGTLVTVSSTSGSGGGGSGG |
| | | GGSGGGGSGGGGSGGGGDKTHTCPPCPAPEAAG |
| | | GPSVFLFPPKPKDTLMISRTPEVTCVVVDVSHEDP |
| | | EVKFNWYVDGVEVHNAKTKPREEQYNSTYRVVS |
| | | VLTVLHQDWLNGKEYKCKVSNKALGAPIEKTISK |
| | | AKGQPREPQVCTLPPSRDELTKNQVSLSCAVKGF |
| | | YPSDIAVEWESNGQPENNYKTTPPVLDSDGSFFL |
| | | VSKLTVDKSRWQQGNVFSCSVMHEALHNHYTQ |
| | | KSLSLSPG |
| IFNγR2_4 heavy chain | 18 | QVQLQESGGGLVQAGGSLRLSCAASGSTFTFSTM |
| antibody (hole chain) | | AWYRQAPGKQRELVATFFSPSTWYADSVEGRFT |
| | | VSRNNAKNTMYLHMNSLKPEDTAVYYCTSPSAS |
| | | NGQGTQVTVSSTSGSGGGGSGGGGSGGGGSGGG |
| | | GSGGGGDKTHTCPPCPAPEAAGGPSVFLFPPKPK |
| | | DTLMISRTPEVTCVVVDVSHEDPEVKFNWYVDG |
| | | VEVHNAKTKPREEQYNSTYRVVSVLTVLHQDWL |
| | | NGKEYKCKVSNKALGAPIEKTISKAKGQPREPQV |
| | | CTLPPSRDELTKNQVSLSCAVKGFYPSDIAVEWES |
| | | NGQPENNYKTTPPVLDSDGSFFLVSKLTVDKSRW |
| | | QQGNVFSCSVMHEALHNHYTQKSLSLSPG |
| IFNγR2_5 heavy chain | 19 | QVQLQESGGGLVQPGGSLTLSCAASGSIVSINPMG |
| antibody (hole chain) | | WYRQAPGKRRELVASLTNRGITTYADSVKGRFTI |
| | | SRDNAKNTVYLQMNTLKPEDTAVYYCHAYVDT |
| | | GNYEYPQYENYWGQGTLVTVSSTSGSGGGGSGG |
| | | GGSGGGGSGGGGSGGGGDKTHTCPPCPAPEAAG |
| | | GPSVFLFPPKPKDTLMISRTPEVTCVVVDVSHEDP |
| | | EVKFNWYVDGVEVHNAKTKPREEQYNSTYRVVS |
| | | VLTVLHQDWLNGKEYKCKVSNKALGAPIEKTISK |
| | | AKGQPREPQVCTLPPSRDELTKNQVSLSCAVKGF |
| | | YPSDIAVEWESNGQPENNYKTTPPVLDSDGSFFL |
| | | VSKLTVDKSRWQQGNVFSCSVMHEALHNHYTQ |
| | | KSLSLSPG |
| FAP 4B9 VH | 20 | EVQLLESGGGLVQPGGSLRLSCAASGFTFSSYAM |
| | | SWVRQAPGKGLEWVSAIIGSGASTYYADSVKGRF |
| | | TISRDNSKNTLYLQMNSLRAEDTAVYYCAKGWF |
| | | GGFNYWGQGTLVTVSS |
| FAP 4B9 VL | 21 | EIVLTQSPGTLSLSPGERATLSCRASQSVTSSYLA |
| | | WYQQKPGQAPRLLINVGSRRATGIPDRFSGSGSG |
| | | TDFTLTISRLEPEDFAVYYCQQGIMLPPTFGQGTK |
| | | VEIK |
| FAP clone 1G1a | 22 | QVQLVQSGAEVKKPGASVKVSCKASGYTLTDYN |
| (humanized 212) VH | | MDWVRQAPGQGLEWIGDIYPNTGGTIYNQKFKG |
| | | RVTMTIDTSTSTVYMELSSLRSEDTAVYYCTRFR |
| | | GIHYAMDYWGQGTTVTVSS |
| FAP clone 1G1a | 23 | EIVLTQSPATLSLSPGERATLSCRASESVDNYGLSF |
| (humanized 212) VL | | INWFQQKPGQAPRLLIYGTSNRGSGIPARFSGSGS |
| | | GTDFTLTISSLEPEDFAVYFCQQSNEVPYTFGGGT |
| | | KVEIK |
| Reference molecule FAP | 24 | EVQLLESGGGLVQPGGSLRLSCAASGFTFSSYAM |
| 4B9 Fab - Fc PG LALA - | | SWVRQAPGKGLEWVSAIIGSGASTYYADSVKGRF |
| human IFNγ 1-132 | | TISRDNSKNTLYLQMNSLRAEDTAVYYCAKGWF |
| S132P (P1AF3574) HC | | GGFNYWGQGTLVTVSSASTKGPSVFPLAPSSKST |
| | | SGGTAALGCLVKDYFPEPVTVSWNSGALTSGVH |
| | | TFPAVLQSSGLYSLSSVVTVPSSSLGTQTYICNVN |
| | | HKPSNTKVDKKVEPKSCDKTHTCPPCPAPEAAGG |
| | | PSVFLFPPKPKDTLMISRTPEVTCVVVDVSHEDPE |
| | | VKFNWYVDGVEVHNAKTKPREEQYNSTYRVVS |
| | | VLTVLHQDWLNGKEYKCKVSNKALGAPIEKTISK |
| | | AKGQPREPQVYTLPPSRDELTKNQVSLTCLVKGF |
| | | YPSDIAVEWESNGQPENNYKTTPPVLDSDGSFFL |
| | | YSKLTVDKSRWQQGNVFSCSVMHEALHNHYTQ |
| | | KSLSLSPGGGGGSGGGGSGGGGSQDPYVKEAEN |
| | | LKKYFNAGHSDVADNGTLFLGILKNWKEESDRKI |
| | | MQSQIVSFYFKLFKNFKDDQSIQKSVETIKEDMN |
| | | VKFFNSNKKKRDDFEKLTNYSVTDLNVQRKAIHE |
| | | LIQVMAELSPAAKTGKRKRP |
| FAP 4B9 Fab LC | 25 | EIVLTQSPGTLSLSPGERATLSCRASQSVTSSYLA |
| (P1AF3574, P1AI0160, | | WYQQKPGQAPRLLINVGSRRATGIPDRFSGSGSG |
| P1AI4711, P1AI0183, | | TDFTLTISRLEPEDFAVYYCQQGIMLPPTFGQGTK |
| P1AI5305, P1AJ5096, | | VEIKRTVAAPSVFIFPPSDEQLKSGTASVVCLLNN |
| P1AJ5097, P1AA5355, | | FYPREAKVQWKVDNALQSGNSQESVTEQDSKDS |
| P1AJ8560, P1AJ8562, | | TYSLSSTLTLSKADYEKHKVYACEVTHQGLSSPV |
| P1AJ8564, P1AJ8566, | | TKSFNRGEC |
| P1AJ8568, P1AJ8570, | | |
| P1AJ8572, P1AJ8574, | | |
| P1AJ8576, P1AJ8578, | | |
| P1AJ8580, P1AJ8582) | | |
| Recombinant human | 26 | MQDPYVKEAENLKKYFNAGHSDVADNGTLFLGI |
| IFNγ (Peprotech) | | LKNWKEESDRKIMQSQIVSFYFKLFKNFKDDQSI |
| | | QKSVETIKEDMNVKFFNSNKKKRDDFEKLTNYSV |
| | | TDLNVQRKAIHELIQVMAELSPAAKTGKRKRSQ |
| | | MLFQGRRASQ |
| P1AI0160 knob chain | 27 | QVQLQQSGGGLVQAGGSLRLSCTASGSTFSFSNY |
| | | HMGWYRQAPGKQRERVASISSRDSTYYSDSVKG |
| | | RFTISRDTARNTVYLQMNSLEPEETAVYYCNARG |
| | | RATGRDYWGQGTQVTVSSGGSGGGGSGGGGSG |
| | | GGGSGGGGSGGEVQLLESGGGLVQPGGSLRLSC |
| | | AASGFTFSSYAMSWVRQAPGKGLEWVSAIIGSGA |
| | | STYYADSVKGRFTISRDNSKNTLYLQMNSLRAED |
| | | TAVYYCAKGWFGGFNYWGQGTLVTVSSASTKG |
| | | PSVFPLAPSSKSTSGGTAALGCLVKDYFPEPVTVS |
| | | WNSGALTSGVHTFPAVLQSSGLYSLSSVVTVPSSS |
| | | LGTQTYICNVNHKPSNTKVDKKVEPKSCDKTHTC |
| | | PPCPAPEAAGGPSVFLFPPKPKDTLMISRTPEVTC |
| | | VVVDVSHEDPEVKFNWYVDGVEVHNAKTKPRE |
| | | EQYNSTYRVVSVLTVLHQDWLNGKEYKCKVSN |
| | | KALGAPIEKTISKAKGQPREPQVYTLPPCRDELTK |
| | | NQVSLWCLVKGFYPSDIAVEWESNGQPENNYKT |
| | | TPPVLDSDGSFFLYSKLTVDKSRWQQGNVFSCSV |
| | | MHEALHNHYTQKSLSLSPG |
| Fc hole chain | 28 | DKTHTCPPCPAPEAAGGPSVFLFPPKPKDTLMISR |
| (P1AI0160, P1AI0066, | | TPEVTCVVVDVSHEDPEVKFNWYVDGVEVHNA |
| P1AI0831, P1AI0832, | | KTKPREEQYNSTYRVVSVLTVLHQDWLNGKEYK |
| P1AI4708, P1AI4709, | | CKVSNKALGAPIEKTISKAKGQPREPQVCTLPPSR |
| P1AI4710, P1AI4711, | | DELTKNQVSLSCAVKGFYPSDIAVEWESNGQPEN |
| P1AI0187, P1AI5306, | | NYKTTPPVLDSDGSFFLVSKLTVDKSRWQQGNVF |
| P1AH6850, P1AH6814, | | SCSVMHEALHNHYTQKSLSLSPG |
| P1AK2802, P1AK2803, | | |
| P1AK2809, P1AK2810, | | |
| P1AK2599, P1AK2799, | | |
| P1AK2806, P1AK2798, | | |
| P1AK3046, P1AK3048, | | |
| P1AK3051 - P1AK3054, | | |
| P1AK3063, P1AI0063, | | |
| P1AL9238-P1AL9241, | | |
| P1AL9310, P1AL9311) | | |
| LC (P1AI0066, | 29 | EIVLTQSPATLSLSPGERATLSCRASESVDNYGLSF |
| P1AI4710, P1AI0184, | | INWFQQKPGQAPRLLIYGTSNRGSGIPARFSGSGS |
| P1AI5304, P1AJ5098, | | GTDFTLTISSLEPEDFAVYFCQQSNEVPYTFGGGT |
| P1AJ5099, P1AJ8561, | | KVEIKRTVAAPSVFIFPPSDEQLKSGTASVVCLLN |
| P1AJ8563, P1AJ8565, | | NFYPREAKVQWKVDNALQSGNSQESVTEQDSKD |
| P1AJ8567, P1AJ8569, | | STYSLSSTLTLSKADYEKHKVYACEVTHQGLSSP |
| P1AJ8571, P1AJ8573, | | VTKSFNRGEC |
| P1AJ8575, P1AJ8577, | | |
| P1AJ8579, P1AJ8581, | | |
| P1AJ8583) | | |
| P1AI0066 knob chain | 30 | QVQLQEFGGGLVQAGEALRLSCVASKTIFNTMP |
| | | MGWYRQAPGKERELVATITSSGVVNSADSVKGR |
| | | FTISRDSAKRTAYLQMNNLKPEDTAVYYCAAMF |
| | | KPGIPEYWGRGTQVTVSSGGSGGGGSGGGGSGG |
| | | GGSGGGGSGGQVQLVQSGAEVKKPGASVKVSCK |
| | | ASGYTLTDYNMDWVRQAPGQGLEWIGDIYPNTG |
| | | GTIYNQKFKGRVTMTIDTSTSTVYMELSSLRSEDT |
| | | AVYYCTRFRGIHYAMDYWGQGTTVTVSSASTKG |
| | | PSVFPLAPSSKSTSGGTAALGCLVKDYFPEPVTVS |
| | | WNSGALTSGVHTFPAVLQSSGLYSLSSVVTVPSSS |
| | | LGTQTYICNVNHKPSNTKVDKKVEPKSCDKTHTC |
| | | PPCPAPEAAGGPSVFLFPPKPKDTLMISRTPEVTC |
| | | VVVDVSHEDPEVKFNWYVDGVEVHNAKTKPRE |
| | | EQYNSTYRVVSVLTVLHQDWLNGKEYKCKVSN |
| | | KALGAPIEKTISKAKGQPREPQVYTLPPCRDELTK |
| | | NQVSLWCLVKGFYPSDIAVEWESNGQPENNYKT |
| | | TPPVLDSDGSFFLYSKLTVDKSRWQQGNVFSCSV |
| | | MHEALHNHYTQKSLSLSPG |
| P1AI0831 LC | 31 | QVQLQQSGGGLVQAGGSLRLSCTASGSTFSFSNY |
| | | HMGWYRQAPGKQRERVASISSRDSTYYSDSVKG |
| | | RFTISRDTARNTVYLQMNSLEPEETAVYYCNARG |
| | | RATGRDYWGQGTQVTVSSGGSGGGGSGGGGSG |
| | | GGGSGGGGSGGEIVLTQSPGTLSLSPGERATLSCR |
| | | ASQSVTSSYLAWYQQKPGQAPRLLINVGSRRATG |
| | | IPDRFSGSGSGTDFTLTISRLEPEDFAVYYCQQGIM |
| | | LPPTFGQGTKVEIKRTVAAPSVFIFPPSDEQLKSGT |
| | | ASVVCLLNNFYPREAKVQWKVDNALQSGNSQES |
| | | VTEQDSKDSTYSLSSTLTLSKADYEKHKVYACEV |
| | | THQGLSSPVTKSFNRGEC |
| knob chain (P1AI0831, | 32 | EVQLLESGGGLVQPGGSLRLSCAASGFTFSSYAM |
| P1AI4708, P1AI0183, | | SWVRQAPGKGLEWVSAIIGSGASTYYADSVKGRF |
| P1AI5305, P1AJ5092, | | TISRDNSKNTLYLQMNSLRAEDTAVYYCAKGWF |
| P1AJ5093, P1AJ8560, | | GGFNYWGQGTLVTVSSASTKGPSVFPLAPSSKST |
| P1AJ8562, P1AJ8564, | | SGGTAALGCLVKDYFPEPVTVSWNSGALTSGVH |
| P1AJ8566, P1AJ8568, | | TFPAVLQSSGLYSLSSVVTVPSSSLGTQTYICNVN |
| P1AJ8570) | | HKPSNTKVDKKVEPKSCDKTHTCPPCPAPEAAGG |
| | | PSVFLFPPKPKDTLMISRTPEVTCVVVDVSHEDPE |
| | | VKFNWYVDGVEVHNAKTKPREEQYNSTYRVVS |
| | | VLTVLHQDWLNGKEYKCKVSNKALGAPIEKTISK |
| | | AKGQPREPQVYTLPPCRDELTKNQVSLWCLVKG |
| | | FYPSDIAVEWESNGQPENNYKTTPPVLDSDGSFFL |
| | | YSKLTVDKSRWQQGNVFSCSVMHEALHNHYTQ |
| | | KSLSLSPG |
| P1AI0832 LC | 33 | QVQLQEFGGGLVQAGEALRLSCVASKTIFNTMP |
| | | MGWYRQAPGKERELVATITSSGVVNSADSVKGR |
| | | FTISRDSAKRTAYLQMNNLKPEDTAVYYCAAMF |
| | | KPGIPEYWGRGTQVTVSSGGSGGGGSGGGGSGG |
| | | GGSGGGGSGGEIVLTQSPATLSLSPGERATLSCRA |
| | | SESVDNYGLSFINWFQQKPGQAPRLLIYGTSNRGS |
| | | GIPARFSGSGSGTDFTLTISSLEPEDFAVYFCQQSN |
| | | EVPYTFGGGTKVEIKRTVAAPSVFIFPPSDEQLKS |
| | | GTASVVCLLNNFYPREAKVQWKVDNALQSGNSQ |
| | | ESVTEQDSKDSTYSLSSTLTLSKADYEKHKVYAC |
| | | EVTHQGLSSPVTKSFNRGEC |
| knob chain (P1AI0832, | 34 | QVQLVQSGAEVKKPGASVKVSCKASGYTLTDYN |
| P1AI4709, P1AI0184, | | MDWVRQAPGQGLEWIGDIYPNTGGTIYNQKFKG |
| P1AI5304, P1AJ5094, | | RVTMTIDTSTSTVYMELSSLRSEDTAVYYCTRFR |
| P1AJ5095, P1AJ8561, | | GIHYAMDYWGQGTTVTVSSASTKGPSVFPLAPSS |
| P1AJ8563, P1AJ8565, | | KSTSGGTAALGCLVKDYFPEPVTVSWNSGALTSG |
| P1AJ8567, P1AJ8569, | | VHTFPAVLQSSGLYSLSSVVTVPSSSLGTQTYICN |
| P1AJ8571) | | VNHKPSNTKVDKKVEPKSCDKTHTCPPCPAPEAA |
| | | GGPSVFLFPPKPKDTLMISRTPEVTCVVVDVSHED |
| | | PEVKFNWYVDGVEVHNAKTKPREEQYNSTYRVV |
| | | SVLTVLHQDWLNGKEYKCKVSNKALGAPIEKTIS |
| | | KAKGQPREPQVYTLPPCRDELTKNQVSLWCLVK |
| | | GFYPSDIAVEWESNGQPENNYKTTPPVLDSDGSFF |
| | | LYSKLTVDKSRWQQGNVFSCSVMHEALHNHYTQ |
| | | KSLSLSPG |
| P1AI4708 LC | 35 | QVQLQEFGGGLVQAGEALRLSCVASKTIFNTMP |
| | | MGWYRQAPGKERELVATITSSGVVNSADSVKGR |
| | | FTISRDSAKRTAYLQMNNLKPEDTAVYYCAAMF |
| | | KPGIPEYWGRGTQVTVSSGGSGGGGSGGGGSGG |
| | | GGSGGGGSGGEIVLTQSPGTLSLSPGERATLSCRA |
| | | SQSVTSSYLAWYQQKPGQAPRLLINVGSRRATGI |
| | | PDRFSGSGSGTDFTLTISRLEPEDFAVYYCQQGIM |
| | | LPPTFGQGTKVEIKRTVAAPSVFIFPPSDEQLKSGT |
| | | ASVVCLLNNFYPREAKVQWKVDNALQSGNSQES |
| | | VTEQDSKDSTYSLSSTLTLSKADYEKHKVYACEV |
| | | THQGLSSPVTKSFNRGEC |
| P1AI4709 LC | 36 | QVQLQQSGGGLVQAGGSLRLSCTASGSTFSFSNY |
| | | HMGWYRQAPGKQRERVASISSRDSTYYSDSVKG |
| | | RFTISRDTARNTVYLQMNSLEPEETAVYYCNARG |
| | | RATGRDYWGQGTQVTVSSGGSGGGGSGGGGSG |
| | | GGGSGGGGSGGEIVLTQSPATLSLSPGERATLSCR |
| | | ASESVDNYGLSFINWFQQKPGQAPRLLIYGTSNR |
| | | GSGIPARFSGSGSGTDFTLTISSLEPEDFAVYFCQQ |
| | | SNEVPYTFGGGTKVEIKRTVAAPSVFIFPPSDEQL |
| | | KSGTASVVCLLNNFYPREAKVQWKVDNALQSGN |
| | | SQESVTEQDSKDSTYSLSSTLTLSKADYEKHKVY |
| | | ACEVTHQGLSSPVTKSFNRGEC |
| P1AI4710 knob chain | 37 | QVQLQQSGGGLVQAGGSLRLSCTASGSTFSFSNY |
| | | HMGWYRQAPGKQRERVASISSRDSTYYSDSVKG |
| | | RFTISRDTARNTVYLQMNSLEPEETAVYYCNARG |
| | | RATGRDYWGQGTQVTVSSGGSGGGGSGGGGSG |
| | | GGGSGGGGSGGQVQLVQSGAEVKKPGASVKVSC |
| | | KASGYTLTDYNMDWVRQAPGQGLEWIGDIYPNT |
| | | GGTIYNQKFKGRVTMTIDTSTSTVYMELSSLRSED |
| | | TAVYYCTRFRGIHYAMDYWGQGTTVTVSSASTK |
| | | GPSVFPLAPSSKSTSGGTAALGCLVKDYFPEPVTV |
| | | SWNSGALTSGVHTFPAVLQSSGLYSLSSVVTVPSS |
| | | SLGTQTYICNVNHKPSNTKVDKKVEPKSCDKTHT |
| | | CPPCPAPEAAGGPSVFLFPPKPKDTLMISRTPEVTC |
| | | VVVDVSHEDPEVKFNWYVDGVEVHNAKTKPRE |
| | | EQYNSTYRVVSVLTVLHQDWLNGKEYKCKVSN |
| | | KALGAPIEKTISKAKGQPREPQVYTLPPCRDELTK |
| | | NQVSLWCLVKGFYPSDIAVEWESNGQPENNYKT |
| | | TPPVLDSDGSFFLYSKLTVDKSRWQQGNVFSCSV |
| | | MHEALHNHYTQKSLSLSPG |
| P1AI4711 knob chain | 38 | QVQLQEFGGGLVQAGEALRLSCVASKTIFNTMP |
| | | MGWYRQAPGKERELVATITSSGVVNSADSVKGR |
| | | FTISRDSAKRTAYLQMNNLKPEDTAVYYCAAMF |
| | | KPGIPEYWGRGTQVTVSSGGSGGGGSGGGGSGG |
| | | GGSGGGGSGGEVQLLESGGGLVQPGGSLRLSCA |
| | | ASGFTFSSYAMSWVRQAPGKGLEWVSAIIGSGAS |
| | | TYYADSVKGRFTISRDNSKNTLYLQMNSLRAEDT |
| | | AVYYCAKGWFGGFNYWGQGTLVTVSSASTKGPS |
| | | VFPLAPSSKSTSGGTAALGCLVKDYFPEPVTVSW |
| | | NSGALTSGVHTFPAVLQSSGLYSLSSVVTVPSSSL |
| | | GTQTYICNVNHKPSNTKVDKKVEPKSCDKTHTCP |
| | | PCPAPEAAGGPSVFLFPPKPKDTLMISRTPEVTCV |
| | | VVDVSHEDPEVKFNWYVDGVEVHNAKTKPREE |
| | | QYNSTYRVVSVLTVLHQDWLNGKEYKCKVSNK |
| | | ALGAPIEKTISKAKGQPREPQVYTLPPCRDELTKN |
| | | QVSLWCLVKGFYPSDIAVEWESNGQPENNYKTTP |
| | | PVLDSDGSFFLYSKLTVDKSRWQQGNVFSCSVM |
| | | HEALHNHYTQKSLSLSPG |
| hole chain (P1AI0183, | 39 | QVQLQQSGGGLVQAGGSLRLSCTASGSTFSFSNY |
| P1AI5304) | | HMGWYRQAPGKQRERVASISSRDSTYYSDSVKG |
| | | RFTISRDTARNTVYLQMNSLEPEETAVYYCNARG |
| | | RATGRDYWGQGTQVTVSSGGSGGGGSGGGGSG |
| | | GDKTHTCPPCPAPEAAGGPSVFLFPPKPKDTLMIS |
| | | RTPEVTCVVVDVSHEDPEVKFNWYVDGVEVHNA |
| | | KTKPREEQYNSTYRVVSVLTVLHQDWLNGKEYK |
| | | CKVSNKALGAPIEKTISKAKGQPREPQVCTLPPSR |
| | | DELTKNQVSLSCAVKGFYPSDIAVEWESNGQPEN |
| | | NYKTTPPVLDSDGSFFLVSKLTVDKSRWQQGNVF |
| | | SCSVMHEALHNHYTQKSLSLSPG |
| hole chain (P1AI0184, | 40 | QVQLQEFGGGLVQAGEALRLSCVASKTIFNTMP |
| P1AI5305) | | MGWYRQAPGKERELVATITSSGVVNSADSVKGR |
| | | FTISRDSAKRTAYLQMNNLKPEDTAVYYCAAMF |
| | | KPGIPEYWGRGTQVTVSSGGSGGGGSGGGGSGG |
| | | DKTHTCPPCPAPEAAGGPSVFLFPPKPKDTLMISR |
| | | TPEVTCVVVDVSHEDPEVKFNWYVDGVEVHNA |
| | | KTKPREEQYNSTYRVVSVLTVLHQDWLNGKEYK |
| | | CKVSNKALGAPIEKTISKAKGQPREPQVCTLPPSR |
| | | DELTKNQVSLSCAVKGFYPSDIAVEWESNGQPEN |
| | | NYKTTPPVLDSDGSFFLVSKLTVDKSRWQQGNVF |
| | | SCSVMHEALHNHYTQKSLSLSPG |
| LC (P1AI0187, | 41 | EVQLLESGGGLVQPGGSLRLSCAASGFTFSSYAM |
| P1AI5306) | | SWVRQAPGKGLEWVSAIIGSGASTYYADSVKGRF |
| | | TISRDNSKNTLYLQMNSLRAEDTAVYYCAKGWF |
| | | GGFNYWGQGTLVTVSSASVAAPSVFIFPPSDEQL |
| | | KSGTASVVCLLNNFYPREAKVQWKVDNALQSGN |
| | | SQESVTEQDSKDSTYSLSSTLTLSKADYEKHKVY |
| | | ACEVTHQGLSSPVTKSFNRGEC |
| P1AI0187 knob chain | 42 | EIVLTQSPGTLSLSPGERATLSCRASQSVTSSYLA |
| | | WYQQKPGQAPRLLINVGSRRATGIPDRFSGSGSG |
| | | TDFTLTISRLEPEDFAVYYCQQGIMLPPTFGQGTK |
| | | VEIKSSASTKGPSVFPLAPSSKSTSGGTAALGCLV |
| | | KDYFPEPVTVSWNSGALTSGVHTFPAVLQSSGLY |
| | | SLSSVVTVPSSSLGTQTYICNVNHKPSNTKVDKK |
| | | VEPKSCDKTHTCPPCPAPEAAGGPSVFLFPPKPKD |
| | | TLMISRTPEVTCVVVDVSHEDPEVKFNWYVDGV |
| | | EVHNAKTKPREEQYNSTYRVVSVLTVLHQDWLN |
| | | GKEYKCKVSNKALGAPIEKTISKAKGQPREPQVY |
| | | TLPPCRDELTKNQVSLWCLVKGFYPSDIAVEWES |
| | | NGQPENNYKTTPPVLDSDGSFFLYSKLTVDKSRW |
| | | QQGNVFSCSVMHEALHNHYTQKSLSLSPGGGSG |
| | | GGGSGGGGSGGQVQLQQSGGGLVQAGGSLRLSC |
| | | TASGSTFSFSNYHMGWYRQAPGKQRERVASISSR |
| | | DSTYYSDSVKGRFTISRDTARNTVYLQMNSLEPE |
| | | ETAVYYCNARGRATGRDYWGQGTQVTVSS |
| LC (P1AI0188, | 43 | EIVLTQSPATLSLSPGERATLSCRASESVDNYGLSF |
| P1AI5307) | | INWFQQKPGQAPRLLIYGTSNRGSGIPARFSGSGS |
| | | GTDFTLTISSLEPEDFAVYFCQQSNEVPYTFGGGT |
| | | KVEIKRTVAAPSVFIFPPSDRKLKSGTASVVCLLN |
| | | NFYPREAKVQWKVDNALQSGNSQESVTEQDSKD |
| | | STYSLSSTLTLSKADYEKHKVYACEVTHQGLSSP |
| | | VTKSFNRGEC |
| knob chain (P1AI0188, | 44 | DKTHTCPPCPAPEAAGGPSVFLFPPKPKDTLMISR |
| P1AI5307, P1AH6813, | | TPEVTCVVVDVSHEDPEVKFNWYVDGVEVHNA |
| P1AI1593) | | KTKPREEQYNSTYRVVSVLTVLHQDWLNGKEYK |
| | | CKVSNKALGAPIEKTISKAKGQPREPQVYTLPPCR |
| | | DELTKNQVSLWCLVKGFYPSDIAVEWESNGQPE |
| | | NNYKTTPPVLDSDGSFFLYSKLTVDKSRWQQGN |
| | | VFSCSVMHEALHNHYTQKSLSLSPG |
| P1AI0188 hole chain | 45 | QVQLVQSGAEVKKPGASVKVSCKASGYTLTDYN |
| | | MDWVRQAPGQGLEWIGDIYPNTGGTIYNQKFKG |
| | | RVTMTIDTSTSTVYMELSSLRSEDTAVYYCTRFR |
| | | GIHYAMDYWGQGTTVTVSSASTKGPSVFPLAPSS |
| | | KSTSGGTAALGCLVEDYFPEPVTVSWNSGALTSG |
| | | VHTFPAVLQSSGLYSLSSVVTVPSSSLGTQTYICN |
| | | VNHKPSNTKVDEKVEPKSCDKTHTCPPCPAPEAA |
| | | GGPSVFLFPPKPKDTLMISRTPEVTCVVVDVSHED |
| | | PEVKFNWYVDGVEVHNAKTKPREEQYNSTYRVV |
| | | SVLTVLHQDWLNGKEYKCKVSNKALGAPIEKTIS |
| | | KAKGQPREPQVCTLPPSRDELTKNQVSLSCAVKG |
| | | FYPSDIAVEWESNGQPENNYKTTPPVLDSDGSFFL |
| | | VSKLTVDKSRWQQGNVFSCSVMHEALHNHYTQ |
| | | KSLSLSPGGGSGGGGSGGGGSGGQVQLQEFGGG |
| | | LVQAGEALRLSCVASKTIFNTMPMGWYRQAPGK |
| | | ERELVATITSSGVVNSADSVKGRFTISRDSAKRTA |
| | | YLQMNNLKPEDTAVYYCAAMFKPGIPEYWGRGT |
| | | QVTVSS |
| P1AI5306 knob chain | 46 | EIVLTQSPGTLSLSPGERATLSCRASQSVTSSYLA |
| | | WYQQKPGQAPRLLINVGSRRATGIPDRFSGSGSG |
| | | TDFTLTISRLEPEDFAVYYCQQGIMLPPTFGQGTK |
| | | VEIKSSASTKGPSVFPLAPSSKSTSGGTAALGCLV |
| | | KDYFPEPVTVSWNSGALTSGVHTFPAVLQSSGLY |
| | | SLSSVVTVPSSSLGTQTYICNVNHKPSNTKVDKK |
| | | VEPKSCDKTHTCPPCPAPEAAGGPSVFLFPPKPKD |
| | | TLMISRTPEVTCVVVDVSHEDPEVKFNWYVDGV |
| | | EVHNAKTKPREEQYNSTYRVVSVLTVLHQDWLN |
| | | GKEYKCKVSNKALGAPIEKTISKAKGQPREPQVY |
| | | TLPPCRDELTKNQVSLWCLVKGFYPSDIAVEWES |
| | | NGQPENNYKTTPPVLDSDGSFFLYSKLTVDKSRW |
| | | QQGNVFSCSVMHEALHNHYTQKSLSLSPGGGSG |
| | | GGGSGGGGSGGQVQLQEFGGGLVQAGEALRLSC |
| | | VASKTIFNTMPMGWYRQAPGKERELVATITSSGV |
| | | VNSADSVKGRFTISRDSAKRTAYLQMNNLKPEDT |
| | | AVYYCAAMFKPGIPEYWGRGTQVTVSS |
| P1AI5307 hole chain | 47 | QVQLVQSGAEVKKPGASVKVSCKASGYTLTDYN |
| | | MDWVRQAPGQGLEWIGDIYPNTGGTIYNQKFKG |
| | | RVTMTIDTSTSTVYMELSSLRSEDTAVYYCTRFR |
| | | GIHYAMDYWGQGTTVTVSSASTKGPSVFPLAPSS |
| | | KSTSGGTAALGCLVEDYFPEPVTVSWNSGALTSG |
| | | VHTFPAVLQSSGLYSLSSVVTVPSSSLGTQTYICN |
| | | VNHKPSNTKVDEKVEPKSCDKTHTCPPCPAPEAA |
| | | GGPSVFLFPPKPKDTLMISRTPEVTCVVVDVSHED |
| | | PEVKFNWYVDGVEVHNAKTKPREEQYNSTYRVV |
| | | SVLTVLHQDWLNGKEYKCKVSNKALGAPIEKTIS |
| | | KAKGQPREPQVCTLPPSRDELTKNQVSLSCAVKG |
| | | FYPSDIAVEWESNGQPENNYKTTPPVLDSDGSFFL |
| | | VSKLTVDKSRWQQGNVFSCSVMHEALHNHYTQ |
| | | KSLSLSPGGGSGGGGSGGGGSGGQVQLQQSGGG |
| | | LVQAGGSLRLSCTASGSTFSFSNYHMGWYRQAP |
| | | GKQRERVASISSRDSTYYSDSVKGRFTISRDTARN |
| | | TVYLQMNSLEPEETAVYYCNARGRATGRDYWG |
| | | QGTQVTVSS |
| IFNγR1 ECD fused to | 48 | EMGTADLGPSSVPTPTNVTIESYNMNPIVYWEYQI |
| biotinylated Fc knob | | MPQVPVFTVEVKNYGVKNSEWIDACINISHHYCN |
| (P1AG0843, P1AG1987) | | ISDHVGDPSNSLWVRVKARVGQKESAYAKSEEF |
| | | AVCRDGKIGPPKLDIRKEEKQIMIDIFHPSVFVNG |
| | | DEQEVDYDPETTCYIRVYNVYVRMNGSEIQYKIL |
| | | TQKEDDCDEIQCQLAIPVSSLNSQYCVSAEGVLH |
| | | VWGVTTEKSKEVCITIVNSSGGGGSGGGGDKTHT |
| | | CPPCPAPELLGGPSVFLFPPKPKDTLMISRTPEVTC |
| | | VVVDVSHEDPEVKFNWYVDGVEVHNAKTKPRE |
| | | EQYNSTYRVVSVLTVLHQDWLNGKEYKCKVSN |
| | | KALPAPIEKTISKAKGQPREPQVYTLPPCRDELTK |
| | | NQVSLWCLVKGFYPSDIAVEWESNGQPENNYKT |
| | | TPPVLDSDGSFFLYSKLTVDKSRWQQGNVFSCSV |
| | | MHEALHNHYTQKSLSLSPGSGGLNDIFEAQKIEW |
| | | HE |
| IFNγR2 C174S ECD | 49 | SQLPAPQHPKIRLYNAEQVLSWEPVALSNSTRPV |
| fused Twin-Strep tagged | | VYQVQFKYTDSKWFTADIMSIGVNCTQITATECD |
| Fc hole (P1AG0843, | | FTAASPSAGFPMDFNVTLRLRAELGALHSAWVT |
| P1AG6735) | | MPWFQHYRNVTVGPPENIEVTPGEGSLIIRFSSPFD |
| | | IADTSTAFFSYYVHYWEKGGIQQVKGPFRSNSISL |
| | | DNLKPSRVYCLQVQAQLLWNKSNIFRVGHLSNIS |
| | | CYETMADASTELQQGGGGSGGGGDKTHTCPPCP |
| | | APELLGGPSVFLFPPKPKDTLMISRTPEVTCVVVD |
| | | VSHEDPEVKFNWYVDGVEVHNAKTKPREEQYNS |
| | | TYRVVSVLTVLHQDWLNGKEYKCKVSNKALPAP |
| | | IEKTISKAKGQPREPQVCTLPPSRDELTKNQVSLS |
| | | CAVKGFYPSDIAVEWESNGQPENNYKTTPPVLDS |
| | | DGSFFLVSKLTVDKSRWQQGNVFSCSVMHEALH |
| | | NHYTQKSLSLSPGSGGGSAWSHPQFEKGGGSGG |
| | | GSGGSAWSHPQFEK |
| empty Fc hole | 50 | DKTHTCPPCPAPELLGGPSVFLFPPKPKDTLMISRT |
| (P1AG1987) | | PEVTCVVVDVSHEDPEVKFNWYVDGVEVHNAK |
| | | TKPREEQYNSTYRVVSVLTVLHQDWLNGKEYKC |
| | | KVSNKALPAPIEKTISKAKGQPREPQVCTLPPSRD |
| | | ELTKNQVSLSCAVKGFYPSDIAVEWESNGQPENN |
| | | YKTTPPVLDSDGSFFLVSKLTVDKSRWQQGNVFS |
| | | CSVMHEALHNHYTQKSLSLSPG |
| empty biotinylated Fc | 51 | DKTHTCPPCPAPELLGGPSVFLFPPKPKDTLMISRT |
| knob (P1AG6735, | | PEVTCVVVDVSHEDPEVKFNWYVDGVEVHNAK |
| P1AE6073, P1AF1106) | | TKPREEQYNSTYRVVSVLTVLHQDWLNGKEYKC |
| | | KVSNKALPAPIEKTISKAKGQPREPQVYTLPPCRD |
| | | ELTKNQVSLWCLVKGFYPSDIAVEWESNGQPEN |
| | | NYKTTPPVLDSDGSFFLYSKLTVDKSRWQQGNVF |
| | | SCSVMHEALHNHYTQKSLSLSPGKSGGLNDIFEA |
| | | QKIEWHE |
| Human Fc AviTag | 52 | GLNDIFEAQKIEWHEDKTHTCPPCPAPELLGGPSV |
| (P1AD4290)(knob | | FLFPPKPKDTLMISRTPEVTCVVVDVSHEDPEVKF |
| chain) | | NWYVDGVEVHNAKTKPREEQYNSTYRVVSVLT |
| | | VLHQDWLNGKEYKCKVSNKALPAPIEKTISKAKG |
| | | QPREPQVYTLPPCRDELTKNQVSLWCLVKGFYPS |
| | | DIAVEWESNGQPENNYKTTPPVLDSDGSFFLYSK |
| | | LTVDKSRWQQGNVFSCSVMHEALHNHYTQKSLS |
| | | LSPGK |
| Human Fc AviTag | 53 | DKTHTCPPCPAPELLGGPSVFLFPPKPKDTLMISRT |
| (P1AD4290)(hole chain) | | PEVTCVVVDVSHEDPEVKFNWYVDGVEVHNAK |
| | | TKPREEQYNSTYRVVSVLTVLHQDWLNGKEYKC |
| | | KVSNKALPAPIEKTISKAKGQPREPQVCTLPPSRD |
| | | ELTKNQVSLSCAVKGFYPSDIAVEWESNGQPENN |
| | | YKTTPPVLDSDGSFFLVSKLTVDKSRWQQGNVFS |
| | | CSVMHEALHNHYTQKSLSLSPGKSGGLNDIFEAQ |
| | | KIEWHE |
| Human Fc AviTag | 54 | DKTHTCPPCPAPELLGGPSVFLFPPKPKDTLMISRT |
| biotinylated (P1AE6073) | | PEVTCVVVDVSHEDPEVKFNWYVDGVEVHNAK |
| (hole chain) | | TKPREEQYNSTYRVVSVLTVLHQDWLNGKEYKC |
| | | KVSNKALPAPIEKTISKAKGQPREPQVCTLPPSRD |
| | | ELTKNQVSLSCAVKGFYPSDIAVEWESNGQPENN |
| | | YKTTPPVLDSDGSFFLVSKLTVDKSRWQQGNVFS |
| | | CSVMHEALHNRFTQKSLSLSPGK |
| IFNγR1_4 VHH Fc hole | 55 | QVQLQQFGGGLVQAGGSLRLSCTASGSTFSFSNY |
| | | HMGWYRQAPGKQRERVASISSRGSTYYSDSVKG |
| | | RFAISRDTARNTVYLQMNSLEPEETAVYYCNARG |
| | | RATGRDYWGQGTLVTVSSTSGSGGGGSGGGGSG |
| | | GGGSGGGGSGGGGDKTHTCPPCPAPEAAGGPSVF |
| | | LFPPKPKDTLMISRTPEVTCVVVDVSHEDPEVKFN |
| | | WYVDGVEVHNAKTKPREEQYNSTYRVVSVLTVL |
| | | HQDWLNGKEYKCKVSNKALGAPIEKTISKAKGQ |
| | | PREPQVYTLPPCRDELTKNQVSLWCLVKGFYPSD |
| | | IAVEWESNGQPENNYKTTPPVLDSDGSFFLYSKLT |
| | | VDKSRWQQGNVFSCSVMHEALHNHYTQKSLSLS |
| | | PG |
| IL-2Rβ_1 VHH | 56 | QVQLAESGGGLVQPGGSLTLSCVASGSILEMSRM |
| | | AWYRQAPGKMRELVASITSIGSIVYADSVKGRFT |
| | | LSRDNTKNTVYLQMNSLKPEDTAVYRCNALGTW |
| | | SDETYWGQGTQVTVSS |
| IL-2Rβ_2 VHH | 57 | QVQLQQFGGGLVQPGGSLTLSCVASESISEMSRM |
| | | AWYRQAPGKQRELVASITMHGGVVYADAVKGR |
| | | FTISRDNTKNTVYLQMNSLKPEDTAVYRCNALGT |
| | | WSDETYWGQGTLVTVSS |
| IL-2Rß_3 VHH | 58 | QVQLQESGGGLVQPGGSLTLSCVASGSILEMSRM |
| | | AWYRQAPGKQRELVASITSIGSKVYADSVKGRFT |
| | | LSRDNTKNTIYLQMNSLKPEDTAVYRCNLLGTWS |
| | | DETYWGQGTLVTVSS |
| IL-2Rβ_4 VHH | 59 | QVQLQASGGGLVQSGGSLRLSCVASGSTFSINGM |
| | | GWYRQAPGKERELVATLTAGGNADYAVSVAGR |
| | | FIISRGDKKNTRILQMNDLKPEDTAVYYCMADIY |
| | | TGDSYSGVDYWGKGTLVTVSS |
| IL-2Rβ_5 VHH | 60 | QVQLQQSGGGLVQPGGSLTLSCVASGSILEMSRM |
| | | AWYRQAPGKMRELVASITSIGSIVYADSVKGRFT |
| | | LSRDNTKNTVYLQMNSLKPEDTAVYRCNLLGTW |
| | | SDETYWGQGTQVTVSS |
| IL-2Rγ_1 VHH | 61 | QVQLQQFGGGSVQAGGSLRLSCSAPGIIFEDIVMG |
| | | WYRQGPGKQRELVALINSAVTDYADSVKGRFTIS |
| | | RDNAKNLVYLQMNSLRSEDTAVYYCTAIDINWA |
| | | QYWGQGTQVTVSS |
| IL-2Rγ_2 VHH | 62 | QVQLQESGGGLVQAGGSLRLSCAASISISKIDLMG |
| | | WYRQAPGRQRELVARVTNGGDSYYSTSVKGRFT |
| | | ISRDNAKNTLYLQMNSLKPEDTAVYYCYGVPESL |
| | | AAFHWGQGTLVTVSS |
| IL-2Rγ_3 VHH | 63 | QVQLQESGGGLVQPGGSLRLSCAASGSIFSGNAM |
| | | GWYRQAPGKQRELVASITSGGDTHYVDSVKGRF |
| | | TISRDNAKNMVYLQMSSLKPEDTAVYYCNAQER |
| | | VYSDYAFASWGPGTQVTVSS |
| IL-2Rγ_4 VHH | 64 | QVQLQESGGGLVQPGGSLRLSCAASGFTFSTYVM |
| | | NWVRQAPGKGLEWVSGIDSDGDSTTYTDSVKGR |
| | | FTISRDNAKNTLYLQMNSLKPEDTAVYYCSMFR |
| | | MATTGSQGTQVTVSS |
| IL-2Rγ_5 VHH | 65 | QVQLQESGGGLVQAGGSLRLSCAASGITSSIYAM |
| | | GWYRQAPGNEREPVALITSGDNTNYPDSVKGRFT |
| | | ISRDTAKNTVYLQMSSLKPEDTAVYYCYADVVIG |
| | | TTYYTSWGQGTLVTVSS |
| IL2Rβ_1 heavy chain | 66 | QVQLAESGGGLVQPGGSLTLSCVASGSILEMSRM |
| antibody (knob chain) | | AWYRQAPGKMRELVASITSIGSIVYADSVKGRFT |
| | | LSRDNTKNTVYLQMNSLKPEDTAVYRCNALGTW |
| | | SDETYWGQGTQVTVSSTSGSGGGGSGGGGSGGG |
| | | GSGGGGSGGGGDKTHTCPPCPAPEAAGGPSVFLF |
| | | PPKPKDTLMISRTPEVTCVVVDVSHEDPEVKFNW |
| | | YVDGVEVHNAKTKPREEQYNSTYRVVSVLTVLH |
| | | QDWLNGKEYKCKVSNKALGAPIEKTISKAKGQP |
| | | REPQVYTLPPCRDELTKNQVSLWCLVKGFYPSDI |
| | | AVEWESNGQPENNYKTTPPVLDSDGSFFLYSKLT |
| | | VDKSRWQQGNVFSCSVMHEALHNHYTQKSLSLS |
| | | PG |
| IL2Rβ_2 heavy chain | 67 | QVQLQQFGGGLVQPGGSLTLSCVASESISEMSRM |
| antibody (knob chain) | | AWYRQAPGKQRELVASITMHGGVVYADAVKGR |
| | | FTISRDNTKNTVYLQMNSLKPEDTAVYRCNALGT |
| | | WSDETYWGQGTLVTVSSTSGSGGGGSGGGGSGG |
| | | GGSGGGGSGGGGDKTHTCPPCPAPEAAGGPSVFL |
| | | FPPKPKDTLMISRTPEVTCVVVDVSHEDPEVKFN |
| | | WYVDGVEVHNAKTKPREEQYNSTYRVVSVLTVL |
| | | HQDWLNGKEYKCKVSNKALGAPIEKTISKAKGQ |
| | | PREPQVYTLPPCRDELTKNQVSLWCLVKGFYPSD |
| | | IAVEWESNGQPENNYKTTPPVLDSDGSFFLYSKLT |
| | | VDKSRWQQGNVFSCSVMHEALHNHYTQKSLSLS |
| | | PG |
| IL2Rβ_3 heavy chain | 68 | QVQLQESGGGLVQPGGSLTLSCVASGSILEMSRM |
| antibody (knob chain) | | AWYRQAPGKQRELVASITSIGSKVYADSVKGRFT |
| | | LSRDNTKNTIYLQMNSLKPEDTAVYRCNLLGTWS |
| | | DETYWGQGTLVTVSSTSGSGGGGSGGGGSGGGG |
| | | SGGGGSGGGGDKTHTCPPCPAPEAAGGPSVFLFP |
| | | PKPKDTLMISRTPEVTCVVVDVSHEDPEVKFNWY |
| | | VDGVEVHNAKTKPREEQYNSTYRVVSVLTVLHQ |
| | | DWLNGKEYKCKVSNKALGAPIEKTISKAKGQPRE |
| | | PQVYTLPPCRDELTKNQVSLWCLVKGFYPSDIAV |
| | | EWESNGQPENNYKTTPPVLDSDGSFFLYSKLTVD |
| | | KSRWQQGNVFSCSVMHEALHNHYTQKSLSLSPG |
| IL2Rβ_5 heavy chain | 69 | QVQLQQSGGGLVQPGGSLTLSCVASGSILEMSRM |
| antibody (knob chain) | | AWYRQAPGKMRELVASITSIGSIVYADSVKGRFT |
| | | LSRDNTKNTVYLQMNSLKPEDTAVYRCNLLGTW |
| | | SDETYWGQGTQVTVSSTSGSGGGGSGGGGSGGG |
| | | GSGGGGSGGGGDKTHTCPPCPAPEAAGGPSVFLF |
| | | PPKPKDTLMISRTPEVTCVVVDVSHEDPEVKFNW |
| | | YVDGVEVHNAKTKPREEQYNSTYRVVSVLTVLH |
| | | QDWLNGKEYKCKVSNKALGAPIEKTISKAKGQP |
| | | REPQVYTLPPCRDELTKNQVSLWCLVKGFYPSDI |
| | | AVEWESNGQPENNYKTTPPVLDSDGSFFLYSKLT |
| | | VDKSRWQQGNVFSCSVMHEALHNHYTQKSLSLS |
| | | PG |
| IL2Rγ_2 heavy chain | 70 | QVQLQESGGGLVQAGGSLRLSCAASISISKIDLMG |
| antibody (hole chain) | | WYRQAPGRQRELVARVTNGGDSYYSTSVKGRFT |
| | | ISRDNAKNTLYLQMNSLKPEDTAVYYCYGVPESL |
| | | AAFHWGQGTLVTVSSTSGSGGGGSGGGGSGGGG |
| | | SGGGGSGGGGDKTHTCPPCPAPEAAGGPSVFLFP |
| | | PKPKDTLMISRTPEVTCVVVDVSHEDPEVKFNWY |
| | | VDGVEVHNAKTKPREEQYNSTYRVVSVLTVLHQ |
| | | DWLNGKEYKCKVSNKALGAPIEKTISKAKGQPRE |
| | | PQVCTLPPSRDELTKNQVSLSCAVKGFYPSDIAVE |
| | | WESNGQPENNYKTTPPVLDSDGSFFLVSKLTVDK |
| | | SRWQQGNVFSCSVMHEALHNHYTQKSLSLSPG |
| IL2Rγ_3 heavy chain | 71 | QVQLQESGGGLVQPGGSLRLSCAASGSIFSGNAM |
| antibody (hole chain) | | GWYRQAPGKQRELVASITSGGDTHYVDSVKGRF |
| | | TISRDNAKNMVYLQMSSLKPEDTAVYYCNAQER |
| | | VYSDYAFASWGPGTQVTVSSTSGSGGGGSGGGG |
| | | SGGGGSGGGGSGGGGDKTHTCPPCPAPEAAGGPS |
| | | VFLFPPKPKDTLMISRTPEVTCVVVDVSHEDPEVK |
| | | FNWYVDGVEVHNAKTKPREEQYNSTYRVVSVLT |
| | | VLHQDWLNGKEYKCKVSNKALGAPIEKTISKAK |
| | | GQPREPQVCTLPPSRDELTKNQVSLSCAVKGFYPS |
| | | DIAVEWESNGQPENNYKTTPPVLDSDGSFFLVSK |
| | | LTVDKSRWQQGNVFSCSVMHEALHNHYTQKSLS |
| | | LSPG |
| IL2Rγ_4 heavy chain | 72 | QVQLQESGGGLVQPGGSLRLSCAASGFTFSTYVM |
| antibody (hole chain) | | NWVRQAPGKGLEWVSGIDSDGDSTTYTDSVKGR |
| | | FTISRDNAKNTLYLQMNSLKPEDTAVYYCSMFR |
| | | MATTGSQGTQVTVSSTSGSGGGGSGGGGSGGGG |
| | | SGGGGSGGGGDKTHTCPPCPAPEAAGGPSVFLFP |
| | | PKPKDTLMISRTPEVTCVVVDVSHEDPEVKFNWY |
| | | VDGVEVHNAKTKPREEQYNSTYRVVSVLTVLHQ |
| | | DWLNGKEYKCKVSNKALGAPIEKTISKAKGQPRE |
| | | PQVCTLPPSRDELTKNQVSLSCAVKGFYPSDIAVE |
| | | WESNGQPENNYKTTPPVLDSDGSFFLVSKLTVDK |
| | | SRWQQGNVFSCSVMHEALHNHYTQKSLSLSPG |
| IL2Rγ_5 heavy chain | 73 | QVQLQESGGGLVQAGGSLRLSCAASGITSSIYAM |
| antibody (hole chain) | | GWYRQAPGNEREPVALITSGDNTNYPDSVKGRFT |
| | | ISRDTAKNTVYLQMSSLKPEDTAVYYCYADVVIG |
| | | TTYYTSWGQGTLVTVSSTSGSGGGGSGGGGSGG |
| | | GGSGGGGSGGGGDKTHTCPPCPAPEAAGGPSVFL |
| | | FPPKPKDTLMISRTPEVTCVVVDVSHEDPEVKFN |
| | | WYVDGVEVHNAKTKPREEQYNSTYRVVSVLTVL |
| | | HQDWLNGKEYKCKVSNKALGAPIEKTISKAKGQ |
| | | PREPQVCTLPPSRDELTKNQVSLSCAVKGFYPSDI |
| | | AVEWESNGQPENNYKTTPPVLDSDGSFFLVSKLT |
| | | VDKSRWQQGNVFSCSVMHEALHNHYTQKSLSLS |
| | | PG |
| IL-2Rβ_4 VHH Fc knob | 74 | QVQLQASGGGLVQSGGSLRLSCVASGSTFSINGM |
| | | GWYRQAPGKERELVATLTAGGNADYAVSVAGR |
| | | FIISRGDKKNTRILQMNDLKPEDTAVYYCMADIY |
| | | TGDSYSGVDYWGKGTLVTVSSTSGSGGGGSGGG |
| | | GSGGGGGDKTHTCPPCPAPEAAGGPSVFLFPPKP |
| | | KDTLMISRTPEVTCVVVDVSHEDPEVKFNWYVD |
| | | GVEVHNAKTKPREEQYNSTYRVVSVLTVLHQDW |
| | | LNGKEYKCKVSNKALGAPIEKTISKAKGQPREPQ |
| | | VYTLPPCRDELTKNQVSLWCLVKGFYPSDIAVEW |
| | | ESNGQPENNYKTTPPVLDSDGSFFLYSKLTVDKS |
| | | RWQQGNVFSCSVMHEALHNHYTQKSLSLSPG |
| IL-2Rγ_1 VHH Fc hole | 75 | QVQLQQFGGGSVQAGGSLRLSCSAPGIIFEDIVMG |
| | | WYRQGPGKQRELVALINSAVTDYADSVKGRFTIS |
| | | RDNAKNLVYLQMNSLRSEDTAVYYCTAIDTNWA |
| | | QYWGQGTQVTVSSTSGSGGGGSGGGGSGGGGSG |
| | | GGGSGGGGDKTHTCPPCPAPEAAGGPSVFLFPPK |
| | | PKDTLMISRTPEVTCVVVDVSHEDPEVKFNWYV |
| | | DGVEVHNAKTKPREEQYNSTYRVVSVLTVLHQD |
| | | WLNGKEYKCKVSNKALGAPIEKTISKAKGQPREP |
| | | QVCTLPPSRDELTKNQVSLSCAVKGFYPSDIAVE |
| | | WESNGQPENNYKTTPPVLDSDGSFFLVSKLTVDK |
| | | SRWQQGNVFSCSVMHEALHNHYTQKSLSLSPG |
| PD-1 binder 1 0376 VH | 76 | EVQLLESGGGLVQPGGSLRLSCAASGFSFSSYTMS |
| | | WVRQAPGKGLEWVATISGGGRDIYYPDSVKGRF |
| | | TISRDNSKNTLYLQMNSLRAEDTAVYYCVLLTGR |
| | | VYFALDSWGQGTLVTVSS |
| PD-1 binder 1 0376 VL | 77 | DIVMTQSPDSLAVSLGERATINCKASESVDTSDNS |
| | | FIHWYQQKPGQSPKLLIYRSSTLESGVPDRFSGSG |
| | | SGTDFTLTISSLQAEDVAVYYCQQNYDVPWTFGQ |
| | | GTKVEIK |
| PD-1 binder 2 1040 VH | 78 | EVQLLESGGGLVQPGGSLRLSCAASGFTFSSYAM |
| | | SWVRQAPGKGLEWVSAITGSGGSTYYADSVKGR |
| | | FTISRDNSRNTLYLQMNSLRAEDTAVYYCAKGEG |
| | | YAGSSYFRASDIWGQGTMVTVSS |
| PD-1 binder 2 1040 VL | 79 | DIQMTQSPSSLSASVGDRVTITCRASQSISSYLNW |
| | | YQQKPGKAPKLLIYTASSLQSGVPSRFSGSGSGTD |
| | | FTLTISSLQPEDFATYYCQQSYSTPLTFGGGTKVEI |
| | | K |
| human IL-2Rβ Fc | 80 | AVNGTSQFTCFYNSRANISCVWSQDGALQDTSCQ |
| biotinylated (P1AE2657, | | VHAWPDRRRWNQTCELLPVSQASWACNLILGAP |
| P1AF1104)(knob chain) | | DSQKLTTVDIVTLRVLCREGVRWRVMAIQDFKPF |
| | | ENLRLMAPISLQVVHVETHRCNISWEISQASHYFE |
| | | RHLEFEARTLSPGHTWEEAPLLTLKQKQEWICLE |
| | | TLTPDTQYEFQVRVKPLQGEFTTWSPWSQPLAFR |
| | | TKPAALGKDTGAQDKTHTCPPCPAPELLGGPSVF |
| | | LFPPKPKDTLMISRTPEVTCVVVDVSHEDPEVKFN |
| | | WYVDGVEVHNAKTKPREEQYNSTYRVVSVLTVL |
| | | HQDWLNGKEYKCKVSNKALPAPIEKTISKAKGQP |
| | | REPQVYTLPPCRDELTKNQVSLWCLVKGFYPSDI |
| | | AVEWESNGQPENNYKTTPPVLDSDGSFFLYSKLT |
| | | VDKSRWQQGNVFSCSVMHEALHNHYTQKSLSLS |
| | | PGKSGGLNDIFEAQKIEWHE |
| human IL-2Rγ Fc | 81 | LNTTILTPNGNEDTTADFFLTTMPTDSLSVSTLPLP |
| (P1AE2657, P1AF1106) | | EVQCFVFNVEYMNCTWNSSSEPQPTNLTLHYWY |
| (hole chain) | | KNSDNDKVQKCSHYLFSEEITSGCQLQKKEIHLY |
| | | QTFVVQLQDPREPRRQATQMLKLQNLVIPWAPE |
| | | NLTLHKLSESQLELNWNNRFLNHCLEHLVQYRT |
| | | DWDHSWTEQSVDYRHKFSLPSVDGQKRYTFRVR |
| | | SRFNPLCGSAQHWSEWSHPIHWGSNTSKENPFLF |
| | | ALEAGAQDKTHTCPPCPAPELLGGPSVFLFPPKPK |
| | | DTLMISRTPEVTCVVVDVSHEDPEVKFNWYVDG |
| | | VEVHNAKTKPREEQYNSTYRVVSVLTVLHQDWL |
| | | NGKEYKCKVSNKALPAPIEKTISKAKGQPREPQV |
| | | CTLPPSRDELTKNQVSLSCAVKGFYPSDIAVEWES |
| | | NGQPENNYKTTPPVLDSDGSFFLVSKLTVDKSRW |
| | | QQGNVFSCSVMHEALHNHYTQKSLSLSPGK |
| Monovalent human IL- | 82 | DKTHTCPPCPAPELLGGPSVFLFPPKPKDTLMISRT |
| 2Rβ Fc biotinylated | | PEVTCVVVDVSHEDPEVKFNWYVDGVEVHNAK |
| (P1AF1104)(hole chain) | | TKPREEQYNSTYRVVSVLTVLHQDWLNGKEYKC |
| | | KVSNKALPAPIEKTISKAKGQPREPQVCTLPPSRD |
| | | ELTKNQVSLSCAVKGFYPSDIAVEWESNGQPENN |
| | | YKTTPPVLDSDGSFFLVSKLTVDKSRWQQGNVFS |
| | | CSVMHEALHNHYTQKSLSLSPGK |
| Anti-PD1 LC | 83 | DIVMTQSPDSLAVSLGERATINCKASESVDTSDNS |
| (P1AE4422, P1AH6813) | | FIHWYQQKPGQSPKLLIYRSSTLESGVPDRFSGSG |
| | | SGTDFTLTISSLQAEDVAVYYCQQNYDVPWTFGQ |
| | | GTKVEIKRTVAAPSVFIFPPSDEQLKSGTASVVCL |
| | | LNNFYPREAKVQWKVDNALQSGNSQESVTEQDS |
| | | KDSTYSLSSTLTLSKADYEKHKVYACEVTHQGLS |
| | | SPVTKSFNRGEC |
| Anti-PD1-IL2v | 84 | EVQLLESGGGLVQPGGSLRLSCAASGFSFSSYTMS |
| (P1AE4422) knob chain | | WVRQAPGKGLEWVATISGGGRDIYYPDSVKGRF |
| | | TISRDNSKNTLYLQMNSLRAEDTAVYYCVLLTGR |
| | | VYFALDSWGQGTLVTVSSASTKGPSVFPLAPSSK |
| | | STSGGTAALGCLVKDYFPEPVTVSWNSGALTSGV |
| | | HTFPAVLQSSGLYSLSSVVTVPSSSLGTQTYICNV |
| | | NHKPSNTKVDKKVEPKSCDKTHTCPPCPAPEAAG |
| | | GPSVFLFPPKPKDTLMISRTPEVTCVVVDVSHEDP |
| | | EVKFNWYVDGVEVHNAKTKPREEQYNSTYRVVS |
| | | VLTVLHQDWLNGKEYKCKVSNKALGAPIEKTISK |
| | | AKGQPREPQVYTLPPCRDELTKNQVSLWCLVKG |
| | | FYPSDIAVEWESNGQPENNYKTTPPVLDSDGSFFL |
| | | YSKLTVDKSRWQQGNVFSCSVMHEALHNHYTQ |
| | | KSLSLSPGGGGGSGGGGSGGGGSAPASSSTKKTQ |
| | | LQLEHLLLDLQMILNGINNYKNPKLTRMLTAKFA |
| | | MPKKATELKHLQCLEEELKPLEEVLNGAQSKNFH |
| | | LRPRDLISNINVIVLELKGSETTFMCEYADETATIV |
| | | EFLNRWITFAQSIISTLT |
| Anti-PD1-IL2v | 85 | EVQLLESGGGLVQPGGSLRLSCAASGFSFSSYTMS |
| (P1AE4422) hole chain | | WVRQAPGKGLEWVATISGGGRDIYYPDSVKGRF |
| | | TISRDNSKNTLYLQMNSLRAEDTAVYYCVLLTGR |
| | | VYFALDSWGQGTLVTVSSASTKGPSVFPLAPSSK |
| | | STSGGTAALGCLVKDYFPEPVTVSWNSGALTSGV |
| | | HTFPAVLQSSGLYSLSSVVTVPSSSLGTQTYICNV |
| | | NHKPSNTKVDKKVEPKSCDKTHTCPPCPAPEAAG |
| | | GPSVFLFPPKPKDTLMISRTPEVTCVVVDVSHEDP |
| | | EVKFNWYVDGVEVHNAKTKPREEQYNSTYRVVS |
| | | VLTVLHQDWLNGKEYKCKVSNKALGAPIEKTISK |
| | | AKGQPREPQVCTLPPSRDELTKNQVSLSCAVKGF |
| | | YPSDIAVEWESNGQPENNYKTTPPVLDSDGSFFL |
| | | VSKLTVDKSRWQQGNVFSCSVMHEALHNHYTQ |
| | | KSLSLSPGK |
| P1AH6813 hole chain | 86 | QVQLQQSGGGLVQPGGSLTLSCVASGSILEMSRM |
| | | AWYRQAPGKMRELVASITSIGSIVYADSVKGRFT |
| | | LSRDNTKNTVYLQMNSLKPEDTAVYRCNLLGTW |
| | | SDETYWGQGTQVTVSSTSGSGGGGSGGGGSGGG |
| | | GSGGGGSGGGGEVQLLESGGGLVQPGGSLRLSC |
| | | AASGFSFSSYTMSWVRQAPGKGLEWVATISGGG |
| | | RDIYYPDSVKGRFTISRDNSKNTLYLQMNSLRAE |
| | | DTAVYYCVLLTGRVYFALDSWGQGTLVTVSSAS |
| | | TKGPSVFPLAPSSKSTSGGTAALGCLVKDYFPEPV |
| | | TVSWNSGALTSGVHTFPAVLQSSGLYSLSSVVTV |
| | | PSSSLGTQTYICNVNHKPSNTKVDKKVEPKSCDK |
| | | THTCPPCPAPEAAGGPSVFLFPPKPKDTLMISRTPE |
| | | VTCVVVDVSHEDPEVKFNWYVDGVEVHNAKTK |
| | | PREEQYNSTYRVVSVLTVLHQDWLNGKEYKCKV |
| | | SNKALGAPIEKTISKAKGQPREPQVCTLPPSRDEL |
| | | TKNQVSLSCAVKGFYPSDIAVEWESNGQPENNYK |
| | | TTPPVLDSDGSFFLVSKLTVDKSRWQQGNVFSCS |
| | | VMHEALHNHYTQKSLSLSPG |
| P1AH6850 LC | 87 | QVQLQESGGGLVQAGGSLRLSCAASISISKIDLMG |
| | | WYRQAPGRQRELVARVTNGGDSYYSTSVKGRFT |
| | | ISRDNAKNTLYLQMNSLKPEDTAVYYCYGVPESL |
| | | AAFHWGQGTLVTVSSTSGSGGGGSGGGGSGGGG |
| | | SGGGGSGGGGDIQMTQSPSSLSASVGDRVTITCR |
| | | ASQSISSYLNWYQQKPGKAPKLLIYTASSLQSGVP |
| | | SRFSGSGSGTDFTLTISSLQPEDFATYYCQQSYSTP |
| | | LTFGGGTKVEIKRTVAAPSVFIFPPSDEQLKSGTAS |
| | | VVCLLNNFYPREAKVQWKVDNALQSGNSQESVT |
| | | EQDSKDSTYSLSSTLTLSKADYEKHKVYACEVTH |
| | | QGLSSPVTKSFNRGEC |
| P1AH6850, P1AK2799, | 88 | EVQLLESGGGLVQPGGSLRLSCAASGFTFSSYAM |
| P1AK2798 knob chain | | SWVRQAPGKGLEWVSAITGSGGSTYYADSVKGR |
| | | FTISRDNSRNTLYLQMNSLRAEDTAVYYCAKGEG |
| | | YAGSSYFRASDIWGQGTMVTVSSASTKGPSVFPL |
| | | APSSKSTSGGTAALGCLVKDYFPEPVTVSWNSGA |
| | | LTSGVHTFPAVLQSSGLYSLSSVVTVPSSSLGTQT |
| | | YICNVNHKPSNTKVDKKVEPKSCDKTHTCPPCPA |
| | | PEAAGGPSVFLFPPKPKDTLMISRTPEVTCVVVDV |
| | | SHEDPEVKFNWYVDGVEVHNAKTKPREEQYNST |
| | | YRVVSVLTVLHQDWLNGKEYKCKVSNKALGAPI |
| | | EKTISKAKGQPREPQVYTLPPCRDELTKNQVSLW |
| | | CLVKGFYPSDIAVEWESNGQPENNYKTTPPVLDS |
| | | DGSFFLYSKLTVDKSRWQQGNVFSCSVMHEALH |
| | | NHYTQKSLSLSPG |
| P1AH6814, P1AK2599, | 89 | DIQMTQSPSSLSASVGDRVTITCRASQSISSYLNW |
| P1AK2806 LC | | YQQKPGKAPKLLIYTASSLQSGVPSRFSGSGSGTD |
| | | FTLTISSLQPEDFATYYCQQSYSTPLTFGGGTKVEI |
| | | KRTVAAPSVFIFPPSDEQLKSGTASVVCLLNNFYP |
| | | REAKVQWKVDNALQSGNSQESVTEQDSKDSTYS |
| | | LSSTLTLSKADYEKHKVYACEVTHQGLSSPVTKS |
| | | FNRGEC |
| P1AH6814 knob chain | 90 | QVQLQESGGGLVQAGGSLRLSCAASISISKIDLMG |
| | | WYRQAPGRQRELVARVTNGGDSYYSTSVKGRFT |
| | | ISRDNAKNTLYLQMNSLKPEDTAVYYCYGVPESL |
| | | AAFHWGQGTLVTVSSTSGSGGGGSGGGGSGGGG |
| | | SGGGGSGGGGEVQLLESGGGLVQPGGSLRLSCA |
| | | ASGFTFSSYAMSWVRQAPGKGLEWVSAITGSGGS |
| | | TYYADSVKGRFTISRDNSRNTLYLQMNSLRAEDT |
| | | AVYYCAKGEGYAGSSYFRASDIWGQGTMVTVSS |
| | | ASTKGPSVFPLAPSSKSTSGGTAALGCLVKDYFPE |
| | | PVTVSWNSGALTSGVHTFPAVLQSSGLYSLSSVV |
| | | TVPSSSLGTQTYICNVNHKPSNTKVDKKVEPKSC |
| | | DKTHTCPPCPAPEAAGGPSVFLFPPKPKDTLMISR |
| | | TPEVTCVVVDVSHEDPEVKFNWYVDGVEVHNA |
| | | KTKPREEQYNSTYRVVSVLTVLHQDWLNGKEYK |
| | | CKVSNKALGAPIEKTISKAKGQPREPQVYTLPPCR |
| | | DELTKNQVSLWCLVKGFYPSDIAVEWESNGQPE |
| | | NNYKTTPPVLDSDGSFFLYSKLTVDKSRWQQGN |
| | | VFSCSVMHEALHNHYTQKSLSLSPG |
| P1AI1593 LC | 91 | QVQLQQSGGGLVQPGGSLTLSCVASGSILEMSRM |
| | | AWYRQAPGKMRELVASITSIGSIVYADSVKGRFT |
| | | LSRDNTKNTVYLQMNSLKPEDTAVYRCNLLGTW |
| | | SDETYWGQGTQVTVSSTSGSGGGGSGGGGSGGG |
| | | GSGGGGSGGGGDIVMTQSPDSLAVSLGERATINC |
| | | KASESVDTSDNSFIHWYQQKPGQSPKLLIYRSSTL |
| | | ESGVPDRFSGSGSGTDFTLTISSLQAEDVAVYYCQ |
| | | QNYDVPWTFGQGTKVEIKRTVAAPSVFIFPPSDEQ |
| | | LKSGTASVVCLLNNFYPREAKVQWKVDNALQSG |
| | | NSQESVTEQDSKDSTYSLSSTLTLSKADYEKHKV |
| | | YACEVTHQGLSSPVTKSFNRGEC |
| P1AI1593 hole chain | 92 | EVQLLESGGGLVQPGGSLRLSCAASGFSFSSYTMS |
| | | WVRQAPGKGLEWVATISGGGRDIYYPDSVKGRF |
| | | TISRDNSKNTLYLQMNSLRAEDTAVYYCVLLTGR |
| | | VYFALDSWGQGTLVTVSSASTKGPSVFPLAPSSK |
| | | STSGGTAALGCLVKDYFPEPVTVSWNSGALTSGV |
| | | HTFPAVLQSSGLYSLSSVVTVPSSSLGTQTYICNV |
| | | NHKPSNTKVDKKVEPKSCDKTHTCPPCPAPEAAG |
| | | GPSVFLFPPKPKDTLMISRTPEVTCVVVDVSHEDP |
| | | EVKFNWYVDGVEVHNAKTKPREEQYNSTYRVVS |
| | | VLTVLHQDWLNGKEYKCKVSNKALGAPIEKTISK |
| | | AKGQPREPQVCTLPPSRDELTKNQVSLSCAVKGF |
| | | YPSDIAVEWESNGQPENNYKTTPPVLDSDGSFFL |
| | | VSKLTVDKSRWQQGNVFSCSVMHEALHNHYTQ |
| | | KSLSLSPG |
| PD-1 binder 3 7G12 VH | 93 | EVQLLESGGGLVQPGGSLRLSCAASGFTFSSYWM |
| | | SWVRQAPGKGLEWVSAISGSGGSRYYAESVKGR |
| | | FTISRDNSKNTLYLQMNSLRAEDTAVYYCASSPL |
| | | QWIDVWGQGTTVTVSS |
| PD-1 binder 3 7G12 VL | 94 | DIQMTQSPSSLSASVGDRVTITCRASQGISSWLAW |
| | | YQQKPGKAPKLLIYEASSLQSGVPSRFSGSGSGTD |
| | | FTLTISSLQPEDFATYYCQQANQFPFTFGPGTKVDI |
| | | K |
| P1AK2802 LC | 95 | QVQLQQSGGGLVQPGGSLTLSCVASGSILEMSRM |
| | | AWYRQAPGKMRELVASITSIGSIVYADSVKGRFT |
| | | LSRDNTKNTVYLQMNSLKPEDTAVYRCNLLGTW |
| | | SDETYWGQGTQVTVSSGGSGGGGSGGGGSGGGG |
| | | SGGGGSGGDIQMTQSPSSLSASVGDRVTITCRASQ |
| | | GISSWLAWYQQKPGKAPKLLIYEASSLQSGVPSR |
| | | FSGSGSGTDFTLTISSLQPEDFATYYCQQANQFPF |
| | | TFGPGTKVDIKRTVAAPSVFIFPPSDEQLKSGTAS |
| | | VVCLLNNFYPREAKVQWKVDNALQSGNSQESVT |
| | | EQDSKDSTYSLSSTLTLSKADYEKHKVYACEVTH |
| | | QGLSSPVTKSFNRGEC |
| P1AK2802, P1AK2803 | 96 | EVQLLESGGGLVQPGGSLRLSCAASGFTFSSYWM |
| knob chain | | SWVRQAPGKGLEWVSAISGSGGSRYYAESVKGR |
| | | FTISRDNSKNTLYLQMNSLRAEDTAVYYCASSPL |
| | | QWIDVWGQGTTVTVSSASTKGPSVFPLAPSSKST |
| | | SGGTAALGCLVKDYFPEPVTVSWNSGALTSGVH |
| | | TFPAVLQSSGLYSLSSVVTVPSSSLGTQTYICNVN |
| | | HKPSNTKVDKKVEPKSCDKTHTCPPCPAPEAAGG |
| | | PSVFLFPPKPKDTLMISRTPEVTCVVVDVSHEDPE |
| | | VKFNWYVDGVEVHNAKTKPREEQYNSTYRVVS |
| | | VLTVLHQDWLNGKEYKCKVSNKALGAPIEKTISK |
| | | AKGQPREPQVYTLPPCRDELTKNQVSLWCLVKG |
| | | FYPSDIAVEWESNGQPENNYKTTPPVLDSDGSFFL |
| | | YSKLTVDKSRWQQGNVFSCSVMHEALHNHYTQ |
| | | KSLSLSPG |
| P1AK2803 LC | 97 | QVQLQESGGGLVQAGGSLRLSCAASISISKIDLMG |
| | | WYRQAPGRQRELVARVTNGGDSYYSTSVKGRFT |
| | | ISRDNAKNTLYLQMNSLKPEDTAVYYCYGVPESL |
| | | AAFHWGQGTLVTVSSGGSGGGGSGGGGSGGGGS |
| | | GGGGSGGDIQMTQSPSSLSASVGDRVTITCRASQ |
| | | GISSWLAWYQQKPGKAPKLLIYEASSLQSGVPSR |
| | | FSGSGSGTDFTLTISSLQPEDFATYYCQQANQFPF |
| | | TFGPGTKVDIKRTVAAPSVFIFPPSDEQLKSGTAS |
| | | VVCLLNNFYPREAKVQWKVDNALQSGNSQESVT |
| | | EQDSKDSTYSLSSTLTLSKADYEKHKVYACEVTH |
| | | QGLSSPVTKSFNRGEC |
| LC (P1AK2809, | 98 | DIQMTQSPSSLSASVGDRVTITCRASQGISSWLAW |
| P1AK2810) | | YQQKPGKAPKLLIYEASSLQSGVPSRFSGSGSGTD |
| | | FTLTISSLQPEDFATYYCQQANQFPFTFGPGTKVDI |
| | | KRTVAAPSVFIFPPSDEQLKSGTASVVCLLNNFYP |
| | | REAKVQWKVDNALQSGNSQESVTEQDSKDSTYS |
| | | LSSTLTLSKADYEKHKVYACEVTHQGLSSPVTKS |
| | | FNRGEC |
| P1AK2809 knob chain | 99 | QVQLQQSGGGLVQPGGSLTLSCVASGSILEMSRM |
| | | AWYRQAPGKMRELVASITSIGSIVYADSVKGRFT |
| | | LSRDNTKNTVYLQMNSLKPEDTAVYRCNLLGTW |
| | | SDETYWGQGTQVTVSSGGSGGGGSGGGGSGGGG |
| | | SGGGGSGGEVQLLESGGGLVQPGGSLRLSCAASG |
| | | FTFSSYWMSWVRQAPGKGLEWVSAISGSGGSRY |
| | | YAESVKGRFTISRDNSKNTLYLQMNSLRAEDTAV |
| | | YYCASSPLQWIDVWGQGTTVTVSSASTKGPSVFP |
| | | LAPSSKSTSGGTAALGCLVKDYFPEPVTVSWNSG |
| | | ALTSGVHTFPAVLQSSGLYSLSSVVTVPSSSLGTQ |
| | | TYICNVNHKPSNTKVDKKVEPKSCDKTHTCPPCP |
| | | APEAAGGPSVFLFPPKPKDTLMISRTPEVTCVVVD |
| | | VSHEDPEVKFNWYVDGVEVHNAKTKPREEQYNS |
| | | TYRVVSVLTVLHQDWLNGKEYKCKVSNKALGA |
| | | PIEKTISKAKGQPREPQVYTLPPCRDELTKNQVSL |
| | | WCLVKGFYPSDIAVEWESNGQPENNYKTTPPVLD |
| | | SDGSFFLYSKLTVDKSRWQQGNVFSCSVMHEAL |
| | | HNHYTQKSLSLSPG |
| P1AK2810 knob chain | 100 | QVQLQESGGGLVQAGGSLRLSCAASISISKIDLMG |
| | | WYRQAPGRQRELVARVTNGGDSYYSTSVKGRFT |
| | | ISRDNAKNTLYLQMNSLKPEDTAVYYCYGVPESL |
| | | AAFHWGQGTLVTVSSGGSGGGGSGGGGSGGGGS |
| | | GGGGSGGEVQLLESGGGLVQPGGSLRLSCAASGF |
| | | TFSSYWMSWVRQAPGKGLEWVSAISGSGGSRYY |
| | | AESVKGRFTISRDNSKNTLYLQMNSLRAEDTAVY |
| | | YCASSPLQWIDVWGQGTTVTVSSASTKGPSVFPL |
| | | APSSKSTSGGTAALGCLVKDYFPEPVTVSWNSGA |
| | | LTSGVHTFPAVLQSSGLYSLSSVVTVPSSSLGTQT |
| | | YICNVNHKPSNTKVDKKVEPKSCDKTHTCPPCPA |
| | | PEAAGGPSVFLFPPKPKDTLMISRTPEVTCVVVDV |
| | | SHEDPEVKFNWYVDGVEVHNAKTKPREEQYNST |
| | | YRVVSVLTVLHQDWLNGKEYKCKVSNKALGAPI |
| | | EKTISKAKGQPREPQVYTLPPCRDELTKNQVSLW |
| | | CLVKGFYPSDIAVEWESNGQPENNYKTTPPVLDS |
| | | DGSFFLYSKLTVDKSRWQQGNVFSCSVMHEALH |
| | | NHYTQKSLSLSPG |
| P1AK2599 knob chain | 101 | QVQLQESGGGLVQAGGSLRLSCAASISISKIDLMG |
| | | WYRQAPGRQRELVARVTNGGDSYYSTSVKGRFT |
| | | ISRDNAKNTLYLQMNSLKPEDTAVYYCYGVPESL |
| | | AAFHWGQGTLVTVSSGGSGGGGSGGGGSGGGGS |
| | | GGGGSGGEVQLLESGGGLVQPGGSLRLSCAASGF |
| | | TFSSYAMSWVRQAPGKGLEWVSAITGSGGSTYY |
| | | ADSVKGRFTISRDNSRNTLYLQMNSLRAEDTAVY |
| | | YCAKGEGYAGSSYFRASDIWGQGTMVTVSSAST |
| | | KGPSVFPLAPSSKSTSGGTAALGCLVKDYFPEPVT |
| | | VSWNSGALTSGVHTFPAVLQSSGLYSLSSVVTVP |
| | | SSSLGTQTYICNVNHKPSNTKVDKKVEPKSCDKT |
| | | HTCPPCPAPEAAGGPSVFLFPPKPKDTLMISRTPE |
| | | VTCVVVDVSHEDPEVKFNWYVDGVEVHNAKTK |
| | | PREEQYNSTYRVVSVLTVLHQDWLNGKEYKCKV |
| | | SNKALGAPIEKTISKAKGQPREPQVYTLPPCRDEL |
| | | TKNQVSLWCLVKGFYPSDIAVEWESNGQPENNY |
| | | KTTPPVLDSDGSFFLYSKLTVDKSRWQQGNVFSC |
| | | SVMHEALHNHYTQKSLSLSPG |
| P1AK2799 LC | 102 | QVQLQESGGGLVQAGGSLRLSCAASISISKIDLMG |
| | | WYRQAPGRQRELVARVTNGGDSYYSTSVKGRFT |
| | | ISRDNAKNTLYLQMNSLKPEDTAVYYCYGVPESL |
| | | AAFHWGQGTLVTVSSGGSGGGGSGGGGSGGGGS |
| | | GGGGSGGDIQMTQSPSSLSASVGDRVTITCRASQS |
| | | ISSYLNWYQQKPGKAPKLLIYTASSLQSGVPSRFS |
| | | GSGSGTDFTLTISSLQPEDFATYYCQQSYSTPLTFG |
| | | GGTKVEIKRTVAAPSVFIFPPSDEQLKSGTASVVC |
| | | LLNNFYPREAKVQWKVDNALQSGNSQESVTEQD |
| | | SKDSTYSLSSTLTLSKADYEKHKVYACEVTHQGL |
| | | SSPVTKSFNRGEC |
| P1AK2806 knob chain | 103 | QVQLQQSGGGLVQPGGSLTLSCVASGSILEMSRM |
| | | AWYRQAPGKMRELVASITSIGSIVYADSVKGRFT |
| | | LSRDNTKNTVYLQMNSLKPEDTAVYRCNLLGTW |
| | | SDETYWGQGTQVTVSSGGSGGGGSGGGGSGGGG |
| | | SGGGGSGGEVQLLESGGGLVQPGGSLRLSCAASG |
| | | FTFSSYAMSWVRQAPGKGLEWVSAITGSGGSTY |
| | | YADSVKGRFTISRDNSRNTLYLQMNSLRAEDTAV |
| | | YYCAKGEGYAGSSYFRASDIWGQGTMVTVSSAS |
| | | TKGPSVFPLAPSSKSTSGGTAALGCLVKDYFPEPV |
| | | TVSWNSGALTSGVHTFPAVLQSSGLYSLSSVVTV |
| | | PSSSLGTQTYICNVNHKPSNTKVDKKVEPKSCDK |
| | | THTCPPCPAPEAAGGPSVFLFPPKPKDTLMISRTPE |
| | | VTCVVVDVSHEDPEVKFNWYVDGVEVHNAKTK |
| | | PREEQYNSTYRVVSVLTVLHQDWLNGKEYKCKV |
| | | SNKALGAPIEKTISKAKGQPREPQVYTLPPCRDEL |
| | | TKNQVSLWCLVKGFYPSDIAVEWESNGQPENNY |
| | | KTTPPVLDSDGSFFLYSKLTVDKSRWQQGNVFSC |
| | | SVMHEALHNHYTQKSLSLSPG |
| P1AK2798 LC | 104 | QVQLQQSGGGLVQPGGSLTLSCVASGSILEMSRM |
| | | AWYRQAPGKMRELVASITSIGSIVYADSVKGRFT |
| | | LSRDNTKNTVYLQMNSLKPEDTAVYRCNLLGTW |
| | | SDETYWGQGTQVTVSSGGSGGGGSGGGGSGGGG |
| | | SGGGGSGGDIQMTQSPSSLSASVGDRVTITCRASQ |
| | | SISSYLNWYQQKPGKAPKLLIYTASSLQSGVPSRF |
| | | SGSGSGTDFTLTISSLQPEDFATYYCQQSYSTPLTF |
| | | GGGTKVEIKRTVAAPSVFIFPPSDEQLKSGTASVV |
| | | CLLNNFYPREAKVQWKVDNALQSGNSQESVTEQ |
| | | DSKDSTYSLSSTLTLSKADYEKHKVYACEVTHQG |
| | | LSSPVTKSFNRGEC |
| P1AJ5092 LC | 105 | QVQLQQSGGGLVQPGGSLTLSCVASGSILEMSRM |
| | | AWYRQAPGKMRELVASITSIGSIVYADSVKGRFT |
| | | LSRDNTKNTVYLQMNSLKPEDTAVYRCNLLGTW |
| | | SDETYWGQGTQVTVSSGGSGGGGSGGGGSGGGG |
| | | SGGGGSGGEIVLTQSPGTLSLSPGERATLSCRASQ |
| | | SVTSSYLAWYQQKPGQAPRLLINVGSRRATGIPD |
| | | RFSGSGSGTDFTLTISRLEPEDFAVYYCQQGIMLP |
| | | PTFGQGTKVEIKRTVAAPSVFIFPPSDEQLKSGTAS |
| | | VVCLLNNFYPREAKVQWKVDNALQSGNSQESVT |
| | | EQDSKDSTYSLSSTLTLSKADYEKHKVYACEVTH |
| | | QGLSSPVTKSFNRGEC |
| hole chain (P1AJ5092- | 106 | DKTHTCPPCPAPEAAGGPSVFLFPPKPKDTLMASR |
| P1AJ5099, P1AJ4165- | | TPEVTCVVVDVSHEDPEVKFNWYVDGVEVHNA |
| P1AJ4180, P1AJ5654- | | KTKPREEQYNSTYRVVSVLTVLAQDWLNGKEYK |
| P1AJ5661, P1AL0544- | | CKVSNKALGAPIEKTISKAKGQPREPQVCTLPPSR |
| P1AL0556, P1AK5514 - | | DELTKNQVSLSCAVKGFYPSDIAVEWESNGQPEN |
| P1AK5521) | | NYKTTPPVLDSDGSFFLVSKLTVDKSRWQQGNVF |
| | | SCSVMHEALHNAYTQKSLSLSPG |
| P1AJ5093 LC | 107 | QVQLQESGGGLVQAGGSLRLSCAASISISKIDLMG |
| | | WYRQAPGRQRELVARVTNGGDSYYSTSVKGRFT |
| | | ISRDNAKNTLYLQMNSLKPEDTAVYYCYGVPESL |
| | | AAFHWGQGTLVTVSSGGSGGGGSGGGGSGGGGS |
| | | GGGGSGGEIVLTQSPGTLSLSPGERATLSCRASQS |
| | | VTSSYLAWYQQKPGQAPRLLINVGSRRATGIPDR |
| | | FSGSGSGTDFTLTISRLEPEDFAVYYCQQGIMLPPT |
| | | FGQGTKVEIKRTVAAPSVFIFPPSDEQLKSGTASV |
| | | VCLLNNFYPREAKVQWKVDNALQSGNSQESVTE |
| | | QDSKDSTYSLSSTLTLSKADYEKHKVYACEVTHQ |
| | | GLSSPVTKSFNRGEC |
| P1AJ5094 LC | 108 | QVQLQQSGGGLVQPGGSLTLSCVASGSILEMSRM |
| | | AWYRQAPGKMRELVASITSIGSIVYADSVKGRFT |
| | | LSRDNTKNTVYLQMNSLKPEDTAVYRCNLLGTW |
| | | SDETYWGQGTQVTVSSGGSGGGGSGGGGSGGGG |
| | | SGGGGSGGEIVLTQSPATLSLSPGERATLSCRASE |
| | | SVDNYGLSFINWFQQKPGQAPRLLIYGTSNRGSGI |
| | | PARFSGSGSGTDFTLTISSLEPEDFAVYFCQQSNEV |
| | | PYTFGGGTKVEIKRTVAAPSVFIFPPSDEQLKSGT |
| | | ASVVCLLNNFYPREAKVQWKVDNALQSGNSQES |
| | | VTEQDSKDSTYSLSSTLTLSKADYEKHKVYACEV |
| | | THQGLSSPVTKSFNRGEC |
| P1AJ5095 LC | 109 | QVQLQESGGGLVQAGGSLRLSCAASISISKIDLMG |
| | | WYRQAPGRQRELVARVTNGGDSYYSTSVKGRFT |
| | | ISRDNAKNTLYLQMNSLKPEDTAVYYCYGVPESL |
| | | AAFHWGQGTLVTVSSGGSGGGGSGGGGSGGGGS |
| | | GGGGSGGEIVLTQSPATLSLSPGERATLSCRASES |
| | | VDNYGLSFINWFQQKPGQAPRLLIYGTSNRGSGIP |
| | | ARFSGSGSGTDFTLTISSLEPEDFAVYFCQQSNEVP |
| | | YTFGGGTKVEIKRTVAAPSVFIFPPSDEQLKSGTA |
| | | SVVCLLNNFYPREAKVQWKVDNALQSGNSQESV |
| | | TEQDSKDSTYSLSSTLTLSKADYEKHKVYACEVT |
| | | HQGLSSPVTKSFNRGEC |
| P1AJ5096 knob chain | 110 | QVQLQQSGGGLVQPGGSLTLSCVASGSILEMSRM |
| | | AWYRQAPGKMRELVASITSIGSIVYADSVKGRFT |
| | | LSRDNTKNTVYLQMNSLKPEDTAVYRCNLLGTW |
| | | SDETYWGQGTQVTVSSGGSGGGGSGGGGSGGGG |
| | | SGGGGSGGEVQLLESGGGLVQPGGSLRLSCAASG |
| | | FTFSSYAMSWVRQAPGKGLEWVSAIIGSGASTYY |
| | | ADSVKGRFTISRDNSKNTLYLQMNSLRAEDTAVY |
| | | YCAKGWFGGFNYWGQGTLVTVSSASTKGPSVFP |
| | | LAPSSKSTSGGTAALGCLVKDYFPEPVTVSWNSG |
| | | ALTSGVHTFPAVLQSSGLYSLSSVVTVPSSSLGTQ |
| | | TYICNVNHKPSNTKVDKKVEPKSCDKTHTCPPCP |
| | | APEAAGGPSVFLFPPKPKDTLMISRTPEVTCVVVD |
| | | VSHEDPEVKFNWYVDGVEVHNAKTKPREEQYNS |
| | | TYRVVSVLTVLHQDWLNGKEYKCKVSNKALGA |
| | | PIEKTISKAKGQPREPQVYTLPPCRDELTKNQVSL |
| | | WCLVKGFYPSDIAVEWESNGQPENNYKTTPPVLD |
| | | SDGSFFLYSKLTVDKSRWQQGNVFSCSVMHEAL |
| | | HNHYTQKSLSLSPG |
| P1AJ5097 knob chain | 111 | QVQLQESGGGLVQAGGSLRLSCAASISISKIDLMG |
| | | WYRQAPGRQRELVARVTNGGDSYYSTSVKGRFT |
| | | ISRDNAKNTLYLQMNSLKPEDTAVYYCYGVPESL |
| | | AAFHWGQGTLVTVSSGGSGGGGSGGGGSGGGGS |
| | | GGGGSGGEVQLLESGGGLVQPGGSLRLSCAASGF |
| | | TFSSYAMSWVRQAPGKGLEWVSAIIGSGASTYYA |
| | | DSVKGRFTISRDNSKNTLYLQMNSLRAEDTAVYY |
| | | CAKGWFGGFNYWGQGTLVTVSSASTKGPSVFPL |
| | | APSSKSTSGGTAALGCLVKDYFPEPVTVSWNSGA |
| | | LTSGVHTFPAVLQSSGLYSLSSVVTVPSSSLGTQT |
| | | YICNVNHKPSNTKVDKKVEPKSCDKTHTCPPCPA |
| | | PEAAGGPSVFLFPPKPKDTLMISRTPEVTCVVVDV |
| | | SHEDPEVKFNWYVDGVEVHNAKTKPREEQYNST |
| | | YRVVSVLTVLHQDWLNGKEYKCKVSNKALGAPI |
| | | EKTISKAKGQPREPQVYTLPPCRDELTKNQVSLW |
| | | CLVKGFYPSDIAVEWESNGQPENNYKTTPPVLDS |
| | | DGSFFLYSKLTVDKSRWQQGNVFSCSVMHEALH |
| | | NHYTQKSLSLSPG |
| P1AJ5098 knob chain | 112 | QVQLQQSGGGLVQPGGSLTLSCVASGSILEMSRM |
| | | AWYRQAPGKMRELVASITSIGSIVYADSVKGRFT |
| | | LSRDNTKNTVYLQMNSLKPEDTAVYRCNLLGTW |
| | | SDETYWGQGTQVTVSSGGSGGGGSGGGGSGGGG |
| | | SGGGGSGGQVQLVQSGAEVKKPGASVKVSCKAS |
| | | GYTLTDYNMDWVRQAPGQGLEWIGDIYPNTGGT |
| | | IYNQKFKGRVTMTIDTSTSTVYMELSSLRSEDTAV |
| | | YYCTRFRGIHYAMDYWGQGTTVTVSSASTKGPS |
| | | VFPLAPSSKSTSGGTAALGCLVKDYFPEPVTVSW |
| | | NSGALTSGVHTFPAVLQSSGLYSLSSVVTVPSSSL |
| | | GTQTYICNVNHKPSNTKVDKKVEPKSCDKTHTCP |
| | | PCPAPEAAGGPSVFLFPPKPKDTLMISRTPEVTCV |
| | | VVDVSHEDPEVKFNWYVDGVEVHNAKTKPREE |
| | | QYNSTYRVVSVLTVLHQDWLNGKEYKCKVSNK |
| | | ALGAPIEKTISKAKGQPREPQVYTLPPCRDELTKN |
| | | QVSLWCLVKGFYPSDIAVEWESNGQPENNYKTTP |
| | | PVLDSDGSFFLYSKLTVDKSRWQQGNVFSCSVM |
| | | HEALHNHYTQKSLSLSPG |
| P1AJ5099 knob chain | 113 | QVQLQESGGGLVQAGGSLRLSCAASISISKIDLMG |
| | | WYRQAPGRQRELVARVTNGGDSYYSTSVKGRFT |
| | | ISRDNAKNTLYLQMNSLKPEDTAVYYCYGVPESL |
| | | AAFHWGQGTLVTVSSGGSGGGGGGGGSGGGGS |
| | | GGGGSGGQVQLVQSGAEVKKPGASVKVSCKASG |
| | | YTLTDYNMDWVRQAPGQGLEWIGDIYPNTGGTI |
| | | YNQKFKGRVTMTIDTSTSTVYMELSSLRSEDTAV |
| | | YYCTRFRGIHYAMDYWGQGTTVTVSSASTKGPS |
| | | VFPLAPSSKSTSGGTAALGCLVKDYFPEPVTVSW |
| | | NSGALTSGVHTFPAVLQSSGLYSLSSVVTVPSSSL |
| | | GTQTYICNVNHKPSNTKVDKKVEPKSCDKTHTCP |
| | | PCPAPEAAGGPSVFLFPPKPKDTLMISRTPEVTCV |
| | | VVDVSHEDPEVKFNWYVDGVEVHNAKTKPREE |
| | | QYNSTYRVVSVLTVLHQDWLNGKEYKCKVSNK |
| | | ALGAPIEKTISKAKGQPREPQVYTLPPCRDELTKN |
| | | QVSLWCLVKGFYPSDIAVEWESNGQPENNYKTTP |
| | | PVLDSDGSFFLYSKLTVDKSRWQQGNVFSCSVM |
| | | HEALHNHYTQKSLSLSPG |
| EGFR binder 1 VH | 114 | QVQLVQSGAEVKKPGSSVKVSCKASGFTFTDYKI |
| | | HWVRQAPGQGLEWMGYFNPNSGYSTYAQKFQG |
| | | RVTITADKSTSTAYMELSSLRSEDTAVYYCARLSP |
| | | GGYYVMDAWGQGTTVTVSS |
| EGFR binder 1 VL | 115 | DIQMTQSPSSLSASVGDRVTITCRASQGINNYLNW |
| | | YQQKPGKAPKRLIYNTNNLQTGVPSRFSGSGSGT |
| | | EFTLTISSLQPEDFATYYCLQHNSFPTFGQGTKLEI |
| | | K |
| EGFR binder 2 VH | 116 | QVQLKQSGPGLVQPSQSLSITCTVSGFSLTNYGVH |
| | | WVRQSPGKGLEWLGVIWSGGNTDYNTPFTSRLSI |
| | | NKDNSKSQVFFKMNSLQSNDTAIYYCARALTYY |
| | | DYEFAYWGQGTLVTVSA |
| EGFR binder 2 VL | 117 | DILLTQSPVILSVSPGERVSFSCRASQSIGTNIHWY |
| | | QQRTNGSPRLLIKYASESISGIPSRFSGSGSGTDFTL |
| | | SINSVESEDIADYYCQQNNNWPTTFGAGTKLELK |
| P1AJ4165 LC | 118 | QVQLQQSGGGLVQPGGSLTLSCVASGSILEMSRM |
| | | AWYRQAPGKMRELVASITSIGSIVYADSVKGRFT |
| | | LSRDNTKNTVYLQMNSLKPEDTAVYRCNLLGTW |
| | | SDETYWGQGTQVTVSSGGSGGGGSGGGGSGGGG |
| | | SGGGGSGGDIQMTQSPSSLSASVGDRVTITCRASQ |
| | | GINNYLNWYQQKPGKAPKRLIYNTNNLQTGVPS |
| | | RFSGSGSGTEFTLTISSLQPEDFATYYCLQHNSFPT |
| | | FGQGTKLEIKRTVAAPSVFIFPPSDEQLKSGTASV |
| | | VCLLNNFYPREAKVQWKVDNALQSGNSQESVTE |
| | | QDSKDSTYSLSSTLTLSKADYEKHKVYACEVTHQ |
| | | GLSSPVTKSFNRGEC |
| knob chain (P1AJ4165, | 119 | QVQLVQSGAEVKKPGSSVKVSCKASGFTFTDYKI |
| P1AJ4166, P1AK5516, | | HWVRQAPGQGLEWMGYFNPNSGYSTYAQKFQG |
| P1AK5517) | | RVTITADKSTSTAYMELSSLRSEDTAVYYCARLSP |
| | | GGYYVMDAWGQGTTVTVSSASTKGPSVFPLAPS |
| | | SKSTSGGTAALGCLVKDYFPEPVTVSWNSGALTS |
| | | GVHTFPAVLQSSGLYSLSSVVTVPSSSLGTQTYIC |
| | | NVNHKPSNTKVDKKVEPKSCDKTHTCPPCPAPEA |
| | | AGGPSVFLFPPKPKDTLMISRTPEVTCVVVDVSHE |
| | | DPEVKFNWYVDGVEVHNAKTKPREEQYNSTYRV |
| | | VSVLTVLHQDWLNGKEYKCKVSNKALGAPIEKTI |
| | | SKAKGQPREPQVYTLPPCRDELTKNQVSLWCLV |
| | | KGFYPSDIAVEWESNGQPENNYKTTPPVLDSDGS |
| | | FFLYSKLTVDKSRWQQGNVFSCSVMHEALHNHY |
| | | TQKSLSLSPG |
| P1AJ4166 LC | 120 | QVQLQESGGGLVQAGGSLRLSCAASISISKIDLMG |
| | | WYRQAPGRQRELVARVTNGGDSYYSTSVKGRFT |
| | | ISRDNAKNTLYLQMNSLKPEDTAVYYCYGVPESL |
| | | AAFHWGQGTLVTVSSGGSGGGGSGGGGSGGGGS |
| | | GGGGSGGDIQMTQSPSSLSASVGDRVTITCRASQ |
| | | GINNYLNWYQQKPGKAPKRLIYNTNNLQTGVPS |
| | | RFSGSGSGTEFTLTISSLQPEDFATYYCLQHNSFPT |
| | | FGQGTKLEIKRTVAAPSVFIFPPSDEQLKSGTASV |
| | | VCLLNNFYPREAKVQWKVDNALQSGNSQESVTE |
| | | QDSKDSTYSLSSTLTLSKADYEKHKVYACEVTHQ |
| | | GLSSPVTKSFNRGEC |
| P1AJ4167 LC | 121 | QVQLQQSGGGLVQPGGSLTLSCVASGSILEMSRM |
| | | AWYRQAPGKMRELVASITSIGSIVYADSVKGRFT |
| | | LSRDNTKNTVYLQMNSLKPEDTAVYRCNLLGTW |
| | | SDETYWGQGTQVTVSSGGSGGGGSGGGGSGGGG |
| | | SGGGGSGGDILLTQSPVILSVSPGERVSFSCRASQS |
| | | IGTNIHWYQQRTNGSPRLLIKYASESISGIPSRFSGS |
| | | GSGTDFTLSINSVESEDIADYYCQQNNNWPTTFG |
| | | AGTKLELKRTVAAPSVFIFPPSDEQLKSGTASVVC |
| | | LLNNFYPREAKVQWKVDNALQSGNSQESVTEQD |
| | | SKDSTYSLSSTLTLSKADYEKHKVYACEVTHQGL |
| | | SSPVTKSFNRGEC |
| knob chain (P1AJ4167, | 122 | QVQLKQSGPGLVQPSQSLSITCTVSGFSLTNYGVH |
| P1AJ4168, P1AK5514, | | WVRQSPGKGLEWLGVIWSGGNTDYNTPFTSRLSI |
| P1AK5515) | | NKDNSKSQVFFKMNSLQSNDTAIYYCARALTYY |
| | | DYEFAYWGQGTLVTVSAASTKGPSVFPLAPSSKS |
| | | TSGGTAALGCLVKDYFPEPVTVSWNSGALTSGV |
| | | HTFPAVLQSSGLYSLSSVVTVPSSSLGTQTYICNV |
| | | NHKPSNTKVDKKVEPKSCDKTHTCPPCPAPEAAG |
| | | GPSVFLFPPKPKDTLMISRTPEVTCVVVDVSHEDP |
| | | EVKFNWYVDGVEVHNAKTKPREEQYNSTYRVVS |
| | | VLTVLHQDWLNGKEYKCKVSNKALGAPIEKTISK |
| | | AKGQPREPQVYTLPPCRDELTKNQVSLWCLVKG |
| | | FYPSDIAVEWESNGQPENNYKTTPPVLDSDGSFFL |
| | | YSKLTVDKSRWQQGNVFSCSVMHEALHNHYTQ |
| | | KSLSLSPG |
| P1AJ4168 LC | 123 | QVQLQESGGGLVQAGGSLRLSCAASISISKIDLMG |
| | | WYRQAPGRQRELVARVTNGGDSYYSTSVKGRFT |
| | | ISRDNAKNTLYLQMNSLKPEDTAVYYCYGVPESL |
| | | AAFHWGQGTLVTVSSGGSGGGGSGGGGSGGGGS |
| | | GGGGSGGDILLTQSPVILSVSPGERVSFSCRASQSI |
| | | GTNIHWYQQRINGSPRLLIKYASESISGIPSRFSGS |
| | | GSGTDFTLSINSVESEDIADYYCQQNNNWPTTFG |
| | | AGTKLELKRTVAAPSVFIFPPSDEQLKSGTASVVC |
| | | LLNNFYPREAKVQWKVDNALQSGNSQESVTEQD |
| | | SKDSTYSLSSTLTLSKADYEKHKVYACEVTHQGL |
| | | SSPVTKSFNRGEC |
| LC (P1AJ4169, | 124 | DIQMTQSPSSLSASVGDRVTITCRASQGINNYLNW |
| P1AJ4170, P1AK5520, | | YQQKPGKAPKRLIYNTNNLQTGVPSRFSGSGSGT |
| P1AK5521) | | EFTLTISSLQPEDFATYYCLQHNSFPTFGQGTKLEI |
| | | KRTVAAPSVFIFPPSDEQLKSGTASVVCLLNNFYP |
| | | REAKVQWKVDNALQSGNSQESVTEQDSKDSTYS |
| | | LSSTLTLSKADYEKHKVYACEVTHQGLSSPVTKS |
| | | FNRGEC |
| P1AJ4169 knob chain | 125 | QVQLQQSGGGLVQPGGSLTLSCVASGSILEMSRM |
| | | AWYRQAPGKMRELVASITSIGSIVYADSVKGRFT |
| | | LSRDNTKNTVYLQMNSLKPEDTAVYRCNLLGTW |
| | | SDETYWGQGTQVTVSSGGSGGGGSGGGGSGGGG |
| | | SGGGGSGGQVQLVQSGAEVKKPGSSVKVSCKAS |
| | | GFTFTDYKIHWVRQAPGQGLEWMGYFNPNSGYS |
| | | TYAQKFQGRVTITADKSTSTAYMELSSLRSEDTA |
| | | VYYCARLSPGGYYVMDAWGQGTTVTVSSASTK |
| | | GPSVFPLAPSSKSTSGGTAALGCLVKDYFPEPVTV |
| | | SWNSGALTSGVHTFPAVLQSSGLYSLSSVVTVPSS |
| | | SLGTQTYICNVNHKPSNTKVDKKVEPKSCDKTHT |
| | | CPPCPAPEAAGGPSVFLFPPKPKDTLMISRTPEVTC |
| | | VVVDVSHEDPEVKFNWYVDGVEVHNAKTKPRE |
| | | EQYNSTYRVVSVLTVLHQDWLNGKEYKCKVSN |
| | | KALGAPIEKTISKAKGQPREPQVYTLPPCRDELTK |
| | | NQVSLWCLVKGFYPSDIAVEWESNGQPENNYKT |
| | | TPPVLDSDGSFFLYSKLTVDKSRWQQGNVFSCSV |
| | | MHEALHNHYTQKSLSLSPG |
| P1AJ4170 knob chain | 126 | QVQLQESGGGLVQAGGSLRLSCAASISISKIDLMG |
| | | WYRQAPGRQRELVARVTNGGDSYYSTSVKGRFT |
| | | ISRDNAKNTLYLQMNSLKPEDTAVYYCYGVPESL |
| | | AAFHWGQGTLVTVSSGGSGGGGSGGGGSGGGGS |
| | | GGGGSGGQVQLVQSGAEVKKPGSSVKVSCKASG |
| | | FTFTDYKIHWVRQAPGQGLEWMGYFNPNSGYST |
| | | YAQKFQGRVTITADKSTSTAYMELSSLRSEDTAV |
| | | YYCARLSPGGYYVMDAWGQGTTVTVSSASTKGP |
| | | SVFPLAPSSKSTSGGTAALGCLVKDYFPEPVTVS |
| | | WNSGALTSGVHTFPAVLQSSGLYSLSSVVTVPSSS |
| | | LGTQTYICNVNHKPSNTKVDKKVEPKSCDKTHTC |
| | | PPCPAPEAAGGPSVFLFPPKPKDTLMISRTPEVTC |
| | | VVVDVSHEDPEVKFNWYVDGVEVHNAKTKPRE |
| | | EQYNSTYRVVSVLTVLHQDWLNGKEYKCKVSN |
| | | KALGAPIEKTISKAKGQPREPQVYTLPPCRDELTK |
| | | NQVSLWCLVKGFYPSDIAVEWESNGQPENNYKT |
| | | TPPVLDSDGSFFLYSKLTVDKSRWQQGNVFSCSV |
| | | MHEALHNHYTQKSLSLSPG |
| LC (P1AJ4171, | 127 | DILLTQSPVILSVSPGERVSFSCRASQSIGTNIHWY |
| P1AJ4172, P1AK5518, | | QQRTNGSPRLLIKYASESISGIPSRFSGSGSGTDFTL |
| P1AK5519) | | SINSVESEDIADYYCQQNNNWPTTFGAGTKLELK |
| | | RTVAAPSVFIFPPSDEQLKSGTASVVCLLNNFYPR |
| | | EAKVQWKVDNALQSGNSQESVTEQDSKDSTYSL |
| | | SSTLTLSKADYEKHKVYACEVTHQGLSSPVTKSF |
| | | NRGEC |
| P1AJ4171 knob chain | 128 | QVQLQQSGGGLVQPGGSLTLSCVASGSILEMSRM |
| | | AWYRQAPGKMRELVASITSIGSIVYADSVKGRFT |
| | | LSRDNTKNTVYLQMNSLKPEDTAVYRCNLLGTW |
| | | SDETYWGQGTQVTVSSGGSGGGGSGGGGSGGGG |
| | | SGGGGSGGQVQLKQSGPGLVQPSQSLSITCTVSGF |
| | | SLTNYGVHWVRQSPGKGLEWLGVIWSGGNTDY |
| | | NTPFTSRLSINKDNSKSQVFFKMNSLQSNDTAIYY |
| | | CARALTYYDYEFAYWGQGTLVTVSAASTKGPSV |
| | | FPLAPSSKSTSGGTAALGCLVKDYFPEPVTVSWN |
| | | SGALTSGVHTFPAVLQSSGLYSLSSVVTVPSSSLG |
| | | TQTYICNVNHKPSNTKVDKKVEPKSCDKTHTCPP |
| | | CPAPEAAGGPSVFLFPPKPKDTLMISRTPEVTCVV |
| | | VDVSHEDPEVKFNWYVDGVEVHNAKTKPREEQ |
| | | YNSTYRVVSVLTVLHQDWLNGKEYKCKVSNKA |
| | | LGAPIEKTISKAKGQPREPQVYTLPPCRDELTKNQ |
| | | VSLWCLVKGFYPSDIAVEWESNGQPENNYKTTPP |
| | | VLDSDGSFFLYSKLTVDKSRWQQGNVFSCSVMH |
| | | EALHNHYTQKSLSLSPG |
| P1AJ4172 knob chain | 129 | QVQLQESGGGLVQAGGSLRLSCAASISISKIDLMG |
| | | WYRQAPGRQRELVARVTNGGDSYYSTSVKGRFT |
| | | ISRDNAKNTLYLQMNSLKPEDTAVYYCYGVPESL |
| | | AAFHWGQGTLVTVSSGGSGGGGSGGGGSGGGGS |
| | | GGGGSGGQVQLKQSGPGLVQPSQSLSITCTVSGFS |
| | | LTNYGVHWVRQSPGKGLEWLGVIWSGGNTDYN |
| | | TPFTSRLSINKDNSKSQVFFKMNSLQSNDTAIYYC |
| | | ARALTYYDYEFAYWGQGTLVTVSAASTKGPSVF |
| | | PLAPSSKSTSGGTAALGCLVKDYFPEPVTVSWNS |
| | | GALTSGVHTFPAVLQSSGLYSLSSVVTVPSSSLGT |
| | | QTYICNVNHKPSNTKVDKKVEPKSCDKTHTCPPC |
| | | PAPEAAGGPSVFLFPPKPKDTLMISRTPEVTCVVV |
| | | DVSHEDPEVKFNWYVDGVEVHNAKTKPREEQY |
| | | NSTYRVVSVLTVLHQDWLNGKEYKCKVSNKAL |
| | | GAPIEKTISKAKGQPREPQVYTLPPCRDELTKNQV |
| | | SLWCLVKGFYPSDIAVEWESNGQPENNYKTTPPV |
| | | LDSDGSFFLYSKLTVDKSRWQQGNVFSCSVMHE |
| | | ALHNHYTQKSLSLSPG |
| Her2 binder 1 VH | 130 | EVQLVESGGGLVQPGGSLRLSCAASGFNIKDTYIH |
| | | WVRQAPGKGLEWVARIYPTNGYTRYADSVKGRF |
| | | TISADTSKNTAYLQMNSLRAEDTAVYYCSRWGG |
| | | DGFYAMDYWGQGTLVTVSS |
| Her2 binder 1 VL | 131 | DIQMTQSPSSLSASVGDRVTITCRASQDVNTAVA |
| | | WYQQKPGKAPKLLIYSASFLYSGVPSRFSGSRSGT |
| | | DFTLTISSLQPEDFATYYCQQHYTTPPTFGQGTKV |
| | | EIK |
| Her2 binder 2 VH | 132 | EVQLVESGGGLVQPGGSLRLSCAASGFTFTDYTM |
| | | DWVRQAPGKGLEWVADVNPNSGGSIYNQRFKG |
| | | RFTLSVDRSKNTLYLQMNSLRAEDTAVYYCARN |
| | | LGPSFYFDYWGQGTLVTVSS |
| Her2 binder 2 VL | 133 | DIQMTQSPSSLSASVGDRVTITCKASQDVSIGVAW |
| | | YQQKPGKAPKLLIYSASYRYTGVPSRFSGSGSGT |
| | | DFTLTISSLQPEDFATYYCQQYYIYPYTFGQGTKV |
| | | EIK |
| LC (P1AJ4173, | 134 | DIQMTQSPSSLSASVGDRVTITCRASQDVNTAVA |
| P1AJ4174) | | WYQQKPGKAPKLLIYSASFLYSGVPSRFSGSRSGT |
| | | DFTLTISSLQPEDFATYYCQQHYTTPPTFGQGTKV |
| | | EIKRTVAAPSVFIFPPSDEQLKSGTASVVCLLNNF |
| | | YPREAKVQWKVDNALQSGNSQESVTEQDSKDST |
| | | YSLSSTLTLSKADYEKHKVYACEVTHQGLSSPVT |
| | | KSFNRGEC |
| P1AJ4173 knob chain | 135 | QVQLQESGGGLVQAGGSLRLSCAASISISKIDLMG |
| | | WYRQAPGRQRELVARVTNGGDSYYSTSVKGRFT |
| | | ISRDNAKNTLYLQMNSLKPEDTAVYYCYGVPESL |
| | | AAFHWGQGTLVTVSSGGSGGGGSGGGGSGGGGS |
| | | GGGGSGGEVQLVESGGGLVQPGGSLRLSCAASGF |
| | | NIKDTYIHWVRQAPGKGLEWVARIYPTNGYTRY |
| | | ADSVKGRFTISADTSKNTAYLQMNSLRAEDTAVY |
| | | YCSRWGGDGFYAMDYWGQGTLVTVSSASTKGP |
| | | SVFPLAPSSKSTSGGTAALGCLVKDYFPEPVTVS |
| | | WNSGALTSGVHTFPAVLQSSGLYSLSSVVTVPSSS |
| | | LGTQTYICNVNHKPSNTKVDKKVEPKSCDKTHTC |
| | | PPCPAPEAAGGPSVFLFPPKPKDTLMISRTPEVTC |
| | | VVVDVSHEDPEVKFNWYVDGVEVHNAKTKPRE |
| | | EQYNSTYRVVSVLTVLHQDWLNGKEYKCKVSN |
| | | KALGAPIEKTISKAKGQPREPQVYTLPPCRDELTK |
| | | NQVSLWCLVKGFYPSDIAVEWESNGQPENNYKT |
| | | TPPVLDSDGSFFLYSKLTVDKSRWQQGNVFSCSV |
| | | MHEALHNHYTQKSLSLSPG |
| P1AJ4174 knob chain | 136 | QVQLQQSGGGLVQPGGSLTLSCVASGSILEMSRM |
| | | AWYRQAPGKMRELVASITSIGSIVYADSVKGRFT |
| | | LSRDNTKNTVYLQMNSLKPEDTAVYRCNLLGTW |
| | | SDETYWGQGTQVTVSSGGSGGGGSGGGGSGGGG |
| | | SGGGGSGGEVQLVESGGGLVQPGGSLRLSCAASG |
| | | FNIKDTYIHWVRQAPGKGLEWVARIYPTNGYTRY |
| | | ADSVKGRFTISADTSKNTAYLQMNSLRAEDTAVY |
| | | YCSRWGGDGFYAMDYWGQGTLVTVSSASTKGP |
| | | SVFPLAPSSKSTSGGTAALGCLVKDYFPEPVTVS |
| | | WNSGALTSGVHTFPAVLQSSGLYSLSSVVTVPSSS |
| | | LGTQTYICNVNHKPSNTKVDKKVEPKSCDKTHTC |
| | | PPCPAPEAAGGPSVFLFPPKPKDTLMISRTPEVTC |
| | | VVVDVSHEDPEVKFNWYVDGVEVHNAKTKPRE |
| | | EQYNSTYRVVSVLTVLHQDWLNGKEYKCKVSN |
| | | KALGAPIEKTISKAKGQPREPQVYTLPPCRDELTK |
| | | NQVSLWCLVKGFYPSDIAVEWESNGQPENNYKT |
| | | TPPVLDSDGSFFLYSKLTVDKSRWQQGNVFSCSV |
| | | MHEALHNHYTQKSLSLSPG |
| P1AJ4175 LC | 137 | QVQLQESGGGLVQAGGSLRLSCAASISISKIDLMG |
| | | WYRQAPGRQRELVARVTNGGDSYYSTSVKGRFT |
| | | ISRDNAKNTLYLQMNSLKPEDTAVYYCYGVPESL |
| | | AAFHWGQGTLVTVSSGGSGGGGSGGGGSGGGGS |
| | | GGGGSGGDIQMTQSPSSLSASVGDRVTITCKASQ |
| | | DVSIGVAWYQQKPGKAPKLLIYSASYRYTGVPSR |
| | | FSGSGSGTDFTLTISSLQPEDFATYYCQQYYIYPYT |
| | | FGQGTKVEIKRTVAAPSVFIFPPSDEQLKSGTASV |
| | | VCLLNNFYPREAKVQWKVDNALQSGNSQESVTE |
| | | QDSKDSTYSLSSTLTLSKADYEKHKVYACEVTHQ |
| | | GLSSPVTKSFNRGEC |
| knob chain (P1AJ4175, | 138 | EVQLVESGGGLVQPGGSLRLSCAASGFTFTDYTM |
| P1AJ4176) | | DWVRQAPGKGLEWVADVNPNSGGSIYNQRFKG |
| | | RFTLSVDRSKNTLYLQMNSLRAEDTAVYYCARN |
| | | LGPSFYFDYWGQGTLVTVSSASTKGPSVFPLAPSS |
| | | KSTSGGTAALGCLVKDYFPEPVTVSWNSGALTSG |
| | | VHTFPAVLQSSGLYSLSSVVTVPSSSLGTQTYICN |
| | | VNHKPSNTKVDKKVEPKSCDKTHTCPPCPAPEAA |
| | | GGPSVFLFPPKPKDTLMISRTPEVTCVVVDVSHED |
| | | PEVKFNWYVDGVEVHNAKTKPREEQYNSTYRVV |
| | | SVLTVLHQDWLNGKEYKCKVSNKALGAPIEKTIS |
| | | KAKGQPREPQVYTLPPCRDELTKNQVSLWCLVK |
| | | GFYPSDIAVEWESNGQPENNYKTTPPVLDSDGSFF |
| | | LYSKLTVDKSRWQQGNVFSCSVMHEALHNHYTQ |
| | | KSLSLSPG |
| P1AJ4176 LC | 139 | QVQLQQSGGGLVQPGGSLTLSCVASGSILEMSRM |
| | | AWYRQAPGKMRELVASITSIGSIVYADSVKGRFT |
| | | LSRDNTKNTVYLQMNSLKPEDTAVYRCNLLGTW |
| | | SDETYWGQGTQVTVSSGGSGGGGSGGGGSGGGG |
| | | SGGGGSGGDIQMTQSPSSLSASVGDRVTITCKASQ |
| | | DVSIGVAWYQQKPGKAPKLLIYSASYRYTGVPSR |
| | | FSGSGSGTDFTLTISSLQPEDFATYYCQQYYIYPYT |
| | | FGQGTKVEIKRTVAAPSVFIFPPSDEQLKSGTASV |
| | | VCLLNNFYPREAKVQWKVDNALQSGNSQESVTE |
| | | QDSKDSTYSLSSTLTLSKADYEKHKVYACEVTHQ |
| | | GLSSPVTKSFNRGEC |
| Her3 binder 1 VH | 140 | QVQLVQSGAEVKKPGASVKVSCKASGYTFRSSYI |
| | | SWVRQAPGQGLEWMGWIYAGTGSPSYNQKLQG |
| | | RVTMTTDTSTSTAYMELRSLRSDDTAVYYCARH |
| | | RDYYSNSLTYWGQGTLVTVSS |
| Her3 binder 1 VL | 141 | DIVMTQSPDSLAVSLGERATINCKSSQSVLNSGNQ |
| | | KNYLTWYQQKPGQPPKLLIYWASTRESGVPDRFS |
| | | GSGSGTDFTLTISSLQAEDVAVYYCQSDYSYPYTF |
| | | GQGTKLEIK |
| Her3 binder 2 VH | 142 | QVQLQQSGAELAKPGASVKMSCKTSGYTLTDYW |
| | | IHWVKQRPGQGLEWIGYINPYTGYTESNQKFKDK |
| | | ATLTADKSSNTAYIQLSSLTSEDSAVYYCARPYY |
| | | YGDYWGQGTTLTVSS |
| Her3 binder 2 VL | 143 | QIVLTQSPAIMSASPGERVTITCSASSSVSYMHWF |
| | | QQKPGTSPKLLIYSTSNLASGVPARFSGSGSGTSY |
| | | SLTISRMEAEDAATYYCQQRSSYPFTFGSGTKLEI |
| | | K |
| LC (P1AJ4177, | 144 | DIVMTQSPDSLAVSLGERATINCKSSQSVLNSGNQ |
| P1AJ4178) | | KNYLTWYQQKPGQPPKLLIYWASTRESGVPDRFS |
| | | GSGSGTDFTLTISSLQAEDVAVYYCQSDYSYPYTF |
| | | GQGTKLEIKRTVAAPSVFIFPPSDEQLKSGTASVV |
| | | CLLNNFYPREAKVQWKVDNALQSGNSQESVTEQ |
| | | DSKDSTYSLSSTLTLSKADYEKHKVYACEVTHQG |
| | | LSSPVTKSFNRGEC |
| P1AJ4177 knob chain | 145 | QVQLQESGGGLVQAGGSLRLSCAASISISKIDLMG |
| | | WYRQAPGRQRELVARVTNGGDSYYSTSVKGRFT |
| | | ISRDNAKNTLYLQMNSLKPEDTAVYYCYGVPESL |
| | | AAFHWGQGTLVTVSSGGSGGGGSGGGGSGGGGS |
| | | GGGGSGGQVQLVQSGAEVKKPGASVKVSCKASG |
| | | YTFRSSYISWVRQAPGQGLEWMGWIYAGTGSPS |
| | | YNQKLQGRVTMTTDTSTSTAYMELRSLRSDDTA |
| | | VYYCARHRDYYSNSLTYWGQGTLVTVSSASTKG |
| | | PSVFPLAPSSKSTSGGTAALGCLVKDYFPEPVTVS |
| | | WNSGALTSGVHTFPAVLQSSGLYSLSSVVTVPSSS |
| | | LGTQTYICNVNHKPSNTKVDKKVEPKSCDKTHTC |
| | | PPCPAPEAAGGPSVFLFPPKPKDTLMISRTPEVTC |
| | | VVVDVSHEDPEVKFNWYVDGVEVHNAKTKPRE |
| | | EQYNSTYRVVSVLTVLHQDWLNGKEYKCKVSN |
| | | KALGAPIEKTISKAKGQPREPQVYTLPPCRDELTK |
| | | NQVSLWCLVKGFYPSDIAVEWESNGQPENNYKT |
| | | TPPVLDSDGSFFLYSKLTVDKSRWQQGNVFSCSV |
| | | MHEALHNHYTQKSLSLSPG |
| P1AJ4178 knob chain | 146 | QVQLQQSGGGLVQPGGSLTLSCVASGSILEMSRM |
| | | AWYRQAPGKMRELVASITSIGSIVYADSVKGRFT |
| | | LSRDNTKNTVYLQMNSLKPEDTAVYRCNLLGTW |
| | | SDETYWGQGTQVTVSSGGSGGGGGGGGSGGGG |
| | | SGGGGSGGQVQLVQSGAEVKKPGASVKVSCKAS |
| | | GYTFRSSYISWVRQAPGQGLEWMGWIYAGTGSP |
| | | SYNQKLQGRVTMTTDTSTSTAYMELRSLRSDDT |
| | | AVYYCARHRDYYSNSLTYWGQGTLVTVSSASTK |
| | | GPSVFPLAPSSKSTSGGTAALGCLVKDYFPEPVTV |
| | | SWNSGALTSGVHTFPAVLQSSGLYSLSSVVTVPSS |
| | | SLGTQTYICNVNHKPSNTKVDKKVEPKSCDKTHT |
| | | CPPCPAPEAAGGPSVFLFPPKPKDTLMISRTPEVTC |
| | | VVVDVSHEDPEVKFNWYVDGVEVHNAKTKPRE |
| | | EQYNSTYRVVSVLTVLHQDWLNGKEYKCKVSN |
| | | KALGAPIEKTISKAKGQPREPQVYTLPPCRDELTK |
| | | NQVSLWCLVKGFYPSDIAVEWESNGQPENNYKT |
| | | TPPVLDSDGSFFLYSKLTVDKSRWQQGNVFSCSV |
| | | MHEALHNHYTQKSLSLSPG |
| P1AJ4179 LC | 147 | QVQLQESGGGLVQAGGSLRLSCAASISISKIDLMG |
| | | WYRQAPGRQRELVARVTNGGDSYYSTSVKGRFT |
| | | ISRDNAKNTLYLQMNSLKPEDTAVYYCYGVPESL |
| | | AAFHWGQGTLVTVSSGGSGGGGSGGGGSGGGGS |
| | | GGGGSGGQIVLTQSPAIMSASPGERVTITCSASSS |
| | | VSYMHWFQQKPGTSPKLLIYSTSNLASGVPARFS |
| | | GSGSGTSYSLTISRMEAEDAATYYCQQRSSYPFTF |
| | | GSGTKLEIKRTVAAPSVFIFPPSDEQLKSGTASVV |
| | | CLLNNFYPREAKVQWKVDNALQSGNSQESVTEQ |
| | | DSKDSTYSLSSTLTLSKADYEKHKVYACEVTHQG |
| | | LSSPVTKSFNRGEC |
| knob chain (P1AJ4179, | 148 | QVQLQQSGAELAKPGASVKMSCKTSGYTLTDYW |
| P1AJ4180) | | IHWVKQRPGQGLEWIGYINPYTGYTESNQKFKDK |
| | | ATLTADKSSNTAYIQLSSLTSEDSAVYYCARPYY |
| | | YGDYWGQGTTLTVSSASTKGPSVFPLAPSSKSTS |
| | | GGTAALGCLVKDYFPEPVTVSWNSGALTSGVHT |
| | | FPAVLQSSGLYSLSSVVTVPSSSLGTQTYICNVNH |
| | | KPSNTKVDKKVEPKSCDKTHTCPPCPAPEAAGGP |
| | | SVFLFPPKPKDTLMISRTPEVTCVVVDVSHEDPEV |
| | | KFNWYVDGVEVHNAKTKPREEQYNSTYRVVSVL |
| | | TVLHQDWLNGKEYKCKVSNKALGAPIEKTISKA |
| | | KGQPREPQVYTLPPCRDELTKNQVSLWCLVKGF |
| | | YPSDIAVEWESNGQPENNYKTTPPVLDSDGSFFL |
| | | YSKLTVDKSRWQQGNVFSCSVMHEALHNHYTQ |
| | | KSLSLSPG |
| P1AJ4180 LC | 149 | QVQLQQSGGGLVQPGGSLTLSCVASGSILEMSRM |
| | | AWYRQAPGKMRELVASITSIGSIVYADSVKGRFT |
| | | LSRDNTKNTVYLQMNSLKPEDTAVYRCNLLGTW |
| | | SDETYWGQGTQVTVSSGGSGGGGSGGGGSGGGG |
| | | SGGGGSGGQIVLTQSPAIMSASPGERVTITCSASSS |
| | | VSYMHWFQQKPGTSPKLLIYSTSNLASGVPARFS |
| | | GSGSGTSYSLTISRMEAEDAATYYCQQRSSYPFTF |
| | | GSGTKLEIKRTVAAPSVFIFPPSDEQLKSGTASVV |
| | | CLLNNFYPREAKVQWKVDNALQSGNSQESVTEQ |
| | | DSKDSTYSLSSTLTLSKADYEKHKVYACEVTHQG |
| | | LSSPVTKSFNRGEC |
| LAG3 binder 1 VH | 150 | EVQLLESGGGLVQPGGSLRLSCAASGFIFDDYTM |
| | | NWVRQAPGKGLEWVAVISWDGGGTYYTDSVKG |
| | | RFTISRDDFKNTLYLQMNSLRAEDTAVYYCAKGL |
| | | TDTTLYGSDYWGQGTLVTVSS |
| LAG3 binder 1 VL | 151 | DIQMTQSPSSLSASVGDRVTITCRASQSISSYLNW |
| | | YQQKPGKAPKLLIYAASTLQSGVPSRFSGSGSGTD |
| | | FTLTISSLQPEDFATYYCQQTYSSPLTFGGGTKVEI |
| | | K |
| LAG3 binder 2 VH | 152 | EVQLVESGGGLVQPGGSLRLACAASGFTFSDYA |
| | | MSWVRQAPGKGLEWVSGIDNSGYYTYYTDSVK |
| | | GRFTISRDDVKNTLYLQMNSLRAEDTAVYLCTKT |
| | | HSGLIVNDAFDIWGQGTMVTVSS |
| LAG3 binder 2 VL | 153 | DIQLTQSPSSLSASVGDRVTITCRASQSISSYLNWY |
| | | QQKPGKAPKLLIYDASSLESGVPSRFSGSGSGTDA |
| | | TLTISSLQPEDFATYYCQQSYSTPLTFGGGTKVEIK |
| P1AJ5654 LC | 154 | QVQLQQSGGGLVQPGGSLTLSCVASGSILEMSRM |
| | | AWYRQAPGKMRELVASITSIGSIVYADSVKGRFT |
| | | LSRDNTKNTVYLQMNSLKPEDTAVYRCNLLGTW |
| | | SDETYWGQGTQVTVSSGGSGGGGSGGGGSGGGG |
| | | SGGGGSGGDIQMTQSPSSLSASVGDRVTITCRASQ |
| | | SISSYLNWYQQKPGKAPKLLIYAASTLQSGVPSRF |
| | | SGSGSGTDFTLTISSLQPEDFATYYCQQTYSSPLTF |
| | | GGGTKVEIKRTVAAPSVFIFPPSDEQLKSGTASVV |
| | | CLLNNFYPREAKVQWKVDNALQSGNSQESVTEQ |
| | | DSKDSTYSLSSTLTLSKADYEKHKVYACEVTHQG |
| | | LSSPVTKSFNRGEC |
| knob chain (P1AJ5654, | 155 | EVQLLESGGGLVQPGGSLRLSCAASGFIFDDYTM |
| P1AJ5655) | | NWVRQAPGKGLEWVAVISWDGGGTYYTDSVKG |
| | | RFTISRDDFKNTLYLQMNSLRAEDTAVYYCAKGL |
| | | TDTTLYGSDYWGQGTLVTVSSASTKGPSVFPLAP |
| | | SSKSTSGGTAALGCLVKDYFPEPVTVSWNSGALT |
| | | SGVHTFPAVLQSSGLYSLSSVVTVPSSSLGTQTYI |
| | | CNVNHKPSNTKVDKKVEPKSCDKTHTCPPCPAPE |
| | | AAGGPSVFLFPPKPKDTLMISRTPEVTCVVVDVSH |
| | | EDPEVKFNWYVDGVEVHNAKTKPREEQYNSTYR |
| | | VVSVLTVLHQDWLNGKEYKCKVSNKALGAPIEK |
| | | TISKAKGQPREPQVYTLPPCRDELTKNQVSLWCL |
| | | VKGFYPSDIAVEWESNGQPENNYKTTPPVLDSDG |
| | | SFFLYSKLTVDKSRWQQGNVFSCSVMHEALHNH |
| | | YTQKSLSLSPG |
| P1AJ5655 LC | 156 | QVQLQESGGGLVQAGGSLRLSCAASISISKIDLMG |
| | | WYRQAPGRQRELVARVTNGGDSYYSTSVKGRFT |
| | | ISRDNAKNTLYLQMNSLKPEDTAVYYCYGVPESL |
| | | AAFHWGQGTLVTVSSGGSGGGGSGGGGSGGGGS |
| | | GGGGSGGDIQMTQSPSSLSASVGDRVTITCRASQS |
| | | ISSYLNWYQQKPGKAPKLLIYAASTLQSGVPSRFS |
| | | GSGSGTDFTLTISSLQPEDFATYYCQQTYSSPLTFG |
| | | GGTKVEIKRTVAAPSVFIFPPSDEQLKSGTASVVC |
| | | LLNNFYPREAKVQWKVDNALQSGNSQESVTEQD |
| | | SKDSTYSLSSTLTLSKADYEKHKVYACEVTHQGL |
| | | SSPVTKSFNRGEC |
| P1AJ5656 LC | 157 | QVQLQQSGGGLVQPGGSLTLSCVASGSILEMSRM |
| | | AWYRQAPGKMRELVASITSIGSIVYADSVKGRFT |
| | | LSRDNTKNTVYLQMNSLKPEDTAVYRCNLLGTW |
| | | SDETYWGQGTQVTVSSGGSGGGGSGGGGSGGGG |
| | | SGGGGSGGDIQLTQSPSSLSASVGDRVTITCRASQ |
| | | SISSYLNWYQQKPGKAPKLLIYDASSLESGVPSRF |
| | | SGSGSGTDATLTISSLQPEDFATYYCQQSYSTPLTF |
| | | GGGTKVEIKRTVAAPSVFIFPPSDEQLKSGTASVV |
| | | CLLNNFYPREAKVQWKVDNALQSGNSQESVTEQ |
| | | DSKDSTYSLSSTLTLSKADYEKHKVYACEVTHQG |
| | | LSSPVTKSFNRGEC |
| knob chain (P1AJ5656, | 158 | EVQLVESGGGLVQPGGSLRLACAASGFTFSDYA |
| P1AJ5657) | | MSWVRQAPGKGLEWVSGIDNSGYYTYYTDSVK |
| | | GRFTISRDDVKNTLYLQMNSLRAEDTAVYLCTKT |
| | | HSGLIVNDAFDIWGQGTMVTVSSASTKGPSVFPL |
| | | APSSKSTSGGTAALGCLVKDYFPEPVTVSWNSGA |
| | | LTSGVHTFPAVLQSSGLYSLSSVVTVPSSSLGTQT |
| | | YICNVNHKPSNTKVDKKVEPKSCDKTHTCPPCPA |
| | | PEAAGGPSVFLFPPKPKDTLMISRTPEVTCVVVDV |
| | | SHEDPEVKFNWYVDGVEVHNAKTKPREEQYNST |
| | | YRVVSVLTVLHQDWLNGKEYKCKVSNKALGAPI |
| | | EKTISKAKGQPREPQVYTLPPCRDELTKNQVSLW |
| | | CLVKGFYPSDIAVEWESNGQPENNYKTTPPVLDS |
| | | DGSFFLYSKLTVDKSRWQQGNVFSCSVMHEALH |
| | | NHYTQKSLSLSPG |
| P1AJ5657 LC | 159 | QVQLQESGGGLVQAGGSLRLSCAASISISKIDLMG |
| | | WYRQAPGRQRELVARVTNGGDSYYSTSVKGRFT |
| | | ISRDNAKNTLYLQMNSLKPEDTAVYYCYGVPESL |
| | | AAFHWGQGTLVTVSSGGSGGGGSGGGGSGGGGS |
| | | GGGGSGGDIQLTQSPSSLSASVGDRVTITCRASQSI |
| | | SSYLNWYQQKPGKAPKLLIYDASSLESGVPSRFS |
| | | GSGSGTDATLTISSLQPEDFATYYCQQSYSTPLTF |
| | | GGGTKVEIKRTVAAPSVFIFPPSDEQLKSGTASVV |
| | | CLLNNFYPREAKVQWKVDNALQSGNSQESVTEQ |
| | | DSKDSTYSLSSTLTLSKADYEKHKVYACEVTHQG |
| | | LSSPVTKSFNRGEC |
| LC (P1AJ5658, | 160 | DIQMTQSPSSLSASVGDRVTITCRASQSISSYLNW |
| P1AJ5659) | | YQQKPGKAPKLLIYAASTLQSGVPSRFSGSGSGTD |
| | | FTLTISSLQPEDFATYYCQQTYSSPLTFGGGTKVEI |
| | | KRTVAAPSVFIFPPSDEQLKSGTASVVCLLNNFYP |
| | | REAKVQWKVDNALQSGNSQESVTEQDSKDSTYS |
| | | LSSTLTLSKADYEKHKVYACEVTHQGLSSPVTKS |
| | | FNRGEC |
| P1AJ5658 knob chain | 161 | QVQLQQSGGGLVQPGGSLTLSCVASGSILEMSRM |
| | | AWYRQAPGKMRELVASITSIGSIVYADSVKGRFT |
| | | LSRDNTKNTVYLQMNSLKPEDTAVYRCNLLGTW |
| | | SDETYWGQGTQVTVSSGGSGGGGSGGGGSGGGG |
| | | SGGGGSGGEVQLLESGGGLVQPGGSLRLSCAASG |
| | | FIFDDYTMNWVRQAPGKGLEWVAVISWDGGGT |
| | | YYTDSVKGRFTISRDDFKNTLYLQMNSLRAEDTA |
| | | VYYCAKGLTDTTLYGSDYWGQGTLVTVSSASTK |
| | | GPSVFPLAPSSKSTSGGTAALGCLVKDYFPEPVTV |
| | | SWNSGALTSGVHTFPAVLQSSGLYSLSSVVTVPSS |
| | | SLGTQTYICNVNHKPSNTKVDKKVEPKSCDKTHT |
| | | CPPCPAPEAAGGPSVFLFPPKPKDTLMISRTPEVTC |
| | | VVVDVSHEDPEVKFNWYVDGVEVHNAKTKPRE |
| | | EQYNSTYRVVSVLTVLHQDWLNGKEYKCKVSN |
| | | KALGAPIEKTISKAKGQPREPQVYTLPPCRDELTK |
| | | NQVSLWCLVKGFYPSDIAVEWESNGQPENNYKT |
| | | TPPVLDSDGSFFLYSKLTVDKSRWQQGNVFSCSV |
| | | MHEALHNHYTQKSLSLSPG |
| P1AJ5659 knob chain | 162 | QVQLQESGGGLVQAGGSLRLSCAASISISKIDLMG |
| | | WYRQAPGRQRELVARVTNGGDSYYSTSVKGRFT |
| | | ISRDNAKNTLYLQMNSLKPEDTAVYYCYGVPESL |
| | | AAFHWGQGTLVTVSSGGSGGGGSGGGGSGGGGS |
| | | GGGGSGGEVQLLESGGGLVQPGGSLRLSCAASGF |
| | | IFDDYTMNWVRQAPGKGLEWVAVISWDGGGTY |
| | | YTDSVKGRFTISRDDFKNTLYLQMNSLRAEDTAV |
| | | YYCAKGLTDTTLYGSDYWGQGTLVTVSSASTKG |
| | | PSVFPLAPSSKSTSGGTAALGCLVKDYFPEPVTVS |
| | | WNSGALTSGVHTFPAVLQSSGLYSLSSVVTVPSSS |
| | | LGTQTYICNVNHKPSNTKVDKKVEPKSCDKTHTC |
| | | PPCPAPEAAGGPSVFLFPPKPKDTLMISRTPEVTC |
| | | VVVDVSHEDPEVKFNWYVDGVEVHNAKTKPRE |
| | | EQYNSTYRVVSVLTVLHQDWLNGKEYKCKVSN |
| | | KALGAPIEKTISKAKGQPREPQVYTLPPCRDELTK |
| | | NQVSLWCLVKGFYPSDIAVEWESNGQPENNYKT |
| | | TPPVLDSDGSFFLYSKLTVDKSRWQQGNVFSCSV |
| | | MHEALHNHYTQKSLSLSPG |
| LC (P1AJ5660, | 163 | DIQLTQSPSSLSASVGDRVTITCRASQSISSYLNWY |
| P1AJ5661) | | QQKPGKAPKLLIYDASSLESGVPSRFSGSGSGTDA |
| | | TLTISSLQPEDFATYYCQQSYSTPLTFGGGTKVEIK |
| | | RTVAAPSVFIFPPSDEQLKSGTASVVCLLNNFYPR |
| | | EAKVQWKVDNALQSGNSQESVTEQDSKDSTYSL |
| | | SSTLTLSKADYEKHKVYACEVTHQGLSSPVTKSF |
| | | NRGEC |
| P1AJ5660 knob chain | 164 | QVQLQQSGGGLVQPGGSLTLSCVASGSILEMSRM |
| | | AWYRQAPGKMRELVASITSIGSIVYADSVKGRFT |
| | | LSRDNTKNTVYLQMNSLKPEDTAVYRCNLLGTW |
| | | SDETYWGQGTQVTVSSGGSGGGGGGGGSGGGG |
| | | SGGGGSGGEVQLVESGGGLVQPGGSLRLACAAS |
| | | GFTFSDYAMSWVRQAPGKGLEWVSGIDNSGYYT |
| | | YYTDSVKGRFTISRDDVKNTLYLQMNSLRAEDTA |
| | | VYLCTKTHSGLIVNDAFDIWGQGTMVTVSSASTK |
| | | GPSVFPLAPSSKSTSGGTAALGCLVKDYFPEPVTV |
| | | SWNSGALTSGVHTFPAVLQSSGLYSLSSVVTVPSS |
| | | SLGTQTYICNVNHKPSNTKVDKKVEPKSCDKTHT |
| | | CPPCPAPEAAGGPSVFLFPPKPKDTLMISRTPEVTC |
| | | VVVDVSHEDPEVKFNWYVDGVEVHNAKTKPRE |
| | | EQYNSTYRVVSVLTVLHQDWLNGKEYKCKVSN |
| | | KALGAPIEKTISKAKGQPREPQVYTLPPCRDELTK |
| | | NQVSLWCLVKGFYPSDIAVEWESNGQPENNYKT |
| | | TPPVLDSDGSFFLYSKLTVDKSRWQQGNVFSCSV |
| | | MHEALHNHYTQKSLSLSPG |
| P1AJ5661 knob chain | 165 | QVQLQESGGGLVQAGGSLRLSCAASISISKIDLMG |
| | | WYRQAPGRQRELVARVTNGGDSYYSTSVKGRFT |
| | | ISRDNAKNTLYLQMNSLKPEDTAVYYCYGVPESL |
| | | AAFHWGQGTLVTVSSGGSGGGGSGGGGSGGGGS |
| | | GGGGSGGEVQLVESGGGLVQPGGSLRLACAASG |
| | | FTFSDYAMSWVRQAPGKGLEWVSGIDNSGYYTY |
| | | YTDSVKGRFTISRDDVKNTLYLQMNSLRAEDTAV |
| | | YLCTKTHSGLIVNDAFDIWGQGTMVTVSSASTKG |
| | | PSVFPLAPSSKSTSGGTAALGCLVKDYFPEPVTVS |
| | | WNSGALTSGVHTFPAVLQSSGLYSLSSVVTVPSSS |
| | | LGTQTYICNVNHKPSNTKVDKKVEPKSCDKTHTC |
| | | PPCPAPEAAGGPSVFLFPPKPKDTLMISRTPEVTC |
| | | VVVDVSHEDPEVKFNWYVDGVEVHNAKTKPRE |
| | | EQYNSTYRVVSVLTVLHQDWLNGKEYKCKVSN |
| | | KALGAPIEKTISKAKGQPREPQVYTLPPCRDELTK |
| | | NQVSLWCLVKGFYPSDIAVEWESNGQPENNYKT |
| | | TPPVLDSDGSFFLYSKLTVDKSRWQQGNVFSCSV |
| | | MHEALHNHYTQKSLSLSPG |
| sdPD-1 binder 1 | 166 | QVQLVESGGGLVQAGGSLALSCAASGRAFGSYN |
| | | MAWFRQAPGKEREYVAVISGSDSARHYAASAKD |
| | | RFTISRDNAKNTVYLQMNSLRPEDTAVYYCAAD |
| | | GAAFIVYRAADYDYWGQGTQVTVSS |
| sdPD-1 binder 2 | 167 | QVQLVESGGGLVQAGGSLRLSCAASGWTHSRYV |
| | | MGWFRQAPGKEREFVAAISWSGGNTVYADSVKG |
| | | RFTISRDNAKNTVYLQMNSLKPEDTADYYCAARS |
| | | SYADAAYYTQSPQYADWGQGTQVTVSS |
| sdPD-1 binder 3 | 168 | QVQLVESGGGLVQAGGSLRLSCAASGLTLSTYN |
| | | MGWFRQAPGKEREYVAVISGSDSARHYAASAKD |
| | | RFTISRDNAKNTVYLQMNSLKPEDTAIYYCAADA |
| | | AGFIVYGAADYDYWGQGTQVTVSS |
| sdPD-1 binder 4 | 169 | QVQLVESGGGLVQAGGSLRLSCAASGWTHSRYV |
| | | MGWFRQAPGKEREFVAAISWSGGNTVYADSVKG |
| | | RFTISRDNAKNTVYLQMNSLEPDDTADYYCAARS |
| | | SYADAAYYTQSPQYADWGQGTQVTVSS |
| P1AK3046 knob chain | 170 | QVQLQESGGGLVQAGGSLRLSCAASISISKIDLMG |
| | | WYRQAPGRQRELVARVTNGGDSYYSTSVKGRFT |
| | | ISRDNAKNTLYLQMNSLKPEDTAVYYCYGVPESL |
| | | AAFHWGQGTLVTVSSGGSGGGGSGGGGSGGGGS |
| | | GGGGSGGQVQLVESGGGLVQAGGSLRLSCAASG |
| | | WTHSRYVMGWFRQAPGKEREFVAAISWSGGNT |
| | | VYADSVKGRFTISRDNAKNTVYLQMNSLEPDDT |
| | | ADYYCAARSSYADAAYYTQSPQYADWGQGTQV |
| | | TVSSGGSGGGGSGGDKTHTCPPCPAPEAAGGPSV |
| | | FLFPPKPKDTLMISRTPEVTCVVVDVSHEDPEVKF |
| | | NWYVDGVEVHNAKTKPREEQYNSTYRVVSVLT |
| | | VLHQDWLNGKEYKCKVSNKALGAPIEKTISKAK |
| | | GQPREPQVYTLPPCRDELTKNQVSLWCLVKGFYP |
| | | SDIAVEWESNGQPENNYKTTPPVLDSDGSFFLYS |
| | | KLTVDKSRWQQGNVFSCSVMHEALHNHYTQKSL |
| | | SLSPG |
| P1AK3048 knob chain | 171 | QVQLQESGGGLVQAGGSLRLSCAASISISKIDLMG |
| | | WYRQAPGRQRELVARVTNGGDSYYSTSVKGRFT |
| | | ISRDNAKNTLYLQMNSLKPEDTAVYYCYGVPESL |
| | | AAFHWGQGTLVTVSSGGSGGGGSGGGGSGGGGS |
| | | GGGGSGGQVQLVESGGGLVQAGGSLRLSCAASG |
| | | WTHSRYVMGWFRQAPGKEREFVAAISWSGGNT |
| | | VYADSVKGRFTISRDNAKNTVYLQMNSLKPEDT |
| | | ADYYCAARSSYADAAYYTQSPQYADWGQGTQV |
| | | TVSSGGSGGGGSGGDKTHTCPPCPAPEAAGGPSV |
| | | FLFPPKPKDTLMISRTPEVTCVVVDVSHEDPEVKF |
| | | NWYVDGVEVHNAKTKPREEQYNSTYRVVSVLT |
| | | VLHQDWLNGKEYKCKVSNKALGAPIEKTISKAK |
| | | GQPREPQVYTLPPCRDELTKNQVSLWCLVKGFYP |
| | | SDIAVEWESNGQPENNYKTTPPVLDSDGSFFLYS |
| | | KLTVDKSRWQQGNVFSCSVMHEALHNHYTQKSL |
| | | SLSPG |
| P1AK3051 knob chain | 172 | QVQLQESGGGLVQAGGSLRLSCAASISISKIDLMG |
| | | WYRQAPGRQRELVARVTNGGDSYYSTSVKGRFT |
| | | ISRDNAKNTLYLQMNSLKPEDTAVYYCYGVPESL |
| | | AAFHWGQGTLVTVSSGGSGGGGSGGGGSGGGGS |
| | | GGGGSGGQVQLVESGGGLVQAGGSLALSCAASG |
| | | RAFGSYNMAWFRQAPGKEREYVAVISGSDSARH |
| | | YAASAKDRFTISRDNAKNTVYLQMNSLRPEDTA |
| | | VYYCAADGAAFIVYRAADYDYWGQGTQVTVSS |
| | | GGSGGGGSGGDKTHTCPPCPAPEAAGGPSVFLFP |
| | | PKPKDTLMISRTPEVTCVVVDVSHEDPEVKFNWY |
| | | VDGVEVHNAKTKPREEQYNSTYRVVSVLTVLHQ |
| | | DWLNGKEYKCKVSNKALGAPIEKTISKAKGQPRE |
| | | PQVYTLPPCRDELTKNQVSLWCLVKGFYPSDIAV |
| | | EWESNGQPENNYKTTPPVLDSDGSFFLYSKLTVD |
| | | KSRWQQGNVFSCSVMHEALHNHYTQKSLSLSPG |
| P1AK3052 knob chain | 173 | QVQLQQSGGGLVQPGGSLTLSCVASGSILEMSRM |
| | | AWYRQAPGKMRELVASITSIGSIVYADSVKGRFT |
| | | LSRDNTKNTVYLQMNSLKPEDTAVYRCNLLGTW |
| | | SDETYWGQGTQVTVSSGGSGGGGSGGGGSGGGG |
| | | SGGGGSGGQVQLVESGGGLVQAGGSLALSCAAS |
| | | GRAFGSYNMAWFRQAPGKEREYVAVISGSDSAR |
| | | HYAASAKDRFTISRDNAKNTVYLQMNSLRPEDT |
| | | AVYYCAADGAAFIVYRAADYDYWGQGTQVTVS |
| | | SGGSGGGGSGGDKTHTCPPCPAPEAAGGPSVFLF |
| | | PPKPKDTLMISRTPEVTCVVVDVSHEDPEVKFNW |
| | | YVDGVEVHNAKTKPREEQYNSTYRVVSVLTVLH |
| | | QDWLNGKEYKCKVSNKALGAPIEKTISKAKGQP |
| | | REPQVYTLPPCRDELTKNQVSLWCLVKGFYPSDI |
| | | AVEWESNGQPENNYKTTPPVLDSDGSFFLYSKLT |
| | | VDKSRWQQGNVFSCSVMHEALHNHYTQKSLSLS |
| | | PG |
| P1AK3053 knob chain | 174 | QVQLQESGGGLVQAGGSLRLSCAASISISKIDLMG |
| | | WYRQAPGRQRELVARVTNGGDSYYSTSVKGRFT |
| | | ISRDNAKNTLYLQMNSLKPEDTAVYYCYGVPESL |
| | | AAFHWGQGTLVTVSSGGSGGGGSGGGGSGGGGS |
| | | GGGGSGGQVQLVESGGGLVQAGGSLRLSCAASG |
| | | LTLSTYNMGWFRQAPGKEREYVAVISGSDSARH |
| | | YAASAKDRFTISRDNAKNTVYLQMNSLKPEDTAI |
| | | YYCAADAAGFIVYGAADYDYWGQGTQVTVSSG |
| | | GSGGGGSGGDKTHTCPPCPAPEAAGGPSVFLFPP |
| | | KPKDTLMISRTPEVTCVVVDVSHEDPEVKFNWY |
| | | VDGVEVHNAKTKPREEQYNSTYRVVSVLTVLHQ |
| | | DWLNGKEYKCKVSNKALGAPIEKTISKAKGQPRE |
| | | PQVYTLPPCRDELTKNQVSLWCLVKGFYPSDIAV |
| | | EWESNGQPENNYKTTPPVLDSDGSFFLYSKLTVD |
| | | KSRWQQGNVFSCSVMHEALHNHYTQKSLSLSPG |
| P1AK3054 knob chain | 175 | QVQLQQSGGGLVQPGGSLTLSCVASGSILEMSRM |
| | | AWYRQAPGKMRELVASITSIGSIVYADSVKGRFT |
| | | LSRDNTKNTVYLQMNSLKPEDTAVYRCNLLGTW |
| | | SDETYWGQGTQVTVSSGGSGGGGSGGGGSGGGG |
| | | SGGGGSGGQVQLVESGGGLVQAGGSLRLSCAAS |
| | | GLTLSTYNMGWFRQAPGKEREYVAVISGSDSAR |
| | | HYAASAKDRFTISRDNAKNTVYLQMNSLKPEDT |
| | | AIYYCAADAAGFIVYGAADYDYWGQGTQVTVSS |
| | | GGSGGGGSGGDKTHTCPPCPAPEAAGGPSVFLFP |
| | | PKPKDTLMISRTPEVTCVVVDVSHEDPEVKFNWY |
| | | VDGVEVHNAKTKPREEQYNSTYRVVSVLTVLHQ |
| | | DWLNGKEYKCKVSNKALGAPIEKTISKAKGQPRE |
| | | PQVYTLPPCRDELTKNQVSLWCLVKGFYPSDIAV |
| | | EWESNGQPENNYKTTPPVLDSDGSFFLYSKLTVD |
| | | KSRWQQGNVFSCSVMHEALHNHYTQKSLSLSPG |
| P1AI0063 knob chain | 176 | QVQLQQSGGGLVQPGGSLTLSCVASGSILEMSRM |
| | | AWYRQAPGKMRELVASITSIGSIVYADSVKGRFT |
| | | LSRDNTKNTVYLQMNSLKPEDTAVYRCNLLGTW |
| | | SDETYWGQGTQVTVSSGGSGGGGSGGGGSGGGG |
| | | SGGGGSGGQVQLVESGGGLVQAGGSLRLSCAAS |
| | | GWTHSRYVMGWFRQAPGKEREFVAAISWSGGN |
| | | TVYADSVKGRFTISRDNAKNTVYLQMNSLKPEDT |
| | | ADYYCAARSSYADAAYYTQSPQYADWGQGTQV |
| | | TVSSGGSGGGGSGGDKTHTCPPCPAPEAAGGPSV |
| | | FLFPPKPKDTLMISRTPEVTCVVVDVSHEDPEVKF |
| | | NWYVDGVEVHNAKTKPREEQYNSTYRVVSVLT |
| | | VLHQDWLNGKEYKCKVSNKALGAPIEKTISKAK |
| | | GQPREPQVYTLPPCRDELTKNQVSLWCLVKGFYP |
| | | SDIAVEWESNGQPENNYKTTPPVLDSDGSFFLYS |
| | | KLTVDKSRWQQGNVFSCSVMHEALHNHYTQKSL |
| | | SLSPG |
| IL-7Rα_1 VHH | 177 | QVQLVESGGGLVQPGGSLRLSCAASGFTFSQRIM |
| | | SWYRQAPGKERELVASISPFGDGTRYADSVKGRF |
| | | TISRDNARNQLYLQMNTLKPEDTAVYYCNKRNL |
| | | EISDDWGQGTQVTVSS |
| IL-7Rα_2 VHH | 178 | QVQLVESGGGLVQPGGSLRLSCEVSGIRFSLRNM |
| | | HWYRQASGRQREWVATITAGGVTGYRDSVKGR |
| | | FTISRDNAKNTVDLQMNGLQLEDTAVYYCNHGD |
| | | TAYWGQGTQVTVSS |
| IL-7Rα_3 VHH | 179 | QVQLVESGGGSVQPGGSLRLSCATSGSIFSLATMT |
| | | WYRQGPGKTRELVAVITWRGTTTYADSVKGRFTI |
| | | SRDNAKNTMDLQMNGLKPEDTAVYYCNVKWG |
| | | GGLNREPFDYWGQGTQVTVSS |
| IL-7Rα_4 VHH | 180 | QVQLVESGGGLVQPGGSLRLSCTTSGFTFTTYAM |
| | | KWVRQAPGKGLEWVAFISPAGGITDYADSVKGR |
| | | FTISRDNAKETLYLRMDSLKPEDTAVYYCARGDS |
| | | DTNNNRGQGTQVTVSS |
| IL-7Rα_5 VHH | 181 | QVQLVESGGGLVQPGGSLRLSCAASGFTFSQRIM |
| | | SWYRQAPGKEHELVASISPFGDGTRYADSVKGRF |
| | | TISRDNARNQLYLQMNSLEFEDTAVYYCNKRNL |
| | | DVSDDWGQGTQVTVSS |
| IL-7Rα_6 VHH | 182 | QVQLVESGGGSVQPGGSLRLSCATSGSIFSLATMT |
| | | WYRQGPGKTRELVAVITWRGTTTYADSVKGRFTI |
| | | SRDNAKNTMDLQMNGLKPEDTAVYYCNVKWG |
| | | GGLNRAPFDYWGQGTQVTVSS |
| IL-7Rα_7 VHH | 183 | QVQLVESGGGLVQPGGSLRLSCAASGFTFSNYAM |
| | | SWYRQAPGKERELVASISVFGGGTNYADSVKGRF |
| | | TISRDNAKNTVYLQMNSLKPEDSAVYYCNKRRY |
| | | DDYWGQGTQVTVSS |
| IL-7Rα_8 VHH | 184 | QVQLVESGGGLVQPGGSLRLSCAASGFTISSWSM |
| | | KWVRQAPGKGPEWVSYISPGGGSLDYLDSVKGR |
| | | FTISRDNAKNTVYLQMSNLKSEDTALYWCATGT |
| | | GPTRGQGTQVTVSS |
| IL-7Rα_9 VHH | 185 | QVQLVESGGGLVQPGGSLRLSCAASGFTISTWSM |
| | | KWVRQAPGKGPEWVSYISPGGTSLDYQDSVKGR |
| | | FTISRDNAKNTVYLQMNNLKSEDTALYWCATGT |
| | | GETRGQGTQVTVSS |
| IL-7Rα_10 VHH | 186 | QVQLVESGGGLVQPGGSLRLSCATSGSIFSLATMT |
| | | WYRQAPGKTREFVAAITWRGAATYAESVKGRFT |
| | | ISRDNAKNTMDLQMDGLKPEDTAVYYCNVKWG |
| | | GGLNRAPFDYWGQGTQVTVSS |
| IL-7Rα_11 VHH | 187 | QVQLVESGGGLVQPGGSLRLSCEASGFTISSWSM |
| | | KWVRQAPGKGPEWVSYISPGGGSLDYHHSVKGR |
| | | FTISRDNARNTVYLQMSNLQSEDTALYWCATGT |
| | | GETRGQGTQVTVSS |
| IL-7Rα_12 VHH | 188 | QVQLVESGGGLVQPGGSLRLSCAASGFTISSRSM |
| | | KWVRQAPGKGPEWVSYISPGGGTVDYQDSVKGR |
| | | FTISRDNAKNTVYLQMSNLKSEDTALYWCATGT |
| | | GPTRGRGTQVTVSS |
| IL-7Rα_13 VHH | 189 | QVQLVESGGGLVRPGGSLRLACAASGIRFSLRNM |
| | | HWYRQASGRQREWVATITAGGVTGYRDSVKGR |
| | | FTISRDNAKNTVDLQMNGLQLEDTAVYYCNHGD |
| | | TAYWGQGTQVTVSS |
| IL-7Rα_14 VHH | 190 | QVQLVESGGGLVQPGGSLRLSCAASGFTFSQRIM |
| | | SWYRQAPGKERELVASVSPFGDGTRYADSVKGR |
| | | FTISRDNAKSQLYLQMNNLKFEDTAVYYCNKRN |
| | | LEISDAWGQGTQVTVSS |
| IL-7Rα_15 VHH | 191 | QVQLVESGGGLVQPGGSLRLSCAASGFTISSWSM |
| | | KWVRQAPGKGPEWVSYISPGGGSLDYHHSVKGR |
| | | FTISRDNARNTVYLQMSNLQSEDTALYWCATGT |
| | | GETRGQGTQVTVSS |
| IL-7Rα_16 VHH | 192 | QVQLVESGGGLVQPGGSLRLSCTASGSIFSLATMT |
| | | WYRQAPGKTREFVAAITWRGAATYAESVKGRFT |
| | | ISRDNAKNTMDLQMDGLKPEDTAVYYCNVKWG |
| | | GGLNRAPFDYWGQGTQVTVSS |
| P1AL0544 LC | 193 | QVQLVESGGGLVQPGGSLRLSCAASGFTFSQRIM |
| | | SWYRQAPGKERELVASISPFGDGTRYADSVKGRF |
| | | TISRDNARNQLYLQMNTLKPEDTAVYYCNKRNL |
| | | EISDDWGQGTQVTVSSGGSGGGGSGGGGSGGGG |
| | | SGGGGSGGDIVMTQSPDSLAVSLGERATINCKAS |
| | | ESVDTSDNSFIHWYQQKPGQSPKLLIYRSSTLESG |
| | | VPDRFSGSGSGTDFTLTISSLQAEDVAVYYCQQN |
| | | YDVPWTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVYA |
| | | CEVTHQGLSSPVTKSFNRGEC |
| P1AL0544 - P1AL0556 | 194 | EVQLLESGGGLVQPGGSLRLSCAASGFSFSSYTMS |
| knob chain | | WVRQAPGKGLEWVATISGGGRDIYYPDSVKGRF |
| | | TISRDNSKNTLYLQMNSLRAEDTAVYYCVLLTGR |
| | | VYFALDSWGQGTLVTVSSASTKGPSVFPLAPSSK |
| | | STSGGTAALGCLVKDYFPEPVTVSWNSGALTSGV |
| | | HTFPAVLQSSGLYSLSSVVTVPSSSLGTQTYICNV |
| | | NHKPSNTKVDKKVEPKSCDKTHTCPPCPAPEAAG |
| | | GPSVFLFPPKPKDTLMISRTPEVTCVVVDVSHEDP |
| | | EVKFNWYVDGVEVHNAKTKPREEQYNSTYRVVS |
| | | VLTVLHQDWLNGKEYKCKVSNKALGAPIEKTISK |
| | | AKGQPREPQVYTLPPCRDELTKNQVSLWCLVKG |
| | | FYPSDIAVEWESNGQPENNYKTTPPVLDSDGSFFL |
| | | YSKLTVDKSRWQQGNVFSCSVMHEALHNHYTQ |
| | | KSLSLSPG |
| P1AL0545 LC | 195 | QVQLVESGGGLVQPGGSLRLSCEVSGIRFSLRNM |
| | | HWYRQASGRQREWVATITAGGVTGYRDSVKGR |
| | | FTISRDNAKNTVDLQMNGLQLEDTAVYYCNHGD |
| | | TAYWGQGTQVTVSSGGSGGGGSGGGGSGGGGS |
| | | GGGGSGGDIVMTQSPDSLAVSLGERATINCKASE |
| | | SVDTSDNSFIHWYQQKPGQSPKLLIYRSSTLESGV |
| | | PDRFSGSGSGTDFTLTISSLQAEDVAVYYCQQNY |
| | | DVPWTFGQGTKVEIKRTVAAPSVFIFPPSDEQLKS |
| | | GTASVVCLLNNFYPREAKVQWKVDNALQSGNSQ |
| | | ESVTEQDSKDSTYSLSSTLTLSKADYEKHKVYAC |
| | | EVTHQGLSSPVTKSFNRGEC |
| P1AL0546 LC | 196 | QVQLVESGGGSVQPGGSLRLSCATSGSIFSLATMT |
| | | WYRQGPGKTRELVAVITWRGTTTYADSVKGRFTI |
| | | SRDNAKNTMDLQMNGLKPEDTAVYYCNVKWG |
| | | GGLNREPFDYWGQGTQVTVSSGGSGGGGSGGGG |
| | | SGGGGSGGGGSGGDIVMTQSPDSLAVSLGERATI |
| | | NCKASESVDTSDNSFIHWYQQKPGQSPKLLIYRSS |
| | | TLESGVPDRFSGSGSGTDFTLTISSLQAEDVAVYY |
| | | CQQNYDVPWTFGQGTKVEIKRTVAAPSVFIFPPS |
| | | DEQLKSGTASVVCLLNNFYPREAKVQWKVDNAL |
| | | QSGNSQESVTEQDSKDSTYSLSSTLTLSKADYEK |
| | | HKVYACEVTHQGLSSPVTKSFNRGEC |
| P1AL0547 LC | 197 | QVQLVESGGGLVQPGGSLRLSCAASGFTFSQRIM |
| | | SWYRQAPGKEHELVASISPFGDGTRYADSVKGRF |
| | | TISRDNARNQLYLQMNSLEFEDTAVYYCNKRNL |
| | | DVSDDWGQGTQVTVSSGGSGGGGSGGGGSGGG |
| | | GSGGGGSGGDIVMTQSPDSLAVSLGERATINCKA |
| | | SESVDTSDNSFIHWYQQKPGQSPKLLIYRSSTLES |
| | | GVPDRFSGSGSGTDFTLTISSLQAEDVAVYYCQQ |
| | | NYDVPWTFGQGTKVEIKRTVAAPSVFIFPPSDEQL |
| | | KSGTASVVCLLNNFYPREAKVQWKVDNALQSGN |
| | | SQESVTEQDSKDSTYSLSSTLTLSKADYEKHKVY |
| | | ACEVTHQGLSSPVTKSFNRGEC |
| P1AL0548 LC | 198 | QVQLVESGGGSVQPGGSLRLSCATSGSIFSLATMT |
| | | WYRQGPGKTRELVAVITWRGTTTYADSVKGRFTI |
| | | SRDNAKNTMDLQMNGLKPEDTAVYYCNVKWG |
| | | GGLNRAPFDYWGQGTQVTVSSGGSGGGGSGGG |
| | | GSGGGGSGGGGSGGDIVMTQSPDSLAVSLGERAT |
| | | INCKASESVDTSDNSFIHWYQQKPGQSPKLLIYRS |
| | | STLESGVPDRFSGSGSGTDFTLTISSLQAEDVAVY |
| | | YCQQNYDVPWTFGQGTKVEIKRTVAAPSVFIFPP |
| | | SDEQLKSGTASVVCLLNNFYPREAKVQWKVDNA |
| | | LQSGNSQESVTEQDSKDSTYSLSSTLTLSKADYEK |
| | | HKVYACEVTHQGLSSPVTKSFNRGEC |
| P1AL0549 LC | 199 | QVQLVESGGGLVQPGGSLRLSCAASGFTFSNYAM |
| | | SWYRQAPGKERELVASISVFGGGTNYADSVKGRF |
| | | TISRDNAKNTVYLQMNSLKPEDSAVYYCNKRRY |
| | | DDYWGQGTQVTVSSGGSGGGGSGGGGSGGGGS |
| | | GGGGSGGDIVMTQSPDSLAVSLGERATINCKASE |
| | | SVDTSDNSFIHWYQQKPGQSPKLLIYRSSTLESGV |
| | | PDRFSGSGSGTDFTLTISSLQAEDVAVYYCQQNY |
| | | DVPWTFGQGTKVEIKRTVAAPSVFIFPPSDEQLKS |
| | | GTASVVCLLNNFYPREAKVQWKVDNALQSGNSQ |
| | | ESVTEQDSKDSTYSLSSTLTLSKADYEKHKVYAC |
| | | EVTHQGLSSPVTKSFNRGEC |
| P1AL0550 LC | 200 | QVQLVESGGGLVQPGGSLRLSCATSGSIFSLATMT |
| | | WYRQAPGKTREFVAAITWRGAATYAESVKGRFT |
| | | ISRDNAKNTMDLQMDGLKPEDTAVYYCNVKWG |
| | | GGLNRAPFDYWGQGTQVTVSSGGSGGGGSGGG |
| | | GSGGGGSGGGGSGGDIVMTQSPDSLAVSLGERAT |
| | | INCKASESVDTSDNSFIHWYQQKPGQSPKLLIYRS |
| | | STLESGVPDRFSGSGSGTDFTLTISSLQAEDVAVY |
| | | YCQQNYDVPWTFGQGTKVEIKRTVAAPSVFIFPP |
| | | SDEQLKSGTASVVCLLNNFYPREAKVQWKVDNA |
| | | LQSGNSQESVTEQDSKDSTYSLSSTLTLSKADYEK |
| | | HKVYACEVTHQGLSSPVTKSFNRGEC |
| P1AL0551 LC | 201 | QVQLVESGGGLVQPGGSLRLSCEASGFTISSWSM |
| | | KWVRQAPGKGPEWVSYISPGGGSLDYHHSVKGR |
| | | FTISRDNARNTVYLQMSNLQSEDTALYWCATGT |
| | | GETRGQGTQVTVSSGGSGGGGSGGGGSGGGGSG |
| | | GGGSGGDIVMTQSPDSLAVSLGERATINCKASES |
| | | VDTSDNSFIHWYQQKPGQSPKLLIYRSSTLESGVP |
| | | DRFSGSGSGTDFTLTISSLQAEDVAVYYCQQNYD |
| | | VPWTFGQGTKVEIKRTVAAPSVFIFPPSDEQLKSG |
| | | TASVVCLLNNFYPREAKVQWKVDNALQSGNSQE |
| | | SVTEQDSKDSTYSLSSTLTLSKADYEKHKVYACE |
| | | VTHQGLSSPVTKSFNRGEC |
| P1AL0552 LC | 202 | QVQLVESGGGLVRPGGSLRLACAASGIRFSLRNM |
| | | HWYRQASGRQREWVATITAGGVTGYRDSVKGR |
| | | FTISRDNAKNTVDLQMNGLQLEDTAVYYCNHGD |
| | | TAYWGQGTQVTVSSGGSGGGGSGGGGSGGGGS |
| | | GGGGSGGDIVMTQSPDSLAVSLGERATINCKASE |
| | | SVDTSDNSFIHWYQQKPGQSPKLLIYRSSTLESGV |
| | | PDRFSGSGSGTDFTLTISSLQAEDVAVYYCQQNY |
| | | DVPWTFGQGTKVEIKRTVAAPSVFIFPPSDEQLKS |
| | | GTASVVCLLNNFYPREAKVQWKVDNALQSGNSQ |
| | | ESVTEQDSKDSTYSLSSTLTLSKADYEKHKVYAC |
| | | EVTHQGLSSPVTKSFNRGEC |
| P1AL0553 LC | 203 | QVQLVESGGGLVQPGGSLRLSCAASGFTFSQRIM |
| | | SWYRQAPGKERELVASVSPFGDGTRYADSVKGR |
| | | FTISRDNAKSQLYLQMNNLKFEDTAVYYCNKRN |
| | | LEISDAWGQGTQVTVSSGGSGGGGSGGGGSGGG |
| | | GSGGGGSGGDIVMTQSPDSLAVSLGERATINCKA |
| | | SESVDTSDNSFIHWYQQKPGQSPKLLIYRSSTLES |
| | | GVPDRFSGSGSGTDFTLTISSLQAEDVAVYYCQQ |
| | | NYDVPWTFGQGTKVEIKRTVAAPSVFIFPPSDEQL |
| | | KSGTASVVCLLNNFYPREAKVQWKVDNALQSGN |
| | | SQESVTEQDSKDSTYSLSSTLTLSKADYEKHKVY |
| | | ACEVTHQGLSSPVTKSFNRGEC |
| P1AL0555 LC | 204 | QVQLVESGGGLVQPGGSLRLSCAASGFTISSWSM |
| | | KWVRQAPGKGPEWVSYISPGGGSLDYHHSVKGR |
| | | FTISRDNARNTVYLQMSNLQSEDTALYWCATGT |
| | | GETRGQGTQVTVSSGGSGGGGSGGGGSGGGGSG |
| | | GGGSGGDIVMTQSPDSLAVSLGERATINCKASES |
| | | VDTSDNSFIHWYQQKPGQSPKLLIYRSSTLESGVP |
| | | DRFSGSGSGTDFTLTISSLQAEDVAVYYCQQNYD |
| | | VPWTFGQGTKVEIKRTVAAPSVFIFPPSDEQLKSG |
| | | TASVVCLLNNFYPREAKVQWKVDNALQSGNSQE |
| | | SVTEQDSKDSTYSLSSTLTLSKADYEKHKVYACE |
| | | VTHQGLSSPVTKSFNRGEC |
| P1AL0556 LC | 205 | QVQLVESGGGLVQPGGSLRLSCTASGSIFSLATMT |
| | | WYRQAPGKTREFVAAITWRGAATYAESVKGRFT |
| | | ISRDNAKNTMDLQMDGLKPEDTAVYYCNVKWG |
| | | GGLNRAPFDYWGQGTQVTVSSGGSGGGGSGGG |
| | | GSGGGGSGGGGSGGDIVMTQSPDSLAVSLGERAT |
| | | INCKASESVDTSDNSFIHWYQQKPGQSPKLLIYRS |
| | | STLESGVPDRFSGSGSGTDFTLTISSLQAEDVAVY |
| | | YCQQNYDVPWTFGQGTKVEIKRTVAAPSVFIFPP |
| | | SDEQLKSGTASVVCLLNNFYPREAKVQWKVDNA |
| | | LQSGNSQESVTEQDSKDSTYSLSSTLTLSKADYEK |
| | | HKVYACEVTHQGLSSPVTKSFNRGEC |
| P1AA5355 knob chain | 206 | EVQLLESGGGLVQPGGSLRLSCAASGFTFSSYAM |
| | | SWVRQAPGKGLEWVSAIIGSGASTYYADSVKGRF |
| | | TISRDNSKNTLYLQMNSLRAEDTAVYYCAKGWF |
| | | GGFNYWGQGTLVTVSSASTKGPSVFPLAPSSKST |
| | | SGGTAALGCLVKDYFPEPVTVSWNSGALTSGVH |
| | | TFPAVLQSSGLYSLSSVVTVPSSSLGTQTYICNVN |
| | | HKPSNTKVDKKVEPKSCDKTHTCPPCPAPEAAGG |
| | | PSVFLFPPKPKDTLMISRTPEVTCVVVDVSHEDPE |
| | | VKFNWYVDGVEVHNAKTKPREEQYNSTYRVVS |
| | | VLTVLHQDWLNGKEYKCKVSNKALGAPIEKTISK |
| | | AKGQPREPQVYTLPPCRDELTKNQVSLWCLVKG |
| | | FYPSDIAVEWESNGQPENNYKTTPPVLDSDGSFFL |
| | | YSKLTVDKSRWQQGNVFSCSVMHEALHNHYTQ |
| | | KSLSLSPGGGGGSGGGGSGGGGSAPASSSTKKTQ |
| | | LQLEHLLLDLQMILNGINNYKNPKLTRMLTAKFA |
| | | MPKKATELKHLQCLEEELKPLEEVLNGAQSKNFH |
| | | LRPRDLISNINVIVLELKGSETTFMCEYADETATIV |
| | | EFLNRWITFAQSIISTLT |
| P1AA5355 hole chain | 207 | EVQLLESGGGLVQPGGSLRLSCAASGFTFSSYAM |
| | | SWVRQAPGKGLEWVSAIIGSGASTYYADSVKGRF |
| | | TISRDNSKNTLYLQMNSLRAEDTAVYYCAKGWF |
| | | GGFNYWGQGTLVTVSSASTKGPSVFPLAPSSKST |
| | | SGGTAALGCLVKDYFPEPVTVSWNSGALTSGVH |
| | | TFPAVLQSSGLYSLSSVVTVPSSSLGTQTYICNVN |
| | | HKPSNTKVDKKVEPKSCDKTHTCPPCPAPEAAGG |
| | | PSVFLFPPKPKDTLMISRTPEVTCVVVDVSHEDPE |
| | | VKFNWYVDGVEVHNAKTKPREEQYNSTYRVVS |
| | | VLTVLHQDWLNGKEYKCKVSNKALGAPIEKTISK |
| | | AKGQPREPQVCTLPPSRDELTKNQVSLSCAVKGF |
| | | YPSDIAVEWESNGQPENNYKTTPPVLDSDGSFFL |
| | | VSKLTVDKSRWQQGNVFSCSVMHEALHNHYTQ |
| | | KSLSLSPGK |
| IL-7Rα ECD fused to | 208 | ESGYAQNGDLEDAELDDYSFSCYSQLEVNGSQHS |
| biotinylated Fc knob | | LTCAFEDPDVNITNLEFEICGALVEVKCLNFRKLQ |
| (P1AI1009) | | EIYFIETKKFLLIGKSNICVKVGEKSLTCKKIDLTTI |
| | | VKPEAPFDLSVVYREGANDFVVTFNTSHLQKKY |
| | | VKVLMHDVAYRQEKDENKWTHVNLSSTKLTLL |
| | | QRKLQPAAMYEIKVRSIPDHYFKGFWSEWSPSYY |
| | | FRTPEINNSSGEMDASGSDKTHTCPPCPAPELLGG |
| | | PSVFLFPPKPKDTLMISRTPEVTCVVVDVSHEDPE |
| | | VKFNWYVDGVEVHNAKTKPREEQYNSTYRVVS |
| | | VLTVLHQDWLNGKEYKCKVSNKALPAPIEKTISK |
| | | AKGQPREPQVYTLPPCRDELTKNQVSLWCLVKG |
| | | FYPSDIAVEWESNGQPENNYKTTPPVLDSDGSFFL |
| | | YSKLTVDKSRWQQGNVFSCSVMHEALHNHYTQ |
| | | KSLSLSPGKSGGLNDIFEAQKIEWHE |
| Fc hole (P1AI1009) | 209 | DKTHTCPPCPAPELLGGPSVFLFPPKPKDTLMISRT |
| | | PEVTCVVVDVSHEDPEVKFNWYVDGVEVHNAK |
| | | TKPREEQYNSTYRVVSVLTVLHQDWLNGKEYKC |
| | | KVSNKALPAPIEKTISKAKGQPREPQVCTLPPSRD |
| | | ELTKNQVSLSCAVKGFYPSDIAVEWESNGQPENN |
| | | YKTTPPVLDSDGSFFLVSKLTVDKSRWQQGNVFS |
| | | CSVMHEALHNHYTQKSLSLSPG |
| IL-7Rα_1 heavy chain | 210 | QVQLVESGGGLVQPGGSLRLSCAASGFTFSQRIM |
| (knob chain) | | SWYRQAPGKERELVASISPFGDGTRYADSVKGRF |
| | | TISRDNARNQLYLQMNTLKPEDTAVYYCNKRNL |
| | | EISDDWGQGTQVTVSSGGSGGGGSGGGGSGGGG |
| | | SGGGGSGGDKTHTCPPCPAPEAAGGPSVFLFPPKP |
| | | KDTLMISRTPEVTCVVVDVSHEDPEVKFNWYVD |
| | | GVEVHNAKTKPREEQYNSTYRVVSVLTVLHQDW |
| | | LNGKEYKCKVSNKALGAPIEKTISKAKGQPREPQ |
| | | VYTLPPCRDELTKNQVSLWCLVKGFYPSDIAVEW |
| | | ESNGQPENNYKTTPPVLDSDGSFFLYSKLTVDKS |
| | | RWQQGNVFSCSVMHEALHNHYTQKSLSLSPG |
| IL-7Rα_2 heavy chain | 211 | QVQLVESGGGLVQPGGSLRLSCEVSGIRFSLRNM |
| (knob chain) | | HWYRQASGRQREWVATITAGGVTGYRDSVKGR |
| | | FTISRDNAKNTVDLQMNGLQLEDTAVYYCNHGD |
| | | TAYWGQGTQVTVSSGGSGGGGSGGGGSGGGGS |
| | | GGGGSGGDKTHTCPPCPAPEAAGGPSVFLFPPKP |
| | | KDTLMISRTPEVTCVVVDVSHEDPEVKFNWYVD |
| | | GVEVHNAKTKPREEQYNSTYRVVSVLTVLHQDW |
| | | LNGKEYKCKVSNKALGAPIEKTISKAKGQPREPQ |
| | | VYTLPPCRDELTKNQVSLWCLVKGFYPSDIAVEW |
| | | ESNGQPENNYKTTPPVLDSDGSFFLYSKLTVDKS |
| | | RWQQGNVFSCSVMHEALHNHYTQKSLSLSPG |
| IL-7Rα_3 heavy chain | 212 | QVQLVESGGGSVQPGGSLRLSCATSGSIFSLATMT |
| (knob chain) | | WYRQGPGKTRELVAVITWRGTTTYADSVKGRFTI |
| | | SRDNAKNTMDLQMNGLKPEDTAVYYCNVKWG |
| | | GGLNREPFDYWGQGTQVTVSSGGSGGGGSGGGG |
| | | SGGGGSGGGGSGGDKTHTCPPCPAPEAAGGPSVF |
| | | LFPPKPKDTLMISRTPEVTCVVVDVSHEDPEVKFN |
| | | WYVDGVEVHNAKTKPREEQYNSTYRVVSVLTVL |
| | | HQDWLNGKEYKCKVSNKALGAPIEKTISKAKGQ |
| | | PREPQVYTLPPCRDELTKNQVSLWCLVKGFYPSD |
| | | IAVEWESNGQPENNYKTTPPVLDSDGSFFLYSKLT |
| | | VDKSRWQQGNVFSCSVMHEALHNHYTQKSLSLS |
| | | PG |
| IL-7Rα_4 heavy chain | 213 | QVQLVESGGGLVQPGGSLRLSCTTSGFTFTTYAM |
| (knob chain) | | KWVRQAPGKGLEWVAFISPAGGITDYADSVKGR |
| | | FTISRDNAKETLYLRMDSLKPEDTAVYYCARGDS |
| | | DTNNNRGQGTQVTVSSGGSGGGGSGGGGSGGGG |
| | | SGGGGSGGDKTHTCPPCPAPEAAGGPSVFLFPPKP |
| | | KDTLMISRTPEVTCVVVDVSHEDPEVKFNWYVD |
| | | GVEVHNAKTKPREEQYNSTYRVVSVLTVLHQDW |
| | | LNGKEYKCKVSNKALGAPIEKTISKAKGQPREPQ |
| | | VYTLPPCRDELTKNQVSLWCLVKGFYPSDIAVEW |
| | | ESNGQPENNYKTTPPVLDSDGSFFLYSKLTVDKS |
| | | RWQQGNVFSCSVMHEALHNHYTQKSLSLSPG |
| IL-7Rα_5 heavy chain | 214 | QVQLVESGGGLVQPGGSLRLSCAASGFTFSQRIM |
| (knob chain) | | SWYRQAPGKEHELVASISPFGDGTRYADSVKGRF |
| | | TISRDNARNQLYLQMNSLEFEDTAVYYCNKRNL |
| | | DVSDDWGQGTQVTVSSGGSGGGGSGGGGSGGG |
| | | GSGGGGSGGDKTHTCPPCPAPEAAGGPSVFLFPP |
| | | KPKDTLMISRTPEVTCVVVDVSHEDPEVKFNWY |
| | | VDGVEVHNAKTKPREEQYNSTYRVVSVLTVLHQ |
| | | DWLNGKEYKCKVSNKALGAPIEKTISKAKGQPRE |
| | | PQVYTLPPCRDELTKNQVSLWCLVKGFYPSDIAV |
| | | EWESNGQPENNYKTTPPVLDSDGSFFLYSKLTVD |
| | | KSRWQQGNVFSCSVMHEALHNHYTQKSLSLSPG |
| IL-7Rα_6 heavy chain | 215 | QVQLVESGGGSVQPGGSLRLSCATSGSIFSLATMT |
| (knob chain) | | WYRQGPGKTRELVAVITWRGTTTYADSVKGRFTI |
| | | SRDNAKNTMDLQMNGLKPEDTAVYYCNVKWG |
| | | GGLNRAPFDYWGQGTQVTVSSGGSGGGGSGGG |
| | | GSGGGGSGGGGSGGDKTHTCPPCPAPEAAGGPSV |
| | | FLFPPKPKDTLMISRTPEVTCVVVDVSHEDPEVKF |
| | | NWYVDGVEVHNAKTKPREEQYNSTYRVVSVLT |
| | | VLHQDWLNGKEYKCKVSNKALGAPIEKTISKAK |
| | | GQPREPQVYTLPPCRDELTKNQVSLWCLVKGFYP |
| | | SDIAVEWESNGQPENNYKTTPPVLDSDGSFFLYS |
| | | KLTVDKSRWQQGNVFSCSVMHEALHNHYTQKSL |
| | | SLSPG |
| IL-7Rα_7 heavy chain | 216 | QVQLVESGGGLVQPGGSLRLSCAASGFTFSNYAM |
| (knob chain) | | SWYRQAPGKERELVASISVFGGGTNYADSVKGRF |
| | | TISRDNAKNTVYLQMNSLKPEDSAVYYCNKRRY |
| | | DDYWGQGTQVTVSSGGSGGGGSGGGGSGGGGS |
| | | GGGGSGGDKTHTCPPCPAPEAAGGPSVFLFPPKP |
| | | KDTLMISRTPEVTCVVVDVSHEDPEVKFNWYVD |
| | | GVEVHNAKTKPREEQYNSTYRVVSVLTVLHQDW |
| | | LNGKEYKCKVSNKALGAPIEKTISKAKGQPREPQ |
| | | VYTLPPCRDELTKNQVSLWCLVKGFYPSDIAVEW |
| | | ESNGQPENNYKTTPPVLDSDGSFFLYSKLTVDKS |
| | | RWQQGNVFSCSVMHEALHNHYTQKSLSLSPG |
| IL-7Rα_8 heavy chain | 217 | QVQLVESGGGLVQPGGSLRLSCAASGFTISSWSM |
| (knob chain) | | KWVRQAPGKGPEWVSYISPGGGSLDYLDSVKGR |
| | | FTISRDNAKNTVYLQMSNLKSEDTALYWCATGT |
| | | GPTRGQGTQVTVSSGGSGGGGSGGGGSGGGGSG |
| | | GGGSGGDKTHTCPPCPAPEAAGGPSVFLFPPKPK |
| | | DTLMISRTPEVTCVVVDVSHEDPEVKFNWYVDG |
| | | VEVHNAKTKPREEQYNSTYRVVSVLTVLHQDWL |
| | | NGKEYKCKVSNKALGAPIEKTISKAKGQPREPQV |
| | | YTLPPCRDELTKNQVSLWCLVKGFYPSDIAVEWE |
| | | SNGQPENNYKTTPPVLDSDGSFFLYSKLTVDKSR |
| | | WQQGNVFSCSVMHEALHNHYTQKSLSLSPG |
| IL-7Rα_9 heavy chain | 218 | QVQLVESGGGLVQPGGSLRLSCAASGFTISTWSM |
| (knob chain) | | KWVRQAPGKGPEWVSYISPGGTSLDYQDSVKGR |
| | | FTISRDNAKNTVYLQMNNLKSEDTALYWCATGT |
| | | GETRGQGTQVTVSSGGSGGGGSGGGGSGGGGSG |
| | | GGGSGGDKTHTCPPCPAPEAAGGPSVFLFPPKPK |
| | | DTLMISRTPEVTCVVVDVSHEDPEVKFNWYVDG |
| | | VEVHNAKTKPREEQYNSTYRVVSVLTVLHQDWL |
| | | NGKEYKCKVSNKALGAPIEKTISKAKGQPREPQV |
| | | YTLPPCRDELTKNQVSLWCLVKGFYPSDIAVEWE |
| | | SNGQPENNYKTTPPVLDSDGSFFLYSKLTVDKSR |
| | | WQQGNVFSCSVMHEALHNHYTQKSLSLSPG |
| IL-7Rα_10 heavy chain | 219 | QVQLVESGGGLVQPGGSLRLSCATSGSIFSLATMT |
| (knob chain) | | WYRQAPGKTREFVAAITWRGAATYAESVKGRFT |
| | | ISRDNAKNTMDLQMDGLKPEDTAVYYCNVKWG |
| | | GGLNRAPFDYWGQGTQVTVSSGGSGGGGSGGG |
| | | GSGGGGSGGGGSGGDKTHTCPPCPAPEAAGGPSV |
| | | FLFPPKPKDTLMISRTPEVTCVVVDVSHEDPEVKF |
| | | NWYVDGVEVHNAKTKPREEQYNSTYRVVSVLT |
| | | VLHQDWLNGKEYKCKVSNKALGAPIEKTISKAK |
| | | GQPREPQVYTLPPCRDELTKNQVSLWCLVKGFYP |
| | | SDIAVEWESNGQPENNYKTTPPVLDSDGSFFLYS |
| | | KLTVDKSRWQQGNVFSCSVMHEALHNHYTQKSL |
| | | SLSPG |
| IL-7Rα_11 heavy chain | 220 | QVQLVESGGGLVQPGGSLRLSCEASGFTISSWSM |
| (knob chain) | | KWVRQAPGKGPEWVSYISPGGGSLDYHHSVKGR |
| | | FTISRDNARNTVYLQMSNLQSEDTALYWCATGT |
| | | GETRGQGTQVTVSSGGSGGGGSGGGGSGGGGSG |
| | | GGGSGGDKTHTCPPCPAPEAAGGPSVFLFPPKPK |
| | | DTLMISRTPEVTCVVVDVSHEDPEVKFNWYVDG |
| | | VEVHNAKTKPREEQYNSTYRVVSVLTVLHQDWL |
| | | NGKEYKCKVSNKALGAPIEKTISKAKGQPREPQV |
| | | YTLPPCRDELTKNQVSLWCLVKGFYPSDIAVEWE |
| | | SNGQPENNYKTTPPVLDSDGSFFLYSKLTVDKSR |
| | | WQQGNVFSCSVMHEALHNHYTQKSLSLSPG |
| IL-7Rα_12 heavy chain | 221 | QVQLVESGGGLVQPGGSLRLSCAASGFTISSRSM |
| (knob chain) | | KWVRQAPGKGPEWVSYISPGGGTVDYQDSVKGR |
| | | FTISRDNAKNTVYLQMSNLKSEDTALYWCATGT |
| | | GPTRGRGTQVTVSSGGSGGGGSGGGGSGGGGSG |
| | | GGGSGGDKTHTCPPCPAPEAAGGPSVFLFPPKPK |
| | | DTLMISRTPEVTCVVVDVSHEDPEVKFNWYVDG |
| | | VEVHNAKTKPREEQYNSTYRVVSVLTVLHQDWL |
| | | NGKEYKCKVSNKALGAPIEKTISKAKGQPREPQV |
| | | YTLPPCRDELTKNQVSLWCLVKGFYPSDIAVEWE |
| | | SNGQPENNYKTTPPVLDSDGSFFLYSKLTVDKSR |
| | | WQQGNVFSCSVMHEALHNHYTQKSLSLSPG |
| IL-7Rα_13 heavy chain | 222 | QVQLVESGGGLVRPGGSLRLACAASGIRFSLRNM |
| (knob chain) | | HWYRQASGRQREWVATITAGGVTGYRDSVKGR |
| | | FTISRDNAKNTVDLQMNGLQLEDTAVYYCNHGD |
| | | TAYWGQGTQVTVSSGGSGGGGSGGGGSGGGGS |
| | | GGGGSGGDKTHTCPPCPAPEAAGGPSVFLFPPKP |
| | | KDTLMISRTPEVTCVVVDVSHEDPEVKFNWYVD |
| | | GVEVHNAKTKPREEQYNSTYRVVSVLTVLHQDW |
| | | LNGKEYKCKVSNKALGAPIEKTISKAKGQPREPQ |
| | | VYTLPPCRDELTKNQVSLWCLVKGFYPSDIAVEW |
| | | ESNGQPENNYKTTPPVLDSDGSFFLYSKLTVDKS |
| | | RWQQGNVFSCSVMHEALHNHYTQKSLSLSPG |
| IL-7Rα_14 heavy chain | 223 | QVQLVESGGGLVQPGGSLRLSCAASGFTFSQRIM |
| (knob chain) | | SWYRQAPGKERELVASVSPFGDGTRYADSVKGR |
| | | FTISRDNAKSQLYLQMNNLKFEDTAVYYCNKRN |
| | | LEISDAWGQGTQVTVSSGGSGGGGSGGGGSGGG |
| | | GSGGGGSGGDKTHTCPPCPAPEAAGGPSVFLFPP |
| | | KPKDTLMISRTPEVTCVVVDVSHEDPEVKFNWY |
| | | VDGVEVHNAKTKPREEQYNSTYRVVSVLTVLHQ |
| | | DWLNGKEYKCKVSNKALGAPIEKTISKAKGQPRE |
| | | PQVYTLPPCRDELTKNQVSLWCLVKGFYPSDIAV |
| | | EWESNGQPENNYKTTPPVLDSDGSFFLYSKLTVD |
| | | KSRWQQGNVFSCSVMHEALHNHYTQKSLSLSPG |
| IL-7Rα_15 heavy chain | 224 | QVQLVESGGGLVQPGGSLRLSCAASGFTISSWSM |
| (knob chain) | | KWVRQAPGKGPEWVSYISPGGGSLDYHHSVKGR |
| | | FTISRDNARNTVYLQMSNLQSEDTALYWCATGT |
| | | GETRGQGTQVTVSSGGSGGGGSGGGGSGGGGSG |
| | | GGGSGGDKTHTCPPCPAPEAAGGPSVFLFPPKPK |
| | | DTLMISRTPEVTCVVVDVSHEDPEVKFNWYVDG |
| | | VEVHNAKTKPREEQYNSTYRVVSVLTVLHQDWL |
| | | NGKEYKCKVSNKALGAPIEKTISKAKGQPREPQV |
| | | YTLPPCRDELTKNQVSLWCLVKGFYPSDIAVEWE |
| | | SNGQPENNYKTTPPVLDSDGSFFLYSKLTVDKSR |
| | | WQQGNVFSCSVMHEALHNHYTQKSLSLSPG |
| IL-7Rα_16 heavy chain | 225 | QVQLVESGGGLVQPGGSLRLSCTASGSIFSLATMT |
| (knob chain) | | WYRQAPGKTREFVAAITWRGAATYAESVKGRFT |
| | | ISRDNAKNTMDLQMDGLKPEDTAVYYCNVKWG |
| | | GGLNRAPFDYWGQGTQVTVSSGGSGGGGSGGG |
| | | GSGGGGSGGGGSGGDKTHTCPPCPAPEAAGGPSV |
| | | FLFPPKPKDTLMISRTPEVTCVVVDVSHEDPEVKF |
| | | NWYVDGVEVHNAKTKPREEQYNSTYRVVSVLT |
| | | VLHQDWLNGKEYKCKVSNKALGAPIEKTISKAK |
| | | GQPREPQVYTLPPCRDELTKNQVSLWCLVKGFYP |
| | | SDIAVEWESNGQPENNYKTTPPVLDSDGSFFLYS |
| | | KLTVDKSRWQQGNVFSCSVMHEALHNHYTQKSL |
| | | SLSPG |
| IL-2Rγ_2 heavy chain | 226 | QVQLQESGGGLVQAGGSLRLSCAASISISKIDLMG |
| (hole chain) | | WYRQAPGRQRELVARVTNGGDSYYSTSVKGRFT |
| | | ISRDNAKNTLYLQMNSLKPEDTAVYYCYGVPESL |
| | | AAFHWGQGTLVTVSSGGSGGGGSGGGGSGGGGS |
| | | GGGGSGGDKTHTCPPCPAPEAAGGPSVFLFPPKP |
| | | KDTLMISRTPEVTCVVVDVSHEDPEVKFNWYVD |
| | | GVEVHNAKTKPREEQYNSTYRVVSVLTVLHQDW |
| | | LNGKEYKCKVSNKALGAPIEKTISKAKGQPREPQ |
| | | VCTLPPSRDELTKNQVSLSCAVKGFYPSDIAVEW |
| | | ESNGQPENNYKTTPPVLDSDGSFFLVSKLTVDKS |
| | | RWQQGNVFSCSVMHEALHNHYTQKSLSLSPG |
| IL-2Rγ_3 heavy chain | 227 | QVQLQESGGGLVQPGGSLRLSCAASGSIFSGNAM |
| (hole chain) | | GWYRQAPGKQRELVASITSGGDTHYVDSVKGRF |
| | | TISRDNAKNMVYLQMSSLKPEDTAVYYCNAQER |
| | | VYSDYAFASWGPGTQVTVSSGGSGGGGSGGGGS |
| | | GGGGSGGGGSGGDKTHTCPPCPAPEAAGGPSVFL |
| | | FPPKPKDTLMISRTPEVTCVVVDVSHEDPEVKFN |
| | | WYVDGVEVHNAKTKPREEQYNSTYRVVSVLTVL |
| | | HQDWLNGKEYKCKVSNKALGAPIEKTISKAKGQ |
| | | PREPQVCTLPPSRDELTKNQVSLSCAVKGFYPSDI |
| | | AVEWESNGQPENNYKTTPPVLDSDGSFFLVSKLT |
| | | VDKSRWQQGNVFSCSVMHEALHNHYTQKSLSLS |
| | | PG |
| IL-2Rγ_4 heavy chain | 228 | QVQLQESGGGLVQPGGSLRLSCAASGFTFSTYVM |
| (hole chain) | | NWVRQAPGKGLEWVSGIDSDGDSTTYTDSVKGR |
| | | FTISRDNAKNTLYLQMNSLKPEDTAVYYCSMFR |
| | | MATTGSQGTQVTVSSGGSGGGGSGGGGSGGGGS |
| | | GGGGSGGDKTHTCPPCPAPEAAGGPSVFLFPPKP |
| | | KDTLMISRTPEVTCVVVDVSHEDPEVKFNWYVD |
| | | GVEVHNAKTKPREEQYNSTYRVVSVLTVLHQDW |
| | | LNGKEYKCKVSNKALGAPIEKTISKAKGQPREPQ |
| | | VCTLPPSRDELTKNQVSLSCAVKGFYPSDIAVEW |
| | | ESNGQPENNYKTTPPVLDSDGSFFLVSKLTVDKS |
| | | RWQQGNVFSCSVMHEALHNHYTQKSLSLSPG |
| IL-2Rγ_5 heavy chain | 229 | QVQLQESGGGLVQAGGSLRLSCAASGITSSIYAM |
| (hole chain) | | GWYRQAPGNEREPVALITSGDNTNYPDSVKGRFT |
| | | ISRDTAKNTVYLQMSSLKPEDTAVYYCYADVVIG |
| | | TTYYTSWGQGTLVTVSSGGSGGGGSGGGGSGGG |
| | | GSGGGGSGGDKTHTCPPCPAPEAAGGPSVFLFPP |
| | | KPKDTLMISRTPEVTCVVVDVSHEDPEVKFNWY |
| | | VDGVEVHNAKTKPREEQYNSTYRVVSVLTVLHQ |
| | | DWLNGKEYKCKVSNKALGAPIEKTISKAKGQPRE |
| | | PQVCTLPPSRDELTKNQVSLSCAVKGFYPSDIAVE |
| | | WESNGQPENNYKTTPPVLDSDGSFFLVSKLTVDK |
| | | SRWQQGNVFSCSVMHEALHNHYTQKSLSLSPG |
| non-binding DP47 | 230 | EIVLTQSPGTLSLSPGERATLSCRASQSVSSSYLA |
| antibody LC | | WYQQKPGQAPRLLIYGASSRATGIPDRFSGSGSGT |
| (P1AD3966) | | DFTLTISRLEPEDFAVYYCQQYGSSPLTFGQGTKV |
| | | EIKRTVAAPSVFIFPPSDEQLKSGTASVVCLLNNF |
| | | YPREAKVQWKVDNALQSGNSQESVTEQDSKDST |
| | | YSLSSTLTLSKADYEKHKVYACEVTHQGLSSPVT |
| | | KSFNRGEC |
| non-binding DP47 | 231 | EVQLLESGGGLVQPGGSLRLSCAASGFTFSSYAM |
| antibody HC | | SWVRQAPGKGLEWVSAISGSGGSTYYADSVKGR |
| (P1AD3966) | | FTISRDNSKNTLYLQMNSLRAEDTAVYYCAKGSG |
| | | FDYWGQGTLVTVSSASTKGPSVFPLAPSSKSTSG |
| | | GTAALGCLVKDYFPEPVTVSWNSGALTSGVHTFP |
| | | AVLQSSGLYSLSSVVTVPSSSLGTQTYICNVNHKP |
| | | SNTKVDKKVEPKSCDKTHTCPPCPAPELLGGPSV |
| | | FLFPPKPKDTLMISRTPEVTCVVVDVSHEDPEVKF |
| | | NWYVDGVEVHNAKTKPREEQYNSTYRVVSVLT |
| | | VLHQDWLNGKEYKCKVSNKALPAPIEKTISKAKG |
| | | QPREPQVYTLPPSRDELTKNQVSLTCLVKGFYPSD |
| | | IAVEWESNGQPENNYKTTPPVLDSDGSFFLYSKLT |
| | | VDKSRWQQGNVFSCSVMHEALHNHYTQKSLSLS |
| | | PGGGSGGLNDIFEAQKIEWHE |
| FolR1-TCB knob chain | 232 | EVQLLESGGGLVQPGGSLRLSCAASGFTFSSYAM |
| (P1AK1120) | | NWVRQAPGKGLEWVSRIRSKYNNYATYYADSV |
| | | KGRFTISRDDSKNTLYLQMNSLRAEDTAVYYCVR |
| | | ASNFPASYVSYFAYWGQGTLVTVSSASTKGPSVF |
| | | PLAPSSKSTSGGTAALGCLVKDYFPEPVTVSWNS |
| | | GALTSGVHTFPAVLQSSGLYSLSSVVTVPSSSLGT |
| | | QTYICNVNHKPSNTKVDKKVEPKSCDGGGGSGG |
| | | GGSEVQLVESGGGLVKPGGSLRLSCAASGFTFSN |
| | | AWMSWVRQAPGKGLEWVGRIKSKTDGGTTDYA |
| | | APVKGRFTISRDDSKNTLYLQMNSLKTEDTAVYY |
| | | CTTPWEWSWYDYWGQGTLVTVSSASTKGPSVFP |
| | | LAPSSKSTSGGTAALGCLVKDYFPEPVTVSWNSG |
| | | ALTSGVHTFPAVLQSSGLYSLSSVVTVPSSSLGTQ |
| | | TYICNVNHKPSNTKVDKKVEPKSCDKTHTCPPCP |
| | | APEAAGGPSVFLFPPKPKDTLMISRTPEVTCVVVD |
| | | VSHEDPEVKFNWYVDGVEVHNAKTKPREEQYNS |
| | | TYRVVSVLTVLHQDWLNGKEYKCKVSNKALGA |
| | | PIEKTISKAKGQPREPQVYTLPPCRDELTKNQVSL |
| | | WCLVKGFYPSDIAVEWESNGQPENNYKTTPPVLD |
| | | SDGSFFLYSKLTVDKSRWQQGNVFSCSVMHEAL |
| | | HNHYTQKSLSLSPGK |
| FolR1-TCB hole chain | 233 | EVQLVESGGGLVKPGGSLRLSCAASGFTFSNAW |
| (P1AK1120) | | MSWVRQAPGKGLEWVGRIKSKTDGGTTDYAAP |
| | | VKGRFTISRDDSKNTLYLQMNSLKTEDTAVYYCT |
| | | TPWEWSWYDYWGQGTLVTVSSASTKGPSVFPLA |
| | | PSSKSTSGGTAALGCLVKDYFPEPVTVSWNSGAL |
| | | TSGVHTFPAVLQSSGLYSLSSVVTVPSSSLGTQTYI |
| | | CNVNHKPSNTKVDKKVEPKSCDKTHTCPPCPAPE |
| | | AAGGPSVFLFPPKPKDTLMISRTPEVTCVVVDVSH |
| | | EDPEVKFNWYVDGVEVHNAKTKPREEQYNSTYR |
| | | VVSVLTVLHQDWLNGKEYKCKVSNKALGAPIEK |
| | | TISKAKGQPREPQVCTLPPSRDELTKNQVSLSCAV |
| | | KGFYPSDIAVEWESNGQPENNYKTTPPVLDSDGS |
| | | FFLVSKLTVDKSRWQQGNVFSCSVMHEALHNHY |
| | | TQKSLSLSPGK |
| FolR1-TCB LC | 234 | QAVVTQEPSLTVSPGGTVTLTCGSSTGAVTTSNY |
| (P1AK1120) | | ANWVQEKPGQAFRGLIGGTNKRAPGTPARFSGSL |
| | | LGGKAALTLSGAQPEDEAEYYCALWYSNLWVFG |
| | | GGTKLTVLGQPKAAPSVTLFPPSSEELQANKATL |
| | | VCLISDFYPGAVTVAWKADSSPVKAGVETTTPSK |
| | | QSNNKYAASSYLSLTPEQWKSHRSYSCQVTHEGS |
| | | TVEKTVAPTECS |
| hole chain (P1AJ8560, | 235 | QVQLQQSGGGLVQAGGSLRLSCTASGSTFSFSNY |
| P1AJ8561) | | HMGWYRQAPGKQRERVASISSRDSTYYSDSVKG |
| | | RFTISRDTARNTVYLQMNSLEPEETAVYYCNARG |
| | | RATGRDYWGQGTQVTVSSGGSGGGGSGGGGSG |
| | | GDKTHTCPPCPAPEAAGGPSVFLFPPKPKDTLMIS |
| | | RTPEVTCVVVDVSHEDPEVKFNWYVDGVEVHNA |
| | | KTKPREEQYNSTYRVVSVLTVLHQDWLNGKEYK |
| | | CKVSNKALGAPIEKTISKAKGQPREPQVCTLPPSR |
| | | DELTKNQVSLSCAVKGFYPSDIAVEWESNGQPEN |
| | | NYKTTPPVLDSDGSFFLVSKLTVDKSRWQQGNVF |
| | | SCSVMHEALHNRFTQKSLSLSPG |
| hole chain (P1AJ8562, | 236 | QVQLQEFGGGLVQAGEALRLSCVASKTIFNTMP |
| P1AJ8563) | | MGWYRQAPGKERELVATITSSGVVNSADSVKGR |
| | | FTISRDSAKRTAYLQMNNLKPEDTAVYYCAAMF |
| | | KPGIPEYWGRGTQVTVSSGGSGGGGSGGGGSGG |
| | | DKTHTCPPCPAPEAAGGPSVFLFPPKPKDTLMISR |
| | | TPEVTCVVVDVSHEDPEVKFNWYVDGVEVHNA |
| | | KTKPREEQYNSTYRVVSVLTVLHQDWLNGKEYK |
| | | CKVSNKALGAPIEKTISKAKGQPREPQVCTLPPSR |
| | | DELTKNQVSLSCAVKGFYPSDIAVEWESNGQPEN |
| | | NYKTTPPVLDSDGSFFLVSKLTVDKSRWQQGNVF |
| | | SCSVMHEALHNRFTQKSLSLSPG |
| hole chain (P1AJ8564, | 237 | QVQLQQSGGGLVQAGGSLRLSCTASGSTFSFSNY |
| P1AJ8565) | | HMGWYRQAPGKQRERVASISSRDSTYYSDSVKG |
| | | RFTISRDTARNTVYLQMNSLEPEETAVYYCNARG |
| | | RATGRDYWGQGTQVTVSSGGSGGDKTHTCPPCP |
| | | APEAAGGPSVFLFPPKPKDTLMISRTPEVTCVVVD |
| | | VSHEDPEVKFNWYVDGVEVHNAKTKPREEQYNS |
| | | TYRVVSVLTVLHQDWLNGKEYKCKVSNKALGA |
| | | PIEKTISKAKGQPREPQVCTLPPSRDELTKNQVSLS |
| | | CAVKGFYPSDIAVEWESNGQPENNYKTTPPVLDS |
| | | DGSFFLVSKLTVDKSRWQQGNVFSCSVMHEALH |
| | | NRFTQKSLSLSPG |
| hole chain (P1AJ8566, | 238 | QVQLQEFGGGLVQAGEALRLSCVASKTIFNTMP |
| P1AJ8567) | | MGWYRQAPGKERELVATITSSGVVNSADSVKGR |
| | | FTISRDSAKRTAYLQMNNLKPEDTAVYYCAAMF |
| | | KPGIPEYWGRGTQVTVSSGGSGGDKTHTCPPCPA |
| | | PEAAGGPSVFLFPPKPKDTLMISRTPEVTCVVVDV |
| | | SHEDPEVKFNWYVDGVEVHNAKTKPREEQYNST |
| | | YRVVSVLTVLHQDWLNGKEYKCKVSNKALGAPI |
| | | EKTISKAKGQPREPQVCTLPPSRDELTKNQVSLSC |
| | | AVKGFYPSDIAVEWESNGQPENNYKTTPPVLDSD |
| | | GSFFLVSKLTVDKSRWQQGNVFSCSVMHEALHN |
| | | RFTQKSLSLSPG |
| hole chain (P1AJ8568, | 239 | QVQLQQSGGGLVQAGGSLRLSCTASGSTFSFSNY |
| P1AJ8569) | | HMGWYRQAPGKQRERVASISSRDSTYYSDSVKG |
| | | RFTISRDTARNTVYLQMNSLEPEETAVYYCNARG |
| | | RATGRDYWGQGTQVTVSSGGSGGGGSGGGGSG |
| | | GGGSGGGGSGGDKTHTCPPCPAPEAAGGPSVFLF |
| | | PPKPKDTLMISRTPEVTCVVVDVSHEDPEVKFNW |
| | | YVDGVEVHNAKTKPREEQYNSTYRVVSVLTVLH |
| | | QDWLNGKEYKCKVSNKALGAPIEKTISKAKGQP |
| | | REPQVCTLPPSRDELTKNQVSLSCAVKGFYPSDIA |
| | | VEWESNGQPENNYKTTPPVLDSDGSFFLVSKLTV |
| | | DKSRWQQGNVFSCSVMHEALHNRFTQKSLSLSP |
| | | G |
| hole chain (P1AJ8570, | 240 | QVQLQEFGGGLVQAGEALRLSCVASKTIFNTMP |
| P1AJ8571) | | MGWYRQAPGKERELVATITSSGVVNSADSVKGR |
| | | FTISRDSAKRTAYLQMNNLKPEDTAVYYCAAMF |
| | | KPGIPEYWGRGTQVTVSSGGSGGGGSGGGGSGG |
| | | GGSGGGGSGGDKTHTCPPCPAPEAAGGPSVFLFP |
| | | PKPKDTLMISRTPEVTCVVVDVSHEDPEVKFNWY |
| | | VDGVEVHNAKTKPREEQYNSTYRVVSVLTVLHQ |
| | | DWLNGKEYKCKVSNKALGAPIEKTISKAKGQPRE |
| | | PQVCTLPPSRDELTKNQVSLSCAVKGFYPSDIAVE |
| | | WESNGQPENNYKTTPPVLDSDGSFFLVSKLTVDK |
| | | SRWQQGNVFSCSVMHEALHNRFTQKSLSLSPG |
| hole chain (P1AJ8572 - | 241 | DKTHTCPPCPAPEAAGGPSVFLFPPKPKDTLMISR |
| P1AJ8583) | | TPEVTCVVVDVSHEDPEVKFNWYVDGVEVHNA |
| | | KTKPREEQYNSTYRVVSVLTVLHQDWLNGKEYK |
| | | CKVSNKALGAPIEKTISKAKGQPREPQVCTLPPSR |
| | | DELTKNQVSLSCAVKGFYPSDIAVEWESNGQPEN |
| | | NYKTTPPVLDSDGSFFLVSKLTVDKSRWQQGNVF |
| | | SCSVMHEALHNRFTQKSLSLSPG |
| knob chain (P1AJ8572) | 242 | EVQLLESGGGLVQPGGSLRLSCAASGFTFSSYAM |
| | | SWVRQAPGKGLEWVSAIIGSGASTYYADSVKGRF |
| | | TISRDNSKNTLYLQMNSLRAEDTAVYYCAKGWF |
| | | GGFNYWGQGTLVTVSSASTKGPSVFPLAPSSKST |
| | | SGGTAALGCLVKDYFPEPVTVSWNSGALTSGVH |
| | | TFPAVLQSSGLYSLSSVVTVPSSSLGTQTYICNVN |
| | | HKPSNTKVDKKVEPKSCDKTHTCPPCPAPEAAGG |
| | | PSVFLFPPKPKDTLMISRTPEVTCVVVDVSHEDPE |
| | | VKFNWYVDGVEVHNAKTKPREEQYNSTYRVVS |
| | | VLTVLHQDWLNGKEYKCKVSNKALGAPIEKTISK |
| | | AKGQPREPQVYTLPPCRDELTKNQVSLWCLVKG |
| | | FYPSDIAVEWESNGQPENNYKTTPPVLDSDGSFFL |
| | | YSKLTVDKSRWQQGNVFSCSVMHEALHNHYTQ |
| | | KSLSLSPGGGSGGGGSGGGGSGGQVQLQQSGGG |
| | | LVQAGGSLRLSCTASGSTFSFSNYHMGWYRQAP |
| | | GKQRERVASISSRDSTYYSDSVKGRFTISRDTARN |
| | | TVYLQMNSLEPEETAVYYCNARGRATGRDYWG |
| | | QGTQVTVSS |
| knob chain (P1AJ8573) | 243 | QVQLVQSGAEVKKPGASVKVSCKASGYTLTDYN |
| | | MDWVRQAPGQGLEWIGDIYPNTGGTIYNQKFKG |
| | | RVTMTIDTSTSTVYMELSSLRSEDTAVYYCTRFR |
| | | GIHYAMDYWGQGTTVTVSSASTKGPSVFPLAPSS |
| | | KSTSGGTAALGCLVKDYFPEPVTVSWNSGALTSG |
| | | VHTFPAVLQSSGLYSLSSVVTVPSSSLGTQTYICN |
| | | VNHKPSNTKVDKKVEPKSCDKTHTCPPCPAPEAA |
| | | GGPSVFLFPPKPKDTLMISRTPEVTCVVVDVSHED |
| | | PEVKFNWYVDGVEVHNAKTKPREEQYNSTYRVV |
| | | SVLTVLHQDWLNGKEYKCKVSNKALGAPIEKTIS |
| | | KAKGQPREPQVYTLPPCRDELTKNQVSLWCLVK |
| | | GFYPSDIAVEWESNGQPENNYKTTPPVLDSDGSFF |
| | | LYSKLTVDKSRWQQGNVFSCSVMHEALHNHYTQ |
| | | KSLSLSPGGGSGGGGSGGGGSGGQVQLQQSGGG |
| | | LVQAGGSLRLSCTASGSTFSFSNYHMGWYRQAP |
| | | GKQRERVASISSRDSTYYSDSVKGRFTISRDTARN |
| | | TVYLQMNSLEPEETAVYYCNARGRATGRDYWG |
| | | QGTQVTVSS |
| knob chain (P1AJ8574) | 244 | EVQLLESGGGLVQPGGSLRLSCAASGFTFSSYAM |
| | | SWVRQAPGKGLEWVSAIIGSGASTYYADSVKGRF |
| | | TISRDNSKNTLYLQMNSLRAEDTAVYYCAKGWF |
| | | GGFNYWGQGTLVTVSSASTKGPSVFPLAPSSKST |
| | | SGGTAALGCLVKDYFPEPVTVSWNSGALTSGVH |
| | | TFPAVLQSSGLYSLSSVVTVPSSSLGTQTYICNVN |
| | | HKPSNTKVDKKVEPKSCDKTHTCPPCPAPEAAGG |
| | | PSVFLFPPKPKDTLMISRTPEVTCVVVDVSHEDPE |
| | | VKFNWYVDGVEVHNAKTKPREEQYNSTYRVVS |
| | | VLTVLHQDWLNGKEYKCKVSNKALGAPIEKTISK |
| | | AKGQPREPQVYTLPPCRDELTKNQVSLWCLVKG |
| | | FYPSDIAVEWESNGQPENNYKTTPPVLDSDGSFFL |
| | | YSKLTVDKSRWQQGNVFSCSVMHEALHNHYTQ |
| | | KSLSLSPGGGSGGGGSGGGGSGGQVQLQEFGGG |
| | | LVQAGEALRLSCVASKTIFNTMPMGWYRQAPGK |
| | | ERELVATITSSGVVNSADSVKGRFTISRDSAKRTA |
| | | YLQMNNLKPEDTAVYYCAAMFKPGIPEYWGRGT |
| | | QVTVSS |
| knob chain (P1AJ8575) | 245 | QVQLVQSGAEVKKPGASVKVSCKASGYTLTDYN |
| | | MDWVRQAPGQGLEWIGDIYPNTGGTIYNQKFKG |
| | | RVTMTIDTSTSTVYMELSSLRSEDTAVYYCTRFR |
| | | GIHYAMDYWGQGTTVTVSSASTKGPSVFPLAPSS |
| | | KSTSGGTAALGCLVKDYFPEPVTVSWNSGALTSG |
| | | VHTFPAVLQSSGLYSLSSVVTVPSSSLGTQTYICN |
| | | VNHKPSNTKVDKKVEPKSCDKTHTCPPCPAPEAA |
| | | GGPSVFLFPPKPKDTLMISRTPEVTCVVVDVSHED |
| | | PEVKFNWYVDGVEVHNAKTKPREEQYNSTYRVV |
| | | SVLTVLHQDWLNGKEYKCKVSNKALGAPIEKTIS |
| | | KAKGQPREPQVYTLPPCRDELTKNQVSLWCLVK |
| | | GFYPSDIAVEWESNGQPENNYKTTPPVLDSDGSFF |
| | | LYSKLTVDKSRWQQGNVFSCSVMHEALHNHYTQ |
| | | KSLSLSPGGGSGGGGSGGGGSGGQVQLQEFGGG |
| | | LVQAGEALRLSCVASKTIFNTMPMGWYRQAPGK |
| | | ERELVATITSSGVVNSADSVKGRFTISRDSAKRTA |
| | | YLQMNNLKPEDTAVYYCAAMFKPGIPEYWGRGT |
| | | QVTVSS |
| knob chain (P1AJ8576) | 246 | EVQLLESGGGLVQPGGSLRLSCAASGFTFSSYAM |
| | | SWVRQAPGKGLEWVSAIIGSGASTYYADSVKGRF |
| | | TISRDNSKNTLYLQMNSLRAEDTAVYYCAKGWF |
| | | GGFNYWGQGTLVTVSSASTKGPSVFPLAPSSKST |
| | | SGGTAALGCLVKDYFPEPVTVSWNSGALTSGVH |
| | | TFPAVLQSSGLYSLSSVVTVPSSSLGTQTYICNVN |
| | | HKPSNTKVDKKVEPKSCDKTHTCPPCPAPEAAGG |
| | | PSVFLFPPKPKDTLMISRTPEVTCVVVDVSHEDPE |
| | | VKFNWYVDGVEVHNAKTKPREEQYNSTYRVVS |
| | | VLTVLHQDWLNGKEYKCKVSNKALGAPIEKTISK |
| | | AKGQPREPQVYTLPPCRDELTKNQVSLWCLVKG |
| | | FYPSDIAVEWESNGQPENNYKTTPPVLDSDGSFFL |
| | | YSKLTVDKSRWQQGNVFSCSVMHEALHNHYTQ |
| | | KSLSLSPGGGSGGQVQLQQSGGGLVQAGGSLRLS |
| | | CTASGSTFSFSNYHMGWYRQAPGKQRERVASISS |
| | | RDSTYYSDSVKGRFTISRDTARNTVYLQMNSLEP |
| | | EETAVYYCNARGRATGRDYWGQGTQVTVSS |
| knob chain (P1AJ8577) | 247 | QVQLVQSGAEVKKPGASVKVSCKASGYTLTDYN |
| | | MDWVRQAPGQGLEWIGDIYPNTGGTIYNQKFKG |
| | | RVTMTIDTSTSTVYMELSSLRSEDTAVYYCTRFR |
| | | GIHYAMDYWGQGTTVTVSSASTKGPSVFPLAPSS |
| | | KSTSGGTAALGCLVKDYFPEPVTVSWNSGALTSG |
| | | VHTFPAVLQSSGLYSLSSVVTVPSSSLGTQTYICN |
| | | VNHKPSNTKVDKKVEPKSCDKTHTCPPCPAPEAA |
| | | GGPSVFLFPPKPKDTLMISRTPEVTCVVVDVSHED |
| | | PEVKFNWYVDGVEVHNAKTKPREEQYNSTYRVV |
| | | SVLTVLHQDWLNGKEYKCKVSNKALGAPIEKTIS |
| | | KAKGQPREPQVYTLPPCRDELTKNQVSLWCLVK |
| | | GFYPSDIAVEWESNGQPENNYKTTPPVLDSDGSFF |
| | | LYSKLTVDKSRWQQGNVFSCSVMHEALHNHYTQ |
| | | KSLSLSPGGGSGGQVQLQQSGGGLVQAGGSLRLS |
| | | CTASGSTFSFSNYHMGWYRQAPGKQRERVASISS |
| | | RDSTYYSDSVKGRFTISRDTARNTVYLQMNSLEP |
| | | EETAVYYCNARGRATGRDYWGQGTQVTVSS |
| knob chain (P1AJ8578) | 248 | EVQLLESGGGLVQPGGSLRLSCAASGFTFSSYAM |
| | | SWVRQAPGKGLEWVSAIIGSGASTYYADSVKGRF |
| | | TISRDNSKNTLYLQMNSLRAEDTAVYYCAKGWF |
| | | GGFNYWGQGTLVTVSSASTKGPSVFPLAPSSKST |
| | | SGGTAALGCLVKDYFPEPVTVSWNSGALTSGVH |
| | | TFPAVLQSSGLYSLSSVVTVPSSSLGTQTYICNVN |
| | | HKPSNTKVDKKVEPKSCDKTHTCPPCPAPEAAGG |
| | | PSVFLFPPKPKDTLMISRTPEVTCVVVDVSHEDPE |
| | | VKFNWYVDGVEVHNAKTKPREEQYNSTYRVVS |
| | | VLTVLHQDWLNGKEYKCKVSNKALGAPIEKTISK |
| | | AKGQPREPQVYTLPPCRDELTKNQVSLWCLVKG |
| | | FYPSDIAVEWESNGQPENNYKTTPPVLDSDGSFFL |
| | | YSKLTVDKSRWQQGNVFSCSVMHEALHNHYTQ |
| | | KSLSLSPGGGSGGQVQLQEFGGGLVQAGEALRLS |
| | | CVASKTIFNTMPMGWYRQAPGKERELVATITSSG |
| | | VVNSADSVKGRFTISRDSAKRTAYLQMNNLKPED |
| | | TAVYYCAAMFKPGIPEYWGRGTQVTVSS |
| knob chain (P1AJ8579) | 249 | QVQLVQSGAEVKKPGASVKVSCKASGYTLTDYN |
| | | MDWVRQAPGQGLEWIGDIYPNTGGTIYNQKFKG |
| | | RVTMTIDTSTSTVYMELSSLRSEDTAVYYCTRFR |
| | | GIHYAMDYWGQGTTVTVSSASTKGPSVFPLAPSS |
| | | KSTSGGTAALGCLVKDYFPEPVTVSWNSGALTSG |
| | | VHTFPAVLQSSGLYSLSSVVTVPSSSLGTQTYICN |
| | | VNHKPSNTKVDKKVEPKSCDKTHTCPPCPAPEAA |
| | | GGPSVFLFPPKPKDTLMISRTPEVTCVVVDVSHED |
| | | PEVKFNWYVDGVEVHNAKTKPREEQYNSTYRVV |
| | | SVLTVLHQDWLNGKEYKCKVSNKALGAPIEKTIS |
| | | KAKGQPREPQVYTLPPCRDELTKNQVSLWCLVK |
| | | GFYPSDIAVEWESNGQPENNYKTTPPVLDSDGSFF |
| | | LYSKLTVDKSRWQQGNVFSCSVMHEALHNHYTQ |
| | | KSLSLSPGGGSGGQVQLQEFGGGLVQAGEALRLS |
| | | CVASKTIFNTMPMGWYRQAPGKERELVATITSSG |
| | | VVNSADSVKGRFTISRDSAKRTAYLQMNNLKPED |
| | | TAVYYCAAMFKPGIPEYWGRGTQVTVSS |
| knob chain (P1AJ8580) | 250 | EVQLLESGGGLVQPGGSLRLSCAASGFTFSSYAM |
| | | SWVRQAPGKGLEWVSAIIGSGASTYYADSVKGRF |
| | | TISRDNSKNTLYLQMNSLRAEDTAVYYCAKGWF |
| | | GGFNYWGQGTLVTVSSASTKGPSVFPLAPSSKST |
| | | SGGTAALGCLVKDYFPEPVTVSWNSGALTSGVH |
| | | TFPAVLQSSGLYSLSSVVTVPSSSLGTQTYICNVN |
| | | HKPSNTKVDKKVEPKSCDKTHTCPPCPAPEAAGG |
| | | PSVFLFPPKPKDTLMISRTPEVTCVVVDVSHEDPE |
| | | VKFNWYVDGVEVHNAKTKPREEQYNSTYRVVS |
| | | VLTVLHQDWLNGKEYKCKVSNKALGAPIEKTISK |
| | | AKGQPREPQVYTLPPCRDELTKNQVSLWCLVKG |
| | | FYPSDIAVEWESNGQPENNYKTTPPVLDSDGSFFL |
| | | YSKLTVDKSRWQQGNVFSCSVMHEALHNHYTQ |
| | | KSLSLSPGGGSGGGGSGGGGSGGGGSGGGGSGG |
| | | QVQLQQSGGGLVQAGGSLRLSCTASGSTFSFSNY |
| | | HMGWYRQAPGKQRERVASISSRDSTYYSDSVKG |
| | | RFTISRDTARNTVYLQMNSLEPEETAVYYCNARG |
| | | RATGRDYWGQGTQVTVSS |
| knob chain (P1AJ8581) | 251 | QVQLVQSGAEVKKPGASVKVSCKASGYTLTDYN |
| | | MDWVRQAPGQGLEWIGDIYPNTGGTIYNQKFKG |
| | | RVTMTIDTSTSTVYMELSSLRSEDTAVYYCTRFR |
| | | GIHYAMDYWGQGTTVTVSSASTKGPSVFPLAPSS |
| | | KSTSGGTAALGCLVKDYFPEPVTVSWNSGALTSG |
| | | VHTFPAVLQSSGLYSLSSVVTVPSSSLGTQTYICN |
| | | VNHKPSNTKVDKKVEPKSCDKTHTCPPCPAPEAA |
| | | GGPSVFLFPPKPKDTLMISRTPEVTCVVVDVSHED |
| | | PEVKFNWYVDGVEVHNAKTKPREEQYNSTYRVV |
| | | SVLTVLHQDWLNGKEYKCKVSNKALGAPIEKTIS |
| | | KAKGQPREPQVYTLPPCRDELTKNQVSLWCLVK |
| | | GFYPSDIAVEWESNGQPENNYKTTPPVLDSDGSFF |
| | | LYSKLTVDKSRWQQGNVFSCSVMHEALHNHYTQ |
| | | KSLSLSPGGGSGGGGSGGGGSGGGGSGGGGSGG |
| | | QVQLQQSGGGLVQAGGSLRLSCTASGSTFSFSNY |
| | | HMGWYRQAPGKQRERVASISSRDSTYYSDSVKG |
| | | RFTISRDTARNTVYLQMNSLEPEETAVYYCNARG |
| | | RATGRDYWGQGTQVTVSS |
| knob chain | 252 | EVQLLESGGGLVQPGGSLRLSCAASGFTFSSYAM |
| (P1AJ8582) | | SWVRQAPGKGLEWVSAIIGSGASTYYADSVKGRF |
| | | TISRDNSKNTLYLQMNSLRAEDTAVYYCAKGWF |
| | | GGFNYWGQGTLVTVSSASTKGPSVFPLAPSSKST |
| | | SGGTAALGCLVKDYFPEPVTVSWNSGALTSGVH |
| | | TFPAVLQSSGLYSLSSVVTVPSSSLGTQTYICNVN |
| | | HKPSNTKVDKKVEPKSCDKTHTCPPCPAPEAAGG |
| | | PSVFLFPPKPKDTLMISRTPEVTCVVVDVSHEDPE |
| | | VKFNWYVDGVEVHNAKTKPREEQYNSTYRVVS |
| | | VLTVLHQDWLNGKEYKCKVSNKALGAPIEKTISK |
| | | AKGQPREPQVYTLPPCRDELTKNQVSLWCLVKG |
| | | FYPSDIAVEWESNGQPENNYKTTPPVLDSDGSFFL |
| | | YSKLTVDKSRWQQGNVFSCSVMHEALHNHYTQ |
| | | KSLSLSPGGGSGGGGSGGGGSGGGGSGGGGSGG |
| | | QVQLQEFGGGLVQAGEALRLSCVASKTIFNTMP |
| | | MGWYRQAPGKERELVATITSSGVVNSADSVKGR |
| | | FTISRDSAKRTAYLQMNNLKPEDTAVYYCAAMF |
| | | KPGIPEYWGRGTQVTVSS |
| knob chain (P1AJ8583) | 253 | QVQLVQSGAEVKKPGASVKVSCKASGYTLTDYN |
| | | MDWVRQAPGQGLEWIGDIYPNTGGTIYNQKFKG |
| | | RVTMTIDTSTSTVYMELSSLRSEDTAVYYCTRFR |
| | | GIHYAMDYWGQGTTVTVSSASTKGPSVFPLAPSS |
| | | KSTSGGTAALGCLVKDYFPEPVTVSWNSGALTSG |
| | | VHTFPAVLQSSGLYSLSSVVTVPSSSLGTQTYICN |
| | | VNHKPSNTKVDKKVEPKSCDKTHTCPPCPAPEAA |
| | | GGPSVFLFPPKPKDTLMISRTPEVTCVVVDVSHED |
| | | PEVKFNWYVDGVEVHNAKTKPREEQYNSTYRVV |
| | | SVLTVLHQDWLNGKEYKCKVSNKALGAPIEKTIS |
| | | KAKGQPREPQVYTLPPCRDELTKNQVSLWCLVK |
| | | GFYPSDIAVEWESNGQPENNYKTTPPVLDSDGSFF |
| | | LYSKLTVDKSRWQQGNVFSCSVMHEALHNHYTQ |
| | | KSLSLSPGGGSGGGGSGGGGSGGGGSGGGGSGG |
| | | QVQLQEFGGGLVQAGEALRLSCVASKTIFNTMP |
| | | MGWYRQAPGKERELVATITSSGVVNSADSVKGR |
| | | FTISRDSAKRTAYLQMNNLKPEDTAVYYCAAMF |
| | | KPGIPEYWGRGTQVTVSS |
| LC (P1AK5514) | 254 | QVQLQQSGGGLVQAGGSLRLSCTASGSTFSFSNY |
| | | HMGWYRQAPGKQRERVASISSRDSTYYSDSVKG |
| | | RFTISRDTARNTVYLQMNSLEPEETAVYYCNARG |
| | | RATGRDYWGQGTQVTVSSGGSGGGGSGGGGSG |
| | | GGGSGGGGSGGDILLTQSPVILSVSPGERVSFSCR |
| | | ASQSIGTNIHWYQQRTNGSPRLLIKYASESISGIPS |
| | | RFSGSGSGTDFTLSINSVESEDIADYYCQQNNNWP |
| | | TTFGAGTKLELKRTVAAPSVFIFPPSDEQLKSGTA |
| | | SVVCLLNNFYPREAKVQWKVDNALQSGNSQESV |
| | | TEQDSKDSTYSLSSTLTLSKADYEKHKVYACEVT |
| | | HQGLSSPVTKSFNRGEC |
| LC (P1AK5515) | 255 | QVQLQEFGGGLVQAGEALRLSCVASKTIFNTMP |
| | | MGWYRQAPGKERELVATITSSGVVNSADSVKGR |
| | | FTISRDSAKRTAYLQMNNLKPEDTAVYYCAAMF |
| | | KPGIPEYWGRGTQVTVSSGGSGGGGSGGGGSGG |
| | | GGSGGGGSGGDILLTQSPVILSVSPGERVSFSCRA |
| | | SQSIGTNIHWYQQRTNGSPRLLIKYASESISGIPSRF |
| | | SGSGSGTDFTLSINSVESEDIADYYCQQNNNWPTT |
| | | FGAGTKLELKRTVAAPSVFIFPPSDEQLKSGTASV |
| | | VCLLNNFYPREAKVQWKVDNALQSGNSQESVTE |
| | | QDSKDSTYSLSSTLTLSKADYEKHKVYACEVTHQ |
| | | GLSSPVTKSFNRGEC |
| LC (P1AK5516) | 256 | QVQLQQSGGGLVQAGGSLRLSCTASGSTFSFSNY |
| | | HMGWYRQAPGKQRERVASISSRDSTYYSDSVKG |
| | | RFTISRDTARNTVYLQMNSLEPEETAVYYCNARG |
| | | RATGRDYWGQGTQVTVSSGGSGGGGSGGGGSG |
| | | GGGSGGGGSGGDIQMTQSPSSLSASVGDRVTITC |
| | | RASQGINNYLNWYQQKPGKAPKRLIYNTNNLQT |
| | | GVPSRFSGSGSGTEFTLTISSLQPEDFATYYCLQH |
| | | NSFPTFGQGTKLEIKRTVAAPSVFIFPPSDEQLKSG |
| | | TASVVCLLNNFYPREAKVQWKVDNALQSGNSQE |
| | | SVTEQDSKDSTYSLSSTLTLSKADYEKHKVYACE |
| | | VTHQGLSSPVTKSFNRGEC |
| LC (P1AK5517) | 257 | QVQLQEFGGGLVQAGEALRLSCVASKTIFNTMP |
| | | MGWYRQAPGKERELVATITSSGVVNSADSVKGR |
| | | FTISRDSAKRTAYLQMNNLKPEDTAVYYCAAMF |
| | | KPGIPEYWGRGTQVTVSSGGSGGGGSGGGGSGG |
| | | GGSGGGGSGGDIQMTQSPSSLSASVGDRVTITCR |
| | | ASQGINNYLNWYQQKPGKAPKRLIYNTNNLQTG |
| | | VPSRFSGSGSGTEFTLTISSLQPEDFATYYCLQHNS |
| | | FPTFGQGTKLEIKRTVAAPSVFIFPPSDEQLKSGTA |
| | | SVVCLLNNFYPREAKVQWKVDNALQSGNSQESV |
| | | TEQDSKDSTYSLSSTLTLSKADYEKHKVYACEVT |
| | | HQGLSSPVTKSFNRGEC |
| knob chain (P1AK5518) | 258 | QVQLQQSGGGLVQAGGSLRLSCTASGSTFSFSNY |
| | | HMGWYRQAPGKQRERVASISSRDSTYYSDSVKG |
| | | RFTISRDTARNTVYLQMNSLEPEETAVYYCNARG |
| | | RATGRDYWGQGTQVTVSSGGSGGGGSGGGGSG |
| | | GGGSGGGGSGGQVQLKQSGPGLVQPSQSLSITCT |
| | | VSGFSLTNYGVHWVRQSPGKGLEWLGVIWSGGN |
| | | TDYNTPFTSRLSINKDNSKSQVFFKMNSLQSNDT |
| | | AIYYCARALTYYDYEFAYWGQGTLVTVSAASTK |
| | | GPSVFPLAPSSKSTSGGTAALGCLVKDYFPEPVTV |
| | | SWNSGALTSGVHTFPAVLQSSGLYSLSSVVTVPSS |
| | | SLGTQTYICNVNHKPSNTKVDKKVEPKSCDKTHT |
| | | CPPCPAPEAAGGPSVFLFPPKPKDTLMISRTPEVTC |
| | | VVVDVSHEDPEVKFNWYVDGVEVHNAKTKPRE |
| | | EQYNSTYRVVSVLTVLHQDWLNGKEYKCKVSN |
| | | KALGAPIEKTISKAKGQPREPQVYTLPPCRDELTK |
| | | NQVSLWCLVKGFYPSDIAVEWESNGQPENNYKT |
| | | TPPVLDSDGSFFLYSKLTVDKSRWQQGNVFSCSV |
| | | MHEALHNHYTQKSLSLSPG |
| knob chain (P1AK5519) | 259 | QVQLQEFGGGLVQAGEALRLSCVASKTIFNTMP |
| | | MGWYRQAPGKERELVATITSSGVVNSADSVKGR |
| | | FTISRDSAKRTAYLQMNNLKPEDTAVYYCAAMF |
| | | KPGIPEYWGRGTQVTVSSGGSGGGGSGGGGSGG |
| | | GGSGGGGSGGQVQLKQSGPGLVQPSQSLSITCTV |
| | | SGFSLTNYGVHWVRQSPGKGLEWLGVIWSGGNT |
| | | DYNTPFTSRLSINKDNSKSQVFFKMNSLQSNDTAI |
| | | YYCARALTYYDYEFAYWGQGTLVTVSAASTKGP |
| | | SVFPLAPSSKSTSGGTAALGCLVKDYFPEPVTVS |
| | | WNSGALTSGVHTFPAVLQSSGLYSLSSVVTVPSSS |
| | | LGTQTYICNVNHKPSNTKVDKKVEPKSCDKTHTC |
| | | PPCPAPEAAGGPSVFLFPPKPKDTLMISRTPEVTC |
| | | VVVDVSHEDPEVKFNWYVDGVEVHNAKTKPRE |
| | | EQYNSTYRVVSVLTVLHQDWLNGKEYKCKVSN |
| | | KALGAPIEKTISKAKGQPREPQVYTLPPCRDELTK |
| | | NQVSLWCLVKGFYPSDIAVEWESNGQPENNYKT |
| | | TPPVLDSDGSFFLYSKLTVDKSRWQQGNVFSCSV |
| | | MHEALHNHYTQKSLSLSPG |
| knob chain (P1AK5520) | 260 | QVQLQQSGGGLVQAGGSLRLSCTASGSTFSFSNY |
| | | HMGWYRQAPGKQRERVASISSRDSTYYSDSVKG |
| | | RFTISRDTARNTVYLQMNSLEPEETAVYYCNARG |
| | | RATGRDYWGQGTQVTVSSGGSGGGGSGGGGSG |
| | | GGGSGGGGSGGQVQLVQSGAEVKKPGSSVKVSC |
| | | KASGFTFTDYKIHWVRQAPGQGLEWMGYFNPNS |
| | | GYSTYAQKFQGRVTITADKSTSTAYMELSSLRSE |
| | | DTAVYYCARLSPGGYYVMDAWGQGTTVTVSSA |
| | | STKGPSVFPLAPSSKSTSGGTAALGCLVKDYFPEP |
| | | VTVSWNSGALTSGVHTFPAVLQSSGLYSLSSVVT |
| | | VPSSSLGTQTYICNVNHKPSNTKVDKKVEPKSCD |
| | | KTHTCPPCPAPEAAGGPSVFLFPPKPKDTLMISRT |
| | | PEVTCVVVDVSHEDPEVKFNWYVDGVEVHNAK |
| | | TKPREEQYNSTYRVVSVLTVLHQDWLNGKEYKC |
| | | KVSNKALGAPIEKTISKAKGQPREPQVYTLPPCRD |
| | | ELTKNQVSLWCLVKGFYPSDIAVEWESNGQPEN |
| | | NYKTTPPVLDSDGSFFLYSKLTVDKSRWQQGNVF |
| | | SCSVMHEALHNHYTQKSLSLSPG |
| knob chain (P1AK5521) | 261 | QVQLQEFGGGLVQAGEALRLSCVASKTIFNTMP |
| | | MGWYRQAPGKERELVATITSSGVVNSADSVKGR |
| | | FTISRDSAKRTAYLQMNNLKPEDTAVYYCAAMF |
| | | KPGIPEYWGRGTQVTVSSGGSGGGGSGGGGSGG |
| | | GGSGGGGSGGQVQLVQSGAEVKKPGSSVKVSCK |
| | | ASGFTFTDYKIHWVRQAPGQGLEWMGYFNPNSG |
| | | YSTYAQKFQGRVTITADKSTSTAYMELSSLRSED |
| | | TAVYYCARLSPGGYYVMDAWGQGTTVTVSSAST |
| | | KGPSVFPLAPSSKSTSGGTAALGCLVKDYFPEPVT |
| | | VSWNSGALTSGVHTFPAVLQSSGLYSLSSVVTVP |
| | | SSSLGTQTYICNVNHKPSNTKVDKKVEPKSCDKT |
| | | HTCPPCPAPEAAGGPSVFLFPPKPKDTLMISRTPE |
| | | VTCVVVDVSHEDPEVKFNWYVDGVEVHNAKTK |
| | | PREEQYNSTYRVVSVLTVLHQDWLNGKEYKCKV |
| | | SNKALGAPIEKTISKAKGQPREPQVYTLPPCRDEL |
| | | TKNQVSLWCLVKGFYPSDIAVEWESNGQPENNY |
| | | KTTPPVLDSDGSFFLYSKLTVDKSRWQQGNVFSC |
| | | SVMHEALHNHYTQKSLSLSPG |
| (G4S)₁ peptide linker | 262 | GGGGS |
| (G4S)₃ peptide linker | 263 | GGGGSGGGGSGGGGS |
| (G4S)₅ peptide linker | 264 | GGGGSGGGGSGGGGSGGGGSGGGGS |
| LC (P1AL9238, | 265 | DIQMTQSPSSLSASVGDRVTITCRASQSISSYLNW |
| P1AL9239) | | YQQKPGKAPKLLIYTASSLQSGVPSRFSGSGSGTD |
| | | FTLTISSLQPEDFATYYCQQSYSTPLTFGGGTKVEI |
| | | KRTVAAPSVFIFPPSDRKLKSGTASVVCLLNNFYP |
| | | REAKVQWKVDNALQSGNSQESVTEQDSKDSTYS |
| | | LSSTLTLSKADYEKHKVYACEVTHQGLSSPVTKS |
| | | FNRGEC |
| LC2 P1AL9238 | 266 | QVQLVQSGAEVKKPGSSVKVSCKASGGTFSSYAI |
| | | SWVRQAPGQGLEWMGGIIPIFGTANYAQKFQGR |
| | | VTITADKSTSTAYMELSSLRSEDTAVYYCARDPG |
| | | PPYNWYWGAYDYWGQGTTVTVSSASVAAPSVFI |
| | | FPPSDEQLKSGTASVVCLLNNFYPREAKVQWKV |
| | | DNALQSGNSQESVTEQDSKDSTYSLSSTLTLSKA |
| | | DYEKHKVYACEVTHQGLSSPVTKSFNRGEC |
| knob chain P1AL9238 | 267 | DIQMTQSPSTLSASVGDRVTITCRASQSISSWLAW |
| | | YQQKPGKAPKLLIYDASSLESGVPSRFSGSGSGTE |
| | | FTLTISSLQPDDFATYYCQQYNSDWWTFGQGTKV |
| | | EIKSSASTKGPSVFPLAPSSKSTSGGTAALGCLVK |
| | | DYFPEPVTVSWNSGALTSGVHTFPAVLQSSGLYS |
| | | LSSVVTVPSSSLGTQTYICNVNHKPSNTKVDKKV |
| | | EPKSCGGGGSGGGGEVQLLESGGGLVQPGGSLRL |
| | | SCAASGFTFSSYAMSWVRQAPGKGLEWVSAITGS |
| | | GGSTYYADSVKGRFTISRDNSRNTLYLQMNSLRA |
| | | EDTAVYYCAKGEGYAGSSYFRASDIWGQGTMVT |
| | | VSSASTKGPSVFPLAPSSKSTSGGTAALGCLVEDY |
| | | FPEPVTVSWNSGALTSGVHTFPAVLQSSGLYSLSS |
| | | VVTVPSSSLGTQTYICNVNHKPSNTKVDEKVEPK |
| | | SCDKTHTCPPCPAPEAAGGPSVFLFPPKPKDTLMI |
| | | SRTPEVTCVVVDVSHEDPEVKFNWYVDGVEVHN |
| | | AKTKPREEQYNSTYRVVSVLTVLHQDWLNGKEY |
| | | KCKVSNKALGAPIEKTISKAKGQPREPQVYTLPPC |
| | | RDELTKNQVSLWCLVKGFYPSDIAVEWESNGQPE |
| | | NNYKTTPPVLDSDGSFFLYSKLTVDKSRWQQGN |
| | | VFSCSVMHEALHNHYTQKSLSLSPG |
| LC2 P1AL9239 | 268 | EVQLLESGGGLVQPGGSLRLSCAASGFTFSSYAM |
| | | SWVRQAPGKGLEWVSAISGSGGSTYYADSVKGR |
| | | FTISRDNSKNTLYLQMNSLRAEDTAVYYCAKVY |
| | | YYGFDYWGQGTLVTVSSASVAAPSVFIFPPSDEQ |
| | | LKSGTASVVCLLNNFYPREAKVQWKVDNALQSG |
| | | NSQESVTEQDSKDSTYSLSSTLTLSKADYEKHKV |
| | | YACEVTHQGLSSPVTKSFNRGEC |
| knob chain P1AL9239 | 269 | DIQMTQSPSTLSASVGDRVTITCRASQSISSWLAW |
| | | YQQKPGKAPKLLIYDASSLESGVPSRFSGSGSGTE |
| | | FTLTISSLQPDDFATYYCQQYASWYTFGQGTKVEI |
| | | KSSASTKGPSVFPLAPSSKSTSGGTAALGCLVKDY |
| | | FPEPVTVSWNSGALTSGVHTFPAVLQSSGLYSLSS |
| | | VVTVPSSSLGTQTYICNVNHKPSNTKVDKKVEPK |
| | | SCGGGGSGGGGEVQLLESGGGLVQPGGSLRLSCA |
| | | ASGFTFSSYAMSWVRQAPGKGLEWVSAITGSGGS |
| | | TYYADSVKGRFTISRDNSRNTLYLQMNSLRAEDT |
| | | AVYYCAKGEGYAGSSYFRASDIWGQGTMVTVSS |
| | | ASTKGPSVFPLAPSSKSTSGGTAALGCLVEDYFPE |
| | | PVTVSWNSGALTSGVHTFPAVLQSSGLYSLSSVV |
| | | TVPSSSLGTQTYICNVNHKPSNTKVDEKVEPKSC |
| | | DKTHTCPPCPAPEAAGGPSVFLFPPKPKDTLMISR |
| | | TPEVTCVVVDVSHEDPEVKFNWYVDGVEVHNA |
| | | KTKPREEQYNSTYRVVSVLTVLHQDWLNGKEYK |
| | | CKVSNKALGAPIEKTISKAKGQPREPQVYTLPPCR |
| | | DELTKNQVSLWCLVKGFYPSDIAVEWESNGQPE |
| | | NNYKTTPPVLDSDGSFFLYSKLTVDKSRWQQGN |
| | | VFSCSVMHEALHNHYTQKSLSLSPG |
| LC (P1AL9240, | 270 | EVQLLESGGGLVQPGGSLRLSCAASGFSFSSYTMS |
| P1AL9241) | | WVRQAPGKGLEWVATISGGGRDIYYPDSVKGRF |
| | | TISRDNSKNTLYLQMNSLRAEDTAVYYCVLLTGR |
| | | VYFALDSWGQGTLVTVSSASVAAPSVFIFPPSDEQ |
| | | LKSGTASVVCLLNNFYPREAKVQWKVDNALQSG |
| | | NSQESVTEQDSKDSTYSLSSTLTLSKADYEKHKV |
| | | YACEVTHQGLSSPVTKSFNRGEC |
| LC2 P1AL9240 | 271 | QIVLTQSPAIMSASPGEKVTMTCSGSSSVSFMYW |
| | | YQQRPGSSPRLLIYDTSNLASGVPVRFSGSGSGTS |
| | | YSLTISRMEAEDAATYYCQQWSTYPLTFGAGTKL |
| | | ELKRTVAAPSVFIFPPSDRKLKSGTASVVCLLNNF |
| | | YPREAKVQWKVDNALQSGNSQESVTEQDSKDST |
| | | YSLSSTLTLSKADYEKHKVYACEVTHQGLSSPVT |
| | | KSFNRGEC |
| knob chain P1AL9240 | 272 | QVQLKQSGPGLVQPSQSLSITCTVSGFSVTSYGVH |
| | | WIRQSPGKGLEWLGVIWSGGSTDYNAAFISRLTIS |
| | | KDNSKSQVFFKVNSLQPADTAIYYCARAGDYNY |
| | | DGFAYWGQGTLVTVSSASTKGPSVFPLAPSSKST |
| | | SGGTAALGCLVEDYFPEPVTVSWNSGALTSGVHT |
| | | FPAVLQSSGLYSLSSVVTVPSSSLGTQTYICNVNH |
| | | KPSNTKVDEKVEPKSCGGGGSGGGGDIVMTQSP |
| | | DSLAVSLGERATINCKASESVDTSDNSFIHWYQQ |
| | | KPGQSPKLLIYRSSTLESGVPDRFSGSGSGTDFTLT |
| | | ISSLQAEDVAVYYCQQNYDVPWTFGQGTKVEIKS |
| | | SASTKGPSVFPLAPSSKSTSGGTAALGCLVKDYFP |
| | | EPVTVSWNSGALTSGVHTFPAVLQSSGLYSLSSV |
| | | VTVPSSSLGTQTYICNVNHKPSNTKVDKKVEPKS |
| | | CDKTHTCPPCPAPEAAGGPSVFLFPPKPKDTLMIS |
| | | RTPEVTCVVVDVSHEDPEVKFNWYVDGVEVHNA |
| | | KTKPREEQYNSTYRVVSVLTVLHQDWLNGKEYK |
| | | CKVSNKALGAPIEKTISKAKGQPREPQVYTLPPCR |
| | | DELTKNQVSLWCLVKGFYPSDIAVEWESNGQPE |
| | | NNYKTTPPVLDSDGSFFLYSKLTVDKSRWQQGN |
| | | VFSCSVMHEALHNHYTQKSLSLSPG |
| LC2 P1AL9241 | 273 | DIVMTQSPLSLPVTPGEPASISCRSSQSLLHSNGYN |
| | | YLDWYLQKPGQSPQLLIYLGSNRASGVPDRFSGS |
| | | GSGTDFTLKISRVEAEDVGVYYCMQVLQPPPTFG |
| | | QGTKVEIKRTVAAPSVFIFPPSDRKLKSGTASVVC |
| | | LLNNFYPREAKVQWKVDNALQSGNSQESVTEQD |
| | | SKDSTYSLSSTLTLSKADYEKHKVYACEVTHQGL |
| | | SSPVTKSFNRGEC |
| knob chain P1AL9241 | 274 | QVQLVQSGAEVKKPGASVKVSCKASGYTFTSYY |
| | | MHWVRQAPGQGLEWMGIINPSGGSTSYAQKFQG |
| | | RVTMTRDTSTSTVYMELSSLRSEDTAVYYCARD |
| | | YSWWGDSYTGFDYWGQGTLVTVSSASTKGPSVF |
| | | PLAPSSKSTSGGTAALGCLVEDYFPEPVTVSWNS |
| | | GALTSGVHTFPAVLQSSGLYSLSSVVTVPSSSLGT |
| | | QTYICNVNHKPSNTKVDEKVEPKSCGGGGSGGG |
| | | GDIVMTQSPDSLAVSLGERATINCKASESVDTSDN |
| | | SFIHWYQQKPGQSPKLLIYRSSTLESGVPDRFSGS |
| | | GSGTDFTLTISSLQAEDVAVYYCQQNYDVPWTFG |
| | | QGTKVEIKSSASTKGPSVFPLAPSSKSTSGGTAAL |
| | | GCLVKDYFPEPVTVSWNSGALTSGVHTFPAVLQS |
| | | SGLYSLSSVVTVPSSSLGTQTYICNVNHKPSNTKV |
| | | DKKVEPKSCDKTHTCPPCPAPEAAGGPSVFLFPPK |
| | | PKDTLMISRTPEVTCVVVDVSHEDPEVKFNWYV |
| | | DGVEVHNAKTKPREEQYNSTYRVVSVLTVLHQD |
| | | WLNGKEYKCKVSNKALGAPIEKTISKAKGQPREP |
| | | QVYTLPPCRDELTKNQVSLWCLVKGFYPSDIAVE |
| | | WESNGQPENNYKTTPPVLDSDGSFFLYSKLTVDK |
| | | SRWQQGNVFSCSVMHEALHNHYTQKSLSLSPG |
| LC P1AL9310 | 275 | DIQMTQSPSSLSASVGDRVTITCRASQSISSYLNW |
| | | YQQKPGKAPKLLIYTASSLQSGVPSRFSGSGSGTD |
| | | FTLTISSLQPEDFATYYCQQSYSTPLTFGGGTKVEI |
| | | KGQPKAAPSVTLFPPSSEELQANKATLVCLISDFY |
| | | PGAVTVAWKADSSPVKAGVETTTPSKQSNNKYA |
| | | ASSYLSLTPEQWKSHRSYSCQVTHEGSTVEKTVA |
| | | PTECS |
| knob chain P1AL9310 | 276 | EVQLLESGGGLVQPGGSLRLSCAASGFTFSSYAM |
| | | SWVRQAPGKGLEWVSAISGSGGSTYYADSVKGR |
| | | FTISRDNSKNTLYLQMNSLRAEDTAVYYCARLLY |
| | | YGFDYWGQGTLVTVSSGGSGGGGSGGGGSGGG |
| | | GSGGDIQMTQSPSTLSASVGDRVTITCRASQSISS |
| | | WLAWYQQKPGKAPKLLIYDASSLESGVPSRFSGS |
| | | GSGTEFTLTISSLQPDDFATYYCQQYSSYYTFGQG |
| | | TKVEIKGGGGSGGGGEVQLLESGGGLVQPGGSLR |
| | | LSCAASGFTFSSYAMSWVRQAPGKGLEWVSAITG |
| | | SGGSTYYADSVKGRFTISRDNSRNTLYLQMNSLR |
| | | AEDTAVYYCAKGEGYAGSSYFRASDIWGQGTMV |
| | | TVSSASTKGPSVFPLAPSSKSTSGGTAALGCLVKD |
| | | YFPEPVTVSWNSGALTSGVHTFPAVLQSSGLYSLS |
| | | SVVTVPSSSLGTQTYICNVNHKPSNTKVDKKVEP |
| | | KSCDKTHTCPPCPAPEAAGGPSVFLFPPKPKDTLM |
| | | ISRTPEVTCVVVDVSHEDPEVKFNWYVDGVEVH |
| | | NAKTKPREEQYNSTYRVVSVLTVLHQDWLNGKE |
| | | YKCKVSNKALGAPIEKTISKAKGQPREPQVYTLPP |
| | | CRDELTKNQVSLWCLVKGFYPSDIAVEWESNGQ |
| | | PENNYKTTPPVLDSDGSFFLYSKLTVDKSRWQQG |
| | | NVFSCSVMHEALHNHYTQKSLSLSPG |
| knob chain P1AL9311 | 277 | EVQLLESGGGLVQPGGSLRLSCAASGFSFSSYTMS |
| | | WVRQAPGKGLEWVATISGGGRDIYYPDSVKGRF |
| | | TISRDNSKNTLYLQMNSLRAEDTAVYYCVLLTGR |
| | | VYFALDSWGQGTLVTVSSASTKGPSVFPLAPSSK |
| | | STSGGTAALGCLVKDYFPEPVTVSWNSGALTSGV |
| | | HTFPAVLQSSGLYSLSSVVTVPSSSLGTQTYICNV |
| | | NHKPSNTKVDKKVEPKSCDKTHTCPPCPAPEAAG |
| | | GPSVFLFPPKPKDTLMISRTPEVTCVVVDVSHEDP |
| | | EVKFNWYVDGVEVHNAKTKPREEQYNSTYRVVS |
| | | VLTVLHQDWLNGKEYKCKVSNKALGAPIEKTISK |
| | | AKGQPREPQVYTLPPCRDELTKNQVSLWCLVKG |
| | | FYPSDIAVEWESNGQPENNYKTTPPVLDSDGSFFL |
| | | YSKLTVDKSRWQQGNVFSCSVMHEALHNHYTQ |
| | | KSLSLSPG |
| LC P1AL9311 | 278 | QVQLVQSGAEVKKPGASVKVSCKASGYTFTSYY |
| | | MHWVRQAPGQGLEWMGIINPSGGSTSYAQKFQG |
| | | RVTMTRDTSTSTVYMELSSLRSEDTAVYYCARD |
| | | YSWWGDSYTGFDYWGQGTLVTVSSGGSGGGGS |
| | | GGGGSGGGGSGGDIVMTQSPLSLPVTPGEPASISC |
| | | RSSQSLLHSNGYNYLDWYLQKPGQSPQLLIYLGS |
| | | NRASGVPDRFSGSGSGTDFTLKISRVEAEDVGVY |
| | | YCMQVLQPPPTFGQGTKVEIKGGGGSGGGGDIV |
| | | MTQSPDSLAVSLGERATINCKASESVDTSDNSFIH |
| | | WYQQKPGQSPKLLIYRSSTLESGVPDRFSGSGSGT |
| | | DFTLTISSLQAEDVAVYYCQQNYDVPWTFGQGT |
| | | KVEIKGQPKAAPSVTLFPPSSEELQANKATLVCLI |
| | | SDFYPGAVTVAWKADSSPVKAGVETTTPSKQSN |
| | | NKYAASSYLSLTPEQWKSHRSYSCQVTHEGSTVE |
| | | KTVAPTECS |
| (G2SG2)₅ peptide linker | 279 | GGSGG GGSGG GGSGG GGSGG GGSGG |
| G4S_G4 peptide linker | 280 | GGGGS GGGG |
| (G2SG2)4 peptide linker | 281 | GGSGG GGSGG GGSGG GGSGG |
| Fab IL-2Rγ binder | 282 | QVQLVQSGAEVKKPGSSVKVSCKASGGTFSSYAI |
| FV018863 VH | | SWVRQAPGQGLEWMGGIIPIFGTANYAQKFQGR |
| | | VTITADKSTSTAYMELSSLRSEDTAVYYCARDPG |
| | | PPYNWYWGAYDYWGQGTTVTVSS |
| Fab IL-2Rγ binder | 283 | DIQMTQSPSTLSASVGDRVTITCRASQSISSWLAW |
| FV018863 VL | | YQQKPGKAPKLLIYDASSLESGVPSRFSGSGSGTE |
| | | FTLTISSLQPDDFATYYCQQYNSDWWTFGQGTKV |
| | | EIK |
| Fab IL-2Rγ binder | 284 | EVQLLESGGGLVQPGGSLRLSCAASGFTFSSYAM |
| FV018864 VH | | SWVRQAPGKGLEWVSAISGSGGSTYYADSVKGR |
| | | FTISRDNSKNTLYLQMNSLRAEDTAVYYCAKVY |
| | | YYGFDYWGQGTLVTVSS |
| Fab IL-2Rγ binder | 285 | DIQMTQSPSTLSASVGDRVTITCRASQSISSWLAW |
| FV018864 VL | | YQQKPGKAPKLLIYDASSLESGVPSRFSGSGSGTE |
| | | FTLTISSLQPDDFATYYCQQYASWYTFGQGTKVEI |
| | | K |
| Fab IL-2Rβ binder | 286 | QVQLKQSGPGLVQPSQSLSITCTVSGFSVTSYGVH |
| FV002203 VH | | WIRQSPGKGLEWLGVIWSGGSTDYNAAFISRLTIS |
| | | KDNSKSQVFFKVNSLQPADTAIYYCARAGDYNY |
| | | DGFAYWGQGTLVTVSS |
| Fab IL-2Rβ binder | 287 | QIVLTQSPAIMSASPGEKVTMTCSGSSSVSFMYW |
| FV002203 VL | | YQQRPGSSPRLLIYDTSNLASGVPVRFSGSGSGTS |
| | | YSLTISRMEAEDAATYYCQQWSTYPLTFGAGTKL |
| | | ELK |
| Fab IL-2Rβ binder | 288 | QVQLVQSGAEVKKPGASVKVSCKASGYTFTSYY |
| FV018866 VH | | MHWVRQAPGQGLEWMGIINPSGGSTSYAQKFQG |
| | | RVTMTRDTSTSTVYMELSSLRSEDTAVYYCARD |
| | | YSWWGDSYTGFDYWGQGTLVTVSS |
| Fab IL-2Rβ binder | 289 | DIVMTQSPLSLPVTPGEPASISCRSSQSLLHSNGYN |
| FV018866 VL | | YLDWYLQKPGQSPQLLIYLGSNRASGVPDRFSGS |
| | | GSGTDFTLKISRVEAEDVGVYYCMQVLQPPPTFG |
| | | QGTKVEIK |
| scFv IL-2Rγ binder | 290 | EVQLLESGGGLVQPGGSLRLSCAASGFTFSSYAM |
| FV018865 VH | | SWVRQAPGKGLEWVSAISGSGGSTYYADSVKGR |
| | | FTISRDNSKNTLYLQMNSLRAEDTAVYYCARLLY |
| | | YGFDYWGQGTLVTVSS |
| scFv IL-2Rγ binder | 291 | DIQMTQSPSTLSASVGDRVTITCRASQSISSWLAW |
| FV018865 VL | | YQQKPGKAPKLLIYDASSLESGVPSRFSGSGSGTE |
| | | FTLTISSLQPDDFATYYCQQYSSYYTFGQGTKVEI |
| | | K |
| scFv IL-2Rβ binder | 292 | DIVMTQSPLSLPVTPGEPASISCRSSQSLLHSNGYN |
| FV018866 VL | | YLDWYLQKPGQSPQLLIYLGSNRASGVPDRFSGS |
| | | GSGTDFTLKISRVEAEDVGVYYCMQVLQPPPTFG |
| | | QGTKVEIK |
| scFv IL-2Rβ binder | 293 | QVQLVQSGAEVKKPGASVKVSCKASGYTFTSYY |
| FV018866 VH | | MHWVRQAPGQGLEWMGIINPSGGSTSYAQKFQG |
| | | RVTMTRDTSTSTVYMELSSLRSEDTAVYYCARD |
| | | YSWWGDSYTGFDYWGQGTLVTVSS |

1. A pair of antigen binding molecules that specifically bind to a target antigen, comprising
a) a first antigen binding molecule comprising i) a first target-binding domain, ii) a first cytokine receptor-binding domain, and iii) a Fc domain; and
b) a second antigen binding molecule comprising i) a second target-binding domain, ii) a second cytokine receptor-binding domain, and iii) a Fc domain;
wherein the first target-binding domain is capable of binding a first epitope on the target antigen and the second target-binding domain is capable of binding a second epitope on the target antigen, wherein the first and second target-binding domains do not compete for binding on the target antigen; and
wherein the first cytokine receptor-binding domain is capable of binding a first cytokine receptor subunit and the second cytokine receptor-binding domain is capable of binding a second cytokine receptor subunit.

2. The pair of antigen binding molecules according to claim 1, wherein the first and second target-binding domains are antibody fragments, such as Fv, Fab, scFv, scFab molecules or single domain antibodies.

3. The pair of antigen binding molecules according to claim 1, wherein the first and second target-binding domains are Fab molecules.

4. The pair of antigen binding molecules according to claim 3, wherein the first target-binding domain comprises a heavy chain variable domain (VH₁), a light chain variable domain (VL₁), a heavy chain constant domain (CH1₁) and a light chain constant domain (CL₁), and wherein the second target-binding domain comprises a heavy chain variable domain (VH₂), a light chain variable domain (VL₂), a heavy chain constant domain (CH1₂) and a light chain constant domain (CL₂).

5. The pair of antigen binding molecules according to claim 1, wherein the first and/or second target-binding domain is a cross-Fab molecule.

6. The pair of antigen binding molecules according to claim 1, wherein the first and second target-binding domains specifically bind to a tumor-associated antigen or a T-cell antigen.

7. The pair of antigen binding molecules according to claim 1, wherein the first and second target-binding domains specifically bind to FAP, PD-1, Her2, Her3, LAG-3 or EGFR.

8. The pair of antigen binding molecules according to claim 1, wherein
a) the first target-binding domain comprises a VH₁ of SEQ ID NO: 20 and a VL₁ of SEQ ID NO: 21 and the second target-binding domain comprises a VH₂ of SEQ ID NO: 22 and a VL₂ of SEQ ID NO: 23, or
b) the first target-binding domain comprises a VH₁ of SEQ ID NO: 22 and a VL₁ of SEQ ID NO: 23 and the second target-binding domain comprises a VH₂ of SEQ ID NO: 20 and a VL₂ of SEQ ID NO: 21, or
c) the first target-binding domain comprises a VH₁ of SEQ ID NO: 76 and a VL₁ of SEQ ID NO: 77 and the second target-binding domain comprises a VH₂ of SEQ ID NO: 78 and a VL₂ of SEQ ID NO: 79, or
d) the first target-binding domain comprises a VH₁ of SEQ ID NO: 78 and a VL₁ of SEQ ID NO: 79 and the second target-binding domain comprises a VH₂ of SEQ ID NO: 76 and a VL₂ of SEQ ID NO: 77; or
e) the first target-binding domain comprises a VH₁ of SEQ ID NO: 93 and a VL₁ of SEQ ID NO: 94 and the second target-binding domain comprises a VH₂ of SEQ ID NO: 78 and a VL₂ of SEQ ID NO: 79; or
f) the first target-binding domain comprises a VH₁ of SEQ ID NO: 78 and a VL₁ of SEQ ID NO: 79 and the second target-binding domains comprises a VH₂ of SEQ ID NO: 93 and a VL₂ of SEQ ID NO: 94, or
g) the first target-binding domain comprises a VH₁ of SEQ ID NO: 130 and a VL₁ of SEQ ID NO: 131 and the second target-binding domain comprises a VH₂ of SEQ ID NO: 132 and a VL₂ of SEQ ID NO: 133; or
h) the first target-binding domain comprises a VH₁ of SEQ ID NO: 132 and a VL₁ of SEQ ID NO: 133 and the second target-binding domains comprises a VH₂ of SEQ ID NO: 130 and a VL₂ of SEQ ID NO: 131; or
i) the first target-binding domain comprises a VH₁ of SEQ ID NO: 140 and a VL₁ of SEQ ID NO: 141 and the second target-binding domain comprises a VH₂ of SEQ ID NO: 142 and a VL₂ of SEQ ID NO: 143; or
j) the first target-binding domain comprises a VH₁ of SEQ ID NO: 142 and a VL₁ of SEQ ID NO: 143 and the second target-binding domains comprises a VH₂ of SEQ ID NO: 140 and a VL₂ of SEQ ID NO: 141; or
k) the first target-binding domain comprises a VH₁ of SEQ ID NO: 114 and a VL₁ of SEQ ID NO: 115 and the second target-binding domain comprises a VH₂ of SEQ ID NO: 116 and a VL₂ of SEQ ID NO: 117; or
l) the first target-binding domain comprises a VH₁ of SEQ ID NO: 116 and a VL₁ of SEQ ID NO: 117 and the second target-binding domains comprises a VH₂ of SEQ ID NO: 114 and a VL₂ of SEQ ID NO: 115; or
m) the first target-binding domain comprises a VH₁ of SEQ ID NO: 150 and a VL₁ of SEQ ID NO: 151 and the second target-binding domain comprises a VH₂ of SEQ ID NO: 152 and a VL₂ of SEQ ID NO: 153; or
n) the first target-binding domain comprises a VH₁ of SEQ ID NO: 152 and a VL₁ of SEQ ID NO: 153 and the second target-binding domains comprises a VH₂ of SEQ ID NO: 150 and a VL₂ of SEQ ID NO: 151.

9. The pair of antigen binding molecules according to claim 1, wherein both the first and second cytokine receptor subunits are subunits of the IFNγ receptor complex or, IL-2 receptor complex or IL-7 receptor complex.

10. The pair of antigen binding molecules according to claim 1, wherein
a) the first cytokine receptor-binding domain is capable of binding IFNγR1 and the second cytokine receptor-binding domain is capable of binding IFNγR2, or
b) the first cytokine receptor-binding domain is capable of binding IFNγR2 and the second cytokine receptor-binding domain is capable of binding IFNγR1; or
c) the first cytokine receptor-binding domain is capable of binding IL-2Rβ and the second cytokine receptor-binding domain is capable of binding IL-2Rγ, or
d) the first cytokine receptor-binding domain is capable of binding IL-2Rγ and the second cytokine receptor-binding domain is capable of binding IL-2Rβ; or
e) the first cytokine receptor-binding domain is capable of binding IL-2Rγ and the second cytokine receptor-binding domain is capable of binding IL-7Rα; or
f) the first cytokine receptor-binding domain is capable of binding IL-7Rα and the second cytokine receptor-binding domain is capable of binding IL-2Rγ.

11. The pair of antigen binding molecules according to claim 1, wherein the first and second cytokine receptor-binding domains are antibody fragments, such as Fv, Fab, scFv, scFab or single domain antibodies.

12. The pair of antigen binding molecules according to claim 1, wherein the first and second cytokine receptor-binding domains are VHH domains.

13. The pair of antigen binding molecules according to claim 1, wherein
a) the first cytokine receptor-binding domain comprises an amino acid sequence selected from SEQ ID NO: 1, SEQ ID NO: 2, SEQ ID NO: 3 and SEQ ID NO: 5 and the second cytokine receptor-binding domain comprises an amino acid sequence selected from SEQ ID NO: 6, SEQ ID NO: 7, SEQ ID NO: 8, SEQ ID NO: 9 and SEQ ID NO: 10, or
b) the first cytokine receptor-binding domain comprises an amino acid sequence selected from SEQ ID NO: 6, SEQ ID NO: 7, SEQ ID NO: 8, SEQ ID NO: 9 and SEQ ID NO: 10 and the second cytokine receptor-binding domain comprises an amino acid sequence selected from SEQ ID NO: 1, SEQ ID NO: 2, SEQ ID NO: 3 and SEQ ID NO: 5; or
c) the first cytokine receptor-binding domain comprises an amino acid sequence selected from SEQ ID NO: 56, SEQ ID NO: 57, SEQ ID NO: 58 SEQ ID NO: 60, and the second cytokine receptor-binding domain comprises an amino acid sequence selected from SEQ ID NO: 62, SEQ ID NO: 63, SEQ ID NO: 64 and SEQ ID NO: 65; or
d) the first cytokine receptor-binding domain comprises an amino acid sequence selected from SEQ ID NO: 62, SEQ ID NO: 63, SEQ ID NO: 64 and SEQ ID NO: 65 and the second cytokine receptor-binding domain comprises an amino acid sequence selected from SEQ ID NO: 56, SEQ ID NO: 57, SEQ ID NO: 58 SEQ ID NO: 60; or
e) the first cytokine receptor-binding domain comprises an amino acid sequence selected from SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, and the second cytokine receptor-binding domain comprises an amino acid sequence selected from SEQ ID NO: 61, SEQ ID NO: 62, SEQ ID NO: 63, SEQ ID NO: 64, SEQ ID NO: 65; or
f) the first cytokine receptor-binding domain comprises an amino acid sequence selected from SEQ ID NO: 61, SEQ ID NO: 62, SEQ ID NO: 63, SEQ ID NO: 64, SEQ ID NO: 65, and the second cytokine receptor-binding domain comprises an amino acid sequence selected from SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192.

14. The pair of antigen binding molecules according to claim 1, wherein
a) the first cytokine receptor-binding domain comprises the sequence of SEQ ID NO: 3 and the second cytokine receptor-binding domain comprises the sequence of SEQ ID NO: 7, or
b) the first cytokine receptor-binding domain comprises the sequence of SEQ ID NO: 7 and the second cytokine receptor-binding domain comprises the sequence of SEQ ID NO: 3; or
c) the first cytokine receptor-binding domain comprises the sequence of SEQ ID NO: 60 and the second cytokine receptor-binding domain comprises the sequence of SEQ ID NO: 62, or
d) the first cytokine receptor-binding domain comprises the sequence of SEQ ID NO: 62 and the second cytokine receptor-binding domain comprises the sequence of SEQ ID NO: 60; or
e) the first cytokine receptor-binding domain comprises the sequence of SEQ ID NO: 186, and the second cytokine receptor-binding domain comprises the sequence of SEQ ID NO: 62, or
f) the first cytokine receptor-binding domain comprises the sequence of SEQ ID NO: 62, and the second cytokine receptor-binding domain comprises the sequence of SEQ ID NO: 186.

15. The pair of antigen binding molecules according to claim 1, wherein the Fc domains of the first and second antigen binding molecules comprise a first Fc domain subunit and a second Fc domain subunit.

16. The pair of antigen binding molecules according to claim 1, wherein the Fc domains of the first and second antigen binding molecules are IgG, particularly an IgG₁, Fc domains.

17. The pair of antigen binding molecules according to claim 1, wherein the Fc domains of the first and second antigen binding molecules are human Fc domains.

18. The pair of antigen binding molecules according to claim 1, wherein the Fc domains of the first and second antigen binding molecules comprise a modification promoting the association of the first and the second subunit of the Fc domains.

19. The pair of antigen binding molecules according to claim 1, wherein the Fc domains of the first and second antigen binding molecules comprise one or more amino acid substitution that reduces binding to an Fc receptor and/or effector function.

20. The pair of antigen binding molecules according to claim 1, wherein the cytokine receptor-binding domains are fused via peptide linkers to their respective fusion points.

21. The pair of antigen binding molecules according to claim 20, wherein the peptide linkers comprise an amino acid sequence selected from SEQ ID NO: 262, SEQ ID NO: 263, SEQ ID NO: 264, SEQ ID NO: 279, SEQ ID NO: 280 or SEQ ID NO: 281.

22. The pair of antigen binding molecules according to claim 1, wherein the first cytokine receptor-binding domain is fused at its C-terminus to the N-terminus of VH₁ or VL₁ of the first target-binding domain and the second cytokine receptor-binding domain is fused at its C-terminus to the N-terminus of VH₂ or VL₂ of the second target-binding domain.

23. (canceled)

24. The pair of antigen binding molecules according to claim 1, wherein
a) the first antigen binding molecule comprises a first polypeptide comprising a first Fc domain subunit, a second polypeptide comprising in order from the N-terminus to C-terminus the first cytokine receptor-binding domain, VH₁, CH1₁ and a second Fc domain subunit, and a third polypeptide comprising in order from the N-terminus to C-terminus VL₁ and CL₁, and the second antigen binding molecule comprises a first polypeptide comprising a first Fc domain subunit, a second polypeptide comprising in order from the N-terminus to C-terminus the second cytokine receptor-binding domain, VH₂, CH1₂ and a second Fc domain subunit, and a third polypeptide comprising in order from the N-terminus to C-terminus VL₂ and CL₂; or
b) the first antigen binding molecule comprises a first polypeptide comprising a first Fc domain subunit, a second polypeptide comprising in order from the N-terminus to C-terminus VH₁, CH1₁ and a second Fc domain subunit, and a third polypeptide comprising in order from the N-terminus to C-terminus the first cytokine receptor-binding domain, VL₁ and CL₁, and the second antigen binding molecule comprises a first polypeptide comprising a first Fc domain subunit, a second polypeptide comprising in order from the N-terminus to C-terminus VH₂, CH1₂ and a second Fc domain subunit, a third polypeptide comprising in order from the N-terminus to C-terminus the second cytokine receptor-binding domain, VL₂ and CL₂; or
c) the first antigen binding molecule comprises a first polypeptide comprising a first Fc domain subunit, a second polypeptide comprising in order from the N-terminus to C-terminus the first cytokine receptor-binding domain, VH₁, CH1₁ and a second Fc domain subunit, and a third polypeptide comprising in order from the N-terminus to C-terminus VL₁ and CL₁, and the second antigen binding molecule comprises a first polypeptide comprising a first Fc domain subunit, a second polypeptide comprising in order from the N-terminus to C-terminus VH₂, CH1₂ and a second Fc domain subunit, a third polypeptide comprising in order from the N-terminus to C-terminus the second cytokine receptor-binding domain, VL₂ and CL₂; or
d) the first antigen binding molecule comprises a first polypeptide comprising a first Fc domain subunit, a second polypeptide comprising in order from the N-terminus to C-terminus the first cytokine receptor-binding domain, VL₁, CH1₁ and a second Fc domain subunit, and a third polypeptide comprising in order from the N-terminus to C-terminus VH₁ and CL₁, and the second antigen binding molecule comprises a first polypeptide comprising a first Fc domain subunit, a second polypeptide comprising in order from the N-terminus to C-terminus the second cytokine receptor-binding domain, VH₂, CH1₂ and a second Fc domain subunit, and a third polypeptide comprising in order from the N-terminus to C-terminus VL₂ and CL₂; or
e) the first antigen binding molecule comprises a first polypeptide comprising a first Fc domain subunit, a second polypeptide comprising in order from the N-terminus to C-terminus VL₁, CH1₁ and a second Fc domain subunit, and a third polypeptide comprising in order from the N-terminus to C-terminus the first cytokine receptor-binding domain, VH₁ and CL₁, and the second antigen binding molecule comprises a first polypeptide comprising a first Fc domain subunit, a second polypeptide comprising in order from the N-terminus to C-terminus VH₂, CH1₂ and a second Fc domain subunit, a third polypeptide comprising in order from the N-terminus to C-terminus the second cytokine receptor-binding domain, VL₂ and CL₂; or
f) the first antigen binding molecule comprises a first polypeptide comprising a first Fc domain subunit, a second polypeptide comprising in order from the N-terminus to C-terminus the first cytokine receptor-binding domain, VL₁, CH1₁ and a second Fc domain subunit, and a third polypeptide comprising in order from the N-terminus to C-terminus VH₁ and CL₁, and the second antigen binding molecule comprises a first polypeptide comprising a first Fc domain subunit, a second polypeptide comprising in order from the N-terminus to C-terminus VH₂, CH1₂ and a second Fc domain subunit, and a third polypeptide comprising in order from the N-terminus to C-terminus the second cytokine receptor-binding domain, VL₂ and CL₂; or
g) the first antigen binding molecule comprising a first polypeptide comprising a first Fc domain subunit, a second polypeptide comprising in order from the N-terminus to C-terminus a VL₁, CH1₁ and a second Fc domain subunit, and a third polypeptide comprising in order from the N-terminus to C-terminus a first cytokine receptor-binding domain, VH₁ and CL₁, and the second antigen binding molecule comprises a first polypeptide comprising a first Fc domain subunit, a second polypeptide comprising in order from the N-terminus to C-terminus a second cytokine receptor-binding domain, VH₂, CH1₂ and a second Fc domain subunit, and a third polypeptide comprising in order from the N-terminus to C-terminus VL₂ and CL₂; wherein VH₁, VL₁, CH1₁ and CL₁ form the first target-binding domain and VH₂, VL₂, CH1₂, and CL₂, form the second target-binding domain.

25. The pair of antigen binding molecules according to claim 1, wherein the first and the second target-binding domains specifically bind to FAP, and the first and second cytokine receptor subunits are subunits of the IFNγ receptor complex.

26. The pair of antigen binding molecules according to claim 1, wherein
a) the first antigen binding molecule comprises a first polypeptide comprising an amino acid sequence of SEQ ID NO: 28, a second polypeptide comprising an amino acid sequence of SEQ ID NO: 27 and a third polypeptide comprising an amino acid sequence of SEQ ID NO: 25, and the second antigen binding molecule comprises a first polypeptide comprising an amino acid sequence of SEQ ID NO: 28, a second polypeptide comprising an amino acid sequence of SEQ ID NO: 30 and a third polypeptide comprising an amino acid sequence of SEQ ID NO: 29; or
b) the first antigen binding molecule comprises a first polypeptide comprising an amino acid sequence of SEQ ID NO: 28, a second polypeptide comprising an amino acid sequence of SEQ ID NO: 38 and a third polypeptide comprising an amino acid sequence of SEQ ID NO: 25, and the second antigen binding molecule comprises a first polypeptide comprising an amino acid sequence of SEQ ID NO: 28, a second polypeptide comprising an amino acid sequence of SEQ ID NO: 37 and a third polypeptide comprising an amino acid sequence of SEQ ID NO: 29; or
c) the first antigen binding molecule comprises a first polypeptide comprising an amino acid sequence of SEQ ID NO: 28, a second polypeptide comprising an amino acid sequence of SEQ ID NO: 32 and a third polypeptide comprising an amino acid sequence of SEQ ID NO: 31, and the second antigen binding molecule comprises a first polypeptide comprising an amino acid sequence of SEQ ID NO: 28, a second polypeptide comprising an amino acid sequence of SEQ ID NO: 34 and a third polypeptide comprising an amino acid sequence of SEQ ID NO: 33; or
d) the first antigen binding molecule comprises a first polypeptide comprising an amino acid sequence of SEQ ID NO: 28, a second polypeptide comprising an amino acid sequence of SEQ ID NO: 32 and a third polypeptide comprising an amino acid sequence of SEQ ID NO: 35, and the second antigen binding molecule comprises a first polypeptide comprising an amino acid sequence of SEQ ID NO: 28, a second polypeptide comprising an amino acid sequence of SEQ ID NO: 34 and a third polypeptide comprising an amino acid sequence of SEQ ID NO: 36; or
e) the first antigen binding molecule comprises a first polypeptide comprising an amino acid sequence of SEQ ID NO: 28, a second polypeptide comprising an amino acid sequence of SEQ ID NO: 27 and a third polypeptide comprising an amino acid sequence of SEQ ID NO: 25, and the second antigen binding molecule comprises a first polypeptide comprising an amino acid sequence of SEQ ID NO: 28, a second polypeptide comprising an amino acid sequence of SEQ ID NO: 34 and a third polypeptide comprising an amino acid sequence of SEQ ID NO: 33; or
f) the first antigen binding molecule comprises a first polypeptide comprising an amino acid sequence of SEQ ID NO: 28, a second polypeptide comprising an amino acid sequence of SEQ ID NO: 30 and a third polypeptide comprising an amino acid sequence of SEQ ID NO: 29, and the second antigen binding molecule comprises a first polypeptide comprising an amino acid sequence of SEQ ID NO: 28, a second polypeptide comprising an amino acid sequence of SEQ ID NO: 32 and a third polypeptide comprising an amino acid sequence of SEQ ID NO: 31; or
g) the first antigen binding molecule comprising a first polypeptide comprises an amino acid sequence of SEQ ID NO: 28, a second polypeptide comprising an amino acid sequence of SEQ ID NO: 38 and a third polypeptide comprising an amino acid sequence of SEQ ID NO: 25, and the second antigen binding molecule comprises a first polypeptide comprising an amino acid sequence of SEQ ID NO: 28, a second polypeptide comprising an amino acid sequence of SEQ ID NO: 34 and a third polypeptide comprising an amino acid sequence of SEQ ID NO: 36; or
h) the first antigen binding molecule comprises a first polypeptide comprising an amino acid sequence of SEQ ID NO: 28, a second polypeptide comprising an amino acid sequence of SEQ ID NO: 37 and a third polypeptide comprising an amino acid sequence of SEQ ID NO: 29, and the second antigen binding molecule comprises a first polypeptide comprising an amino acid sequence of SEQ ID NO: 28, a second polypeptide comprising an amino acid sequence of SEQ ID NO: 32 and a third polypeptide comprising an amino acid sequence of SEQ ID NO: 35.

27. The pair of antigen binding molecules according to claim 1, wherein the first and the second target-binding domains specifically bind to EGFR, and the first and second cytokine receptor subunits are subunits of the IFNγ receptor complex.

28. The pair of antigen binding molecules according to claim 1, wherein
a) the first antigen binding molecule comprises a first polypeptide comprising an amino acid sequence of SEQ ID NO: 106, a second polypeptide comprising an amino acid sequence of SEQ ID NO: 260 and a third polypeptide comprising an amino acid sequence of SEQ ID NO: 124, and the second antigen binding molecule comprises a first polypeptide comprising an amino acid sequence of SEQ ID NO: 106, a second polypeptide comprising an amino acid sequence of SEQ ID NO: 259 and a third polypeptide comprising an amino acid sequence of SEQ ID NO: 127; or
b) the first antigen binding molecule comprises a first polypeptide comprising an amino acid sequence of SEQ ID NO: 106, a second polypeptide comprising an amino acid sequence of SEQ ID NO: 119 and a third polypeptide comprising an amino acid sequence of SEQ ID NO: 256, and the second antigen binding molecule comprises a first polypeptide comprising an amino acid sequence of SEQ ID NO: 106, a second polypeptide comprising an amino acid sequence of SEQ ID NO: 122 and a third polypeptide comprising an amino acid sequence of SEQ ID NO: 255; or
c) the first antigen binding molecule comprises a first polypeptide comprising an amino acid sequence of SEQ ID NO: 106, a second polypeptide comprising an amino acid sequence of SEQ ID NO: 261 and a third polypeptide comprising an amino acid sequence of SEQ ID NO: 124, and the second antigen binding molecule comprises a first polypeptide comprising an amino acid sequence of SEQ ID NO: 106, a second polypeptide comprising an amino acid sequence of SEQ ID NO: 258 and a third polypeptide comprising an amino acid sequence of SEQ ID NO: 127; or
d) the first antigen binding molecule comprises a first polypeptide comprising an amino acid sequence of SEQ ID NO: 106, a second polypeptide comprising an amino acid sequence of SEQ ID NO: 119 and a third polypeptide comprising an amino acid sequence of SEQ ID NO: 257, and the second antigen binding molecule comprises a first polypeptide comprising an amino acid sequence of SEQ ID NO: 106, a second polypeptide comprising an amino acid sequence of SEQ ID NO: 112 and a third polypeptide comprising an amino acid sequence of SEQ ID NO: 254; or
e) the first antigen binding molecule comprises a first polypeptide comprising an amino acid sequence of SEQ ID NO: 106, a second polypeptide comprising an amino acid sequence of SEQ ID NO: 260 and a third polypeptide comprising an amino acid sequence of SEQ ID NO: 124, and the second antigen binding molecule comprises a first polypeptide comprising an amino acid sequence of SEQ ID NO: 106, a second polypeptide comprising an amino acid sequence of SEQ ID NO: 112 and a third polypeptide comprising an amino acid sequence of SEQ ID NO: 255; or
f) the first antigen binding molecule comprises a first polypeptide comprising an amino acid sequence of SEQ ID NO: 106, a second polypeptide comprising an amino acid sequence of SEQ ID NO: 119 and a third polypeptide comprising an amino acid sequence of SEQ ID NO: 256, and the second antigen binding molecule comprises a first polypeptide comprising an amino acid sequence of SEQ ID NO: 106, a second polypeptide comprising an amino acid sequence of SEQ ID NO: 259 and a third polypeptide comprising an amino acid sequence of SEQ ID NO: 127; or
g) the first antigen binding molecule comprises a first polypeptide comprising an amino acid sequence of SEQ ID NO: 106, a second polypeptide comprising an amino acid sequence of SEQ ID NO: 261 and a third polypeptide comprising an amino acid sequence of SEQ ID NO: 124, and the second antigen binding molecule comprises a first polypeptide comprising an amino acid sequence of SEQ ID NO: 106, a second polypeptide comprising an amino acid sequence of SEQ ID NO: 112 and a third polypeptide comprising an amino acid sequence of SEQ ID NO: 254; or
h) the first antigen binding molecule comprises a first polypeptide comprising an amino acid sequence of SEQ ID NO: 106, a second polypeptide comprising an amino acid sequence of SEQ ID NO: 119 and a third polypeptide comprising an amino acid sequence of SEQ ID NO: 257, and the second antigen binding molecule comprises a first polypeptide comprising an amino acid sequence of SEQ ID NO: 106, a second polypeptide comprising an amino acid sequence of SEQ ID NO: 258 and a third polypeptide comprising an amino acid sequence of SEQ ID NO: 127.

29. The pair of antigen binding molecules according to claim 1, wherein the first and the second target-binding domains specifically bind to PD-1, and the first and second cytokine receptor subunits are subunits of the IL-2 receptor complex.

30. The pair of antigen binding molecules according to claim 1, wherein
a) the first antigen binding molecule comprises a first polypeptide comprising an amino acid sequence of SEQ ID NO: 86, a second polypeptide comprising an amino acid sequence of SEQ ID NO: 44 and a third polypeptide comprising an amino acid sequence of SEQ ID NO: 83, and the second antigen binding molecule comprises a first polypeptide comprising an amino acid sequence of SEQ ID NO: 28, a second polypeptide comprising an amino acid sequence of SEQ ID NO: 88 and a third polypeptide comprising an amino acid sequence of SEQ ID NO: 87; or
b) the first antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89, and the second antigen binding molecule comprises a first polypeptide comprising an amino acid sequence of SEQ ID NO: 92, a second polypeptide comprising an amino acid sequence of SEQ ID NO: 44 and a third polypeptide comprising an amino acid sequence of SEQ ID NO: 91; or
c) the first antigen binding molecule comprises a first polypeptide comprising an amino acid sequence of SEQ ID NO: 28, a second polypeptide comprising an amino acid sequence of SEQ ID NO: 96 and a third polypeptide comprising an amino acid sequence of SEQ ID NO: 95, and the second antigen binding molecule comprises a first polypeptide comprising an amino acid sequence of SEQ ID NO: 28, a second polypeptide comprising an amino acid sequence of SEQ ID NO: 101 and a third polypeptide comprising an amino acid sequence of SEQ ID NO: 89; or
d) the first antigen binding molecule comprises a first polypeptide comprising an amino acid sequence of SEQ ID NO: 28, a second polypeptide comprising an amino acid sequence of SEQ ID NO: 99 and a third polypeptide comprising an amino acid sequence of SEQ ID NO: 98, and the second antigen binding molecule comprises a first polypeptide comprising an amino acid sequence of SEQ ID NO: 28, a second polypeptide comprising an amino acid sequence of SEQ ID NO: 88 and a third polypeptide comprising an amino acid sequence of SEQ ID NO: 102; or
e) the first antigen binding molecule comprises a first polypeptide comprising an amino acid sequence of SEQ ID NO: 28, a second polypeptide comprising an amino acid sequence of SEQ ID NO: 96 and a third polypeptide comprising an amino acid sequence of SEQ ID NO: 97, and the second antigen binding molecule comprises a first polypeptide comprising an amino acid sequence of SEQ ID NO: 28, a second polypeptide comprising an amino acid sequence of SEQ ID NO: 103 and a third polypeptide comprising an amino acid sequence of SEQ ID NO: 89; or
f) the first antigen binding molecule comprises a first polypeptide comprising an amino acid sequence of SEQ ID NO: 28, a second polypeptide comprising an amino acid sequence of SEQ ID NO: 100 and a third polypeptide comprising an amino acid sequence of SEQ ID NO: 98, and the second antigen binding molecule comprises a first polypeptide comprising an amino acid sequence of SEQ ID NO: 28, a second polypeptide comprising an amino acid sequence of SEQ ID NO: 88 and a third polypeptide comprising an amino acid sequence of SEQ ID NO: 104; or
g) the first antigen binding molecule comprises a first polypeptide comprising an amino acid sequence of SEQ ID NO: 28, a second polypeptide comprising an amino acid sequence of SEQ ID NO: 98 and a third polypeptide comprising an amino acid sequence of SEQ ID NO: 99, and the second antigen binding molecule comprises a first polypeptide comprising an amino acid sequence of SEQ ID NO: 28, a second polypeptide comprising an amino acid sequence of SEQ ID NO: 101 and a third polypeptide comprising an amino acid sequence of SEQ ID NO: 89; or
h) the first antigen binding molecule comprises a first polypeptide comprising an amino acid sequence of SEQ ID NO: 28, a second polypeptide comprising an amino acid sequence of SEQ ID NO: 96 and a third polypeptide comprising an amino acid sequence of SEQ ID NO: 95, and the second antigen binding molecule comprises a first polypeptide comprising an amino acid sequence of SEQ ID NO: 28, a second polypeptide comprising an amino acid sequence of SEQ ID NO: 88 and a third polypeptide comprising an amino acid sequence of SEQ ID NO: 102; or
i) the first antigen binding molecule comprises a first polypeptide comprising an amino acid sequence of SEQ ID NO: 28, a second polypeptide comprising an amino acid sequence of SEQ ID NO: 100 and a third polypeptide comprising an amino acid sequence of SEQ ID NO: 98, and the second antigen binding molecule comprises a first polypeptide comprising an amino acid sequence of SEQ ID NO: 28, a second polypeptide comprising an amino acid sequence of SEQ ID NO: 103 and a third polypeptide comprising an amino acid sequence of SEQ ID NO: 89; or
j) the first antigen binding molecule comprises a first polypeptide comprising an amino acid sequence of SEQ ID NO: 28, a second polypeptide comprising an amino acid sequence of SEQ ID NO: 96 and a third polypeptide comprising an amino acid sequence of SEQ ID NO: 97, and the second antigen binding molecule comprises a first polypeptide comprising an amino acid sequence of SEQ ID NO: 28, a second polypeptide comprising an amino acid sequence of SEQ ID NO: 88 and a third polypeptide comprising an amino acid sequence of SEQ ID NO: 104.

31. The pair of antigen binding molecules according to claim 1, wherein the first and the second target-binding domains specifically bind to LAG-3, and the first and second cytokine receptor subunits are subunits of the IL-2 receptor complex.

32. The pair of antigen binding molecules according to claim 1, wherein
a) first antigen binding molecule comprises a first polypeptide comprising an amino acid sequence of SEQ ID NO: 106, a second polypeptide comprising an amino acid sequence of SEQ ID NO: 155 and a third polypeptide comprising an amino acid sequence of SEQ ID NO: 154, and the second antigen binding molecule comprises a first polypeptide comprising an amino acid sequence of SEQ ID NO: 106, a second polypeptide comprising an amino acid sequence of SEQ ID NO: 165 and a third polypeptide comprising an amino acid sequence of SEQ ID NO: 163; or
b) the first antigen binding molecule comprises a first polypeptide comprising an amino acid sequence of SEQ ID NO: 106, a second polypeptide comprising an amino acid sequence of SEQ ID NO: 161 and a third polypeptide comprising an amino acid sequence of SEQ ID NO: 160, and the second antigen binding molecule comprises a first polypeptide comprising an amino acid sequence of SEQ ID NO: 106, a second polypeptide comprising an amino acid sequence of SEQ ID NO: 158 and a third polypeptide comprising an amino acid sequence of SEQ ID NO: 159; or
c) the first antigen binding molecule comprises a first polypeptide comprising an amino acid sequence of SEQ ID NO: 106, a second polypeptide comprising an amino acid sequence of SEQ ID NO: 155 and a third polypeptide comprising an amino acid sequence of SEQ ID NO: 156, and the second antigen binding molecule comprises a first polypeptide comprising an amino acid sequence of SEQ ID NO: 106, a second polypeptide comprising an amino acid sequence of SEQ ID NO: 164 and a third polypeptide comprising an amino acid sequence of SEQ ID NO: 163; or
d) the first antigen binding molecule comprises a first polypeptide comprising an amino acid sequence of SEQ ID NO: 106, a second polypeptide comprising an amino acid sequence of SEQ ID NO: 162 and a third polypeptide comprising an amino acid sequence of SEQ ID NO: 160, and the second antigen binding molecule comprises a first polypeptide comprising an amino acid sequence of SEQ ID NO: 106, a second polypeptide comprising an amino acid sequence of SEQ ID NO: 158 and a third polypeptide comprising an amino acid sequence of SEQ ID NO: 157; or
e) the first antigen binding molecule comprises a first polypeptide comprising an amino acid sequence of SEQ ID NO: 106, a second polypeptide comprising an amino acid sequence of SEQ ID NO: 161 and a third polypeptide comprising an amino acid sequence of SEQ ID NO: 160, and the second antigen binding molecule comprises a first polypeptide comprising an amino acid sequence of SEQ ID NO: 106, a second polypeptide comprising an amino acid sequence of SEQ ID NO: 165 and a third polypeptide comprising an amino acid sequence of SEQ ID NO: 163; or
f) the first antigen binding molecule comprises a first polypeptide comprising an amino acid sequence of SEQ ID NO: 106, a second polypeptide comprising an amino acid sequence of SEQ ID NO: 155 and a third polypeptide comprising an amino acid sequence of SEQ ID NO: 154, and the second antigen binding molecule comprises a first polypeptide comprising an amino acid sequence of SEQ ID NO: 106, a second polypeptide comprising an amino acid sequence of SEQ ID NO: 158 and a third polypeptide comprising an amino acid sequence of SEQ ID NO: 159; or
g) the first antigen binding molecule comprises a first polypeptide comprising an amino acid sequence of SEQ ID NO: 106, a second polypeptide comprising an amino acid sequence of SEQ ID NO: 162 and a third polypeptide comprising an amino acid sequence of SEQ ID NO: 160, and the second antigen binding molecule comprises a first polypeptide comprising an amino acid sequence of SEQ ID NO: 106, a second polypeptide comprising an amino acid sequence of SEQ ID NO: 164 and a third polypeptide comprising an amino acid sequence of SEQ ID NO: 163; or
h) the first antigen binding molecule comprises a first polypeptide comprising an amino acid sequence of SEQ ID NO: 106, a second polypeptide comprising an amino acid sequence of SEQ ID NO: 155 and a third polypeptide comprising an amino acid sequence of SEQ ID NO: 156, and the second antigen binding molecule comprises a first polypeptide comprising an amino acid sequence of SEQ ID NO: 106, a second polypeptide comprising an amino acid sequence of SEQ ID NO: 158 and a third polypeptide comprising an amino acid sequence of SEQ ID NO: 157.

33. The pair of antigen binding molecules according to claim 1, wherein the first and the second target-binding domains specifically bind to EGFR, and the first and second cytokine receptor subunits are subunits of the IL-2 receptor complex.

34. The pair of antigen binding molecules according to claim 1, wherein
a) first antigen binding molecule comprises a first polypeptide comprising an amino acid sequence of SEQ ID NO: 106, a second polypeptide comprising an amino acid sequence of SEQ ID NO: 155 and a third polypeptide comprising an amino acid sequence of SEQ ID NO: 154, and the second antigen binding molecule comprises a first polypeptide comprising an amino acid sequence of SEQ ID NO: 106, a second polypeptide comprising an amino acid sequence of SEQ ID NO: 165 and a third polypeptide comprising an amino acid sequence of SEQ ID NO: 163; or
b) the first antigen binding molecule comprises a first polypeptide comprising an amino acid sequence of SEQ ID NO: 106, a second polypeptide comprising an amino acid sequence of SEQ ID NO: 161 and a third polypeptide comprising an amino acid sequence of SEQ ID NO: 160, and the second antigen binding molecule comprises a first polypeptide comprising an amino acid sequence of SEQ ID NO: 106, a second polypeptide comprising an amino acid sequence of SEQ ID NO: 158 and a third polypeptide comprising an amino acid sequence of SEQ ID NO: 159; or
c) the first antigen binding molecule comprises a first polypeptide comprising an amino acid sequence of SEQ ID NO: 106, a second polypeptide comprising an amino acid sequence of SEQ ID NO: 155 and a third polypeptide comprising an amino acid sequence of SEQ ID NO: 156, and the second antigen binding molecule comprises a first polypeptide comprising an amino acid sequence of SEQ ID NO: 106, a second polypeptide comprising an amino acid sequence of SEQ ID NO: 164 and a third polypeptide comprising an amino acid sequence of SEQ ID NO: 163; or
d) the first antigen binding molecule comprises a first polypeptide comprising an amino acid sequence of SEQ ID NO: 106, a second polypeptide comprising an amino acid sequence of SEQ ID NO: 162 and a third polypeptide comprising an amino acid sequence of SEQ ID NO: 160, and the second antigen binding molecule comprises a first polypeptide comprising an amino acid sequence of SEQ ID NO: 106, a second polypeptide comprising an amino acid sequence of SEQ ID NO: 158 and a third polypeptide comprising an amino acid sequence of SEQ ID NO: 157; or
e) the first antigen binding molecule comprises a first polypeptide comprising an amino acid sequence of SEQ ID NO: 106, a second polypeptide comprising an amino acid sequence of SEQ ID NO: 161 and a third polypeptide comprising an amino acid sequence of SEQ ID NO: 160, and the second antigen binding molecule comprises a first polypeptide comprising an amino acid sequence of SEQ ID NO: 106, a second polypeptide comprising an amino acid sequence of SEQ ID NO: 165 and a third polypeptide comprising an amino acid sequence of SEQ ID NO: 163; or
f) the first antigen binding molecule comprises a first polypeptide comprising an amino acid sequence of SEQ ID NO: 106, a second polypeptide comprising an amino acid sequence of SEQ ID NO: 155 and a third polypeptide comprising an amino acid sequence of SEQ ID NO: 154, and the second antigen binding molecule comprises a first polypeptide comprising an amino acid sequence of SEQ ID NO: 106, a second polypeptide comprising an amino acid sequence of SEQ ID NO: 158 and a third polypeptide comprising an amino acid sequence of SEQ ID NO: 159; or
g) the first antigen binding molecule comprises a first polypeptide comprising an amino acid sequence of SEQ ID NO: 106, a second polypeptide comprising an amino acid sequence of SEQ ID NO: 162 and a third polypeptide comprising an amino acid sequence of SEQ ID NO: 160, and the second antigen binding molecule comprises a first polypeptide comprising an amino acid sequence of SEQ ID NO: 106, a second polypeptide comprising an amino acid sequence of SEQ ID NO: 164 and a third polypeptide comprising an amino acid sequence of SEQ ID NO: 163; or
h) the first antigen binding molecule comprises a first polypeptide comprising an amino acid sequence of SEQ ID NO: 106, a second polypeptide comprising an amino acid sequence of SEQ ID NO: 155 and a third polypeptide comprising an amino acid sequence of SEQ ID NO: 156, and the second antigen binding molecule comprises a first polypeptide comprising an amino acid sequence of SEQ ID NO: 106, a second polypeptide comprising an amino acid sequence of SEQ ID NO: 158 and a third polypeptide comprising an amino acid sequence of SEQ ID NO: 157.

35. The pair of antigen binding molecules according to claim 1, wherein the first and the second target-binding domains specifically bind to FAP, and the first and second cytokine receptor subunits are subunits of the IL-2 receptor complex.

36. The pair of antigen binding molecules according to claim 1, wherein
a) the first antigen binding molecule comprises a first polypeptide comprising an amino acid sequence of SEQ ID NO: 106, a second polypeptide comprising an amino acid sequence of SEQ ID NO: 110 and a third polypeptide comprising an amino acid sequence of SEQ ID NO: 25, and the second antigen binding molecule comprises a first polypeptide comprising an amino acid sequence of SEQ ID NO: 106, a second polypeptide comprising an amino acid sequence of SEQ ID NO: 113 and a third polypeptide comprising an amino acid sequence of SEQ ID NO: 29; or
b) the first antigen binding molecule comprises a first polypeptide comprising an amino acid sequence of SEQ ID NO: 106, a second polypeptide comprising an amino acid sequence of SEQ ID NO: 32 and a third polypeptide comprising an amino acid sequence of SEQ ID NO: 105, and the second antigen binding molecule comprises a first polypeptide comprising an amino acid sequence of SEQ ID NO: 106, a second polypeptide comprising an amino acid sequence of SEQ ID NO: 34 and a third polypeptide comprising an amino acid sequence of SEQ ID NO: 109; or
c) the first antigen binding molecule comprises a first polypeptide comprising an amino acid sequence of SEQ ID NO: 106, a second polypeptide comprising an amino acid sequence of SEQ ID NO: 111 and a third polypeptide comprising an amino acid sequence of SEQ ID NO: 25, and the second antigen binding molecule comprises a first polypeptide comprising an amino acid sequence of SEQ ID NO: 106, a second polypeptide comprising an amino acid sequence of SEQ ID NO: 112 and a third polypeptide comprising an amino acid sequence of SEQ ID NO: 29; or
d) the first antigen binding molecule comprises a first polypeptide comprising an amino acid sequence of SEQ ID NO: 106, a second polypeptide comprising an amino acid sequence of SEQ ID NO: 32 and a third polypeptide comprising an amino acid sequence of SEQ ID NO: 107, and the second antigen binding molecule comprises a first polypeptide comprising an amino acid sequence of SEQ ID NO: 106, a second polypeptide comprising an amino acid sequence of SEQ ID NO: 34 and a third polypeptide comprising an amino acid sequence of SEQ ID NO: 108; or
e) the first antigen binding molecule comprises a first polypeptide comprising an amino acid sequence of SEQ ID NO: 106, a second polypeptide comprising an amino acid sequence of SEQ ID NO: 110 and a third polypeptide comprising an amino acid sequence of SEQ ID NO: 25, and the second antigen binding molecule comprises a first polypeptide comprising an amino acid sequence of SEQ ID NO: 106, a second polypeptide comprising an amino acid sequence of SEQ ID NO: 34 and a third polypeptide comprising an amino acid sequence of SEQ ID NO: 109; or
f) the first antigen binding molecule comprises a first polypeptide comprising an amino acid sequence of SEQ ID NO: 106, a second polypeptide comprising an amino acid sequence of SEQ ID NO: 32 and a third polypeptide comprising an amino acid sequence of SEQ ID NO: 105, and the second antigen binding molecule comprises a first polypeptide comprising an amino acid sequence of SEQ ID NO: 106, a second polypeptide comprising an amino acid sequence of SEQ ID NO: 113 and a third polypeptide comprising an amino acid sequence of SEQ ID NO: 29; or
g) the first antigen binding molecule comprises a first polypeptide comprising an amino acid sequence of SEQ ID NO: 106, a second polypeptide comprising an amino acid sequence of SEQ ID NO: 111 and a third polypeptide comprising an amino acid sequence of SEQ ID NO: 25, and the second antigen binding molecule comprises a first polypeptide comprising an amino acid sequence of SEQ ID NO: 106, a second polypeptide comprising an amino acid sequence of SEQ ID NO: 34 and a third polypeptide comprising an amino acid sequence of SEQ ID NO: 108; or
h) the first antigen binding molecule comprises a first polypeptide comprising an amino acid sequence of SEQ ID NO: 106, a second polypeptide comprising an amino acid sequence of SEQ ID NO: 32 and a third polypeptide comprising an amino acid sequence of SEQ ID NO: 107, and the second antigen binding molecule comprises a first polypeptide comprising an amino acid sequence of SEQ ID NO: 106, a second polypeptide comprising an amino acid sequence of SEQ ID NO: 112 and a third polypeptide comprising an amino acid sequence of SEQ ID NO: 29.

37. The pair of antigen binding molecules according to claim 1, wherein the first and the second target-binding domains specifically bind to Her2, and the first and second cytokine receptor subunits are subunits of the IL-2 receptor complex.

38. The pair of antigen binding molecules according to claim 1, wherein
a) first antigen binding molecule comprises a first polypeptide comprising an amino acid sequence of SEQ ID NO: 106, a second polypeptide comprising an amino acid sequence of SEQ ID NO: 136 and a third polypeptide comprising an amino acid sequence of SEQ ID NO: 134, and the second antigen binding molecule comprises a first polypeptide comprising an amino acid sequence of SEQ ID NO: 106, a second polypeptide comprising an amino acid sequence of SEQ ID NO: 138 and a third polypeptide comprising an amino acid sequence of SEQ ID NO: 137; or
b) the first antigen binding molecule comprises a first polypeptide comprising an amino acid sequence of SEQ ID NO: 106, a second polypeptide comprising an amino acid sequence of SEQ ID NO: 135 and a third polypeptide comprising an amino acid sequence of SEQ ID NO: 134, and the second antigen binding molecule comprises a first polypeptide comprising an amino acid sequence of SEQ ID NO: 106, a second polypeptide comprising an amino acid sequence of SEQ ID NO: 138 and a third polypeptide comprising an amino acid sequence of SEQ ID NO: 139.

39. The pair of antigen binding molecules according to claim 1, wherein the first and the second target-binding domains specifically bind to Her3, and the first and second cytokine receptor subunits are subunits of the IL-2 receptor complex.

40. The pair of antigen binding molecules according to claim 1, wherein
a) the first antigen binding molecule comprises a first polypeptide comprising an amino acid sequence of SEQ ID NO: 106, a second polypeptide comprising an amino acid sequence of SEQ ID NO: 146 and a third polypeptide comprising an amino acid sequence of SEQ ID NO: 144, and the second antigen binding molecule comprises a first polypeptide comprising an amino acid sequence of SEQ ID NO: 106, a second polypeptide comprising an amino acid sequence of SEQ ID NO: 148 and a third polypeptide comprising an amino acid sequence of SEQ ID NO: 147; or
b) the first antigen binding molecule comprises a first polypeptide comprising an amino acid sequence of SEQ ID NO: 106, a second polypeptide comprising an amino acid sequence of SEQ ID NO: 145 and a third polypeptide comprising an amino acid sequence of SEQ ID NO: 144, and the second antigen binding molecule comprises a first polypeptide comprising an amino acid sequence of SEQ ID NO: 106, a second polypeptide comprising an amino acid sequence of SEQ ID NO: 148 and a third polypeptide comprising an amino acid sequence of SEQ ID NO: 149.

41. The pair of antigen binding molecules according to claim 1, wherein the first and the second target-binding domains specifically bind to PD-1, and the first and second cytokine receptor subunits are subunits of the IL-7 receptor complex.

42. The pair of antigen binding molecules according to claim 1, wherein
a) the first antigen binding molecule comprises a first polypeptide comprising an amino acid sequence of SEQ ID NO: 106, a second polypeptide comprising an amino acid sequence of SEQ ID NO: 194 and a third polypeptide comprising an amino acid sequence of SEQ ID NO: 193, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89; or
b) the first antigen binding molecule comprises a first polypeptide comprising an amino acid sequence of SEQ ID NO: 106, a second polypeptide comprising an amino acid sequence of SEQ ID NO: 195 and a third polypeptide comprising an amino acid sequence of SEQ ID NO: 124, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89; or
c) the first antigen binding molecule comprises a first polypeptide comprising an amino acid sequence of SEQ ID NO: 106, a second polypeptide comprising an amino acid sequence of SEQ ID NO: 196 and a third polypeptide comprising an amino acid sequence of SEQ ID NO: 124, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89; or
d) the first antigen binding molecule comprises a first polypeptide comprising an amino acid sequence of SEQ ID NO: 106, a second polypeptide comprising an amino acid sequence of SEQ ID NO: 197 and a third polypeptide comprising an amino acid sequence of SEQ ID NO: 124, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89; or
d) the first antigen binding molecule comprises a first polypeptide comprising an amino acid sequence of SEQ ID NO: 106, a second polypeptide comprising an amino acid sequence of SEQ ID NO: 198 and a third polypeptide comprising an amino acid sequence of SEQ ID NO: 124, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89; or
e) the first antigen binding molecule comprises a first polypeptide comprising an amino acid sequence of SEQ ID NO: 106, a second polypeptide comprising an amino acid sequence of SEQ ID NO: 199 and a third polypeptide comprising an amino acid sequence of SEQ ID NO: 124, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89; or
f) the first antigen binding molecule comprises a first polypeptide comprising an amino acid sequence of SEQ ID NO: 106, a second polypeptide comprising an amino acid sequence of SEQ ID NO: 200 and a third polypeptide comprising an amino acid sequence of SEQ ID NO: 124, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89; or
g) the first antigen binding molecule comprises a first polypeptide comprising an amino acid sequence of SEQ ID NO: 106, a second polypeptide comprising an amino acid sequence of SEQ ID NO: 201 and a third polypeptide comprising an amino acid sequence of SEQ ID NO: 124, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89; or
h) the first antigen binding molecule comprises a first polypeptide comprising an amino acid sequence of SEQ ID NO: 106, a second polypeptide comprising an amino acid sequence of SEQ ID NO: 202 and a third polypeptide comprising an amino acid sequence of SEQ ID NO: 124, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89; or
i) the first antigen binding molecule comprises a first polypeptide comprising an amino acid sequence of SEQ ID NO: 106, a second polypeptide comprising an amino acid sequence of SEQ ID NO: 203 and a third polypeptide comprising an amino acid sequence of SEQ ID NO: 124, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89; or
j) the first antigen binding molecule comprises a first polypeptide comprising an amino acid sequence of SEQ ID NO: 106, a second polypeptide comprising an amino acid sequence of SEQ ID NO: 204 and a third polypeptide comprising an amino acid sequence of SEQ ID NO: 124, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89; or
k) the first antigen binding molecule comprises a first polypeptide comprising an amino acid sequence of SEQ ID NO: 106, a second polypeptide comprising an amino acid sequence of SEQ ID NO: 205 and a third polypeptide comprising an amino acid sequence of SEQ ID NO: 124, and the second antigen binding molecule comprises a first polypeptide comprising an amino acid sequence of SEQ ID NO: 28, a second polypeptide comprising an amino acid sequence of SEQ ID NO: 90 and a third polypeptide comprising an amino acid sequence of SEQ ID NO: 89.

43. The pair of antigen binding molecules according to claim 1, wherein the first target-binding domain is fused at its C-terminus of CH1₁ to the N-terminus of the first Fc domain subunit and the first cytokine receptor-binding domain is fused at its C-terminus to the N-terminus of the second Fc domain subunit and the second target-binding domain is fused at its C-terminus of CH1₂ to the N-terminus of the first Fc domain subunit and the second cytokine receptor-binding domain is fused at its C-terminus to the N-terminus of the second Fc domain subunit.

44. The pair of antigen binding molecules according to claim 1, wherein
a) the first antigen binding molecule comprises a first polypeptide comprising in order from the N-terminus to C-terminus VH₁, CH1₁ and a first Fc domain subunit, a second polypeptide comprising in order from the N-terminus to C-terminus VL₁ and CL₁, and a third polypeptide comprising in order from the N-terminus to C-terminus the first cytokine receptor-binding domain and a second Fc domain subunit, and the second antigen binding molecule comprises a first polypeptide comprising in order from the N-terminus to C-terminus VH₂, CH1₂ and a first Fc domain subunit, a second polypeptide comprising in order from the N-terminus to C-terminus VL₂ and CL₂, and a third polypeptide comprising in order from the N-terminus to C-terminus the second cytokine receptor-binding domain and a second Fc domain subunit; or
b) the first antigen binding molecule comprises a first polypeptide comprising in order from the N-terminus to C-terminus VL₁, CH1₁ and a first Fc domain subunit, a second polypeptide comprising in order from the N-terminus to C-terminus VH₁ and CL₁, and a third polypeptide comprising in order from the N-terminus to C-terminus the first cytokine receptor-binding domain and a second Fc domain subunit, and the second antigen binding molecule comprises a first polypeptide comprising in order from the N-terminus to C-terminus VH₂, CH1₂ and a first Fc domain subunit, a second polypeptide comprising in order from the N-terminus to C-terminus VL₂ and CL₂, and a third polypeptide comprising in order from the N-terminus to C-terminus the second cytokine receptor-binding domain and a second Fc domain subunit;
wherein VH₁, VL₁, CH1₁ and CL₁ form the first target-binding domain and VH₂, VL₂, CH1₂, and CL₂, form the second target-binding domain.

45. The pair of antigen binding molecules according to claim 1, wherein
a) the first antigen binding molecule comprises a first polypeptide comprising an amino acid sequence of SEQ ID NO: 32, a second polypeptide comprising an amino acid sequence of SEQ ID NO: 25 and a third polypeptide comprising an amino acid sequence of SEQ ID NO: 39, and the second antigen binding molecule comprises a first polypeptide comprising an amino acid sequence of SEQ ID NO: 34, a second polypeptide comprising an amino acid sequence of SEQ ID NO: 29 and a third polypeptide comprising an amino acid sequence of SEQ ID NO: 40; or
b) the first antigen binding molecule comprises a first polypeptide comprising an amino acid sequence of SEQ ID NO: 34, a second polypeptide comprising an amino acid sequence of SEQ ID NO: 29 and a third polypeptide comprising an amino acid sequence of SEQ ID NO: 39, and the second antigen binding molecule comprises a first polypeptide comprising an amino acid sequence of SEQ ID NO: 32, a second polypeptide comprising an amino acid sequence of SEQ ID NO: 25 and a third polypeptide comprising an amino acid sequence of SEQ ID NO: 40; or
c) the first antigen binding molecule comprises a first polypeptide comprising an amino acid sequence of SEQ ID NO: 237, a second polypeptide comprising an amino acid sequence of SEQ ID NO: 32 and a third polypeptide comprising an amino acid sequence of SEQ ID NO: 25, and the second antigen binding molecule comprises a first polypeptide comprising an amino acid sequence of SEQ ID NO: 238, a second polypeptide comprising an amino acid sequence of SEQ ID NO: 34 and a third polypeptide comprising an amino acid sequence of SEQ ID NO: 29; or
d) the first antigen binding molecule comprises a first polypeptide comprising an amino acid sequence of SEQ ID NO: 235, a second polypeptide comprising an amino acid sequence of SEQ ID NO: 32 and a third polypeptide comprising an amino acid sequence of SEQ ID NO: 25, and the second antigen binding molecule comprises a first polypeptide comprising an amino acid sequence of SEQ ID NO: 236, a second polypeptide comprising an amino acid sequence of SEQ ID NO: 34 and a third polypeptide comprising an amino acid sequence of SEQ ID NO: 29; or
e) the first antigen binding molecule comprises a first polypeptide comprising an amino acid sequence of SEQ ID NO: 239, a second polypeptide comprising an amino acid sequence of SEQ ID NO: 32 and a third polypeptide comprising an amino acid sequence of SEQ ID NO: 25, and the second antigen binding molecule comprises a first polypeptide comprising an amino acid sequence of SEQ ID NO: 240, a second polypeptide comprising an amino acid sequence of SEQ ID NO: 34 and a third polypeptide comprising an amino acid sequence of SEQ ID NO: 29; or
f) the first antigen binding molecule comprises a first polypeptide comprising an amino acid sequence of SEQ ID NO: 235, a second polypeptide comprising an amino acid sequence of SEQ ID NO: 32 and a third polypeptide comprising an amino acid sequence of SEQ ID NO: 25, and the second antigen binding molecule comprises a first polypeptide comprising an amino acid sequence of SEQ ID NO: 237, a second polypeptide comprising an amino acid sequence of SEQ ID NO: 34 and a third polypeptide comprising an amino acid sequence of SEQ ID NO: 29; or
g) the first antigen binding molecule comprises a first polypeptide comprising an amino acid sequence of SEQ ID NO: 236, a second polypeptide comprising an amino acid sequence of SEQ ID NO: 32 and a third polypeptide comprising an amino acid sequence of SEQ ID NO: 25, and the second antigen binding molecule comprises a first polypeptide comprising an amino acid sequence of SEQ ID NO: 235, a second polypeptide comprising an amino acid sequence of SEQ ID NO: 34 and a third polypeptide comprising an amino acid sequence of SEQ ID NO: 29; or
h) the first antigen binding molecule comprises a first polypeptide comprising an amino acid sequence of SEQ ID NO: 240, a second polypeptide comprising an amino acid sequence of SEQ ID NO: 32 and a third polypeptide comprising an amino acid sequence of SEQ ID NO: 25, and the second antigen binding molecule comprises a first polypeptide comprising an amino acid sequence of SEQ ID NO: 239, a second polypeptide comprising an amino acid sequence of SEQ ID NO: 34 and a third polypeptide comprising an amino acid sequence of SEQ ID NO: 29.

46. The pair of antigen binding molecules according to claim 1, wherein the first target-binding domain is fused at its C-terminus of CH1₁ to the N-terminus of one of the Fc domain subunits and the first cytokine receptor-binding domain is fused at its N-terminus to the C-terminus of the same Fc domain subunit and the second target-binding domain is fused at its C-terminus of CH1₂ to the N-terminus of one of the Fc domain subunits and the second cytokine receptor-binding domain is fused at its N-terminus to the C-terminus of the same Fc domain subunit.

47. The pair of antigen binding molecules according to claim 1, wherein
a) the first antigen binding molecule comprises a first polypeptide comprising a first Fc domain subunit, a second polypeptide comprising in order from the N-terminus to C-terminus a VH₁, a CH1₁, a second Fc domain subunit and a the first cytokine receptor-binding domain, and a third polypeptide comprising in order from the N-terminus to C-terminus a VL₁ and a CL₁; and the second antigen binding molecule comprises a first polypeptide comprising a first Fc domain subunit, a second polypeptide comprising in order from the N-terminus to C-terminus a VH₂, a CH1₂, a second Fc domain subunit and a the second cytokine receptor-binding domain, and a third polypeptide comprising in order from the N-terminus to C-terminus VL₂ and CL₂; or
b) the first antigen binding molecule comprises a first polypeptide comprising a first Fc domain subunit, a second polypeptide comprising in order from the N-terminus to C-terminus a VL₁, a CH1₁, a second Fc domain subunit and a the first cytokine receptor-binding domain, and a third polypeptide comprising in order from the N-terminus to C-terminus a VH₁ and a CL₁; and the second antigen binding molecule comprises a first polypeptide comprising a first Fc domain subunit, a second polypeptide comprising in order from the N-terminus to C-terminus a VH₂, a CH1₂, a second Fc domain subunit and the second cytokine receptor-binding domain, and a third polypeptide comprising in order from the N-terminus to C-terminus a VL₂ and a CL₂; or
c) the first antigen binding molecule comprises a first polypeptide comprising a first Fc domain subunit, a second polypeptide comprising in order from the N-terminus to C-terminus a VL₁, a CH1₁, a second Fc domain subunit and a the first cytokine receptor-binding domain, and a third polypeptide comprising in order from the N-terminus to C-terminus a VH₁ and a CL₁; and the second antigen binding molecule comprises a first polypeptide comprising a first Fc domain subunit, a second polypeptide comprising in order from the N-terminus to C-terminus a VH₂, a CH1₂, a second Fc domain subunit and a the second cytokine receptor-binding domain, and a third polypeptide comprising in order from the N-terminus to C-terminus VL₂ and CL₂;
wherein VH₁, VL₁, CH1₁ and CL₁ form the first target-binding domain and VH₂, VL₂, CH1₂, and CL₂, form the second target-binding domain.

48. The pair of antigen binding molecules according to claim 1, wherein
a) the first antigen binding molecule comprises a first polypeptide comprising an amino acid sequence of SEQ ID NO: 28, a second polypeptide comprising an amino acid sequence of SEQ ID NO: 42 and a third polypeptide comprising an amino acid sequence of SEQ ID NO: 41, and the second antigen binding molecule comprises a first polypeptide comprising an amino acid sequence of SEQ ID NO: 45, a second polypeptide comprising an amino acid sequence of SEQ ID NO: 44 and a third polypeptide comprising an amino acid sequence of SEQ ID NO: 43; or
b) the first antigen binding molecule comprises a first polypeptide comprising an amino acid sequence of SEQ ID NO: 28, a second polypeptide comprising an amino acid sequence of SEQ ID NO: 46 and a third polypeptide comprising an amino acid sequence of SEQ ID NO: 41, and the second antigen binding molecule comprises a first polypeptide comprising an amino acid sequence of SEQ ID NO: 47, a second polypeptide comprising an amino acid sequence of SEQ ID NO: 44 and a third polypeptide comprising an amino acid sequence of SEQ ID NO: 43; or
c) the first antigen binding molecule comprises a first polypeptide comprising an amino acid sequence of SEQ ID NO: 241, a second polypeptide comprising an amino acid sequence of SEQ ID NO: 246 and a third polypeptide comprising an amino acid sequence of SEQ ID NO: 25, and the second antigen binding molecule comprises a first polypeptide comprising an amino acid sequence of SEQ ID NO: 241, a second polypeptide comprising an amino acid sequence of SEQ ID NO: 249 and a third polypeptide comprising an amino acid sequence of SEQ ID NO: 29; or
d) the first antigen binding molecule comprises a first polypeptide comprising an amino acid sequence of SEQ ID NO: 241, a second polypeptide comprising an amino acid sequence of SEQ ID NO: 242 and a third polypeptide comprising an amino acid sequence of SEQ ID NO: 25, and the second antigen binding molecule comprises a first polypeptide comprising an amino acid sequence of SEQ ID NO: 241, a second polypeptide comprising an amino acid sequence of SEQ ID NO: 245 and a third polypeptide comprising an amino acid sequence of SEQ ID NO: 29; or
e) the first antigen binding molecule comprising a first polypeptide comprises an amino acid sequence of SEQ ID NO: 241, a second polypeptide comprising an amino acid sequence of SEQ ID NO: 250 and a third polypeptide comprising an amino acid sequence of SEQ ID NO: 25, and the second antigen binding molecule comprises a first polypeptide comprising an amino acid sequence of SEQ ID NO: 241, a second polypeptide comprising an amino acid sequence of SEQ ID NO: 253 and a third polypeptide comprising an amino acid sequence of SEQ ID NO: 29; or
f) the first antigen binding molecule comprises a first polypeptide comprising an amino acid sequence of SEQ ID NO: 241, a second polypeptide comprising an amino acid sequence of SEQ ID NO: 248 and a third polypeptide comprising an amino acid sequence of SEQ ID NO: 25, and the second antigen binding molecule comprises a first polypeptide comprising an amino acid sequence of SEQ ID NO: 241, a second polypeptide comprising an amino acid sequence of SEQ ID NO: 247 and a third polypeptide comprising an amino acid sequence of SEQ ID NO: 29; or
g) the first antigen binding molecule comprises a first polypeptide comprising an amino acid sequence of SEQ ID NO: 241, a second polypeptide comprising an amino acid sequence of SEQ ID NO: 244 and a third polypeptide comprising an amino acid sequence of SEQ ID NO: 25, and the second antigen binding molecule comprises a first polypeptide comprising an amino acid sequence of SEQ ID NO: 241, a second polypeptide comprising an amino acid sequence of SEQ ID NO: 243 and a third polypeptide comprising an amino acid sequence of SEQ ID NO: 29; or
h) the first antigen binding molecule comprises a first polypeptide comprising an amino acid sequence of SEQ ID NO: 241, a second polypeptide comprising an amino acid sequence of SEQ ID NO: 252 and a third polypeptide comprising an amino acid sequence of SEQ ID NO: 25, and the second antigen binding molecule comprises a first polypeptide comprising an amino acid sequence of SEQ ID NO: 241, a second polypeptide comprising an amino acid sequence of SEQ ID NO: 251 and a third polypeptide comprising an amino acid sequence of SEQ ID NO: 29; or
i) the first antigen binding molecule comprises a first polypeptide comprising an amino acid sequence of SEQ ID NO: 28, a second polypeptide comprising an amino acid sequence of SEQ ID NO: 42 and a third polypeptide comprising an amino acid sequence of SEQ ID NO: 42, and the second antigen binding molecule comprises a first polypeptide comprising an amino acid sequence of SEQ ID NO: 241, a second polypeptide comprising an amino acid sequence of SEQ ID NO: 245 and a third polypeptide comprising an amino acid sequence of SEQ ID NO: 29.

49. The pair of antigen binding molecules according to claim 1, wherein the first and second cytokine receptor-binding domains are Fab molecules or scFv molecules.

50. The pair of antigen binding molecules according to claim 49, wherein
a) the first cytokine receptor-binding domain comprises a VH domain comprising the amino acid sequence of SEQ ID NO: 282 and a VL domain comprising the amino acid sequence of SEQ ID NO: 283 or VH domain comprising the amino acid sequence of SEQ ID NO: 284 and a VL domain comprising the amino acid sequence of SEQ ID NO: 285, and the second cytokine receptor domain comprises a VH domain comprising the amino acid sequence of SEQ ID NO: 286 and a VL domain comprising the amino acid sequence of SEQ ID NO: 287 or VH domain comprising the amino acid sequence of SEQ ID NO: 288 and a VL domain comprising the amino acid sequence of SEQ ID NO: 289; or
b) the first cytokine receptor-binding domain comprises a VH domain comprising the amino acid sequence of SEQ ID NO: 286 and a VL domain comprising the amino acid sequence of SEQ ID NO: 287 or VH domain comprising the amino acid sequence of SEQ ID NO: 288 and a VL domain comprising the amino acid sequence of SEQ ID NO: 289, and the second cytokine receptor domain comprises a VH domain comprising the amino acid sequence of SEQ ID NO: 282 and a VL domain comprising the amino acid sequence of SEQ ID NO: 283 or VH domain comprising the amino acid sequence of SEQ ID NO: 284 and a VL domain comprising the amino acid sequence of SEQ ID NO: 285; or
c) the first cytokine receptor-binding domain comprises a VH domain comprising the amino acid sequence of SEQ ID NO: 290 and a VL domain comprising the amino acid sequence of SEQ ID NO: 291, and the second cytokine receptor domain comprises a VH domain comprising the amino acid sequence of SEQ ID NO: 292 and a VL domain comprising the amino acid sequence of SEQ ID NO: 293; or
d) the first cytokine receptor-binding domain comprises a VH domain comprising the amino acid sequence of SEQ ID NO: 292 and a VL domain comprising the amino acid sequence of SEQ ID NO: 293, and the second cytokine receptor domain comprises a VH domain comprising the amino acid sequence of SEQ ID NO: 290 and a VL domain comprising the amino acid sequence of SEQ ID NO: 291.

51. (canceled)

52. (canceled)

53. The pair of antigen binding molecules according to claim 49, wherein the Fc domains of the first and second antigen binding molecules comprise a first Fc domain subunit and a second Fc domain subunit.

54. The pair of antigen binding molecules according to claim 49, wherein the Fc domains of the first and second antigen binding molecules are IgG, particularly an IgG₁, Fc domains.

55. The pair of antigen binding molecules according to claim 49, wherein the Fc domains of the first and second antigen binding molecules are human Fc domains.

56. The pair of antigen binding molecules according to claim 49, wherein the Fc domains of the first and second antigen binding molecules comprise a modification promoting the association of the first and the second subunit of the Fc domains.

57. The pair of antigen binding molecules according to claim 49, wherein the Fc domains of the first and second antigen binding molecules comprise one or more amino acid substitution that reduces binding to an Fc receptor and/or effector function.

58. The pair of antigen binding molecules according to claim 49, wherein the cytokine receptor-binding domains are fused via peptide linkers to their respective fusion points.

59. The pair of antigen binding molecules according to claim 58, wherein the peptide linkers comprise an amino acid sequence of SEQ ID NO: 280 or SEQ ID NO: 281.

60. The pair of antigen binding molecules according to claim 49, wherein,
a) the first antigen binding molecule comprises a first polypeptide comprising an amino acid sequence of SEQ ID NO: 267, a second polypeptide comprising an amino acid sequence of SEQ ID NO: 28, a third polypeptide comprising an amino acid sequence of SEQ ID NO: 265 and a fourth polypeptide comprising an amino acid sequence of SEQ ID NO: 266, and the second antigen binding molecule comprises a first polypeptide comprising an amino acid sequence of SEQ ID NO: 272, a second polypeptide comprising an amino acid sequence of SEQ ID NO: 28, a third polypeptide comprising an amino acid sequence of SEQ ID NO: 270 and a fourth polypeptide comprising an amino acid sequence of SEQ ID NO: 271; or
b) the first antigen binding molecule comprises a first polypeptide comprising an amino acid sequence of SEQ ID NO: 267, a second polypeptide comprising an amino acid sequence of SEQ ID NO: 28, a third polypeptide comprising an amino acid sequence of SEQ ID NO: 265 and a fourth polypeptide comprising an amino acid sequence of SEQ ID NO: 266, and the second antigen binding molecule comprises a first polypeptide comprising an amino acid sequence of SEQ ID NO: 274, a second polypeptide comprising an amino acid sequence of SEQ ID NO: 28, a third polypeptide comprising an amino acid sequence of SEQ ID NO: 270 and a fourth polypeptide comprising an amino acid sequence of SEQ ID NO: 273; or
c) the first antigen binding molecule comprises a first polypeptide comprising an amino acid sequence of SEQ ID NO: 269, a second polypeptide comprising an amino acid sequence of SEQ ID NO: 28, a third polypeptide comprising an amino acid sequence of SEQ ID NO: 265 and a fourth polypeptide comprising an amino acid sequence of SEQ ID NO: 268, and the second antigen binding molecule comprises a first polypeptide comprising an amino acid sequence of SEQ ID NO: 268, a second polypeptide comprising an amino acid sequence of SEQ ID NO: 28, a third polypeptide comprising an amino acid sequence of SEQ ID NO: 270 and a fourth polypeptide comprising an amino acid sequence of SEQ ID NO: 271; or
d) the first antigen binding molecule comprises a first polypeptide comprising an amino acid sequence of SEQ ID NO: 269, a second polypeptide comprising an amino acid sequence of SEQ ID NO: 28, a third polypeptide comprising an amino acid sequence of SEQ ID NO: 265 and a fourth polypeptide comprising an amino acid sequence of SEQ ID NO: 268, and the second antigen binding molecule comprises a first polypeptide comprising an amino acid sequence of SEQ ID NO: 274, a second polypeptide comprising an amino acid sequence of SEQ ID NO: 28, a third polypeptide comprising an amino acid sequence of SEQ ID NO: 270 and a fourth polypeptide comprising an amino acid sequence of SEQ ID NO: 273.

61. The pair of antigen binding molecules according to claim 49, wherein the first antigen binding molecule comprises a first polypeptide comprising an amino acid sequence of SEQ ID NO: 276, a second polypeptide comprising an amino acid sequence of SEQ ID NO: 28 and a third polypeptide comprising an amino acid sequence of SEQ ID NO: 275, and the second antigen binding molecule comprises a first polypeptide comprising an amino acid sequence of SEQ ID NO: 277, a second polypeptide comprising an amino acid sequence of SEQ ID NO: 28 and a third polypeptide comprising an amino acid sequence of SEQ ID NO: 278.


SEQUENCE LISTING (293 sequences)

SEQ ID NO: 1 - protein, 115 aa, synthetic construct
QVQLQESGGGLVQAGGSLRLSCVASGSLFRWTAMAWYRQAPGKQRELVAVISPAGSIDYADSVKGRFTISRDNGKNVVYLQMNSLKPEDTAVYYCSIHHSARAYWGQGTQVTVSS

SEQ ID NO: 2 - protein, 111 aa, synthetic construct
QVQLQESGGGLVQPGGSLTLSCAASIHLFSRTAMDWYRQAPGRQRELVATISPVGSTYYADSAKDRFTISRDNANNMVYLQMNSLGPEDTAVYYCISGQLQGQGTQVTVSS

SEQ ID NO: 3 - protein, 119 aa, synthetic construct
QVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSS

SEQ ID NO: 4 - protein, 119 aa, synthetic construct
QVQLQQFGGGLVQAGGSLRLSCTASGSTFSFSNYHMGWYRQAPGKQRERVASISSRGSTYYSDSVKGRFAISRDTARNTVYLQMNSLEPEETAVYYCNARGRATGRDYWGQGTLVTVSS

SEQ ID NO: 5 - protein, 122 aa, synthetic construct
QVQLQQFGGGLVQPGGSLRLSCAVSRSIFSIDAMAWYRQAPGKQRNLVGTITSDGTTNYVDSAKGRFTISRDNAKNTVYLQMNSLKPEDTAVYFCNAGAVSRTGGYRPSGYWGQGTLVTVSS

SEQ ID NO: 6 - protein, 119 aa, synthetic construct
QVQLQESGGGLVQAGGSLRLSCTASGAYFIMGWFRQAPGKQRELVARTIRDGTTDYADSVKGRFTISRNTAENTAYLQMNSLKPEDTGVYYCAAGPLSKSFAPWDPAYWGQGTLVTVSS

SEQ ID NO: 7 - protein, 117 aa, synthetic construct
QVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSS

SEQ ID NO: 8 - protein, 123 aa, synthetic construct
QVQLQQFGGGLVQPGGSLTLSCAASGSIFSINPMGWYRQAPGKRRELVASLTNRGITTYADSVKGRFTISRDNAKNTVYLQMNALKPEDTAVYYCHAYVDTGSHEYPQYENYWGQGTLVTVSS

SEQ ID NO: 9 - protein, 111 aa, synthetic construct
QVQLQESGGGLVQAGGSLRLSCAASGSTFTFSTMAWYRQAPGKQRELVATFFSPSTWYADSVEGRFTVSRNNAKNTMYLHMNSLKPEDTAVYYCTSPSASNGQGTQVTVSS

SEQ ID NO: 10 - protein, 123 aa, synthetic construct
QVQLQESGGGLVQPGGSLTLSCAASGSIVSINPMGWYRQAPGKRRELVASLTNRGITTYADSVKGRFTISRDNAKNTVYLQMNTLKPEDTAVYYCHAYVDTGNYEYPQYENYWGQGTLVTVSS

SEQ ID NO: 11 - protein, 369 aa, synthetic construct
QVQLQESGGGLVQAGGSLRLSCVASGSLFRWTAMAWYRQAPGKQRELVAVISPAGSIDYADSVKGRFTISRDNGKNVVYLQMNSLKPEDTAVYYCSIHHSARAYWGQGTQVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 365 aa, synthetic construct
QVQLQESGGGLVQPGGSLTLSCAASIHLFSRTAMDWYRQAPGRQRELVATISPVGSTYYADSAKDRFTISRDNANNMVYLQMNSLGPEDTAVYYCISGQLQGQGTQVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 373 aa, synthetic construct
QVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 367 aa, synthetic construct
QVQLQQFGGGLVQPGGSLRLSCAVSRSIFSIDAMAWYRQAPGKQRNLVGTITSDGTTNYVDSAKGRFTISRDNAKNTVYLQMNSLKPEDTAVYFCNAGAVSRTGGYRPSGYWGQGTLVTVSSTSGSGGGGSGGGGSGGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 373 aa, synthetic construct
QVQLQESGGGLVQAGGSLRLSCTASGAYFIMGWFRQAPGKQRELVARTIRDGTTDYADSVKGRFTISRNTAENTAYLQMNSLKPEDTGVYYCAAGPLSKSFAPWDPAYWGQGTLVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 16 - protein, 371 aa, synthetic construct
QVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 17 - protein, 377 aa, synthetic construct
QVQLQQFGGGLVQPGGSLTLSCAASGSIFSINPMGWYRQAPGKRRELVASLTNRGITTYADSVKGRFTISRDNAKNTVYLQMNALKPEDTAVYYCHAYVDTGSHEYPQYENYWGQGTLVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 18 - protein, 365 aa, synthetic construct
QVQLQESGGGLVQAGGSLRLSCAASGSTFTFSTMAWYRQAPGKQRELVATFFSPSTWYADSVEGRFTVSRNNAKNTMYLHMNSLKPEDTAVYYCTSPSASNGQGTQVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 19 - protein, 377 aa, synthetic construct
QVQLQESGGGLVQPGGSLTLSCAASGSIVSINPMGWYRQAPGKRRELVASLTNRGITTYADSVKGRFTISRDNAKNTVYLQMNTLKPEDTAVYYCHAYVDTGNYEYPQYENYWGQGTLVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 20 - protein, 117 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 21 - protein, 108 aa, synthetic construct
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 22 - protein, 119 aa, synthetic construct
QVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSS

SEQ ID NO: 23 - protein, 111 aa, synthetic construct
EIVLTQSPATLSLSPGERATLSCRASESVDNYGLSFINWFQQKPGQAPRLLIYGTSNRGSGIPARFSGSGSGTDFTLTISSLEPEDFAVYFCQQSNEVPYTFGGGTKVEIK

SEQ ID NO: 24 - protein, 593 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRP

SEQ ID NO: 25 - protein, 215 aa, synthetic construct
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 144 aa, synthetic construct
MQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFQGRRASQ

SEQ ID NO: 27 - protein, 590 aa, synthetic construct
QVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSSGGSGGGGSGGGGSGGGGSGGGGSGGEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 29 - protein, 218 aa, synthetic construct
EIVLTQSPATLSLSPGERATLSCRASESVDNYGLSFINWFQQKPGQAPRLLIYGTSNRGSGIPARFSGSGSGTDFTLTISSLEPEDFAVYFCQQSNEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 590 aa, synthetic construct
QVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSSGGSGGGGSGGGGSGGGGSGGGGSGGQVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 359 aa, synthetic construct
QVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSSGGSGGGGSGGGGSGGGGSGGGGSGGEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 446 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 33 - protein, 360 aa, synthetic construct
QVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSSGGSGGGGSGGGGSGGGGSGGGGSGGEIVLTQSPATLSLSPGERATLSCRASESVDNYGLSFINWFQQKPGQAPRLLIYGTSNRGSGIPARFSGSGSGTDFTLTISSLEPEDFAVYFCQQSNEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 448 aa, synthetic construct
QVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 35 - protein, 357 aa, synthetic construct
QVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSSGGSGGGGSGGGGSGGGGSGGGGSGGEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 362 aa, synthetic construct
QVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSSGGSGGGGSGGGGSGGGGSGGGGSGGEIVLTQSPATLSLSPGERATLSCRASESVDNYGLSFINWFQQKPGQAPRLLIYGTSNRGSGIPARFSGSGSGTDFTLTISSLEPEDFAVYFCQQSNEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 592 aa, synthetic construct
QVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSSGGSGGGGSGGGGSGGGGSGGGGSGGQVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 38 - protein, 588 aa, synthetic construct
QVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSSGGSGGGGSGGGGSGGGGSGGGGSGGEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 39 - protein, 360 aa, synthetic construct
QVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSSGGSGGGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 40 - protein, 358 aa, synthetic construct
QVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSSGGSGGGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 41 - protein, 224 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 573 aa, synthetic construct
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGQVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSS

SEQ ID NO: 43 - protein, 218 aa, synthetic construct
EIVLTQSPATLSLSPGERATLSCRASESVDNYGLSFINWFQQKPGQAPRLLIYGTSNRGSGIPARFSGSGSGTDFTLTISSLEPEDFAVYFCQQSNEVPYTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 45 - protein, 580 aa, synthetic construct
QVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGSGGGGSGGGGSGGQVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSS

SEQ ID NO: 46 - protein, 571 aa, synthetic construct
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGQVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSS

SEQ ID NO: 47 - protein, 582 aa, synthetic construct
QVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGSGGGGSGGGGSGGQVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSS

SEQ ID NO: 48 - protein, 477 aa, synthetic construct
EMGTADLGPSSVPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIVNSSGGGGSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGSGGLNDIFEAQKIEWHE

SEQ ID NO: 49 - protein, 489 aa, synthetic construct
SQLPAPQHPKIRLYNAEQVLSWEPVALSNSTRPVVYQVQFKYTDSKWFTADIMSIGVNCTQITATECDFTAASPSAGFPMDFNVTLRLRAELGALHSAWVTMPWFQHYRNVTVGPPENIEVTPGEGSLIIRFSSPFDIADTSTAFFSYYVHYWEKGGIQQVKGPFRSNSISLDNLKPSRVYCLQVQAQLLWNKSNIFRVGHLSNISCYETMADASTELQQGGGGSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGSGGGSAWSHPQFEKGGGSGGGSGGSAWSHPQFEK

SEQ ID NO: 50 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 51 - protein, 244 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGLNDIFEAQKIEWHE

SEQ ID NO: 52 - protein, 242 aa, synthetic construct
GLNDIFEAQKIEWHEDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 244 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKSGGLNDIFEAQKIEWHE

SEQ ID NO: 54 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 55 - protein, 373 aa, synthetic construct
QVQLQQFGGGLVQAGGSLRLSCTASGSTFSFSNYHMGWYRQAPGKQRERVASISSRGSTYYSDSVKGRFAISRDTARNTVYLQMNSLEPEETAVYYCNARGRATGRDYWGQGTLVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 56 - protein, 117 aa, synthetic construct
QVQLAESGGGLVQPGGSLTLSCVASGSILEMSRMAWYRQAPGKMRELVASITSIGSIVYADSVKGRFTLSRDNTKNTVYLQMNSLKPEDTAVYRCNALGTWSDETYWGQGTQVTVSS

SEQ ID NO: 57 - protein, 117 aa, synthetic construct
QVQLQQFGGGLVQPGGSLTLSCVASESISEMSRMAWYRQAPGKQRELVASITMHGGVVYADAVKGRFTISRDNTKNTVYLQMNSLKPEDTAVYRCNALGTWSDETYWGQGTLVTVSS

SEQ ID NO: 58 - protein, 117 aa, synthetic construct
QVQLQESGGGLVQPGGSLTLSCVASGSILEMSRMAWYRQAPGKQRELVASITSIGSKVYADSVKGRFTLSRDNTKNTIYLQMNSLKPEDTAVYRCNLLGTWSDETYWGQGTLVTVSS

SEQ ID NO: 59 - protein, 121 aa, synthetic construct
QVQLQASGGGLVQSGGSLRLSCVASGSTFSINGMGWYRQAPGKERELVATLTAGGNADYAVSVAGRFIISRGDKKNTRILQMNDLKPEDTAVYYCMADIYTGDSYSGVDYWGKGTLVTVSS

SEQ ID NO: 60 - protein, 117 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSS

SEQ ID NO: 61 - protein, 115 aa, synthetic construct
QVQLQQFGGGSVQAGGSLRLSCSAPGIIFEDIVMGWYRQGPGKQRELVALINSAVTDYADSVKGRFTISRDNAKNLVYLQMNSLRSEDTAVYYCTAIDTNWAQYWGQGTQVTVSS

SEQ ID NO: 62 - protein, 117 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSS

SEQ ID NO: 63 - protein, 120 aa, synthetic construct
QVQLQESGGGLVQPGGSLRLSCAASGSIFSGNAMGWYRQAPGKQRELVASITSGGDTHYVDSVKGRFTISRDNAKNMVYLQMSSLKPEDTAVYYCNAQERVYSDYAFASWGPGTQVTVSS

SEQ ID NO: 64 - protein, 115 aa, synthetic construct
QVQLQESGGGLVQPGGSLRLSCAASGFTFSTYVMNWVRQAPGKGLEWVSGIDSDGDSTTYTDSVKGRFTISRDNAKNTLYLQMNSLKPEDTAVYYCSMFRMATTGSQGTQVTVSS

SEQ ID NO: 65 - protein, 119 aa, synthetic construct
QVQLQESGGGLVQAGGSLRLSCAASGITSSIYAMGWYRQAPGNEREPVALITSGDNTNYPDSVKGRFTISRDTAKNTVYLQMSSLKPEDTAVYYCYADVVIGTTYYTSWGQGTLVTVSS

SEQ ID NO: 66 - protein, 371 aa, synthetic construct
QVQLAESGGGLVQPGGSLTLSCVASGSILEMSRMAWYRQAPGKMRELVASITSIGSIVYADSVKGRFTLSRDNTKNTVYLQMNSLKPEDTAVYRCNALGTWSDETYWGQGTQVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 67 - protein, 371 aa, synthetic construct
QVQLQQFGGGLVQPGGSLTLSCVASESISEMSRMAWYRQAPGKQRELVASITMHGGVVYADAVKGRFTISRDNTKNTVYLQMNSLKPEDTAVYRCNALGTWSDETYWGQGTLVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 68 - protein, 371 aa, synthetic construct
QVQLQESGGGLVQPGGSLTLSCVASGSILEMSRMAWYRQAPGKQRELVASITSIGSKVYADSVKGRFTLSRDNTKNTIYLQMNSLKPEDTAVYRCNLLGTWSDETYWGQGTLVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 69 - protein, 371 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 70 - protein, 371 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 71 - protein, 374 aa, synthetic construct
QVQLQESGGGLVQPGGSLRLSCAASGSIFSGNAMGWYRQAPGKQRELVASITSGGDTHYVDSVKGRFTISRDNAKNMVYLQMSSLKPEDTAVYYCNAQERVYSDYAFASWGPGTQVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 72 - protein, 369 aa, synthetic construct
QVQLQESGGGLVQPGGSLRLSCAASGFTFSTYVMNWVRQAPGKGLEWVSGIDSDGDSTTYTDSVKGRFTISRDNAKNTLYLQMNSLKPEDTAVYYCSMFRMATTGSQGTQVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 73 - protein, 373 aa, synthetic construct
QVQLQESGGGLVQAGGSLRLSCAASGITSSIYAMGWYRQAPGNEREPVALITSGDNTNYPDSVKGRFTISRDTAKNTVYLQMSSLKPEDTAVYYCYADVVIGTTYYTSWGQGTLVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 74 - protein, 366 aa, synthetic construct
QVQLQASGGGLVQSGGSLRLSCVASGSTFSINGMGWYRQAPGKERELVATLTAGGNADYAVSVAGRFIISRGDKKNTRILQMNDLKPEDTAVYYCMADIYTGDSYSGVDYWGKGTLVTVSSTSGSGGGGSGGGGSGGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 75 - protein, 369 aa, synthetic construct
QVQLQQFGGGSVQAGGSLRLSCSAPGIIFEDIVMGWYRQGPGKQRELVALINSAVTDYADSVKGRFTISRDNAKNLVYLQMNSLRSEDTAVYYCTAIDTNWAQYWGQGTQVTVSSTSGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 76 - protein, 120 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSS

SEQ ID NO: 77 - protein, 111 aa, synthetic construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIK

SEQ ID NO: 78 - protein, 124 aa, synthetic construct
EVQLLESGGGLVQPGGSLRLSCAASGFTFSSYAMSWVRQAPGKGLEWVSAITGSGGSTYYADSVKGRFTISRDNSRNTLYLQMNSLRAEDTAVYYCAKGEGYAGSSYFRASDIWGQGTMVTVSS

SEQ ID NO: 79 - protein, 107 aa, synthetic construct
DIQMTQSPSSLSASVGDRVTITCRASQSISSYLNWYQQKPGKAPKLLIYTASSLQSGVPSRFSGSGSGTDFTLTISSLQPEDFATYYCQQSYSTPLTFGGGTKVEIK

SEQ ID NO: 80 - protein, 461 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGAQDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGLNDIFEAQKIEWHE

SEQ ID NO: 81 - protein, 470 aa, synthetic construct
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAGAQDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 82 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 83 - protein, 218 aa, synthetic construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 597 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 85 - protein, 450 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 86 - protein, 594 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSTSGSGGGGSGGGGSGGGGSGGGGSGGGGEVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 87 - protein, 359 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSTSGSGGGGSGGGGSGGGGSGGGGSGGGGDIQMTQSPSSLSASVGDRVTITCRASQSISSYLNWYQQKPGKAPKLLIYTASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 453 aa, synthetic construct
EVQLLESGGGLVQPGGSLRLSCAASGFTFSSYAMSWVRQAPGKGLEWVSAITGSGGSTYYADSVKGRFTISRDNSRNTLYLQMNSLRAEDTAVYYCAKGEGYAGSSYFRASDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 89 - protein, 214 aa, synthetic construct
DIQMTQSPSSLSASVGDRVTITCRASQSISSYLNWYQQKPGKAPKLLIYTASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 598 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSTSGSGGGGSGGGGSGGGGSGGGGSGGGGEVQLLESGGGLVQPGGSLRLSCAASGFTFSSYAMSWVRQAPGKGLEWVSAITGSGGSTYYADSVKGRFTISRDNSRNTLYLQMNSLRAEDTAVYYCAKGEGYAGSSYFRASDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 91 - protein, 363 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSTSGSGGGGSGGGGSGGGGSGGGGSGGGGDIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 449 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 93 - protein, 117 aa, synthetic construct
EVQLLESGGGLVQPGGSLRLSCAASGFTFSSYWMSWVRQAPGKGLEWVSAISGSGGSRYYAESVKGRFTISRDNSKNTLYLQMNSLRAEDTAVYYCASSPLQWIDVWGQGTTVTVSS

SEQ ID NO: 94 - protein, 107 aa, synthetic construct
DIQMTQSPSSLSASVGDRVTITCRASQGISSWLAWYQQKPGKAPKLLIYEASSLQSGVPSRFSGSGSGTDFTLTISSLQPEDFATYYCQQANQFPFTFGPGTKVDIK

SEQ ID NO: 95 - protein, 356 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSGGSGGGGSGGGGSGGGGSGGGGSGGDIQMTQSPSSLSASVGDRVTITCRASQGISSWLAWYQQKPGKAPKLLIYEASSLQSGVPSRFSGSGSGTDFTLTISSLQPEDFATYYCQQANQFPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 446 aa, synthetic construct
EVQLLESGGGLVQPGGSLRLSCAASGFTFSSYWMSWVRQAPGKGLEWVSAISGSGGSRYYAESVKGRFTISRDNSKNTLYLQMNSLRAEDTAVYYCASSPLQWIDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 97 - protein, 356 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSGGSGGGGSGGGGSGGGGSGGGGSGGDIQMTQSPSSLSASVGDRVTITCRASQGISSWLAWYQQKPGKAPKLLIYEASSLQSGVPSRFSGSGSGTDFTLTISSLQPEDFATYYCQQANQFPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 214 aa, synthetic construct
DIQMTQSPSSLSASVGDRVTITCRASQGISSWLAWYQQKPGKAPKLLIYEASSLQSGVPSRFSGSGSGTDFTLTISSLQPEDFATYYCQQANQFPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 588 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSGGSGGGGSGGGGSGGGGSGGGGSGGEVQLLESGGGLVQPGGSLRLSCAASGFTFSSYWMSWVRQAPGKGLEWVSAISGSGGSRYYAESVKGRFTISRDNSKNTLYLQMNSLRAEDTAVYYCASSPLQWIDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 100 - protein, 588 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSGGSGGGGSGGGGSGGGGSGGGGSGGEVQLLESGGGLVQPGGSLRLSCAASGFTFSSYWMSWVRQAPGKGLEWVSAISGSGGSRYYAESVKGRFTISRDNSKNTLYLQMNSLRAEDTAVYYCASSPLQWIDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 101 - protein, 595 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSGGSGGGGSGGGGSGGGGSGGGGSGGEVQLLESGGGLVQPGGSLRLSCAASGFTFSSYAMSWVRQAPGKGLEWVSAITGSGGSTYYADSVKGRFTISRDNSRNTLYLQMNSLRAEDTAVYYCAKGEGYAGSSYFRASDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 102 - protein, 356 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSGGSGGGGSGGGGSGGGGSGGGGSGGDIQMTQSPSSLSASVGDRVTITCRASQSISSYLNWYQQKPGKAPKLLIYTASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 595 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSGGSGGGGSGGGGSGGGGSGGGGSGGEVQLLESGGGLVQPGGSLRLSCAASGFTFSSYAMSWVRQAPGKGLEWVSAITGSGGSTYYADSVKGRFTISRDNSRNTLYLQMNSLRAEDTAVYYCAKGEGYAGSSYFRASDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 104 - protein, 356 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSGGSGGGGSGGGGSGGGGSGGGGSGGDIQMTQSPSSLSASVGDRVTITCRASQSISSYLNWYQQKPGKAPKLLIYTASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 357 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSGGSGGGGSGGGGSGGGGSGGGGSGGEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 226 aa, synthetic construct
DKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNAYTQKSLSLSPG

SEQ ID NO: 107 - protein, 357 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSGGSGGGGSGGGGSGGGGSGGGGSGGEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 360 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSGGSGGGGSGGGGSGGGGSGGGGSGGEIVLTQSPATLSLSPGERATLSCRASESVDNYGLSFINWFQQKPGQAPRLLIYGTSNRGSGIPARFSGSGSGTDFTLTISSLEPEDFAVYFCQQSNEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 360 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSGGSGGGGSGGGGSGGGGSGGGGSGGEIVLTQSPATLSLSPGERATLSCRASESVDNYGLSFINWFQQKPGQAPRLLIYGTSNRGSGIPARFSGSGSGTDFTLTISSLEPEDFAVYFCQQSNEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 588 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSGGSGGGGSGGGGSGGGGSGGGGSGGEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 111 - protein, 588 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSGGSGGGGSGGGGSGGGGSGGGGSGGEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 112 - protein, 590 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSGGSGGGGSGGGGSGGGGSGGGGSGGQVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 113 - protein, 590 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSGGSGGGGSGGGGSGGGGSGGGGSGGQVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 114 - protein, 120 aa, synthetic construct [sequence omitted]

SEQ ID NO: 115 - protein, 106 aa, synthetic construct [sequence omitted]

SEQ ID NO: 116 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 117 - protein, 107 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 118 - protein, 355 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSGGSGGGGSGGGGSGGGGSGGGGSGGDIQMTQSPSSLSASVGDRVTITCRASQGINNYLNWYQQKPGKAPKRLIYNTNNLQTGVPSRFSGSGSGTEFTLTISSLQPEDFATYYCLQHNSFPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 449 aa, synthetic construct
QVQLVQSGAEVKKPGSSVKVSCKASGFTFTDYKIHWVRQAPGQGLEWMGYFNPNSGYSTYAQKFQGRVTITADKSTSTAYMELSSLRSEDTAVYYCARLSPGGYYVMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 120 - protein, 355 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSGGSGGGGSGGGGSGGGGSGGGGSGGDIQMTQSPSSLSASVGDRVTITCRASQGINNYLNWYQQKPGKAPKRLIYNTNNLQTGVPSRFSGSGSGTEFTLTISSLQPEDFATYYCLQHNSFPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 356 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSGGSGGGGSGGGGSGGGGSGGGGSGGDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - protein, 448 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 123 - protein, 356 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSGGSGGGGSGGGGSGGGGSGGGGSGGDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 124 - protein, 213 aa, synthetic construct
DIQMTQSPSSLSASVGDRVTITCRASQGINNYLNWYQQKPGKAPKRLIYNTNNLQTGVPSRFSGSGSGTEFTLTISSLQPEDFATYYCLQHNSFPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - protein, 591 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSGGSGGGGSGGGGSGGGGSGGGGSGGQVQLVQSGAEVKKPGSSVKVSCKASGFTFTDYKIHWVRQAPGQGLEWMGYFNPNSGYSTYAQKFQGRVTITADKSTSTAYMELSSLRSEDTAVYYCARLSPGGYYVMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 126 - protein, 591 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSGGSGGGGSGGGGSGGGGSGGGGSGGQVQLVQSGAEVKKPGSSVKVSCKASGFTFTDYKIHWVRQAPGQGLEWMGYFNPNSGYSTYAQKFQGRVTITADKSTSTAYMELSSLRSEDTAVYYCARLSPGGYYVMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 127 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - protein, 590 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSGGSGGGGSGGGGSGGGGSGGGGSGGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 129 - protein, 590 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSGGSGGGGSGGGGSGGGGSGGGGSGGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 130 - protein, 120 aa, synthetic construct [sequence omitted]

SEQ ID NO: 131 - protein, 107 aa, synthetic construct [sequence omitted]

SEQ ID NO: 132 - protein, 119 aa, synthetic construct [sequence omitted]

SEQ ID NO: 133 - protein, 107 aa, synthetic construct [sequence omitted]

SEQ ID NO: 134 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 135 - protein, 591 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSGGSGGGGSGGGGSGGGGSGGGGSGGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 136 - protein, 591 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSGGSGGGGSGGGGSGGGGSGGGGSGGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 137 - protein, 356 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSGGSGGGGSGGGGSGGGGSGGGGSGGDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - protein, 448 aa, synthetic construct
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 139 - protein, 356 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSGGSGGGGSGGGGSGGGGSGGGGSGGDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 140 - protein, 120 aa, synthetic construct
QVQLVQSGAEVKKPGASVKVSCKASGYTFRSSYISWVRQAPGQGLEWMGWIYAGTGSPSYNQKLQGRVTMTTDTSTSTAYMELRSLRSDDTAVYYCARHRDYYSNSLTYWGQGTLVTVSS

SEQ ID NO: 141 - protein, 113 aa, synthetic construct
DIVMTQSPDSLAVSLGERATINCKSSQSVLNSGNQKNYLTWYQQKPGQPPKLLIYWASTRESGVPDRFSGSGSGTDFTLTISSLQAEDVAVYYCQSDYSYPYTFGQGTKLEIK

SEQ ID NO: 142 - protein, 116 aa, synthetic construct
QVQLQQSGAELAKPGASVKMSCKTSGYTLTDYWIHWVKQRPGQGLEWIGYINPYTGYTESNQKFKDKATLTADKSSNTAYIQLSSLTSEDSAVYYCARPYYYGDYWGQGTTLTVSS

SEQ ID NO: 143 - protein, 106 aa, synthetic construct
QIVLTQSPAIMSASPGERVTITCSASSSVSYMHWFQQKPGTSPKLLIYSTSNLASGVPARFSGSGSGTSYSLTISRMEAEDAATYYCQQRSSYPFTFGSGTKLEIK

SEQ ID NO: 144 - protein, 220 aa, synthetic construct
DIVMTQSPDSLAVSLGERATINCKSSQSVLNSGNQKNYLTWYQQKPGQPPKLLIYWASTRESGVPDRFSGSGSGTDFTLTISSLQAEDVAVYYCQSDYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - protein, 591 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSGGSGGGGSGGGGSGGGGSGGGGSGGQVQLVQSGAEVKKPGASVKVSCKASGYTFRSSYISWVRQAPGQGLEWMGWIYAGTGSPSYNQKLQGRVTMTTDTSTSTAYMELRSLRSDDTAVYYCARHRDYYSNSLTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 146 - protein, 591 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSGGSGGGGSGGGGSGGGGSGGGGSGGQVQLVQSGAEVKKPGASVKVSCKASGYTFRSSYISWVRQAPGQGLEWMGWIYAGTGSPSYNQKLQGRVTMTTDTSTSTAYMELRSLRSDDTAVYYCARHRDYYSNSLTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 147 - protein, 355 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSGGSGGGGSGGGGSGGGGSGGGGSGGQIVLTQSPAIMSASPGERVTITCSASSSVSYMHWFQQKPGTSPKLLIYSTSNLASGVPARFSGSGSGTSYSLTISRMEAEDAATYYCQQRSSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 148 - protein, 445 aa, synthetic construct
QVQLQQSGAELAKPGASVKMSCKTSGYTLTDYWIHWVKQRPGQGLEWIGYINPYTGYTESNQKFKDKATLTADKSSNTAYIQLSSLTSEDSAVYYCARPYYYGDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 149 - protein, 355 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSGGSGGGGSGGGGSGGGGSGGGGSGGQIVLTQSPAIMSASPGERVTITCSASSSVSYMHWFQQKPGTSPKLLIYSTSNLASGVPARFSGSGSGTSYSLTISRMEAEDAATYYCQQRSSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 150 - protein, 121 aa, synthetic construct
EVQLLESGGGLVQPGGSLRLSCAASGFIFDDYTMNWVRQAPGKGLEWVAVISWDGGGTYYTDSVKGRFTISRDDFKNTLYLQMNSLRAEDTAVYYCAKGLTDTTLYGSDYWGQGTLVTVSS

SEQ ID NO: 151 - protein, 107 aa, synthetic construct
DIQMTQSPSSLSASVGDRVTITCRASQSISSYLNWYQQKPGKAPKLLIYAASTLQSGVPSRFSGSGSGTDFTLTISSLQPEDFATYYCQQTYSSPLTFGGGTKVEIK

SEQ ID NO: 152 - protein, 122 aa, synthetic construct
EVQLVESGGGLVQPGGSLRLACAASGFTFSDYAMSWVRQAPGKGLEWVSGIDNSGYYTYYTDSVKGRFTISRDDVKNTLYLQMNSLRAEDTAVYLCTKTHSGLIVNDAFDIWGQGTMVTVSS

SEQ ID NO: 153 - protein, 107 aa, synthetic construct
DIQLTQSPSSLSASVGDRVTITCRASQSISSYLNWYQQKPGKAPKLLIYDASSLESGVPSRFSGSGSGTDATLTISSLQPEDFATYYCQQSYSTPLTFGGGTKVEIK

SEQ ID NO: 154 - protein, 356 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSGGSGGGGSGGGGSGGGGSGGGGSGGDIQMTQSPSSLSASVGDRVTITCRASQSISSYLNWYQQKPGKAPKLLIYAASTLQSGVPSRFSGSGSGTDFTLTISSLQPEDFATYYCQQTYSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 155 - protein, 450 aa, synthetic construct
EVQLLESGGGLVQPGGSLRLSCAASGFIFDDYTMNWVRQAPGKGLEWVAVISWDGGGTYYTDSVKGRFTISRDDFKNTLYLQMNSLRAEDTAVYYCAKGLTDTTLYGSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 156 - protein, 356 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSGGSGGGGSGGGGSGGGGSGGGGSGGDIQMTQSPSSLSASVGDRVTITCRASQSISSYLNWYQQKPGKAPKLLIYAASTLQSGVPSRFSGSGSGTDFTLTISSLQPEDFATYYCQQTYSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 157 - protein, 356 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSGGSGGGGSGGGGSGGGGSGGGGSGGDIQLTQSPSSLSASVGDRVTITCRASQSISSYLNWYQQKPGKAPKLLIYDASSLESGVPSRFSGSGSGTDATLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 158 - protein, 451 aa, synthetic construct
EVQLVESGGGLVQPGGSLRLACAASGFTFSDYAMSWVRQAPGKGLEWVSGIDNSGYYTYYTDSVKGRFTISRDDVKNTLYLQMNSLRAEDTAVYLCTKTHSGLIVNDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 159 - protein, 356 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSGGSGGGGSGGGGSGGGGSGGGGSGGDIQLTQSPSSLSASVGDRVTITCRASQSISSYLNWYQQKPGKAPKLLIYDASSLESGVPSRFSGSGSGTDATLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 160 - protein, 214 aa, synthetic construct
DIQMTQSPSSLSASVGDRVTITCRASQSISSYLNWYQQKPGKAPKLLIYAASTLQSGVPSRFSGSGSGTDFTLTISSLQPEDFATYYCQQTYSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 161 - protein, 592 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSGGSGGGGSGGGGSGGGGSGGGGSGGEVQLLESGGGLVQPGGSLRLSCAASGFIFDDYTMNWVRQAPGKGLEWVAVISWDGGGTYYTDSVKGRFTISRDDFKNTLYLQMNSLRAEDTAVYYCAKGLTDTTLYGSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 162 - protein, 592 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSGGSGGGGSGGGGSGGGGSGGGGSGGEVQLLESGGGLVQPGGSLRLSCAASGFIFDDYTMNWVRQAPGKGLEWVAVISWDGGGTYYTDSVKGRFTISRDDFKNTLYLQMNSLRAEDTAVYYCAKGLTDTTLYGSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 163 - protein, 214 aa, synthetic construct
DIQLTQSPSSLSASVGDRVTITCRASQSISSYLNWYQQKPGKAPKLLIYDASSLESGVPSRFSGSGSGTDATLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 164 - protein, 593 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSGGSGGGGSGGGGSGGGGSGGGGSGGEVQLVESGGGLVQPGGSLRLACAASGFTFSDYAMSWVRQAPGKGLEWVSGIDNSGYYTYYTDSVKGRFTISRDDVKNTLYLQMNSLRAEDTAVYLCTKTHSGLIVNDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 165 - protein, 593 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSGGSGGGGSGGGGSGGGGSGGGGSGGEVQLVESGGGLVQPGGSLRLACAASGFTFSDYAMSWVRQAPGKGLEWVSGIDNSGYYTYYTDSVKGRFTISRDDVKNTLYLQMNSLRAEDTAVYLCTKTHSGLIVNDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 166 - protein, 124 aa, synthetic construct
QVQLVESGGGLVQAGGSLALSCAASGRAFGSYNMAWFRQAPGKEREYVAVISGSDSARHYAASAKDRFTISRDNAKNTVYLQMNSLRPEDTAVYYCAADGAAFIVYRAADYDYWGQGTQVTVSS

SEQ ID NO: 167 - protein, 127 aa, synthetic construct
QVQLVESGGGLVQAGGSLRLSCAASGWTHSRYVMGWFRQAPGKEREFVAAISWSGGNTVYADSVKGRFTISRDNAKNTVYLQMNSLKPEDTADYYCAARSSYADAAYYTQSPQYADWGQGTQVTVSS

SEQ ID NO: 168 - protein, 124 aa, synthetic construct
QVQLVESGGGLVQAGGSLRLSCAASGLTLSTYNMGWFRQAPGKEREYVAVISGSDSARHYAASAKDRFTISRDNAKNTVYLQMNSLKPEDTAIYYCAADAAGFIVYGAADYDYWGQGTQVTVSS

SEQ ID NO: 169 - protein, 127 aa, synthetic construct
QVQLVESGGGLVQAGGSLRLSCAASGWTHSRYVMGWFRQAPGKEREFVAAISWSGGNTVYADSVKGRFTISRDNAKNTVYLQMNSLEPDDTADYYCAARSSYADAAYYTQSPQYADWGQGTQVTVSS

SEQ ID NO: 170 - protein, 505 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSGGSGGGGSGGGGSGGGGSGGGGSGGQVQLVESGGGLVQAGGSLRLSCAASGWTHSRYVMGWFRQAPGKEREFVAAISWSGGNTVYADSVKGRFTISRDNAKNTVYLQMNSLEPDDTADYYCAARSSYADAAYYTQSPQYADWGQGTQVTVSSGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 171 - protein, 505 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSGGSGGGGSGGGGSGGGGSGGGGSGGQVQLVESGGGLVQAGGSLRLSCAASGWTHSRYVMGWFRQAPGKEREFVAAISWSGGNTVYADSVKGRFTISRDNAKNTVYLQMNSLKPEDTADYYCAARSSYADAAYYTQSPQYADWGQGTQVTVSSGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 172 - protein, 502 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSGGSGGGGSGGGGSGGGGSGGGGSGGQVQLVESGGGLVQAGGSLALSCAASGRAFGSYNMAWFRQAPGKEREYVAVISGSDSARHYAASAKDRFTISRDNAKNTVYLQMNSLRPEDTAVYYCAADGAAFIVYRAADYDYWGQGTQVTVSSGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 173 - protein, 502 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSGGSGGGGSGGGGSGGGGSGGGGSGGQVQLVESGGGLVQAGGSLALSCAASGRAFGSYNMAWFRQAPGKEREYVAVISGSDSARHYAASAKDRFTISRDNAKNTVYLQMNSLRPEDTAVYYCAADGAAFIVYRAADYDYWGQGTQVTVSSGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 174 - protein, 502 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSGGSGGGGSGGGGSGGGGSGGGGSGGQVQLVESGGGLVQAGGSLRLSCAASGLTLSTYNMGWFRQAPGKEREYVAVISGSDSARHYAASAKDRFTISRDNAKNTVYLQMNSLKPEDTAIYYCAADAAGFIVYGAADYDYWGQGTQVTVSSGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 175 - protein, 502 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSGGSGGGGSGGGGSGGGGSGGGGSGGQVQLVESGGGLVQAGGSLRLSCAASGLTLSTYNMGWFRQAPGKEREYVAVISGSDSARHYAASAKDRFTISRDNAKNTVYLQMNSLKPEDTAIYYCAADAAGFIVYGAADYDYWGQGTQVTVSSGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 176 - protein, 505 aa, synthetic construct
QVQLQQSGGGLVQPGGSLTLSCVASGSILEMSRMAWYRQAPGKMRELVASITSIGSIVYADSVKGRFTLSRDNTKNTVYLQMNSLKPEDTAVYRCNLLGTWSDETYWGQGTQVTVSSGGSGGGGSGGGGSGGGGSGGGGSGGQVQLVESGGGLVQAGGSLRLSCAASGWTHSRYVMGWFRQAPGKEREFVAAISWSGGNTVYADSVKGRFTISRDNAKNTVYLQMNSLKPEDTADYYCAARSSYADAAYYTQSPQYADWGQGTQVTVSSGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 177 - protein, 117 aa, synthetic construct
QVQLVESGGGLVQPGGSLRLSCAASGFTFSQRIMSWYRQAPGKERELVASISPFGDGTRYADSVKGRFTISRDNARNQLYLQMNTLKPEDTAVYYCNKRNLEISDDWGQGTQVTVSS

SEQ ID NO: 178 - protein, 113 aa, synthetic construct
QVQLVESGGGLVQPGGSLRLSCEVSGIRFSLRNMHWYRQASGRQREWVATITAGGVTGYRDSVKGRFTISRDNAKNTVDLQMNGLQLEDTAVYYCNHGDTAYWGQGTQVTVSS

SEQ ID NO: 179 - protein, 121 aa, synthetic construct
QVQLVESGGGSVQPGGSLRLSCATSGSIFSLATMTWYRQGPGKTRELVAVITWRGTTTYADSVKGRFTISRDNAKNTMDLQMNGLKPEDTAVYYCNVKWGGGLNREPFDYWGQGTQVTVSS

SEQ ID NO: 180 - protein, 117 aa, synthetic construct
QVQLVESGGGLVQPGGSLRLSCTTSGFTFTTYAMKWVRQAPGKGLEWVAFISPAGGITDYADSVKGRFTISRDNAKETLYLRMDSLKPEDTAVYYCARGDSDTNNNRGQGTQVTVSS

SEQ ID NO: 181 - protein, 117 aa, synthetic construct
QVQLVESGGGLVQPGGSLRLSCAASGFTFSQRIMSWYRQAPGKEHELVASISPFGDGTRYADSVKGRFTISRDNARNQLYLQMNSLEFEDTAVYYCNKRNLDVSDDWGQGTQVTVSS

SEQ ID NO: 182 - protein, 121 aa, synthetic construct
QVQLVESGGGSVQPGGSLRLSCATSGSIFSLATMTWYRQGPGKTRELVAVITWRGTTTYADSVKGRFTISRDNAKNTMDLQMNGLKPEDTAVYYCNVKWGGGLNRAPFDYWGQGTQVTVSS

SEQ ID NO: 183 - protein, 115 aa, synthetic construct
QVQLVESGGGLVQPGGSLRLSCAASGFTFSNYAMSWYRQAPGKERELVASISVFGGGTNYADSVKGRFTISRDNAKNTVYLQMNSLKPEDSAVYYCNKRRYDDYWGQGTQVTVSS

SEQ ID NO: 184 - protein, 114 aa, synthetic construct
QVQLVESGGGLVQPGGSLRLSCAASGFTISSWSMKWVRQAPGKGPEWVSYISPGGGSLDYLDSVKGRFTISRDNAKNTVYLQMSNLKSEDTALYWCATGTGPTRGQGTQVTVSS

SEQ ID NO: 185 - protein, 114 aa, synthetic construct
QVQLVESGGGLVQPGGSLRLSCAASGFTISTWSMKWVRQAPGKGPEWVSYISPGGTSLDYQDSVKGRFTISRDNAKNTVYLQMNNLKSEDTALYWCATGTGETRGQGTQVTVSS

SEQ ID NO: 186 - protein, 121 aa, synthetic construct
QVQLVESGGGLVQPGGSLRLSCATSGSIFSLATMTWYRQAPGKTREFVAAITWRGAATYAESVKGRFTISRDNAKNTMDLQMDGLKPEDTAVYYCNVKWGGGLNRAPFDYWGQGTQVTVSS

SEQ ID NO: 187 - protein, 114 aa, synthetic construct
QVQLVESGGGLVQPGGSLRLSCEASGFTISSWSMKWVRQAPGKGPEWVSYISPGGGSLDYHHSVKGRFTISRDNARNTVYLQMSNLQSEDTALYWCATGTGETRGQGTQVTVSS

SEQ ID NO: 188 - protein, 114 aa, synthetic construct
QVQLVESGGGLVQPGGSLRLSCAASGFTISSRSMKWVRQAPGKGPEWVSYISPGGGTVDYQDSVKGRFTISRDNAKNTVYLQMSNLKSEDTALYWCATGTGPTRGRGTQVTVSS

SEQ ID NO: 189 - protein, 113 aa, synthetic construct
QVQLVESGGGLVRPGGSLRLACAASGIRFSLRNMHWYRQASGRQREWVATITAGGVTGYRDSVKGRFTISRDNAKNTVDLQMNGLQLEDTAVYYCNHGDTAYWGQGTQVTVSS

SEQ ID NO: 190 - protein, 117 aa, synthetic construct
QVQLVESGGGLVQPGGSLRLSCAASGFTFSQRIMSWYRQAPGKERELVASVSPFGDGTRYADSVKGRFTISRDNAKSQLYLQMNNLKFEDTAVYYCNKRNLEISDAWGQGTQVTVSS

SEQ ID NO: 191 - protein, 114 aa, synthetic construct
QVQLVESGGGLVQPGGSLRLSCAASGFTISSWSMKWVRQAPGKGPEWVSYISPGGGSLDYHHSVKGRFTISRDNARNTVYLQMSNLQSEDTALYWCATGTGETRGQGTQVTVSS

SEQ ID NO: 192 - protein, 121 aa, synthetic construct
QVQLVESGGGLVQPGGSLRLSCTASGSIFSLATMTWYRQAPGKTREFVAAITWRGAATYAESVKGRFTISRDNAKNTMDLQMDGLKPEDTAVYYCNVKWGGGLNRAPFDYWGQGTQVTVSS

SEQ ID NO: 193 - protein, 360 aa, synthetic construct
QVQLVESGGGLVQPGGSLRLSCAASGFTFSQRIMSWYRQAPGKERELVASISPFGDGTRYADSVKGRFTISRDNARNQLYLQMNTLKPEDTAVYYCNKRNLEISDDWGQGTQVTVSSGGSGGGGSGGGGSGGGGSGGGGSGGDIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 194 - protein, 449 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 195 - protein, 356 aa, synthetic construct
QVQLVESGGGLVQPGGSLRLSCEVSGIRFSLRNMHWYRQASGRQREWVATITAGGVTGYRDSVKGRFTISRDNAKNTVDLQMNGLQLEDTAVYYCNHGDTAYWGQGTQVTVSSGGSGGGGSGGGGSGGGGSGGGGSGGDIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 196 - protein, 364 aa, synthetic construct
QVQLVESGGGSVQPGGSLRLSCATSGSIFSLATMTWYRQGPGKTRELVAVITWRGTTTYADSVKGRFTISRDNAKNTMDLQMNGLKPEDTAVYYCNVKWGGGLNREPFDYWGQGTQVTVSSGGSGGGGSGGGGSGGGGSGGGGSGGDIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 197 - protein, 360 aa, synthetic construct
QVQLVESGGGLVQPGGSLRLSCAASGFTFSQRIMSWYRQAPGKEHELVASISPFGDGTRYADSVKGRFTISRDNARNQLYLQMNSLEFEDTAVYYCNKRNLDVSDDWGQGTQVTVSSGGSGGGGSGGGGSGGGGSGGGGSGGDIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 198 - protein, 364 aa, synthetic construct
QVQLVESGGGSVQPGGSLRLSCATSGSIFSLATMTWYRQGPGKTRELVAVITWRGTTTYADSVKGRFTISRDNAKNTMDLQMNGLKPEDTAVYYCNVKWGGGLNRAPFDYWGQGTQVTVSSGGSGGGGSGGGGSGGGGSGGGGSGGDIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 199 - protein, 358 aa, synthetic construct
QVQLVESGGGLVQPGGSLRLSCAASGFTFSNYAMSWYRQAPGKERELVASISVFGGGTNYADSVKGRFTISRDNAKNTVYLQMNSLKPEDSAVYYCNKRRYDDYWGQGTQVTVSSGGSGGGGSGGGGSGGGGSGGGGSGGDIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 200 - protein, 364 aa, synthetic construct
QVQLVESGGGLVQPGGSLRLSCATSGSIFSLATMTWYRQAPGKTREFVAAITWRGAATYAESVKGRFTISRDNAKNTMDLQMDGLKPEDTAVYYCNVKWGGGLNRAPFDYWGQGTQVTVSSGGSGGGGSGGGGSGGGGSGGGGSGGDIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 201 - protein, 357 aa, synthetic construct
QVQLVESGGGLVQPGGSLRLSCEASGFTISSWSMKWVRQAPGKGPEWVSYISPGGGSLDYHHSVKGRFTISRDNARNTVYLQMSNLQSEDTALYWCATGTGETRGQGTQVTVSSGGSGGGGSGGGGSGGGGSGGGGSGGDIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 202 - protein, 356 aa, synthetic construct
QVQLVESGGGLVRPGGSLRLACAASGIRFSLRNMHWYRQASGRQREWVATITAGGVTGYRDSVKGRFTISRDNAKNTVDLQMNGLQLEDTAVYYCNHGDTAYWGQGTQVTVSSGGSGGGGSGGGGSGGGGSGGGGSGGDIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 203 - protein, 360 aa, synthetic construct
QVQLVESGGGLVQPGGSLRLSCAASGFTFSQRIMSWYRQAPGKERELVASVSPFGDGTRYADSVKGRFTISRDNAKSQLYLQMNNLKFEDTAVYYCNKRNLEISDAWGQGTQVTVSSGGSGGGGSGGGGSGGGGSGGGGSGGDIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 204 - protein, 357 aa, synthetic construct
QVQLVESGGGLVQPGGSLRLSCAASGFTISSWSMKWVRQAPGKGPEWVSYISPGGGSLDYHHSVKGRFTISRDNARNTVYLQMSNLQSEDTALYWCATGTGETRGQGTQVTVSSGGSGGGGSGGGGSGGGGSGGGGSGGDIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 205 - protein, 364 aa, synthetic construct
QVQLVESGGGLVQPGGSLRLSCTASGSIFSLATMTWYRQAPGKTREFVAAITWRGAATYAESVKGRFTISRDNAKNTMDLQMDGLKPEDTAVYYCNVKWGGGLNRAPFDYWGQGTQVTVSSGGSGGGGSGGGGSGGGGSGGGGSGGDIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 206 - protein, 594 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 207 - protein, 447 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 208 - protein, 467 aa, synthetic construct
ESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDASGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGLNDIFEAQKIEWHE

SEQ ID NO: 209 - protein, 226 aa, synthetic construct; identical to SEQ ID NO: 50

SEQ ID NO: 210 - protein, 368 aa, synthetic construct
QVQLVESGGGLVQPGGSLRLSCAASGFTFSQRIMSWYRQAPGKERELVASISPFGDGTRYADSVKGRFTISRDNARNQLYLQMNTLKPEDTAVYYCNKRNLEISDDWGQGTQVTVSSGGSGGGGSGGGGSGGGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 211 - protein, 364 aa, synthetic construct
QVQLVESGGGLVQPGGSLRLSCEVSGIRFSLRNMHWYRQASGRQREWVATITAGGVTGYRDSVKGRFTISRDNAKNTVDLQMNGLQLEDTAVYYCNHGDTAYWGQGTQVTVSSGGSGGGGSGGGGSGGGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 212 - protein, 372 aa, synthetic construct
QVQLVESGGGSVQPGGSLRLSCATSGSIFSLATMTWYRQGPGKTRELVAVITWRGTTTYADSVKGRFTISRDNAKNTMDLQMNGLKPEDTAVYYCNVKWGGGLNREPFDYWGQGTQVTVSSGGSGGGGSGGGGSGGGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 213 - protein, 368 aa, synthetic construct
QVQLVESGGGLVQPGGSLRLSCTTSGFTFTTYAMKWVRQAPGKGLEWVAFISPAGGITDYADSVKGRFTISRDNAKETLYLRMDSLKPEDTAVYYCARGDSDTNNNRGQGTQVTVSSGGSGGGGSGGGGSGGGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 214 - protein, 368 aa, synthetic construct
QVQLVESGGGLVQPGGSLRLSCAASGFTFSQRIMSWYRQAPGKEHELVASISPFGDGTRYADSVKGRFTISRDNARNQLYLQMNSLEFEDTAVYYCNKRNLDVSDDWGQGTQVTVSSGGSGGGGSGGGGSGGGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 215 - protein, 372 aa, synthetic construct
QVQLVESGGGSVQPGGSLRLSCATSGSIFSLATMTWYRQGPGKTRELVAVITWRGTTTYADSVKGRFTISRDNAKNTMDLQMNGLKPEDTAVYYCNVKWGGGLNRAPFDYWGQGTQVTVSSGGSGGGGSGGGGSGGGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 216 - protein, 366 aa, synthetic construct
QVQLVESGGGLVQPGGSLRLSCAASGFTFSNYAMSWYRQAPGKERELVASISVFGGGTNYADSVKGRFTISRDNAKNTVYLQMNSLKPEDSAVYYCNKRRYDDYWGQGTQVTVSSGGSGGGGSGGGGSGGGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 217 - protein, 365 aa, synthetic construct
QVQLVESGGGLVQPGGSLRLSCAASGFTISSWSMKWVRQAPGKGPEWVSYISPGGGSLDYLDSVKGRFTISRDNAKNTVYLQMSNLKSEDTALYWCATGTGPTRGQGTQVTVSSGGSGGGGSGGGGSGGGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 218 - protein, 365 aa, synthetic construct
QVQLVESGGGLVQPGGSLRLSCAASGFTISTWSMKWVRQAPGKGPEWVSYISPGGTSLDYQDSVKGRFTISRDNAKNTVYLQMNNLKSEDTALYWCATGTGETRGQGTQVTVSSGGSGGGGSGGGGSGGGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 219 - protein, 372 aa, synthetic construct
QVQLVESGGGLVQPGGSLRLSCATSGSIFSLATMTWYRQAPGKTREFVAAITWRGAATYAESVKGRFTISRDNAKNTMDLQMDGLKPEDTAVYYCNVKWGGGLNRAPFDYWGQGTQVTVSSGGSGGGGSGGGGSGGGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 220 - protein, 365 aa, synthetic construct
QVQLVESGGGLVQPGGSLRLSCEASGFTISSWSMKWVRQAPGKGPEWVSYISPGGGSLDYHHSVKGRFTISRDNARNTVYLQMSNLQSEDTALYWCATGTGETRGQGTQVTVSSGGSGGGGSGGGGSGGGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 221 - protein, 365 aa, synthetic construct
QVQLVESGGGLVQPGGSLRLSCAASGFTISSRSMKWVRQAPGKGPEWVSYISPGGGTVDYQDSVKGRFTISRDNAKNTVYLQMSNLKSEDTALYWCATGTGPTRGRGTQVTVSSGGSGGGGSGGGGSGGGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 222 - protein, 364 aa, synthetic construct
QVQLVESGGGLVRPGGSLRLACAASGIRFSLRNMHWYRQASGRQREWVATITAGGVTGYRDSVKGRFTISRDNAKNTVDLQMNGLQLEDTAVYYCNHGDTAYWGQGTQVTVSSGGSGGGGSGGGGSGGGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 223 - protein, 368 aa, synthetic construct
QVQLVESGGGLVQPGGSLRLSCAASGFTFSQRIMSWYRQAPGKERELVASVSPFGDGTRYADSVKGRFTISRDNAKSQLYLQMNNLKFEDTAVYYCNKRNLEISDAWGQGTQVTVSSGGSGGGGSGGGGSGGGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 224 - protein, 365 aa, synthetic construct
QVQLVESGGGLVQPGGSLRLSCAASGFTISSWSMKWVRQAPGKGPEWVSYISPGGGSLDYHHSVKGRFTISRDNARNTVYLQMSNLQSEDTALYWCATGTGETRGQGTQVTVSSGGSGGGGSGGGGSGGGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 225 - protein, 372 aa, synthetic construct
QVQLVESGGGLVQPGGSLRLSCTASGSIFSLATMTWYRQAPGKTREFVAAITWRGAATYAESVKGRFTISRDNAKNTMDLQMDGLKPEDTAVYYCNVKWGGGLNRAPFDYWGQGTQVTVSSGGSGGGGSGGGGSGGGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 226 - protein, 368 aa, synthetic construct
QVQLQESGGGLVQAGGSLRLSCAASISISKIDLMGWYRQAPGRQRELVARVTNGGDSYYSTSVKGRFTISRDNAKNTLYLQMNSLKPEDTAVYYCYGVPESLAAFHWGQGTLVTVSSGGSGGGGSGGGGSGGGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 227 - protein, 371 aa, synthetic construct
QVQLQESGGGLVQPGGSLRLSCAASGSIFSGNAMGWYRQAPGKQRELVASITSGGDTHYVDSVKGRFTISRDNAKNMVYLQMSSLKPEDTAVYYCNAQERVYSDYAFASWGPGTQVTVSSGGSGGGGSGGGGSGGGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 228 - protein, 366 aa, synthetic construct
QVQLQESGGGLVQPGGSLRLSCAASGFTFSTYVMNWVRQAPGKGLEWVSGIDSDGDSTTYTDSVKGRFTISRDNAKNTLYLQMNSLKPEDTAVYYCSMFRMATTGSQGTQVTVSSGGSGGGGSGGGGSGGGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 229 - protein, 370 aa, synthetic construct
QVQLQESGGGLVQAGGSLRLSCAASGITSSIYAMGWYRQAPGNEREPVALITSGDNTNYPDSVKGRFTISRDTAKNTVYLQMSSLKPEDTAVYYCYADVVIGTTYYTSWGQGTLVTVSSGGSGGGGSGGGGSGGGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 230 - protein, 215 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 231 - protein, 463 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGLNDIFEAQKIEWHE

SEQ ID NO: 232 - protein, 689 aa, synthetic construct
EVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 233 - protein, 450 aa, synthetic construct
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 234 - protein, 215 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 235 - protein, 360 aa, synthetic construct
QVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSSGGSGGGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 236 - protein, 358 aa, synthetic construct
QVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSSGGSGGGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 237 - protein, 350 aa, synthetic construct
QVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSSGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 238 - protein, 348 aa, synthetic construct
QVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSSGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 239 - protein, 370 aa, synthetic construct
QVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSSGGSGGGGSGGGGSGGGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 240 - protein, 368 aa, synthetic construct
QVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSSGGSGGGGSGGGGSGGGGSGGGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 241 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 242 - protein, 580 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGQVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSS

SEQ ID NO: 243 - protein, 582 aa, synthetic construct
QVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGQVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSS

SEQ ID NO: 244 - protein, 578 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGQVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSS

SEQ ID NO: 245 - protein, 580 aa, synthetic construct
QVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGQVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSS

SEQ ID NO: 246 - protein, 570 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGQVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSS

SEQ ID NO: 247 - protein, 572 aa, synthetic construct
QVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGQVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSS

SEQ ID NO: 248 - protein, 568 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGQVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSS

SEQ ID NO: 249 - protein, 570 aa, synthetic construct
QVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGQVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSS

SEQ ID NO: 250 - protein, 590 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSGGGGSGGQVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSS

SEQ ID NO: 251 - protein, 592 aa, synthetic construct
QVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSGGGGSGGQVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSS

SEQ ID NO: 252 - protein, 588 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSGGGGSGGQVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSS

SEQ ID NO: 253 - protein, 590 aa, synthetic construct
QVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSGGGGSGGQVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSS

SEQ ID NO: 254 - protein, 358 aa, synthetic construct
QVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSSGGSGGGGSGGGGSGGGGSGGGGSGGDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 255 - protein, 356 aa, synthetic construct
QVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSSGGSGGGGSGGGGSGGGGSGGGGSGGDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 256 - protein, 357 aa, synthetic construct
QVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSSGGSGGGGSGGGGSGGGGSGGGGSGGDIQMTQSPSSLSASVGDRVTITCRASQGINNYLNWYQQKPGKAPKRLIYNTNNLQTGVPSRFSGSGSGTEFTLTISSLQPEDFATYYCLQHNSFPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 257 - protein, 355 aa, synthetic construct
QVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSSGGSGGGGSGGGGSGGGGSGGGGSGGDIQMTQSPSSLSASVGDRVTITCRASQGINNYLNWYQQKPGKAPKRLIYNTNNLQTGVPSRFSGSGSGTEFTLTISSLQPEDFATYYCLQHNSFPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 258 - protein, 592 aa, synthetic construct
QVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSSGGSGGGGSGGGGSGGGGSGGGGSGGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 259 - protein, 590 aa, synthetic construct
QVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSSGGSGGGGSGGGGSGGGGSGGGGSGGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 260 - protein, 593 aa, synthetic construct
QVQLQQSGGGLVQAGGSLRLSCTASGSTFSFSNYHMGWYRQAPGKQRERVASISSRDSTYYSDSVKGRFTISRDTARNTVYLQMNSLEPEETAVYYCNARGRATGRDYWGQGTQVTVSSGGSGGGGSGGGGSGGGGSGGGGSGGQVQLVQSGAEVKKPGSSVKVSCKASGFTFTDYKIHWVRQAPGQGLEWMGYFNPNSGYSTYAQKFQGRVTITADKSTSTAYMELSSLRSEDTAVYYCARLSPGGYYVMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 261 - protein, 591 aa, synthetic construct
QVQLQEFGGGLVQAGEALRLSCVASKTIFNTMPMGWYRQAPGKERELVATITSSGVVNSADSVKGRFTISRDSAKRTAYLQMNNLKPEDTAVYYCAAMFKPGIPEYWGRGTQVTVSSGGSGGGGSGGGGSGGGGSGGGGSGGQVQLVQSGAEVKKPGSSVKVSCKASGFTFTDYKIHWVRQAPGQGLEWMGYFNPNSGYSTYAQKFQGRVTITADKSTSTAYMELSSLRSEDTAVYYCARLSPGGYYVMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 262 - protein, 5 aa, synthetic construct
GGGGS

SEQ ID NO: 263 - protein, 15 aa, synthetic construct
GGGGSGGGGSGGGGS

SEQ ID NO: 264 - protein, 25 aa, synthetic construct
GGGGSGGGGSGGGGSGGGGSGGGGS

SEQ ID NO: 265 - protein, 214 aa, synthetic construct
DIQMTQSPSSLSASVGDRVTITCRASQSISSYLNWYQQKPGKAPKLLIYTASSLQSGVPSRFSGSGSGTDFTLTISSLQPEDFATYYCQQSYSTPLTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 266 - protein, 231 aa, synthetic construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARDPGPPYNWYWGAYDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 267 - protein, 674 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQYNSDWWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGEVQLLESGGGLVQPGGSLRLSCAASGFTFSSYAMSWVRQAPGKGLEWVSAITGSGGSTYYADSVKGRFTISRDNSRNTLYLQMNSLRAEDTAVYYCAKGEGYAGSSYFRASDIWGQGTMVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 268 - protein, 224 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVYYYGFDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 269 - protein, 673 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQYASWYTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGEVQLLESGGGLVQPGGSLRLSCAASGFTFSSYAMSWVRQAPGKGLEWVSAITGSGGSTYYADSVKGRFTISRDNSRNTLYLQMNSLRAEDTAVYYCAKGEGYAGSSYFRASDIWGQGTMVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 270 - protein, 227 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 271 - protein, 213 aa, synthetic construct
QIVLTQSPAIMSASPGEKVTMTCSGSSSVSFMYWYQQRPGSSPRLLIYDTSNLASGVPVRFSGSGSGTSYSLTISRMEAEDAATYYCQQWSTYPLTFGAGTKLELKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 272 - protein, 673 aa, synthetic construct
QVQLKQSGPGLVQPSQSLSITCTVSGFSVTSYGVHWIRQSPGKGLEWLGVIWSGGSTDYNAAFISRLTISKDNSKSQVFFKVNSLQPADTAIYYCARAGDYNYDGFAYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCGGGGSGGGGDIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 273 - protein, 219 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQVLQPPPTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 274 - protein, 677 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDYSWWGDSYTGFDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCGGGGSGGGGDIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 275 - protein, 213 aa, synthetic construct
DIQMTQSPSSLSASVGDRVTITCRASQSISSYLNWYQQKPGKAPKLLIYTASSLQSGVPSRFSGSGSGTDFTLTISSLQPEDFATYYCQQSYSTPLTFGGGTKVEIKGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 276 - protein, 705 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARLLYYGFDYWGQGTLVTVSSGGSGGGGSGGGGSGGGGSGGDIQMTQSPSTLSASVGDRVTITCRASQSISSWLAWYQQKPGKAPKLLIYDASSLESGVPSRFSGSGSGTEFTLTISSLQPDDFATYYCQQYSSYYTFGQGTKVEIKGGGGSGGGGEVQLLESGGGLVQPGGSLRLSCAASGFTFSSYAMSWVRQAPGKGLEWVSAITGSGGSTYYADSVKGRFTISRDNSRNTLYLQMNSLRAEDTAVYYCAKGEGYAGSSYFRASDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 277 - protein, 449 aa, synthetic construct; identical to SEQ ID NO: 194

SEQ ID NO: 278 - protein, 481 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDYSWWGDSYTGFDYWGQGTLVTVSSGGSGGGGSGGGGSGGGGSGGDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQVLQPPPTFGQGTKVEIKGGGGSGGGGDIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 279 - protein, 25 aa, synthetic construct
GGSGGGGSGGGGSGGGGSGGGGSGG

SEQ ID NO: 280 - protein, 9 aa, synthetic construct
GGGGSGGGG

SEQ ID NO: 281 - protein, 20 aa, synthetic construct
GGSGGGGSGGGGSGGGGSGG

SEQ ID NO: 282 - protein, 124 aa, synthetic construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARDPGPPYNWYWGAYDYWGQGTTVTVSS

SEQ ID NO: 283 - protein, 107 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQYNSDWWTFGQGTKVEIK

SEQ ID NO: 284 - protein, 117 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVYYYGFDYWGQGTLVTVSS

SEQ ID NO: 285 - protein, 106 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQYASWYTFGQGTKVEIK

SEQ ID NO: 286 - protein, 119 aa, synthetic construct
QVQLKQSGPGLVQPSQSLSITCTVSGFSVTSYGVHWIRQSPGKGLEWLGVIWSGGSTDYNAAFISRLTISKDNSKSQVFFKVNSLQPADTAIYYCARAGDYNYDGFAYWGQGTLVTVSS

SEQ ID NO: 287 - protein, 106 aa, synthetic construct
QIVLTQSPAIMSASPGEKVTMTCSGSSSVSFMYWYQQRPGSSPRLLIYDTSNLASGVPVRFSGSGSGTSYSLTISRMEAEDAATYYCQQWSTYPLTFGAGTKLELK

SEQ ID NO: 288 - protein, 123 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDYSWWGDSYTGFDYWGQGTLVTVSS

SEQ ID NO: 289 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQVLQPPPTFGQGTKVEIK

SEQ ID NO: 290 - protein, 117 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARLLYYGFDYWGQGTLVTVSS

SEQ ID NO: 291 - protein, 106 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQYSSYYTFGQGTKVEIK

SEQ ID NO: 292 - protein, 112 aa, synthetic construct; identical to SEQ ID NO: 289

SEQ ID NO: 293 - protein, 123 aa, synthetic construct; identical to SEQ ID NO: 288